(19) United States
(12) Patent Application Publication
(10) Pub. No.: US 2025/0288683 A1
(43) Pub. Date: Sep. 18, 2025

(54) TETRAHYDROISOQUINOLINE HETEROBIFUNCTIONAL BCL-XL DEGRADERS

(71) Applicant: Treeline Biosciences, Inc., Watertown, MA (US)

(72) Inventors: Evan J. Horn, San Diego, CA (US); Joshua D. Hansen, San Diego, CA (US); Matthew D. Alexander, San Diego, CA (US); Fei Huang, San Diego, CA (US); Mark A. Nagy, San Diego, CA (US)

(21) Appl. No.: 18/858,971

(22) PCT Filed: May 4, 2023
(86) PCT No.: PCT/US2023/020956

§ 371 Date: Oct. 22, 2024

Related U.S. Application Data
(60) Provisional application No. 63/339,308, filed on May 6, 2022.
(60) Provisional application No. 63/398,752, filed on Aug. 17, 2022.
(60) Provisional application No. 63/429,834, filed on Dec. 2, 2022.
(60) Provisional application No. 63/449,728, filed on Mar. 3, 2023.
(60) Provisional application No. 63/454,486, filed on Mar. 24, 2023.

(51) Int. Cl. A61K 47/55; A61K 31/506; A61K 31/519; A61K 31/529; A61K 31/5377; A61K 47/54; A61P 35/00

(52) U.S. Cl. CPC A61K 47/55; A61K 31/506; A61K 31/519; A61K 31/529; A61K 31/5377; A61K 47/545; A61P 35/00

(57) ABSTRACT

This disclosure provides compounds of Formula (I) or pharmaceutically acceptable salts thereof, that induce degradation of a BCL-X_{L} protein. These are useful, for example, for treating a cancer in a subject (e.g., a human). This disclosure also compositions containing the compounds provided herein as well as methods of using and making the same.

### CROSS-REFERENCE TO RELATED APPLICATIONS

This application claims priority to U.S. Provisional Application Ser. No. 63/454,486, filed Mar. 24, 2023; 63/449,728, filed Mar. 3, 2023; 63/429,834, filed Dec. 2, 2022; 63/398,752, filed Aug. 17, 2022; and 63/339,308, filed May 6, 2022; each of which is incorporated by reference in its entirety herein.

### TECHNICAL FIELD

This disclosure provides compounds of Formula (I) or (II), or pharmaceutically acceptable salts thereof, that induce degradation of a BCL-X_{L} protein. These compounds are useful, for example, for treating cancer in a subject (e.g., a human). This disclosure also provides compositions containing the compounds provided herein as well as methods of using and making the same.

### BACKGROUND

The BCL-2 family of proteins is involved in the regulation of cell apoptosis and includes proteins that are pro-apoptosis, pro-survival, and BH3-only. At a high level, the balance of binding of BH3-only proteins to the pro-apoptosis and pro-survival members of the BCL-2 family can determine whether a cell will undergo apoptosis. The protein BCL-X_{L}, encoded by the BCL2L1 gene, is a pro-survival member of the BCL-2 family. In many cancers, it can be desirable to initiate apoptosis of tumor cells, which may be achieved by decreasing the amount of pro-survival protein (e.g., BCL-X_{L}) available to compete for BH3-only protein binding.

### SUMMARY

This disclosure provides compounds of Formula (I) or (II), or pharmaceutically acceptable salts thereof, that induce degradation of a BCL-X_{L} protein. These compounds are useful, e.g., for treating a cancer in a subject (e.g., a human). This disclosure also provides compositions containing the compounds provided herein as well as methods of using and making the same.

Provided herein are compounds of Formula (I) or (II):

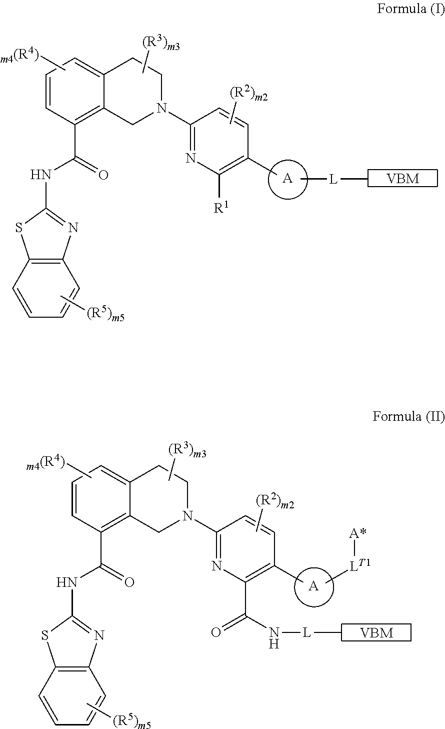

or a pharmaceutically acceptable salt thereof, wherein:
Ring A is selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) phenylene or 5-6 membered heteroarylene, each of which is optionally substituted with 1-3 substituents independently selected from the group consisting of: R^{a} and R^{b};
L^{T1} is a bond or C₁₋₃ alkylene optionally substituted with 1-3 substituents independently selected from the group consisting of: oxo and R^{c}, wherein one CH₂ unit of the C₁₋₃ alkylene is optionally replaced with —O— or —N(R^{d})—;
A* is selected from the group consisting of:
(a) C₃₋₁₅ cycloalkyl or 3-15 membered heterocyclyl, each of which is optionally substituted with 1-6 substituents independently selected from the group consisting of: R^{a} and R^{b};
(b) C₆₋₁₅ aryl or 5-15 membered heteroaryl, each of which is optionally substituted with 1-6 substituents independently selected from the group consisting of: R^{a} and R^{b}; and
(c) H;
R¹ is selected from the group consisting of:
(a) C(O)OH,
(b) C(O)NR^{d}R^{e}, and
(c) C(O)OC₁₋₆ alkyl, wherein the C₁₋₆ alkyl is optionally substituted with 1-3 R^{c};
each R², R³, R⁴, and R⁵ is independently selected from the group consisting of halo, CN, C₁₋₃ alkyl, C₁₋₃ haloalkyl, C₁₋₃ alkoxy, C₁₋₃ haloalkoxy, OH, and NR^{d}R^{e};
m2 is 0, 1, or 2;
m3 and m4 are independently 0, 1, 2, or 3;
m5 is 0, 1, 2, 3, or 4;
L is -(L^{A})ₙ₁-, wherein L^{A} and n1 are defined according to (AA) or (BB):
   (AA)
n1 is an integer from 3 to 12; and
each L^{A} is independently selected from the group consisting of: L^{A1}, LA, and L^{A4} provided that 1-3 occurrences of L^{A} is L^{A4};
   (BB)
n1 is an integer from 0 to 20; and
each L^{A} is independently selected from the group consisting of: L^{A1} and L^{A3};
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—S(O)₀₋₂—, and C(═O);
each L¹⁴ is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any O—O, N—O, N—N, N—S(O)₀, or O—S(O)₀₋₂ bonds;
wherein each R^{L} is independently selected from the group consisting of: halo; cyano; —OH; —C₁₋₆ alkoxy; —C₁₋₆ haloalkoxy; —NR^{d}R^{e}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{c};
VBM is selected from the group consisting of: (V1), (V2), (V3), (V4), (V5), (V6), (V7), (V8), and (V1P):

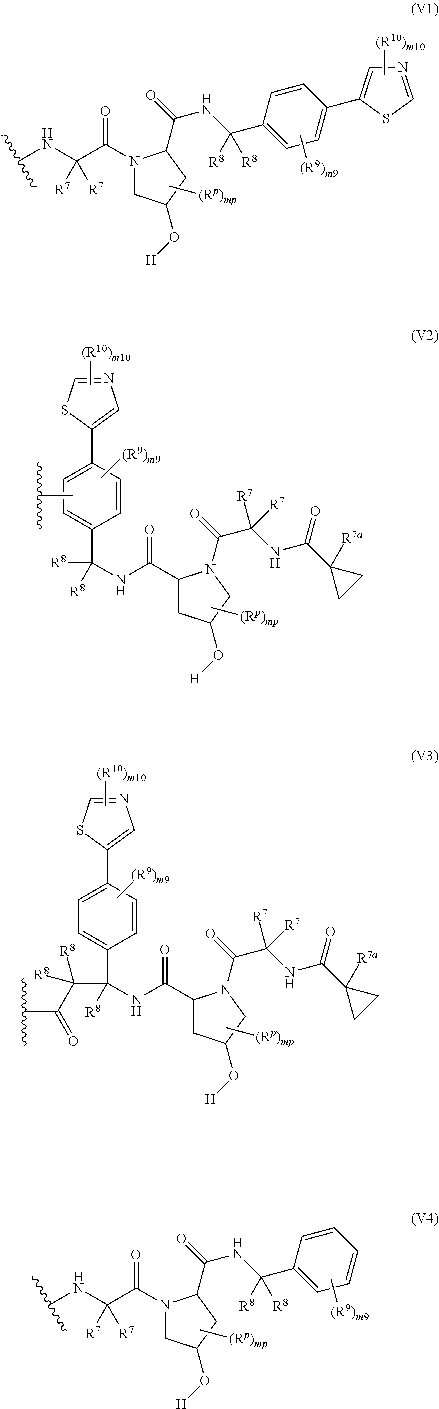

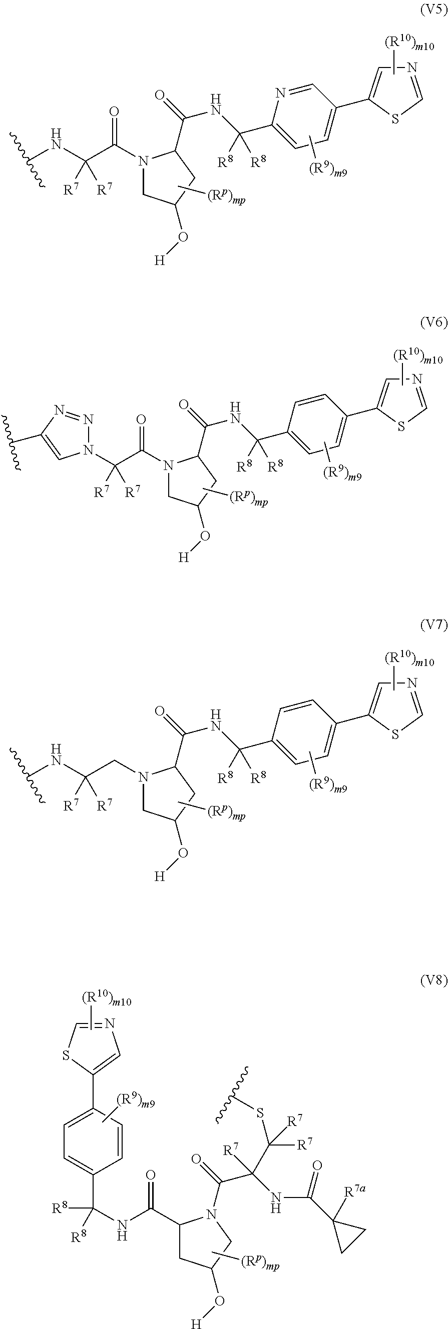

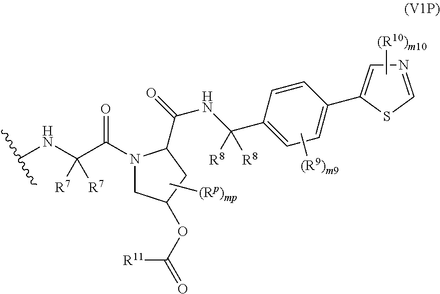

wherein:
each R⁷ and R⁸ is independently selected from the group consisting of: H, C₁₋₆ alkyl, C₁₋₆ haloalkyl, and C₃₋₆ cycloalkyl;
R^{7a} is selected from the group consisting of: halo and CN;
mp is 0, 1, or 2;
each R^{p} is independently selected from the group consisting of: —F, C₁₋₆ alkyl, C₁₋₆ haloalkyl, and C₃₋₆ cycloalkyl;
m9 is 0, 1, 2, or 3;
m10 is 0, 1, or 2; and
each R⁹ and R¹⁰ is independently selected from the group consisting of: halo, CN, C₁₋₆ alkyl, C₁₋₆ haloalkyl, C₃₋₆ cycloalkyl, C₁₋₆ alkoxy, and C₁₋₆ haloalkoxy;
R¹¹ is C₁₋₁₀ alkyl optionally substituted with 1-6 R^{c};
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)CL-6 alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1, 2, or 3;
each -Ln is independently selected from the group consisting of: —O—; —N(H)—; —N(C₁₋₃ alkyl)-; —S(O)₀₋₂—; C(═O); and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(R^{f})₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h},
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

Also provided herein is a pharmaceutical composition comprising a compound of Formula (I) or (II), or a pharmaceutically acceptable salt thereof, and a pharmaceutically acceptable carrier.

Provided herein is a method for treating cancer in a subject in need thereof, the method comprising administering to the subject a therapeutically effective amount of a compound of Formula (I) or (II), or a pharmaceutically acceptable salt thereof, or a pharmaceutical composition as provided herein.

Also provided herein is a BCL-X_{L} protein non-covalently bound with a compound of Formula (I) or (II), or a pharmaceutically acceptable salt thereof.

Also provided herein is a ternary complex comprising a BCL-X_{L} protein, a compound of Formula (I) or (II), or a pharmaceutically acceptable salt thereof, and a VHL protein, or a portion thereof.

To facilitate understanding of the disclosure set forth herein, a number of additional terms are provided. Generally, the nomenclature used herein and the laboratory procedures in organic chemistry, medicinal chemistry, and pharmacology described are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

The details of one or more embodiments of the invention are set forth in the accompanying drawings and the description below. Other features and advantages of the invention will be apparent from the description and drawings, and from the claims.

### DETAILED DESCRIPTION

This disclosure provides compounds of Formula (I) or (II), or pharmaceutically acceptable salts thereof, that induce degradation of a BCL-X_{L} (also referred to as Bcl-xL herein) protein. These compounds are useful, e.g., for treating a cancer. This disclosure also provides compositions containing the compounds provided herein as well as methods of using and making the same.

Without being bound by any particular theory, it is believed that in healthy cells, pro-apoptotic effectors such as BAX and BAK can move between the cytosol and the mitochondrial outer membrane (MOM), on which Voltage Dependent Anion Channel 2 (VDAC2) can act as a receptor. Pro-survival BCL-2 family members (e.g., BCL-2, BCL-X_{L}, and MCL-1) can retrotranslocate BAX back to the cytosol. A BH3-only protein (e.g., BIM) can engage a rear site on a pro-apoptotic effector (e.g., BAX or BAK) and release the C-terminal transmembrane domain (09) of the effector, enabling binding to the MOM. Binding of BIM to the canonical BH3 binding groove of BAX or BAK releases the N-terminus and al of BAX or BAK, and subsequent unfolding of the “latch” domain releases BIM from the BH3 binding groove. If a pro-survival BCL-2 family member then binds to BAX or BAK, apoptotic signaling is generally halted. However, if BAX or BAK are allowed to dimerize, then oligomerize, the MOM can be permeabilized, leading to apoptosis.

An abundance of pro-survival BCL-2 family members is sometimes thought to “prime” cells for death (e.g., via cytotoxic therapies, including BH3 mimetics). It is believed that oncogenic mutations and stresses can cause an upregulation in BH3-only proteins, thus placing a selective pressure on cancer cells for upregulation of the pro-survival BCL-2 family proteins. Thus, with more BH3-only proteins around, the cells are believed to be more sensitive to further manipulations of the BCL-2 family balance. See, e.g., Adams and Cory, Cell Death & Differentiation 25.1 (2018): 27-36.

Compounds that induce degradation of a target protein are sometimes referred to as heterobifunctional compounds, PROTACs, or degraders. Such compounds generally include a moiety that binds to the target protein and a moiety that binds to a ubiquitin E3 ligase (sometimes referred to as an E3 ligase or simply an E3), these two moieties being optionally separated by a linker. To induce degradation, heterobifunctional compounds are believed to induce formation of a ternary complex between the target protein, the compound, and an E3 ligase. Formation of the ternary complex is then followed by ubiquitination of the target protein and degradation of the ubiquitinated target protein by a proteosome. Several E3 ligases have been used as the partner E3 ligase for heterobifunctional degraders. Herein, the von Hippel-Lindau (VHL) E3 ligase (also referred to herein as a VHL protein) is used.

A degradation approach for a target protein can have potential advantages compared to, e.g., small molecule inhibition of the target protein. One potential advantage is that the duration of effect of a heterobifunctional compound is generally based on the resynthesis rate of the target protein. Another potential advantage is that many heterobifunctional compounds are believed to be released from the ubiquitinated target protein-E3 ligase complex and made available for formation of further ternary complexes; this is sometimes referred to as “catalytic” turnover of the heterobifunctional compound. Degradation of a target protein can also be advantageous over small molecule inhibition in some cases, as degradation can impair a scaffolding function of a target protein, whereas a small molecule might not. It is also generally believed that for formation of a ternary complex, high affinity to the target protein is not always required.

Heterobifunctional compounds are further described in, for example, International Publication Nos. WO 2017/184995; WO 2019/144117; WO 2020/163823; WO 2021/078301; WO 2021/146536; WO 2021/007307; WO 2021/222114; WO 2021/078301; WO 2022/169780; WO 2023/044046; Chamberlain and Hamann, Nature Chemical Biology 15.10 (2019): 937-944; Li and Song, Journal of Hematology & Oncology 13 (2020): 1-14; Wu, et al. Nature Structural & Molecular Biology 27.7 (2020): 605-614; Dong, et al., Journal of Medicinal Chemistry 64.15 (2021): 10606-10620; Yang, et al., Targeted Oncology 16.1 (2021): 1-12; Lv, et al., Nature Communications 12.1 (2021): 6896.

### Compound Embodiments

Provided herein are compounds of Formula (I) or (II):

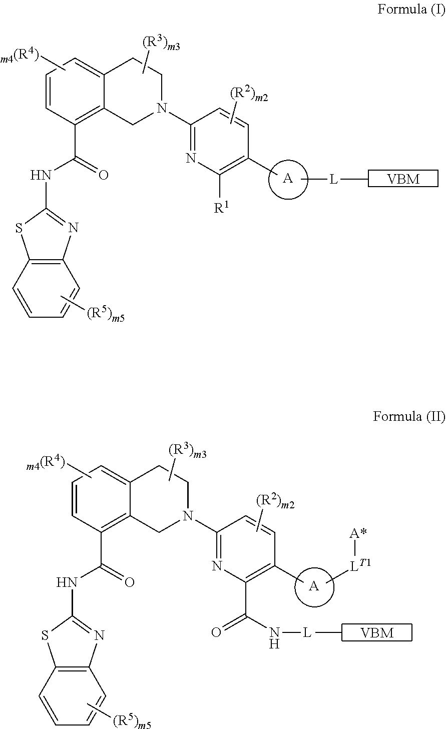

or a pharmaceutically acceptable salt thereof, wherein:
   Ring A is selected from the group consisting of:
   (a) C₃₋₁₅ cycloalkylene or 3-15 membered heterocyclylene, each of which is optionally substituted with 1-6 substituents independently selected from the group consisting of: R^{a} and R^{b}; and
   (b) phenylene or 5-6 membered heteroarylene, each of which is optionally substituted with 1-3 substituents independently selected from the group consisting of: R^{a} and R^{b};
   L^{T1} is a bond or C₁₋₃ alkylene optionally substituted with 1-3 substituents independently selected from the group consisting of: oxo and R^{c}, wherein one CH₂ unit of the C₁₋₃ alkylene is optionally replaced with —O— or —N(R^{d})—;
   A* is selected from the group consisting of:
   (a) C₃₋₁₅ cycloalkyl or 3-15 membered heterocyclyl, each of which is optionally substituted with 1-6 substituents independently selected from the group consisting of: R^{a} and R^{b};
   (b) C₆₋₁₅ aryl or 5-15 membered heteroaryl, each of which is optionally substituted with 1-6 substituents independently selected from the group consisting of: R^{a} and R^{b}; and
   (c) H;
   R¹ is selected from the group consisting of:
   (a) C(O)OH,
   (b) C(O)NR^{d}R^{e}, and
   (c) C(O)OC₁₋₆ alkyl, wherein the C₁₋₆ alkyl is optionally substituted with 1-3 R^{e};
   each R², R³, R⁴, and R⁵ is independently selected from the group consisting of halo, CN, C₁₋₃ alkyl, C₁₋₃ haloalkyl, C₁₋₃ alkoxy, C₁₋₃ haloalkoxy, OH, and NR^{d}R^{e};
   m2 is 0, 1, or 2;
   m3 and m4 are independently 0, 1, 2, or 3;
   m5 is 0, 1, 2, 3, or 4;
   L is -(L^{A})ₙ₁-, wherein L^{A} and n1 are defined according to (AA) or (BB):
      (AA)
   n1 is an integer from 3 to 12; and
   each L^{A} is independently selected from the group consisting of: L^{A1}, LA, and L^{A4} provided that 1-3 occurrences of L^{A} is L^{A4};
      (BB)
   n1 is an integer from 0 to 20; and
   each L^{A} is independently selected from the group consisting of: L^{A1} and L^{A3};
   each L^{A1} is independently selected from the group consisting of: —CH₂—, —CHR^{L}—, and —C(R^{L})₂—;
   each L^{A3} is independently selected from the group consisting of: —N(R^{d})—, —N(R^{b})—, —O—, —S(O)₀₋₂—, and C(═O);
   each L^{A4} is independently selected from the group consisting of:
   (a) C₃-15 cycloalkylene or 3-15 membered heterocyclylene, each of which is optionally substituted with 1-6 substituents independently selected from the group consisting of: R and R^{b}; and
   (b) C₆₋₁₅ arylene or 5-15 membered heteroarylene, each of which is optionally substituted with 1-6 substituents independently selected from the group consisting of: R^{a} and R^{b};
   provided that L does not contain any O—O, N—O, N—N, N—S(O)₀, or O—S(O)₀₋₂ bonds;
   wherein each R^{L} is independently selected from the group consisting of: halo; cyano; —OH; —C₁₋₆ alkoxy; —C₁₋₆ haloalkoxy; —NR^{d}R^{e}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{c};
   VBM is selected from the group consisting of: (V1), (V2), (V3), (V4), (V5), (V6), (V7), (V8), and (V1P):

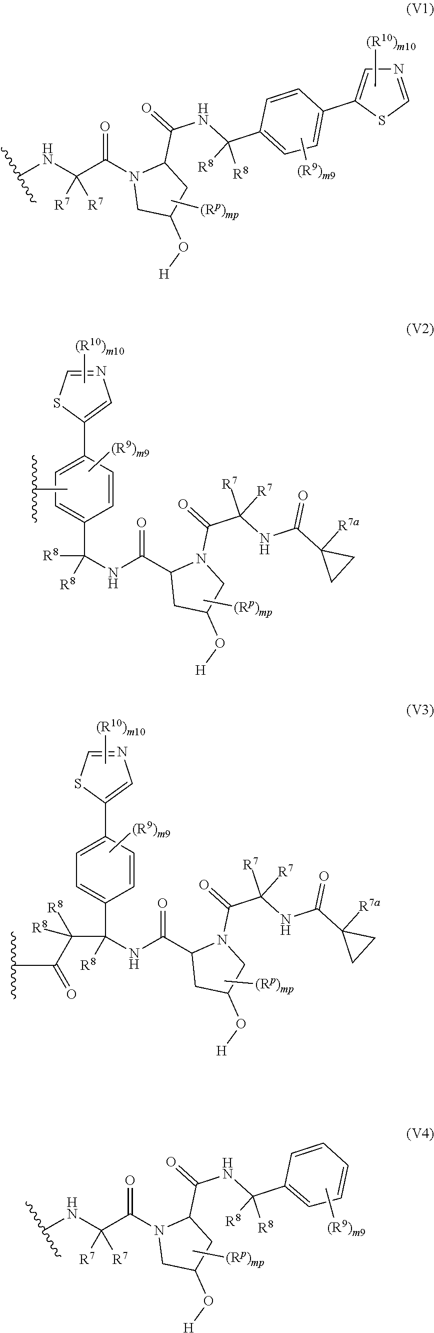

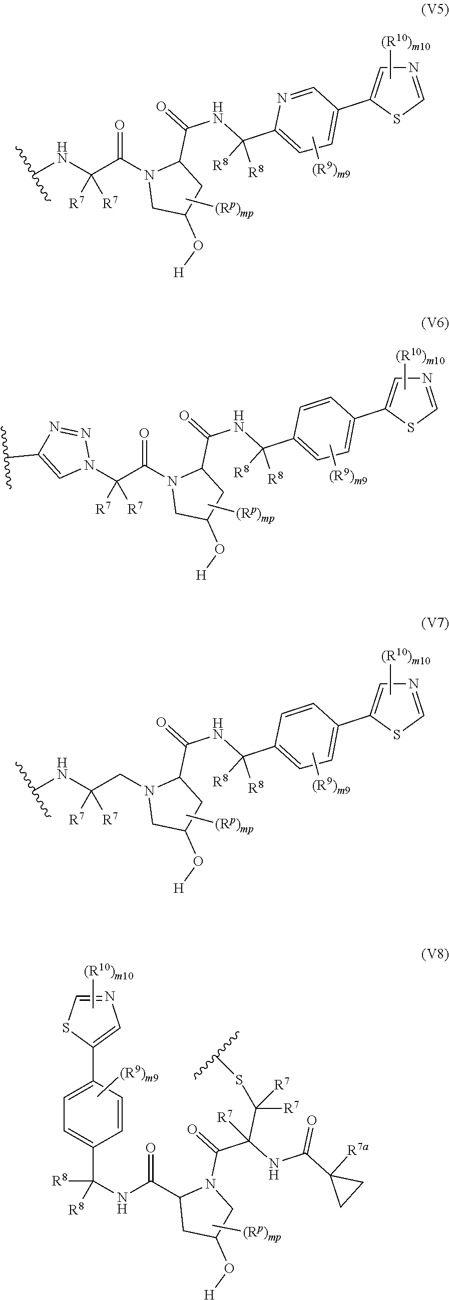

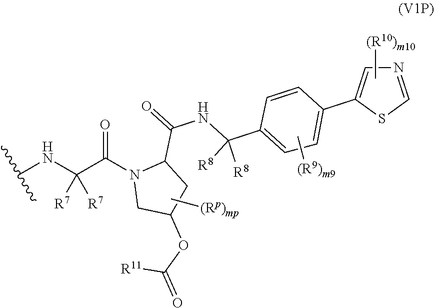

each R⁷ and R⁸ is independently selected from the group consisting of: H, C₁₋₆ alkyl, C₁₋₆ haloalkyl, and C₃₋₆ cycloalkyl;
R^{7a} is selected from the group consisting of: halo and CN;
mp is 0, 1, or 2;
each R^{p} is independently selected from the group consisting of: —F, C₁₋₆ alkyl, C₁₋₆ haloalkyl, and C₃₋₆ cycloalkyl;
m9 is 0, 1, 2, or 3;
m10 is 0, 1, or 2; and
each R⁹ and R¹⁰ is independently selected from the group consisting of: halo, CN, C₁₋₆ alkyl, C₁₋₆ haloalkyl, C₃₋₆ cycloalkyl, C₁₋₆ alkoxy, and C₁₋₆ haloalkoxy;
R¹¹ is C₁₋₁₀ alkyl optionally substituted with 1-6 R^{e};
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{e};
each R^{b} is independently selected from the group consisting of: -(L_{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(R^{f})₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h},
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁-3 haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

In some embodiments, VBM is selected from the group consisting of: (V1), (V2), (V3), and (V4).

In some embodiments, it is provided that when VBM is (V1), (V2), or (V3):
(i) in Formula (I), L does not contain an adamantylene group; and
(ii) in Formula (II), A* is other than adamantyl.

In some embodiments, it is provided that:
(i) in Formula (I), L does not contain an adamantylene group; and
(ii) in Formula (II), A* is other than adamantyl.

In some embodiments, the compounds are compounds of Formula (I) or pharmaceutically acceptable salts thereof.

In some embodiments, the compounds are compounds of Formula (II) or pharmaceutically acceptable salts thereof.

In some embodiments, Ring A is a phenylene optionally substituted with 1-3 R^{a}.

In some embodiments, Ring A is

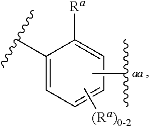

wherein aa represents the point of attachment to L or L^{T1}.

In some embodiments, Ring A is

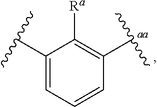

wherein aa represents the point of attachment to L or L^{T1}. For example, Ring A can be

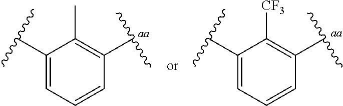

or wherein aa represents the point of attachment to L or L^{T1}.

In some embodiments, Ring A is

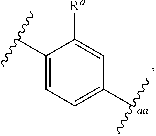

wherein aa represents the point of attachment to L or L^{T1}. For example, Ring A can be

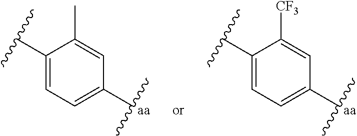

wherein aa represents the point of attachment to L or L^{T1}.

In some embodiments, Ring A is 5-6 membered heteroarylene optionally substituted with 1-3 R^{a}. In some embodiments, Ring A is 5-6 membered heteroarylene optionally substituted with 1-2 R^{a}. In some embodiments, Ring A is 5-membered heteroarylene optionally substituted with 1-2 R^{a}. In some embodiments, Ring A is pyrazolylene optionally substituted with 1-2 R^{a}. For example, Ring A is selected from the group consisting of:

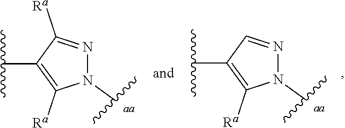

wherein aa represents the point of attachment to L or L^{T1}.

In some embodiments, Ring A is C₃₋₁₀ cycloalkylene optionally substituted with 1-6 R^{a}. In some embodiments, Ring A is C₄₋₆ cycloalkylene optionally substituted with 1-3 R^{a}. In some embodiments, Ring A is cyclohexylene optionally substituted with 1-3 R^{a}. For example, Ring A can be 1,4-cyclohexylene.

In some embodiments, one R^{a} present on Ring A is C₁₋₃ alkyl optionally substituted with 1-3 F. In some embodiments, one R^{a} present on Ring A is methyl or CF₃.

In some embodiments, R¹ is C(O)OH. In some embodiments, R¹ is C(O)NHR^{e}.

In some embodiments, R¹ is C(O)OH; and Ring A is

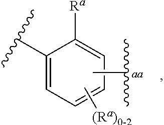

wherein aa represents the point of attachment to L or L^{T1}. In some embodiments, Ring A is

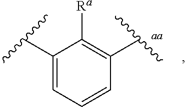

wherein aa represents the point of attachment to L or L^{T1}. For example, Ring A can be

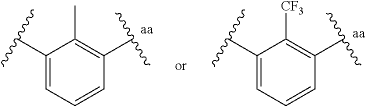

wherein aa represents the point of attachment to L or L^{T1}. In some embodiments, Ring A is

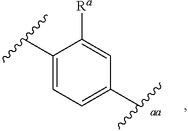

wherein aa represents the point of attachment to L or L^{T1}. For example, Ring A can be

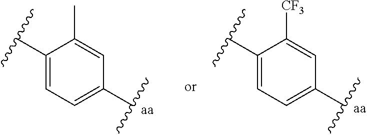

wherein aa represents the point of attachment to L or L^{T1}.

In some embodiments, L^{T1} is C₁₋₃ alkylene. For example, L^{T1} can be —CH₂—.

In some embodiments, A* is H. In some embodiments, A* is C₃₋₁₅ cycloalkyl or 3-15 membered heterocyclyl, each of which is optionally substituted with 1-6 substituents independently selected from the group consisting of: R^{a} and R^{b}. In some embodiments, A* is C₆₋₁₅ aryl or 5-15 membered heteroaryl, each of which is optionally substituted with 1-6 substituents independently selected from the group consisting of: R^{a} and R^{b}. For example, A* can be phenyl optionally substituted with 1-3 R^{a}.

In some embodiments, m2 is 0. In some embodiments, m3 is 0. In some embodiments, m4 is 0. In some embodiments, m5 is 0.

In some embodiments, m2 is 0; m3 is 0; m4 is 0; and m5 is 0.

In some embodiments, VBM is (V1):

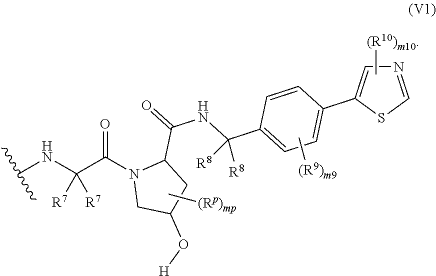

In some embodiments, VBM is (V1a):

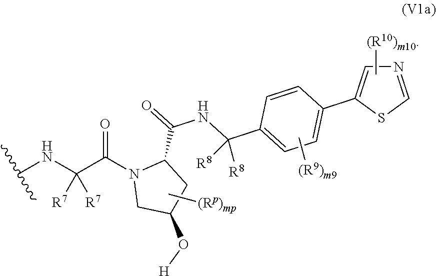

In some embodiments, VBM is (V2):

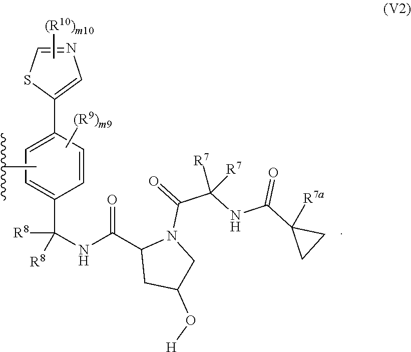

In some embodiments, VBM is (V2a):

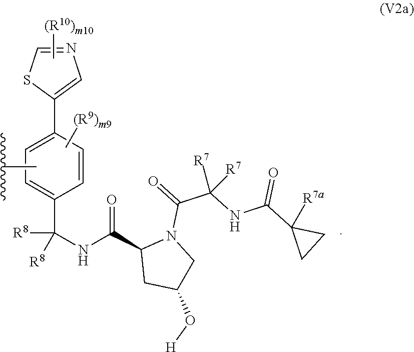

In some embodiments, VBM is (V3):

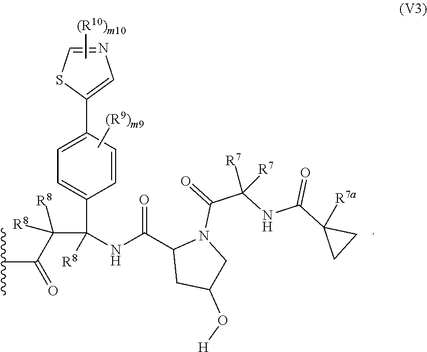

In some embodiments, VBM is (V3a):

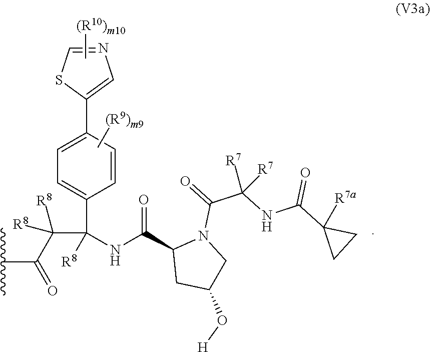

In some embodiments, VBM is (V4):

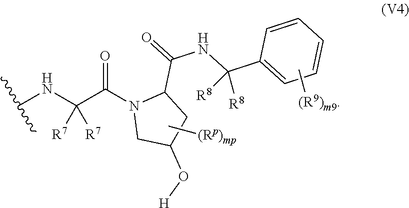

In some embodiments, VBM is (V4a):

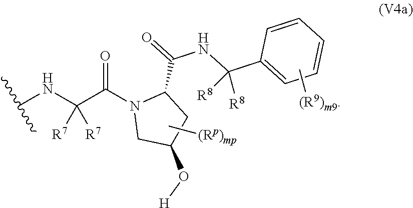

In some embodiments, one R⁷ is H; and the other R⁷ is C₁₋₆ alkyl. In some embodiments, one R⁷ is H; and the other R⁷ is tert-butyl.

In some embodiments, the carbon atom to which each R⁷ is attached has (S)-stereogenic configuration.

In some embodiments, one R⁸ is H; and the other R⁸ is C₁₋₆ alkyl. In some embodiments, one R⁸ is H; and the other R⁸ is methyl. In some embodiments, each R⁸ is H.

In some embodiments, the carbon atom to which each R⁸ is attached has (S)-stereogenic configuration.

In some embodiments, one R⁷ is H; the other R⁷ is C₁₋₆ alkyl; the carbon atom to which each R⁷ is attached has (S)-stereogenic configuration; one R⁸ is H; the other R⁸ is C₁₋₆ alkyl; and the carbon atom to which each R⁸ is attached has (S)-stereogenic configuration.

In some embodiments, m9 is 0.

In some embodiments, m10 is 1. In some embodiments, R¹⁰ is C₁₋₆ alkyl. For example, R¹⁰ can be methyl.

In some embodiments, mp is 0.

In some embodiments, m9 is 0; m10 is 1; and mp is 0.

In some embodiments, VBM is:

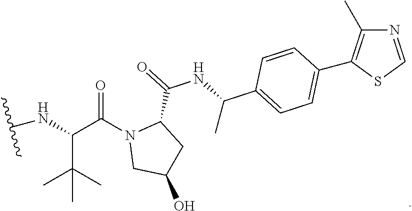 [SMILES: *N[C@H](C(=O)N1C[C@H](O)C[C@H]1C(=O)N[C@@H](C)c1ccc(-c2scnc2C)cc1)C(C)(C)C]

In some embodiments, VBM is:

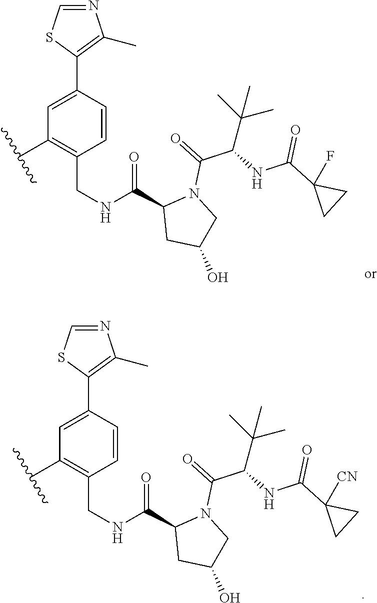 [SMILES: *c1cc(-c2scnc2C)ccc1CNC(=O)[C@@H]1C[C@@H](O)CN1C(=O)[C@@H](NC(=O)C1(F)CC1)C(C)(C)C ; *c1cc(-c2scnc2C)ccc1CNC(=O)[C@@H]1C[C@@H](O)CN1C(=O)[C@@H](NC(=O)C1(C#N)CC1)C(C)(C)C]

In some embodiments, VBM is:

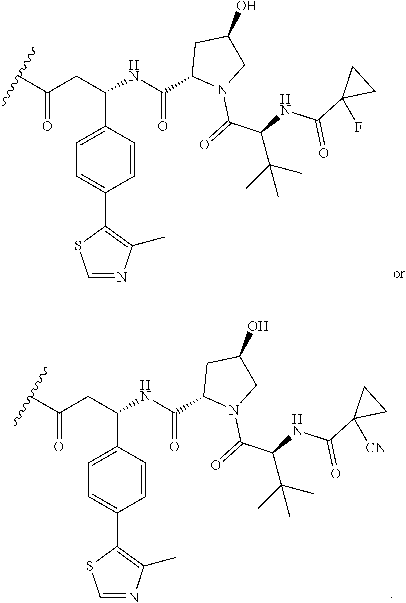 [SMILES: *C(=O)C[C@H](NC(=O)[C@@H]1C[C@@H](O)CN1C(=O)[C@@H](NC(=O)C1(F)CC1)C(C)(C)C)c1ccc(-c2scnc2C)cc1 ; *C(=O)C[C@H](NC(=O)[C@@H]1C[C@@H](O)CN1C(=O)[C@@H](NC(=O)C1(C#N)CC1)C(C)(C)C)c1ccc(-c2scnc2C)cc1]

In some embodiments, VBM is:

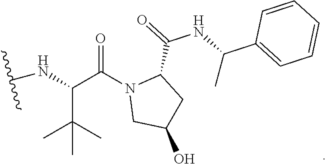 [SMILES: *N[C@H](C(=O)N1C[C@H](O)C[C@H]1C(=O)N[C@@H](C)c1ccccc1)C(C)(C)C]

In some embodiments, L is -(L^{A})ₙ₁-; and L^{A} and n are defined according to (BB).

In some embodiments, L^{A} and n1 are defined according to (BB); and n1 is an integer from 3 to 5. In some embodiments, L^{A} and n1 are defined according to (BB); and n1 is an integer from 5 to 9. In some embodiments, L^{A} and n1 are defined according to (BB); and n1 is an integer from 9 to 15.

In some embodiments, L^{A} and n1 are defined according to (BB); and 1-4 occurrences of L^{A} is L^{A3}. In some embodiments, 1-3 occurrences of L^{A} is L^{A3}. In some embodiments, 1-2 occurrences of L^{A} is L^{A3}.

In some embodiments, L^{A} and n1 are defined according to (BB); and 0-2 occurrences of L^{A1} is —CHR^{L}— or —C(R^{L})₂—; and each remaining occurrence of L^{A1} is —CH₂—. In some embodiments, each occurrence of L^{A1} is —CH₂—. In some embodiments, one occurrence of L^{A1} is —CHR^{L}— or —C(R^{L})₂—; and each remaining occurrence of L^{A1} is —CH₂—. In some embodiments, each R^{L} is independently selected from the group consisting of: —F and —C₁₋₃ alkyl optionally substituted with 1-3 F.

In some embodiments, L is -(L^{A3})₀₋₂-(L^{A1})₁₋₁₅-(L^{A3})_{0-1-bb}, wherein bb represents the point of attachment to VBM. In some embodiments, 0-2 occurrences of L^{A1} is —CHR^{L}— or —C(R^{L})₂—; and each remaining occurrence of L^{A1} is —CH₂—. In some embodiments, each occurrence of L^{A1} is —CH₂—. In some embodiments, one occurrence of L^{A1} is —CHR^{L}— or —C(R^{L})₂—; and each remaining occurrence of L^{A1} is —CH₂—. In some embodiments, each R^{L} is independently selected from the group consisting of: —F and —C₁₋₃ alkyl optionally substituted with 1-3 F.

In some embodiments, L is a divalent group of Formula (L-1):

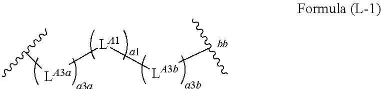

wherein:
a3a is 0 or 1;
L^{A3a} is selected from the group consisting of: —O—, —N(H)—, and —N(C₁₋₃ alkyl)-;
a1 is an integer from 1 to 15;
a3b is 0 or 1;
L^{A3b} is independently selected from the group consisting of: C(═O), —O—, N(H)—, and —N(C₁₋₃ alkyl)-; and
bb represents the point of attachment to VBM.

In some embodiments of (L-1), a3b is 1. In some embodiments, L^{A3b} is C(═O). In some embodiments, L^{A3b} is —N(H)—. In some embodiments, L^{A3b} is —N(C₁₋₃ alkyl)-. In some embodiments, L^{A3b} is —O—.

In some embodiments of (L-1), a3b is 0.

In some embodiments of (L-1), a3a is 1. In some embodiments, L^{A3a} is 0.

In some embodiments of (L-1), a3a is 0.

In some embodiments of (L-1), at is 1 or 2. In some embodiments of (L-1), at is an integer from 3 to 5. In some embodiments of (L-1), at is an integer from 5 to 9. In some embodiments of (L-1), at is an integer from 9 to 15.

In some embodiments of (L-1), 0-2 occurrences of L^{A1} is —CHR^{L}— or —C(R^{L})₂—; and each remaining occurrence of L^{A1} is —CH₂—. In some embodiments, each occurrence of L^{A1} is —CH₂—. In some embodiments, one occurrence of L^{A1} is —CHR^{L}— or —C(R^{L})₂—; and each remaining occurrence of L^{A1} is —CH₂—.

In some embodiments, each R^{L} is independently selected from the group consisting of: —F and —C₁₋₃ alkyl optionally substituted with 1-3 F.

In some embodiments, L is -(L^{A})ₙ₁-; and L^{A} and n1 are defined according to (AA). In some embodiments, L^{A} and n1 are defined according to (AA); and n1 is an integer from 3 to 5. In some embodiments, L^{A} and n1 are defined according to (AA); and n1 is an integer from 5 to 9. In some embodiments, L^{A} and n1 are defined according to (AA); and n1 is 6, 7, or 8. In some embodiments, L^{A} and n1 are defined according to (AA); and n1 is an integer from 9 to 12. In some embodiments, L^{A} and n1 are defined according to (AA); and 1-2 occurrences of L^{A} is L^{A4}. In some embodiments, one occurrence of L^{A} is L^{A4}.

In some embodiments, L^{A} and n1 are defined according to (AA); and each L^{A4} is independently selected from the group consisting of: C₃₋₁₀ cycloalkylene or 4-10 membered heterocyclylene, each of which is optionally substituted with 1-3 R^{a}; and phenylene or 5-6 membered heteroarylene, each of which is optionally substituted with 1-3 R^{a}.

In some embodiments, L^{A} and n1 are defined according to (AA); and 1-4 occurrences of L^{A} is L^{A3}. In some embodiments, 1-2 occurrences of L^{A} is L^{A3}.

In some embodiments, L^{A} and n1 are defined according to (AA); and 2-7 occurrences of L^{A} are L^{A1}. In some embodiments, 2-5 occurrences of L^{A} are L^{A1}. In some embodiments, 0-2 occurrences of L^{A1} is —CHR^{L}— or —C(R^{L})₂—; and each remaining occurrence of L^{A1} is —CH₂—.

In some embodiments, each occurrence of L^{A1} is —CH₂—. In some embodiments, one occurrence of L^{A1} is —CHR^{L}— or —C(R^{L})₂—; and each remaining occurrence of L^{A1} is —CH₂—. In some embodiments, each R^{L} is independently selected from the group consisting of: —F and —C₁₋₃ alkyl optionally substituted with 1-3 F.

In some embodiments, L is -(L^{A})ₙ₁-, and L^{A} and n1 are defined according to (AA), wherein: n1 is an integer from 5 to 9; 1-2 occurrences of L^{A} is L^{A4}; 2-7 occurrences of L^{A} are L^{A1}; and 1-3 occurrences of L^{A} is L^{A3}. In some embodiments, each L^{A4} is independently selected from the group consisting of: C₃₋₁₀ cycloalkylene or 4-10 membered heterocyclylene, each of which is optionally substituted with 1-3 R^{a}; and phenylene or 5-6 membered heteroarylene, each of which is optionally substituted with 1-3 R^{a}. In some embodiments, 0-1 occurrence of L^{A1} is —CHR^{L}— or —C(R^{L})₂—; and each remaining occurrence of L^{A1} is —CH₂—. In some embodiments, 0-1 occurrence of L^{A3} is C(═O); and each remaining occurrence of L^{A3} is independently selected from the group consisting of: —O—; —N(H)—; and —N(C₁₋₃ alkyl)-.

In some embodiments, L is selected from the group consisting of:
(i) -(L^{A3})₀₋₂-(L^{A1})₀₋₅-L^{A4}-(L^{A1})₀₋₅-L^{A3}-bb;
(ii) -(L^{A3})-2-(L^{A1})₀₋₅L^{A4}-(L^{A1})_{0-5-bb}; and
provided that L contains 2-7 L^{A1}; and wherein bb represents the point of attachment to VBM. In some embodiments, L^{A4} is selected from the group consisting of: C₃₋₁₀ cycloalkylene or 4-10 membered heterocyclylene, each of which is optionally substituted with 1-3 R^{a}; and phenylene or 5-6 membered heteroarylene, each of which is optionally substituted with 1-3 R^{a}. In some embodiments, 0-1 occurrence of L^{A1} is —CHR^{L}— or —C(R^{L})₂—; and each remaining occurrence of L^{A1} is —CH₂—. In some embodiments, 0-1 occurrence of L^{A3} is C(═O); and each remaining occurrence of L^{A3} is independently selected from the group consisting of: —O—; —N(H)—; and —N(C₁₋₃ alkyl)-.

In some embodiments, L is -(L^{A3})₀₋₂-(L^{A1})₀₋₅-L^{A4}-(L^{A1})₀₋₅-L^{A3}-_{bb}; provided that L contains 2-7 L^{A1}; and wherein bb represents the point of attachment to VBM. In some embodiments, L is -(L^{A3})₀₋₁-(L^{A1})₀₋₅-L^{A4}-(L^{A1})₀₋₅-L^{A3}-_{bb}; provided that L contains 2-5 L^{A1}; and wherein bb represents the point of attachment to VBM. In some embodiments, each L^{A4} is independently selected from the group consisting of: C₃₋₁₀ cycloalkylene or 4-10 membered heterocyclylene, each of which is optionally substituted with 1-3 R^{a}; and phenylene or 5-6 membered heteroarylene, each of which is optionally substituted with 1-3 R^{a}. In some embodiments, 0-1 occurrence of L^{A1} is —CHR^{L}— or —C(R^{L})₂—; and each remaining occurrence of L^{A1} is —CH₂—. In some embodiments, 0-1 occurrence of L^{A3} is C(═O); and each remaining occurrence of L^{A3} is independently selected from the group consisting of: —O—; —N(H)—; and —N(C₁₋₃ alkyl)-.

In some embodiments, L is a divalent group of Formula (L-2):

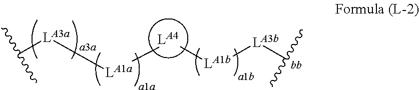

wherein:
a3a is 0 or 1;
L^{A3a} and L^{A3b} are independently selected from the group consisting of: —O—, —N(H)—, —N(C₁₋₃ alkyl)-, and C(═O);
a1a and a1b are independently integers from 0 to 5, provided that a1a+a1b is from 2 to 5;
L^{A1a} and L^{A1b} are independently selected from the group consisting of: —CH₂—, —CHR^{L}, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
bb represents the point of attachment to VBM.

In some embodiments of (L-2), a3a is 1. In some embodiments, L^{A3a} is —O—.

In some embodiments of (L-2), a3a is 0.

In some embodiments of (L-2), L^{A3b} is —N(H)— or —N(C₁₋₃ alkyl)-.

In some embodiments of (L-2), L^{A3b} is —O—.

In some embodiments of (L-2), L^{A3b} is C(═O).

In some embodiments of (L-2), a3a is 1; L^{A3a} is —O—; and L^{A3b} is C(═O).

In some embodiments of (L-2), a3a is 1; L^{A3a} is —O—; and L^{A3b} is —O—, —N(H)—, or —N(C₁₋₃ alkyl)-.

In some embodiments of (L-2), a3a is 0; and L^{A3b} is C(═O).

In some embodiments of (L-2), a3a is 0; and L^{A3b} is —O—, —N(H)—, or —N(C₁₋₃ alkyl)-.

In some embodiments, L is -(L^{A3})₀₋₂-(L^{A1})₀₋₅-L^{A4}-(L^{A1})_{0-5-bb}; provided that L contains 2-7 L^{A1}; and wherein bb represents the point of attachment to VBM. In some embodiments, L is -(L^{A3})₀₋₁-(L^{A1})₀₋₅-L^{A4}-(L^{A1})_{0-5-bb}; provided that L contains 2-5 L^{A1}; and wherein bb represents the point of attachment to VBM. In some embodiments, each L^{A4} is independently selected from the group consisting of: C₃₋₁₀ cycloalkylene or 4-10 membered heterocyclylene, each of which is optionally substituted with 1-3 R^{a}; and phenylene or 5-6 membered heteroarylene, each of which is optionally substituted with 1-3 R^{a}. In some embodiments, 0-1 occurrence of L^{A1} is —CHR^{L}— or —C(R^{L})₂—; and each remaining occurrence of L^{A1} is —CH₂—.

In some embodiments, L is a divalent group of Formula (L-3):

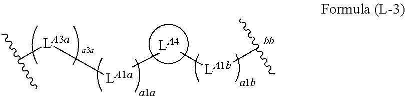

wherein:
a3a is 0 or 1;
L^{A3a} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
bb represents the point of attachment to VBM.

In some embodiments of (L-3), a3a is 1. In some embodiments, L^{A3a} is —O—.

In some embodiments of (L-3), a3a is 0.

In some embodiments of (L-2) or (L-3), a1a+a1b is 2, 3, or 4. In some embodiments of (L-2) or (L-3), a1a+a1b is 5.

In some embodiments of (L-2) or (L-3), each occurrence of L^{A1a} and L^{A1b} is —CH₂—.

In some embodiments of (L-2) or (L-3), one occurrence of L^{A1a} is —CHR^{L}— or —C(R^{L})₂—; each remaining occurrence of L^{A1a} is —CH₂—; and each occurrence of L^{A1b} is —CH₂—.

In some embodiments of (L-2) or (L-3), one occurrence of L^{A1b} is —CHR^{L}— or —C(R^{L})₂; each remaining occurrence of L^{A1b} is —CH₂—; and each occurrence of L^{A1a} is —CH₂—.

In some embodiments of (L-2) or (L-3), each R^{L} is independently selected from the group consisting of: —F and —C₁₋₃ alkyl optionally substituted with 1-3 F.

In some embodiments of (L-2) or (L-3), a1b is 2, 3, or 4; one occurrence of L^{A1b} is —CHR^{L}— or —C(R^{L})₂—; each remaining occurrence of L^{A1b} is —CH₂—; and each occurrence of L^{A1a} is —CH₂—. For example, one occurrence of L^{A1b} can be CF₂, CMe₂, C(H)F, C(H)Me, or C(H)CF₃.

In some embodiments of (L-2) or (L-3), L^{A4} is 4-10 membered heterocyclylene optionally substituted with 1-3 R^{a}. In some embodiments, L^{A4} is selected from the group consisting of:

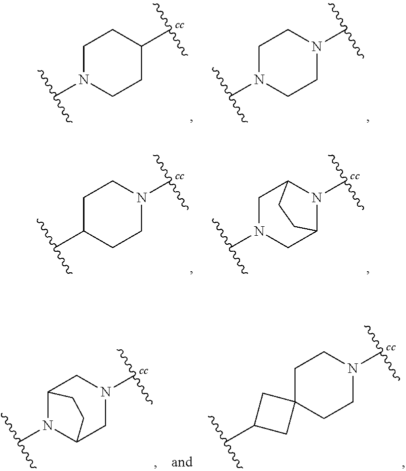 [SMILES: *C1CCN(*)CC1 ; *N1CCN(*)CC1 ; *C1CCN(*)CC1 ; *N1CC2CCC(C1)N2* ; *N1CC2CCC(C1)N2* ; *C1CC2(CCN(*)CC2)C1]

each optionally substituted with 1-3 R^{a} at one or more ring carbon atoms, wherein cc represents the point of attachment to L^{A1b}. In some embodiments, each R^{a} present on L^{A4} is independently F or C₁₋₃ alkyl optionally substituted with 1-3 F.

In some embodiments of (L-2) or (L-3), L^{A4} is C₃-10 cycloalkylene optionally substituted with 1-3 R^{a}. For example, L^{A4} can be 1,4-cyclohexylene optionally substituted with 1-3 R^{a}. For example, L^{A4} can be

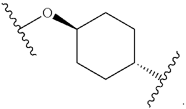 [SMILES: *O[C@H]1CC[C@H](*)CC1]

In some embodiments of (L-2) or (L-3), L^{A4} is phenylene or 5-6 membered heteroarylene, each of which is optionally substituted with 1-3 R^{a}. In some embodiments, L^{A4} is phenylene optionally substituted with 1-3 R^{a}. For example, L^{A4} can be 1,2-phenylene optionally substituted with 1-3 R^{a}. For example, L^{A4} can be 1,3-phenylene optionally substituted with 1-3 R^{a}. For example, L^{A4} can be 1,4-phenylene optionally substituted with 1-3 R^{a}.

In some embodiments, VBM is (V1); and L is (L-1), wherein a3b is 1; and L^{A3b} is C(═O). In some embodiments, VBM is (V1a); and L is (L-1), wherein a3b is 1; L^{A3b} is C(═O); a3a is 1; L^{A3a} is —O—; and at is 4, 5, or 6. In some embodiments, VBM is

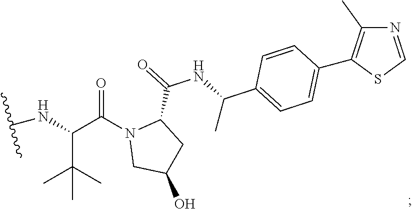 [SMILES: *N[C@H](C(=O)N1C[C@H](O)C[C@H]1C(=O)N[C@@H](C)c1ccc(-c2scnc2C)cc1)C(C)(C)C]

and L is selected from the group consisting of.

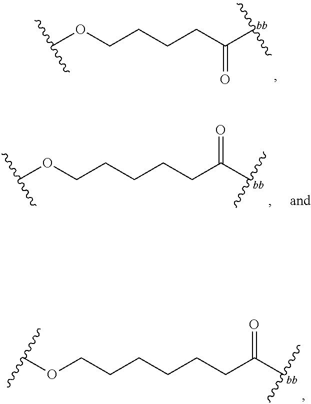

wherein bb represents the point of attachment to VBM.

In some embodiments, VBM is (V1); and L is (L-2), wherein L^{A3b} is C(═O). In some embodiments, VBM is (V1a); and L is (L-2), wherein L^{A3b} is C(═O); a3a is 1; L^{A3a} is —O—; and a1a+a1b is 1, 2, or 3. In some embodiments, VBM is

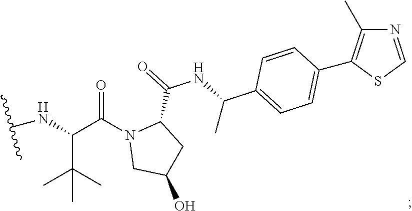 [SMILES: *N[C@H](C(=O)N1C[C@H](O)C[C@H]1C(=O)N[C@@H](C)c1ccc(-c2scnc2C)cc1)C(C)(C)C]

and L is selected from the group consisting of:

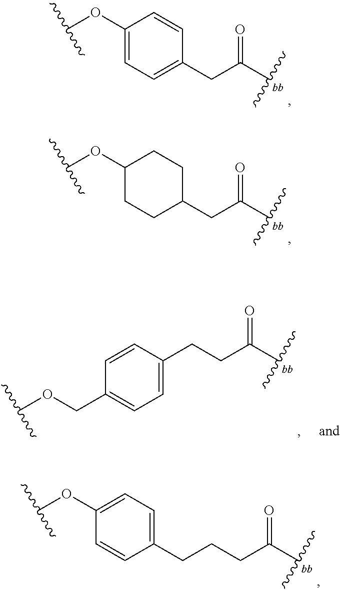

wherein bb represents the point of attachment to VBM.

In some embodiments, the compounds are selected from the group consisting of the compounds in Table C1, or pharmaceutically acceptable salts thereof.

TABLE C1

| No. | Compound Structure |
|---|---|
| 101 | 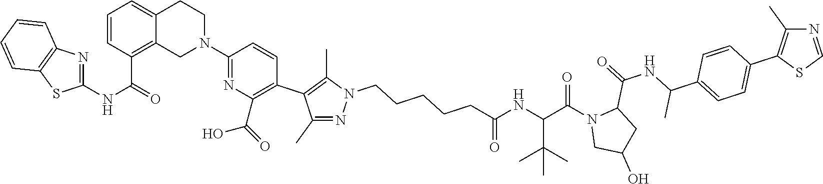 [SMILES: Cc1ncsc1-c1ccc(C(C)NC(=O)C2CC(O)CN2C(=O)C(NC(=O)CCCCCn2nc(C)c(-c3ccc(N4CCc5cccc(C(=O)Nc6nc7ccccc7s6)c5C4)nc3C(=O)O)c2C)C(C)(C)C)cc1] |
| 101a | 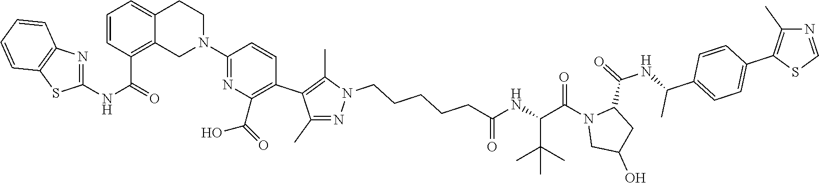 |
| 102 | 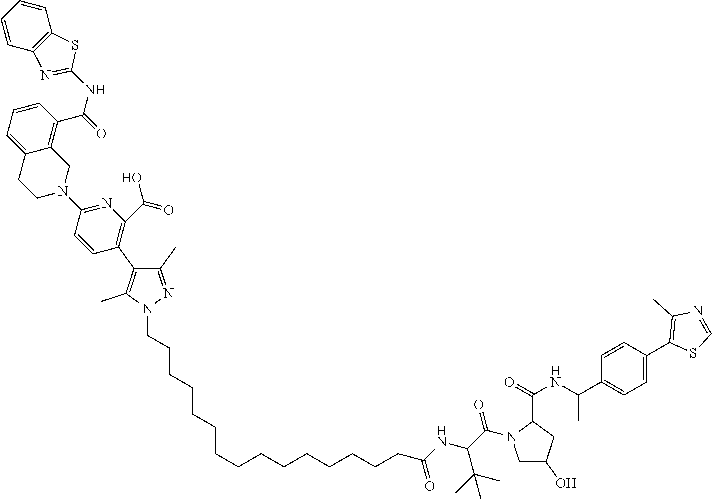 [SMILES: Cc1ncsc1-c1ccc(C(C)NC(=O)C2CC(O)CN2C(=O)C(NC(=O)CCCCCCCCCCCCCCCn2nc(C)c(-c3ccc(N4CCc5cccc(C(=O)Nc6nc7ccccc7s6)c5C4)nc3C(=O)O)c2C)C(C)(C)C)cc1] |
| 102a | 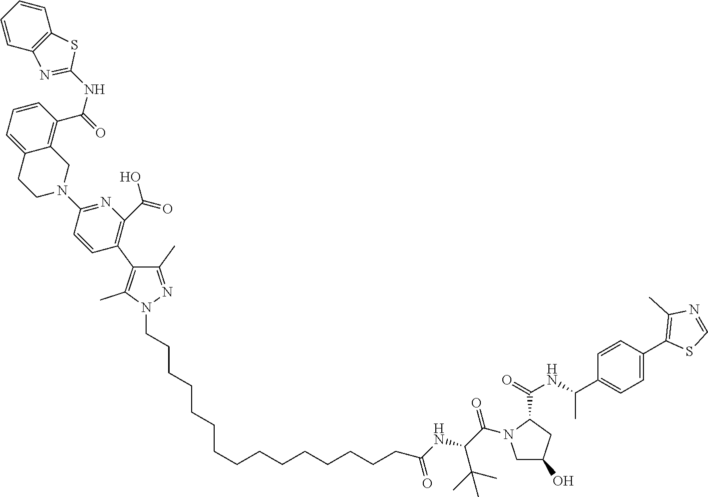 [SMILES: Cc1ncsc1-c1ccc([C@H](C)NC(=O)[C@@H]2C[C@@H](O)CN2C(=O)[C@@H](NC(=O)CCCCCCCCCCCCCCCn2nc(C)c(-c3ccc(N4CCc5cccc(C(=O)Nc6nc7ccccc7s6)c5C4)nc3C(=O)O)c2C)C(C)(C)C)cc1] |
| 103 | 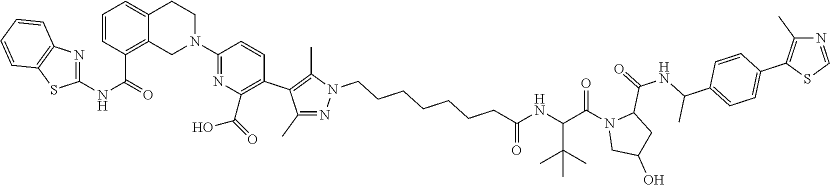 [SMILES: Cc1ncsc1-c1ccc(C(C)NC(=O)C2CC(O)CN2C(=O)C(NC(=O)CCCCCCCn2nc(C)c(-c3ccc(N4CCc5cccc(C(=O)Nc6nc7ccccc7s6)c5C4)nc3C(=O)O)c2C)C(C)(C)C)cc1] |
| 103a | 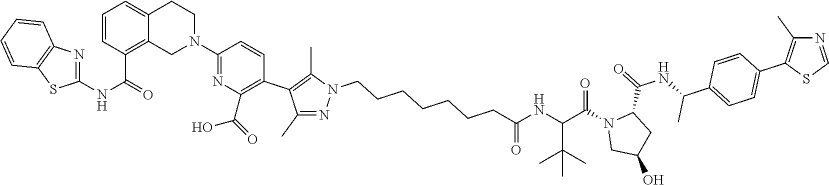 |
| 104 | 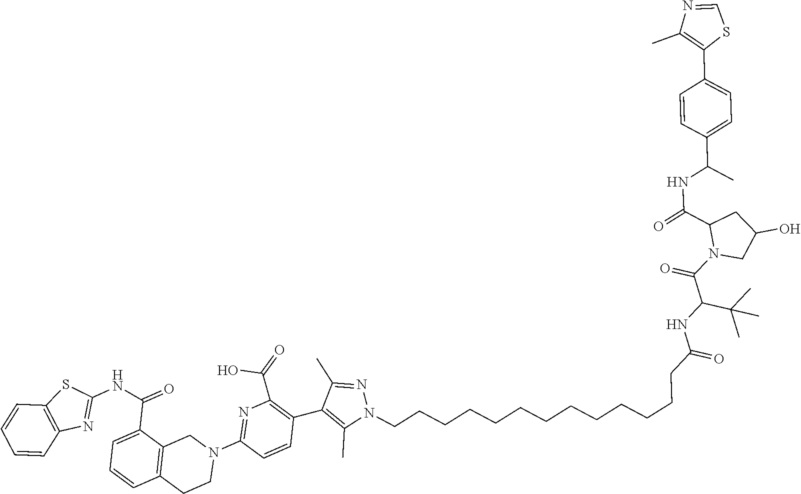 [SMILES: Cc1ncsc1-c1ccc(C(C)NC(=O)C2CC(O)CN2C(=O)C(NC(=O)CCCCCCCCCCCCCn2nc(C)c(-c3ccc(N4CCc5cccc(C(=O)Nc6nc7ccccc7s6)c5C4)nc3C(=O)O)c2C)C(C)(C)C)cc1] |
| 104a | 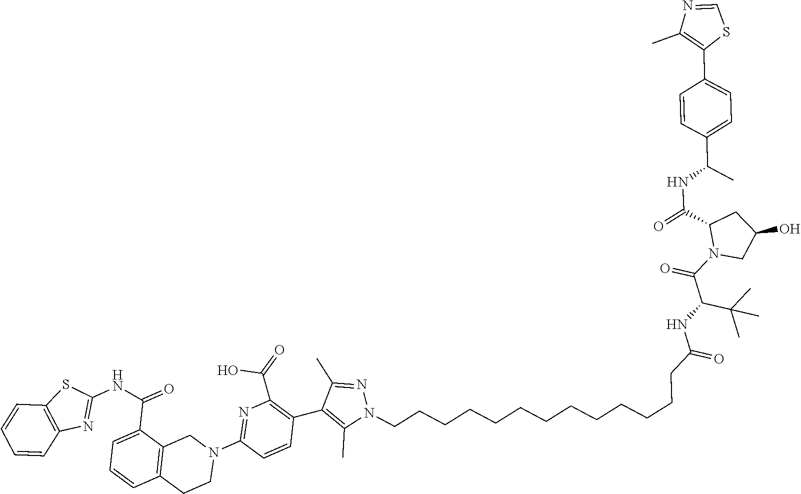 [SMILES: Cc1ncsc1-c1ccc([C@H](C)NC(=O)[C@@H]2C[C@@H](O)CN2C(=O)[C@@H](NC(=O)CCCCCCCCCCCCCn2nc(C)c(-c3ccc(N4CCc5cccc(C(=O)Nc6nc7ccccc7s6)c5C4)nc3C(=O)O)c2C)C(C)(C)C)cc1] |
| 105 | 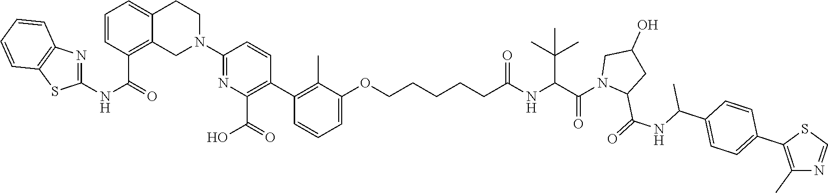 [SMILES: Cc1ncsc1-c1ccc(C(C)NC(=O)C2CC(O)CN2C(=O)C(NC(=O)CCCCCOc2cccc(-c3ccc(N4CCc5cccc(C(=O)Nc6nc7ccccc7s6)c5C4)nc3C(=O)O)c2C)C(C)(C)C)cc1] |
| 105a | 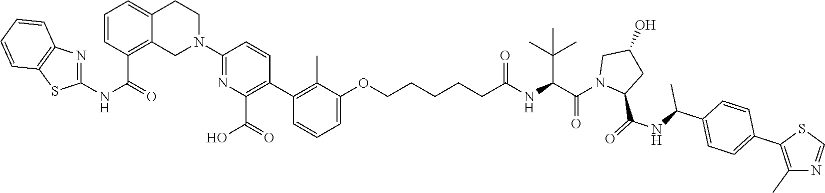 [SMILES: Cc1ncsc1-c1ccc([C@H](C)NC(=O)[C@@H]2C[C@@H](O)CN2C(=O)[C@@H](NC(=O)CCCCCOc2cccc(-c3ccc(N4CCc5cccc(C(=O)Nc6nc7ccccc7s6)c5C4)nc3C(=O)O)c2C)C(C)(C)C)cc1] |
| 106 | 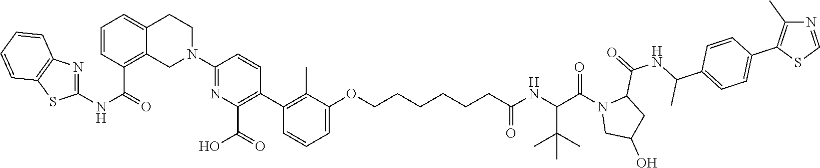 [SMILES: Cc1ncsc1-c1ccc(C(C)NC(=O)C2CC(O)CN2C(=O)C(NC(=O)CCCCCCOc2cccc(-c3ccc(N4CCc5cccc(C(=O)Nc6nc7ccccc7s6)c5C4)nc3C(=O)O)c2C)C(C)(C)C)cc1] |
| 106a | 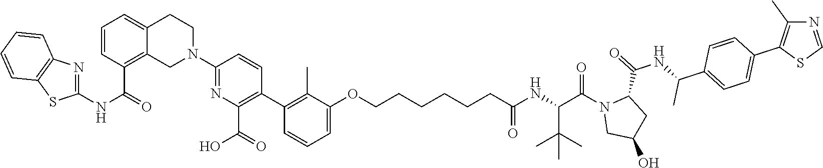 [SMILES: Cc1ncsc1-c1ccc([C@H](C)NC(=O)[C@@H]2C[C@@H](O)CN2C(=O)[C@@H](NC(=O)CCCCCCOc2cccc(-c3ccc(N4CCc5cccc(C(=O)Nc6nc7ccccc7s6)c5C4)nc3C(=O)O)c2C)C(C)(C)C)cc1] |
| 107 | 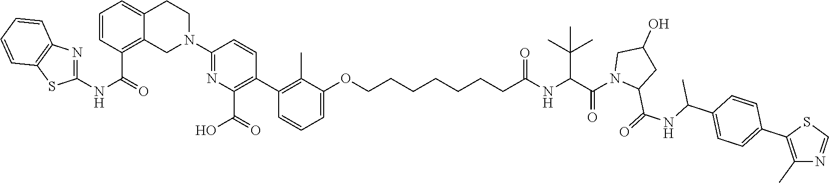 [SMILES: Cc1ncsc1-c1ccc(C(C)NC(=O)C2CC(O)CN2C(=O)C(NC(=O)CCCCCCCOc2cccc(-c3ccc(N4CCc5cccc(C(=O)Nc6nc7ccccc7s6)c5C4)nc3C(=O)O)c2C)C(C)(C)C)cc1] |
| 107a | 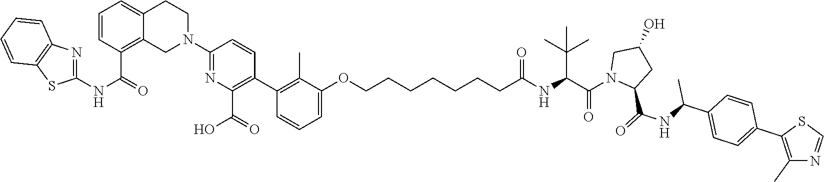 [SMILES: Cc1ncsc1-c1ccc([C@H](C)NC(=O)[C@@H]2C[C@@H](O)CN2C(=O)[C@@H](NC(=O)CCCCCCCOc2cccc(-c3ccc(N4CCc5cccc(C(=O)Nc6nc7ccccc7s6)c5C4)nc3C(=O)O)c2C)C(C)(C)C)cc1] |
| 108 | 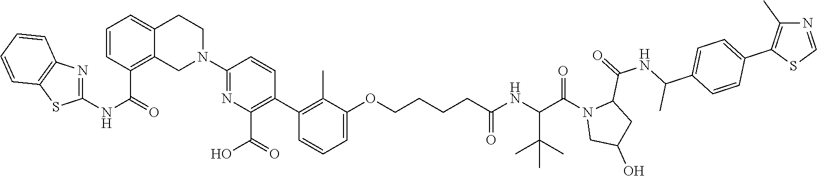 [SMILES: Cc1ncsc1-c1ccc(C(C)NC(=O)C2CC(O)CN2C(=O)C(NC(=O)CCCCOc2cccc(-c3ccc(N4CCc5cccc(C(=O)Nc6nc7ccccc7s6)c5C4)nc3C(=O)O)c2C)C(C)(C)C)cc1] |
| 108a | 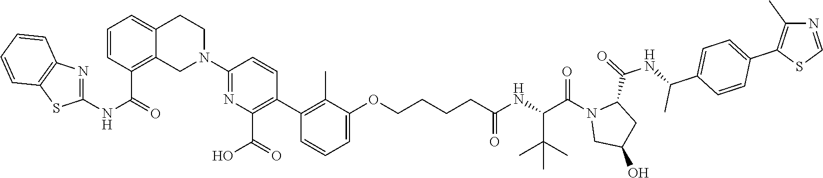 [SMILES: Cc1ncsc1-c1ccc([C@H](C)NC(=O)[C@@H]2C[C@@H](O)CN2C(=O)[C@@H](NC(=O)CCCCOc2cccc(-c3ccc(N4CCc5cccc(C(=O)Nc6nc7ccccc7s6)c5C4)nc3C(=O)O)c2C)C(C)(C)C)cc1] |
| 110 | 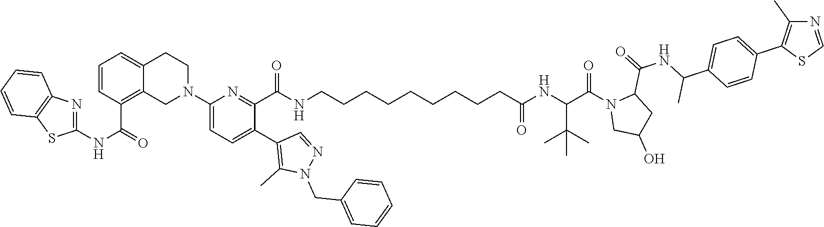 [SMILES: Cc1ncsc1-c1ccc(C(C)NC(=O)C2CC(O)CN2C(=O)C(NC(=O)CCCCCCCCCNC(=O)c2nc(N3CCc4cccc(C(=O)Nc5nc6ccccc6s5)c4C3)ccc2-c2cnn(Cc3ccccc3)c2C)C(C)(C)C)cc1] |
| 110a | 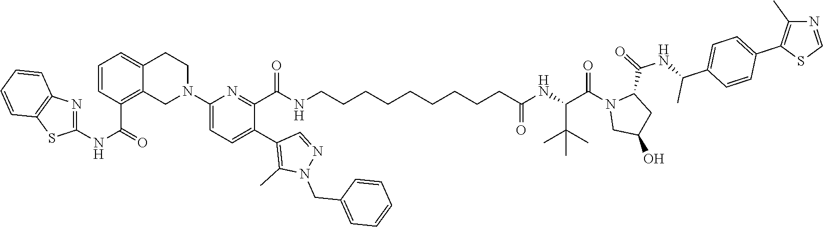 [SMILES: Cc1ncsc1-c1ccc([C@H](C)NC(=O)[C@@H]2C[C@@H](O)CN2C(=O)[C@@H](NC(=O)CCCCCCCCCNC(=O)c2nc(N3CCc4cccc(C(=O)Nc5nc6ccccc6s5)c4C3)ccc2-c2cnn(Cc3ccccc3)c2C)C(C)(C)C)cc1] |
| 111 | 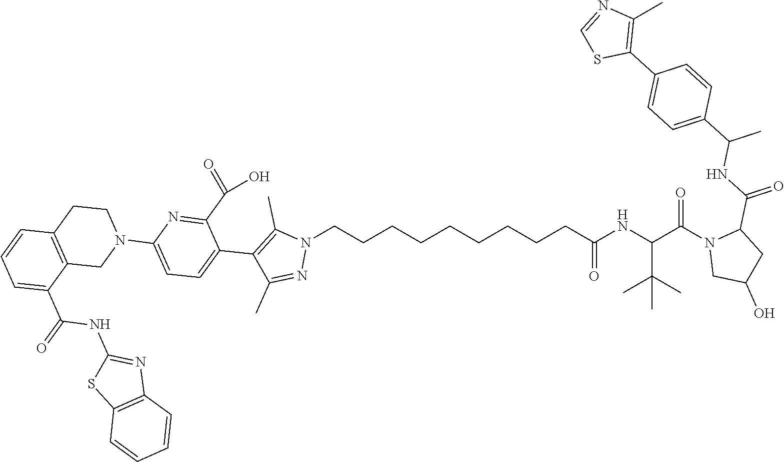 [SMILES: Cc1ncsc1-c1ccc(C(C)NC(=O)C2CC(O)CN2C(=O)C(NC(=O)CCCCCCCCCn2nc(C)c(-c3ccc(N4CCc5cccc(C(=O)Nc6nc7ccccc7s6)c5C4)nc3C(=O)O)c2C)C(C)(C)C)cc1] |
| 111a | 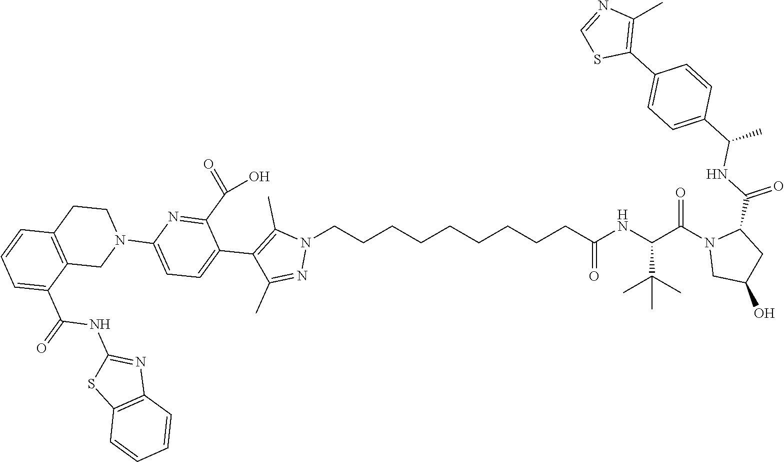 [SMILES: Cc1ncsc1-c1ccc([C@H](C)NC(=O)[C@@H]2C[C@@H](O)CN2C(=O)[C@@H](NC(=O)CCCCCCCCCn2nc(C)c(-c3ccc(N4CCc5cccc(C(=O)Nc6nc7ccccc7s6)c5C4)nc3C(=O)O)c2C)C(C)(C)C)cc1] |
| 112 | 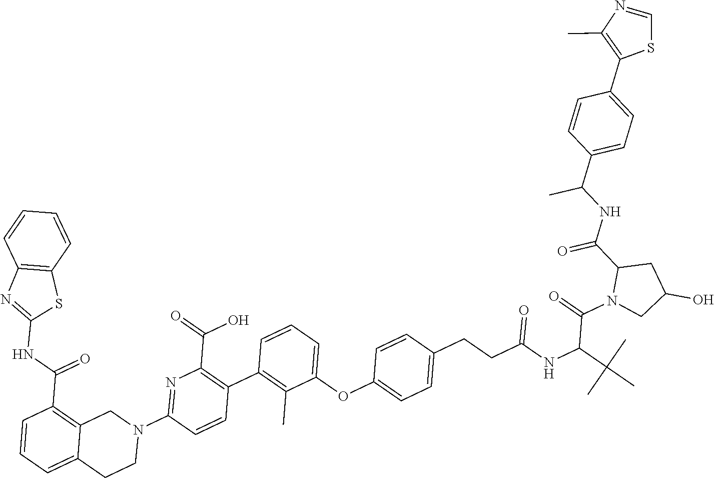 [SMILES: Cc1ncsc1-c1ccc(C(C)NC(=O)C2CC(O)CN2C(=O)C(NC(=O)CCc2ccc(Oc3cccc(-c4ccc(N5CCc6cccc(C(=O)Nc7nc8ccccc8s7)c6C5)nc4C(=O)O)c3C)cc2)C(C)(C)C)cc1] |
| 112a | 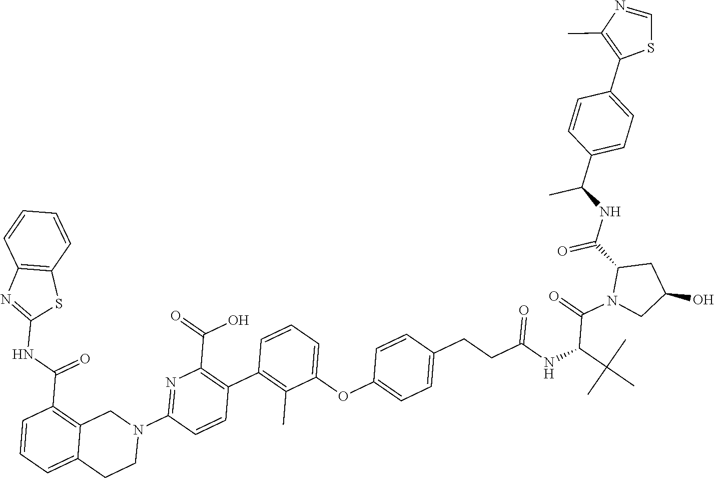 [SMILES: Cc1ncsc1-c1ccc([C@H](C)NC(=O)[C@@H]2C[C@@H](O)CN2C(=O)[C@@H](NC(=O)CCc2ccc(Oc3cccc(-c4ccc(N5CCc6cccc(C(=O)Nc7nc8ccccc8s7)c6C5)nc4C(=O)O)c3C)cc2)C(C)(C)C)cc1] |
| 113 | 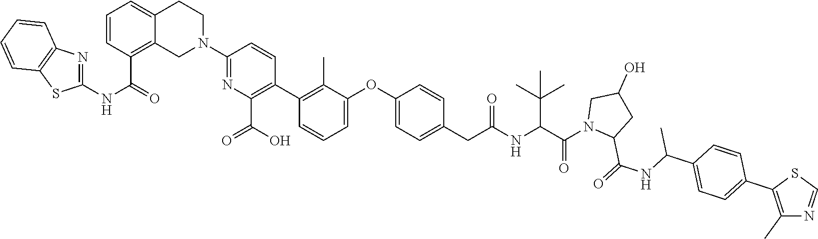 [SMILES: Cc1ncsc1-c1ccc(C(C)NC(=O)C2CC(O)CN2C(=O)C(NC(=O)Cc2ccc(Oc3cccc(-c4ccc(N5CCc6cccc(C(=O)Nc7nc8ccccc8s7)c6C5)nc4C(=O)O)c3C)cc2)C(C)(C)C)cc1] |
| 113a | 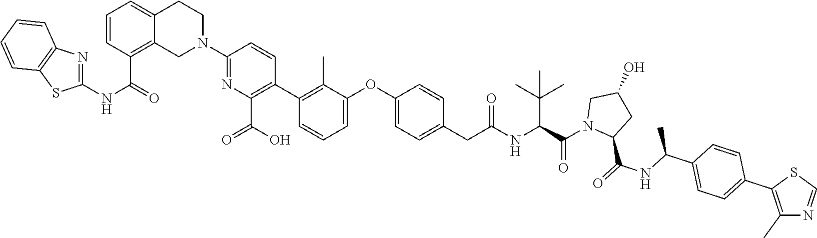 [SMILES: Cc1ncsc1-c1ccc([C@H](C)NC(=O)[C@@H]2C[C@@H](O)CN2C(=O)[C@@H](NC(=O)Cc2ccc(Oc3cccc(-c4ccc(N5CCc6cccc(C(=O)Nc7nc8ccccc8s7)c6C5)nc4C(=O)O)c3C)cc2)C(C)(C)C)cc1] |
| 114 | 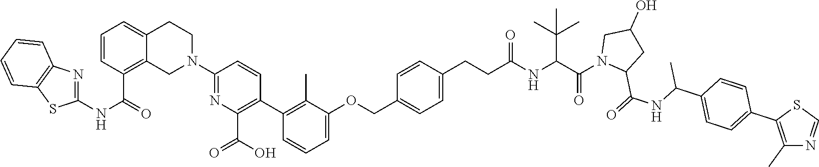 [SMILES: Cc1ncsc1-c1ccc(C(C)NC(=O)C2CC(O)CN2C(=O)C(NC(=O)CCc2ccc(COc3cccc(-c4ccc(N5CCc6cccc(C(=O)Nc7nc8ccccc8s7)c6C5)nc4C(=O)O)c3C)cc2)C(C)(C)C)cc1] |
| 114a | 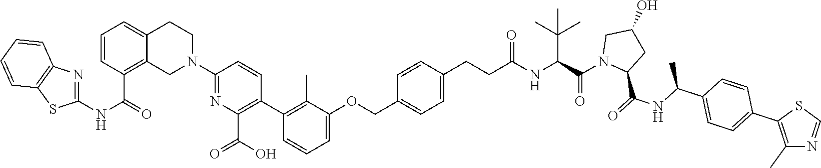 [SMILES: Cc1ncsc1-c1ccc([C@H](C)NC(=O)[C@@H]2C[C@@H](O)CN2C(=O)[C@@H](NC(=O)CCc2ccc(COc3cccc(-c4ccc(N5CCc6cccc(C(=O)Nc7nc8ccccc8s7)c6C5)nc4C(=O)O)c3C)cc2)C(C)(C)C)cc1] |
| 115 | 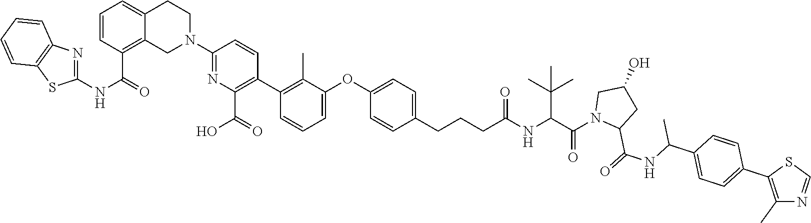 [SMILES: Cc1ncsc1-c1ccc(C(C)NC(=O)C2C[C@@H](O)CN2C(=O)C(NC(=O)CCCc2ccc(Oc3cccc(-c4ccc(N5CCc6cccc(C(=O)Nc7nc8ccccc8s7)c6C5)nc4C(=O)O)c3C)cc2)C(C)(C)C)cc1] |
| 115a | 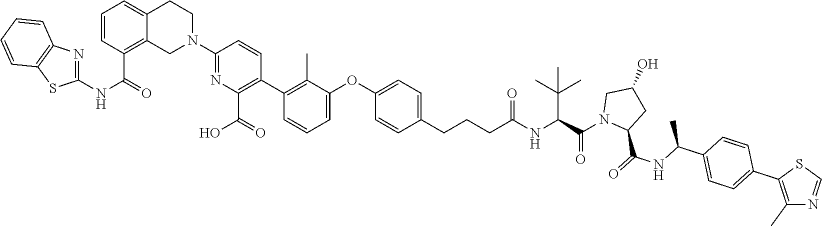 [SMILES: Cc1ncsc1-c1ccc([C@H](C)NC(=O)[C@@H]2C[C@@H](O)CN2C(=O)[C@@H](NC(=O)CCCc2ccc(Oc3cccc(-c4ccc(N5CCc6cccc(C(=O)Nc7nc8ccccc8s7)c6C5)nc4C(=O)O)c3C)cc2)C(C)(C)C)cc1] |
| 116 | 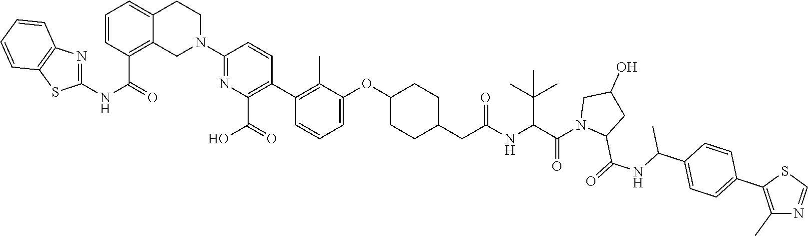 [SMILES: Cc1ncsc1-c1ccc(C(C)NC(=O)C2CC(O)CN2C(=O)C(NC(=O)CC2CCC(Oc3cccc(-c4ccc(N5CCc6cccc(C(=O)Nc7nc8ccccc8s7)c6C5)nc4C(=O)O)c3C)CC2)C(C)(C)C)cc1] |
| 116a | 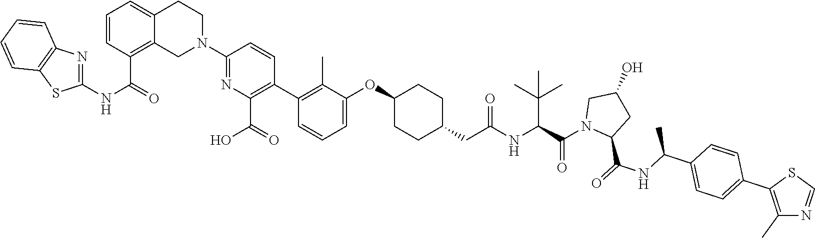 [SMILES: Cc1ncsc1-c1ccc([C@H](C)NC(=O)[C@@H]2C[C@@H](O)CN2C(=O)[C@@H](NC(=O)C[C@H]2CC[C@H](Oc3cccc(-c4ccc(N5CCc6cccc(C(=O)Nc7nc8ccccc8s7)c6C5)nc4C(=O)O)c3C)CC2)C(C)(C)C)cc1] |
| 116b | 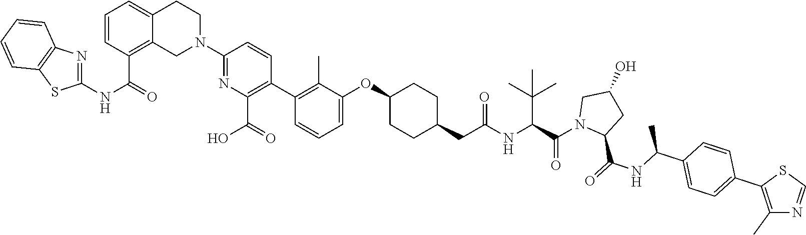 [SMILES: Cc1ncsc1-c1ccc([C@H](C)NC(=O)[C@@H]2C[C@@H](O)CN2C(=O)[C@@H](NC(=O)C[C@H]2CC[C@@H](Oc3cccc(-c4ccc(N5CCc6cccc(C(=O)Nc7nc8ccccc8s7)c6C5)nc4C(=O)O)c3C)CC2)C(C)(C)C)cc1] |
| 117 | 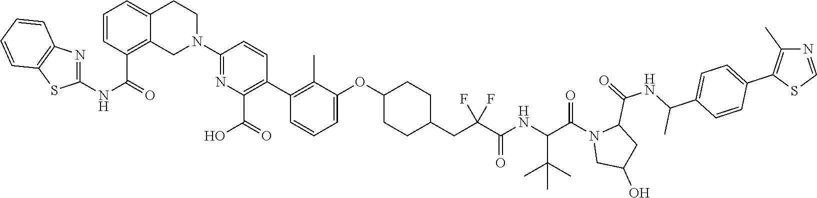 [SMILES: Cc1ncsc1-c1ccc(C(C)NC(=O)C2CC(O)CN2C(=O)C(NC(=O)C(F)(F)CC2CCC(Oc3cccc(-c4ccc(N5CCc6cccc(C(=O)Nc7nc8ccccc8s7)c6C5)nc4C(=O)O)c3C)CC2)C(C)(C)C)cc1] |
| 117a | 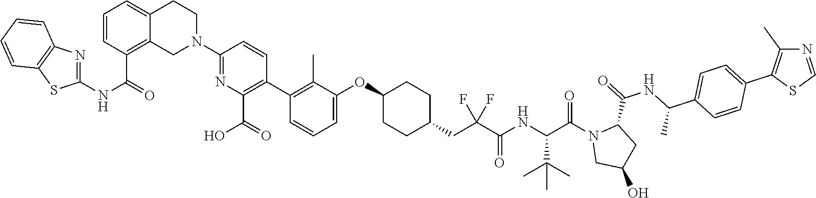 [SMILES: Cc1ncsc1-c1ccc([C@H](C)NC(=O)[C@@H]2C[C@@H](O)CN2C(=O)[C@@H](NC(=O)C(F)(F)C[C@H]2CC[C@H](Oc3cccc(-c4ccc(N5CCc6cccc(C(=O)Nc7nc8ccccc8s7)c6C5)nc4C(=O)O)c3C)CC2)C(C)(C)C)cc1] |
| 118 | 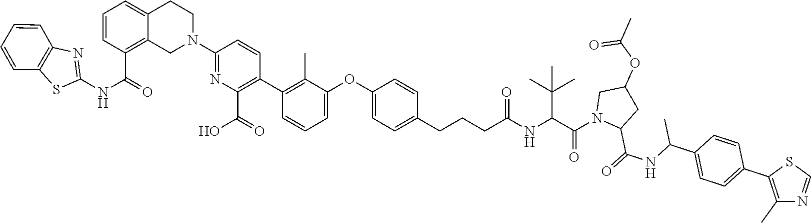 [SMILES: CC(=O)OC1CC(C(=O)NC(C)c2ccc(-c3scnc3C)cc2)N(C(=O)C(NC(=O)CCCc2ccc(Oc3cccc(-c4ccc(N5CCc6cccc(C(=O)Nc7nc8ccccc8s7)c6C5)nc4C(=O)O)c3C)cc2)C(C)(C)C)C1] |
| 118a | 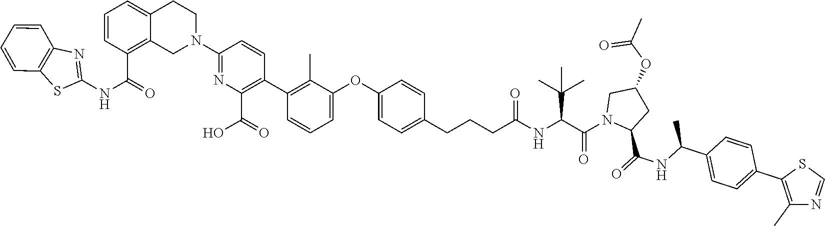 [SMILES: CC(=O)O[C@@H]1C[C@@H](C(=O)N[C@@H](C)c2ccc(-c3scnc3C)cc2)N(C(=O)[C@@H](NC(=O)CCCc2ccc(Oc3cccc(-c4ccc(N5CCc6cccc(C(=O)Nc7nc8ccccc8s7)c6C5)nc4C(=O)O)c3C)cc2)C(C)(C)C)C1] |
| 119 | 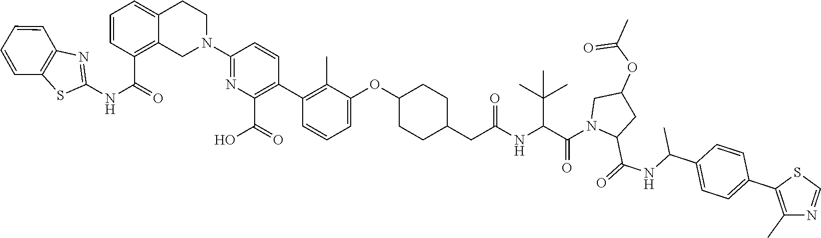 [SMILES: CC(=O)OC1CC(C(=O)NC(C)c2ccc(-c3scnc3C)cc2)N(C(=O)C(NC(=O)CC2CCC(Oc3cccc(-c4ccc(N5CCc6cccc(C(=O)Nc7nc8ccccc8s7)c6C5)nc4C(=O)O)c3C)CC2)C(C)(C)C)C1] |
| 119a | 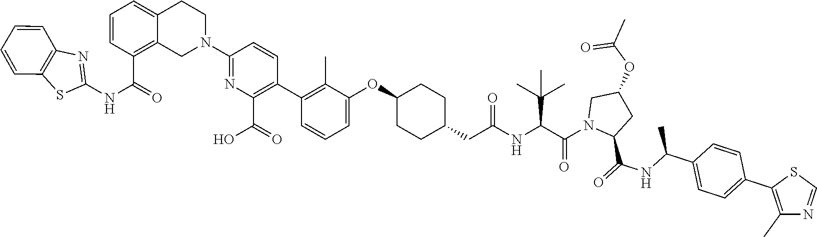 [SMILES: CC(=O)O[C@@H]1C[C@@H](C(=O)N[C@@H](C)c2ccc(-c3scnc3C)cc2)N(C(=O)[C@@H](NC(=O)C[C@H]2CC[C@H](Oc3cccc(-c4ccc(N5CCc6cccc(C(=O)Nc7nc8ccccc8s7)c6C5)nc4C(=O)O)c3C)CC2)C(C)(C)C)C1] |
| 120 | 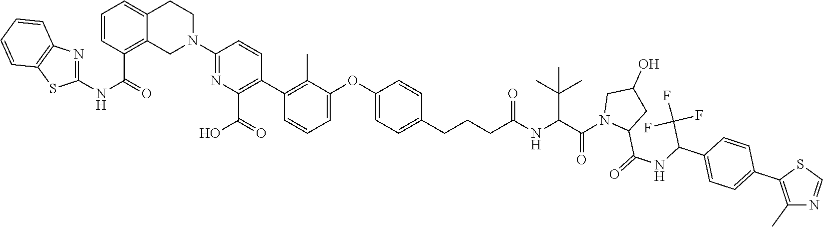 [SMILES: Cc1ncsc1-c1ccc(C(NC(=O)C2CC(O)CN2C(=O)C(NC(=O)CCCc2ccc(Oc3cccc(-c4ccc(N5CCc6cccc(C(=O)Nc7nc8ccccc8s7)c6C5)nc4C(=O)O)c3C)cc2)C(C)(C)C)C(F)(F)F)cc1] |
| 120a | 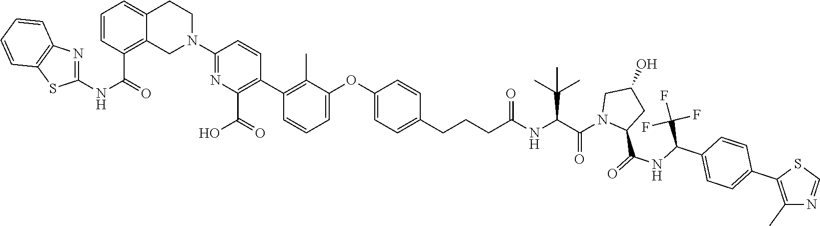 [SMILES: Cc1ncsc1-c1ccc([C@@H](NC(=O)[C@@H]2C[C@@H](O)CN2C(=O)[C@@H](NC(=O)CCCc2ccc(Oc3cccc(-c4ccc(N5CCc6cccc(C(=O)Nc7nc8ccccc8s7)c6C5)nc4C(=O)O)c3C)cc2)C(C)(C)C)C(F)(F)F)cc1] |
| 121 | 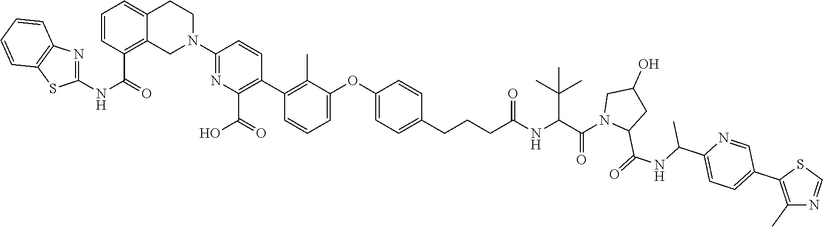 [SMILES: Cc1ncsc1-c1ccc(C(C)NC(=O)C2CC(O)CN2C(=O)C(NC(=O)CCCc2ccc(Oc3cccc(-c4ccc(N5CCc6cccc(C(=O)Nc7nc8ccccc8s7)c6C5)nc4C(=O)O)c3C)cc2)C(C)(C)C)nc1] |
| 121a | 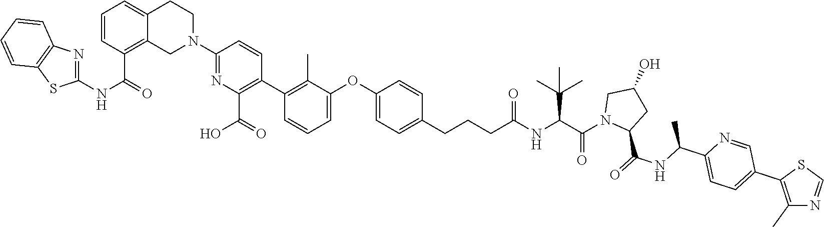 [SMILES: Cc1ncsc1-c1ccc([C@H](C)NC(=O)[C@@H]2C[C@@H](O)CN2C(=O)[C@@H](NC(=O)CCCc2ccc(Oc3cccc(-c4ccc(N5CCc6cccc(C(=O)Nc7nc8ccccc8s7)c6C5)nc4C(=O)O)c3C)cc2)C(C)(C)C)nc1] |
| 122 | 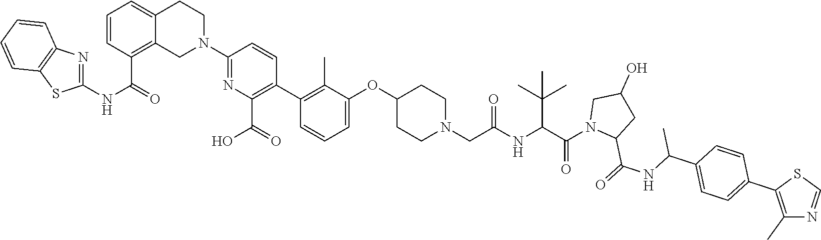 [SMILES: Cc1ncsc1-c1ccc(C(C)NC(=O)C2CC(O)CN2C(=O)C(NC(=O)CN2CCC(Oc3cccc(-c4ccc(N5CCc6cccc(C(=O)Nc7nc8ccccc8s7)c6C5)nc4C(=O)O)c3C)CC2)C(C)(C)C)cc1] |
| 122a | 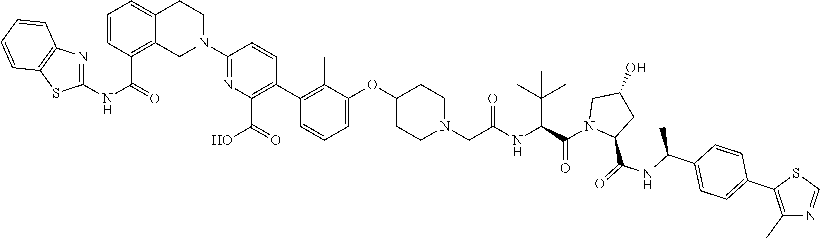 [SMILES: Cc1ncsc1-c1ccc([C@H](C)NC(=O)[C@@H]2C[C@@H](O)CN2C(=O)[C@@H](NC(=O)CN2CCC(Oc3cccc(-c4ccc(N5CCc6cccc(C(=O)Nc7nc8ccccc8s7)c6C5)nc4C(=O)O)c3C)CC2)C(C)(C)C)cc1] |
| 123 | 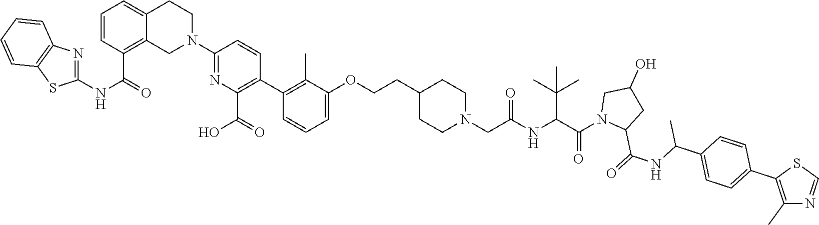 [SMILES: Cc1ncsc1-c1ccc(C(C)NC(=O)C2CC(O)CN2C(=O)C(NC(=O)CN2CCC(CCOc3cccc(-c4ccc(N5CCc6cccc(C(=O)Nc7nc8ccccc8s7)c6C5)nc4C(=O)O)c3C)CC2)C(C)(C)C)cc1] |
| 123a | 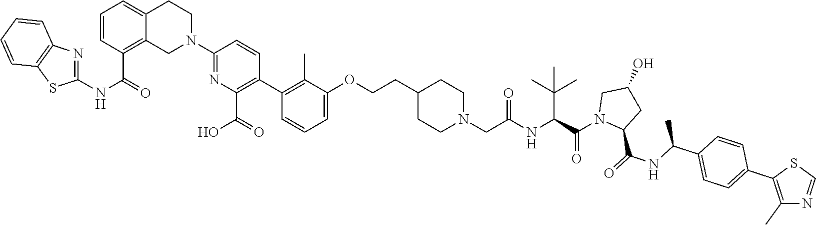 [SMILES: Cc1ncsc1-c1ccc([C@H](C)NC(=O)[C@@H]2C[C@@H](O)CN2C(=O)[C@@H](NC(=O)CN2CCC(CCOc3cccc(-c4ccc(N5CCc6cccc(C(=O)Nc7nc8ccccc8s7)c6C5)nc4C(=O)O)c3C)CC2)C(C)(C)C)cc1] |
| 124 | 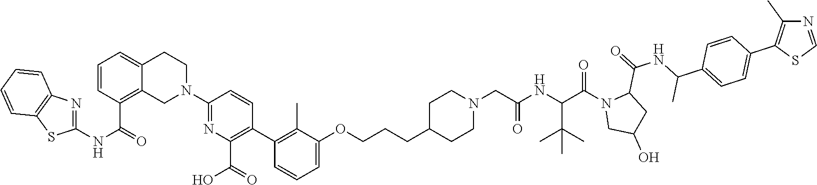 [SMILES: Cc1ncsc1-c1ccc(C(C)NC(=O)C2CC(O)CN2C(=O)C(NC(=O)CN2CCC(CCCOc3cccc(-c4ccc(N5CCc6cccc(C(=O)Nc7nc8ccccc8s7)c6C5)nc4C(=O)O)c3C)CC2)C(C)(C)C)cc1] |
| 124a | 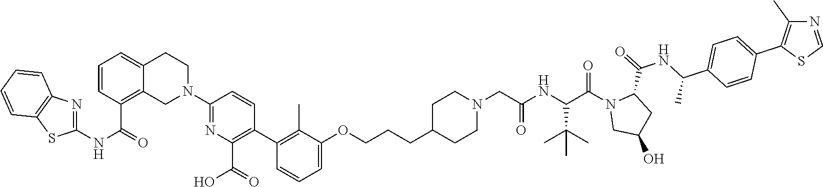 |
| 125 | 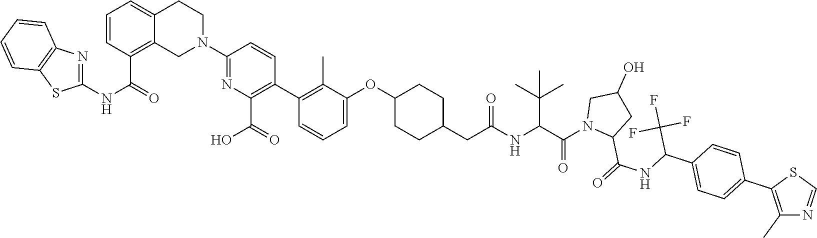 [SMILES: Cc1ncsc1-c1ccc(C(NC(=O)C2CC(O)CN2C(=O)C(NC(=O)CC2CCC(Oc3cccc(-c4ccc(N5CCc6cccc(C(=O)Nc7nc8ccccc8s7)c6C5)nc4C(=O)O)c3C)CC2)C(C)(C)C)C(F)(F)F)cc1] |
| 125a | 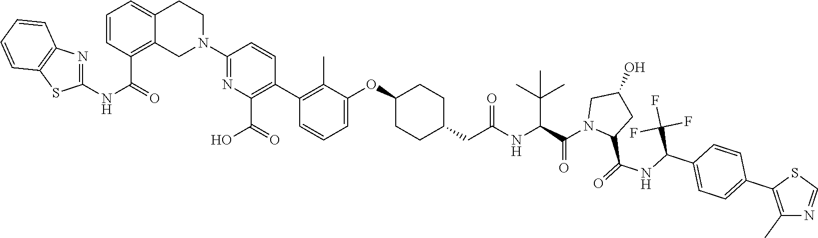 |
| 126 | 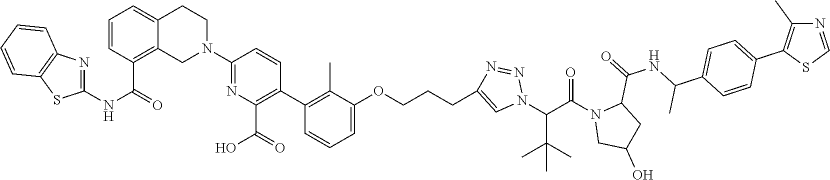 [SMILES: Cc1ncsc1-c1ccc(C(C)NC(=O)C2CC(O)CN2C(=O)C(n2cc(CCCOc3cccc(-c4ccc(N5CCc6cccc(C(=O)Nc7nc8ccccc8s7)c6C5)nc4C(=O)O)c3C)nn2)C(C)(C)C)cc1] |
| 126a | 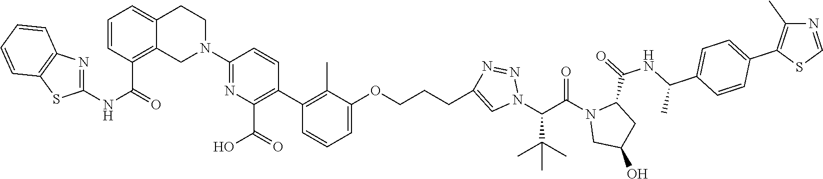 [SMILES: Cc1ncsc1-c1ccc([C@H](C)NC(=O)[C@@H]2C[C@@H](O)CN2C(=O)[C@@H](n2cc(CCCOc3cccc(-c4ccc(N5CCc6cccc(C(=O)Nc7nc8ccccc8s7)c6C5)nc4C(=O)O)c3C)nn2)C(C)(C)C)cc1] |
| 127 | 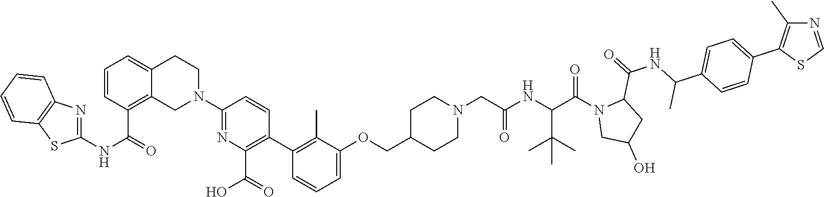 [SMILES: Cc1ncsc1-c1ccc(C(C)NC(=O)C2CC(O)CN2C(=O)C(NC(=O)CN2CCC(COc3cccc(-c4ccc(N5CCc6cccc(C(=O)Nc7nc8ccccc8s7)c6C5)nc4C(=O)O)c3C)CC2)C(C)(C)C)cc1] |
| 127a | 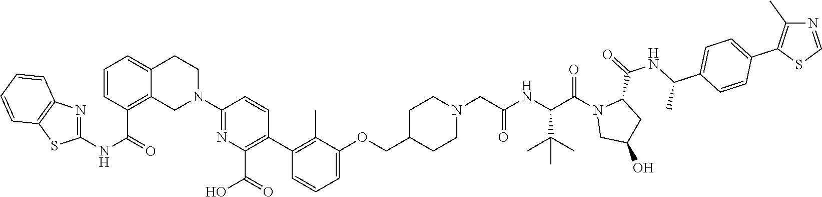 [SMILES: Cc1ncsc1-c1ccc([C@H](C)NC(=O)[C@@H]2C[C@@H](O)CN2C(=O)[C@@H](NC(=O)CN2CCC(COc3cccc(-c4ccc(N5CCc6cccc(C(=O)Nc7nc8ccccc8s7)c6C5)nc4C(=O)O)c3C)CC2)C(C)(C)C)cc1] |
| 128 | 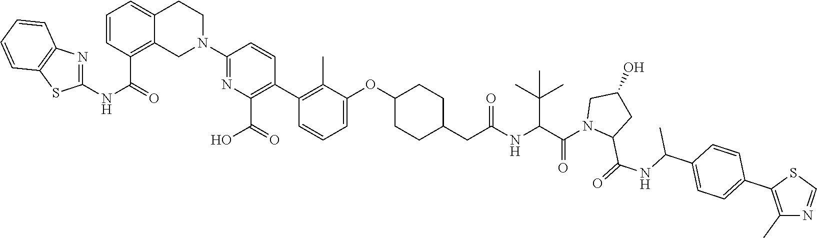 [SMILES: Cc1ncsc1-c1ccc(C(C)NC(=O)C2C[C@@H](O)CN2C(=O)C(NC(=O)CC2CCC(Oc3cccc(-c4ccc(N5CCc6cccc(C(=O)Nc7nc8ccccc8s7)c6C5)nc4C(=O)O)c3C)CC2)C(C)(C)C)cc1] |
| 128a | 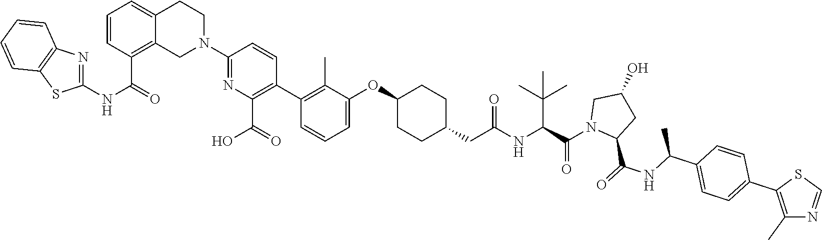 [SMILES: Cc1ncsc1-c1ccc([C@H](C)NC(=O)[C@@H]2C[C@@H](O)CN2C(=O)[C@@H](NC(=O)C[C@H]2CC[C@H](Oc3cccc(-c4ccc(N5CCc6cccc(C(=O)Nc7nc8ccccc8s7)c6C5)nc4C(=O)O)c3C)CC2)C(C)(C)C)cc1] |
| 129 | 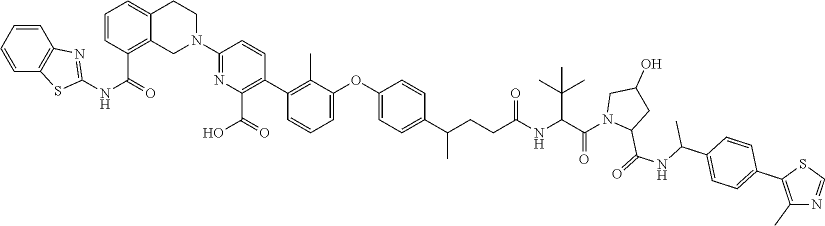 [SMILES: Cc1ncsc1-c1ccc(C(C)NC(=O)C2CC(O)CN2C(=O)C(NC(=O)CCC(C)c2ccc(Oc3cccc(-c4ccc(N5CCc6cccc(C(=O)Nc7nc8ccccc8s7)c6C5)nc4C(=O)O)c3C)cc2)C(C)(C)C)cc1] |
| 129a | 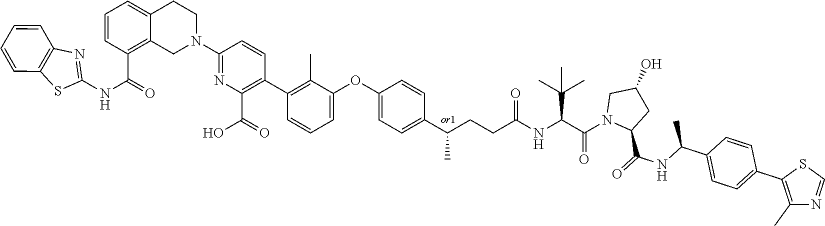 [SMILES: Cc1ncsc1-c1ccc([C@H](C)NC(=O)[C@@H]2C[C@@H](O)CN2C(=O)[C@@H](NC(=O)CC[C@H](C)c2ccc(Oc3cccc(-c4ccc(N5CCc6cccc(C(=O)Nc7nc8ccccc8s7)c6C5)nc4C(=O)O)c3C)cc2)C(C)(C)C)cc1] |
| 129b | 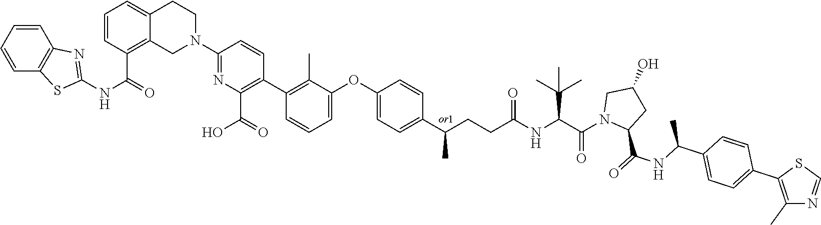 [SMILES: Cc1ncsc1-c1ccc([C@H](C)NC(=O)[C@@H]2C[C@@H](O)CN2C(=O)[C@@H](NC(=O)CC[C@@H](C)c2ccc(Oc3cccc(-c4ccc(N5CCc6cccc(C(=O)Nc7nc8ccccc8s7)c6C5)nc4C(=O)O)c3C)cc2)C(C)(C)C)cc1] |
| 130 | 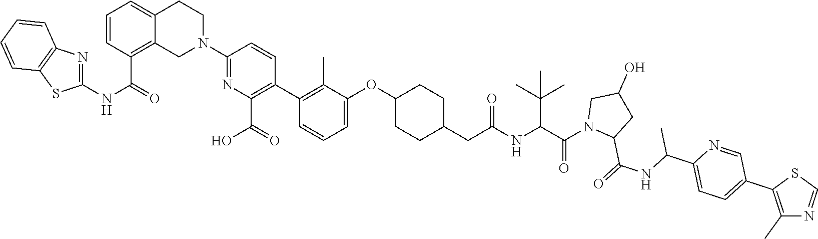 [SMILES: Cc1ncsc1-c1ccc(C(C)NC(=O)C2CC(O)CN2C(=O)C(NC(=O)CC2CCC(Oc3cccc(-c4ccc(N5CCc6cccc(C(=O)Nc7nc8ccccc8s7)c6C5)nc4C(=O)O)c3C)CC2)C(C)(C)C)nc1] |
| 130a | 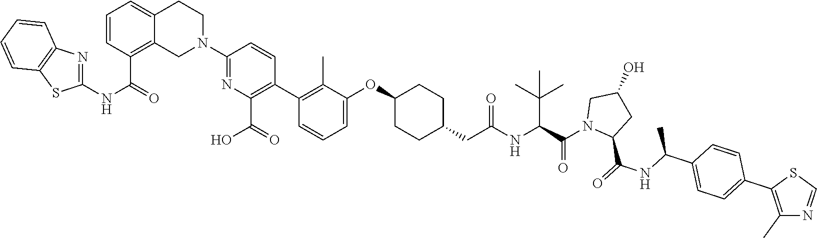 [SMILES: Cc1ncsc1-c1ccc([C@H](C)NC(=O)[C@@H]2C[C@@H](O)CN2C(=O)[C@@H](NC(=O)C[C@H]2CC[C@H](Oc3cccc(-c4ccc(N5CCc6cccc(C(=O)Nc7nc8ccccc8s7)c6C5)nc4C(=O)O)c3C)CC2)C(C)(C)C)cc1] |
| 131 | 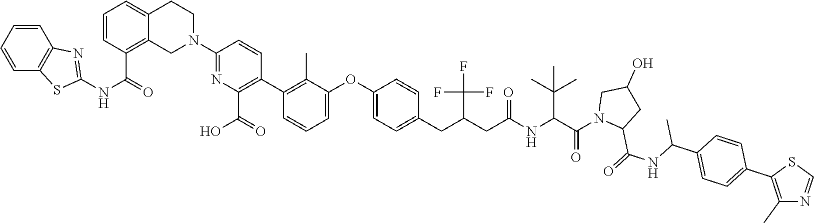 [SMILES: Cc1ncsc1-c1ccc(C(C)NC(=O)C2CC(O)CN2C(=O)C(NC(=O)CC(Cc2ccc(Oc3cccc(-c4ccc(N5CCc6cccc(C(=O)Nc7nc8ccccc8s7)c6C5)nc4C(=O)O)c3C)cc2)C(F)(F)F)C(C)(C)C)cc1] |
| 131a | 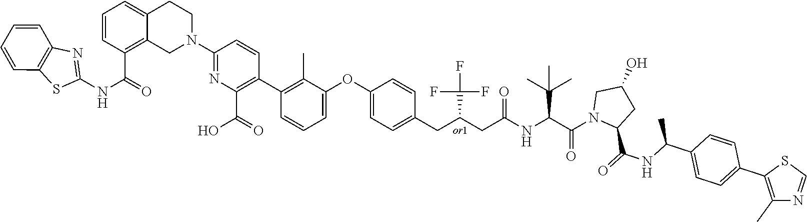 [SMILES: Cc1ncsc1-c1ccc([C@H](C)NC(=O)[C@@H]2C[C@@H](O)CN2C(=O)[C@@H](NC(=O)C[C@H](Cc2ccc(Oc3cccc(-c4ccc(N5CCc6cccc(C(=O)Nc7nc8ccccc8s7)c6C5)nc4C(=O)O)c3C)cc2)C(F)(F)F)C(C)(C)C)cc1] |
| 131b | 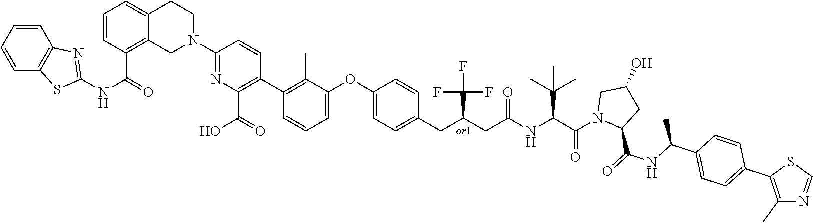 [SMILES: Cc1ncsc1-c1ccc([C@H](C)NC(=O)[C@@H]2C[C@@H](O)CN2C(=O)[C@@H](NC(=O)C[C@@H](Cc2ccc(Oc3cccc(-c4ccc(N5CCc6cccc(C(=O)Nc7nc8ccccc8s7)c6C5)nc4C(=O)O)c3C)cc2)C(F)(F)F)C(C)(C)C)cc1] |
| 132 | 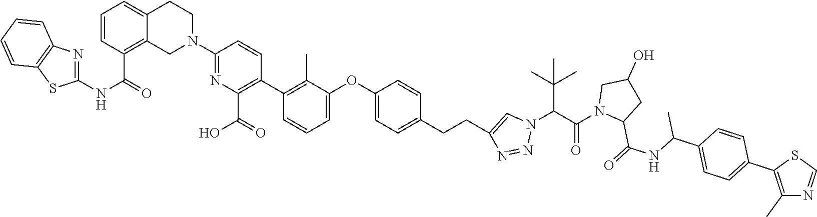 [SMILES: Cc1ncsc1-c1ccc(C(C)NC(=O)C2CC(O)CN2C(=O)C(n2cc(CCc3ccc(Oc4cccc(-c5ccc(N6CCc7cccc(C(=O)Nc8nc9ccccc9s8)c7C6)nc5C(=O)O)c4C)cc3)nn2)C(C)(C)C)cc1] |
| 132a | 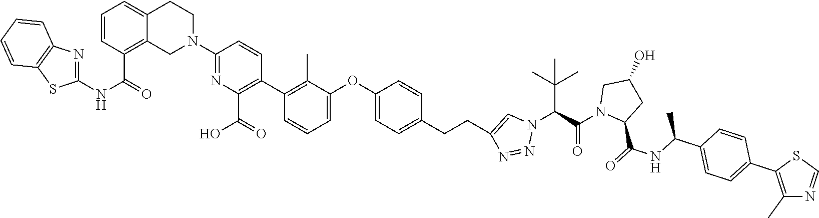 [SMILES: Cc1ncsc1-c1ccc([C@H](C)NC(=O)[C@@H]2C[C@@H](O)CN2C(=O)[C@@H](n2cc(CCc3ccc(Oc4cccc(-c5ccc(N6CCc7cccc(C(=O)Nc8nc9ccccc9s8)c7C6)nc5C(=O)O)c4C)cc3)nn2)C(C)(C)C)cc1] |
| 133 | 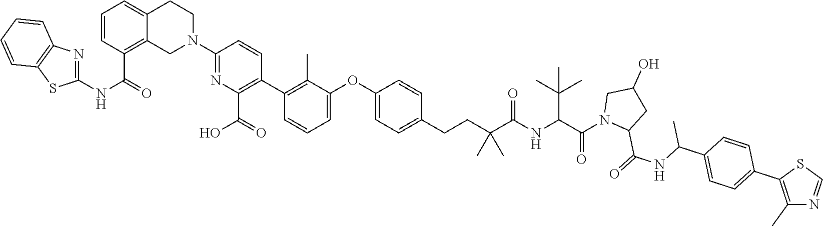 [SMILES: Cc1ncsc1-c1ccc(C(C)NC(=O)C2CC(O)CN2C(=O)C(NC(=O)C(C)(C)CCc2ccc(Oc3cccc(-c4ccc(N5CCc6cccc(C(=O)Nc7nc8ccccc8s7)c6C5)nc4C(=O)O)c3C)cc2)C(C)(C)C)cc1] |
| 133a | 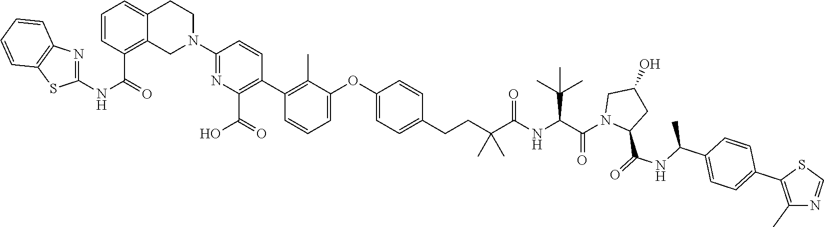 [SMILES: Cc1ncsc1-c1ccc([C@H](C)NC(=O)[C@@H]2C[C@@H](O)CN2C(=O)[C@@H](NC(=O)C(C)(C)CCc2ccc(Oc3cccc(-c4ccc(N5CCc6cccc(C(=O)Nc7nc8ccccc8s7)c6C5)nc4C(=O)O)c3C)cc2)C(C)(C)C)cc1] |
| 134 | 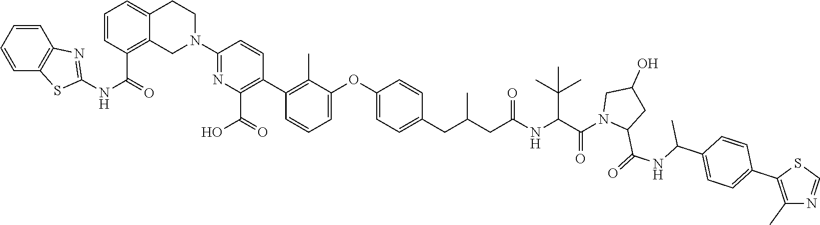 [SMILES: Cc1ncsc1-c1ccc(C(C)NC(=O)C2CC(O)CN2C(=O)C(NC(=O)CC(C)Cc2ccc(Oc3cccc(-c4ccc(N5CCc6cccc(C(=O)Nc7nc8ccccc8s7)c6C5)nc4C(=O)O)c3C)cc2)C(C)(C)C)cc1] |
| 134a | 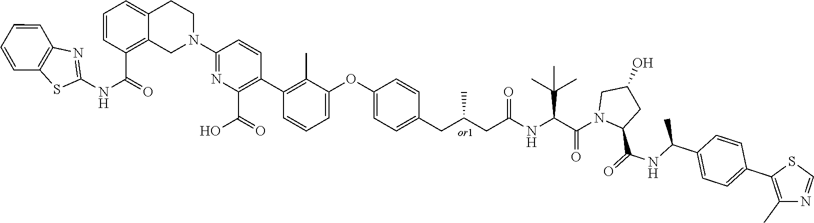 [SMILES: Cc1ncsc1-c1ccc([C@H](C)NC(=O)[C@@H]2C[C@@H](O)CN2C(=O)[C@@H](NC(=O)C[C@@H](C)Cc2ccc(Oc3cccc(-c4ccc(N5CCc6cccc(C(=O)Nc7nc8ccccc8s7)c6C5)nc4C(=O)O)c3C)cc2)C(C)(C)C)cc1] |
| 134b | 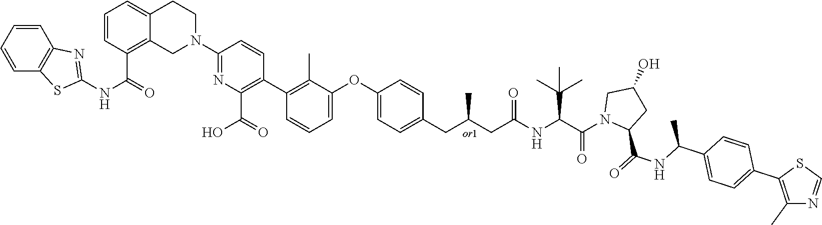 [SMILES: Cc1ncsc1-c1ccc([C@H](C)NC(=O)[C@@H]2C[C@@H](O)CN2C(=O)[C@@H](NC(=O)C[C@H](C)Cc2ccc(Oc3cccc(-c4ccc(N5CCc6cccc(C(=O)Nc7nc8ccccc8s7)c6C5)nc4C(=O)O)c3C)cc2)C(C)(C)C)cc1] |
| 135 | 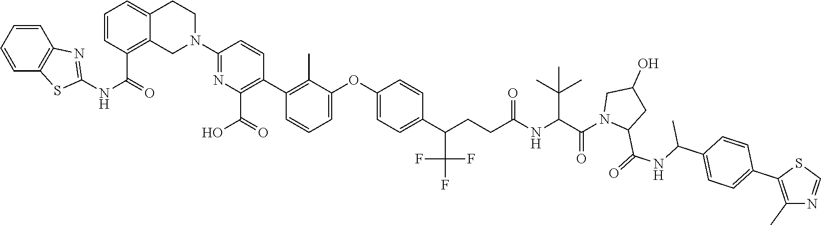 [SMILES: Cc1ncsc1-c1ccc(C(C)NC(=O)C2CC(O)CN2C(=O)C(NC(=O)CCC(c2ccc(Oc3cccc(-c4ccc(N5CCc6cccc(C(=O)Nc7nc8ccccc8s7)c6C5)nc4C(=O)O)c3C)cc2)C(F)(F)F)C(C)(C)C)cc1] |
| 135a | 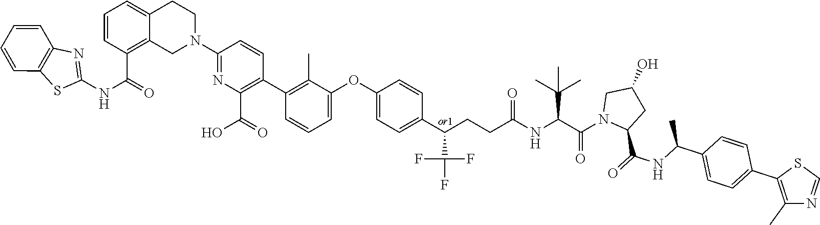 [SMILES: Cc1ncsc1-c1ccc([C@H](C)NC(=O)[C@@H]2C[C@@H](O)CN2C(=O)[C@@H](NC(=O)CC[C@@H](c2ccc(Oc3cccc(-c4ccc(N5CCc6cccc(C(=O)Nc7nc8ccccc8s7)c6C5)nc4C(=O)O)c3C)cc2)C(F)(F)F)C(C)(C)C)cc1] |
| 135b | 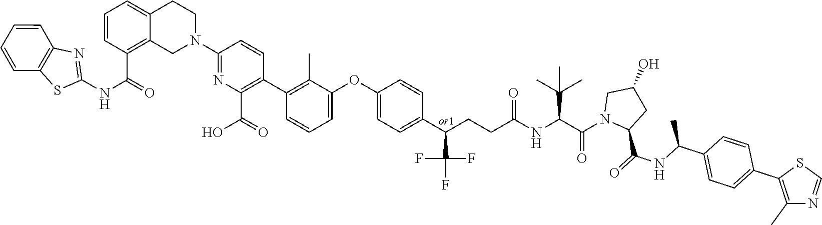 [SMILES: Cc1ncsc1-c1ccc([C@H](C)NC(=O)[C@@H]2C[C@@H](O)CN2C(=O)[C@@H](NC(=O)CC[C@H](c2ccc(Oc3cccc(-c4ccc(N5CCc6cccc(C(=O)Nc7nc8ccccc8s7)c6C5)nc4C(=O)O)c3C)cc2)C(F)(F)F)C(C)(C)C)cc1] |
| 136 | 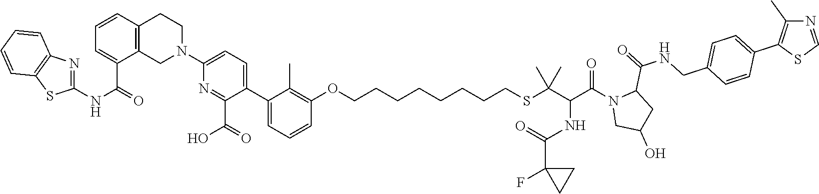 [SMILES: Cc1ncsc1-c1ccc(CNC(=O)C2CC(O)CN2C(=O)C(NC(=O)C2(F)CC2)C(C)(C)SCCCCCCCCOc2cccc(-c3ccc(N4CCc5cccc(C(=O)Nc6nc7ccccc7s6)c5C4)nc3C(=O)O)c2C)cc1] |
| 136a | 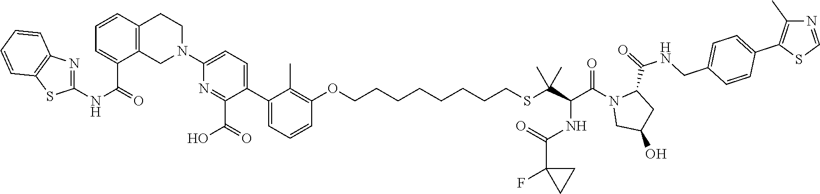 [SMILES: Cc1ncsc1-c1ccc(CNC(=O)[C@@H]2C[C@@H](O)CN2C(=O)[C@@H](NC(=O)C2(F)CC2)C(C)(C)SCCCCCCCCOc2cccc(-c3ccc(N4CCc5cccc(C(=O)Nc6nc7ccccc7s6)c5C4)nc3C(=O)O)c2C)cc1] |
| 137 | 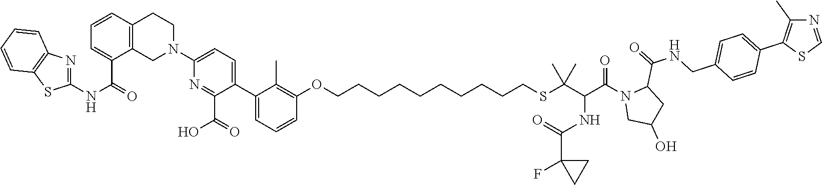 [SMILES: Cc1ncsc1-c1ccc(CNC(=O)C2CC(O)CN2C(=O)C(NC(=O)C2(F)CC2)C(C)(C)SCCCCCCCCCCOc2cccc(-c3ccc(N4CCc5cccc(C(=O)Nc6nc7ccccc7s6)c5C4)nc3C(=O)O)c2C)cc1] |
| 137a | 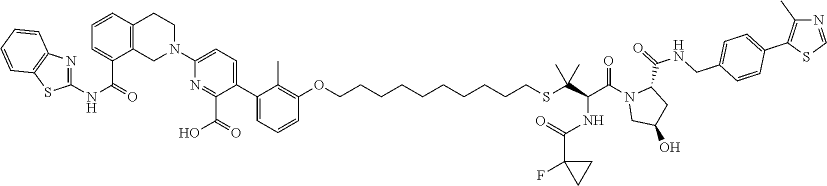 [SMILES: Cc1ncsc1-c1ccc(CNC(=O)[C@@H]2C[C@@H](O)CN2C(=O)[C@@H](NC(=O)C2(F)CC2)C(C)(C)SCCCCCCCCCCOc2cccc(-c3ccc(N4CCc5cccc(C(=O)Nc6nc7ccccc7s6)c5C4)nc3C(=O)O)c2C)cc1] |
| 138 | 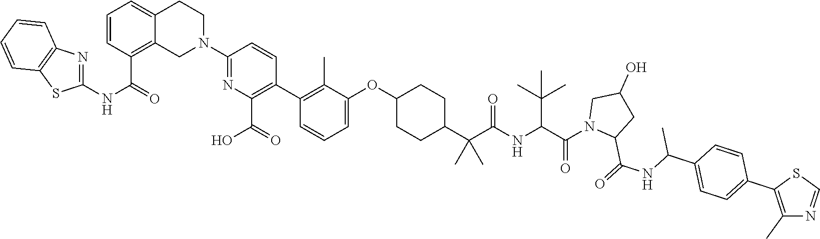 [SMILES: Cc1ncsc1-c1ccc(C(C)NC(=O)C2CC(O)CN2C(=O)C(NC(=O)C(C)(C)C2CCC(Oc3cccc(-c4ccc(N5CCc6cccc(C(=O)Nc7nc8ccccc8s7)c6C5)nc4C(=O)O)c3C)CC2)C(C)(C)C)cc1] |
| 138a | 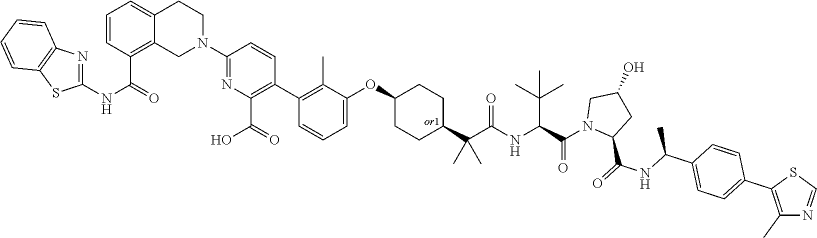 [SMILES: Cc1ncsc1-c1ccc([C@H](C)NC(=O)[C@@H]2C[C@@H](O)CN2C(=O)[C@@H](NC(=O)C(C)(C)[C@H]2CC[C@@H](Oc3cccc(-c4ccc(N5CCc6cccc(C(=O)Nc7nc8ccccc8s7)c6C5)nc4C(=O)O)c3C)CC2)C(C)(C)C)cc1] |
| 138b | 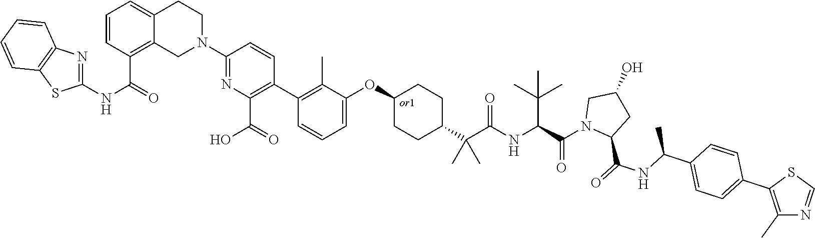 [SMILES: Cc1ncsc1-c1ccc([C@H](C)NC(=O)[C@@H]2C[C@@H](O)CN2C(=O)[C@@H](NC(=O)C(C)(C)[C@H]2CC[C@H](Oc3cccc(-c4ccc(N5CCc6cccc(C(=O)Nc7nc8ccccc8s7)c6C5)nc4C(=O)O)c3C)CC2)C(C)(C)C)cc1] |
| 139 | 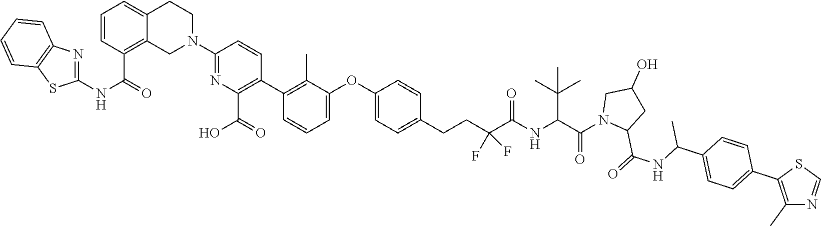 [SMILES: Cc1ncsc1-c1ccc(C(C)NC(=O)C2CC(O)CN2C(=O)C(NC(=O)C(F)(F)CCc2ccc(Oc3cccc(-c4ccc(N5CCc6cccc(C(=O)Nc7nc8ccccc8s7)c6C5)nc4C(=O)O)c3C)cc2)C(C)(C)C)cc1] |
| 139a | 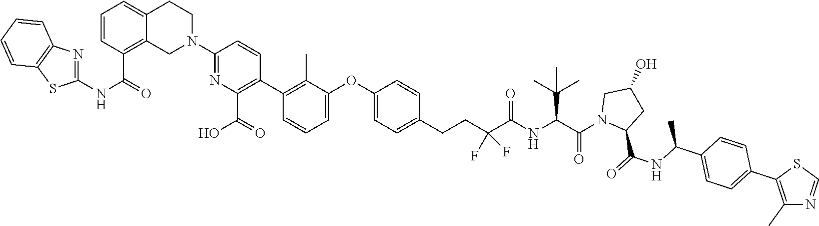 [SMILES: Cc1ncsc1-c1ccc([C@H](C)NC(=O)[C@@H]2C[C@@H](O)CN2C(=O)[C@@H](NC(=O)C(F)(F)CCc2ccc(Oc3cccc(-c4ccc(N5CCc6cccc(C(=O)Nc7nc8ccccc8s7)c6C5)nc4C(=O)O)c3C)cc2)C(C)(C)C)cc1] |
| 140 | 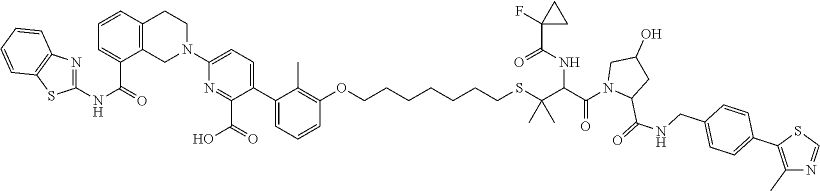 [SMILES: Cc1ncsc1-c1ccc(CNC(=O)C2CC(O)CN2C(=O)C(NC(=O)C2(F)CC2)C(C)(C)SCCCCCCCOc2cccc(-c3ccc(N4CCc5cccc(C(=O)Nc6nc7ccccc7s6)c5C4)nc3C(=O)O)c2C)cc1] |
| 140a | 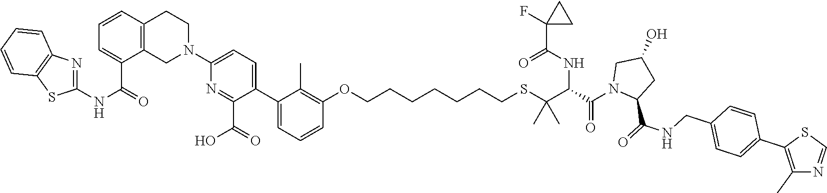 [SMILES: Cc1ncsc1-c1ccc(CNC(=O)[C@@H]2C[C@@H](O)CN2C(=O)[C@@H](NC(=O)C2(F)CC2)C(C)(C)SCCCCCCCOc2cccc(-c3ccc(N4CCc5cccc(C(=O)Nc6nc7ccccc7s6)c5C4)nc3C(=O)O)c2C)cc1] |
| 141 | 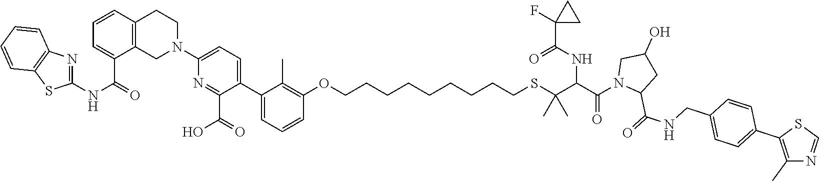 [SMILES: Cc1ncsc1-c1ccc(CNC(=O)C2CC(O)CN2C(=O)C(NC(=O)C2(F)CC2)C(C)(C)SCCCCCCCCCOc2cccc(-c3ccc(N4CCc5cccc(C(=O)Nc6nc7ccccc7s6)c5C4)nc3C(=O)O)c2C)cc1] |
| 141a | 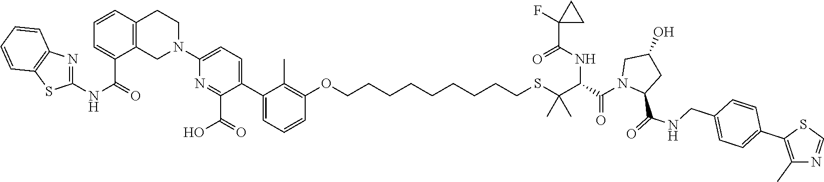 [SMILES: Cc1ncsc1-c1ccc(CNC(=O)[C@@H]2C[C@@H](O)CN2C(=O)[C@@H](NC(=O)C2(F)CC2)C(C)(C)SCCCCCCCCCOc2cccc(-c3ccc(N4CCc5cccc(C(=O)Nc6nc7ccccc7s6)c5C4)nc3C(=O)O)c2C)cc1] |
| 142 | 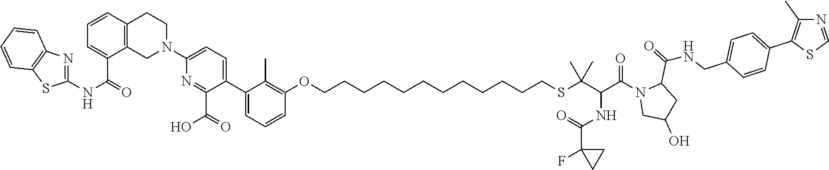 [SMILES: Cc1ncsc1-c1ccc(CNC(=O)C2CC(O)CN2C(=O)C(NC(=O)C2(F)CC2)C(C)(C)SCCCCCCCCCCCCOc2cccc(-c3ccc(N4CCc5cccc(C(=O)Nc6nc7ccccc7s6)c5C4)nc3C(=O)O)c2C)cc1] |
| 142a | 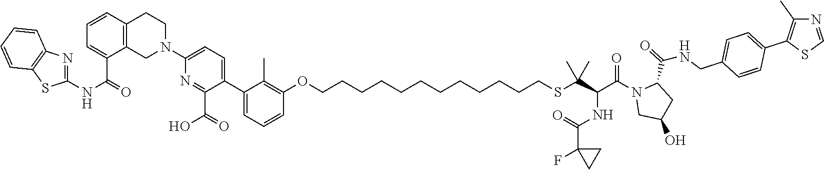 [SMILES: Cc1ncsc1-c1ccc(CNC(=O)[C@@H]2C[C@@H](O)CN2C(=O)[C@@H](NC(=O)C2(F)CC2)C(C)(C)SCCCCCCCCCCCCOc2cccc(-c3ccc(N4CCc5cccc(C(=O)Nc6nc7ccccc7s6)c5C4)nc3C(=O)O)c2C)cc1] |
| 143 | 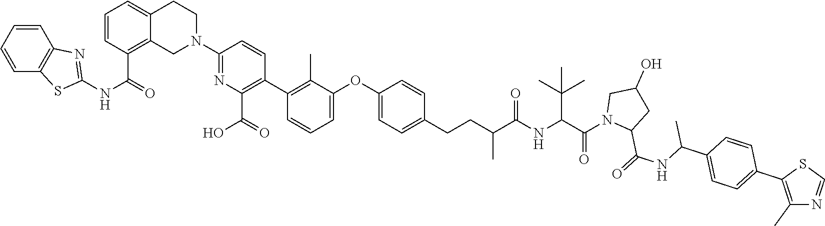 [SMILES: Cc1ncsc1-c1ccc(C(C)NC(=O)C2CC(O)CN2C(=O)C(NC(=O)C(C)CCc2ccc(Oc3cccc(-c4ccc(N5CCc6cccc(C(=O)Nc7nc8ccccc8s7)c6C5)nc4C(=O)O)c3C)cc2)C(C)(C)C)cc1] |
| 143a | 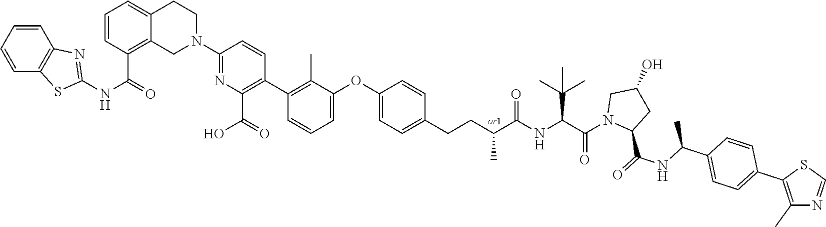 [SMILES: Cc1ncsc1-c1ccc([C@H](C)NC(=O)[C@@H]2C[C@@H](O)CN2C(=O)[C@@H](NC(=O)[C@H](C)CCc2ccc(Oc3cccc(-c4ccc(N5CCc6cccc(C(=O)Nc7nc8ccccc8s7)c6C5)nc4C(=O)O)c3C)cc2)C(C)(C)C)cc1] |
| 143b | 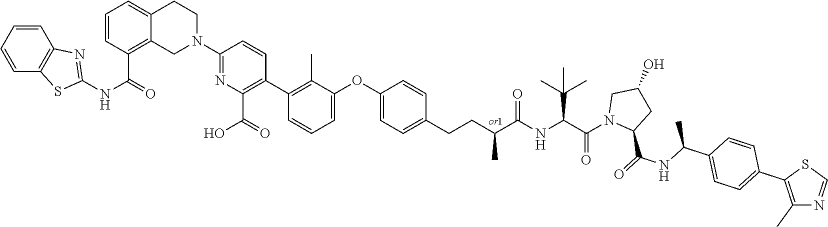 [SMILES: Cc1ncsc1-c1ccc([C@H](C)NC(=O)[C@@H]2C[C@@H](O)CN2C(=O)[C@@H](NC(=O)[C@@H](C)CCc2ccc(Oc3cccc(-c4ccc(N5CCc6cccc(C(=O)Nc7nc8ccccc8s7)c6C5)nc4C(=O)O)c3C)cc2)C(C)(C)C)cc1] |
| 144 | 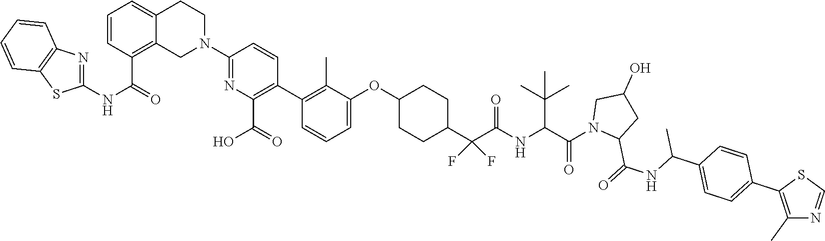 [SMILES: Cc1ncsc1-c1ccc(C(C)NC(=O)C2CC(O)CN2C(=O)C(NC(=O)C(F)(F)C2CCC(Oc3cccc(-c4ccc(N5CCc6cccc(C(=O)Nc7nc8ccccc8s7)c6C5)nc4C(=O)O)c3C)CC2)C(C)(C)C)cc1] |
| 144a | 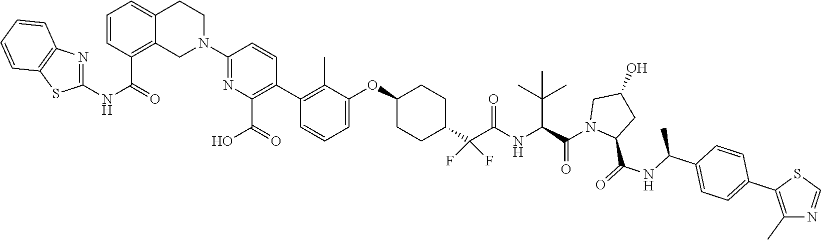 [SMILES: Cc1ncsc1-c1ccc([C@H](C)NC(=O)[C@@H]2C[C@@H](O)CN2C(=O)[C@@H](NC(=O)C(F)(F)[C@H]2CC[C@H](Oc3cccc(-c4ccc(N5CCc6cccc(C(=O)Nc7nc8ccccc8s7)c6C5)nc4C(=O)O)c3C)CC2)C(C)(C)C)cc1] |
| 144b | 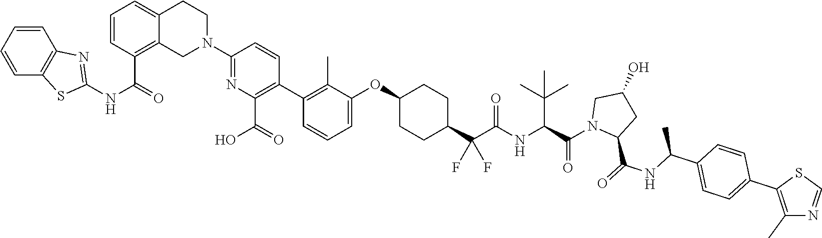 [SMILES: Cc1ncsc1-c1ccc([C@H](C)NC(=O)[C@@H]2C[C@@H](O)CN2C(=O)[C@@H](NC(=O)C(F)(F)[C@H]2CC[C@@H](Oc3cccc(-c4ccc(N5CCc6cccc(C(=O)Nc7nc8ccccc8s7)c6C5)nc4C(=O)O)c3C)CC2)C(C)(C)C)cc1] |

Note
for compounds in Table C1, when a stereogenic center is denoted with “or 1” in a structural formula, the stereogenic center has been resolved but the
configuration at the stereogenic center has not been determined. For example, the structure 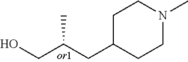
represents (R)-2-methyl-3-(1-methylpiperidin-4-yl)propan-1-ol or (S)-2-methyl-3-(1-methylpiperidin-4-yl)propan-1-ol.

In some embodiments, the compounds are selected from the group consisting of the compounds in Table C2, or pharmaceutically acceptable salts thereof.

TABLE C2

| No. | Compound Structure |
|---|---|
| 1001 | 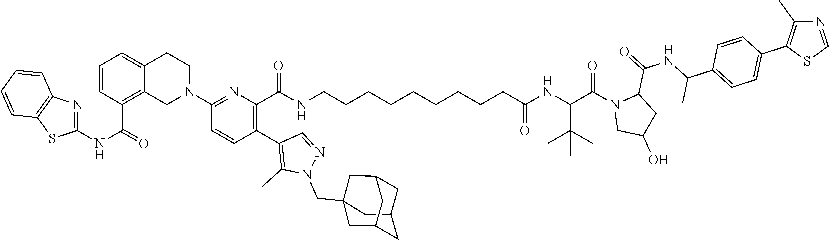 [SMILES: Cc1ncsc1-c1ccc(C(C)NC(=O)C2CC(O)CN2C(=O)C(NC(=O)CCCCCCCCCNC(=O)c2nc(N3CCc4cccc(C(=O)Nc5nc6ccccc6s5)c4C3)ccc2-c2cnn(CC34CC5CC(CC(C5)C3)C4)c2C)C(C)(C)C)cc1] |
| 10001a | 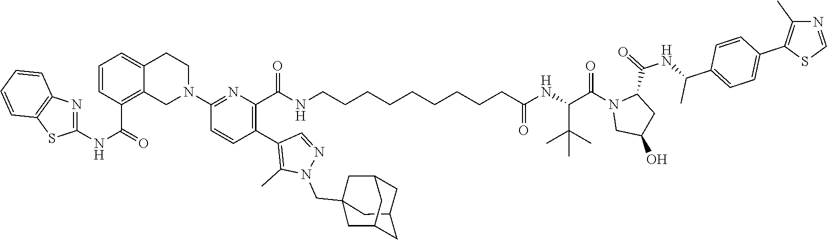 [SMILES: Cc1ncsc1-c1ccc([C@H](C)NC(=O)[C@@H]2C[C@@H](O)CN2C(=O)[C@@H](NC(=O)CCCCCCCCCNC(=O)c2nc(N3CCc4cccc(C(=O)Nc5nc6ccccc6s5)c4C3)ccc2-c2cnn(CC34CC5CC(CC(C5)C3)C4)c2C)C(C)(C)C)cc1] |
| 10002 | 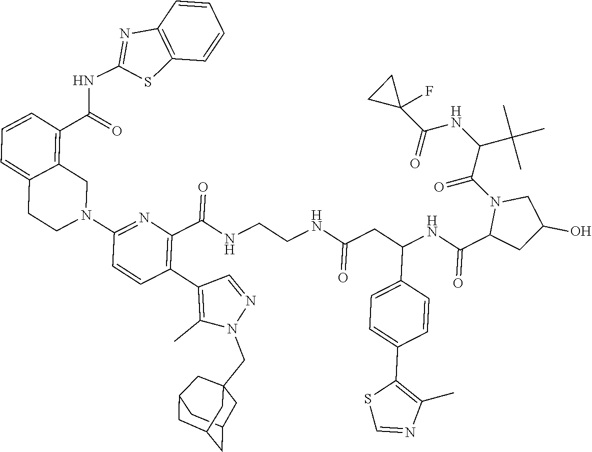 [SMILES: Cc1ncsc1-c1ccc(C(CC(=O)NCCNC(=O)c2nc(N3CCc4cccc(C(=O)Nc5nc6ccccc6s5)c4C3)ccc2-c2cnn(CC34CC5CC(CC(C5)C3)C4)c2C)NC(=O)C2CC(O)CN2C(=O)C(NC(=O)C2(F)CC2)C(C)(C)C)cc1] |
| 10002a | 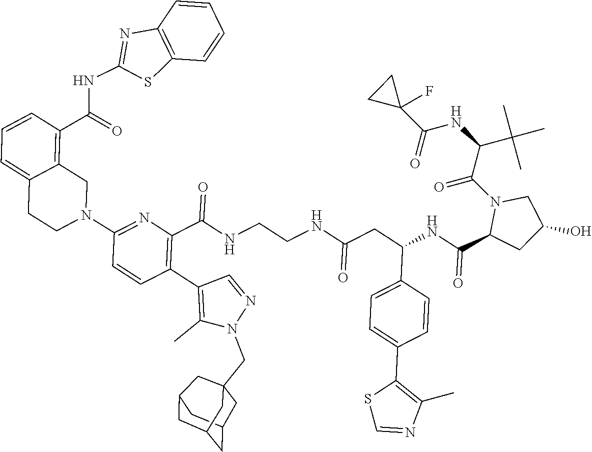 [SMILES: Cc1ncsc1-c1ccc([C@H](CC(=O)NCCNC(=O)c2nc(N3CCc4cccc(C(=O)Nc5nc6ccccc6s5)c4C3)ccc2-c2cnn(CC34CC5CC(CC(C5)C3)C4)c2C)NC(=O)[C@@H]2C[C@@H](O)CN2C(=O)[C@@H](NC(=O)C2(F)CC2)C(C)(C)C)cc1] |
| 10003 | 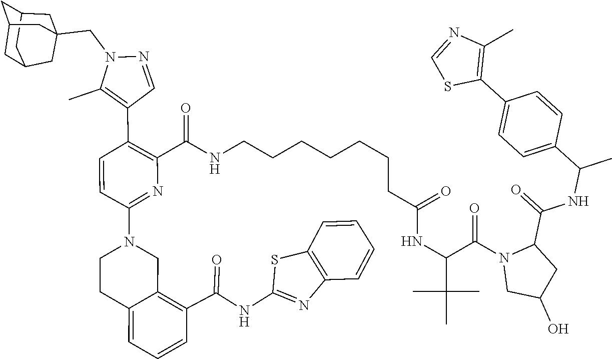 [SMILES: Cc1ncsc1-c1ccc(C(C)NC(=O)C2CC(O)CN2C(=O)C(NC(=O)CCCCCCCNC(=O)c2nc(N3CCc4cccc(C(=O)Nc5nc6ccccc6s5)c4C3)ccc2-c2cnn(CC34CC5CC(CC(C5)C3)C4)c2C)C(C)(C)C)cc1] |
| 10003a | 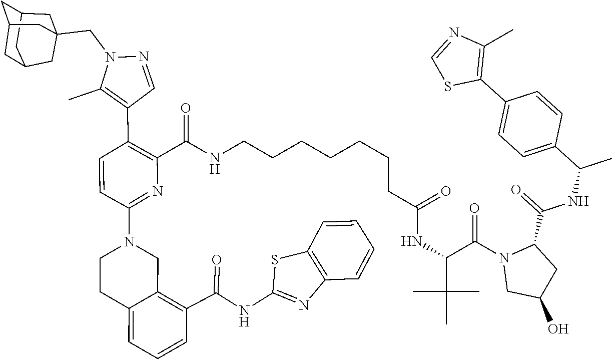 [SMILES: Cc1ncsc1-c1ccc([C@H](C)NC(=O)[C@@H]2C[C@@H](O)CN2C(=O)[C@@H](NC(=O)CCCCCCCNC(=O)c2nc(N3CCc4cccc(C(=O)Nc5nc6ccccc6s5)c4C3)ccc2-c2cnn(CC34CC5CC(CC(C5)C3)C4)c2C)C(C)(C)C)cc1] |
| 109 | 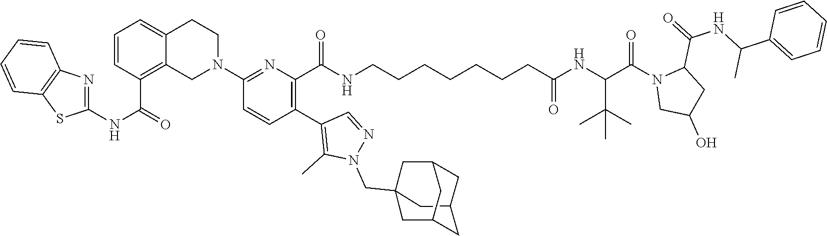 [SMILES: Cc1c(-c2ccc(N3CCc4cccc(C(=O)Nc5nc6ccccc6s5)c4C3)nc2C(=O)NCCCCCCCC(=O)NC(C(=O)N2CC(O)CC2C(=O)NC(C)c2ccccc2)C(C)(C)C)cnn1CC12CC3CC(CC(C3)C1)C2] |
| 109a | 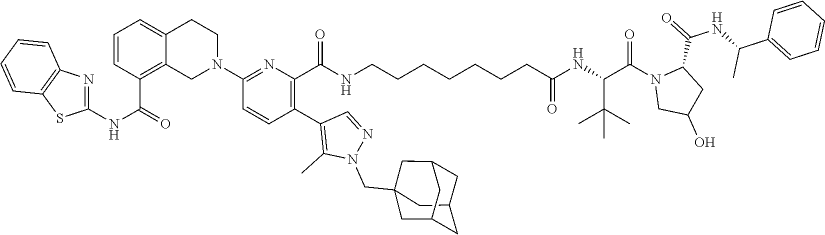 [SMILES: Cc1c(-c2ccc(N3CCc4cccc(C(=O)Nc5nc6ccccc6s5)c4C3)nc2C(=O)NCCCCCCCC(=O)N[C@H](C(=O)N2CC(O)C[C@H]2C(=O)N[C@@H](C)c2ccccc2)C(C)(C)C)cnn1CC12CC3CC(CC(C3)C1)C2] |

Exemplary compounds of Formula (I) or (II) also include those depicted in Table C1 of U.S. Provisional Application Ser. No. 63/454,486, filed Mar. 24, 2023; Table C1 of U.S. Provisional Application Ser. No. 63/449,728, filed Mar. 3, 2022; Table C1 of U.S. Provisional Application Ser. No. 63/429,834, filed Dec. 2, 2022; Table C1 of U.S. Provisional Application Ser. No. 63/398,752, filed Aug. 17, 2022; and Table C1 of U.S. Provisional Application Ser. No. 63/339,308, filed May 6, 2022; or pharmaceutically acceptable salts thereof, wherein each Table C1 is incorporated herein by reference in its entirety.

Certain examples of Formula (I) or (II) compounds were synthesized using methods involving resolution of stereoisomeric mixture(s) (e.g., SFC separation of stereoisomers). In Table C1, the resolved stereogenic centers in these compounds are labelled with the “or 1” or “or 2” enhanced stereochemical notations. In some instances, the stereoisomeric resolutions were performed during the last step of the synthesis, thereby providing the individual stereoisomers of the Formula (I) or (II) compounds. Alternatively, in some other instances, the resolutions were performed on an intermediate or starting material, wherein each of the constituent stereoisomers of the intermediate or starting material could be separately subjected to the subsequent steps of the synthesis to provide the respective Formula (I) or (II) compounds as separate stereoisomers. Methods of resolution and correlation between resolved intermediates and Formula (I) or (II) compounds are disclosed in the examples herein and in Table P1. A person of ordinary skill in the art would understand that, under either approach for stereoisomeric resolution, stereoisomers having both (R)- and (S)-configurations at a resolved stereogenic center are provided. See Table C3, wherein Table C1 compounds whose stereoisomers contain the or 1 notations are provided in non-stereogenic form, followed by the respective stereoisomers having the (R)- and (S)-configurations.

TABLE C3

| No. | Compound Structure |
|---|---|
| 129 | 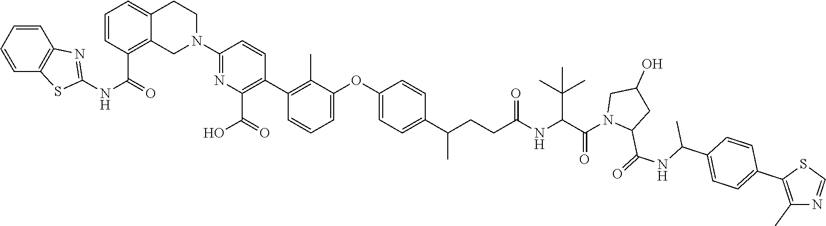 [SMILES: Cc1ncsc1-c1ccc(C(C)NC(=O)C2CC(O)CN2C(=O)C(NC(=O)CCC(C)c2ccc(Oc3cccc(-c4ccc(N5CCc6cccc(C(=O)Nc7nc8ccccc8s7)c6C5)nc4C(=O)O)c3C)cc2)C(C)(C)C)cc1] |
| | 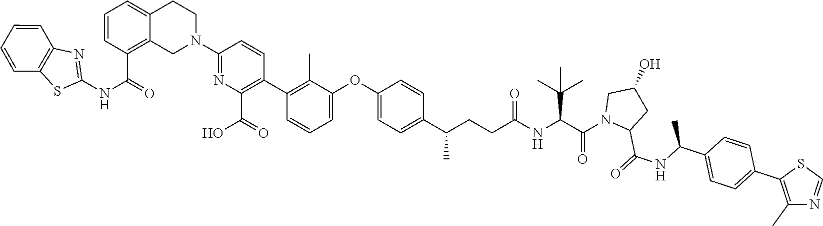 [SMILES: Cc1ncsc1-c1ccc([C@H](C)NC(=O)C2C[C@@H](O)CN2C(=O)[C@@H](NC(=O)CC[C@H](C)c2ccc(Oc3cccc(-c4ccc(N5CCc6cccc(C(=O)Nc7nc8ccccc8s7)c6C5)nc4C(=O)O)c3C)cc2)C(C)(C)C)cc1] |
| | 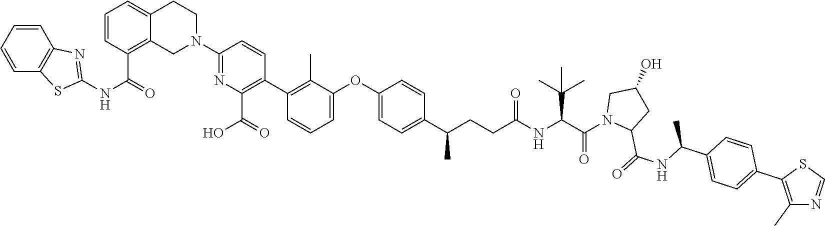 [SMILES: Cc1ncsc1-c1ccc([C@H](C)NC(=O)C2C[C@@H](O)CN2C(=O)[C@@H](NC(=O)CC[C@@H](C)c2ccc(Oc3cccc(-c4ccc(N5CCc6cccc(C(=O)Nc7nc8ccccc8s7)c6C5)nc4C(=O)O)c3C)cc2)C(C)(C)C)cc1] |
| 131 | 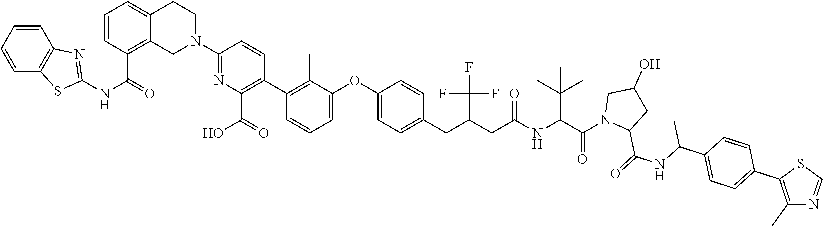 [SMILES: Cc1ncsc1-c1ccc(C(C)NC(=O)C2CC(O)CN2C(=O)C(NC(=O)CC(Cc2ccc(Oc3cccc(-c4ccc(N5CCc6cccc(C(=O)Nc7nc8ccccc8s7)c6C5)nc4C(=O)O)c3C)cc2)C(F)(F)F)C(C)(C)C)cc1] |
| | 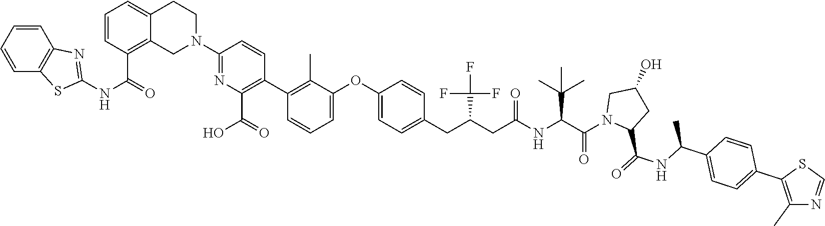 |
| | 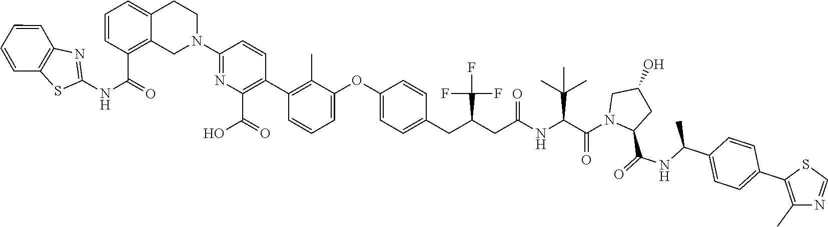 [SMILES: Cc1ncsc1-c1ccc([C@H](C)NC(=O)[C@@H]2C[C@@H](O)CN2C(=O)[C@@H](NC(=O)C[C@@H](Cc2ccc(Oc3cccc(-c4ccc(N5CCc6cccc(C(=O)Nc7nc8ccccc8s7)c6C5)nc4C(=O)O)c3C)cc2)C(F)(F)F)C(C)(C)C)cc1] |
| 134 | 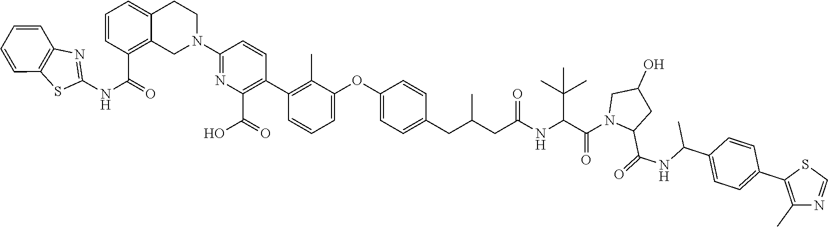 [SMILES: Cc1ncsc1-c1ccc(C(C)NC(=O)C2CC(O)CN2C(=O)C(NC(=O)CC(C)Cc2ccc(Oc3cccc(-c4ccc(N5CCc6cccc(C(=O)Nc7nc8ccccc8s7)c6C5)nc4C(=O)O)c3C)cc2)C(C)(C)C)cc1] |
| | 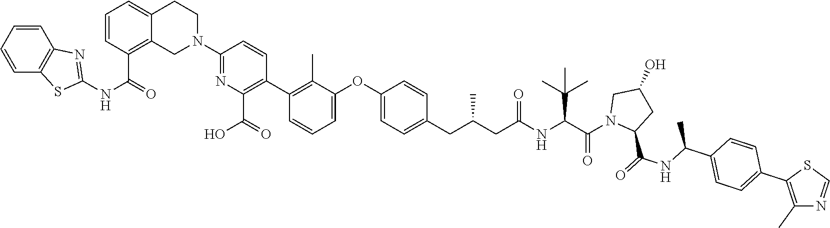 [SMILES: Cc1ncsc1-c1ccc([C@H](C)NC(=O)[C@@H]2C[C@@H](O)CN2C(=O)[C@@H](NC(=O)C[C@@H](C)Cc2ccc(Oc3cccc(-c4ccc(N5CCc6cccc(C(=O)Nc7nc8ccccc8s7)c6C5)nc4C(=O)O)c3C)cc2)C(C)(C)C)cc1] |
| | 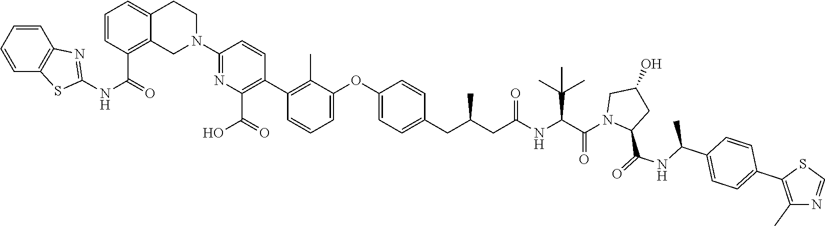 [SMILES: Cc1ncsc1-c1ccc([C@H](C)NC(=O)[C@@H]2C[C@@H](O)CN2C(=O)[C@@H](NC(=O)C[C@H](C)Cc2ccc(Oc3cccc(-c4ccc(N5CCc6cccc(C(=O)Nc7nc8ccccc8s7)c6C5)nc4C(=O)O)c3C)cc2)C(C)(C)C)cc1] |
| 135 | 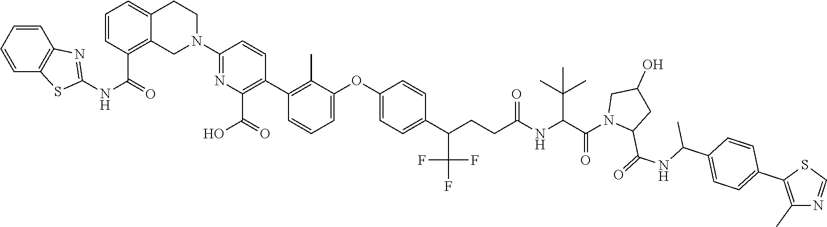 [SMILES: Cc1ncsc1-c1ccc(C(C)NC(=O)C2CC(O)CN2C(=O)C(NC(=O)CCC(c2ccc(Oc3cccc(-c4ccc(N5CCc6cccc(C(=O)Nc7nc8ccccc8s7)c6C5)nc4C(=O)O)c3C)cc2)C(F)(F)F)C(C)(C)C)cc1] |
| | 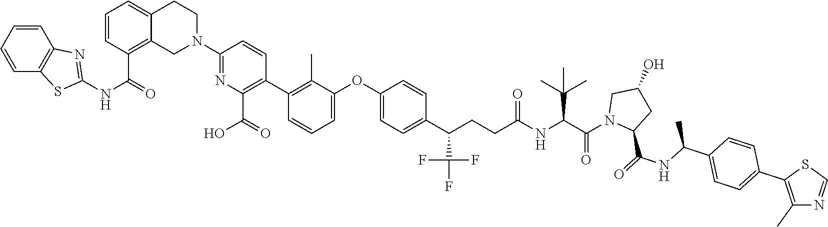 [SMILES: Cc1ncsc1-c1ccc([C@H](C)NC(=O)[C@@H]2C[C@@H](O)CN2C(=O)[C@@H](NC(=O)CC[C@@H](c2ccc(Oc3cccc(-c4ccc(N5CCc6cccc(C(=O)Nc7nc8ccccc8s7)c6C5)nc4C(=O)O)c3C)cc2)C(F)(F)F)C(C)(C)C)cc1] |
| | 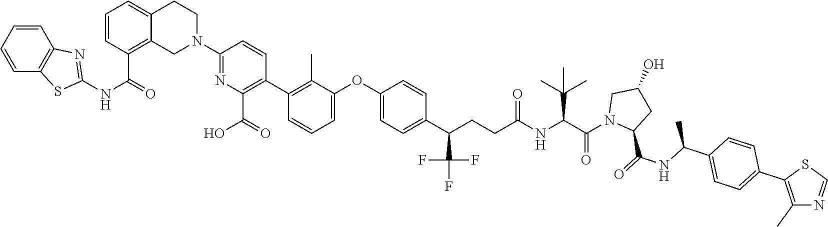 [SMILES: Cc1ncsc1-c1ccc([C@H](C)NC(=O)[C@@H]2C[C@@H](O)CN2C(=O)[C@@H](NC(=O)CC[C@H](c2ccc(Oc3cccc(-c4ccc(N5CCc6cccc(C(=O)Nc7nc8ccccc8s7)c6C5)nc4C(=O)O)c3C)cc2)C(F)(F)F)C(C)(C)C)cc1] |
| 138 | 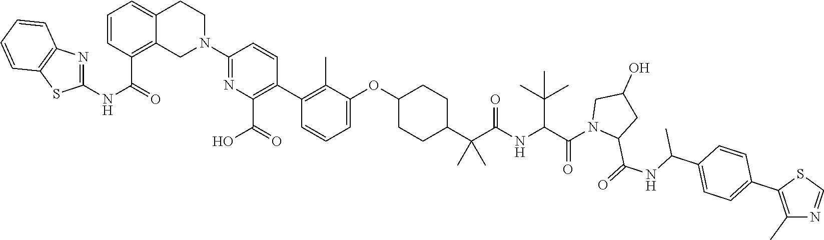 [SMILES: Cc1ncsc1-c1ccc(C(C)NC(=O)C2CC(O)CN2C(=O)C(NC(=O)C(C)(C)C2CCC(Oc3cccc(-c4ccc(N5CCc6cccc(C(=O)Nc7nc8ccccc8s7)c6C5)nc4C(=O)O)c3C)CC2)C(C)(C)C)cc1] |
| | 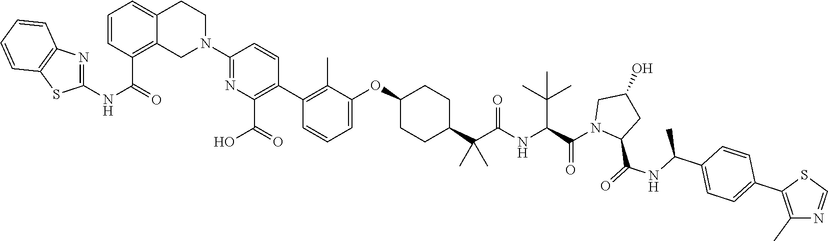 [SMILES: Cc1ncsc1-c1ccc([C@H](C)NC(=O)[C@@H]2C[C@@H](O)CN2C(=O)[C@@H](NC(=O)C(C)(C)[C@H]2CC[C@@H](Oc3cccc(-c4ccc(N5CCc6cccc(C(=O)Nc7nc8ccccc8s7)c6C5)nc4C(=O)O)c3C)CC2)C(C)(C)C)cc1] |
| | 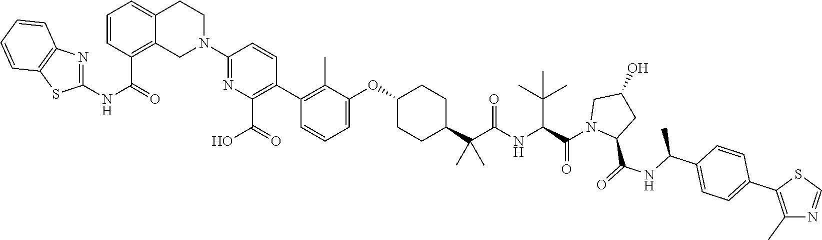 [SMILES: Cc1ncsc1-c1ccc([C@H](C)NC(=O)[C@@H]2C[C@@H](O)CN2C(=O)[C@@H](NC(=O)C(C)(C)[C@H]2CC[C@H](Oc3cccc(-c4ccc(N5CCc6cccc(C(=O)Nc7nc8ccccc8s7)c6C5)nc4C(=O)O)c3C)CC2)C(C)(C)C)cc1] |
| 143 | 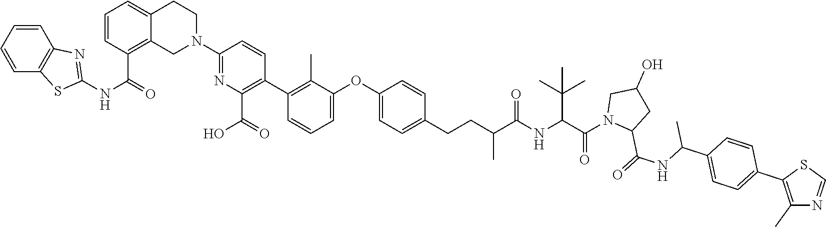 [SMILES: Cc1ncsc1-c1ccc(C(C)NC(=O)C2CC(O)CN2C(=O)C(NC(=O)C(C)CCc2ccc(Oc3cccc(-c4ccc(N5CCc6cccc(C(=O)Nc7nc8ccccc8s7)c6C5)nc4C(=O)O)c3C)cc2)C(C)(C)C)cc1] |
| | 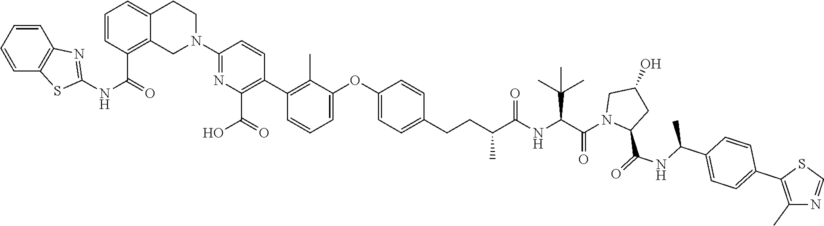 [SMILES: Cc1ncsc1-c1ccc([C@H](C)NC(=O)[C@@H]2C[C@@H](O)CN2C(=O)[C@@H](NC(=O)[C@H](C)CCc2ccc(Oc3cccc(-c4ccc(N5CCc6cccc(C(=O)Nc7nc8ccccc8s7)c6C5)nc4C(=O)O)c3C)cc2)C(C)(C)C)cc1] |
| | 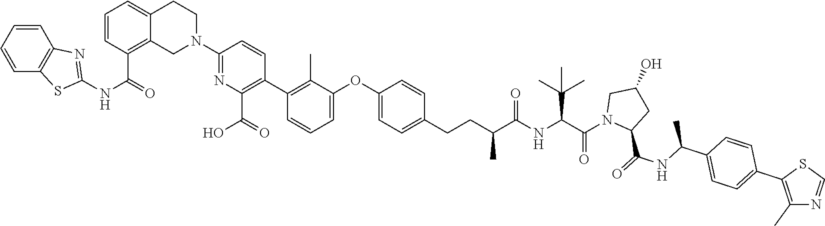 [SMILES: Cc1ncsc1-c1ccc([C@H](C)NC(=O)[C@@H]2C[C@@H](O)CN2C(=O)[C@@H](NC(=O)[C@@H](C)CCc2ccc(Oc3cccc(-c4ccc(N5CCc6cccc(C(=O)Nc7nc8ccccc8s7)c6C5)nc4C(=O)O)c3C)cc2)C(C)(C)C)cc1] |

In some embodiments, the compounds of Formula (I) or (II), or pharmaceutically acceptable salts thereof, reduce cell viability in a cell line expressing a BCL-X_{L} protein with an EC₅₀ of less than 1 μM (e.g., less than 750 nM, less than 500 nM, or less than 200 nM). In some embodiments, the compounds reduce cell viability in a cell line expressing the BCL-X_{L} protein with an EC₅₀ of less than 200 nM (e.g., less than 150 nM, less than 200 nM, less than 100 nM, less than 10 nM, less than 1 nM). For example, the compounds can reduce cell viability in a cell line expressing the BCL-X_{L} protein with an EC₅₀ of between 0.1 nM to 100 nM, between 0.1 nM to 50 nM, between 1 nM to 50 nM, between 1 nM to 20 nM, or between 0.1 nM to 1 nM.

In some embodiments, the compounds of Formula (I) or (II), or pharmaceutically acceptable salts thereof, induce degradation of a BCL-X_{L} protein in a cell line expressing the BCL-X_{L} protein with a DC₅₀ of less than 1 μM (e.g., less than 750 nM, less than 500 nM, or less than 200 nM). In some embodiments, the compounds of Formula (I) or (II), or a pharmaceutically acceptable salt thereof, induce degradation of a BCL-X_{L} protein in a cell line expressing the BCL-X_{L} protein with a DC₅₀ of less than 200 nM (e.g., less than 150 nM, less than 200 nM, less than 100 nM, less than 10 nM, less than 1 nM). For example, the compounds can induce degradation of a BCL-X_{L} protein in a cell line expressing the BCL-X_{L} protein with a DC₅₀ of between 0.1 nM to 100 nM, between 0.1 nM to 50 nM, between 1 nM to 50 nM, between 1 nM to 20 nM, or between 0.1 nM to 1 nM.

In some embodiments, the compounds of Formula (I) or (II), or pharmaceutically acceptable salts thereof, induce degradation of a BCL-X_{L} protein in a cell line expressing the BCL-X_{L} protein with a Yₘᵢₙ of less than 70% (e.g., less than 50%, less than 30%, less than 20%, or less than 10%). In some embodiments, the compounds of Formula (I) or (II), or a pharmaceutically acceptable salt thereof, induce degradation of a BCL-X_{L} protein in a cell line expressing the BCL-X_{L} protein with a Yₘᵢₙ of less than 50% (e.g., less than 40%, less than 30%, less than 20%, less than 10%, or less than 5%). In some embodiments, the compounds of Formula (I) or (II), or a pharmaceutically acceptable salt thereof, induce degradation of a BCL-X_{L} protein in a cell line expressing the BCL-X_{L} protein with a Yₘᵢₙ of less than 30% (e.g., less than 25%, less than 20%, less than 15%, less than 10%, or less than 5%). For example, the compounds can induce degradation of a BCL-X_{L} protein in a cell line expressing the BCL-X_{L} protein with a Yₘᵢₙ of about 1% to about 70% (e.g., about 5% to about 50% or about 10% to about 30%).

Also provided herein is a BCL-X_{L} protein non-covalently bound with a compound of Formula (I) or (II), or a pharmaceutically acceptable salt thereof.

Also provided herein is a ternary complex comprising a BCL-X_{L} protein, a compound of Formula (I) or (II), or a pharmaceutically acceptable salt thereof, and a VHL protein, or a portion thereof.

### Chemical Definitions

The term “halo” refers to fluoro (F), chloro (Cl), bromo (Br), or iodo (I).

The term “oxo” refers to a divalent doubly bonded oxygen atom (i.e., “═O”). As used herein, oxo groups are attached to carbon atoms to form carbonyls.

The term “alkyl” refers to a saturated acyclic hydrocarbon radical that may be a straight chain or branched chain, containing the indicated number of carbon atoms. For example, C₁₋₁₀ indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “haloalkyl” refers to an alkyl, in which one or more hydrogen atoms is/are replaced with an independently selected halo (e.g., —CF₃, —CHF₂, or —CH₂F).

The term “alkoxy” refers to an —O-alkyl radical (e.g., —OCH₃).

The term “alkylene” refers to a divalent alkyl (e.g., —CH₂—). Similarly, terms such as “cycloalkylene” and “heterocyclylene” refer to divalent cycloalkyl and heterocyclyl respectively. For avoidance of doubt, in “cycloalkylene” and “heterocyclylene”, the two radicals can be on the same ring carbon atom (e.g., a geminal diradical such as

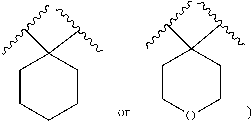 [SMILES: *C1(*)CCCCC1 ; *C1(*)CCOCC1]

or on different ring atoms (e.g., ring carbon and/or nitrogen atoms (e.g., vicinal ring carbon and/or nitrogen atoms))

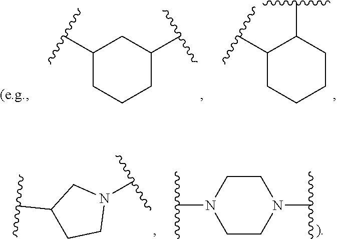 [SMILES: *C1CCCC(*)C1 ; *C1CCCCC1* ; *C1CCN(*)C1 ; *N1CCN(*)CC1]

The term “alkenyl” refers to an acyclic hydrocarbon chain that may be a straight chain or branched chain having one or more carbon-carbon double bonds. The alkenyl moiety contains the indicated number of carbon atoms. For example, C₂₋₆ indicates that the group may have from 2 to 6 (inclusive) carbon atoms in it. Alkenyl groups can either be unsubstituted or substituted with one or more substituents.

The term “alkynyl” refers to an acyclic hydrocarbon chain that may be a straight chain or branched chain having one or more carbon-carbon triple bonds. The alkynyl moiety contains the indicated number of carbon atoms. For example, C₂₋₆ indicates that the group may have from 2 to 6 (inclusive) carbon atoms in it. Alkynyl groups can either be unsubstituted or substituted with one or more substituents.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, and the like.

The term “cycloalkyl” as used herein refers to mono-, bi-, tri-, or polycyclic (e.g., fused, bridged, or spirocyclic bi-, tri-, or polycyclic) saturated or partially unsaturated hydrocarbon groups having, e.g., 3 to 20 ring carbons, preferably 3 to 15 ring carbons, and more preferably 3 to 12 ring carbons or 3-10 ring carbons or 3-6 ring carbons, wherein the cycloalkyl group may be optionally substituted. The term “saturated” as used in this context means only single bonds present between constituent carbon atoms. Examples of saturated cycloalkyl groups include, without limitation, cyclopropyl, cyclobutyl, cyclopentyl, cyclohexyl, cycloheptyl, and cyclooctyl. Partially unsaturated cycloalkyl may have any degree of unsaturation provided that one or more double bonds is present in the cycloalkyl, none of the rings in the ring system are aromatic, and the partially unsaturated cycloalkyl group is not fully saturated overall. Examples of partially unsaturated cycloalkyl include, without limitation, cyclopentenyl, cyclohexenyl, cycloheptenyl, and cyclooctenyl. Non-limiting examples of fused/bridged cycloalkyl includes: bicyclo[1.1.0]butyl, bicyclo[2.1.0]pentyl, bicyclo[1.1.1]pentyl, bicyclo[3.1.0]hexyl, bicyclo[2.1.1]hexyl, bicyclo[3.2.0]heptyl, bicyclo[4.1.0]heptyl, bicyclo[2.2.1]heptyl, bicyclo[3.1.1]heptyl, bicyclo[4.2.0]octyl, bicyclo[3.2.1]octyl, bicyclo[2.2.2]octyl, and the like. Non-limiting examples of spirocyclic cycloalkyls include spiro[2.2]pentyl, spiro[2.5]octyl, spiro[3.5]nonyl, spiro[3.5]nonyl, spiro[3.5]nonyl, spiro[4.4]nonyl, spiro[2.6]nonyl, spiro[4.5]decyl, spiro[3.6]decyl, spiro[5.5]undecyl, and the like.

The term “heteroaryl”, as used herein, means a mono-, bi-, tri- or polycyclic group having 5 to 20 ring atoms, alternatively 5, 6, 9, 10, or 15 ring atoms; wherein at least one ring in the system contains one or more heteroatoms independently selected from the group consisting of N, O, S (inclusive of oxidized forms such as:

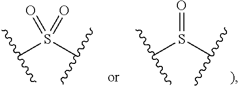

and P (inclusive of oxidized forms such as:

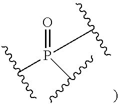 [SMILES: *P(*)(*)=O]

and at least one ring in the system is aromatic (but does not have to be a ring which contains a heteroatom, e.g. tetrahydroisoquinolinyl, e.g., tetrahydroquinolinyl). Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromanyl, 2,3-dihydrobenzo[b][1,4]dioxinyl, benzo[d][1,3]dioxolyl, 2,3-dihydrobenzofuranyl, tetrahydroquinolinyl, 2,3-dihydrobenzo[b][1,4]oxathiinyl, isoindolinyl, and others. In some embodiments, the heteroaryl is selected from thienyl, pyridinyl, furyl, pyrazolyl, imidazolyl, isoindolinyl, pyranyl, pyrazinyl, and pyrimidinyl. For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone

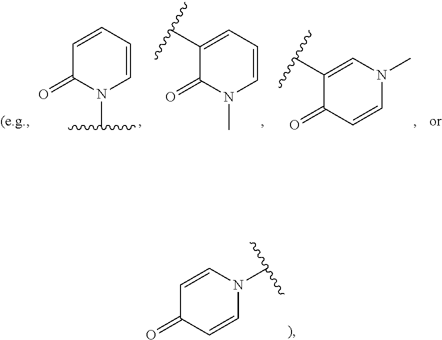

pyrimidone

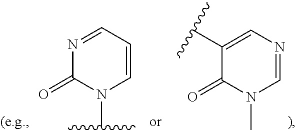

pyridazinone

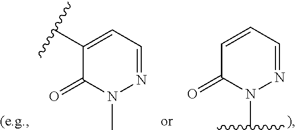

pyrazinone

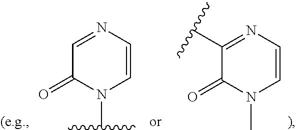

and imidazolone

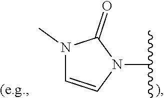 [SMILES: *n1ccn(C)c1=O]

wherein each ring nitrogen adjacent to a carbonyl is tertiary (i.e., the oxo group (i.e., “═O”) herein is a constituent part of the heteroaryl ring).

The term “heterocyclyl” refers to a mono-, bi-, tri-, or polycyclic (e.g., fused, bridged, or spirocyclic bi-, tri-, or polycyclic) saturated or partially unsaturated ring system with 3-15 ring atoms (e.g., 5-8 membered monocyclic, 8-12 membered bicyclic, or 11-15 membered tricyclic ring system) having 1-3 heteroatoms if monocyclic, 1-6 heteroatoms if bicyclic, or 1-9 heteroatoms if tricyclic or polycyclic, said heteroatoms selected from O, N, S (inclusive of oxidized forms such as:

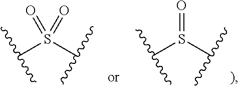

P (inclusive of oxidized forms such as:

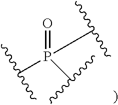 [SMILES: *P(*)(*)=O]

(e.g., carbon atoms and 1-3, 1-6, or 1-9 heteroatoms of N, O, S, or P if monocyclic, bicyclic, or tricyclic, respectively), wherein 0, 1, 2 or 3 atoms of each ring may be substituted by a substituent. The term “saturated” as used in this context means only single bonds present between constituent ring atoms and other available valences occupied by hydrogen and/or other substituents as defined herein. Examples of saturated heterocyclyl groups include piperazinyl, pyrrolidinyl, dioxanyl, morpholinyl, tetrahydrofuranyl, and the like. Partially unsaturated heterocyclyl groups may have any degree of unsaturation provided that one or more double bonds is present in the heterocyclyl, none of the rings in the ring system are aromatic, and the partially unsaturated heterocyclyl group is not fully saturated overall. Examples of partially unsaturated heterocyclyl groups include, without limitation, tetrahydropyridyl, dihydropyrazinyl, dihydropyridyl, dihydropyrrolyl, dihydrofuranyl, dihydrothiophenyl. Non-limiting examples of fused/bridged heteorocyclyl includes: 2-azabicyclo[1.1.0]butyl, 2-azabicyclo[2.1.0]pentyl, 2-azabicyclo[1.1.1]pentyl, 3-azabicyclo[3.1.0]hexyl, 5-azabicyclo[2.1.1]hexyl, 3-azabicyclo[3.2.0]heptyl, octahydrocyclopenta[c]pyrrole, 3-azabicyclo[4.1.0]heptyl, 7-azabicyclo[2.2.1]heptyl, 6-azabicyclo[3.1.1]heptyl, 7-azabicyclo[4.2.0]octyl, 2-azabicyclo[2.2.2]octyl, 3-azabicyclo[3.2.1]octyl, 2-oxabicyclo[1.1.0]butyl, 2-oxabicyclo[2.1.0]pentyl, 2-oxabicyclo[1.1.1]pentyl, 3-oxabicyclo[3.1.0]hexyl, 5-oxabicyclo[2.1.1]hexyl, 3-oxabicyclo[3.2.0]heptyl, 3-oxabicyclo[4.1.0]heptyl, 7-oxabicyclo[2.2.1]heptyl, 6-oxabicyclo[3.1.1]heptyl, 7-oxabicyclo[4.2.0]octyl, 2-oxabicyclo[2.2.2]octyl, 3-oxabicyclo[3.2.1]octyl, and the like. Non-limiting examples of spirocyclic heterocyclyls include 2-azaspiro[2.2]pentyl, 4-azaspiro[2.5]octyl, 1-azaspiro[3.5]nonyl, 2-azaspiro[3.5]nonyl, 7-azaspiro[3.5]nonyl, 2-azaspiro[4.4]nonyl, 6-azaspiro[2.6]nonyl, 1,7-diazaspiro[4.5]decyl, 7-azaspiro[4.5]decyl 2,5-diazaspiro[3.6]decyl, 3-azaspiro[5.5]undecyl, 2-oxaspiro[2.2]pentyl, 4-oxaspiro[2.5]octyl, 1-oxaspiro[3.5]nonyl, 2-oxaspiro[3.5]nonyl, 7-oxaspiro[3.5]nonyl, 2-oxaspiro[4.4]nonyl, 6-oxaspiro[2.6]nonyl, 1,7-dioxaspiro[4.5]decyl, 2,5-dioxaspiro[3.6]decyl, 1-oxaspiro[5.5]undecyl, 3-oxaspiro[5.5]undecyl, 3-oxa-9-azaspiro[5.5]undecyl and the like.

As used herein, when a ring is described as being “partially unsaturated”, it means said ring has one or more additional degrees of unsaturation (in addition to the degree of unsaturation attributed to the ring itself, e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, heterocycloalkenyl, cycloalkenyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

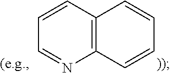

(ii) a single ring atom (spiro-fused ring systems)

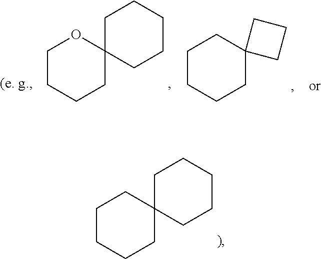

or (iii) a contiguous array of ring atoms (bridged ring systems having all bridge lengths >0)

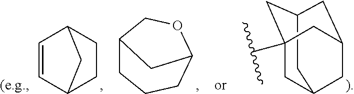

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

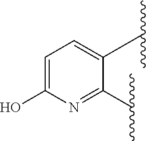 [SMILES: *c1ccc(O)nc1*]

encompasses the tautomeric form containing the moiety:

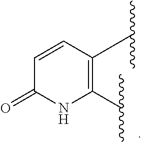 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The compounds provided herein may encompass various stereochemical forms. The compounds also encompass diastereomers as well as optical isomers, e.g., mixtures of enantiomers including racemic mixtures, as well as individual enantiomers and diastereomers, which arise as a consequence of structural asymmetry in certain compounds. Unless otherwise indicated, when a disclosed compound is named or depicted by a structure without specifying the stereochemistry and has one or more chiral centers, it is understood to represent all possible stereoisomers of the compound.

### Methods of Treatment

### Indications

Provided herein are methods for inducing degradation of a BCL-X_{L} protein. For example, provided herein are compounds capable of inducing degradation of a BCL-X_{L} protein useful for treating or preventing cancers. See, e.g., Guo, et al. Aging (Albany NY) 13.15 (2021): 19750; Park, et al. Proceedings of the National Academy of Sciences 112.40 (2015): 12492-12497; Zhang, et al. Molecular Cancer 14.1 (2015): 1-9; Beroukhim, et al. Nature 463.7283 (2010): 899-905. Additional methods of evaluating the binding of a compound to BCL-X_{L} protein or the inhibition of a BCL-X_{L} protein are described in, for example, U.S. Patent Publication Nos. 2007/027135; 2010/305122, and 2013/096120.

It will be understood that the effect of protein degradation typically increases over time, though the appearance of degradation (e.g., as expressed by the percentage degradation compared to a control, or the parameters Yₘᵢₙ, DC₅₀, and/or Dₘₐₓ) is affected by the resynthesis rate of the protein. It is common in the art to examine degradation after a specified period of time, such as 6 hours, 12 hours, 18 hours, 1 day, 2 days, 3 days, or more. For example, degradation can be expressed as the percent degradation after 24 hours.

Exemplary assays for validating the degradation-inducing mechanism of a compound as provided herein are known in the art and are described, for example, in International Publication No. WO 2019/144117 and Wu, et al. Nature Structural & Molecular Biology 27.7 (2020): 605-614.

Degradation assays can be used to quantify both on- and off-target degradation-inducing effects of compounds, such as those provided herein. Exemplary assays include, quantitative immunoblotting, other immunoassays (e.g., MesoScale Discovery (MSD) immunoassays), homogenous time resolved florescence (HTRF), and HiBiT. In some embodiments, cells can be contacted with a compound of Formula (I) or (II), or a pharmaceutically acceptable salt thereof, incubated, and then the lysate can be prepared for gel electrophoresis (e.g., SDS-PAGE), followed by immunoblotting and quantification compared to a control (e.g., a DMSO-treated control). As another example, a cell line can be engineered to express a HiBiT-tagged BCL-X_{L} protein, and the amount of fluorescence observed when the complementary LgBiT peptide is added can be compared between cells treated with a compound of Formula (I) or (II), or a pharmaceutically acceptable salt thereof, and a control (e.g., a DMSO-treated control). See, for instance, Example B1 and Example B2. See also, e.g., International Publication Nos. WO 2020/163823 and WO 2019/144117. In some embodiments, off-target degradation inducing effects can be assessed for the protein cysteine dioxygenase 1 (CDO1).

See also the assays described in International Publication Nos. WO 2023/044046; WO 2022/169780; WO 2021/222114; WO 2021/146536; WO 2021/078301; WO 2021/007307; WO 2020/163823; WO 2019/144117; WO 2017/184995, and Khan, et al. Nature Medicine 25.12 (2019): 1938-1947; Balachander, et al. Clinical Cancer Research 26.24 (2020): 6535-6549.

Binding affinity of a compound of Formula (I) or (II), or a pharmaceutically acceptable salt thereof, as provided herein to BCL-X_{L} can be determined by, for example, a binding IC₅₀ or Kᵢ value (e.g., using a competition assay), or by a K_{D} value (e.g., using a biophysical assay). A compound with a lower binding IC₅₀ value, as determined under substantially similar conditions, is a more potent binder relative to a compound with a higher binding IC₅₀ value. A compound with a lower binding Kᵢ value, as determined under substantially similar conditions, is a more potent binder relative to a compound with a higher binding Kᵢ value. Similarly, a compound with a lower K_{D} value, as determined under substantially similar conditions, is a more potent binder relative to a compound with a higher K_{D} value. For example, a binding IC₅₀ value can be determined in a fluorescence polarization assay using a fluorescently labeled BH3-only peptide (e.g., BAD or BAX) as the competitor. As another example, a binding Kᵢ value can be determined using a time resolved-fluorescence resonance energy transfer (TR-FRET) assay using a fluorescently labeled BH3-only peptide (e.g., BAK) and a fluorescently labeled antibody that binds to BCL-X_{L}, where the fluorophores are a FRET pair, and using a compound of Formula (I) or (II), or a pharmaceutically acceptable salt thereof, as provided herein as a competitor for the BH3-only peptide. See, e.g., U.S. Patent Publication Nos. 2007/0027135, 2010/305122, and 2013/096120.

The ability of a compound of Formula (I) or (II), or a pharmaceutically acceptable salt thereof, as provided herein to inhibit BCL-X_{L} can be determined using an IC₅₀ value. A compound with a lower IC₅₀ value, as determined under substantially similar conditions, is a more potent inhibitor relative to a compound with a higher IC₅₀ value. One way that inhibition of BCL-X_{L} can be measured is measuring the interruption of the formation of a complex of BCL-X_{L} with a BH3-only peptide (e.g., BIM). For example, an electrochemiluminescence-based sandwich ELISA assay (e.g., a Meso Scale Discovery (MSD)-ELISA assay) can be used. See, for example, Phillips, D. C., et al. Blood Cancer Journal 5.11 (2015): e368-e368 and Xiao, Yu, et al. Molecular Cancer Therapeutics 14.8 (2015): 1837-1847. In such assays, cells expressing BCL-X_{L} can be incubated with a compound of Formula (I) or (II) for a period of time, lysed, and then evaluated in the assay. A tagged anti-BCL-X_{L} antibody (e.g., a biotin-tagged anti-BCL-X_{L} antibody) can be immobilized on an assay plate (e.g., a streptavidin assay plate), and then the lysate can be applied to pull down BCL-X_{L}. An anti-BIM antibody (e.g., a rabbit anti-BIM antibody) can be introduced, followed by addition of a detection antibody (e.g., a sulfo-tagged goat anti-rabbit antibody) and measurement of the detection antibody. As another example, interruption of the formation of a complex of BCL-X_{L} with a BH3-only peptide (e.g., BIM) can be measured using a mammalian two-hybrid assay. In such assays, a plasmid encoding the ‘bait’ and ‘prey’ fusion proteins (e.g., the DNA binding domain of GAL4 fused to BCL-X_{L} and the transcriptional activation domain of VP16 fused to BIM) can be introduced to cells (e.g., HeLa cells) stably expressing a GAL4-luciferase reporter. A compound of Formula (I) or (II) can be added to the cells in culture, incubated, and the luciferase activity can be measured. See, for example, Souers, Andrew J., et al. Nature Medicine 19.2 (2013): 202-208.

Potency of degradation by a compound of Formula (I) or (II), or a pharmaceutically acceptable salt thereof, as provided herein can be determined by DC₅₀ value. As used herein, DC₅₀ refers to the concentration of the compound of Formula (I) or (II) that results in a 50% decrease in the concentration of a protein (e.g., BCL-X_{L} protein) in a cell compared to the concentration of the protein before the cell is contacted with the compound of Formula (I) or (II), or compared to the concentration of the protein in a cell not contacted with the compound of Formula (I) or (II). A compound with a lower DC₅₀ value, as determined under substantially similar conditions, is a more efficient inducer of degradation relative to a compound with a higher DC₅₀ value. In some embodiments, a DC₅₀ value can be determined (e.g., using HiBiT detection) in vitro or in vivo (e.g., in tumor cells (e.g., cell lines such as MOLT4, RS4;11, NCI-H146, EJM, HEK293T, HT1080, and/or H929) expressing a BCL-X_{L} protein).

Potency of degradation by a compound of Formula (I) or (II), or a pharmaceutically acceptable salt thereof, as provided herein can be determined by EC₅₀ value. As used herein, EC₅₀ refers to the concentration of the compound of Formula (I) or (II) that results in a 50% decrease in the concentration of a protein (e.g., BCL-X_{L} protein) relative to the trough concentration of the protein in a cell, when compared to the concentration of the protein before the cell is contacted with the compound of Formula (I) or (II), or compared to the concentration of the protein in a cell not contacted with the compound of Formula (I) or (II). A compound with a lower EC₅₀ value, as determined under substantially similar conditions, is a more potent compound relative to a compound with a higher EC₅₀ value. In some embodiments, an EC₅₀ value can be determined (e.g., using HiBiT detection) in vitro or in vivo (e.g., in tumor cells (e.g., cell lines such as MOLT4, RS4;11, NCI-H146, EJM, HEK293T, HT1080, and/or H929) expressing a BCL-X_{L} protein).

Potency of degradation by a compound of Formula (I) or (II), or a pharmaceutically acceptable salt thereof, as provided herein can be determined by Yₘᵢₙ value. As used herein, Yₘᵢₙ refers to the ratio of trough concentration of a protein (e.g., BCL-X_{L} protein) in a cell compared to the concentration of the protein before the cell is contacted with the compound of Formula (I) or (II), or compared to the concentration of the protein in a cell not contacted with the compound of Formula (I) or (II), expressed as a percentage. As used herein, Dmax is 1-Yₘᵢₙ. Yₘᵢₙ can be measured by a HiBiT assay as described in Example B1. A compound with a lower Yₘᵢₙ value, as determined under substantially similar conditions, is a more potent inducer of degradation relative to a compound with a higher Yₘᵢₙ value. A compound with a lower Yₘᵢₙ value, as determined under substantially similar conditions, is a more potent compound relative to a compound with a higher Yₘᵢₙ value. In some embodiments, a Yₘᵢₙ value can be determined (e.g., using HiBiT detection) in vitro or in vivo (e.g., in tumor cells (e.g., cell lines such as MOLT4, RS4;11, NCI-H146, EJM, HEK293T, HT1080, and/or H929) expressing a BCL-X_{L} protein).

An exemplary assay for determining the potency of a compound of Formula (I) or (II), or a pharmaceutically acceptable salt thereof, includes measuring the effect of the compound of Formula (I) or (II), or a pharmaceutically acceptable salt thereof, on cell proliferation and/or viability. Cell proliferation assays can be performed in a number of formats, including 2D and 3D. Similarly, a cell proliferation assay can be performed with any appropriate cell line, including, for example, MOLT4, RS4;11, NCI-H146, EJM, HEK293T, HT1080, and/or H929. As an illustrative example, a 3D cell proliferation assay can include growing cells in a 3D medium, contacting the cells with a compound of Formula (I) or (II), or a pharmaceutically acceptable salt thereof, measuring the cellular proliferation using an appropriate reagent (e.g., CELLTITERGLO® 3D), and then comparing the signal from an experiment with a compound of Formula (I) or (II), or a pharmaceutically acceptable salt thereof, to the signal from a control experiment (e.g., lacking the compound of Formula (I) or (II), or a pharmaceutically acceptable salt thereof). As another illustrative example, a 2D cell proliferation assay can include plating cells onto a growth surface, optionally letting the cells grow for a period of time, contacting the cells with a compound of Formula (I) or (II), or a pharmaceutically acceptable salt thereof, measuring the cellular proliferation using an appropriate reagent (e.g., CELLTITERGLO®), and then comparing the signal from an experiment with compound of Formula (I) or (II), or a pharmaceutically acceptable salt thereof, to the signal from a control experiment (e.g., lacking a compound of Formula (I) or (II), or a pharmaceutically acceptable salt thereof). Additional cell viability assays include MTT assays, which are colorimetric assays based on the reduction of the tetrazolium dye MTT (3-(4,5-dimethylthiazol-2-yl)-2,5-diphenyltetrazolium bromide) to the insoluble purple formazan, and other similar assays based on related tetrazolium salts. See, for instance, Example B3 and Example B4.

A cell viability assay can be used to measure the effect of a compound of Formula (I) or (II), or a pharmaceutically acceptable salt thereof, on cell death. For example, cells expressing BCL-X_{L} protein (e.g., MOLT-4 cells) can be incubated with various concentrations of a compound of Formula (I) or (II), or a pharmaceutically acceptable salt thereof, then exposed to a detection reagent (e.g., a CELLTITER-GLO® Cell Viability Assay kit) to determine cell viability. An exemplary assay for evaluating the affinity of a compound of Formula (I) or (II), or a pharmaceutically acceptable salt thereof, includes using a competition assay with recombinant BCL-X_{L} protein. For example, purified recombinant affinity-tagged (e.g., His-tagged) BCL-X_{L} protein can be incubated with various concentrations of a compound of Formula (I) or (II), or a pharmaceutically acceptable salt thereof, and a fixed concentration of affinity-tagged (e.g., biotin-tagged) BAD protein. After a period of incubation, FRET acceptor beads with a complementary affinity tag (e.g., His-acceptor beads) and FRET donor beads (e.g., streptavidin-tagged donor beads) can be added to the mixture, and a FRET reaction can be used to determine an inhibition constant of the compound of Formula (I) or (II), or pharmaceutically acceptable salt thereof. For example, an AlphaLISA competitive assay can be performed. See, e.g., International Publication No. WO 2019/144117.

An exemplary assay for determining the mechanism of cell death using of a compound of Formula (I) or (II), or a pharmaceutically acceptable salt thereof, includes measuring the effect of the compound of Formula (I) or (II), or a pharmaceutically acceptable salt thereof, on one or more markers of a mechanism of cell death (e.g., apoptosis). Exemplary markers of apoptosis include caspase induction (e.g., caspase 3/7 induction) and annexin V staining. Such assays may also be used as determinants of cell viability. For example, cells expressing BCL-X_{L} protein (e.g., MOLT-4 cells) can be incubated with various concentrations of a compound of Formula (I) or (II), or a pharmaceutically acceptable salt thereof, and relative caspase activity can be assessed using a luciferase substrate that is activated by caspase 3/7 (e.g., using the CASPASE-GLO® 3/7 assay). As another example, cells expressing BCL-X_{L} protein (e.g., MOLT-4 cells) can be incubated with various concentrations of a compound of Formula (I) or (II), or a pharmaceutically acceptable salt thereof, and relative caspase activity can be assessed using a dye that is coupled to an activated caspase motif (e.g., INCUCYTE® Caspase 3/7 Green Apoptosis Assay Reagent), followed by analysis using a platform for live cell imaging (e.g., an INCUCYTE® SX5 Live-Cell Analysis Instrument). As another example, cells expressing BCL-X_{L} protein (e.g., MOLT-4 cells) can be incubated with various concentrations of a compound of Formula (I) or (II), or a pharmaceutically acceptable salt thereof, and annexin V positivity can be assessed using phosphatidylserine dye (e.g., an INCUCYTE® Annexin V dye), followed by analysis using a platform for live cell imaging (e.g., an INCUCYTE® SX5 Live-Cell Analysis Instrument). See, for instance, Example B5.

As another example, the potency and/or efficacy of a compound of Formula (I) or (II), or a pharmaceutically acceptable salt thereof, can be evaluated in an animal model, for example, a xenograft model (e.g., using an established cancer cell line such as MOLT4, HEL, TF1, F36P, OCI-M1, OCI-M2, SET-2, CMK, M07E, or UKE-1, or a patient-derived xenograft (PDX) model). For example, a PDX model can be run in immunodeficient mice (e.g., athymic nude, outbred homozygous (e.g., Crl:NU(NCr)-Foxnl¹ⁿ) or Fox Chase SCID (CB17/Icr-Prkdcsci^{d}/IcrIcoCrl), mice). The mice can be female, 6-12 weeks old at tumor implantation and have access to food and water ad libitum. Approximately 70 mg of a tumor can be implanted subcutaneously in the right flank of each mouse. Following implantation, tumors can be measured weekly and once the tumor volumes reach 150-300 mm³, the mice can be randomized into treatment and control groups. In some embodiments, one or more experimental arms can be added to evaluate pharmacokinetics and/or pharmacodynamics. The mice can be treated with a compound of Formula (I) or (II), or a pharmaceutically acceptable salt thereof, (e.g., via IP or PO (oral) administration) and optionally an additional therapy or therapeutic agent (e.g., any of the additional therapies or therapeutic agents described herein). Throughout the study, health condition, body weight and tumor volumes of the mice can be recorded on a weekly basis. The mice can be sacrificed at 28 days or when the tumor reaches 1 cm³, and the tumors can be evaluated (e.g., by tumor weight, by tumor volume). At the end of each study, the Best Response can be calculated for each treatment arm. Best Response is defined as the minimum value of AVolumetfor t>10 days. Best Responses between the control arm(s) and the treatment arm(s) can be compared to determine if the treatment(s) work better than the control(s). In some embodiments, tumor samples can also be collected at the end of each study and relevant proteins (e.g., BCL-X_{L}, BCL-2, MCL-1, BIM, BAX, and/or BAK) can be measured to determine if the treatment might have a better protein modulation profile compared to a control. In some embodiments, tumor samples can also be collected at the end of each study and analyzed for signaling pathway activity (e.g., via phosphoERK levels). For pharmacokinetic and pharmacodynamic studies, tumor and/or blood samples from the mice can be obtained at the same or different time points than efficacy studies. For example, for pharmacokinetic and pharmacodynamic studies, tumor and/or blood samples from the mice can be obtained at Day 5, 6 hours post dosing, and relevant proteins can be measured in the tumor samples and pharmacokinetic studies can be performed on the blood samples or a portion thereof (e.g., plasma).

In some embodiments, the PDX is a model of a myeloproliferative neoplasm (MPN) (e.g., CEL, CML, CNL, essential thrombocythemia (e.g., JAK2 mutant (e.g., JAK2 V617F mutant) essential thrombocythemia or JAK2 wild type essential thrombocythemia), polycythemia vera (e.g., JAK2 mutant (e.g., JAK2 V617F mutant) polycythemia vera or JAK2 wild type polycythemia vera), or myelofibrosis (e.g., primary myelofibrosis (e.g., JAK2 mutant (e.g., JAK2 V617F mutant) primary myelofibrosis or JAK2 wild type primary myelofibrosis), post-essential thrombocythemia myelofibrosis (e.g., JAK2 mutant (e.g., JAK2 V617F mutant) post-essential thrombocythemia myelofibrosis or JAK2 wild type post-essential thrombocythemia myelofibrosis), or post-polycythemia vera myelofibrosis (e.g., JAK2 mutant (e.g., JAK2 V617F mutant) post-polycythemia vera myelofibrosis or JAK2 wild type post-polycythemia vera myelofibrosis)))), a CRC (e.g., BRaf mutant CRC (e.g., Braf V600E CRC) or KRas mutant CRC (e.g., KRas G12C mutant CRC or KRas G12D CRC)), SCLC (e.g., ASCL1 subtype SCLC or NEURODI subtype SCLC), a NSCLC (e.g., BRaf mutant NSCLC (e.g., Braf V600E NSCLC), an EGFR mutant NSCLC (e.g., EGFR L858R NSCLC or EGFR exon 19 deletion NSCLC), MET mutant NSCLC (e.g., MET exon 14 deletion NSCLC, MET amplified NSCLC), a KRas mutant NSCLC (e.g., KRas G12C NSCLC)), a lung squamous cell carcinoma, a malignant pleural mesothelioma (e.g., a BAP1 mutant malignant pleural mesothelioma), a melanoma (e.g., Braf mutant melanoma (e.g., Braf V600E melanoma)), a breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), a lymphoma (e.g., a T cell lymphoma (e.g., anaplastic large T cell lymphoma, cutaneous T cell lymphoma, or peripheral T cell lymphoma), or a non-Hodgkin lymphoma (e.g., DLBCL, anaplastic large T cell lymphoma, cutaneous T cell lymphoma, or peripheral T cell lymphoma), a leukemia (e.g., T cell leukemia (e.g., a T-ALL (e.g., relapsed/refractory T-ALL)), post-MPN leukemia, M6-AML, M7-AML), a head and neck cancer, a pancreatic cancer, a bladder cancer, an ovarian cancer (e.g., BRCA1 mutant ovarian cancer or BRCA2 mutant ovarian cancer, HGSOC (e.g., BRCA1 mutant HGSOC or BRCA2 mutant HGSOC)), a cervical cancer, an intrahepatic cholangiocarcinoma, or a mesenchymal cancer (e.g., mesenchymal breast cancer or mesenchymal kidney cancer).

See, e.g., Khan, et al. Nature Medicine 25.12 (2019): 1938-1947; Balachander, et al. Clinical Cancer Research 26.24 (2020): 6535-6549.

The pharmacokinetic parameters of a compound of Formula (I) or (II), or a pharmaceutically acceptable salt thereof, can be evaluated in an animal model, for instance, a mouse model, a rat model, a dog model, or a nonhuman primate (e.g., cynomolgus monkey) model. An exemplary assay includes the following. For example, pharmacokinetics (PK) studies can be conducted on animals (e.g., male or female CD-1 mice, Sprague Dawley rats, beagle dogs, or cynomolgus monkeys) by two delivery routes: intravenous (IV) injection and oral gavage (PO). Animals in both the IV and PO groups (e.g., n=3) are allowed free access to food and water. A compound of Formula (I) or (II), or a pharmaceutically acceptable salt thereof, can be formulated in solution for the IV route and solution or suspension for the PO route. On the day of the experiment, the compound of Formula (I) or (II), or a pharmaceutically acceptable salt thereof, can be administered via vein injection (e.g., at 1 mg/kg) for IV route or via oral gavage (e.g., at 5 to 10 mg/kg) for PO route. In some cases, the animals can be orally pre-dosed with a cytochrome P450 inhibitor (e.g., 1-aminobenzotriazole) prior to (e.g., 16 hours prior to) dosing the compound of Formula (I) or (II), or a pharmaceutically acceptable salt thereof. Blood samples can be collected via serial bleeding (e.g., at 8 timepoints from 0.83 to 24 hours post dose). At each timepoint, blood can be collected (e.g., approximately 30 μL of blood/timepoint) in a K₂EDTA tube via a vein (e.g., the saphenous vein). Blood samples can be put on wet ice and centrifuged (e.g., at 4600 RPM for 4 minutes) to obtain plasma samples. Plasma samples can be diluted (e.g., with an equal volume of pH 3.0 phosphate buffer) and submitted to LC-MS/MS for sample analysis. Pharmacokinetic parameters, including clearance (IV or PO, depending on the mode of dosing), area under the curve (AUC), and oral bioavailability (% F) can be calculated using a non-compartmental model.

In some embodiments, the % F for a compound of Formula (I) or (II), or a pharmaceutically acceptable salt thereof, is at least 4%. In some embodiments, the % F for a compound of Formula (I) or (II), or a pharmaceutically acceptable salt thereof, is at least 10%. In some embodiments, the % F for a compound of Formula (I) or (II), or a pharmaceutically acceptable salt thereof, is at least 20%. In some embodiments, the % F for a compound of Formula (I) or (II), or a pharmaceutically acceptable salt thereof, is at least 30%. In some embodiments, the % F for a compound of Formula (I) or (II), or a pharmaceutically acceptable salt thereof, is at least 40%. In some embodiments, the % F for a compound of Formula (I) or (II), or a pharmaceutically acceptable salt thereof, is about 4% to about 80% (e.g., about 4% to about 60%, about 4% to about 40%, about 4% to about 20%, about 4% to about 10%, about 20% to about 40%, or about 20% to about 30%). In some embodiments, the % F for a compound of Formula (I) or (II), or a pharmaceutically acceptable salt thereof, is about 4% to about 20%. In some embodiments, the % F for a compound of Formula (I) or (II), or a pharmaceutically acceptable salt thereof, is about 20% to about 40%. In some embodiments, the % F for a compound of Formula (I) or (II), or a pharmaceutically acceptable salt thereof, is about 40% to about 60%. In some embodiments, the % F for a compound of Formula (I) or (II), or a pharmaceutically acceptable salt thereof, is about 60% to about 80%.

In some embodiments, the clearance for a compound of Formula (I) or (II), or a pharmaceutically acceptable salt thereof, dosed PO in rats at 10 mg/kg, is less than 10 mL/min/kg (e.g., less than 5 mL/min/kg, less than 3 mL/min/kg, or less than 1 mL/min/kg). In some embodiments, clearance for a compound of Formula (I) or (II), or a pharmaceutically acceptable salt thereof, dosed PO in rats at 10 mg/kg, is about 0.05 mL/min/kg to about 5 mL/min/kg (e.g., about 0.05 mL/min/kg to about 3 mL/min/kg, about 0.05 mL/min/kg to about 1 mL/min/kg, or about 0.05 mL/min/kg to about 0.5 mL/min/kg).

In some embodiments, the AUC for a compound of Formula (I) or (II), or a pharmaceutically acceptable salt thereof, dosed PO in rats at 10 mg/kg, is about 10 μM·h to about 150 μM·h (e.g., about 10 μM·h to about 100 μM·h, about 10 μM·h to about 50 μM·h, or about 30 μM·h to about 80 μM·h).

Heterobifunctional degraders can, in some cases, induce the degradation of off-target proteins. For heterobifunctional degraders that utilize VHL, a common off-target proteins that can be degraded is CDO1. This degradation is generally believed to be due to the E3 binding moiety of the heterobifunctional degrader facilitating ternary complex formation between the off-target protein and VHL. CDO1 catalyzes the oxidation of cysteine to cysteine sulfinic acid, and undesired degradation may lead to nonspecific cytotoxicity.

In some embodiments, the compounds of Formula (I) or (II), or pharmaceutically acceptable salts thereof, can exhibit potent and selective induction of degradation of a BCL-X_{L} protein. In some embodiments, a compound of Formula (I) or (II), or a pharmaceutically acceptable salt thereof, or a pharmaceutically acceptable salt thereof, can selectively target a BCL-X_{L} protein for degradation over an off-target protein, such as another BCL-2 family member (e.g., BCL-2 and/or MCL-1) or non-BCL-2 family member target (e.g., CDO1).

As used herein, “selective” or “selectively”, when referring to a compound of Formula (I) or (II), or a pharmaceutically acceptable salt thereof in a protein degradation assay, indicates at least a 5-fold (e.g., at least a 10-fold, at least a 25-fold, at least a 50-fold, or at least a 100-fold) superior performance in the protein degradation assay for a specified protein with reference to a comparator protein in the assay. In some embodiments, the specified protein is BCL-X_{L} protein and the comparator is BCL-2 protein. For example, if a compound of Formula (I) or (II), or a pharmaceutically acceptable salt thereof, “selectively” induces degradation of BCL-X_{L} protein over BCL-2 protein as determined by a degradation assay, then the compound has at least a 5-fold (e.g., at least a 10-fold, at least a 25-fold, at least a 50-fold, or at least a 100-fold) smaller DC₅₀ value for BCL-X_{L} protein than for the BCL-2 protein when measured by the degradation assay.

In some embodiments, the compounds provided herein can exhibit potency (e.g., nanomolar potency) against a BCL-X_{L} protein with minimal activity (e.g., micromolar potency) against BCL-2 family members (e.g., BCL-2 or MCL-1 proteins). In some embodiments, a compound of Formula (I) or (II), or a pharmaceutically acceptable salt thereof, can exhibit potent degradation of a BCL-X_{L} protein and have minimal potency in degrading (e.g., as measured by Yₘᵢₙ, DC₅₀, and/or Dₘₐₓ values) an off-target protein (e.g., a BCL-2 family member (e.g., BCL-2 and/or MCL-1), and/or CDO1). In some embodiments, a compound of Formula (I) or (II), or a pharmaceutically acceptable salt thereof, can exhibit greater induction of degradation of a BCL-X_{L} protein relative to induction of degradation (e.g., as measured by Yₘᵢₙ, DC₅₀, and/or Dmax values) of an off-target protein (e.g., a BCL-2 family member (e.g., BCL-2 and/or MCL-1), and/or CDO1). In some embodiments, a compound of Formula (I) or (II), or a pharmaceutically acceptable salt thereof, can exhibit at least 2-fold, 3-fold, 5-fold, 10-fold, 25-fold, 50-fold, or 100-fold greater induction of degradation of a BCL-X_{L} protein relative to induction of degradation of an off-target protein. In some embodiments, a compound of Formula (I) or (II), or a pharmaceutically acceptable salt thereof, can exhibit up to 1000-fold greater induction of degradation of a BCL-X_{L} protein relative to induction of degradation of an off-target protein. In some embodiments, a compound of Formula (I) or (II), or a pharmaceutically acceptable salt thereof, can exhibit from about 2-fold to about 10-fold greater induction of degradation of a BCL-X_{L} protein relative to induction of degradation of an off-target protein (e.g., CDO1) (e.g., as measured by Yₘᵢₙ, DC₅₀, and/or Dmx values). In some embodiments, a compound of Formula (I) or (II), or a pharmaceutically acceptable salt thereof, can exhibit from about 10-fold to about 100-fold greater induction of degradation of a BCL-X_{L} protein relative to induction of degradation of an off-target protein. In some embodiments, a compound of Formula (I) or (II), or a pharmaceutically acceptable salt thereof, can exhibit from about 100-fold to about 1000-fold greater induction of degradation of a BCL-X_{L} protein relative to induction of degradation of an off-target protein. In some embodiments, a compound of Formula (I) or (II), or a pharmaceutically acceptable salt thereof, can exhibit from about 1000-fold to about 10000-fold greater induction of degradation of a BCL-X_{L} protein relative to induction of degradation of an off-target protein.

Certain agents that inhibit or induce degradation of BCL-X_{L} have demonstrated platelet toxicity, which resulted in dose-limiting toxicity (e.g., thrombocytopenia) in the clinic. See, e.g., Adams and Cory, Cell Death & Differentiation 25.1 (2018): 27-36; Campbell and Tait. Open Biology 8.5 (2018): 180002; Pullarkat et al., Cancer Discovery (2021) 10.1158/2159-8290.CD-20-1465; Negi and Voisin-Chiret. ChemBioChem (2022) (doi: 10.1002/cbic.202100689). The viability of platelets may be monitored with any appropriate assay, such as those described herein. See, for instance, Example B6.

In some embodiments, a therapeutically effective amount of a compound of Formula (I) or (II), or a pharmaceutically acceptable salt thereof, shows at least about 30% platelet viability (e.g., at least about 50% platelet viability, or at least about 80% platelet viability) when administered to a subject. In some embodiments, a therapeutically effective amount of a compound of Formula (I) or (II), or a pharmaceutically acceptable salt thereof, shows about 30% to about 100% platelet viability (e.g., about 50% to about 100% platelet viability, or about 80% to about 100% platelet viability) when administered to a subject.

In some embodiments, a therapeutically effective amount of a compound of Formula (I) or (II), or a pharmaceutically acceptable salt thereof, shows at least about 30% platelet viability (e.g., at least about 50% platelet viability, or at least about 80% platelet viability) when administered to a subject and has a Yₘᵢₙ value of about 50% to about 70%. In some embodiments, a therapeutically effective amount of a compound of Formula (I) or (II), or a pharmaceutically acceptable salt thereof, shows at least about 30% platelet viability (e.g., at least about 50% platelet viability, or at least about 80% platelet viability) when administered to a subject and has a Yₘᵢₙ value of less than about 50%. In some embodiments, a therapeutically effective amount of a compound of Formula (I) or (II), or a pharmaceutically acceptable salt thereof, shows at least about 30% platelet viability (e.g., at least about 50% platelet viability, or at least about 80% platelet viability) when administered to a subject and has a Yₘᵢₙ value of about 0% to about 50%.

In some embodiments, a therapeutically effective amount of a compound of Formula (I) or (II), or a pharmaceutically acceptable salt thereof, shows between about 30% and 100% platelet viability (e.g., about 50% to about 100% platelet viability, or about 80% to about 100% platelet viability) when administered to a subject and has a Yₘᵢₙ value of about 50% to about 70%. In some embodiments, a therapeutically effective amount of a compound of Formula (I) or (II), or a pharmaceutically acceptable salt thereof, shows between about 30% and 100% platelet viability (e.g., about 50% to about 100% platelet viability, or about 80% to about 100% platelet viability) when administered to a subject and has a Yₘᵢₙ value of less than about 50%. In some embodiments, a therapeutically effective amount of a compound of Formula (I) or (II), or a pharmaceutically acceptable salt thereof, shows between about 30% and 100% platelet viability (e.g., about 50% to about 100% platelet viability, or about 80% to about 100% platelet viability) when administered to a subject and has a Yₘᵢₙ value of about 0% to about 50%.

In some embodiments, therapeutically effective amount of a compound of Formula (I) or (II), or a pharmaceutically acceptable salt thereof, shows at least about 50% platelet viability (e.g., at least about 80% platelet viability) when administered to a subject and has a Yₘᵢₙ value of about 50% to about 70%. In some embodiments, a therapeutically effective amount of a compound of Formula (I) or (II), or a pharmaceutically acceptable salt thereof, shows at least about 50% platelet viability (e.g., at least about 80% platelet viability) when administered to a subject and has a Yₘᵢₙ value of less than about 50%. In some embodiments, a therapeutically effective amount of a compound of Formula (I) or (II), or a pharmaceutically acceptable salt thereof, shows at least about 50% platelet viability (e.g., at least about 80% platelet viability) when administered to a subject and has a Yₘᵢₙ value of about 0% to about 50%.

In some embodiments, a therapeutically effective amount of a compound of Formula (I) or (II), or a pharmaceutically acceptable salt thereof, shows between about 50% and 100% platelet viability (e.g., about 80% to about 100% platelet viability) when administered to a subject and has a Yₘᵢₙ value of about 50% to about 70%. In some embodiments, a therapeutically effective amount of a compound of Formula (I) or (II), or a pharmaceutically acceptable salt thereof, shows between about 50% and 100% platelet viability (e.g., about 80% to about 100% platelet viability) when administered to a subject and has a Yₘᵢₙ value of less than about 50%. In some embodiments, a therapeutically effective amount of a compound of Formula (I) or (II), or a pharmaceutically acceptable salt thereof, shows between about 50% and 100% platelet viability (e.g., about 80% to about 100% platelet viability) when administered to a subject and has a Yₘᵢₙ value of about 0% to about 50%.

In some embodiments, a compound of Formula (I) or (II), or a pharmaceutically acceptable salt thereof, when tested at a concentration of about 0.25 μM to about 3 μM in the assay in Example B6, shows at least about 30% platelet viability (e.g., at least about 50% platelet viability, or at least about 80% platelet viability). In some embodiments, a compound of Formula (I) or (II), or a pharmaceutically acceptable salt thereof, when tested at a concentration of about 0.25 μM to about 3 μM in the assay in Example B6, shows about 30% to about 100% platelet viability (e.g., about 50% to about 100% platelet viability, or about 80% to about 100% platelet viability).

In some embodiments, a compound of Formula (I) or (II), or a pharmaceutically acceptable salt thereof, when tested at a concentration of about 0.25 μM to about 3 μM in the assay in Example B6, shows at least about 30% platelet viability (e.g., at least about 50% platelet viability, or at least about 80% platelet viability) and has a Yₘᵢₙ value of about 50% to about 70% in the assay described in Example B1. In some embodiments, a compound of Formula (I) or (II), or a pharmaceutically acceptable salt thereof, when tested at a concentration of about 0.25 μM to about 3 μM in the assay in Example B6, shows at least about 30% platelet viability (e.g., at least about 50% platelet viability, or at least about 80% platelet viability) and has a Yₘᵢₙ value of less than about 50% in the assay described in Example B1. In some embodiments, a compound of Formula (I) or (II), or a pharmaceutically acceptable salt thereof, when tested at a concentration of about 0.25 μM to about 3 μM in the assay in Example B6, shows at least about 30% platelet viability (e.g., at least about 50% platelet viability, or at least about 80% platelet viability) and has a Yₘᵢₙ value of about 0% to about 50% in the assay described in Example B1.

In some embodiments, a compound of Formula (I) or (II), or a pharmaceutically acceptable salt thereof, when tested at a concentration of about 0.25 μM to about 3 μM in the assay in Example B6, shows between about 30% and 100% platelet viability (e.g., about 50% to about 100% platelet viability, or about 80% to about 100% platelet viability) and has a Yₘᵢₙ value of about 50% to about 70% in the assay described in Example B1. In some embodiments, a compound of Formula (I) or (II), or a pharmaceutically acceptable salt thereof, when tested at a concentration of about 0.25 μM to about 3 μM in the assay in Example B6, shows between about 30% and 100% platelet viability (e.g., about 50% to about 100% platelet viability, or about 80% to about 100% platelet viability) and has a Yₘᵢₙ value of less than about 50% in the assay described in Example B1. In some embodiments, a compound of Formula (I) or (II), or a pharmaceutically acceptable salt thereof, when tested at a concentration of about 0.25 μM to about 3 μM in the assay in Example B6, shows between about 30% and 100% platelet viability (e.g., about 50% to about 100% platelet viability, or about 80% to about 100% platelet viability) and has a Yₘᵢₙ value of about 0% to about 50% in the assay described in Example B1.

In some embodiments, a compound of Formula (I) or (II), or a pharmaceutically acceptable salt thereof, when tested at a concentration of about 0.25 μM to about 3 μM in the assay in Example B6, shows at least about 50% platelet viability (e.g., at least about 80% platelet viability) and has a Yₘᵢₙ value of about 50% to about 70% in the assay described in Example B1. In some embodiments, a compound of Formula (I) or (II), or a pharmaceutically acceptable salt thereof, when tested at a concentration of about 0.25 μM to about 3 μM in the assay in Example B6, shows at least about 50% platelet viability (e.g., at least about 80% platelet viability) and has a Yₘᵢₙ value of less than about 50% in the assay described in Example B1. In some embodiments, a compound of Formula (I) or (II), or a pharmaceutically acceptable salt thereof, when tested at a concentration of about 0.25 μM to about 3 μM in the assay in Example B6, shows at least about 50% platelet viability (e.g., at least about 80% platelet viability) and has a Yₘᵢₙ value of about 0% to about 50% in the assay described in Example B1.

In some embodiments, a compound of Formula (I) or (II), or a pharmaceutically acceptable salt thereof, when tested at a concentration of about 0.25 μM to about 3 μM in the assay in Example B6, shows between about 50% and 100% platelet viability (e.g., about 80% to about 100% platelet viability) and has a Yₘᵢₙ value of about 50% to about 70% in the assay described in Example B1. In some embodiments, a compound of Formula (I) or (II), or a pharmaceutically acceptable salt thereof, when tested at a concentration of about 0.25 μM to about 3 μM in the assay in Example B6, shows between about 50% and 100% platelet viability (e.g., about 80% to about 100% platelet viability) and has a Yₘᵢₙ value of less than about 50% in the assay described in Example B1. In some embodiments, a compound of Formula (I) or (II), or a pharmaceutically acceptable salt thereof, when tested at a concentration of about 0.25 μM to about 3 μM in the assay in Example B6, shows between about 50% and 100% platelet viability (e.g., about 80% to about 100% platelet viability) and has a Yₘᵢₙ value of about 0% to about 50% in the assay described in Example B1.

Provided herein is a method of treating a cancer in a subject in need of such treatment, the method comprising administering to the subject a therapeutically effective amount of a compound of Formula (I) or (II), or a pharmaceutically acceptable salt thereof, or a pharmaceutical composition thereof. In some embodiments, the subject is treatment naïve with respect to the cancer. In some embodiments, the subject has received one or more lines of previous therapy for the cancer.

Also provided herein is a method of treating a cancer in a subject in need thereof, the method comprising administering to the subject a therapeutically effective amount of a compound of Formula (I) or (II), or a pharmaceutically acceptable salt thereof, or a pharmaceutical composition thereof, as a monotherapy. In some embodiments, the subject is treatment naïve with respect to the cancer. In some embodiments, the subject has received one or more lines of previous therapy for the cancer.

Provided herein is a method of treating a cancer in a subject in need of such treatment, the method comprising:
(a) detecting a biomarker (e.g., a mutation, an amplification, a copy number increase, and/or expression (optionally including level of expression) of the biomarker) associated with the cancer; and
(b) administering to the subject a therapeutically effective amount of a compound of Formula (I) or (II), or a pharmaceutically acceptable salt thereof, or a pharmaceutical composition thereof, as a monotherapy or in combination with an additional therapy or therapeutic agent.

Also provided herein is a method of treating a cancer in a subject, wherein the subject has been determined (e.g., prior to administration of a compound of Formula (I) or (II)) to have a biomarker (e.g., a mutation, an amplification, a copy number increase, and/or expression (optionally including level of expression) of the biomarker) associated with the cancer, the method comprising administering to the subject a therapeutically effective amount of a compound of Formula (I) or (II), or a pharmaceutically acceptable salt thereof, or a pharmaceutical composition thereof, as a monotherapy or in combination with an additional therapy or therapeutic agent.

Provided herein is a method of treating a cancer in a subject in need of such treatment, the method comprising:
(a) detecting a biomarker (e.g., a mutation, an amplification, a copy number increase, and/or expression (optionally including level of expression) of a marker of susceptibility to particular agents (e.g., HER2 expression, ER expression, PR expression, folate receptor expression)) associated with the cancer; and
(b) administering to the subject a therapeutically effective amount of a compound of Formula (I) or (II), or a pharmaceutically acceptable salt thereof, or a pharmaceutical composition thereof, as a monotherapy or in combination with an additional therapy or therapeutic agent.

Also provided herein is a method of treating a cancer in a subject, wherein the subject has been determined (e.g., prior to administration of a compound of Formula (I) or (II)) to have a biomarker (e.g., a mutation, an amplification, a copy number increase, and/or expression (optionally including level of expression) of a marker of susceptibility to particular agents (e.g., HER2 expression, ER expression, PR expression, folate receptor expression)) associated with the cancer, the method comprising administering to the subject a therapeutically effective amount of a compound of Formula (I) or (II), or a pharmaceutically acceptable salt thereof, or a pharmaceutical composition thereof, as a monotherapy or in combination with an additional therapy or therapeutic agent.

Provided herein is use of a compound of Formula (I) or (II), or a pharmaceutically acceptable salt thereof, or a pharmaceutical composition thereof, for the treatment of cancer, for example, any of the cancers provided herein.

Provided herein is use of a compound of Formula (I) or (II), or a pharmaceutically acceptable salt thereof, or a pharmaceutical composition thereof, as a medicament for the treatment of cancer, for example, any of the cancers provided herein.

Provided herein is use of a compound of Formula (I) or (II), or a pharmaceutically acceptable salt thereof, in the manufacture of a medicament for the treatment of cancer, for example, any of the cancers provided herein.

Provided herein is a compound of Formula (I) or (II), or a pharmaceutically acceptable salt thereof, or a pharmaceutical composition thereof, for use as a medicament. Also provided herein is a compound of Formula (I) or (II), or a pharmaceutically acceptable salt thereof, or a pharmaceutical composition thereof, for use as a medicament for the treatment of cancer, for example, any of the cancers provided herein.

Provided herein is a compound of Formula (I) or (II), or a pharmaceutically acceptable salt thereof, or a pharmaceutical composition thereof, for use in treating a cancer, for example, any of the cancers provided herein.

As used herein, “monotherapy”, when referring to a compound of Formula (I) or (II), or a pharmaceutically acceptable salt thereof, means that the compound of Formula (I) or (II), or a pharmaceutically acceptable salt thereof, is the only therapeutic agent or therapy (e.g., anticancer agent or therapy) administered to the subject during the treatment cycle (e.g., no additional targeted therapeutics, anticancer agents, chemotherapeutics, or checkpoint inhibitors are administered to the subject during the treatment cycle). As a person of ordinary skill in the art would understand, monotherapy does not exclude the co-administration of medicaments for the treatment of side effects or general symptoms associated with the cancer or treatment, such as pain, rash, edema, photosensitivity, pruritis, skin discoloration, hair brittleness, hair loss, brittle nails, cracked nails, discolored nails, swollen cuticles, fatigue, weight loss, general malaise, shortness of breath, infection, anemia, or gastrointestinal symptoms, including nausea, diarrhea, and lack of appetite.

As used herein, “the subject has previously received one or more therapeutic agents or therapies for the cancer” means that the subject has been previously administered one or more therapeutic agents or therapies (e.g., anticancer agent or therapy) for the cancer other than a compound of Formula (I) or (II), or a pharmaceutically acceptable salt thereof, during a prior treatment cycle. In some embodiments, the subject cannot tolerate the one or more therapeutic agents or therapies previously administered for the cancer. In some embodiments, the subject did not respond to the one or more therapeutic agents or therapies previously administered for the cancer. In some embodiments, the subject did not adequately respond to one or more therapeutic agents or therapies previously administered for the cancer. In some embodiments, the subject has stopped responding to the one or more therapeutic agents or therapies previously administered for the cancer. In some embodiments, a lack of response, an inadequate response, or a discontinued response can be determined by objective criteria (e.g., tumor volume, or by criteria such as RECIST 1.1). In some embodiments, a lack of response, an inadequate response, or a discontinued response can be determined by the subject's physician.

As used herein, “the subject is treatment naïve with respect to the cancer” means that the subject has not been previously administered one or more therapeutic agents or therapies for the cancer.

For any of the solid tumors described herein, the solid tumor can be primary tumors or metastatic (or secondary) tumors. As used herein, “primary” tumors are those located at the site where the tumor began to grow (i.e., where it originated). As used herein, “metastatic” (or “secondary”) tumors are those that have spread to other parts of body from the original tumor site. In some embodiments, the metastatic or secondary tumors are the same type of cancer as the primary tumor. In some embodiments, the metastatic or secondary tumors are not genetically identical to the primary tumor.

In some embodiments of any of the methods or uses described herein, the cancer is breast cancer (e.g., breast invasive carcinoma, breast invasive ductal carcinoma), central or peripheral nervous system tissue cancer (e.g., brain cancer (e.g., astrocytoma, glioblastoma, glioma, oligoastrocytoma)), endocrine or neuroendocrine cancer (e.g., adrenal cancer (e.g., adrenocortical carcinoma, neuroblastoma, pheochromocytoma, paraganglioma), multiple neuroendocrine type I and type II tumors, parathyroid cancer, pituitary tumors, thyroid cancer (e.g., papillary thyroid cancer)), eye cancer (e.g., uveal cancer (e.g., uveal melanoma)), gastrointestinal cancer (e.g., anal cancer, bile duct cancer (e.g., cholangiocarcinoma (e.g., intrahepatic cholangiocarcinoma)), colorectal cancer (e.g., colon adenocarcinoma, rectal adenocarcinoma, mucinous adenocarcinoma, mucinous carcinoma), esophageal cancer (e.g., esophageal adenocarcinoma), gallbladder cancer, gastrointestinal carcinoid tumor, gastrointestinal stromal tumor (GIST), liver cancer (e.g., hepatocellular carcinoma, intrahepatic bile duct cancer), pancreatic cancer (e.g., pancreatic adenocarcinoma, pancreatic islet cell cancer), small intestine cancer, or stomach cancer (e.g., stomach adenocarcinoma, signet ring cell carcinoma of the stomach)), genitourinary cancer (e.g., bladder cancer (e.g., bladder urothelial carcinoma), kidney cancer (e.g., renal clear cell carcinoma, renal papillary cell carcinoma, kidney chromophobe), prostate cancer (e.g., prostate adenocarcinoma), testicular cancer (e.g., testicular germ cell tumors), or ureter cancer), gynecologic cancer (e.g., cervical cancer (e.g., cervical squamous cell carcinoma, endocervical adenocarcinoma, mucinous carcinoma), ovarian cancer (e.g., serous ovarian cancer, ovarian serous cystadenocarcinoma), uterine cancer (e.g., uterine carcinosarcoma, uterine endometrioid carcinoma, uterine serous carcinoma, uterine papillary serous carcinoma, uterine corpus endometrial carcinoma), or vulvar cancer), head and neck cancer (e.g., ear cancer (e.g., middle ear cancer), head and neck squamous cell carcinoma, nasal cavity cancer, oral cancer, pharynx cancer (e.g., hypopharynx cancer, nasopharynx cancer, oropharyngeal cancer), hematological cancer (e.g., leukemia (e.g., chronic eosinophilic leukemia (CEL), chronic lymphocytic leukemia (CLL), chronic neutrophilic leukemia (CNL), acute lymphocytic leukemia (ALL) (e.g., Philadelphia chromosome positive ALL or T cell ALL (T-ALL)), acute myeloid leukemia (AML) (e.g., acute promyelocytic leukemia (APL), post-MPN AML, post-myelodysplastic syndrome (post-MDS) AML, M6-AML (also known as pure erythroid leukemia (PEL)), or M7-AML (also known as acute megakaryoblastic leukemia (AKML))), chronic myeloid leukemia (CML)), lymphoma (e.g., Hodgkin lymphoma (e.g., nodular lymphocyte predominant Hodgkin lymphoma (NLPHL)), or non-Hodgkin lymphoma (e.g., Burkitt lymphoma (BL), diffuse large B cell lymphoma (DLBCL), diffuse histiocytic lymphoma (DHL), follicular lymphoma (FL), intravascular large B cell lymphoma (IVLBCL), mantle cell lymphoma (MCL), small lymphocytic lymphoma (SLL), a T cell lymphoma (e.g., anaplastic large T cell lymphoma, cutaneous T cell lymphoma, or peripheral T cell lymphoma))), essential thrombocythemia, polycythemia vera, myelofibrosis (e.g., primary myelofibrosis, post-essential thrombocythemia myelofibrosis, or post-polycythemia vera myelofibrosis), a myelodysplastic syndrome (MDS) (e.g., M6 MDS or M7 MDS), or myeloma (e.g., multiple myeloma)), Li-Fraumeni tumors, mesentery cancer (e.g., omentum cancer, peritoneal cancer), pleural cancer, respiratory cancer (e.g., larynx cancer, lung cancer (e.g., lung squamous cell carcinoma, lung adenocarcinoma, malignant pleural mesothelioma, non-small cell lung cancer (NSCLC), small cell lung cancer), tracheal cancer), sarcoma (e.g., bone cancer (e.g., osteosarcoma, chondrosarcoma) or soft tissue sarcoma (Ewing sarcoma, leiomyosarcoma, myxofibrosarcoma, rhabdomyosarcoma)), skin cancer (e.g., melanoma or Merkel cell carcinoma), thymus cancer (e.g., thymoma), or a combination thereof.

In some embodiments, the cancer is breast cancer, gastrointestinal cancer (e.g., bile duct cancer (e.g., cholangiocarcinoma (e.g., intrahepatic cholangiocarcinoma)), colorectal cancer (CRC), gastrointestinal stromal tumor, or pancreatic cancer), genitourinary cancer (e.g., bladder cancer (e.g., bladder urothelial carcinoma) or kidney cancer), gynecologic cancer (e.g., cervical cancer, ovarian cancer (e.g., high grade serous ovarian cancer (HGSOC), low grade serous ovarian cancer (LGSOC)), or uterine cancer), head and neck cancer (e.g., head and neck squamous cell carcinoma), hematological cancer (e.g., leukemia (e.g., acute lymphocytic leukemia (ALL) (e.g., T-ALL), acute myeloid leukemia (AML) (e.g., APL, post-MPN AML, post-MDS AML, M6-AML, or M7-AML), chronic lymphocytic leukemia (CLL)), lymphoma (e.g., follicular lymphoma (FL), small lymphocytic lymphoma (SLL), a T cell lymphoma (e.g., anaplastic large T cell lymphoma, cutaneous T cell lymphoma, or peripheral T cell lymphoma), or diffuse large B cell lymphoma (DLBCL)), essential thrombocythemia, polycythemia vera, myelofibrosis (e.g., primary myelofibrosis, post-essential thrombocythemia myelofibrosis, or post-polycythemia vera myelofibrosis), or a myelodysplastic syndrome (MDS) (e.g., M6 MDS or M7 MDS)), lung cancer (e.g., non small cell lung cancer (NSCLC), small cell lung cancer (SCLC), lung squamous cell carcinoma, or malignant pleural mesothelioma (MPM)), neuroblastoma, sarcoma (e.g., bone cancer (e.g., osteosarcoma, chondrosarcoma) or soft tissue sarcoma (Ewing sarcoma, leiomyosarcoma, myxofibrosarcoma, rhabdomyosarcoma)), or skin cancer (e.g., melanoma or Merkel cell carcinoma).

In some embodiments, the cancer is a myeloproliferative neoplasm (MPN). In some embodiments, the myeloproliferative neoplasm is CEL, CML, CNL, essential thrombocythemia, polycythemia vera, or myelofibrosis (e.g., primary myelofibrosis, post-essential thrombocythemia myelofibrosis, or post-polycythemia vera myelofibrosis). In some embodiments, the MPN has a JAK2 mutation (e.g., a JAK2 V617F mutation). In some embodiments, the MPN does not have a JAK2 mutation. In some embodiments, the cancer is low-risk myelofibrosis. In some embodiments, the cancer is intermediate (e.g., intermediate-1 and/or intermediate-2) or high-risk myelofibrosis (e.g., primary myelofibrosis, post-essential thrombocythemia myelofibrosis, or post-polycythemia vera myelofibrosis). In some embodiments, the cancer is intermediate or high-risk myelofibrosis (e.g., primary myelofibrosis, post-essential thrombocythemia myelofibrosis, or post-polycythemia vera myelofibrosis) with a JAK2 mutation (e.g., a JAK2 V617F mutation). In some embodiments, the cancer is intermediate or high-risk myelofibrosis (e.g., primary myelofibrosis, post-essential thrombocythemia myelofibrosis, or post-polycythemia vera myelofibrosis) without a JAK2 V617F mutation. In some embodiments, the subject has a platelet count below 50×10⁹/L.

In some embodiments, the cancer is polycythemia vera. In some embodiments, the cancer is polycythemia vera, and the subject has had an inadequate response to or is intolerant of hydroxyurea.

In some embodiments, the cancer is an MDS. In some embodiments, the cancer is M6 MDS. In some embodiments, the cancer is M7 MDS.

In some embodiments, the cancer is T-ALL. In some embodiments, the cancer is relapsed/refractory T-ALL.

In some embodiments, the cancer is CRC (e.g., Braf mutant CRC (e.g., Braf V600E CRC) or KRas mutant CRC (e.g., KRas G12C CRC or KRas G12D CRC)).

In some embodiments, the cancer is SCLC (e.g., ASCL1 subtype SCLC or NEUROD1 subtype SCLC).

In some embodiments, the cancer is NSCLC (e.g., Braf mutant NSCLC (e.g., Braf V600E NSCLC), EGFR mutant NSCLC (e.g., EGFR L858R NSCLC or EGFR exon 19 deletion NSCLC), MET mutant NSCLC (e.g., MET exon 14 deletion NSCLC, MET amplified NSCLC), KRas mutant NSCLC (e.g., KRas G12C NSCLC)).

In some embodiments, the cancer is lung squamous cell carcinoma.

In some embodiments, the cancer is malignant pleural mesothelioma (e.g., BAP1 mutant malignant pleural mesothelioma). In some embodiments, the cancer is malignant pleural mesothelioma, and a compound of Formula (I) or (II), or a pharmaceutically acceptable salt thereof, is administered as a monotherapy.

In some embodiments, the cancer is melanoma (e.g., Braf mutant melanoma (e.g., Braf V600E melanoma)).

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), HER2 low breast cancer, triple negative breast cancer, hormone receptor positive breast cancer (ER+ and/or PR+, with or without HER2 positivity)).

In some embodiments, the cancer is lymphoma. In some embodiments, the lymphoma is a T cell lymphoma (e.g., anaplastic large T cell lymphoma, cutaneous T cell lymphoma, or peripheral T cell lymphoma). In some embodiments, the lymphoma is a non-Hodgkin lymphoma (e.g., DLBCL, anaplastic large T cell lymphoma, cutaneous T cell lymphoma, or peripheral T cell lymphoma). In some embodiments, the lymphoma is peripheral T cell lymphoma.

In some embodiments, the cancer is leukemia. In some embodiments, the leukemia is a T cell leukemia (e.g., T cell ALL). In some embodiments, the cancer is post-MPN leukemia.

In some embodiments, the cancer is a T cell leukemia (e.g., T cell ALL), and a compound of Formula (I) or (II), or a pharmaceutically acceptable salt thereof, is administered as a monotherapy.

In some embodiments, the cancer is M6-AML. In some embodiments, the M6-AML is a post-MPN AML. In some embodiments, the M6-AML is a post-myelodysplastic syndrome (MDS) AML. In some embodiments, the cancer is M6-AML, and the compound of Formula (I) or (II), or pharmaceutically acceptable salt thereof, is administered as a monotherapy.

In some embodiments, the cancer is M7-AML. In some embodiments, the M7-AML is a post-MPN AML. In some embodiments, the M7-AML is a post-MDS AML. In some embodiments, the cancer is M7-AML, and the compound of Formula (I) or (II), or pharmaceutically acceptable salt thereof, is administered as a monotherapy.

In some embodiments, the cancer is head and neck cancer.

In some embodiments, the cancer is essential thrombocythemia.

In some embodiments, the cancer is polycythemia vera.

In some embodiments, the cancer is myelofibrosis. In some embodiments, the cancer is primary myelofibrosis. In some embodiments, the cancer is post-essential thrombocythemia myelofibrosis. In some embodiments, the cancer is post-polycythemia vera myelofibrosis.

In some embodiments, the cancer is an MDS. In some embodiments, the MDS is M6 MDS. IN some embodiments, the MDS is M7 MDS. In some embodiments, the cancer is an MDS, and a compound of Formula (I) or (II), or a pharmaceutically acceptable salt thereof, is administered as a monotherapy.

In some embodiments, the cancer is pancreatic cancer.

In some embodiments, the cancer is bladder cancer (e.g., bladder urothelial carcinoma).

In some embodiments, the cancer is ovarian cancer (e.g., BRCA1 mutant ovarian cancer or BRCA2 mutant ovarian cancer). In some embodiments, the cancer is HGSOC (e.g., BRCA1 mutant HGSOC or BRCA2 mutant HGSOC).

In some embodiments, the cancer is cervical cancer.

In some embodiments, the cancer is colorectal cancer.

In some embodiments, the cancer is skin cancer. In some embodiments, the skin cancer is melanoma. In some embodiments, the cancer is Merkel cell carcinoma. In some embodiments, the cancer is Merkel cell carcinoma, and a compound of Formula (I) or (II), or a pharmaceutically acceptable salt thereof, is administered as a monotherapy.

In some embodiments, the cancer is neuroblastoma.

In some embodiments, the cancer is intrahepatic cholangiocarcinoma.

In some embodiments, the cancer is a mesenchymal cancer. In some embodiments, the mesenchymal cancer is mesenchymal breast cancer or mesenchymal kidney cancer.

In some embodiments, a BCL-X_{L} copy number gain or a BCL-X_{L} amplification can be detected in a sample from the subject (e.g., detecting three or more copies of a BCL2L1 gene in the sample from the subject). In some embodiments, the subject was determined (e.g., prior to administration of a compound of Formula (I) or (II)) to have a cancer having a BCL-X_{L} copy number gain or a BCL-X_{L} amplification.

In some embodiments, the cancer has a BCL-X_{L} copy number gain.

In some embodiments, the cancer has a BCL-X_{L} amplification.

Non-limiting examples of BCL-X_{L} involvement in cancers can be found in: Wilson, Wyndham H., et al. The Lancet Oncology 11.12 (2010): 1149-1159; Keitel, Ulrike, et al. Oncotarget 5.23 (2014): 11778; Chonghaile, Triona Ni, et al. Cancer Discovery 4.9 (2014): 1074-1087; Zaanan, Aziz, et al. Journal of Biological Chemistry 290.39 (2015): 23838-23849; Zhang, Haichao, et al. Molecular Cancer 14.1 (2015): 1-9; Soderquist, Ryan S., et al. Nature Communications 9.1 (2018): 1-13; Stover, Elizabeth H., et al. Molecular Cancer Research 17.11 (2019): 2281-2293; Concoran, R. B., et al. Annals of Oncology (2019) 30 (suppl_5): v159-v193; Lakhani, Nehal J., et al. Journal of Clinical Oncology (2020): 3509-3509 doi: 10.1200/JCO.2020.38; He, Yonghan, et al. Journal of Hematology & Oncology 13.1 (2020): 1-13; Grubb, Treg, et al. Clinical Cancer Research (2022) doi: 10.1158/1078-0432.CCR-22-0669; Joly, Florence, et al. Gynecologic Oncology 165.1 (2022): 30-39; and Nanjo, Shigeki, et al. The Journal of Clinical Investigation (2022) doi: 10.1172/JCI145099.

In some embodiments, the subject has previously been treated with another anticancer agent, a chemotherapeutic agent, radiation, surgery, a multi-kinase inhibitor, or a combination thereof.

Provided herein is a method of treating an ocular disease or condition in a subject in need of such treatment, the method comprising administering (e.g., intravitreally or topically) to the subject a therapeutically effective amount of a compound of Formula (I) or (II), or a pharmaceutically acceptable salt thereof, or a pharmaceutical composition thereof. In some embodiments, the ocular disease or condition is diabetic macular edema. In some embodiments, the ocular disease or condition is age-related macular degeneration. In some embodiments, the ocular disease or condition is diabetic retinopathy. See, e.g., Crespo-Garcia, Sergio, et al. Cell Metabolism 33.4 (2021): 818-832; Hassan, Jannah Waled, and Ashay D. Bhatwadekar, Frontiers in Pharmacology 13 (2022): 896907.

Provided herein is use of a compound of Formula (I) or (II), or a pharmaceutically acceptable salt thereof, or a pharmaceutical composition thereof, for the treatment of an ocular disease or condition, for example, any of the ocular diseases or conditions provided herein.

Provided herein is use of a compound of Formula (I) or (II), or a pharmaceutically acceptable salt thereof, or a pharmaceutical composition thereof, as a medicament for the treatment of an ocular disease or condition, for example, any of the ocular diseases or conditions provided herein.

Provided herein is use of a compound of Formula (I) or (II), or a pharmaceutically acceptable salt thereof, in the manufacture of a medicament for the treatment of an ocular disease or condition, for example, any of the ocular diseases or conditions provided herein.

Also provided herein is a compound of Formula (I) or (II), or a pharmaceutically acceptable salt thereof, or a pharmaceutical composition thereof, for use as a medicament for the treatment of an ocular disease or condition, for example, any of the ocular diseases or conditions provided herein.

Provided herein is a compound of Formula (I) or (II), or a pharmaceutically acceptable salt thereof, or a pharmaceutical composition thereof, for use in treating an ocular disease or condition, for example, any of the ocular diseases or conditions provided herein.

Also provided herein is a method of treating a fibrotic disease or condition and/or a disease or condition associated with senescent cells in a subject in need of such treatment, the method comprising administering to the subject a therapeutically effective amount of a compound of Formula (I) or (II), or a pharmaceutically acceptable salt thereof, or a pharmaceutical composition thereof. Non-limiting examples of fibrotic diseases or conditions and/or diseases or conditions associated with senescent cells include pulmonary fibrosis (e.g., idiopathic pulmonary fibrosis, systemic fibrosis-associated lung disease, radiation-associated pulmonary fibrosis), radiation-associated skin fibrosis, liver fibrosis, primary sclerosing cholangitis, diabetic macular edema, age-related macular degeneration, diabetic retinopathy, geographic atrophy, ischemia and reperfusion injury, heart failure, recovery from acute myocardial infarction, pulmonary hypertension, inflammatory bowel disease, colitis, Crohn's disease, diabetes, aging skin (including photoaging-related pigmentation), donor organ transplant survival and function, stem cell transplant survival and function, osteoarthritis, recovery from spinal cord injury, Alzheimer's disease, tau-opathies, progressive supranuclear palsy, and age-related neurological decline (e.g., related to impaired neurovascular coupling). See, e.g., Zhu, Y. I., et al. Aging Cell 14.4 (2015): 644-658; Zhu, Y. I., et al. Aging Cell 15.3 (2016): 428-435; Chang, Jianhui, et al. Nature Medicine 22.1 (2016): 78-83; Zhu, Yi, et al. Aging (Albany NY) 9.3 (2017): 955; Lagares, David, et al. Science Translational Medicine 9.420 (2017): eaa13765; Pan, Jin, et al. International Journal of Radiation Oncology Biology Physics 99.2 (2017): 353-361; Bussian, Tyler J., et al. Nature 562.7728 (2018): 578-582; Moncsek, Anja, et al. Hepatology 67.1 (2018): 247-259; van Willigenburg, Hester, Peter L J de Keizer, and Ron W F de Bruin. Pharmacological Research 130 (2018): 322-330; Walaszczyk, Anna, et al. Aging Cell 18.3 (2019): e12945; Aguayo-Mazzucato, Cristina, et al. Cell Metabolism 30.1 (2019): 129-142; Sessions, Garrett A., et al. The FASEB Journal 33.11 (2019): 12364; Gerdes, Erin O. Wissler, et al. International Review of Neurobiology 155 (2020): 203-234; Sasaki, Motoko, Yasunori Sato, and Yasuni Nakanuma. Journal of Autoimmunity 107 (2020): 102377; Yabluchanskiy, Andriy, et al. Geroscience 42 (2020): 409-428; Dookun, Emily, et al. Aging Cell 19.10 (2020): e13249; Jia, Kangni, et al. Journal of Cardiovascular Pharmacology 764 (2020): 452-460; Sierra-Ramirez, Arantzazu, et al. Aging (Albany NY) 12.12 (2020): 11337; Yang, Hao, et al. Aging (Albany NY) 12.13 (2020): 12750; Lawrie, Allan, and Sheila E. Francis. The Journal of Clinical Investigation 131.11 (2021): e149721; Paramos-de-Carvalho, Diogo, et al. Cell Reports 36.1 (2021): 109334; Tarantini, Stefano, et al. GeroScience 43.5 (2021): 2427-2440; Park, Ji Hee, et al. The British Journal of Dermalology (2021); Fielder, Edward, et al. ELife 11 (2022): e75492; Suzuki, Keiji, et al. Mutation Research Genetic Toxicology and Environmental Mutagenesis 876 (2022): 503448; He, An, et al. American Journal of Transplantation 22.11 (2022): 2529-2547; Johnson, Laura A., et al. Inflammatory Bowel Diseases 28.2 (2022): 161-175; Miura, Yugo, et al. Stem Cell Research & Therapy 13.1 (2022): 222; Cooley, Joseph C., et al. JCI Insight 8.3 (2023): e163762. Watanabe, Yusuke, et al. Hepatology Research 53 (2023): 460-472; and Takaya, Kento, et al., Rejuvenation Research 26.1 (2023): 9-20.

Provided herein is use of a compound of Formula (I) or (II), or a pharmaceutically acceptable salt thereof, or a pharmaceutical composition thereof, for the treatment of a fibrotic disease or condition and/or a disease or condition associated with senescent cells, for example, any of the fibrotic diseases or conditions and/or diseases or conditions associated with senescent cells provided herein.

Provided herein is use of a compound of Formula (I) or (II), or a pharmaceutically acceptable salt thereof, or a pharmaceutical composition thereof, as a medicament for the treatment of a fibrotic disease or condition and/or a disease or condition associated with senescent cells, for example, any of the fibrotic diseases or conditions and/or diseases or conditions associated with senescent cells provided herein.

Provided herein is use of a compound of Formula (I) or (II), or a pharmaceutically acceptable salt thereof, in the manufacture of a medicament for the treatment of a fibrotic disease or condition and/or a disease or condition associated with senescent cells, for example, any of the fibrotic diseases or conditions and/or diseases or conditions associated with senescent cells provided herein.

Also provided herein is a compound of Formula (I) or (II), or a pharmaceutically acceptable salt thereof, or a pharmaceutical composition thereof, for use as a medicament for the treatment of a fibrotic disease or condition and/or a disease or condition associated with senescent cells, for example, any of the fibrotic diseases or conditions and/or diseases or conditions associated with senescent cells provided herein. Provided herein is a compound of Formula (I) or (II), or a pharmaceutically acceptable salt thereof, or a pharmaceutical composition thereof, for use in treating a fibrotic disease or condition and/or a disease or condition associated with senescent cells, for example, any of the fibrotic diseases or conditions and/or diseases or conditions associated with senescent cells provided herein.

Also provided is a method for modulating (e.g., decreasing) BCL-X_{L} protein activity in a cell, comprising contacting the cell with an effective compound of Formula (I) or (II), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I) or (II), or a pharmaceutically acceptable salt thereof, to a subject. In some embodiments, the cell is a cancer cell. In some embodiments, the cell is a mammalian cell. In some embodiments, the cell is a mammalian cancer cell. In some embodiments, the cancer cell is any cancer as described herein.

As used herein, the term “contacting” refers to the bringing together of indicated moieties in an in vitro system or an in vivo system. For example, “contacting” a cell with a compound provided herein includes the administration of a compound provided herein to the cell, in vitro or in vivo, including, for example, introducing a compound provided herein into a sample containing cells (e.g., grown in culture or derived from a patient), an organoid, or an organism (e.g., an animal (e.g., an animal bearing a tumor), or a human).

Also provided is a method of modulating (e.g., decreasing) the level of BCL-X_{L} protein in a cell, comprising contacting the cell with a compound of Formula (I) or (II), or a pharmaceutically acceptable salt thereof. In some embodiments, the level of BCL-X_{L} protein is decreased by at least 30% (e.g., at least 40%, at least 50%, at least 60%, at least 70%, at least 80%, at least 90%, at least 95%, at least 97%, or at least 99%) compared to a cell not contacted with the compound of Formula (I) or (II), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I) or (II), or a pharmaceutically acceptable salt thereof, to a subject having a cell having a BCL-X_{L} protein. In some embodiments, the cell is a cancer cell. In some embodiments, the cell is a mammalian cell. In some embodiments, the cell is a mammalian cancer cell. In some embodiments, the cancer cell is any cancer as described herein.

Also provided is a method of inducing ubiquitination of a BCL-X_{L} protein in a cell, comprising contacting the cell with a compound of Formula (I) or (II), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I) or (II), or a pharmaceutically acceptable salt thereof, to a subject having a cell having a BCL-X_{L} protein. In some embodiments, the cell is a cancer cell. In some embodiments, the cell is a mammalian cell. In some embodiments, the cell is a mammalian cancer cell. In some embodiments, the cancer cell is any cancer as described herein.

Also provided is a method of forming a ternary complex comprising a BCL-X_{L} protein, a compound of Formula (I) or (II), or a pharmaceutically acceptable salt thereof, and a VHL protein or fragment thereof in a cell, comprising contacting the cell with a compound of Formula (I) or (II), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I) or (II), or a pharmaceutically acceptable salt thereof, to a subject having a cell having a BCL-X_{L} protein. In some embodiments, the cell is a cancer cell. In some embodiments, the cell is a mammalian cell. In some embodiments, the cell is a mammalian cancer cell. In some embodiments, the cancer cell is any cancer as described herein.

Also provided herein is a method of inhibiting cell proliferation, in vitro or in vivo, the method comprising contacting a cell with an effective amount of a compound of Formula (I) or (II), or a pharmaceutically acceptable salt thereof, or a pharmaceutical composition thereof as defined herein.

Further provided herein is a method of increasing cell death, in vitro or in vivo, the method comprising contacting a cell with an effective amount of a compound of Formula (I) or (II), or a pharmaceutically acceptable salt thereof, or a pharmaceutical composition thereof as defined herein. Also provided herein is a method of increasing tumor cell death in a subject. The method comprises administering to the subject a compound of Formula (I) or (II), or a pharmaceutically acceptable salt thereof, in an amount effective to increase tumor cell death.

When employed as pharmaceuticals, the compounds of Formula (I) or (II), or pharmaceutically acceptable salts thereof, can be administered in the form of pharmaceutical compositions as described herein.

Also provided herein is a method for inducing degradation of a BCL-X_{L} protein in a mammalian cell, the method comprising contacting the mammalian cell with an effective amount of a compound of Formula (I) or (II), or a pharmaceutically acceptable salt thereof.

Also provided herein is a method of treating a subject having a cancer, wherein the method comprises:
administering a therapeutically effective amount of a compound of Formula (I) or (II), or a pharmaceutically acceptable salt thereof, as a monotherapy or in conjunction with a first anticancer agent to the subject who has been administered one or more doses of the first anticancer agent to the subject for a period of time.

Also provided herein is a method of treating a subject having a cancer, wherein the method comprises:
(a) administering one or more doses of a first anticancer agent to the subject for a period of time; and
(b) after (a), administering a therapeutically effective amount of a compound of Formula (I) or (II), or a pharmaceutically acceptable salt thereof, as a monotherapy or in conjunction with the first anticancer agent to the subject.

Also provided herein is a method of treating a subject having a cancer, wherein the method comprises:
(a) administering one or more doses of a first anticancer agent to the subject for a period of time; and
(b) after (a), administering a therapeutically effective amount of a compound of Formula (I) or (II), or a pharmaceutically acceptable salt thereof, as a monotherapy or in conjunction with a second anticancer agent to the subject.

### Combinations

In any of the indications described herein, a compound of Formula (I) or (II), or a pharmaceutically acceptable salt thereof, can be used as a monotherapy. In some embodiments, a compound of Formula (I) or (II), or a pharmaceutically acceptable salt thereof, can be used prior to administration of an additional therapeutic agent or additional therapy. For example, a subject in need thereof can be administered one or more doses of a compound of Formula (I) or (II), or a pharmaceutically acceptable salt thereof, for a period of time and then undergo at least partial resection of the tumor. In some embodiments, the treatment with one or more doses of a compound of Formula (I) or (II), or a pharmaceutically acceptable salt thereof, reduces the size of the tumor (e.g., the tumor burden) prior to the at least partial resection of the tumor.

In some embodiments, a subject in need thereof can be administered one or more doses of a compound of Formula (I) or (II), or a pharmaceutically acceptable salt thereof, for a period of time and under one or more rounds of radiation therapy. In some embodiments, the treatment with one or more doses of a compound of Formula (I) or (II), or a pharmaceutically acceptable salt thereof, reduces the size of the tumor (e.g., the tumor burden) prior to the one or more rounds of radiation therapy.

In some embodiments of any the methods described herein, the compound of Formula (I) or (II), or a pharmaceutically acceptable salt thereof, is administered in combination with a therapeutically effective amount of at least one additional therapeutic agent selected from one or more additional therapies or therapeutic (e.g., chemotherapeutic) agents.

Non-limiting examples of additional therapies and therapeutic agents include: RAS pathway targeted therapeutic agents (e.g., Ras/RAF/MEK/PI3K pathway inhibitors, (e.g., Ras inhibitors (e.g., KRas inhibitors), KRas-targeted therapeutic agents, SOS1 inhibitors, SOS1/Ras protein-protein interaction inhibitors, SHP2 inhibitors, PI3K-AKT-mTOR pathway inhibitors)), kinase-targeted therapeutics (e.g., MEK inhibitors, ERK inhibitors, Raf inhibitors (e.g., BRaf inhibitors), PI3K inhibitors, Abl inhibitors (e.g., BCR-Abl inhibitors), ALK inhibitors, AKT inhibitors, AURKA inhibitors, mTOR inhibitors, CDK2 inhibitors, CDK4/5 inhibitors, CDK4/6 inhibitors, CDK7 inhibitors, CDK9 inhibitors, MET (also known as cMET) inhibitors, FAK inhibitors, FGFR1 inhibitors, FGFR2 inhibitors, FGFR3 inhibitors, FGFR4 inhibitors, ErbB family inhibitors (e.g., EGFR inhibitors, anti-EGFR antibodies or anti-EGFR antibody-drug conjugates, HER2 inhibitors, anti-HER2 antibodies or anti-HER2 antibody-drug conjugates), JAK inhibitors (e.g., JAK inhibitors having activity against JAK1, JAK2, and/or JAK1/2), Src inhibitors, VEGFR inhibitors), LSD1 inhibitors, EZH2 inhibitors, BET inhibitors, STING agonists, telomerase inhibitors, mTORC1 inhibitors, YAP inhibitors, proteasome inhibitors, farnesyl transferase inhibitors, Hif2a inhibitors, HSP90 inhibitors, PTEN inhibitors, PARP inhibitors, signal transduction pathway inhibitors, checkpoint inhibitors, modulators of the apoptosis pathway (e.g., BCL-2 inhibitors), chemotherapeutics, angiogenesis-targeted therapies, immune-targeted agents, including immunomodulatory imide drugs (sometimes called “IMiDs” or “CELMoDs”) and immunotherapy (e.g., anti-PD1 therapy or anti-PD-L1 therapy), and radiotherapy.

As used herein, a biosimilar antibody refers to an antibody or antigen-binding fragment that has the same primary amino acid sequence as compared to a reference antibody and optionally, may have detectable differences in post-translation modifications (e.g., glycosylation and/or phosphorylation) as compared to the reference antibody (e.g., a different glycoform).

In some embodiments, the additional therapy or therapeutic agent is an Abl inhibitor (e.g., a BCR-Abl inhibitor), an ALK inhibitor, an AURKA inhibitor, a BCL-2 inhibitor, a Braf inhibitor, a CDK2 inhibitor, a CDK4/6 inhibitor, a CDK7 inhibitor, a CDK9 inhibitor, an EGFR inhibitor, an anti-EGFR antibody or anti-EGFR antibody-drug conjugate, an ERK inhibitor, an EZH2 inhibitor, a FGFR1 inhibitor, a FGFR2 inhibitor, a FGFR3 inhibitor, a FGFR4 inhibitor, a HER2 inhibitor, an anti-HER2 antibody or anti-HER2 antibody-drug conjugate, a JAK inhibitor, a KRas inhibitor, a MEK inhibitor, a MET inhibitor, a Hif2a inhibitor, a PARP inhibitor, a VEGFR inhibitor, an LSD1 inhibitor, a BET inhibitor, a STING agonist, a telomerase inhibitor, a TORC1/2 inhibitor, an immunomodulatory imide drug, immunotherapy (e.g., a PD-1 inhibitor (e.g., anti-PD1 therapy), a PD-L1 inhibitor (e.g., anti-PD-L1 therapy)), chemotherapy, radiotherapy, or a combination thereof.

In some embodiments, the additional therapy or therapeutic agent is an Abl degrader (e.g., a BCR-Abl degrader), an ALK degrader, an AURKA degrader, a BCL-2 degrader, a BRaf degrader, a CDK2 degrader, a CDK4/6 degrader, a CDK7 degrader, a CDK9 degrader, an EGFR degrader, an ERK degrader, an EZH2 degrader, a FGFR1 degrader, a FGFR2 degrader, a FGFR3 degrader, a FGFR4 degrader, a HER2 degrader, a JAK2 degrader, a KRas degrader, a MEK degrader, a MET degrader, a Hif2a degrader, a PARP degrader, a VEGFR degrader, an LSD1 degrader, a BET degrader, a telomerase degrader, a TORC1/2 degrader, an immunomodulatory imide drug, immunotherapy (e.g., anti-PD1 therapy or anti-PD-L1 therapy), chemotherapy, radiotherapy, or a combination thereof.

In some embodiments, the Abl inhibitor (e.g., BCR-Abl inhibitor) is asciminib (e.g., asciminib hydrochloride), bafetinib, bosutinib (e.g., bosutinib monohydrate), danusertib, dasatinib (e.g., dasatinib monohydrate), flumatinib (e.g., flumatinib mesylate), imatinib (e.g., imatinib mesylate), nilotinib (e.g., nilotinib monochloride monohydrate), olverembatinib (e.g., olverembatinib mesylate), ponatinib (e.g., ponatinib hydrochloride), radotinib (e.g., radotinib dihydrochloride), ruserontinib, vandetanib, AN-019, AT-9283, IkT-148009, NPB-001-056, or a combination thereof.

In some embodiments, the ALK inhibitor is alectinib (e.g., alectinib hydrochloride), brigatinib, ceritinib, crizotinib, ensartinib (e.g., ensartinib hydrochloride), entrectinib, fidrisertib, lorlatinib, TQ-B-3101, TQ-B-3139, or a combination thereof. In some embodiments, the ALK inhibitor is alectinib (e.g., alectinib hydrochloride), brigatinib, ceritinib, crizotinib, ensartinib (e.g., ensartinib hydrochloride), fidrisertib, lorlatinib, TQ-B-3101, TQ-B-3139, or a combination thereof.

In some embodiments, the AURKA inhibitor is alisertib, danusertib, ilorasertib, tinengotinib, AT-9283, BI-811283, ENMD-2076, or a combination thereof.

In some embodiments, the BCL-2 inhibitor is lisaftoclax, navitoclax, obatoclax, venetoclax, oblimersen (e.g., oblimersen sodium), beclanorsen, AZD-0466, BGB-11417, UBX-1325 (or a phosphate prodrug thereof), UBX-1967 (or a phosphate prodrug thereof), ZN-d5, or a combination thereof.

In some embodiments, the cancer is a lung cancer (e.g., SCLC), and the additional therapy or therapeutic agent is a BCL-2 inhibitor (e.g., lisaftoclax, navitoclax, obatoclax, venetoclax, oblimersen (e.g., oblimersen sodium), beclanorsen, AZD-0466, BGB-11417, UBX-1325 (or a phosphate prodrug thereof), UBX-1967 (or a phosphate prodrug thereof), or ZN-d5). In some embodiments, the cancer is a lung cancer (e.g., SCLC), and the additional therapy or therapeutic agent is venetoclax.

In some embodiments, the cancer is a non-Hodgkin lymphoma, and the additional therapy or therapeutic agent is a BCL-2 inhibitor (e.g., lisaftoclax, navitoclax, obatoclax, venetoclax, oblimersen (e.g., oblimersen sodium), beclanorsen, AZD-0466, BGB-11417, UBX-1325 (or a phosphate prodrug thereof), UBX-1967 (or a phosphate prodrug thereof), or ZN-d5). In some embodiments, the cancer is a non-Hodgkin lymphoma, and the additional therapy or therapeutic agent is venetoclax.

In some embodiments, the BRaf inhibitor is avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, PLX-4720, or a combination thereof.

In some embodiments, a BRaf V600E mutation can be detected in a sample from the subject (e.g., detecting a BRAF gene having a mutation corresponding to a V600E mutation in BRaf protein and/or detecting a BRaf protein having a V600E mutation). In some embodiments, the subject was determined (e.g., prior to administration of a compound of Formula (I) or (II)) to have a cancer having a BRaf V600E mutation.

In some embodiments, the cancer is a BRaf mutant CRC (e.g., BRaf V600E mutant CRC), and the additional therapy or therapeutic agent is a BRaf inhibitor (e.g., avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720). In some embodiments, the cancer is a BRaf mutant CRC (e.g., BRaf V600E mutant CRC), and the additional therapy or therapeutic agent is a BRaf inhibitor (e.g., avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720) and an anti-EGFR antibody or anti-EGFR antibody-drug conjugate (e.g., amivantamab (e.g., amivantamab-vmjw, or a biosimilar thereof), cetuximab (e.g., ERBITUX® (cetuximab), or a biosimilar thereof (e.g., CMAB-009, CPGJ-602, or KL-140)), cetuximab sarotalocan (AKALUX® (cetuximab sarotalocan), or a biosimilar thereof), depatuxizumab, duligotuzumab, futuximab, imgatuzumab, modotuximab, necitumumab (e.g., PORTRAZZA® (necitumumab), or a biosimilar thereof), nimotuzumab (e.g., BIOMAb EGFR® (nimotuzumab), or a biosimilar thereof), panitumumab (e.g., VECTIBIX® (panitumumab), or a biosimilar thereof), tomuzotuximab, zalutumumab, EMD-55900, EMD-82633, GC-1118, HLX-07, ICR-62, SCT-200, SI—B-001, TAS-0313, or biosimilars thereof). In some embodiments, the cancer is a BRaf mutant CRC (e.g., BRaf V600E mutant CRC), and the additional therapy or therapeutic agent is a BRaf inhibitor (e.g., avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720) and cetuximab (e.g., ERBITUX® (cetuximab), or a biosimilar thereof (e.g., CMAB-009, CPGJ-602, or KL-140)).

In some embodiments, the cancer is a BRaf mutant CRC (e.g., BRaf V600E mutant CRC), and the additional therapy or therapeutic agent is a BRaf inhibitor (e.g., avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720) and panitumumab (e.g., VECTIBIX® (panitumumab), or a biosimilar thereof). In some embodiments, the cancer is a BRaf mutant CRC (e.g., BRaf V600E mutant CRC), and the additional therapy or therapeutic agent is a dabrafenib (e.g., dabrafenib mesylate, GSK2118436), encorafenib (e.g., BRAFTOVI™ LGX818), or vemurafenib (e.g., ZELBORAF®, RO5185426) and cetuximab (e.g., ERBITUX® (cetuximab), or a biosimilar thereof (e.g., CMAB-009, CPGJ-602, or KL-140)). In some embodiments, the cancer is a BRaf mutant CRC (e.g., BRaf V600E mutant CRC), and the additional therapy or therapeutic agent is dabrafenib (e.g., dabrafenib mesylate, GSK2118436), encorafenib (e.g., BRAFTOVI™, LGX818), or vemurafenib (e.g., ZELBORAF®, RO5185426) and panitumumab (e.g., VECTIBIX® (panitumumab), or a biosimilar thereof). In some embodiments, the cancer is a BRaf mutant CRC (e.g., BRaf V600E mutant CRC), and the additional therapy or therapeutic agent is a dabrafenib (e.g., dabrafenib mesylate, GSK2118436) and cetuximab (e.g., ERBITUX® (cetuximab), or a biosimilar thereof (e.g., CMAB-009, CPGJ-602, or KL-140)). In some embodiments, the cancer is a BRaf mutant CRC (e.g., BRaf V600E mutant CRC), and the additional therapy or therapeutic agent is dabrafenib (e.g., dabrafenib mesylate, GSK2118436) and panitumumab (e.g., VECTIBIX® (panitumumab), or a biosimilar thereof).

In some embodiments, the cancer is a BRaf mutant NSCLC (e.g., BRaf V600E mutant NSCLC), and the additional therapy or therapeutic agent is a BRaf inhibitor (e.g., avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720) and a MEK inhibitor (e.g., avutometinib, binimetinib, cobimetinib (e.g., cobimetinib fumarate), mirdametinib, pimasertib, refametinib, selumetinib (e.g., selumetinib sulfate), trametinib (e.g., trametinib dimethyl sulfoxide), zapnometinib, FCN-159, GSK-1120212, NFX-179, or TAK-733). In some embodiments, the cancer is a BRaf mutant NSCLC (e.g., BRaf V600E mutant NSCLC), and the additional therapy or therapeutic agent is dabrafenib (e.g., dabrafenib mesylate, GSK2118436), encorafenib (e.g., BRAFTOVI™, LGX818), or vemurafenib (e.g., ZELBORAF®, RO5185426), and binimetinib, cobimetinib (e.g., cobimetinib fumarate), selumetinib (e.g., selumetinib sulfate), or trametinib (e.g., trametinib dimethyl sulfoxide). In some embodiments, the cancer is a BRaf mutant NSCLC (e.g., BRaf V600E mutant NSCLC), and the additional therapy or therapeutic agent is dabrafenib (e.g., dabrafenib mesylate, GSK2118436) and trametinib (e.g., trametinib dimethyl sulfoxide). In some embodiments, the cancer is a BRaf mutant NSCLC (e.g., BRaf V600E mutant NSCLC), and the additional therapy or therapeutic agent is vemurafenib (e.g., ZELBORAF®, RO5185426), and cobimetinib (e.g., cobimetinib fumarate). In some embodiments, the cancer is a BRaf mutant NSCLC (e.g., BRaf V600E mutant NSCLC), and the additional therapy or therapeutic agent is encorafenib (e.g., BRAFTOVI™, LGX818) and binimetinib.

In some embodiments, the cancer is a BRaf mutant melanoma (e.g., BRaf V600E mutant melanoma), and the additional therapy or therapeutic agent is a BRaf inhibitor (e.g., avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720). In some embodiments, the cancer is a BRaf mutant melanoma (e.g., BRaf V600E mutant melanoma), and the additional therapy or therapeutic agent is a BRaf inhibitor (e.g., avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL₂₈₁), C₁₇₀₇₁₄₇₉—F, CHIR-265 (RAF265), FORE-8394 (PLX-8394), GDC-0879, GDC-5573 (HM95573), HLX-208, PLX-3603, or PLX-4720) and a MEK inhibitor (e.g., avutometinib, binimetinib, cobimetinib (e.g., cobimetinib fumarate), mirdametinib, pimasertib, refametinib, selumetinib (e.g., selumetinib sulfate), trametinib (e.g., trametinib dimethyl sulfoxide), zapnometinib, FCN-159, GSK-1120212, NFX-179, or TAK-733). In some embodiments, the cancer is a BRaf mutant melanoma (e.g., BRaf V600E mutant melanoma), and the additional therapy or therapeutic agent is dabrafenib (e.g., dabrafenib mesylate, GSK2118436), encorafenib (e.g., BRAFTOVI™, LGX818), or vemurafenib (e.g., ZELBORAF®, RO5185426), and binimetinib, cobimetinib (e.g., cobimetinib fumarate), selumetinib (e.g., selumetinib sulfate), or trametinib (e.g., trametinib dimethyl sulfoxide). In some embodiments, the cancer is a BRaf mutant melanoma (e.g., BRaf V600E mutant melanoma), and the additional therapy or therapeutic agent is dabrafenib (e.g., dabrafenib mesylate, GSK2118436) and trametinib (e.g., trametinib dimethyl sulfoxide). In some embodiments, the cancer is a BRaf mutant melanoma (e.g., BRaf V600E mutant melanoma), and the additional therapy or therapeutic agent is vemurafenib (e.g., ZELBORAF®, RO5185426), and cobimetinib (e.g., cobimetinib fumarate). In some embodiments, the cancer is a BRaf mutant melanoma (e.g., BRaf V600E mutant melanoma), and the additional therapy or therapeutic agent is encorafenib (e.g., BRAFTOVI™ LGX818) and binimetinib. In some such embodiments, the subject has previously been treated with an immunotherapy.

In some embodiments, the CDK2 inhibitor is ebvaciclib, fadraciclib, milciclib, pacritinib (e.g., pacritinib citrate), roniciclib, roscovitine, BLU-222, NUV-422, PF-07104091, TQB-3616, or a combination thereof.

In some embodiments, the CDK4/6 inhibitor is abemaciclib, birociclib, dalpiciclib, lerociclib, milciclib, palbociclib, ribociclib (e.g., ribociclib succinate), riviciclib, roniciclib, trilaciclib (e.g., trilaciclib dihydrochloride), FCN-437, TQB-3616, or a combination thereof.

In some embodiments, the CDK7 inhibitor is milciclib, roscovitine, samuraciclib, or a combination thereof.

In some embodiments, the CDK9 inhibitor is fadraciclib, riviciclib, roniciclib, roscovitine, zotiraciclib, AZD-4573, KB-0742, or a combination thereof.

In some embodiments, the EGFR inhibitor is abivertinib, afatinib (e.g., afatinib dimaleate), alflutinib (e.g., alflutinib mesylate), almonertinib (e.g., almonertinib mesylate), befotertinib, brigatinib, canertinib, dacomitinib (e.g., dacomitinib monohydrate), dovitinib, erlotinib (e.g., erlotinib hydrochloride), gefitinib, icotinib, lapatinib (e.g., lapatinib ditosylate monohydrate), larotinib, lazertinib, limertinib, mobocertinib (e.g., mobocertinib succinate), nazartinib, neratinib (e.g., neratinib maleate), olmutinib, osimertinib (e.g., osimertinib mesylate), pelitinib, poziotinib, pyrotinib (e.g., pyrotinib maleate), ruserontinib (SKLB-1028), sapitinib, sunvozertinib, tesevatinib, vandetanib, varlitinib, zorifertinib, BIBW-2948, BPI-7711, HA-121-28, SH-1028, an anti-EGFR antibody or anti-EGFR antibody-drug conjugate, or a combination thereof.

In some embodiments, the anti-EGFR antibody or anti-EGFR antibody-drug conjugate is amivantamab (e.g., amivantamab-vmjw, or a biosimilar thereof), cetuximab (e.g., ERBITUX® (cetuximab), or a biosimilar thereof (e.g., CMAB-009, CPGJ-602, or KL-140)), cetuximab sarotalocan (AKALUX® (cetuximab sarotalocan), or a biosimilar thereof), depatuxizumab, duligotuzumab, futuximab, imgatuzumab, modotuximab, necitumumab (e.g., PORTRAZZA® (necitumumab), or a biosimilar thereof), nimotuzumab (e.g., BIOMAb EGFR® (nimotuzumab), or a biosimilar thereof), panitumumab (e.g., VECTIBIX® (panitumumab), or a biosimilar thereof), tomuzotuximab, zalutumumab, EMD-55900, EMD-82633, GC-1118, HLX-07, ICR-62, SCT-200, SI—B-001, TAS-0313, biosimilars thereof, or a combination thereof. In some embodiments, the anti-EGFR antibody or anti-EGFR antibody-drug conjugate is amivantamab (e.g., amivantamab-vmjw, or a biosimilar thereof), cetuximab (e.g., ERBITUX® (cetuximab), or a biosimilar thereof (e.g., CMAB-009, CPGJ-602, or KL-140)), cetuximab sarotalocan (AKALUX® (cetuximab sarotalocan), or a biosimilar thereof), depatuxizumab, duligotuzumab, futuximab, imgatuzumab, modotuximab, necitumumab (e.g., PORTRAZZA® (necitumumab), or a biosimilar thereof), nimotuzumab (e.g., BIOMAb EGFR® (nimotuzumab), or a biosimilar thereof), panitumumab (e.g., VECTIBIX® (panitumumab), or a biosimilar thereof), tomuzotuximab, zalutumumab, EMD-55900, EMD-82633, GC-1118, HLX-07, ICR-62, SCT-200, SI—B-001, biosimilars thereof, or a combination thereof.

In some embodiments, an EGFR mutation (e.g., an EGFR exon 19 deletion or an EGFR L858R mutation (with or without an EGFR T790M mutation)) can be detected in a sample from the subject (e.g., detecting an EGFR gene having a mutation (e.g., a mutation corresponding to an EGFR exon 19 deletion or an EGFR L858R mutation (with or without an EGFR T790M mutation) in an EGFR protein) and/or detecting an EGFR protein having a mutation (e.g., an EGFR exon 19 deletion or an EGFR L858R mutation (with or without an EGFR T790M mutation))). In some embodiments, the subject was determined (e.g., prior to administration of a compound of Formula (I) or (II)) to have a cancer having an EGFR mutation (e.g., an EGFR exon 19 deletion or an EGFR L858R mutation (with or without an EGFR T790M mutation)).

In some embodiments, the cancer is an EGFR mutant NSCLC (e.g., EGFR exon 19 deletion NSCLC or EGFR L858R (with or without T790M) mutant NCLC), and the additional therapy or therapeutic agent is an EGFR inhibitor (e.g., abivertinib, afatinib (e.g., afatinib dimaleate), alflutinib (e.g., alflutinib mesylate), almonertinib (e.g., almonertinib mesylate), befotertinib, brigatinib, canertinib, dacomitinib (e.g., dacomitinib monohydrate), dovitinib, erlotinib (e.g., erlotinib hydrochloride), gefitinib, icotinib, lapatinib (e.g., lapatinib ditosylate monohydrate), larotinib, lazertinib, limertinib, mobocertinib (e.g., mobocertinib succinate), nazartinib, neratinib (e.g., neratinib maleate), olmutinib, osimertinib (e.g., osimertinib mesylate), pelitinib, poziotinib, pyrotinib (e.g., pyrotinib maleate), ruserontinib (SKLB-1028), sapitinib, sunvozertinib, tesevatinib, vandetanib, varlitinib, zorifertinib, BIBW-2948, BPI-7711, HA-121-28, SH-1028, or an anti-EGFR antibody or anti-EGFR antibody-drug conjugate). In some embodiments, the cancer is an EGFR mutant NSCLC (e.g., EGFR exon 19 deletion NSCLC or EGFR L858R (with or without T790M) mutant NCLC), and the additional therapy or therapeutic agent is osimertinib (e.g., osimertinib mesylate).

In some embodiments, the ERK inhibitor is rineterkib, temuterkib, ulixertinib, ASN-0007, ASTX-029, ATG-017, BPI-27336, HH-2710, JSI-1187, MK-8353, or a combination thereof.

In some embodiments, the EZH2 inhibitor is lirametostat, tazemetostat (e.g., tazemetostat hydrobromide), valemetostat (e.g., valemetostat tosylate), tulmimetostat (CPI-0209), EBI-2511, HH-2853, HM-97662, PF-6821497, SHR-2554, XNW-5004, or a combination thereof. In some embodiments, the EZH2 inhibitor also inhibits EZH1 (also referred to as an EZH1/2 inhibitor).

In some embodiments, the cancer is peripheral T cell lymphoma, and the additional therapy or therapeutic agent is an EZH2 inhibitor (e.g., lirametostat, tazemetostat (e.g., tazemetostat hydrobromide), valemetostat (e.g., valemetostat tosylate), tulmimetostat (CPI-0209), EBI-2511, HH-2853, HM-97662, PF-6821497, SHR-2554, or XNW-5004).

In some embodiments, the FGFR1 inhibitor is danusertib, dovitinib, erdafitinib, futibatinib, infigratinib (e.g., infigratinib phosphate), lenvatinib (e.g., lenvatinib mesylate), lucitanib, nintedanib (e.g., nintedanib esylate), pemigatinib, surufatinib, tasurgratinib, tinengotinib, zoligratinib, FH-2001, HMPL-453, LY-2874455, or a combination thereof.

In some embodiments, the FGFR2 inhibitor is erdafitinib, futibatinib, infigratinib (e.g., infigratinib phosphate), lucitanib, pemigatinib, tasurgratinib, zoligratinib, bemarituzumab (or biosimilars thereof), FH-2001, HMPL-453, LY-2874455, or a combination thereof.

In some embodiments, the FGFR3 inhibitor is dovitinib, erdafitinib, futibatinib, infigratinib (e.g., infigratinib phosphate), lucitanib, masitinib, nintedanib, pemigatinib, tasurgratinib, zoligratinib, vofatamab (or biosimilars thereof), EXEL-0999, FH-2001, HMPL-453, LY-2874455, or a combination thereof.

In some embodiments, the FGFR4 inhibitor is axitinib, erdafitinib, futibatinib, infigratinib (e.g., infigratinib phosphate), irpagratinib, nintedanib, pemigatinib, FH-2001, H3B-6527, LY-2874455, or a combination thereof.

In some embodiments, the HER2 inhibitor is afatinib (e.g., afatinib dimaleate), dacomitinib (e.g., dacomitinib monohydrate), lapatinib (e.g., lapatinib ditosylate monohydrate), mobocertinib (e.g., mobocertinib succinate), neratinib (e.g., neratinib maleate), poziotinib, pyrotinib (e.g., pyrotinib maleate), sunvozertinib, tesevatinib, tucatinib, varlitinib, an anti-HER2 antibody or anti-HER2 antibody-drug conjugate, or a combination thereof.

In some embodiments, the anti-HER2 antibody or anti-HER2 antibody-drug conjugate is anbenitamab, cinrebafusp alfa, coprelotamab, disitamab vedotin, ertumaxomab, gancotamab, inetetamab, margetuximab (e.g., margetuximab-cmkb, or a biosimilar thereof), pertuzumab (e.g., PERJETA® (pertuzumab), or a biosimilar thereof (e.g., HLX-11)), trastuzumab (e.g., HERCEPTIN® (trastuzumab), or a biosimilar thereof (e.g., FACEPTOR® (trastuzumab), HERTICAD® (trastuzumab), TUZNUE® (trastuzumab), ZERCEPAC® (trastuzumab), trastuzumab-anns, trastuzumab-dkst, trastuzumab-dttb, trastuzumab-pkrb, trastuzumab-qyyp, EG-12014, or TX-05)), trastuzumab deruxtecan (e.g., fam-trastuzumab deruxtecan-nxki, or a biosimilar thereof), trastuzumab duocarmazine, trastuzumab emtansine (e.g., KADCYLA® (trastuzumab emtansine), or a biosimilar thereof (e.g., UJVIRA® (trastuzumab emtansine))), trastuzumab hyaluronidase (e.g., trastuzumab hyaluronidase-oysk, or a biosimilar thereof), zanidatamab, zenocutuzumab, AVX-901, IDM-1, TPIV-100, TAA-013, SHR-A1811, BAT-8001, MDX-210, Alpha-Her2-pAF1-AS-269, MRG-002, DF-1001, AC-101, MM-111, biosimilars thereof, or a combination thereof. In some embodiments, the anti-HER2 antibody or anti-HER2 antibody-drug conjugate is anbenitamab, cinrebafusp alfa, coprelotamab, disitamab vedotin, ertumaxomab, gancotamab, inetetamab, margetuximab (e.g., margetuximab-cmkb, or a biosimilar thereof), pertuzumab (e.g., PERJETA® (pertuzumab), or a biosimilar thereof (e.g., HLX-11)), trastuzumab (e.g., HERCEPTIN® (trastuzumab), or a biosimilar thereof (e.g., FACEPTOR® (trastuzumab), HERTICAD® (trastuzumab), TUZNUE® (trastuzumab), ZERCEPAC® (trastuzumab), trastuzumab-anns, trastuzumab-dkst, trastuzumab-dttb, trastuzumab-pkrb, trastuzumab-qyyp, EG-12014, or TX-05)), trastuzumab deruxtecan (e.g., fam-trastuzumab deruxtecan-nxki, or a biosimilar thereof), trastuzumab duocarmazine, trastuzumab emtansine (e.g., KADCYLA® (trastuzumab emtansine), or a biosimilar thereof (e.g., UJVIRA® (trastuzumab emtansine))), trastuzumab hyaluronidase (e.g., trastuzumab hyaluronidase-oysk, or a biosimilar thereof), zanidatamab, zenocutuzumab, TAA-013, SHR-A1811, BAT-8001, MDX-210, Alpha-Her2-pAF1-AS-269, MRG-002, DF-1001, AC-101, MM-111, biosimilars thereof, or a combination thereof.

In some embodiments, HER2+ status can be detected in a sample from the subject (e.g., via immunohistochemistry (IHC) and/or fluorescent in situ hybridization (FISH)). In some embodiments, the subject was determined (e.g., prior to administration of a compound of Formula (I) or (II)) to have a cancer with HER2+ status.

In some embodiments, HER2 low status can be detected in a sample from the subject (e.g., via IHC and/or FISH). In some embodiments, the subject was determined (e.g., prior to administration of a compound of Formula (I) or (II)) to have a cancer with HER2 low status.

In some embodiments, HER2− status can be detected in a sample from the subject (e.g., via IHC and/or FISH). In some embodiments, the subject was determined (e.g., prior to administration of a compound of Formula (I) or (II)) to have a cancer with HER2− status.

In some embodiments, ER expression status can be detected in a sample from the subject (e.g., via IHC and/or FISH). In some embodiments, the subject was determined (e.g., prior to administration of a compound of Formula (I) or (II)) to have a cancer with ER expression. In some embodiments, the subject was determined (e.g., prior to administration of a compound of Formula (I) or (II)) to have a cancer without ER expression.

In some embodiments, PR expression status can be detected in a sample from the subject (e.g., via IHC, and/or FISH). In some embodiments, the subject was determined (e.g., prior to administration of a compound of Formula (I) or (II)) to have a cancer with PR expression. In some embodiments, the subject was determined (e.g., prior to administration of a compound of Formula (I) or (II)) to have a cancer without PR expression.

In some embodiments, the cancer is a HER2+ breast cancer (e.g., HER2+ breast cancer with ER expression, HER2+ breast cancer without ER expression), and the additional therapy is a HER2 inhibitor (e.g., afatinib (e.g., afatinib dimaleate), dacomitinib (e.g., dacomitinib monohydrate), lapatinib (e.g., lapatinib ditosylate monohydrate), mobocertinib (e.g., mobocertinib succinate), neratinib (e.g., neratinib maleate), poziotinib, pyrotinib (e.g., pyrotinib maleate), sunvozertinib, tesevatinib, tucatinib, varlitinib, or an anti-HER2 antibody or anti-HER2 antibody-drug conjugate). In some embodiments, the cancer is a HER2+ breast cancer (e.g., HER2+ breast cancer with ER expression, HER2+ breast cancer without ER expression), and the additional therapy is tucatinib. In some embodiments, the cancer is a HER2+ breast cancer (e.g., HER2+ breast cancer with ER expression, HER2+ breast cancer without ER expression), and the additional therapy is an anti-HER2 antibody or anti-HER2 antibody-drug conjugate (e.g., anbenitamab, cinrebafusp alfa, coprelotamab, disitamab vedotin, ertumaxomab, gancotamab, inetetamab, margetuximab (e.g., margetuximab-cmkb, or a biosimilar thereof), pertuzumab (e.g., PERJETA® (pertuzumab), or a biosimilar thereof (e.g., HLX-11)), trastuzumab (e.g., HERCEPTIN® (trastuzumab), or a biosimilar thereof (e.g., FACEPTOR® (trastuzumab), HERTICAD® (trastuzumab), TUZNUE® (trastuzumab), ZERCEPAC® (trastuzumab), trastuzumab-anns, trastuzumab-dkst, trastuzumab-dttb, trastuzumab-pkrb, trastuzumab-qyyp, EG-12014, or TX-05)), trastuzumab deruxtecan (e.g., fam-trastuzumab deruxtecan-nxki, or a biosimilar thereof), trastuzumab duocarmazine, trastuzumab emtansine (e.g., KADCYLA® (trastuzumab emtansine), or a biosimilar thereof (e.g., UJVIRA® (trastuzumab emtansine))), trastuzumab hyaluronidase (e.g., trastuzumab hyaluronidase-oysk, or a biosimilar thereof), zanidatamab, zenocutuzumab, AVX-901, IDM-1, TPIV-100, TAA-013, SHR-A1811, BAT-8001, MDX-210, Alpha-Her2-pAF1-AS-269, MRG-002, DF-1001, AC-101, MM-111, or biosimilars thereof). In some embodiments, the cancer is a HER2+ breast cancer (e.g., HER2+ breast cancer with ER expression, HER2+ breast cancer without ER expression), and the additional therapy is trastuzumab (e.g., HERCEPTIN® (trastuzumab), or a biosimilar thereof (e.g., FACEPTOR® (trastuzumab), HERTICAD® (trastuzumab), TUZNUE® (trastuzumab), ZERCEPAC® (trastuzumab), trastuzumab-anns, trastuzumab-dkst, trastuzumab-dttb, trastuzumab-pkrb, trastuzumab-qyyp, EG-12014, or TX-05)). In some embodiments, the cancer is a HER2+ breast cancer (e.g., HER2+ breast cancer with ER expression, HER2+ breast cancer without ER expression), and the additional therapy is trastuzumab deruxtecan (e.g., fam-trastuzumab deruxtecan-nxki, or a biosimilar thereof). In some embodiments, the cancer is a HER2+ breast cancer (e.g., HER2+ breast cancer with ER expression, HER2+ breast cancer without ER expression), and the additional therapy is trastuzumab emtansine, or a biosimilar thereof. In some embodiments, the cancer is a HER2 low breast cancer, and the additional therapy is an anti-HER2 antibody or anti-HER2 antibody-drug conjugate. In some embodiments, the cancer is a HER2 low breast cancer, and the additional therapy is trastuzumab deruxtecan (e.g., fam-trastuzumab deruxtecan-nxki, or a biosimilar thereof).

In some embodiments, the JAK inhibitor is adelatinib, baricitinib, brepocitinib, deuruxolitinib, fedratinib (e.g., fedratinib dihydrochloride monohydrate), filgotinib (e.g., filgotinib maleate), gandotinib, gusacitinib, ilginatinib, izencitinib, jaktinib, momelotinib (e.g., momelotinib dihydrochloride), nezulcitinib, pacritinib (e.g., pacritinib citrate), peficitinib (e.g., peficitinib hydrobromide), ropsacitinib, ruxolitinib (e.g., ruxolitinib phosphate), tasocitinib (e.g., tofacitinib citrate), AT-9283, TQ-05105, or a combination thereof. In some embodiments, the JAK inhibitor is fedratinib (e.g., fedratinib dihydrochloride monohydrate), momelotinib (e.g., momelotinib dihydrochloride), pacritinib (e.g., pacritinib citrate), ruxolitinib (e.g., ruxolitinib phosphate), or a combination thereof. In some embodiments, the JAK inhibitor is fedratinib (e.g., fedratinib dihydrochloride monohydrate). In some embodiments, the JAK inhibitor is momelotinib (e.g., momelotinib dihydrochloride). In some embodiments, the JAK inhibitor is pacritinib (e.g., pacritinib citrate). In some embodiments, the JAK inhibitor is ruxolitinib (e.g., ruxolitinib phosphate).

In some embodiments, a JAK V617F mutation can be detected in a sample from the subject (e.g., detecting a JAK2 gene having a mutation corresponding to a V617F mutation in JAK2 protein and/or detecting a JAK2 protein having a V617F mutation). In some embodiments, the subject was determined (e.g., prior to administration of a compound of Formula (I) or (II)) to have a cancer having a JAK2 V617F mutation. In some embodiments, the subject was determined (e.g., prior to administration of a compound of Formula (I) or (II)) to have a cancer lacking a JAK2 V617F mutation.

In some embodiments, the cancer is post-MPN AML, M6-AML, or M7-AML, and the additional therapy or therapeutic agent is a JAK inhibitor (e.g., ruxolitinib (e.g., ruxolitinib phosphate), fedratinib (e.g., fedratinib dihydrochloride monohydrate), momelotinib (e.g., momelotinib dihydrochloride), or pacritinib (e.g., pacritinib citrate)). In some embodiments, the patient has received a previous line of therapy including a JAK inhibitor. In some embodiments, the patient has not received a previous line of therapy including a JAK inhibitor.

In some embodiments, the cancer is intermediate (e.g., intermediate-1 and/or intermediate-2) or high-risk myelofibrosis (e.g., primary myelofibrosis, post-essential thrombocythemia myelofibrosis, or post-polycythemia vera myelofibrosis), and the additional therapy or therapeutic agent is a JAK inhibitor (e.g., ruxolitinib (e.g., ruxolitinib phosphate), fedratinib (e.g., fedratinib dihydrochloride monohydrate), momelotinib (e.g., momelotinib dihydrochloride), or pacritinib (e.g., pacritinib citrate)). In some embodiments, the cancer is intermediate (e.g., intermediate-1 and/or intermediate-2) or high-risk myelofibrosis (e.g., primary myelofibrosis, post-essential thrombocythemia myelofibrosis, or post-polycythemia vera myelofibrosis), and the additional therapy or therapeutic agent is a JAK inhibitor (e.g., ruxolitinib (e.g., ruxolitinib phosphate), fedratinib (e.g., fedratinib dihydrochloride monohydrate), momelotinib (e.g., momelotinib dihydrochloride), or pacritinib (e.g., pacritinib citrate)) and a BET inhibitor (e.g., alobresib, apabetalone, mivebresib, pelabresib, trotabresib, ABBV-744, BI-2536, BMS-986158, INCB-057643, JAB-8263, ODM-207, PLX-2853, or ZEN-003694). In some embodiments, the cancer is intermediate (e.g., intermediate-1 and/or intermediate-2) or high-risk myelofibrosis (e.g., primary myelofibrosis, post-essential thrombocythemia myelofibrosis, or post-polycythemia vera myelofibrosis) with a JAK2 mutation (e.g., a JAK2 V617F mutation), and the additional therapy or therapeutic agent is a JAK inhibitor (e.g., ruxolitinib (e.g., ruxolitinib phosphate), fedratinib (e.g., fedratinib dihydrochloride monohydrate), momelotinib (e.g., momelotinib dihydrochloride), or pacritinib (e.g., pacritinib citrate)). In some embodiments, the cancer is intermediate (e.g., intermediate-1 and/or intermediate-2) or high-risk myelofibrosis (e.g., primary myelofibrosis, post-essential thrombocythemia myelofibrosis, or post-polycythemia vera myelofibrosis) with a JAK2 mutation (e.g., a JAK2 V617F mutation), and the additional therapy or therapeutic agent is a JAK inhibitor (e.g., ruxolitinib (e.g., ruxolitinib phosphate), fedratinib (e.g., fedratinib dihydrochloride monohydrate), momelotinib (e.g., momelotinib dihydrochloride), or pacritinib (e.g., pacritinib citrate)) and a BET inhibitor (e.g., alobresib, apabetalone, mivebresib, pelabresib, trotabresib, ABBV-744, BI-2536, BMS-986158, INCB-057643, JAB-8263, ODM-207, PLX-2853, or ZEN-003694). In some embodiments, the JAK inhibitor is fedratinib (e.g., fedratinib dihydrochloride monohydrate). In some embodiments, the JAK inhibitor is momelotinib (e.g., momelotinib dihydrochloride). In some embodiments, the JAK inhibitor is pacritinib (e.g., pacritinib citrate). In some embodiments, the JAK inhibitor is ruxolitinib (e.g., ruxolitinib phosphate). In some embodiments, the patient has received a previous line of therapy including a JAK inhibitor. In some embodiments, the patient has not received a previous line of therapy including a JAK inhibitor. In some embodiments, treatment effect can be measured by Spleen Volume Reduction (e.g., Spleen Volume Reduction of greater than or equal to 35% (SVR₃₅), for example, measured by MRI or CT), Total Symptom Score (e.g., Total Symptom Score reduction of greater than or equal to 50% (TSS₅₀), for example, measured by the Myelofibrosis Symptom Assessment Form (MFSAF) version 4.0), or both, such as at 24 weeks after beginning of treatment; see, e.g., Harrison, Claire, et al., New England Journal of Medicine 366.9 (2012): 787-798; and Verstovsek, Srdan, et al. New England Journal of Medicine 366.9 (2012): 799-807. In some embodiments, treatment effect can be measured (e.g., in addition to or instead of SVR₃₅ and/or TSS₅₀) by anemia response (e.g., measured by current International Working Group-Myeloproliferative Neoplasms Research and European LeukemiaNet (IWG-MRT/ELN) criteria), bone marrow fibrosis (e.g., according to the European Consensus Grading System through bone marrow biopsy, such as at 24 or 96 weeks after beginning of treatment), variant allele fraction (e.g., JAK2 V617F variant allele fraction), transfusion independence, overall survival, leukemia-free survival, change in physical functioning (e.g., measured by the physical functioning domain of the European Organization for Research and Treatment of Cancer (EORTC) Quality of Life Questionnaire (QLQ)-C₃₀ or death), change in fatigue (e.g., assessed using the Patient-Reported Outcomes Measurement Information System (PROMIS) Fatigue SF 7a), or a combination thereof.

In some embodiments, the KRas inhibitor is adagrasib, divarasib (GDC-6036), sotorasib, ARS-1620, ARS-3248, ARS-853, ASP-3082, ATG-012, BI-1701963, BI-1823911, BPI-421286, D-1553, ERAS-3490, GFH-925, JAB-21822, JDQ-443, LY-3537982, MRTX-1133, MRTX-1257, RMC-6236, RMC-6291, RSC-1255, or a combination thereof.

In some embodiments, a KRas mutation (e.g., a KRas G12C mutation or a KRas G12D mutation) can be detected in a sample from the subject (e.g., detecting a KRAS gene having a mutation corresponding to a G12C mutation or a G12D mutation in KRas protein and/or detecting a KRas protein having a G12C mutation or a G12D mutation). In some embodiments, the subject was determined (e.g., prior to administration of a compound of Formula (I) or (II)) to have a cancer having a KRas G12C mutation. In some embodiments, the subject was determined (e.g., prior to administration of a compound of Formula (I) or (II)) to have a cancer having a KRas G12D mutation.

In some embodiments, the cancer is a KRas mutant lung cancer (e.g., a KRas mutant NSCLC), a KRas mutant CRC, or a KRas mutant pancreatic cancer, and the additional therapy or therapeutic agent is a KRas inhibitor (e.g., adagrasib, divarasib (GDC-6036), sotorasib, ARS-1620, ARS-3248, ARS-853, ASP-3082, ATG-012, BI-1701963, BI-1823911, BPI-421286, D-1553, ERAS-3490, GFH-925, JAB-21822, JDQ-443, LY-3537982, MRTX-1133, MRTX-1257, RMC-6236, RMC-6291, or RSC-1255). In some embodiments, the cancer is a KRas mutant lung cancer (e.g., a KRas mutant NSCLC (e.g., a KRas G12C mutant NSCLC)), a KRas mutant CRC (e.g., a KRas G12C mutant CRC), or a KRas mutant pancreatic cancer (e.g., a KRas G12C mutant pancreatic cancer), and the additional therapy or therapeutic agent is adagrasib. In some embodiments, the cancer is a KRas mutant lung cancer (e.g., a KRas mutant NSCLC (e.g., a KRas G12C mutant NSCLC)), a KRas mutant CRC (e.g., a KRas G12C mutant CRC), or a KRas mutant pancreatic cancer (e.g., a KRas G12C mutant pancreatic cancer), and the additional therapy or therapeutic agent is divarasib. In some embodiments, the cancer is a KRas mutant lung cancer (e.g., a KRas mutant NSCLC (e.g., a KRas G12C mutant NSCLC)), a KRas mutant CRC (e.g., a KRas G12C mutant CRC), or a KRas mutant pancreatic cancer (e.g., a KRas G12C mutant pancreatic cancer), and the additional therapy or therapeutic agent is sotorasib. In some embodiments, the cancer is a KRas mutant lung cancer (e.g., a KRas mutant NSCLC (e.g., a KRas G12D mutant NSCLC)), a KRas mutant CRC (e.g., a KRas G12D mutant CRC), or a KRas mutant pancreatic cancer (e.g., a KRas G12D mutant pancreatic cancer), and the additional therapy or therapeutic agent is MRTX1133.

In some embodiments, the MEK inhibitor is avutometinib, binimetinib, cobimetinib (e.g., cobimetinib fumarate), mirdametinib, pimasertib, refametinib, selumetinib (e.g., selumetinib sulfate), trametinib (e.g., trametinib dimethyl sulfoxide), zapnometinib, FCN-159, GSK-1120212, NFX-179, TAK-733, or a combination thereof.

In some embodiments, a BRCA1 mutation can be detected in a sample from the subject (e.g., detecting a BRCA1 gene having a mutation and/or detecting a BRCA1 protein having a mutation). In some embodiments, the subject was determined (e.g., prior to administration of a compound of Formula (I) or (II)) to have a cancer having a BRCA1 mutation.

In some embodiments, a BRCA2 mutation can be detected in a sample from the subject (e.g., detecting a BRCA2 gene having a mutation and/or detecting a BRCA2 protein having a mutation). In some embodiments, the subject was determined (e.g., prior to administration of a compound of Formula (I) or (II)) to have a cancer having a BRCA2 mutation.

In some embodiments, the cancer is ovarian cancer (e.g., BRCA1 mutant ovarian cancer or BRCA2 mutant ovarian cancer, HGSOC (e.g., BRCA1 mutant HGSOC or BRCA2 mutant HGSOC), or LGSOC), and the additional therapy or therapeutic agent is a MEK inhibitor (e.g., binimetinib, cobimetinib, selumetinib, or trametinib). In some embodiments, the cancer is BRCA1 mutant ovarian cancer, and the additional therapy or therapeutic agent is a MEK inhibitor (e.g., binimetinib, cobimetinib, selumetinib, or trametinib). In some embodiments, the cancer is BRCA2 mutant ovarian cancer, and the additional therapy or therapeutic agent is a MEK inhibitor (e.g., binimetinib, cobimetinib, selumetinib, or trametinib). In some embodiments, the cancer is HGSOC (e.g., BRCA1 mutant HGSOC or BRCA2 mutant HGSOC), and the additional therapy or therapeutic agent is a MEK inhibitor (e.g., binimetinib, cobimetinib, selumetinib, or trametinib). In some embodiments, the cancer is LGSOC (e.g., BRCA1 mutant HGSOC or BRCA2 mutant HGSOC), and the additional therapy or therapeutic agent is a MEK inhibitor (e.g., binimetinib, cobimetinib, selumetinib, or trametinib).

In some embodiments, the cancer is a KRas mutant CRC (e.g., a KRas G12C mutant NSCLC), and the additional therapy or therapeutic agent is a MEK inhibitor (e.g., binimetinib, cobimetinib, selumetinib, or trametinib).

In some embodiments, the MET inhibitor is cabozantinib (e.g., cabozantinib S-malate), capmatinib (e.g., capmatinib hydrochloride), crizotinib, foritinib, glesatinib, gumarontinib, merestinib, pamufetinib, savolitinib, sitravatinib, tepotinib (e.g., tepotinib hydrochloride hydrate), vebreltinib, zanzalintinib (XL-092), amivantamab (e.g., amivantamab-vmjw, or a biosimilar thereof), emibetuzumab (or biosimilars thereof), RC-108, telisotuzumab vedotin (or biosimilars thereof), ABBV-400, ABN-401, AL-2846, AMG-337, SAR-125844, TQ-B-3139, or a combination thereof.

In some embodiments, a MET alteration can be detected in a sample from the subject (e.g., detecting a MET gene having an alteration (e.g., gene amplification or exon14 skipping) and/or detecting a MET protein having a mutation (e.g., exon14 skipping)). In some embodiments, the subject was determined (e.g., prior to administration of a compound of Formula (I) or (II)) to have a cancer having a MET alteration.

In some embodiments, the cancer is a MET-altered NCLC (e.g., MET amplified NSCLC or MET exon14 skipping NSCLC), and the additional therapy or therapeutic agent is a MET inhibitor (e.g., cabozantinib (e.g., cabozantinib S-malate), capmatinib (e.g., capmatinib hydrochloride), crizotinib, foritinib, glesatinib, gumarontinib, merestinib, pamufetinib, savolitinib, sitravatinib, tepotinib (e.g., tepotinib hydrochloride hydrate), vebreltinib, zanzalintinib (XL-092), amivantamab (e.g., amivantamab-vmjw, or a biosimilar thereof), emibetuzumab (or biosimilars thereof), RC-108, telisotuzumab vedotin (or biosimilars thereof), ABBV-400, ABN-401, AL-2846, AMG-337, SAR-125844, or TQ-B-3139). In some embodiments, the cancer is a MET-altered NCLC (e.g., MET amplified NSCLC or MET exon14 skipping NSCLC), and the additional therapy or therapeutic agent is capmatinib (e.g., capmatinib hydrochloride) or tepotinib (e.g., tepotinib hydrochloride hydrate). In some embodiments, the cancer is a MET-altered NCLC (e.g., MET amplified NSCLC or MET exon14 skipping NSCLC), and the additional therapy or therapeutic agent is telisotuzumab vedotin (or biosimilars thereof).

In some embodiments, the Hif2a inhibitor is belzutifan, AB-521, DFF-332, NKT-2152, PT-2399, or a combination thereof.

In some embodiments, the PARP inhibitor is fuzuloparib (fluzoparib), niraparib (e.g., niraparib tosylate monohydrate), olaparib, pamiparib, rucaparib (e.g., rucaparib camsylate), saruparib (AZD5305), senaparib, stenoparib, talazoparib (e.g., talazoparib tosylate), veliparib, CEP-9722, JPI-289, NMS-03305293, or a combination thereof. In some embodiments, the PARP inhibitor is a PARP1 inhibitor. In some embodiments, the PARP1 inhibitor is saruparib (AZD5305), NMS-03305293, or a combination thereof.

In some embodiments, the cancer is BRCA1 mutant breast cancer or BRCA2 mutant breast cancer, and the additional therapy or therapeutic agent is a PARP inhibitor (e.g., fuzuloparib (fluzoparib), niraparib (e.g., niraparib tosylate monohydrate), olaparib, pamiparib, rucaparib (e.g., rucaparib camsylate), saruparib (AZD5305), senaparib, stenoparib, talazoparib (e.g., talazoparib tosylate), veliparib, CEP-9722, JPI-289, or NMS-03305293). In some embodiments, the cancer is BRCA1 mutant breast cancer or BRCA2 mutant breast cancer, and the additional therapy or therapeutic agent is saruparib.

In some embodiments, the cancer is triple negative breast cancer, and the additional therapy or therapeutic agent is a PARP inhibitor (e.g., fuzuloparib (fluzoparib), niraparib (e.g., niraparib tosylate monohydrate), olaparib, pamiparib, rucaparib (e.g., rucaparib camsylate), saruparib (AZD5305), senaparib, stenoparib, talazoparib (e.g., talazoparib tosylate), veliparib, CEP-9722, JPI-289, or NMS-03305293). In some embodiments, the cancer is triple negative breast cancer, and the additional therapy or therapeutic agent is saruparib.

In some embodiments, the cancer is HGSOC (e.g., BRCA1 mutant HGSOC or BRCA2 mutant HGSOC), and the additional therapy or therapeutic agent is a PARP inhibitor (e.g., fuzuloparib (fluzoparib), niraparib (e.g., niraparib tosylate monohydrate), olaparib, pamiparib, rucaparib (e.g., rucaparib camsylate), saruparib (AZD5305), senaparib, stenoparib, talazoparib (e.g., talazoparib tosylate), veliparib, CEP-9722, JPI-289, or NMS-03305293). In some embodiments, the cancer is HGSOC (e.g., BRCA1 mutant HGSOC or BRCA2 mutant HGSOC), and the additional therapy or therapeutic agent is saruparib.

In some embodiments, the LSD1 inhibitor is bomedemstat, iadademstat, pulrodemstat, seclidemstat (HCI-2577), vafidemstat, GSK-2879552, INCB-059872, JBI-802, or a combination thereof.

In some embodiments, the BET inhibitor is alobresib, apabetalone, mivebresib, pelabresib, trotabresib, ABBV-744, BI-2536, BMS-986158, INCB-057643, JAB-8263, ODM-207, PLX-2853, ZEN-003694, or a combination thereof.

In some embodiments, the cancer is myelofibrosis (e.g., primary myelofibrosis, post-essential thrombocythemia myelofibrosis, or post-polycythemia vera myelofibrosis), and the additional therapy or therapeutic agent is a BET inhibitor (e.g., alobresib, apabetalone, mivebresib, pelabresib, trotabresib, ABBV-744, BI-2536, BMS-986158, INCB-057643, JAB-8263, ODM-207, PLX-2853, or ZEN-003694).

In some embodiments, the STING agonist is ulevostinag, ADU-S100, or a combination thereof.

In some embodiments, the telomerase inhibitor is imetelstat (e.g., imetelstat sodium).

In some embodiments, the TORC1/2 inhibitor is apitolisib, bimiralisib, dactolisib, deforolimus, everolimus, fosciclopirox (e.g., fosciclopirox sodium), gedatolisib, onatasertib, paxalisib, sapanisertib, sirolimus, sodium 2-hydroxylinoleate, temsirolimus, umirolimus, vistusertib, zandelisib, zotarolimus, BI-860585, CC-115, CLL-442, PF-04691502, or a combination thereof.

In some embodiments, the VEGFR inhibitor is apatinib, axitinib, cabozantinib (e.g., cabozantinib S-malate), catequentinib (alontinib), cediranib, dovitinib, famitinib, fruquintinib, glesatinib, ibcasertib, ilorasertib, lenvatinib (e.g., lenvatinib mesylate), lucitanib, nintedanib (e.g., nintedanib esylate), pamufetinib, pazopanib (e.g., pazopanib hydrochloride), regorafenib (e.g., regorafenib monohydrate), sitravatinib, sorafenib (e.g., sorafenib tosylate), sunitinib (e.g., sunitinib malate), surufatinib (sulfatinib), telatinib, tinengotinib, tivozanib (e.g., tivozanib hydrochloride monohydrate), vandetanib, vorolanib, zanzalintinib, olinvacimab (or biosimilars thereof), ramucirumab (or biosimilars thereof), CEP-11981, ENMD-2076, ODM-203, or a combination thereof.

In some embodiments, the chemotherapy is a platinum complex, a microtubule inhibitor (e.g., a microtubule destabilizer or a microtubule stabilizer), a topoisomerase inhibitor, a hypomethylating agent, or an antibody-drug conjugate including any thereof. In some embodiments, the platinum complex is carboplatin, cisplatin, lobaplatin, miriplatin, oxaliplatin, or a combination thereof. In some embodiments, the microtubule inhibitor is cabazitaxel, colchicine, desoxyepothilone B, docetaxel, eribulin, ixabepilone, nab-paclitaxel, paclitaxel, plinabulin, sabizabulin, tirbanibulin, vinblastine, vinflunine, vinorelbine, or a combination thereof. In some embodiments, the microtubule inhibitor is cabazitaxel, docetaxel, nab-paclitaxel, paclitaxel, or a combination thereof. In some embodiments, the topoisomerase inhibitor is aclarubicin, amsacrine, belotecan, camptothecin, daunorubicin, dexrazoxane, elliptinium, epirubicin, etoposide, gepotidacin, idarubicin, mitoxantrone, nemonoxacin, pirarubicin, pixantrone, razoxane, rubitecan, sobuzoxane, temozolomide, teniposide, topotecan, SN-38, or a combination thereof. In some embodiments, the hypomethylating agent is azacitidine, decitabine, or a combination thereof. In some embodiments, the chemotherapy is a platinum complex and a topoisomerase inhibitor (e.g., cisplatin and etoposide). In some embodiments, the antibody-drug conjugate including the microtubule inhibitor is belantamab mafodotin, brentuximab vedotin, cofetuzumab pelidotin, disitamab vedotin, enfortumab vedotin (e.g., enfortumab vedotin-ejfv, or a biosimilar thereof), mirvetuximab soravtansine (e.g., mirvetuximab soravtansine-gynx, or a biosimilar thereof), polatuzumab vedotin, telisotuzumab vedotin, tisotumab vedotin, trastuzumab emtansine (e.g., ado-trastuzumab emtansine, or a biosimilar thereof), tusamitamab ravtansine, upifitamab rilsodotin, zilovertamab vedotin, Alpha-Her2-pAF1-AS-269, BAT-8001, TAA-013, biosimilars thereof, or a combination thereof. In some embodiments, the antibody-drug conjugate including the microtubule inhibitor is enfortumab vedotin (e.g., enfortumab vedotin-ejfv, or a biosimilar thereof). In some embodiments, the antibody-drug conjugate including the microtubule inhibitor is mirvetuximab soravtansine (e.g., mirvetuximab soravtansine-gynx, or a biosimilar thereof). In some embodiments, the antibody-drug conjugate including the microtubule inhibitor is trastuzumab emtansine (e.g., ado-trastuzumab emtansine, or a biosimilar thereof). In some embodiments, the antibody-drug conjugate including the topoisomerase inhibitor is datopotamab deruxtecan, patritumab deruxtecan, sacituzumab govitecan (e.g., sacituzumab govitecan-hziy, or a biosimilar thereof), trastuzumab deruxtecan (fam-trastuzumab deruxtecan-nxki, or a biosimilar thereof), or a combination thereof. In some embodiments, the antibody-drug conjugate including the topoisomerase inhibitor is sacituzumab govitecan (e.g., sacituzumab govitecan-hziy, or a biosimilar thereof). In some embodiments, the antibody-drug conjugate including the topoisomerase inhibitor is trastuzumab deruxtecan (e.g., fam-trastuzumab deruxtecan-nxki, or a biosimilar thereof).

In some embodiments, the cancer is a lung cancer (e.g., NSCLC (e.g., squamous cell carcinoma)), and the additional therapy or therapeutic agent is a microtubule inhibitor (e.g., cabazitaxel, docetaxel, nab-paclitaxel, or paclitaxel). In some embodiments, the cancer is a lung cancer (e.g., NSCLC (e.g., squamous cell carcinoma)), and the additional therapy or therapeutic agent is docetaxel.

In some embodiments, the cancer is NSCLC (e.g., NSCLC with a MET amplification), and the additional therapy or therapeutic agent is telisotuzumab vedotin.

In some embodiments, the cancer is a lung cancer (e.g., NSCLC), and the additional therapy or therapeutic agent is a platinum complex (e.g., carboplatin, cisplatin, lobaplatin, miriplatin, or oxaliplatin) and anti-PD1 therapy.

In some embodiments, the cancer is a lung cancer (e.g., NSCLC), and the additional therapy or therapeutic agent is a platinum complex (e.g., carboplatin, cisplatin, lobaplatin, miriplatin, or oxaliplatin) and anti-PD-L1 therapy.

In some embodiments, the cancer is a lung cancer (e.g., NSCLC), and the additional therapy or therapeutic agent is a pemetrexed and anti-PD1 therapy.

In some embodiments, the cancer is a lung cancer (e.g., NSCLC), and the additional therapy or therapeutic agent is a pemetrexed and anti-PD-L1 therapy.

In some embodiments, the cancer is a lung cancer (e.g., SCLC), and the additional therapy or therapeutic agent is a platinum complex (e.g., carboplatin, cisplatin, lobaplatin, miriplatin, or oxaliplatin) and a topoisomerase inhibitor (e.g., aclarubicin, amsacrine, belotecan, camptothecin, daunorubicin, dexrazoxane, elliptinium, epirubicin, etoposide, gepotidacin, idarubicin, mitoxantrone, nemonoxacin, pirarubicin, pixantrone, razoxane, rubitecan, sobuzoxane, temozolomide, teniposide, topotecan, or SN-38). In some embodiments, the cancer is a lung cancer (e.g., SCLC), and the additional therapy or therapeutic agent is carboplatin and etoposide. In some embodiments, the cancer is a lung cancer (e.g., SCLC), and the additional therapy or therapeutic agent is cisplatin and etoposide.

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is a microtubule inhibitor (e.g., cabazitaxel, docetaxel, nab-paclitaxel, or paclitaxel). In some embodiments, the cancer is triple negative breast cancer, and the additional therapy or therapeutic agent is a microtubule inhibitor (e.g., cabazitaxel, docetaxel, nab-paclitaxel, or paclitaxel). In some embodiments, the cancer is triple negative breast cancer, and the additional therapy or therapeutic agent is nab-paclitaxel or paclitaxel.

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is a microtubule inhibitor (e.g., cabazitaxel, docetaxel, nab-paclitaxel, or paclitaxel) and anti-PD1 therapy.

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is a microtubule inhibitor (e.g., cabazitaxel, docetaxel, nab-paclitaxel, or paclitaxel) and anti-PD-L1 therapy.

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is capecitabine.

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is sacituzumab govitecan (e.g., sacituzumab govitecan-hziy, or a biosimilar thereof).

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is trastuzumab deruxtecan (e.g., fam-trastuzumab deruxtecan-nxki, or a biosimilar thereof).

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is trastuzumab (e.g., HERCEPTIN® (trastuzumab), or a biosimilar thereof (e.g., FACEPTOR® (trastuzumab), HERTICAD® (trastuzumab), TUZNUE® (trastuzumab), ZERCEPAC® (trastuzumab), trastuzumab-anns, trastuzumab-dkst, trastuzumab-dttb, trastuzumab-pkrb, trastuzumab-qyyp, EG-12014, or TX-05)).

In some embodiments, the cancer is hormone receptor positive breast cancer, and the additional therapy or therapeutic agent is hormone therapy (e.g., tamoxifen, toremifene, or a combination thereof).

In some embodiments, the cancer is hormone receptor positive breast cancer, and the additional therapy or therapeutic agent is a selective estrogen receptor degrader (SERD) (e.g., fulvestrant, elacestrant, or a combination thereof).

In some embodiments, the cancer is a HER2+ breast cancer (e.g., HER2+ breast cancer with ER expression, HER2+ breast cancer without ER expression), and the additional therapy or therapeutic agent is trastuzumab deruxtecan (e.g., fam-trastuzumab deruxtecan-nxki, or a biosimilar thereof) or trastuzumab emtansine (e.g., KADCYLA® (trastuzumab emtansine), or a biosimilar thereof (e.g., UJVTRA® (trastuzumab emtansine))).

In some embodiments, the cancer is a HER2 low breast cancer, and the additional therapy or therapeutic agent is trastuzumab deruxtecan (e.g., fam-trastuzumab deruxtecan-nxki, or a biosimilar thereof).

In some embodiments, the cancer is a head and neck cancer, and the additional therapy or therapeutic agent is a microtubule inhibitor (e.g., cabazitaxel, docetaxel, nab-paclitaxel, or paclitaxel). In some embodiments, the cancer is a head and neck cancer, and the additional therapy or therapeutic agent is docetaxel.

In some embodiments, the cancer is a cervical cancer, and the additional therapy or therapeutic agent is a microtubule inhibitor (e.g., cabazitaxel, docetaxel, nab-paclitaxel, or paclitaxel).

In some embodiments, the cancer is an endometrial cancer, and the additional therapy or therapeutic agent is a microtubule inhibitor (e.g., cabazitaxel, docetaxel, nab-paclitaxel, or paclitaxel).

In some embodiments, the cancer is a prostate cancer, and the additional therapy or therapeutic agent is a microtubule inhibitor (e.g., cabazitaxel, docetaxel, nab-paclitaxel, or paclitaxel).

In some embodiments, the cancer is an ovarian cancer (e.g., HGSOC) and the additional therapy or therapeutic agent is a microtubule inhibitor (e.g., cabazitaxel, docetaxel, nab-paclitaxel, or paclitaxel). In some embodiments, the cancer is an ovarian cancer (e.g., HGSOC) and the additional therapy or therapeutic agent is a microtubule inhibitor (e.g., cabazitaxel, docetaxel, nab-paclitaxel, or paclitaxel) and a platinum complex (e.g., carboplatin, cisplatin, lobaplatin, miriplatin, or oxaliplatin). In some embodiments, the cancer is an ovarian cancer (e.g., HGSOC) and the additional therapy or therapeutic agent is nab-paclitaxel or paclitaxel and carboplatin.

In some embodiments, the cancer is a pancreatic cancer, and the additional therapy or therapeutic agent is a microtubule inhibitor (e.g., cabazitaxel, docetaxel, nab-paclitaxel, or paclitaxel). In some embodiments, the cancer is a pancreatic cancer, and the additional therapy or therapeutic agent is a microtubule inhibitor (e.g., cabazitaxel, docetaxel, nab-paclitaxel, or paclitaxel) and gemcitabine.

In some embodiments, the cancer is a bladder cancer (e.g., bladder urothelial carcinoma), and the additional therapy or therapeutic agent is enfortumab vedotin (e.g., enfortumab vedotin-ejfv, or a biosimilar thereof).

In some embodiments, the cancer is a bladder cancer (e.g., bladder urothelial carcinoma), and the additional therapy or therapeutic agent is sacituzumab govitecan (e.g., sacituzumab govitecan-hziy, or a biosimilar thereof).

In some embodiments, the cancer is triple negative breast cancer, and the additional therapy or therapeutic agent is sacituzumab govitecan (e.g., sacituzumab govitecan-hziy, or a biosimilar thereof).

In some embodiments, folate receptor positivity status can be detected in a sample from the subject (e.g., via immunohistochemistry (IHC) and/or fluorescent in situ hybridization (FISH)). In some embodiments, the subject was determined (e.g., prior to administration of a compound of Formula (I) or (II)) to have a cancer that is folate receptor positive.

In some embodiments, the cancer is folate receptor positive ovarian cancer (e.g., folate receptor positive HGSOC), and the additional therapy or therapeutic agent is mirvetuximab soravtansine (e.g., mirvetuximab soravtansine-gynx, or a biosimilar thereof).

In some embodiments the immunomodulatory imide drug is thalidomide, lenalidomide, pomalidomide, iberdomide, avadomide, CC-99282, or a combination thereof.

In some embodiments, the anti-PD1 therapy is balstilimab, budigalimab, cadonilimab, camrelizumab, cemiplimab (e.g., cemiplimab-rwlc, or a biosimilar thereof), cetrelimab, dostarlimab (e.g., dostarlimab-gxly, or a biosimilar thereof), ezabenlimab, geptanolimab, ivonescimab, nivolumab (e.g., OPDIVO® (nivolumab), or a biosimilar thereof), nofazinlimab, pembrolizumab (e.g., KEYTRUDA® (pembrolizumab), or a biosimilar thereof), penpulimab, pidilizumab, pimivalimab, prolgolimab, pucotenlimab, retifanlimab (e.g., retifanlimab-dlwr, or a biosimilar thereof), rilvegostomig, rosnilimab, rulonilimab, sasanlimab, serplulimab, sintilimab (e.g., TYVYT® (sintilimab), or a biosimilar thereof), spartalizumab, tebotelimab, tislelizumab, toripalimab, volrustomig, vudalimab, zimberelimab, QL-1604, HX-009, INCB-086550, RG-6139, BAT-1306, SG-001, biosimilars thereof, or a combination thereof.

In some embodiments, the PD-L1 inhibitor is INCB-086550.

In some embodiments, the anti-PD-L1 therapy is adebrelimab, atezolizumab (e.g., TECENTRIQ® (atezolizumab), or a biosimilar thereof), avelumab (e.g., BAVENCIO® (avelumab), or a biosimilar thereof), bintrafusp alfa, cosibelimab, danburstotug, durvalumab (e.g., TIFINZI® (durvalumab), or a biosimilar thereof), envafolimab (e.g., ENWEIDA® (envafolimab), or a biosimilar thereof), erfonrilimab, pacmilimab, socazolimab, sugemalimab (e.g., CEJEMLY® (sugemalimab), or a biosimilar thereof), A-167, APL-502, AUPM-170, BNT-311, SHR-1701, biosimilars thereof, or a combination thereof.

In some embodiments, the additional therapy is radiotherapy.

In some embodiments, the cancer is head and neck cancer (e.g., head and neck squamous cell carcinoma), and the additional therapy is radiotherapy.

In some embodiments, the additional therapy includes a BRaf inhibitor and a MEK inhibitor. For example, the additional therapy can include dabrafenib and trametinib, vemurafenib and cobimetinib, or encorafenib and binimetinib.

Exemplary descriptions of agents in combination with BCL-2 family inhibitors can be found in: Hikita, Hayato, et al. Hepatology 52.4 (2010): 1310-1321; Chen, Jun, et al. Molecular Cancer Therapeutics 10.12 (2011): 2340-2349; Inuzuka, Hiroyuki, et al. Nature 471.7336 (2011): 104-109; Wertz, Ingrid E., et al. Nature 471.7336 (2011): 110-114; Tan, Nguyen, et al. Clinical Cancer Research 17.6 (2011): 1394-1404; Wong, Maureen, et al. Molecular Cancer Therapeutics 11.4 (2012): 1026-1035; Corcoran, Ryan B., et al. Cancer Cell 23.1 (2013): 121-128; Waibel, Michaela, et al. Cell Reports 5.4 (2013): 1047-1059; Frederick, Dennie T., et al. PloS One 9.7 (2014): e101286; Vlahovic, Gordana, et al. Investigational New Drugs 32.5 (2014): 976-984; Faber, Anthony C., et al. Cancer Discovery 4.1 (2014): 42-52; Leverson, Joel D., et al. Science Translational Medicine 7.279 (2015): 279ra40-279ra40; Guo, Jun, et al. PLoS One 10.3 (2015): e0114363; Lheureux, Stephanie, et al. International Journal of Cancer 136.5 (2015): E340-E350; Zoeller, Jason J., et al. Cancer Research 76.14_Supplement (2016): 4358-4358; Weeden, Clare E., et al. Oncogene 37.32 (2018): 4475-4488; Lucantoni, Federico, et al. Cell Death & Disease 9.2 (2018): 1-13; Iavarone, Claudia, et al. Molecular Cancer Therapeutics 18.3 (2019): 642-655; Fleury, Hubert, et al. Nature Communications 10.1 (2019): 1-15; Lohard, Steven, et al. Nature Communications 11.1 (2020): 1-16; Bertino, Erin M., et al. Clinical Cancer Research 27.6 (2021): 1604-1611; Guo, Ting, et al. Aging 13.15 (2021): 19750; Puglisi, Martina, et al. Future Oncology 17.21 (2021): 2747-2758; Harrison, Claire N., et al. Journal of Clinical Oncology 40.15 (2022): 1671; Kohler, Jens, et al. Molecular Cancer Therapeutics 20.4 (2021): 641-654; Jaaks, Patricia, et al. Nature 603.7899 (2022): 166-173; Sobol, Benjamin, et al. International Journal of Molecular Sciences 23.14 (2022): 7850; Passamonti, J Clin Oncol 40, (2022) (suppl 16; abstr 7015); Qin, J Clin Oncol 40, (2022) (suppl 16; abstr e20612); Potter, Danielle S., et al. Cancer Research 82.12_Supplement (2022): 3691-3691; and Shebl, Bassem, et al. Cancer Research 82.12_Supplement (2022): 3888-3888.

Also provided herein is a method of treating cancer, comprising administering to a subject in need thereof (a) a compound of Formula (I) or (II), or a pharmaceutically acceptable salt thereof, and (b) an additional therapeutic agent, for simultaneous, separate or sequential use for the treatment of cancer, wherein the amounts of the compound of Formula (I) or (II), or a pharmaceutically acceptable salt thereof, and the additional therapeutic agent are together effective in treating the cancer. In some embodiments, the method comprises administering (c) at least one pharmaceutically acceptable carrier.

These additional therapeutic agents may be administered with one or more doses of the compound of Formula (I) or (II), or a pharmaceutically acceptable salt thereof, or pharmaceutical composition thereof, as part of the same or separate dosage forms, via the same or different routes of administration, and/or on the same or different administration schedules according to standard pharmaceutical practice known to one skilled in the art. In some embodiments, the compound of Formula (I) or (II), or a pharmaceutically acceptable salt thereof, and the additional therapeutic agent are administered simultaneously as separate dosages. In some embodiments, a compound of Formula (I) or (II), or a pharmaceutically acceptable salt thereof, and the additional therapeutic agent are administered as separate dosages sequentially in any order, in jointly therapeutically effective amounts, e.g., in daily or intermittently dosages. In some embodiments, the compound of Formula (I) or (II), or a pharmaceutically acceptable salt thereof, and the additional therapeutic agent are administered simultaneously as a combined dosage.

Also provided herein is (i) a pharmaceutical combination for treating a cancer in a subject in need thereof, which comprises (a) a compound of Formula (I) or (II), or a pharmaceutically acceptable salt thereof, and (b) at least one additional therapeutic agent (e.g., any of the exemplary additional therapeutic agents described herein or known in the art), for simultaneous, separate or sequential use for the treatment of cancer, wherein the amounts of the compound of Formula (I) or (II), or a pharmaceutically acceptable salt thereof, and of the additional therapeutic agent are together effective in treating the cancer; (ii) a pharmaceutical composition comprising such a combination; (iii) the use of such a combination for the preparation of a medicament for the treatment of cancer; and (iv) a commercial package or product comprising such a combination as a combined preparation for simultaneous, separate or sequential use; and to a method of treatment of cancer in a subject in need thereof. In some embodiments, the pharmaceutical combination comprises (c) at least one pharmaceutically acceptable carrier.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the terms “subject,” “individual,” or “patient,” are used interchangeably, refers to any animal, including mammals such as mice, rats, other rodents, rabbits, dogs, cats, swine, cattle, sheep, horses, primates, and humans. In some embodiments, the subject is a human. In some embodiments, the subject has experienced and/or exhibited at least one symptom of the disease, disorder, or condition to be treated and/or prevented.

In some embodiments, the subject is a pediatric subject.

The term “pediatric subject” as used herein refers to a subject under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric subject is from birth through the first 28 days of life, from 29 days of age to less than two years of age, from two years of age to less than 12 years of age, or 12 years of age through 21 years of age (up to, but not including, the twenty-second birthday). In some embodiments, a pediatric subject is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age.

The term “preventing” as used herein means to delay the onset, recurrence or spread, in whole or in part, of the disease or condition as described herein, or a symptom thereof.

The term “regulatory agency” refers to a country's agency for the approval of the medical use of pharmaceutical agents with the country. For example, a non-limiting example of a regulatory agency is the U.S. Food and Drug Administration (FDA).

The phrase “therapeutically effective amount” means an amount of compound that, when administered to a subject in need thereof, is sufficient to (i) treat a disease, disorder, or condition, (ii) attenuate, ameliorate, or eliminate one or more symptoms of the particular disease, disorder, or condition, or (iii) delay the onset of one or more symptoms of the particular disease, disorder, or condition described herein. The amount of a compound of Formula (I) or (II), or a pharmaceutically acceptable salt thereof, that will correspond to such an amount will vary depending upon factors such as the particular compound, disease condition and its severity, the identity (e.g., weight) of the subject in need of treatment, but can nevertheless be routinely determined by one skilled in the art.

The phrase “effective amount” of a compound of Formula (I) or (II), as used herein, means an amount of the compound that, when administered to a cell, in vitro or in vivo, is sufficient to reduce proliferation of the cell or to kill the cell. The amount of a compound of Formula (I) or (II), or a pharmaceutically acceptable salt thereof, that will correspond to such an amount will vary depending upon factors such as the particular compound and genetics of the cell to be treated, but can nevertheless be routinely determined by one skilled in the art.

### Pharmaceutical Compositions and Administration

### General

In some embodiments, a compound of Formula (I) or (II), or a pharmaceutically acceptable salt thereof, is administered as a pharmaceutical composition that includes the compound, or a pharmaceutically acceptable salt thereof, and one or more pharmaceutically acceptable excipients, and optionally one or more additional therapeutic agents as described herein.

In some embodiments, the compounds can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a compound as described herein in the range of 0.005% to 100% with the balance made up from non-toxic excipient may be prepared. The contemplated compositions may contain 0.001%-100% of a compound provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

### Routes of Administration and Composition Components

In some embodiments, a compound of Formula (I) or (II), or a pharmaceutically acceptable salt thereof, as described herein, or a pharmaceutical composition thereof, can be administered to a subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, intravitreal, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

In some embodiments, a compound of Formula (I) or (II), or a pharmaceutically acceptable salt thereof, as described herein, or a pharmaceutical composition thereof, can be administered orally to a subject in need thereof. Without being bound by any particular theory, it is believed that oral dosing (e.g., versus IV dosing) can be preferred by patients for convenience, perception of efficacy, and/or past experience.

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the compound described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein, or a pharmaceutical composition thereof, are suitable for local delivery to the digestive or GI tract by way of oral administration (e.g., solid or liquid dosage forms.).

Solid dosage forms for oral administration include capsules, tablets, pills, powders, and granules. In such solid dosage forms, the compound is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a compound, or pharmaceutically acceptable salt thereof, as provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives, or the like. In another solid dosage form, a powder, marume, solution or suspension (e.g., in propylene carbonate, vegetable oils, PEGs, poloxamer 124 or triglycerides) is encapsulated in a capsule (gelatin or cellulose base capsule). Unit dosage forms in which one or more compounds provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the compounds to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier, and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic, or amphoteric surfactant. As with other carriers or vehicles, an ointment base should be inert, stable, nonirritating, and non-sensitizing.

In any of the foregoing embodiments, pharmaceutical compositions described herein can include one or more one or more of the following: lipids, interbilayer crosslinked multilamellar vesicles, biodegradable poly(D,L-lactic-co-glycolic acid) [PLGA]-based or poly anhydride-based nanoparticles or microparticles, and nanoporous particle-supported lipid bilayers.

### Dosages

The dosages may be varied depending on the requirement of the patient, the severity of the condition being treated, and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 1 mg/kg; from about 0.1 mg/kg to about 0.5 mg/kg).

### Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

The term “acceptable” with respect to a formulation, composition, or ingredient, as used herein, means having no persistent detrimental effect on the general health of the subject being treated.

“API” refers to an active pharmaceutical ingredient.

The term “excipient” or “pharmaceutically acceptable excipient” means a pharmaceutically acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid, and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The term “pharmacologically acceptable salts” is not specifically limited as far as it can be used in medicaments. Examples of a salt that the compounds described herein form with a base include the following: salts thereof with inorganic bases such as sodium, potassium, magnesium, calcium, and aluminum; salts thereof with organic bases such as methylamine, ethylamine, and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to a subject. Multiple techniques of administering a compound exist in the art including, but not limited to: rectal, oral, intravenous, aerosol, parenteral, ophthalmic, pulmonary, and topical administration.

### Compound Preparation

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as R. Larock, Comprehensive Organic Transformations, VCH Publishers (1989); L. Fieser and M. Fieser, Fieser and Fieser's Reagents for Organic Synthesis, John Wiley and Sons (1994); Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

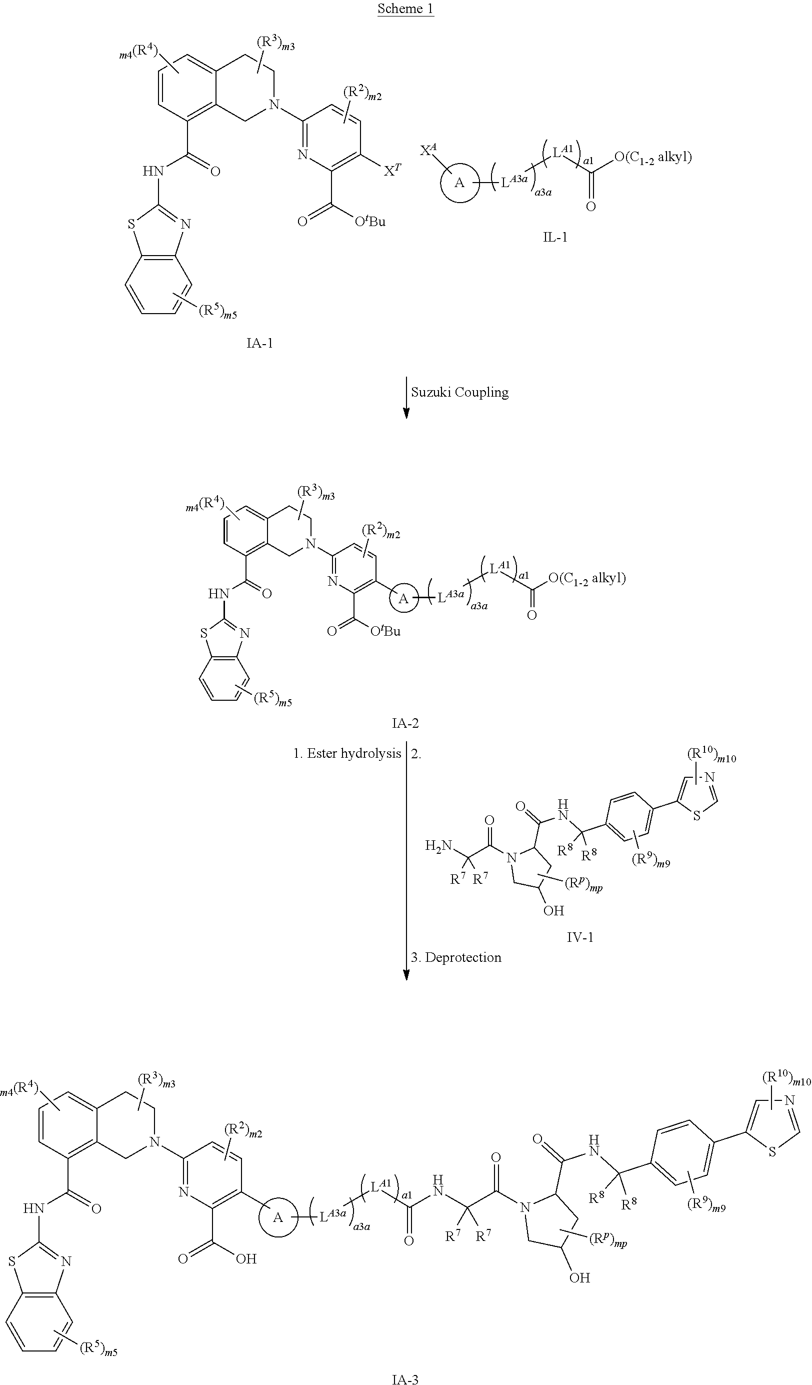

Scheme 1 depicts the synthesis of Compound IA-3, which is a compound of Formula (I), wherein R¹ is C(O)OH; L is (L-1) (wherein a3b is 1, and L^{A3b} is C(═O)); VBM is (VI) as defined in Formula (I); and each remaining variable is defined in accordance with Formula (I). Compound IA-1 is reacted with Compound I-L1 under standard conditions for Suzuki coupling to provide Compound IA-2, wherein one of X^{A} and X^{T} is B(OH)₂ or Bpin; the other of X^{A} and X^{T} is —Br; each remaining variable in IA-1 is defined in accordance with Formula (I); and each remaining variable in IL-1 is defined in accordance with Formula (I) and Formula (L-1). Hydrolysis of the C₁₂ alkyl ester in IA-2 provides the corresponding carboxylic acid which is then reacted with I-V1 under standard conditions for amide bond formation, wherein each variable in I-V1 is defined in accordance with Formula (I). Removal of the tert-butyl group under standard conditions for deprotection then provides Compound IA-3.

### Examples

In some of the examples disclosed herein, the final product of a described chemical reaction sequence is structurally depicted with an enhanced stereochemical or 1 notation at one stereogenic center. In some such examples, in the chemical name of the same compound, this stereogenic center is assigned a tentative configuration (e.g., (R)- or (S)-) based on the wedge/dash representation of the structural formula. However, this stereogenic center should be understood to have a configuration consistent with the or 1 notation. Specifically, this stereogenic center has been resolved, but its specific configuration has not been determined. Accordingly, unless otherwise specified, starting materials and intermediates leading to this compound incorporate the or 1 notation at this stereogenic center, notwithstanding the tentative assignments provided in their chemical names.

For example, Compound 134b in Example 38 is a single stereoisomer selected from:
6-[8-(1,3-benzothiazol-2-ylcarbamoyl)-3,4-dihydro-1H-isoquinolin-2-yl]-3-[3-[4-[(2R)-4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-methyl-4-oxo-butyl]phenoxy]-2-methyl-phenyl]pyridine-2-carboxylic acid; and
6-[8-(1,3-benzothiazol-2-ylcarbamoyl)-3,4-dihydro-1H-isoquinolin-2-yl]-3-[3-[4-[(2S)-4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-methyl-4-oxo-butyl]phenoxy]-2-methyl-phenyl]pyridine-2-carboxylic acid.

The intermediate product provided in Step A of the same example incorporates the or 1 notation. It is therefore a single stereoisomer selected from:
tert-butyl 6-[8-(1,3-benzothiazol-2-ylcarbamoyl)-3,4-dihydro-1H-isoquinolin-2-yl]-3-[3-[4-[(2R)-4-ethoxy-2-methyl-4-oxo-butyl]phenoxy]-2-methyl-phenyl]pyridine-2-carboxylate; and
tert-butyl 6-[8-(1,3-benzothiazol-2-ylcarbamoyl)-3,4-dihydro-1H-isoquinolin-2-yl]-3-[3-[4-[(2S)-4-ethoxy-2-methyl-4-oxo-butyl]phenoxy]-2-methyl-phenyl]pyridine-2-carboxylate.

For further clarification, a chemical name that takes into account the or 1 notation is provided for the final product having an enhanced stereochemical or 1 notation at one stereogenic center. This chemical name takes into account the or 1 notation, and it is enclosed in brackets (i.e., “[ ]”). In these chemical names, the prefix “rel” means that the stereochemical configuration shown in a chemical name is relative. To illustrate, when a compound contains one stereogenic center, and its chemical name starts with the prefix “rel,” then this stereogenic center is resolved, but its absolute configuration is either (R)- or (S)-. As such, it should be labelled with an or 1 enhanced stereochemical notation in its corresponding structure.

When a stereogenic center is labelled with an asterisk (“*”) in a chemical name of a compound having more than one stereogenic center, the stereogenic center labeled with the asterisk is resolved, but its absolute configuration is either (R)- or (S)-. As such, in a chemical name wherein one stereogenic center is labelled with the asterisk, that stereocenter should be labelled with an or 1 enhanced stereochemical notation in its corresponding structure.

For example, the chemical name containing an asterisk for Compound 134b is:

[6-(8-(benzo[d]thiazol-2-ylcarbamoyl)-3,4-dihydroisoquinolin-2(1H)-yl)-3-(3-(4-((R*)-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-methyl-4-oxobutyl)phenoxy)-2-methylphenyl)picolinic acid], as included in Example 38.

### Example 1. Preparation of Compound 105a

### 6-(8-(Benzo[d]thiazol-2-ylcarbamoyl)-3,4-dihydroisoquinolin-2(1H)-yl)-3-(3-((6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)oxy)-2-methylphenyl)picolinic acid

### Step A. Methyl 6-(3-bromo-2-methylphenoxy)hexanoate

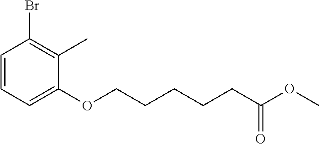 [SMILES: COC(=O)CCCCCOc1cccc(Br)c1C]

To a solution of methyl 6-bromohexanoate (5 g, 23.91 mmol, 1 eq) in ACN (50 mL) was added potassium carbonate (9.92 g, 71.74 mmol, 3 eq.) and 3-bromo-2-methyl-phenol (4.47 g, 23.91 mmol, 1 eq.). The mixture was stirred at 60° C. for 2 h and then the reaction mixture was concentrated under reduced pressure. The residue was diluted with water (50 mL) and extracted with ethyl acetate (20 mL×3). The combined organic layers were washed with brine (20 mL×3), dried over anhydrous sodium sulfate, filtered, and concentrated under reduced pressure to provide a residue that was purified by silica gel chromatography (heptane/ethyl acetate gradient) to afford the title compound (12 g, 19.04 mmol, 79.60% yield). ¹H NMR (400 MHz, CDCl₃) δ 7.86 (d, J=7.6 Hz, 1H), 7.74 (dd, J=7.6, 16.0 Hz, 2H), 7.51-7.45 (m, 1H), 7.42-7.33 (m, 4H), 7.09 (t, J=8.0 Hz, 1H), 6.88 (d, J=8.8 Hz, 1H), 6.80 (d, J=8.4 Hz, 1H), 6.70 (d, J=7.6 Hz, 1H), 5.06 (s, 2H), 4.11 (s, 2H), 4.00 (t, J=6.0 Hz, 2H), 3.08 (s, 2H), 2.40 (t, J=7.6 Hz, 2H), 2.11 (s, 3H), 1.83 (d, J=7.6 Hz, 2H), 1.77-1.71 (m, 2H), 1.58 (d, J=6.8 Hz, 2H), 1.16-1.13 (m, 9H).

### Step B. tert-Butyl 6-(8-(benzo[d]thiazol-2-ylcarbamoyl)-3,4-dihydroisoquinolin-2(1H)-yl)-3-(3-((6-methoxy-6-oxohexyl)oxy)-2-methylphenyl)picolinate

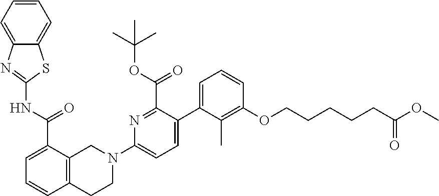 [SMILES: COC(=O)CCCCCOc1cccc(-c2ccc(N3CCc4cccc(C(=O)Nc5nc6ccccc6s5)c4C3)nc2C(=O)OC(C)(C)C)c1C]

To a solution of methyl 6-(3-bromo-2-methyl-phenoxy)hexanoate (500 mg, 793.14 μmol, 1 eq.) in 1,4-dioxane (5 mL) was added cataCXium-A-Pd-G3 (115.52 mg, 158.63 μmol, 0.2 eq.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (485.84 mg, 793.14 μmol, 1 eq.) and an aqueous solution of potassium carbonate (1.5 M, 1.59 mL, 3 eq.). The mixture was heated under microwave irradiation at 100° C. for 1 h and then concentrated under reduced pressure. The residue was diluted with water (10 mL) and extracted with ethyl acetate (10 mL×3). The combined organic layers were washed with brine (10 mL×3), dried over anhydrous sodium sulfate, filtered, and concentrated under reduced pressure to provide a residue that was purified by silica gel chromatography (heptane/ethyl acetate gradient) to afford the title compound (450 mg, 603.64 μmol, 76.11% yield). MS (ESI) m/z: 721.7 [M+H]⁺.

### Step C. 6-(3-(6-(8-(Benzo[d]thiazol-2-ylcarbamoyl)-3,4-dihydroisoquinolin-2(1H)-yl)-2-(tert-butoxycarbonyl)pyridin-3-yl)-2-methylphenoxy)hexanoic acid

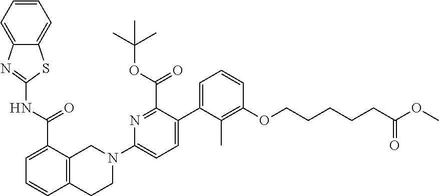 [SMILES: COC(=O)CCCCCOc1cccc(-c2ccc(N3CCc4cccc(C(=O)Nc5nc6ccccc6s5)c4C3)nc2C(=O)OC(C)(C)C)c1C]

To a solution of tert-butyl 6-[8-(1,3-benzothiazol-2-ylcarbamoyl)-3,4-dihydro-1H-isoquinolin-2-yl]-3-[3-(6-methoxy-6-oxo-hexoxy)-2-methyl-phenyl]pyridine-2-carboxylate (400 mg, 554.88 umol, 1 eq.) in THF (4 mL) was added lithium hydroxide monohydrate (69.85 mg, 1.66 mmol, 3 eq.) and water (1.00 g, 55.51 mmol, 1 mL, 100.04 eq.). The mixture was stirred at 25° C. for 12 h then diluted with water (10 mL) and extracted with ethyl acetate (10 mL×3). The combined organic layers were washed with water (10 mL×3), dried over anhydrous sodium sulfate, filtered, and concentrated under reduced pressure to afford the title compound (350 mg, crude) that was used without further purification. ¹H NMR (400 MHz, CDCl3) δ 7.86 (d, J=7.6 Hz, 1H), 7.74 (dd, J=7.6, 16.1 Hz, 2H), 7.51-7.45 (m, 1H), 7.42-7.33 (m, 4H), 7.09 (t, J=8.0 Hz, 1H), 6.88 (d, J=8.8 Hz, 1H), 6.80 (d, J=8.4 Hz, 1H), 6.70 (d, J=7.6 Hz, 1H), 5.06 (s, 2H), 4.11 (s, 2H), 4.00 (t, J=6.0 Hz, 2H), 3.08 (s, 2H), 2.40 (t, J=7.6 Hz, 2H), 2.11 (s, 3H), 1.83 (d, J=7.6 Hz, 2H), 1.77-1.71 (m, 2H), 1.58 (d, J=6.8 Hz, 2H), 1.16-1.13 (m, 9H).

### Step D. tert-Butyl 6-(8-(benzo[d]thiazol-2-ylcarbamoyl)-3,4-dihydroisoquinolin-2(1H)-yl)-3-(3-((6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)oxy)-2-methylphenyl)picolinate

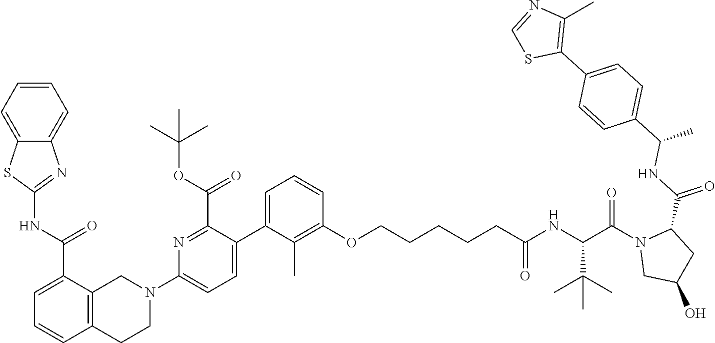 [SMILES: Cc1ncsc1-c1ccc([C@H](C)NC(=O)[C@@H]2C[C@@H](O)CN2C(=O)[C@@H](NC(=O)CCCCCOc2cccc(-c3ccc(N4CCc5cccc(C(=O)Nc6nc7ccccc7s6)c5C4)nc3C(=O)OC(C)(C)C)c2C)C(C)(C)C)cc1]

To a solution of 6-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]hexanoic acid (300 mg, 424.42 μmol, 1 eq) in DMF (5 mL) was added HATU (161.38 mg, 424.42 μmol, 1 eq.), DIEA (164.56 mg, 1.27 mmol, 221.78 μL, 3 eq.) and (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (377.38 mg, 848.84 μmol, 2 eq.). The mixture was stirred at 25° C. for 2 h. The mixture was diluted with water (10 mL) and extracted with ethyl acetate (10 mL×3). The combined organic layers were washed with water (10 mL×3), dried over anhydrous sodium sulfate, filtered, and concentrated under reduced pressure to afford the title compound (450 mg, crude) that was used without further purification. MS (ESI) m/z: 1133.4 [M+H]⁺.

### Step E. 6-(8-(benzo[d]thiazol-2-ylcarbamoyl)-3,4-dihydroisoquinolin-2(1H)-yl)-3-(3-((6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)oxy)-2-methylphenyl)picolinic acid

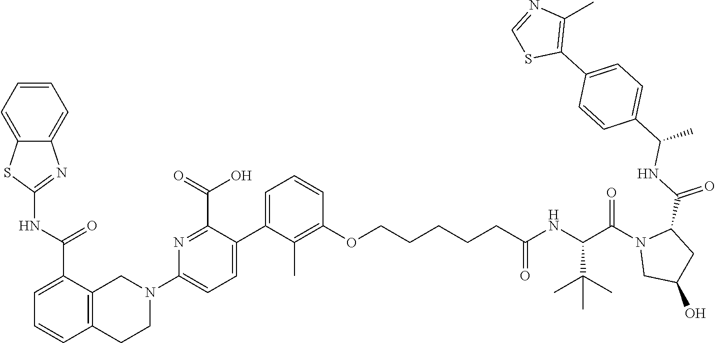 [SMILES: Cc1ncsc1-c1ccc([C@H](C)NC(=O)[C@@H]2C[C@@H](O)CN2C(=O)[C@@H](NC(=O)CCCCCOc2cccc(-c3ccc(N4CCc5cccc(C(=O)Nc6nc7ccccc7s6)c5C4)nc3C(=O)O)c2C)C(C)(C)C)cc1]

To a solution of tert-butyl 6-[8-(1,3-benzothiazol-2-ylcarbamoyl)-3,4-dihydro-1H-isoquinolin-2-yl]-3-[3-[6-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-6-oxo-hexoxy]-2-methyl-phenyl]pyridine-2-carboxylate (400 mg, 352.91 μmol, 1 eq.) in DCM (1 mL) was added TFA (402.4 mg, 3.53 mmol, 270.0 μL, 10 eq.). The mixture was stirred at 25° C. for 3 h. The reaction mixture was concentrated under reduced pressure to provide a residue that was purified by preparative RP-HPLC (water/acetonitrile with 0.1% formic acid in each). Combined and lyophilized pure product containing fractions to afford the title compound (60 mg, 50.12 μmol, 14.20% yield, 90.0% purity). MS (ESI) m/z: 1077.1 [M+H]⁺. ¹H NMR (400 MHz, DMSO-d6) δ 12.86-12.49 (m, 1H), 8.98 (s, 1H), 8.35 (d, J=7.6 Hz, 1H), 8.03 (d, J=7.6 Hz, 1H), 8.00-7.97 (m, 1H), 7.86-7.72 (m, 2H), 7.62 (d, J=7.2 Hz, 1H), 7.49-7.30 (m, 10H), 7.12-7.06 (m, 1H), 6.96 (d, J=8.4 Hz, 1H), 6.89-6.85 (m, 1H), 6.63 (d, J=6.8 Hz, 1H), 4.98 (s, 2H), 4.94-4.88 (m, 1H), 4.51 (d, J=9.6 Hz, 1H), 4.45-4.35 (m, 1H), 4.24 (s, 1H), 3.99-3.88 (m, 4H), 3.66-3.50 (m, 2H), 3.03 (t, J=5.6 Hz, 2H), 2.67 (s, 1H), 2.29 (d, J=1.2 Hz, 2H), 2.15 (s, 1H), 2.06-1.95 (m, 2H), 1.89 (s, 3H), 1.78-1.70 (m, 3H), 1.64-1.50 (m, 3H), 1.43 (d, J=8.0 Hz, 2H), 1.37 (d, J=6.8 Hz, 3H), 0.92 (s, 9H).

### Example 2. Preparation of Compound 10001a

### 2-(5-(1-((3r,5r,7r)-Adamantan-1-ylmethyl)-5-methyl-1H-pyrazol-4-yl)-6-((10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecyl)carbamoyl)pyridin-2-yl)-N-(benzo[d]thiazol-2-yl)-1,2,3,4-tetrahydroisoquinoline-8-carboxamide

### Step A. Methyl 10-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)decanoate

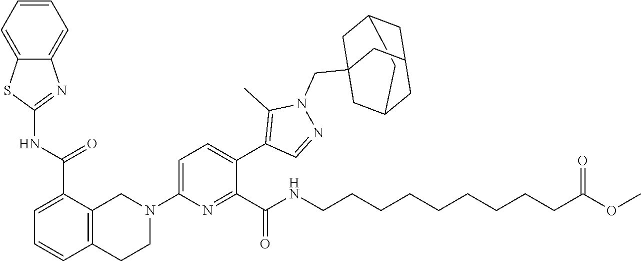 [SMILES: COC(=O)CCCCCCCCCNC(=O)c1nc(N2CCc3cccc(C(=O)Nc4nc5ccccc5s4)c3C2)ccc1-c1cnn(CC23CC4CC(CC(C4)C2)C3)c1C]

To a solution of 3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinic acid (1 g, 1.52 mmol, 1 equiv.) in DMF (10 mL) was added methyl 10-aminodecanoate (360.90 mg, 1.52 mmol, 1 equiv., HCl), HATU (577.15 mg, 1.52 mmol, 1 equiv.) and DIPEA (588.53 mg, 4.55 mmol, 793.17 μL, 3 equiv.) at 25° C. The reaction mixture was stirred at 25° C. for 1.5 h and the diluted with H₂O (10 mL) and extracted with EtOAc (30 mL×3). The combined organic layers were washed with a saturated aqueous solution of NaHCO₃ (10 mL×3), brine (10 mL×3), dried over anhydrous sodium sulfate, filtered, and concentrated under reduced pressure to give a residue that was purified by silica gel chromatography (heptane/ethyl acetate gradient) to afford the title compound (861 mg, 989.73 μmol, 65.20% yield). MS (ESI) m/z: 842.9 [M+H]⁺.

### Step B. 10-(3-(1-(Adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)decanoic acid

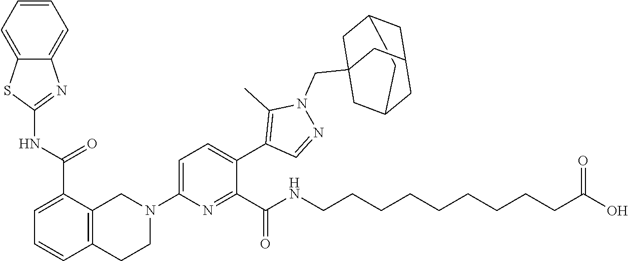 [SMILES: Cc1c(-c2ccc(N3CCc4cccc(C(=O)Nc5nc6ccccc6s5)c4C3)nc2C(=O)NCCCCCCCCCC(=O)O)cnn1CC12CC3CC(CC(C3)C1)C2]

To a solution of methyl 10-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)decanoate (831 mg, 986.82 μmol, 1 equiv.) in THF (10 mL) and H₂O (1 mL) was added LiOH·H₂O (124.23 mg, 2.96 mmol, 3 equiv.). The reaction mixture was stirred at 40° C. for 20 h and then concentrated under reduced pressure to give a residue. The residue was suspended in water (5 mL) and the pH was adjusted to ˜4 with HCl (aq.). The resulting solids were filtered, rinsed with water, and dried to afford the title compound (670 mg, 778.60 μmol, 78.90% yield). MS (ESI) m/z: 828.8 [M+H]⁺.

### Step C. 2-(5-(1-((3r,5r,7r)-adamantan-1-ylmethyl)-5-methyl-1H-pyrazol-4-yl)-6-((10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecyl)carbamoyl)pyridin-2-yl)-N-(benzo[d]thiazol-2-yl)-1,2,3,4-tetrahydroisoquinoline-8-carboxamide

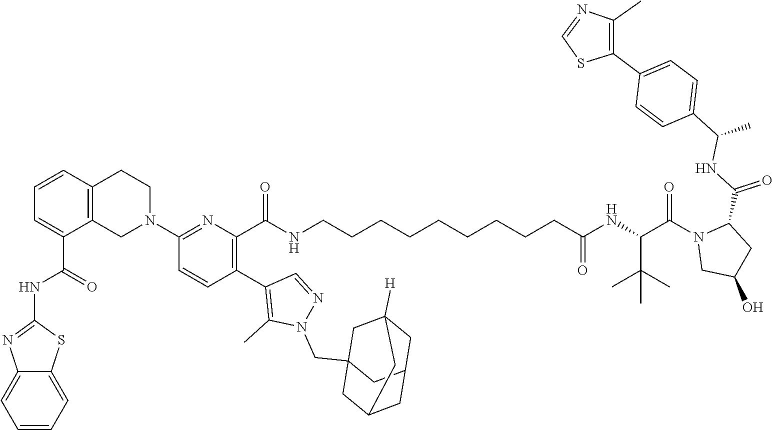 [SMILES: Cc1ncsc1-c1ccc([C@H](C)NC(=O)[C@@H]2C[C@@H](O)CN2C(=O)[C@@H](NC(=O)CCCCCCCCCNC(=O)c2nc(N3CCc4cccc(C(=O)Nc5nc6ccccc6s5)c4C3)ccc2-c2cnn(CC34CC5CC(CC(C5)C3)C4)c2C)C(C)(C)C)cc1]

To a solution of 10-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)decanoic acid (100.00 mg, 120.76 μmol, 1 equiv.) in DMF (1 mL) was added (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (53.69 mg, 120.76 μmol, 1 equiv.), HATU (45.92 mg, 120.76 μmol, 1 equiv.) and DIPEA (46.82 mg, 362.29 μmol, 63.10 μL, 3 equiv.). The reaction mixture was stirred at 25° C. for 16 h. and then purified by preparative RP-HPLC (water/acetonitrile with 0.1% formic acid in each). Combined and lyophilized pure product containing fractions to afford the title compound (32.33 mg, 29.18 μmol, 19.41% yield, 94.60% purity). MS (ESI) m/z: 1254.4 [M+H]⁺; ¹H NMR (400 MHz, DMSO-d₆) δ 12.96-12.73 (m, 1H), 8.98 (s, 1H), 8.36 (d, J=8.0 Hz, 1H), 8.09-7.96 (m, 2H), 7.82-7.72 (m, 2H), 7.60 (d, J=7.2 Hz, 1H), 7.48-7.32 (m, 7H), 7.23 (s, 1H), 6.95 (d, J=8.8 Hz, 1H), 5.09 (d, J=3.2 Hz, 1H), 5.00 (s, 2H), 4.95-4.89 (m, 1H), 4.52 (d, J=9.2 Hz, 1H), 4.42 (t, J=8.0 Hz, 1H), 4.28 (s, 1H), 3.88 (t, J=5.2 Hz, 2H), 3.67 (s, 2H), 3.60 (s, 1H), 3.01 (t, J=5.6 Hz, 4H), 2.45 (s, 3H), 2.23 (d, J=7.2 Hz, 1H), 2.13-2.08 (m, 1H), 2.05 (s, 3H), 1.99 (d, J=8.8 Hz, 1H), 1.91 (s, 3H), 1.80 (d, J=4.4 Hz, 1H), 1.67-1.55 (m, 6H), 1.52 (s, 6H), 1.47-1.40 (m, 2H), 1.37 (d, J=6.8 Hz, 3H), 1.30-1.23 (m, 3H), 1.20-1.06 (m, 10H), 0.93 (s, 9H).

### Example 3. Preparation of Compound 102a

### 6-[8-(1,3-Benzothiazol-2-ylcarbamoyl)-3,4-dihydro-1H-isoquinolin-2-yl]-3-[1-[16-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-16-oxo-hexadecyl]-3,5-dimethyl-pyrazol-4-yl]pyridine-2-carboxylic acid

### Step A. Methyl 16-(4-bromo-3,5-dimethyl-pyrazol-1-yl)hexadecanoate

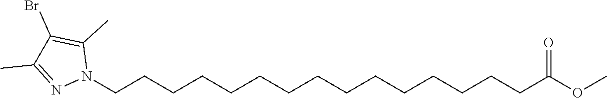

A mixture of methyl 16-bromohexadecanoate (997.98 mg, 2.86 mmol, 1 equiv.), 4-bromo-3,5-dimethyl-1H-pyrazole (500 mg, 2.86 mmol, 1 equiv.), K₂CO₃ (1.18 g, 8.57 mmol, 3 equiv.), Cs₂CO₃ (930.77 mg, 2.86 mmol, 1 equiv.) and KI (47.42 mg, 285.67 μmol, 0.1 equiv.) in DMF (5 mL) was degassed and purged with nitrogen 3 times. The mixture was then stirred at 100° C. for 12 h under a nitrogen atmosphere. The reaction mixture was diluted with water (50 mL) and extracted with EtOAc (50 mL×3). The combined organic layers were washed with brine (50 mL×3), dried over anhydrous sodium sulfate, filtered, and concentrated under reduced pressure to give a residue. The residue was purified by silica gel chromatography (ethyl acetate/petroleum ether gradient) to afford the title compound (1 g, 2.24 mmol, 78.57% yield). MS (ESI) m/z: 445.1 [M+H]⁺.

### Step B. tert-Butyl 6-[8-(1,3-benzothiazol-2-ylcarbamoyl)-3,4-dihydro-1H-isoquinolin-2-yl]-3-[1-(16-methoxy-16-oxo-hexadecyl)-3,5-dimethyl-pyrazol-4-yl]pyridine-2-carboxylate

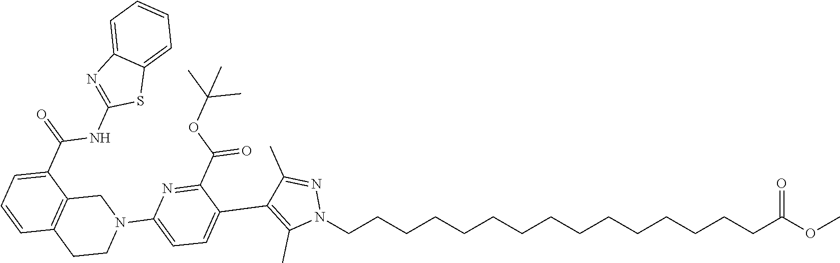 [SMILES: COC(=O)CCCCCCCCCCCCCCCn1nc(C)c(-c2ccc(N3CCc4cccc(C(=O)Nc5nc6ccccc6s5)c4C3)nc2C(=O)OC(C)(C)C)c1C]

A mixture of methyl 16-(4-bromo-3,5-dimethyl-pyrazol-1-yl)hexadecanoate (542.97 mg, 1.22 mmol, 1.5 eq), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (500 mg, 816.27 μmol, 1 equiv.), K₃PO₄ (1.5 M, 2.07 mL, 3.8 equiv.) and CataCxium Pd G3 (148.62 mg, 204.07 μmol, 0.25 equiv.) in DCE (2 mL) and 1,4-dioxane (5 mL) was degassed and purged with nitrogen 3 times. The mixture was then stirred at 100° C. for 12 hours under a nitrogen atmosphere. The reaction mixture was diluted with water (50 mL) and extracted with EtOAc (50 mL×3). The combined organic layers were washed with water (50 mL×3), dried over anhydrous sodium sulfate, filtered, and concentrated under reduced pressure to give a residue. The residue was purified by silica gel chromatography (petroleum ether/ethyl acetate gradient) to afford the title compound (500 mg, 567.64 μmol, 69.54% yield). MS (ESI) m/z: 849.4 [M+H]⁺.

### Step C. 16-(4-(6-(8-(Benzo[d]thiazol-2-ylcarbamoyl)-3,4-dihydroisoquinolin-2(1H)-yl)-2-(tert-butoxycarbonyl)pyridin-3-yl)-3,5-dimethyl-1H-pyrazol-1-yl)hexadecanoic acid

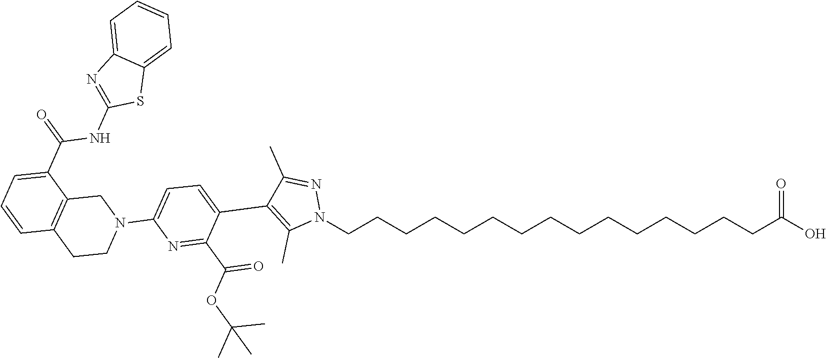 [SMILES: Cc1nn(CCCCCCCCCCCCCCCC(=O)O)c(C)c1-c1ccc(N2CCc3cccc(C(=O)Nc4nc5ccccc5s4)c3C2)nc1C(=O)OC(C)(C)C]

A mixture of tert-butyl 6-[8-(1,3-benzothiazol-2-ylcarbamoyl)-3,4-dihydro-1H-isoquinolin-2-yl]-3-[1-(16-methoxy-16-oxo-hexadecyl)-3,5-dimethyl-pyrazol-4-yl]pyridine-2-carboxylate (200 mg, 235.53 μmol, 1 equiv.) and LiOH (16.92 mg, 706.60 μmol, 3 equiv.) in THF (1.6 mL), H₂O (0.8 mL) and MeOH (0.4 mL) was stirred at 50° C. for 2 hours. The reaction mixture was diluted with water (20 mL) and extracted with EtOAc (20 mL×3). The combined organic layers were washed with brine (20 mL×3), dried over anhydrous sodium sulfate, filtered, and concentrated under reduced pressure to afford the title compound that was used without further purification (200 mg, 183.45 μmol, 77.89% yield). MS (ESI) m/z: 835.5 [M+H]⁺.

### Step D. tert-Butyl 6-[8-(1,3-benzothiazol-2-ylcarbamoyl)-3,4-dihydro-1H-isoquinolin-2-yl]-3-[1-[16-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-16-oxo-hexadecyl]-3,5-dimethyl-pyrazol-4-yl]pyridine-2-carboxylate

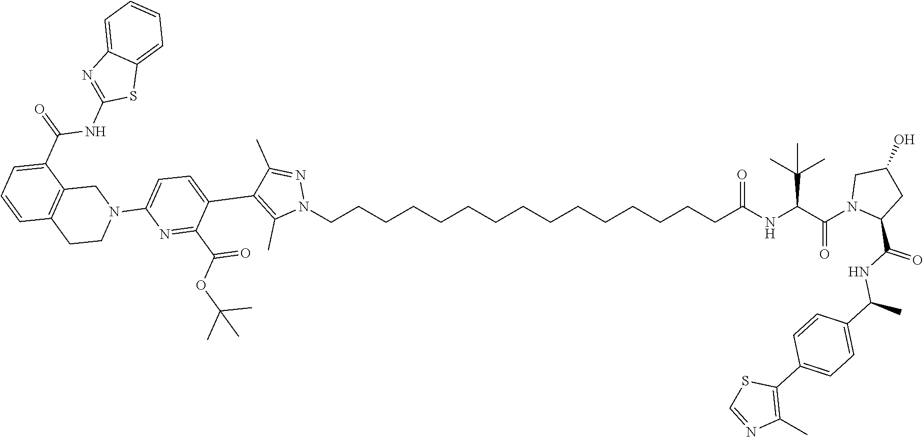 [SMILES: Cc1ncsc1-c1ccc([C@H](C)NC(=O)[C@@H]2C[C@@H](O)CN2C(=O)[C@@H](NC(=O)CCCCCCCCCCCCCCCn2nc(C)c(-c3ccc(N4CCc5cccc(C(=O)Nc6nc7ccccc7s6)c5C4)nc3C(=O)OC(C)(C)C)c2C)C(C)(C)C)cc1]

A mixture of 16-[4-[6-[8-(1,3-benzothiazol-2-ylcarbamoyl)-3,4-dihydro-1H-isoquinolin-2-yl]-2-tert-butoxycarbonyl-3-pyridyl]-3,5-dimethyl-pyrazol-1-yl]hexadecanoic acid (150 mg, 179.62 μmol, 1 equiv.), (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (95.83 mg, 215.54 μmol, 1.2 equiv.), HATU (102.44 mg, 269.43 μmol, 1.5 equiv.) and triethylamine (54.53 mg, 538.85 μmol, 75.00 μL, 3 equiv.) in DMF (1.5 mL) was stirred at 25° C. for 12 h. The reaction mixture was diluted with water 50 mL and extracted with EtOAc (50 mL×3). The combined organic layers were washed with brine (50 mL×3), dried over anhydrous sodium sulfate, filtered, and concentrated under reduced pressure to afford the title compound that was used without further purification (200 mg, 118.26 μmol, 65.84% yield). MS (ESI) m/z: 1261.6 [M+H]⁺.

### Step E. 6-[8-(1,3-Benzothiazol-2-ylcarbamoyl)-3,4-dihydro-1H-isoquinolin-2-yl]-3-[1-[16-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-16-oxo-hexadecyl]-3,5-dimethyl-pyrazol-4-yl]pyridine-2-carboxylic acid

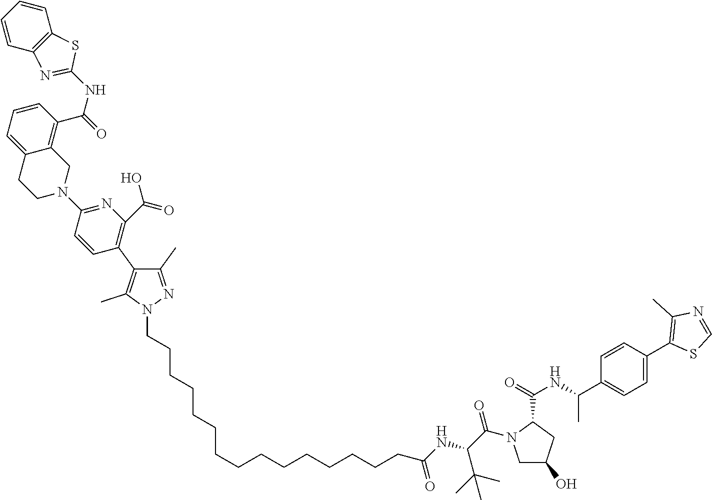 [SMILES: Cc1ncsc1-c1ccc([C@H](C)NC(=O)[C@@H]2C[C@@H](O)CN2C(=O)[C@@H](NC(=O)CCCCCCCCCCCCCCCn2nc(C)c(-c3ccc(N4CCc5cccc(C(=O)Nc6nc7ccccc7s6)c5C4)nc3C(=O)O)c2C)C(C)(C)C)cc1]

A mixture of tert-butyl 6-[8-(1,3-benzothiazol-2-ylcarbamoyl)-3,4-dihydro-1H-isoquinolin-2-yl]-3-[1-[16-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-16-oxo-hexadecyl]-3,5-dimethyl-pyrazol-4-yl]pyridine-2-carboxylate (100 mg, 79.26 μmol, 1 equiv.) in TFA (3.08 g, 27.01 mmol, 2.00 mL, 340.81 equiv.) and Tf₂O (2.24 mg, 7.93 μmol, 1.31 μL, 0.1 equiv.) was stirred at 25° C. for 0.5 h. The reaction mixture was concentrated to dryness under reduced pressure and purified by preparative RP-HPLC (water/acetonitrile, 0.1% formic acid in each). Combined and lyophilized pure product containing fractions to afford the title compound (5.59 mg, 4.29 μmol, 5.42% yield, 92.6% purity). MS (ESI) m/z: 1205.6 [M+H]⁺; ¹H NMR (400 MHz, CDCl3) δ=8.68 (s, 1H), 7.88 (d, J=8.4 Hz, 1H), 7.58 (d, J=7.6 Hz, 1H), 7.47-7.39 (m, 6H), 7.39-7.35 (m, 3H), 7.31-7.30 (m, 1H), 7.04 (d, J=8.4 Hz, 1H), 6.38-6.29 (m, 1H), 5.15 (s, 2H), 5.09 (t, J=7.2 Hz, 1H), 4.71-4.64 (m, 1H), 4.57-4.50 (m, 2H), 4.20-4.15 (m, 1H), 3.99 (t, J=7.2 Hz, 2H), 3.93 (t, J=6.0 Hz, 2H), 3.61-3.56 (m, 1H), 3.49-3.41 (m, 1H), 3.13 (t, J=6.0 Hz, 2H), 2.63-2.55 (m, 1H), 2.52 (s, 3H), 2.14 (t, J=7.6 Hz, 2H), 2.07 (s, 3H), 2.03 (s, 3H), 1.84-1.79 (m, 2H), 1.47 (d, J=6.8 Hz, 4H), 1.31 (s, 4H), 1.22 (s, 20H), 1.06 (s, 9H).

### Example 4. Preparation of Compound 101a

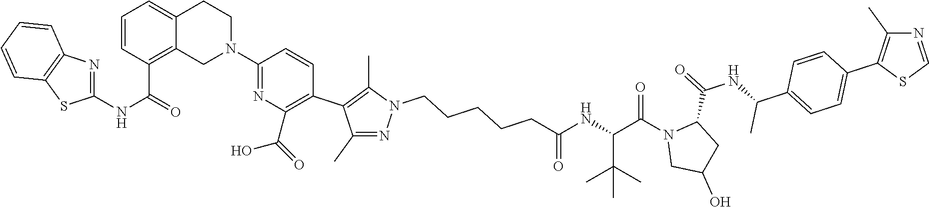 [SMILES: Cc1ncsc1-c1ccc([C@H](C)NC(=O)[C@@H]2CC(O)CN2C(=O)[C@@H](NC(=O)CCCCCn2nc(C)c(-c3ccc(N4CCc5cccc(C(=O)Nc6nc7ccccc7s6)c5C4)nc3C(=O)O)c2C)C(C)(C)C)cc1]

### 6-[8-(1,3-benzothiazol-2-ylcarbamoyl)-3,4-dihydro-1H-isoquinolin-2-yl]-3-[1-[6-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-6-oxo-hexyl]-3,5-dimethyl-pyrazol-4-yl]pyridine-2-carboxylic acid

### Step A. Procedure for Preparation of methyl 6-(4-bromo-3,5-dimethyl-pyrazol-1-yl)hexanoate

A mixture of 4-bromo-3,5-dimethyl-1H-pyrazole (1 g, 5.71 mmol, 1 equiv.), methyl 6-bromohexanoate (2.39 g, 11.43 mmol, 2 equiv.), K₂CO₃ (789.63 mg, 5.71 mmol, 1 equiv.) and KI (47.42 mg, 285.67 μmol, 0.05 equiv.) in DMF (10 mL) was degassed and purged with N₂ three times, and then the mixture was stirred at 100° C. for 12 hours under N₂ atmosphere. The reaction mixture was diluted with water (100 mL) and extracted with EtOAc (100 mL×3). The combined organic layers were then washed with brine (100 mL×3), dried over sodium sulfate, filtered, and concentrated under reduced pressure to give a residue. The residue was purified by column chromatography (SiO₂, petroleum ether: ethyl acetate=1:0 to 0:1). The compound methyl 6-(4-bromo-3,5-dimethyl-pyrazol-1-yl)hexanoate (700 mg, 2.1 mmol, 40.4% yield) was obtained as a yellow oil.

MS (ESI) m/z: 303.0 [M+H]⁺

¹H NMR (400 MHz, CDCl₃) δ=4.05-3.95 (m, 2H), 3.68 (s, 3H), 2.32 (t, J=7.6 Hz, 2H), 2.27-2.15 (m, 6H), 1.80 (q, J=7.6 Hz, 2H), 1.71-1.64 (m, 2H), 1.39-1.30 (m, 2H).

### Step B. Procedure for Preparation of tert-butyl 6-(8-(benzo[d]thiazol-2-ylcarbamoyl)-3,4-dihydroisoquinolin-2(1H)-yl)-3-(1-(6-methoxy-6-oxohexyl)-3,5-dimethyl-1H-pyrazol-4-yl)picolinate

A mixture of methyl 6-(4-bromo-3,5-dimethyl-pyrazol-1-yl)hexanoate (371.23 mg, 1.22 mmol, 1.5 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (500 mg, 816.27 μmol, 1 equiv.), K₃PO₄ (1.5 M, 2.07 mL, 3.8 equiv.), and [2-(2-aminophenyl)phenyl]palladium(1+);bis(1-adamantyl)-butyl-phosphane;methanesulfonate (148.62 mg, 204.07 μmol, 0.25 equiv.) in DCE (2 mL) and 1,4-dioxane (10 mL) was degassed and purged with N₂ three times, and then the mixture was stirred at 100° C. for 12 hours under N₂ atmosphere. The reaction mixture was diluted with water (100 mL) and extracted with EtOAc (100 mL×3) then the combined organic layers were washed with brine (100 mL×3), dried over sodium sulfate, filtered, and concentrated under reduced pressure to give a residue. The residue was purified by column chromatography (SiO₂, petroleum ether/ethyl acetate=1/0 to 3/2). tert-Butyl 6-(8-(benzo[d]thiazol-2-ylcarbamoyl)-3,4-dihydroisoquinolin-2(1H)-yl)-3-(1-(6-methoxy-6-oxohexyl)-3,5-dimethyl-1H-pyrazol-4-yl)picolinate (250 mg, 336.1 μmol, 41.1% yield, 95.3% purity) was obtained as a yellow solid.

MS (ESI) m/z: 709.4 [M+H]⁺

¹H NMR (400 MHz, CDCl₃) δ=7.83-7.72 (m, 1H), 7.47 (d, J=7.6 Hz, 1H), 7.34-7.32 (m, 1H), 7.29-7.21 (m, 4H), 7.18-7.12 (m, 1H), 6.79 (d, J=8.8 Hz, 1H), 4.95 (s, 2H), 4.03-3.95 (m, 2H), 3.89 (t, J=7.6 Hz, 2H), 3.59 (s, 3H), 2.98 (t, J=5.6 Hz, 2H), 2.25 (t, J=7.6 Hz, 2H), 1.95 (d, J=10.8 Hz, 6H), 1.79-1.71 (m, 2H), 1.59 (d, J=7.6 Hz, 2H), 1.37-1.30 (m, 2H), 1.21-1.18 (m, 9H).

### Step C. Procedure for Preparation of 6-[4-[6-[8-(1,3-benzothiazol-2-ylcarbamoyl)-3,4-dihydro-1H-isoquinolin-2-yl]-2-tert-butoxycarbonyl-3-pyridyl]-3,5-dimethyl-pyrazol-1-yl]hexanoic acid

To a solution of tert-butyl 6-(8-(benzo[d]thiazol-2-ylcarbamoyl)-3,4-dihydroisoquinolin-2(1H)-yl)-3-(1-(6-methoxy-6-oxohexyl)-3,5-dimethyl-1H-pyrazol-4-yl)picolinate (200 mg, 282.14 μmol, 1 equiv.) in THF (1.5 mL) and water (0.7 mL) was added LiOH (20.27 mg, 846.42 μmol, 3 equiv.). The mixture was stirred at 25° C. for 0.5 hours. The reaction mixture was diluted with water (30 mL) and extracted with EtOAc (30 mL×3). The combined organic layers were washed with brine (30 mL×3), dried over sodium sulfate, filtered, and concentrated under reduced pressure to give a residue. The residue was purified by prep-TLC (SiO₂, DCM:MeOH=10:1). The compound 6-[4-[6-[8-(1,3-benzothiazol-2-ylcarbamoyl)-3,4-dihydro-1H-isoquinolin-2-yl]-2-tert-butoxycarbonyl-3-pyridyl]-3,5-dimethyl-pyrazol-1-yl]hexanoic acid (200 mg, 261.6 μmol, 92.7% yield, 90.9% purity) was obtained as a yellow solid.

MS (ESI) m/z: 695.2 [M+H]⁺

### Step D. Procedure for Preparation of tert-butyl 6-[8-(1,3-benzothiazol-2-ylcarbamoyl)-3,4-dihydro-1H-isoquinolin-2-yl]-3-[1-[6-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-6-oxo-hexyl]-3,5-dimethyl-pyrazol-4-yl]pyridine-2-carboxylate

A mixture of 6-[4-[6-[8-(1,3-benzothiazol-2-ylcarbamoyl)-3,4-dihydro-1H-isoquinolin-2-yl]-2-tert-butoxycarbonyl-3-pyridyl]-3,5-dimethyl-pyrazol-1-yl]hexanoic acid (100 mg, 143.92 μmol, 1 equiv.), (2S,4R)-1-[(2S)-2-amino-3,3-dimethyl-butanoyl]-4-hydroxy-N-[(iS)-1-[4-(4-methylthiazol-5-yl)phenyl]ethyl]pyrrolidine-2-carboxamide (76.78 mg, 172.70 μmol, 1.2 equiv.), HATU (82.08 mg, 215.88 μmol, 1.5 equiv.), and Et₃N (43.69 mg, 431.75 μmol, 60.09 μL, 3 equiv.) in DMF (1 mL) was degassed and purged with N₂ three times, and then the mixture was stirred at 25° C. for 12 hours under N₂ atmosphere. The reaction mixture was then diluted with water (30 mL) and extracted with EtOAc (30 mL×3). The combined organic layers were washed with water (30 mL×3), dried over sodium sulfate, filtered and concentrated under reduced pressure to give a residue. The residue was purified by prep-TLC (SiO₂, DCM:MeOH=10:1). tert-Butyl 6-[8-(1,3-benzothiazol-2-ylcarbamoyl)-3,4-dihydro-1H-isoquinolin-2-yl]-3-[1-[6-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-6-oxo-hexyl]-3,5-dimethyl-pyrazol-4-yl]pyridine-2-carboxylate (100 mg, 62.0 μmol, 43.1% yield, 69.6% purity) was obtained as a white solid.

MS (ESI) m/z: 1121.7 [M+H]⁺

¹H NMR (400 MHz, CDCl₃) δ=8.60 (s, 1H), 7.81-7.73 (m, 1H), 7.50 (d, J=7.6 Hz, 1H), 7.34-7.14 (m, 9H), 6.81-6.74 (m, 1H), 6.42-6.27 (m, 1H), 4.99-4.91 (m, 2H), 4.69-4.61 (m, 1H), 4.50-4.38 (m, 6H), 4.03-3.94 (m, 2H), 3.91-3.82 (m, 2H), 3.56-3.45 (m, 1H), 3.03-2.92 (m, 2H), 2.34-2.26 (m, 1H), 2.28-2.22 (m, 3H), 1.95-1.88 (m, 6H), 1.75-1.52 (m, 6H), 1.42-1.34 (m, 3H), 1.18 (s, 9H), 0.96 (s, 9H).

### Step E. Procedure for Preparation of 6-[8-(1,3-benzothiazol-2-ylcarbamoyl)-3,4-dihydro-1H-isoquinolin-2-yl]-3-[1-[6-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-6-oxo-hexyl]-3,5-dimethyl-pyrazol-4-yl]pyridine-2-carboxylic acid

A mixture of tert-butyl 6-[8-(1,3-benzothiazol-2-ylcarbamoyl)-3,4-dihydro-1H-isoquinolin-2-yl]-3-[1-[6-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-6-oxo-hexyl]-3,5-dimethyl-pyrazol-4-yl]pyridine-2-carboxylate (80 mg, 71.34 μmol, 1 equiv.) and TFA (1.23 g, 10.81 mmol, 800.00 μL, 151.46 equiv.) in CH₂Cl₂ (800 μL) was degassed and purged with N₂ and stirred at 25° C. for 2 hours under N₂ atmosphere. The reaction mixture was filtered and concentrated under reduced pressure to give a residue. The residue was purified by prep-HPLC. 6-[8-(1,3-Benzothiazol-2-ylcarbamoyl)-3,4-dihydro-1H-isoquinolin-2-yl]-3-[1-[6-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-6-oxo-hexyl]-3,5-dimethyl-pyrazol-4-yl]pyridine-2-carboxylic acid (4.2 mg, 3.8 μmol, 5.3% yield, 95.7% purity) was obtained as a white solid MS (ESI) m/z: 1065.6 [M+H]⁺

¹H NMR (400 MHz, CD₃OD) δ=8.86 (s, 1H), 7.91 (d, J=8.0 Hz, 1H), 7.77 (d, J=7.6 Hz, 1H), 7.59 (d, J=7.2 Hz, 1H), 7.49-7.25 (m, 9H), 7.01 (d, J=9.2 Hz, 1H), 5.06 (s, 3H), 5.01-4.96 (m, 2H), 4.62-4.53 (m, 2H), 4.41 (s, 1H), 3.99 (d, J=5.6 Hz, 4H), 3.87 (d, J=11.2 Hz, 1H), 3.76-3.70 (m, 1H), 3.09 (s, 2H), 2.49-2.41 (m, 3H), 2.32-2.24 (m, 2H), 2.21-2.15 (m, 1H), 2.04 (d, J=18.0 Hz, 6H), 1.84-1.75 (m, 2H), 1.67-1.53 (m, 3H), 1.49 (br d, J=6.8 Hz, 3H), 1.36-1.28 (m, 3H), 1.07-0.93 (m, 9H)

### Example 5. Preparation of Compound 103a

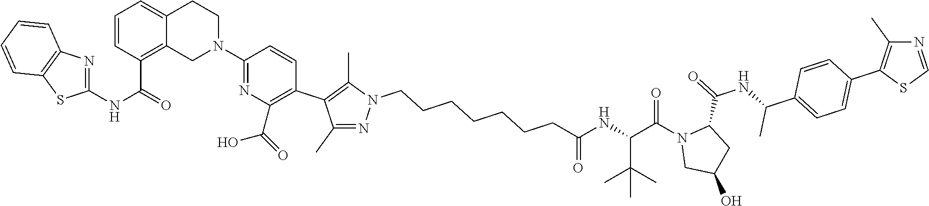 [SMILES: Cc1ncsc1-c1ccc([C@H](C)NC(=O)[C@@H]2C[C@@H](O)CN2C(=O)[C@@H](NC(=O)CCCCCCCn2nc(C)c(-c3ccc(N4CCc5cccc(C(=O)Nc6nc7ccccc7s6)c5C4)nc3C(=O)O)c2C)C(C)(C)C)cc1]

### 6-[8-(1,3-benzothiazol-2-ylcarbamoyl)-3,4-dihydro-1H-isoquinolin-2-yl]-3-[1-[8-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-8-oxo-octyl]-3,5-dimethyl-pyrazol-4-yl]pyridine-2-carboxylic acid

### Step A. Procedure for Preparation of methyl 8-bromooctanoate

To a solution of 8-bromooctanoic acid (10 g, 44.82 mmol, 1 equiv.) in MeOH (50 mL) was added SOCl₂ (6.40 g, 53.79 mmol, 3.90 mL, 1.2 equiv.) at 0° C. The mixture was stirred at 65° C. for 12 hours. The reaction mixture was concentrated under reduced pressure to give a residue. The crude product was used to the next reaction. Methyl 8-bromooctanoate (13 g, crude) was obtained as a brown liquid.

¹H NMR (400 MHz, CD₃OD) δ=3.58 (s, 3H), 3.50-3.46 (m, 1H), 3.38-3.31 (m, 1H), 2.27-2.23 (m, 2H), 1.79-1.67 (m, 2H), 1.54 (s, 2H), 1.38 (s, 2H), 1.27 (s, 4H).

### Step B. Procedure for Preparation of methyl 8-(4-bromo-3,5-dimethyl-pyrazol-1-yl)octanoate

To a solution of methyl 8-bromooctanoate (5 g, 21.09 mmol, 1 equiv.) and 4-bromo-3,5-dimethyl-1H-pyrazole (3.69 g, 21.09 mmol, 1 equiv.) in DMF (30 mL) was added K₂CO₃ (8.74 g, 63.26 mmol, 3 equiv.) and KI (175.01 mg, 1.05 mmol, 0.05 equiv.). The mixture was stirred at 90° C. for 9 hours. The residue was washed with brine (90 mL) and extracted with DCM 90 mL (30 mL×3), dried over anhydrous sodium sulfate, filtered, and concentrated under reduced pressure to give a residue. The residue was purified by flash silica gel chromatography (eluent of 0˜25% ethyl acetate/petroleum ether). The compound methyl 8-(4-bromo-3,5-dimethyl-pyrazol-1-yl)octanoate (2.2 g, 6.8 mmol, 32.3% yield) was obtained as a yellow oil.

MS (ESI) m/z: 332.8 [M+H]⁺.

¹H NMR (400 MHz, CDCl₃) δ=3.95-3.91 (m, 2H), 3.63 (s, 3H), 2.28-2.24 (m, 2H), 2.19-2.16 (m, 6H), 1.74-1.70 (m, 2H), 1.59-1.56 (m, 2H), 1.29 (s, 6H).

### Step C. Procedure for Preparation of tert-butyl 6-[8-(1,3-benzothiazol-2-ylcarbamoyl)-3,4-dihydro-1H-isoquinolin-2-yl]-3-[1-(8-methoxy-8-oxo-octyl)-3,5-dimethyl-pyrazol-4-yl]pyridine-2-carboxylate

To a mixture of methyl 8-(4-bromo-3,5-dimethyl-pyrazol-1-yl)octanoate (494.81 mg, 1.49 mmol, 1.5 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (610 mg, 995.84 μmol, 1 equiv.), and K₃PO₄ (1.5 M, 2.52 mL, 3.8 equiv.) in 1,4-dioxane (10 mL), was added [2-(2-aminophenyl)phenyl]palladium(1+);bis(1-adamantyl)-butyl-phosphane;methanesulfonate (181.31 mg, 248.96 μmol, 0.25 equiv.) and DCE (2 mL). The reaction mixture was degassed and purged with N₂ three times, and then the mixture was stirred at 100° C. for 9 hours under N₂ atmosphere. The reaction mixture was concentrated under reduced pressure to give a residue. The residue was purified by column chromatography (SiO₂, petroleum ether/ethyl acetate=1/0 to 2/1). tert-Butyl 6-[8-(1,3-benzothiazol-2-ylcarbamoyl)-3,4-dihydro-1H-isoquinolin-2-yl]-3-[1-(8-methoxy-8-oxo-octyl)-3,5-dimethyl-pyrazol-4-yl]pyridine-2-carboxylate (355 mg, 481.7 μmol, 48.3% yield) was obtained as a yellow solid.

MS (ESI) m/z: 738.5 [M+H]⁺.

¹H NMR (400 MHz, CDCl₃) δ 7.83-7.72 (m, 1H), 7.51-7.44 (m, 1H), 7.40-7.32 (m, 1H), 7.30-7.21 (m, 4H), 7.17-7.09 (m, 1H), 6.79 (d, J=8.8 Hz, 1H), 5.03-4.88 (m, 2H), 4.03-3.96 (m, 2H), 3.87 (t, J=7.6 Hz, 2H), 3.58 (s, 3H), 3.01-2.97 (m, 2H), 2.23 (t, J=7.6 Hz, 2H), 1.98-1.90 (m, 6H), 1.70 (d, J=7.2 Hz, 2H), 1.56 (s, 5H), 1.27 (s, 4H), 1.23-1.17 (m, 9H)

### Step D. Procedure for Preparation of 8-[4-[6-[8-(1,3-benzothiazol-2-ylcarbamoyl)-3,4-dihydro-1H-isoquinolin-2-yl]-2-tert-butoxycarbonyl-3-pyridyl]-3,5-dimethyl-pyrazol-1-yl]octanoic acid

To a solution of tert-butyl 6-[8-(1,3-benzothiazol-2-ylcarbamoyl)-3,4-dihydro-1H-isoquinolin-2-yl]-3-[1-(8-methoxy-8-oxo-octyl)-3,5-dimethyl-pyrazol-4-yl]pyridine-2-carboxylate (200 mg, 271.40 μmol, 1 equiv.) in THF (10 mL) was added LiOH·H₂O (19.50 mg, 814.20 μmol, 3 equiv.), water (1 mL) and MeOH (1 mL). The mixture was stirred at 25° C. for 2 hours. The reaction mixture was concentrated under reduced pressure to give a residue. The residue was purified by prep-TLC (SiO₂, ethyl acetate/petroleum ether=2:1). 8-[4-[6-[8-(1,3-Benzothiazol-2-ylcarbamoyl)-3,4-dihydro-1H-isoquinolin-2-yl]-2-tert-butoxycarbonyl-3-pyridyl]-3,5-dimethyl-pyrazol-1-yl]octanoic acid (136 mg, 188.1 μmol, 69.3% yield) was obtained as a yellow solid.

MS (ESI) m/z: 723.4 [M+H]⁺.

¹H NMR (400 MHz, CDCl₃) δ=7.75-7.72 (m, 1H), 7.46 (d, J=7.2 Hz, 1H), 7.24-7.21 (m, 3H), 7.19 (s, 2H), 7.16-7.13 (m, 1H), 6.72 (d, J=8.8 Hz, 1H), 5.23 (s, 1H), 4.81-4.77 (m, 1H), 4.08-4.03 (m, 1H), 3.93-3.82 (m, 4H), 2.97 (t, J=5.2 Hz, 2H), 1.96 (d, J=12.8 Hz, 6H), 1.88 (s, 3H), 1.66 (s, 3H), 1.40-1.36 (m, 2H), 1.19-1.15 (m, 8H), 1.11 (s, 9H).

### Step E. Procedure for Preparation of tert-butyl 6-[8-(1,3-benzothiazol-2-ylcarbamoyl)-3,4-dihydro-1H-isoquinolin-2-yl]-3-[1-[8-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-8-oxo-octyl]-3,5-dimethyl-pyrazol-4-yl]pyridine-2-carboxylate

To a solution of 8-[4-[6-[8-(1,3-benzothiazol-2-ylcarbamoyl)-3,4-dihydro-1H-isoquinolin-2-yl]-2-tert-butoxycarbonyl-3-pyridyl]-3,5-dimethyl-pyrazol-1-yl]octanoic acid (100 mg, 138.33 μmol, 1 equiv.) in DMF (1 mL) was added HATU (78.90 mg, 207.50 μmol, 1.5 equiv.) and TEA (41.99 mg, 415.00 μmol, 57.76 μL, 3 equiv.). The mixture was stirred at 25° C. for 0.5 hours, and then(2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (73.80 mg, 166.00 μmol, 1.2 equiv.) was added to the mixture. The mixture was stirred at 25° C. for 8.5 hours. The reaction mixture was concentrated under reduced pressure to give a residue. The residue was purified by prep-HPLC. tert-Butyl 6-[8-(1,3-benzothiazol-2-ylcarbamoyl)-3,4-dihydro-1H-isoquinolin-2-yl]-3-[1-[8-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-8-oxo-octyl]-3,5-dimethyl-pyrazol-4-yl]pyridine-2-carboxylate (77 mg, 66.9 μmol, 48.4% yield) was obtained as a yellow solid.

MS (ESI) m/z: 575.7 [M+2H/2]⁺.

¹H NMR (400 MHz, CDCl₃) δ=8.68 (s, 1H), 7.86 (d, J=7.6 Hz, 1H), 7.69-7.67 (m, 1H), 7.58 (d, J=7.6 Hz, 1H), 7.40-7.31 (m, 10H), 6.86 (d, J=8.8 Hz, 1H), 6.19-6.15 (m, 1H), 5.09 (t, J=7.2 Hz, 1H), 5.04 (s, 2H), 4.77-4.73 (m, 1H), 4.59-4.52 (m, 2H), 4.16-4.04 (m, 5H), 3.95 (t, J=7.6 Hz, 2H), 3.60-3.56 (m, 1H), 3.07 (t, J=5.6 Hz, 2H), 2.52 (s, 3H), 2.30-2.15 (m, 3H), 2.05-2.02 (m, 9H), 1.77-1.61 (m, 11H), 1.47 (d, J=6.8 Hz, 3H), 1.27 (s, 9H), 1.04 (s, 9H).

### Step F. Procedure for Preparation of 6-[8-(1,3-benzothiazol-2-ylcarbamoyl)-3,4-dihydro-1H-isoquinolin-2-yl]-3-[1-[8-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-8-oxo-octyl]-3,5-dimethyl-pyrazol-4-yl]pyridine-2-carboxylic acid

To a solution of tert-butyl 6-[8-(1,3-benzothiazol-2-ylcarbamoyl)-3,4-dihydro-1H-isoquinolin-2-yl]-3-[1-[8-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-8-oxo-octyl]-3,5-dimethyl-pyrazol-4-yl]pyridine-2-carboxylate (50 mg, 43.50 μmol, 1 equiv.) in CH₂Cl₂ (0.5 mL) was added TFA (770.00 mg, 6.75 mmol, 0.5 mL, 155.25 equiv.). The mixture was stirred at 25° C. for 1 hour. The reaction mixture was concentrated under reduced pressure to give a residue. The residue was purified by prep-HPLC. 6-[8-(1,3-Benzothiazol-2-ylcarbamoyl)-3,4-dihydro-1H-isoquinolin-2-yl]-3-[1-[8-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-8-oxo-octyl]-3,5-dimethyl-pyrazol-4-yl]pyridine-2-carboxylic acid (23.4 mg, 21.1 μmol, 48.6% yield, 98.8% purity) was obtained as a white solid.

MS (ESI) m/z: 1094.4 [M+H]⁺.

¹H NMR (400 MHz, CDCl₃) δ=8.66 (d, J=4.0 Hz, 1H), 7.87-7.84 (m, 1H), 7.65-7.58 (m, 2H), 7.50-7.32 (m, 9H), 7.01 (d, J=8.8 Hz, 1H), 6.30-6.27 (m, 1H), 5.15 (s, 2H), 5.11-5.08 (m, 1H), 4.72-4.68 (m, 1H), 4.64-4.60 (m, 1H), 4.52-4.51 (m, 1H), 4.16-4.08 (m, 1H), 4.03-3.91 (m, 4H), 3.60-3.56 (m, 1H), 3.11 (t, J=6.0 Hz, 2H), 2.51-2.44 (m, 4H), 2.25-2.02 (m, 10H), 1.90-1.76 (m, 4H), 1.49 (d, J=6.8 Hz, 4H), 1.29 (s, 7H), 1.03 (s, 9H).

### Example 6. Preparation of Compound 104a

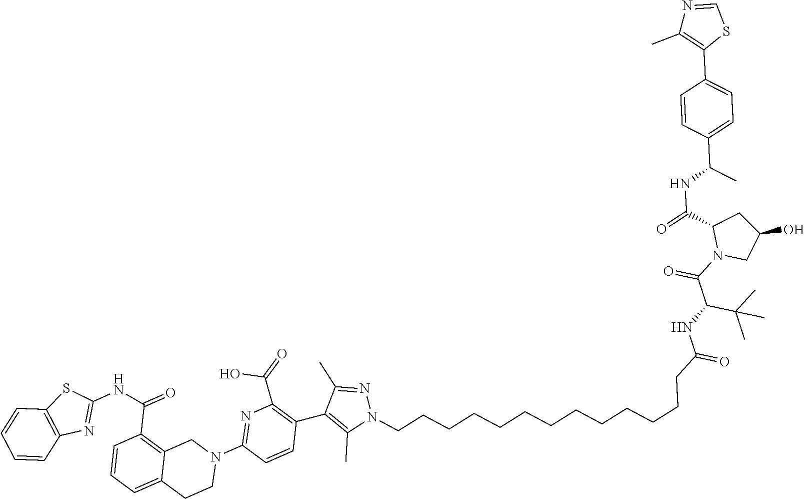 [SMILES: Cc1ncsc1-c1ccc([C@H](C)NC(=O)[C@@H]2C[C@@H](O)CN2C(=O)[C@@H](NC(=O)CCCCCCCCCCCCCn2nc(C)c(-c3ccc(N4CCc5cccc(C(=O)Nc6nc7ccccc7s6)c5C4)nc3C(=O)O)c2C)C(C)(C)C)cc1]

### 6-[8-(1,3-benzothiazol-2-ylcarbamoyl)-3,4-dihydro-1H-isoquinolin-2-yl]-3-[1-[14-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-14-oxo-tetradecyl]-3,5-dimethyl-pyrazol-4-yl]pyridine-2-carboxylic acid

### Step A. Procedure for Preparation of methyl 14-hydroxytetradecanoate

To a solution of 14-hydroxytetradecanoic acid (4 g, 16.37 mmol, 1 equiv.) in DMF (40 mL) was added K₂CO₃ (6.79 g, 49.11 mmol, 3 equiv.), then Mel (3.49 g, 24.55 mmol, 1.53 mL, 1.5 equiv.) was added, and the mixture was stirred at 80° C. for 4 hours under N₂ atmosphere. The reaction mixture was quenched by saturated NH₄Cl (6 mL) at 25° C., then extracted with ethyl acetate 45 mL (15 mL×3). The combined organic layers were washed with brine 18 mL (6 mL×3), dried over sodium sulfate, filtered, and concentrated under reduced pressure to give methyl 14-hydroxytetradecanoate (4.6 g, crude) as a white solid.

¹H NMR (400 MHz, CDCl₃) δ=3.67-3.53 (m, 3H), 3.05-2.76 (m, 2H), 2.45-2.05 (m, 2H), 1.63-1.41 (m, 4H), 1.20 (d, J=7.6 Hz, 10H), 0.80-0.42 (m, 8H)

### Step B. Procedure for Preparation of methyl 14-(p-tolylsulfonyloxy)tetradecanoate

A mixture of methyl 14-hydroxytetradecanoate (2.62 g, 10.14 mmol, 1 equiv.), DMAP (619.36 mg, 5.07 mmol, 0.5 equiv.), and TEA (3.08 g, 30.42 mmol, 4.23 mL, 3 equiv.) in DCM (26.2 mL) was degassed and purged with N₂ three times, then 4-methylbenzenesulfonyl chloride (3.87 g, 20.28 mmol, 2 equiv.) was added at 0° C. The mixture was stirred at 25° C. for 4 hours under N₂ atmosphere. The residue was diluted with water (25 mL) and extracted with DCM 75 mL (25 mL×3). The combined organic layers were washed with brine 60 mL (20 mL×3), dried over sodium sulfate, filtered, and concentrated under reduced pressure to give a residue. The residue was purified by column chromatography (SiO₂, petroleum ether/ethyl acetate=30/1 to 20/1) to give methyl 14-(p-tolylsulfonyloxy)tetradecanoate (3.3 g, 8.0 mmol, 79.8% yield) as a yellow oil.

¹H NMR (400 MHz, CDCl₃) δ=7.72 (d, J=8.4 Hz, 2H), 7.27 (d, J=8.0 Hz, 2H), 3.95 (t, J=6.4 Hz, 2H), 3.59 (s, 3H), 2.38 (s, 3H), 2.24-2.22 (s, 2H), 1.57-1.51 (m, 6H), 1.22-1.21 (m, 16H).

### Step C. Procedure for Preparation of methyl 14-bromotetradecanoate

To a solution of methyl 14-(p-tolylsulfonyloxy)tetradecanoate (2.7 g, 6.54 mmol, 1 equiv.) in acetone (50 mL) was added LiBr (5.68 g, 65.44 mmol, 1.64 mL, 10 equiv.). The mixture was stirred at 70° C. for 12 hours. The reaction mixture was filtered and concentrated under reduced pressure to remove the acetone, then extracted with ethyl acetate 60 mL (20 mL×3). The combined organic layers were dried over sodium sulfate, filtered, and concentrated under reduced pressure to give methyl 14-bromotetradecanoate (2 g, crude) as a brown oil.

¹H NMR (400 MHz, CDCl₃) δ=3.60 (s, 3H), 3.34 (t, J=6.8 Hz, 2H), 2.25-2.21 (m, 2H), 1.82-1.73 (m, 2H), 1.54 (d, J=7.2 Hz, 2H), 1.34 (d, J=7.6 Hz, 2H), 1.22-1.19 (m, 16H).

### Step D. Procedure for Preparation of methyl 14-(4-bromo-3,5-dimethyl-pyrazol-1-yl)tetradecanoate

To a solution of methyl 14-bromotetradecanoate (2 g, 6.22 mmol, 1 equiv.) and 4-bromo-3,5-dimethyl-1H-pyrazole (1.09 g, 6.22 mmol, 1 equiv.) in DMF (20 mL) was added K₂CO₃ (2.58 g, 18.67 mmol, 3 equiv.) and KI (51.67 mg, 311.24 μmol, 0.05 equiv.). The mixture was stirred at 90° C. for 6 hours. The reaction mixture was quenched by water (50 mL) and extracted with ethyl acetate 60 mL (20 mL×3). The combined organic layers were washed with brine 45 mL (15 mL×3), dried over sodium sulfate, filtered, and concentrated under reduced pressure to give a residue. The residue was purified by column chromatography (SiO₂, petroleum ether/ethyl acetate=1/0 to 0/1) to give methyl 14-(4-bromo-3,5-dimethyl-pyrazol-1-yl)tetradecanoate (1.35 g, 3.1 mmol, 50.9% yield, 97.5% purity) as a brown oil.

MS (ESI) m/z: 416.5 [M+H]⁺.

### Step E. Procedure for Preparation of tert-butyl 6-[8-(1,3-benzothiazol-2-ylcarbamoyl)-3,4-dihydro-1H-isoquinolin-2-yl]-3-[1-(14-methoxy-14-oxo-tetradecyl)-3,5-dimethyl-pyrazol-4-yl]pyridine-2-carboxylate

To a solution of methyl 14-(4-bromo-3,5-dimethyl-pyrazol-1-yl)tetradecanoate (0.5 g, 1.20 mmol, 1 equiv.) and tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (884.74 mg, 1.44 mmol, 1.2 equiv.) in 1,4-dioxane (8 mL) and water (2 mL) was added K₃PO4 (766.49 mg, 3.61 mmol, 3 equiv.) and [2-(2-aminophenyl)phenyl]palladium(1+);bis(1-adamantyl)-butyl-phosphane;methanesulfonate (87.66 mg, 120.36 μmol, 0.1 equiv.). The mixture was stirred at 100° C. for 9 hours to give a brown solution. The reaction mixture was filtered and concentrated under reduced pressure to give a residue. The residue was purified by prep-HPLC to provide tert-butyl 6-[8-(1,3-benzothiazol-2-ylcarbamoyl)-3,4-dihydro-1H-isoquinolin-2-yl]-3-[1-(14-methoxy-14-oxo-tetradecyl)-3,5-dimethyl-pyrazol-4-yl]pyridine-2-carboxylate (0.2 g, 238.7 μmol, 19.8% yield, 98% purity) as a brown oil.

MS (ESI) m/z: 821.3 [M+H]⁺.

¹H NMR (400 MHz, CDCl₃) δ=7.84 (d, J=7.6 Hz, 1H), 7.57-7.54 (m, 1H), 7.39-7.37 (m, 1H), 7.35-7.21 (m, 4H), 7.18-7.10 (m, 1H), 6.87 (d, J=8.8 Hz, 1H), 5.00 (s, 2H), 4.09-4.05 (m, 2H), 3.99-3.94 (m, 2H), 3.67 (s, 3H), 3.07-3.04 (m, 2H), 2.33-2.28 (m, 2H), 2.05-2.02 (d, J=12.0 Hz, 6H), 1.78 (d, J=6.8 Hz, 2H), 1.64-1.61 (m, 2H), 1.36-1.25 (m, 27H).

### Step F. Procedure for Preparation of 14-[4-[6-[8-(1,3-benzothiazol-2-ylcarbamoyl)-3,4-dihydro-1H-isoquinolin-2-yl]-2-tert-butoxycarbonyl-3-pyridyl]-3,5-dimethyl-pyrazol-1-yl]tetradecanoic acid

To a solution of tert-butyl 6-[8-(1,3-benzothiazol-2-ylcarbamoyl)-3,4-dihydro-1H-isoquinolin-2-yl]-3-[1-(14-methoxy-14-oxo-tetradecyl)-3,5-dimethyl-pyrazol-4-yl]pyridine-2-carboxylate (0.2 g, 243.58 μmol, 1 equiv.) in THF (2 mL) and water (0.5 mL) was added LiOH·H₂O (204.41 mg, 4.87 mmol, 20 equiv.). The mixture was stirred at 25° C. for 1 hour. The reaction mixture was filtered and concentrated under the reduced pressure to give 14-[4-[6-[8-(1,3-benzothiazol-2-ylcarbamoyl)-3,4-dihydro-1H-isoquinolin-2-yl]-2-tert-butoxycarbonyl-3-pyridyl]-3,5-dimethyl-pyrazol-1-yl]tetradecanoic acid (0.2 g, crude) as a brown gum.

MS (ESI) m/z: 807.4 [M+H]⁺.

### Step G. Procedure for Preparation of tert-butyl 6-[8-(1,3-benzothiazol-2-ylcarbamoyl)-3,4-dihydro-1H-isoquinolin-2-yl]-3-[1-[14-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-14-oxo-tetradecyl]-3,5-dimethyl-pyrazol-4-yl]pyridine-2-carboxylate

To a solution of 14-[4-[6-[8-(1,3-benzothiazol-2-ylcarbamoyl)-3,4-dihydro-1H-isoquinolin-2-yl]-2-tert-butoxycarbonyl-3-pyridyl]-3,5-dimethyl-pyrazol-1-yl]tetradecanoic acid (0.2 g, 247.82 μmol, 1 equiv.) and (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (110.18 mg, 247.82 μmol, 1 equiv.) in DMF (2 mL) was added HATU (141.34 mg, 371.72 μmol, 1.5 equiv.) and DIPEA (96.08 mg, 743.45 μmol, 129.49 μL, 3 equiv.). The mixture was stirred at 25° C. for 2 hours to give a brown solution. The reaction mixture was quenched by water (3 mL) and extracted with ethyl acetate 9 mL (3 mL×3). The combined organic layers were washed with brine 9 mL (3 mL×3), dried over sodium sulfate, filtered, and concentrated under reduced pressure to give a residue. The residue was purified by flash silica gel chromatography (eluent of 0˜20% MeOH/DCM) to give tert-butyl 6-[8-(1,3-benzothiazol-2-ylcarbamoyl)-3,4-dihydro-1H-isoquinolin-2-yl]-3-[1-[14-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-14-oxo-tetradecyl]-3,5-dimethyl-pyrazol-4-yl]pyridine-2-carboxylate (70 mg, 52.4 μmol, 21.1% yield, 92.4% purity) as a brown oil.

MS (ESI) m/z: 1234.5 [M+H]⁺.

¹H NMR (400 MHz, CDCl₃) δ=8.69 (s, 1H), 7.88 (d, J=8.4 Hz, 1H), 7.74 (d, J=8.0 Hz, 1H), 7.57 (d, J=7.6 Hz, 1H), 7.43-7.41 (m, 4H), 7.40-7.34 (m, 4H), 6.86 (d, J=8.8 Hz, 1H), 6.34-6.22 (m, 1H), 5.32 (s, 1H), 5.13-4.99 (m, 3H), 4.70 (s, 1H), 4.60-4.52 (m, 2H), 4.22-4.14 (m, 1H), 4.08 (s, 2H), 4.03-3.94 (m, 2H), 3.60 (dd, J=3.2, 10.8 Hz, 1H), 3.08 (t, J=5.2 Hz, 2H), 2.63-2.57 (m, 1H), 2.56-2.53 (m, 3H), 2.19 (s, 2H), 2.07 (s, 3H), 2.04 (s, 3H), 1.80 (dd, J=1.6, 3.2 Hz, 2H), 1.49 (d, J=6.8 Hz, 5H), 1.31-1.23 (m, 27H), 1.07 (s, 9H).

### Step H. Procedure for Preparation of 6-[8-(1,3-benzothiazol-2-ylcarbamoyl)-3,4-dihydro-1H-isoquinolin-2-yl]-3-[1-[14-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-14-oxo-tetradecyl]-3,5-dimethyl-pyrazol-4-yl]pyridine-2-carboxylic acid

A solution of tert-butyl 6-[8-(1,3-benzothiazol-2-ylcarbamoyl)-3,4-dihydro-1H-isoquinolin-2-yl]-3-[1-[14-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-14-oxo-tetradecyl]-3,5-dimethyl-pyrazol-4-yl]pyridine-2-carboxylate (50 mg, 40.53 μmol, 1 equiv.) in TFA (0.5 mL) and DCM (1 mL) was stirred at 25° C. for 3 hours to give a brown solution. The reaction mixture was filtered and concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[1-[14-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-14-oxo-tetradecyl]-3,5-dimethyl-pyrazol-4-yl]pyridine-2-carboxylic acid (18.6 mg, 15.6 μmol, 38.7% yield, 98.9% purity) as a yellow solid.

MS (ESI) m/z: 1178.1 [M+H]⁺.

¹H NMR (400 MHz, CD₃OD) δ=8.87 (s, 1H), 7.92 (d, J=7.6 Hz, 1H), 7.78 (d, J=8.0 Hz, 1H), 7.64-7.58 (m, 1H), 7.49-7.33 (m, 9H), 7.08 (d, J=8.8 Hz, 1H), 5.09 (s, 2H), 5.04-4.97 (m, 1H), 4.64-4.59 (m, 1H), 4.56 (t, J=8.4 Hz, 1H), 4.42 (s, 1H), 4.06-3.97 (m, 4H), 3.87 (d, J=11.2 Hz, 1H), 3.74 (dd, J=4.0, 11.2 Hz, 1H), 3.16-3.05 (m, 2H), 2.50-2.42 (m, 3H), 2.31-2.21 (m, 2H), 2.20-2.14 (m, 1H), 2.05 (d, J=16.4 Hz, 6H), 2.00-1.91 (m, 1H), 1.82-1.74 (m, 2H), 1.62-1.56 (m, 2H), 1.50 (d, J=7.2 Hz, 3H), 1.29 (s, 18H), 1.06-0.99 (m, 9H).

### Example 7. Preparation of Compound 106a

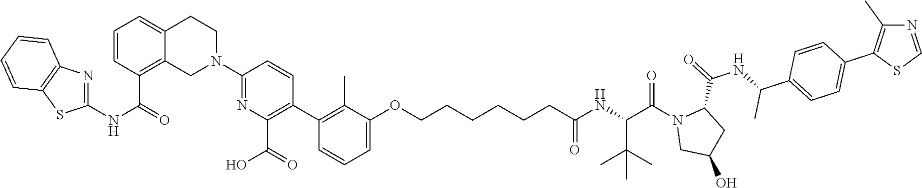 [SMILES: Cc1ncsc1-c1ccc([C@H](C)NC(=O)[C@@H]2C[C@@H](O)CN2C(=O)[C@@H](NC(=O)CCCCCCOc2cccc(-c3ccc(N4CCc5cccc(C(=O)Nc6nc7ccccc7s6)c5C4)nc3C(=O)O)c2C)C(C)(C)C)cc1]

### 6-(8-(benzo[d]thiazol-2-ylcarbamoyl)-3,4-dihydroisoquinolin-2(1H)-yl)-3-(3-((7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptyl)oxy)-2-methylphenyl)picolinic acid

### Step A. Procedure for Preparation of methyl 7-(3-bromo-2-methyl-phenoxy) heptanoate

To a solution of methyl 7-bromoheptanoate (1.79 g, 8.02 mmol, 1.5 equiv.) in MeCN (10 mL) was added K₂CO₃ (2.22 g, 16.04 mmol, 3 equiv.) and 3-bromo-2-methyl-phenol (1 g, 5.35 mmol, 1 equiv.). The mixture was stirred at 60° C. for 2 hours. The reaction mixture was concentrated under reduced pressure to remove MeCN and to give a residue. The residue was diluted with water (20 mL) and extracted with ethyl acetate 30 mL (10 mL×3). The combined organic layers were washed with water 15 mL (5 mL×3) to give a residue. The residue was purified by flash silica gel chromatography (eluent of 0-38% ethyl acetate/petroleum ether) to give methyl 7-(3-bromo-2-methyl-phenoxy) heptanoate (1.8 g, 3.8 mmol, 71.5% yield, 70% purity) as a brown solid.

### Step B. Procedure for Preparation of tert-butyl 6-[8-(1,3-benzothiazol-2-ylcarbamoyl)-3,4-dihydro-1H-isoquinolin-2-yl]-3-[3-(7-methoxy-7-oxo-heptoxy)-2-methyl-phenyl]pyridine-2-carboxylate

A mixture of methyl 7-(3-bromo-2-methyl-phenoxy) heptanoate (350 mg, 701.64 μmol, 66% purity, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (382.03 mg, 561.31 μmol, 90% purity, 1.2 equiv.), Ad₂nBuP Pd G3(cataCXium® A Pd G3) (102.20 mg, 140.33 μmol, 0.2 equiv.), and K₂CO₃ (1.5 M, 701.64 μL, 1.5 equiv.) in 1,4-dioxane (7 mL) was degassed and purged with N₂ three times, and then the mixture was stirred at 80° C. for 3 hours. The reaction mixture was concentrated under reduced pressure to remove 1,4-dioxane and to give a residue. The residue was diluted with water (3 mL) and extracted with ethyl acetate 20 mL (10 mL×2). The combined organic layers were washed with water (10 mL), dried over anhydrous sodium sulfate, filtered, and concentrated under reduced pressure to give a crude product tert-butyl 6-[8-(1,3-benzothiazol-2-ylcarbamoyl)-3,4-dihydro-1H-isoquinolin-2-yl]-3-[3-(7-methoxy-7-oxo-heptoxy)-2-methyl-phenyl]pyridine-2-carboxylate (480 mg, crude) as a white solid, which was used in the next step without further purification.

MS (ESI) m/z: 735.7 [M+H]⁺.

### Step C. Procedure for Preparation of 7-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]heptanoic acid

A mixture of tert-butyl 6-[8-(1,3-benzothiazol-2-ylcarbamoyl)-3,4-dihydro-1H-isoquinolin-2-yl]-3-[3-(7-methoxy-7-oxo-heptoxy)-2-methyl-phenyl]pyridine-2-carboxylate (300 mg, 408.22 μmol, 1 equiv.), LiOH·H₂O (51.39 mg, 1.22 mmol, 6 equiv.) in THF (2.5 mL) and water (0.5 mL) was stirred at 20° C. for 12 hour. The reaction mixture was concentrated under reduced pressure to remove THF to give a residue. The residue was diluted with water (3 mL) and extracted with ethyl acetate 12 mL (4 mL×3). The combined organic layers were washed with water (5 mL) and concentrated under reduced pressure to give a residue. The residue was used in the next step without further purification. The compound 7-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]heptanoic acid (500 mg, crude) was obtained as a yellow solid.

MS (ESI) m/z: 721.4 [M+H]⁺.

### Step D. Procedure for Preparation of tert-butyl 6-(8-(benzo[d]thiazol-2-ylcarbamoyl)-3,4-dihydroisoquinolin-2(1H)-yl)-3-(3-((7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptyl)oxy)-2-methylphenyl)picolinate

A mixture of 7-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]heptanoic acid (100 mg, 106.81 μmol, 77% purity, 1 equiv.), (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (56.99 mg, 128.18 μmol, 1.2 equiv.), HATU (40.61 mg, 106.81 μmol, 1 equiv.), and DIPEA (41.41 mg, 320.44 μmol, 55.82 μL, 3 equiv.) in DMF (2 mL) was degassed and purged with N₂ three times, and then the mixture was stirred at 25° C. for 2 hours under N₂ atmosphere. The mixture was diluted with water (20 mL) and extracted with ethyl acetate 21 mL (7 mL×3). The combined organic layers were washed with water 21 mL (7 mL×3) and concentrated under reduced pressure to give a residue. The residue was used in the next step without further purification. tert-Butyl 6-(8-(benzo[d]thiazol-2-ylcarbamoyl)-3,4-dihydroisoquinolin-2(1H)-yl)-3-(3-((7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptyl)oxy)-2-methylphenyl)picolinate (100 mg, crude) was obtained as a white solid.

MS (ESI) m/z: 1147.5[M+H]⁺.

### Step E. Procedure for Preparation of 6-(8-(benzo[d]thiazol-2-ylcarbamoyl)-3,4-dihydroisoquinolin-2(1H)-yl)-3-(3-((7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptyl)oxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-((7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptyl)oxy)-2-methylphenyl)picolinate (60 mg, 52.29 μmol, 1 equiv.) in acetic acid (2 mL) was stirred at 70° C. for 1 hour under N₂ atmosphere. The reaction mixture was concentrated under reduced pressure to give a residue. The residue was purified by prep-HPLC. 6-(8-(Benzo[d]thiazol-2-ylcarbamoyl)-3,4-dihydroisoquinolin-2(l1)-yl)-3-(3-((7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptyl)oxy)-2-methylphenyl)picolinic acid (24.9 mg, 18.9 μmol, 36.2% yield, 89.7% purity) was obtained as a white solid.

MS (ESI) m/z: 546.5 [M12+H]⁺.

¹H NMR (400 MHz, CD₃OD) δ=8.89-8.87 (m, 1H), 8.53-8.48 (m, 1H), 8.09 (s, 1H), 7.93 (d, J=7.6 Hz, 1H), 7.80 (d, J=7.2 Hz, 1H), 7.63 (d, J=7.6 Hz, 1H), 7.48-7.41 (m, 8H), 7.10 (d, J=8.4 Hz, 2H), 6.87 (d, J=8.4 Hz, 1H), 6.67 (d, J=7.2 Hz, 1H), 5.11 (s, 2H), 4.63 (s, 1H), 4.59-4.56 (m, 1H), 4.45-4.42 (m, 1H), 4.02 (d, J=6.0 Hz, 4H), 3.89 (d, J=11.6 Hz, 1H), 3.75 (d, J=6.8 Hz, 1H), 3.14 (d, J=6.0 Hz, 3H), 2.49 (s, 2H), 2.33-2.29 (m, 2H), 2.21-2.14 (m, 1H), 1.96 (s, 3H), 1.85-1.81 (m, 2H), 1.69-1.64 (m, 2H), 1.54-1.50 (m, 4H), 1.47-1.42 (m, 2H), 1.31 (s, 1H), 1.04 (s, 9H).

### Example 8. Preparation of Compound 107a

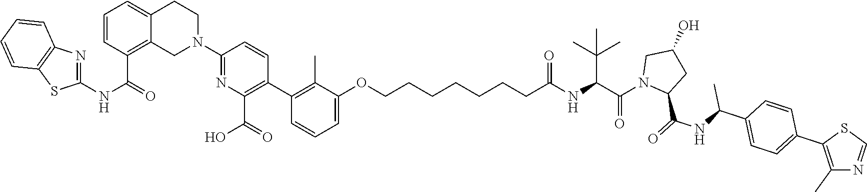 [SMILES: Cc1ncsc1-c1ccc([C@H](C)NC(=O)[C@@H]2C[C@@H](O)CN2C(=O)[C@@H](NC(=O)CCCCCCCOc2cccc(-c3ccc(N4CCc5cccc(C(=O)Nc6nc7ccccc7s6)c5C4)nc3C(=O)O)c2C)C(C)(C)C)cc1]

### 6-[8-(1,3-benzothiazol-2-ylcarbamoyl)-3,4-dihydro-1H-isoquinolin-2-yl]-3-[3-[8-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-8-oxo-octoxy]-2-methyl-phenyl]pyridine-2-carboxylic acid

### Step A. Procedure for Preparation of ethyl 8-(3-bromo-2-methyl-phenoxy)octanoate

A mixture of ethyl 8-bromooctanoate (2 g, 7.96 mmol, 1 equiv.), 3-bromo-2-methyl-phenol (1.79 g, 9.56 mmol, 1.2 equiv.) and K₂CO₃ (3.30 g, 23.89 mmol, 3 equiv.) in MeCN (40 mL) was degassed and purged with N₂ three times, and then the mixture was stirred at 60° C. for 2 hours under N₂ atmosphere. TLC indicated ethyl 8-bromooctanoate was consumed completely and one new spot formed. The reaction mixture was concentrated under reduced pressure to give a residue and then diluted with water 20 mL and extracted with ethyl acetate 90 mL (30 mL×3). The combined organic layers were washed with brine 60 mL (20 mL×3), dried over anhydrous sodium sulfate, filtered and concentrated under reduced pressure to give a residue. The residue was purified by column chromatography (SiO₂, petroleum ether/ethyl acetate=20/1 to 10/1). Ethyl 8-(3-bromo-2-methyl-phenoxy)octanoate (2.1 g, 5.2 mmol, 66.4% yield, 90% purity) was obtained as a colorless oil.

¹H NMR (400 MHz, CD₃OD) δ=7.19-7.15 (m, 1H), 7.07 (t, J=8.0 Hz, 1H), 6.92 (d, J=8.0 Hz, 1H), 4.03 (t, J=6.4 Hz, 2H), 2.39-2.36 (m, 2H), 2.35 (s, 3H), 1.93-1.82 (m, 3H), 1.74-1.64 (m, 3H), 1.62-1.51 (m, 3H), 1.49-1.39 (m, 6H)

### Step B. Procedure for Preparation of tert-butyl 6-[8-(1,3-benzothiazol-2-ylcarbamoyl)-3,4-dihydro-1H-isoquinolin-2-yl]-3-[3-(8-ethoxy-8-oxo-octoxy)-2-methyl-phenyl]pyridine-2-carboxylate

Ethyl 8-(3-bromo-2-methyl-phenoxy)octanoate (300 mg, 839.67 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (503.16 mg, 755.71 μmol, 92% purity, 0.9 equiv.), Ad₂nBuP Pd G₃(cataCXium® APd G₃) (61.15 mg, 83.97 μmol, 0.1 equiv.), and KF (1.5 M, 1.68 mL, 3 equiv.) were taken up into a microwave tube in dioxane (5.5 mL). The sealed tube was heated at 100° C. for 120 minutes under microwave. The reaction mixture was concentrated under reduced pressure to give a residue. The residue was purified by column chromatography (SiO₂, petroleum ether/ethyl acetate=20/1 to 10/1). The compound tert-butyl 6-[8-(1,3-benzothiazol-2-ylcarbamoyl)-3,4-dihydro-1H-isoquinolin-2-yl]-3-[3-(8-ethoxy-8-oxo-octoxy)-2-methyl-phenyl]pyridine-2-carboxylate (490 mg, 578.0 μmol, 68.8% yield, 90% purity) was obtained as a yellow solid.

MS (ESI) m/z: 764.3 [M+H]⁺

¹H NMR (400 MHz, CD₃OD) δ=7.95 (d, J=7.6 Hz, 1H), 7.81 (d, J=8.0 Hz, 1H), 7.62 (d, J=7.6 Hz, 1H), 7.50-7.37 (m, 5H), 7.17-7.10 (m, 1H), 7.00 (d, J=8.8 Hz, 1H), 6.92 (d, J=8.0 Hz, 1H), 6.65 (d, J=7.6 Hz, 1H), 5.07 (s, 2H), 4.84 (s, 2H), 4.07-4.01 (m, 4H), 3.13 (t, J=6.0 Hz, 2H), 2.34 (t, J=7.6 Hz, 2H), 1.97 (s, 3H), 1.87-1.80 (m, 2H), 1.69-1.62 (m, 2H), 1.59-1.52 (m, 3H), 1.45-1.38 (m, 4H), 1.21 (d, J=5.6 Hz, 2H), 1.12 (s, 9H)

### Step C. Procedure for Preparation of 8-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]octanoic acid

A mixture of tert-butyl 6-[8-(1,3-benzothiazol-2-ylcarbamoyl)-3,4-dihydro-1H-isoquinolin-2-yl]-3-[3-(8-ethoxy-8-oxo-octoxy)-2-methyl-phenyl]pyridine-2-carboxylate (490 mg, 642.24 μmol, 1 equiv.) and LiOH·H₂O (80.85 mg, 1.93 mmol, 3 equiv.) in THF (4 mL) and water (1 mL) was degassed and purged with N₂ three times, and then the mixture was stirred at 25° C. for 12 hours under N₂ atmosphere. The reaction mixture was then diluted with water (50 mL) and extracted with ethyl acetate 90 mL (30 mL×3). The combined organic layers were washed with brine 60 mL (20 mL×3), dried over anhydrous sodium sulfate, filtered, and concentrated under reduced pressure to give 8-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]octanoic acid (400 mg, crude) as an off-white solid.

MS (ESI) m/z: 735.8 [M+H]⁺

### Step D. Procedure for Preparation of tert-butyl 6-[8-(1,3-benzothiazol-2-ylcarbamoyl)-3,4-dihydro-1H-isoquinolin-2-yl]-3-[3-[8-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-8-oxo-octoxy]-2-methyl-phenyl]pyridine-2-carboxylate

A mixture of 8-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]octanoic acid (200 mg, 272.15 μmol, 1 equiv.), (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (133.09 mg, 299.37 μmol, 1.1 equiv.), and DIPEA (105.52 mg, 816.45 μmol, 142.21 μL, 3 equiv.) in DMF (2.3 mL) was degassed and purged with N₂ three times, the mixture was stirred at 25° C. for 5 minutes. After 5 minutes, HATU (124.17 mg, 326.58 μmol, 1.2 equiv.) was added, and the mixture was stirred at 25° C. for 2 hours under N₂ atmosphere. The reaction mixture was then diluted with water (50 mL) and extracted with ethyl acetate 90 mL (30 mL×3). The combined organic layers were washed with brine 60 mL (20 mL×3), dried over anhydrous sodium sulfate, filtered, and concentrated under reduced pressure to give tert-butyl 6-[8-(1,3-benzothiazol-2-ylcarbamoyl)-3,4-dihydro-1H-isoquinolin-2-yl]-3-[3-[8-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-8-oxo-octoxy]-2-methyl-phenyl]pyridine-2-carboxylate (280 mg, crude) as an off-white solid.

MS (ESI) m/z: 581.5 [M12+H]⁺

¹H NMR (400 MHz, CD₃OD) δ=7.90 (d, J=8.4 Hz, 1H), 7.76 (d, J=8.0 Hz, 1H), 7.60 (d, J=8.0 Hz, 1H), 7.46-7.30 (m, 10H), 7.11 (t, J=8.0 Hz, 1H), 6.97 (d, J=8.8 Hz, 1H), 6.90 (d, J=8.4 Hz, 1H), 6.63 (d, J=7.6 Hz, 1H), 5.06-5.02 (m, 2H), 4.65-4.61 (m, 1H), 4.58 (t, J=8.4 Hz, 1H), 4.44 (s, 1H), 4.01 (d, J=5.6 Hz, 4H), 3.89 (d, J=10.8 Hz, 1H), 3.75 (dd, J=10.8, 4.0 Hz, 1H), 3.12-3.05 (m, 3H), 2.82-2.73 (m, 1H), 2.49 (s, 3H), 2.35-2.20 (m, 3H), 1.97-1.94 (m, 3H), 1.84-1.79 (m, 2H), 1.68-1.61 (m, 2H), 1.53-1.50 (m, 4H), 1.43-1.36 (m, 4H), 1.17 (d, J=6.4 Hz, 6H), 1.12-1.08 (m, 8H), 1.07-1.02 (m, 9H)

### Step E. Procedure for Preparation of 6-[8-(1,3-benzothiazol-2-ylcarbamoyl)-3,4-dihydro-1H-isoquinolin-2-yl]-3-[3-[8-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-8-oxo-octoxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[8-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-8-oxo-octoxy]-2-methyl-phenyl]pyridine-2-carboxylate (110 mg, 94.71 μmol, 1 equiv.) in DCM (0.55 mL) and TFA (0.55 mL) was degassed and purged with N₂ three times, and then the mixture was stirred at 25° C. for 24 hours under N₂ atmosphere. The reaction mixture was concentrated under reduced pressure to give a residue. The residue was purified by prep-HPLC. 6-[8-(1,3-Benzothiazol-2-ylcarbamoyl)-3,4-dihydro-1H-isoquinolin-2-yl]-3-[3-[8-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-8-oxo-octoxy]-2-methyl-phenyl]pyridine-2-carboxylic acid (27.3 mg, 22.0 μmol, 23.2% yield, 89% purity) was obtained as a yellow solid.

MS (ESI) m/z: 1105.4 [M+H]⁺

¹H NMR (400 MHz, DMSO-d₆) δ=12.84-12.46 (m, 1H), 8.98 (s, 1H), 8.35 (d, J=8.0 Hz, 1H), 8.03 (d, J=8.0 Hz, 1H), 7.82-7.73 (m, 2H), 7.62 (d, J=6.8 Hz, 1H), 7.50-7.31 (m, 10H), 7.13-7.05 (m, 1H), 6.99-6.92 (m, 1H), 6.87 (d, J=8.4 Hz, 1H), 6.67-6.58 (m, 1H), 5.01-4.95 (m, 2H), 4.95-4.88 (m, 1H), 4.54-4.47 (m, 1H), 4.45-4.38 (m, 1H), 4.27 (d, J=1.6 Hz, 1H), 3.98-3.89 (m, 4H), 3.59 (s, 2H), 3.05-3.00 (m, 2H), 2.45 (s, 4H), 1.89 (s, 4H), 1.82-1.69 (m, 4H), 1.50-1.41 (m, 4H), 1.39-1.34 (m, 4H), 1.32-1.22 (m, 4H), 0.92 (s, 9H)

### Example 9. Preparation of Compound 108a

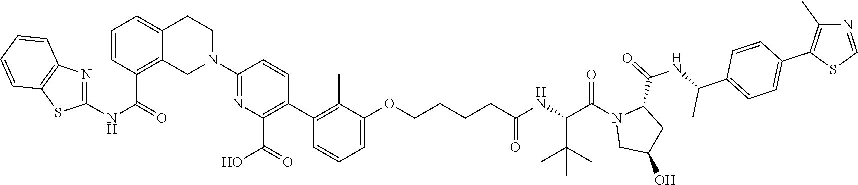 [SMILES: Cc1ncsc1-c1ccc([C@H](C)NC(=O)[C@@H]2C[C@@H](O)CN2C(=O)[C@@H](NC(=O)CCCCOc2cccc(-c3ccc(N4CCc5cccc(C(=O)Nc6nc7ccccc7s6)c5C4)nc3C(=O)O)c2C)C(C)(C)C)cc1]

### 6-[8-(1,3-benzothiazol-2-ylcarbamoyl)-3,4-dihydro-1H-isoquinolin-2-yl]-3-[3-[5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-5-oxo-pentoxy]-2-methyl-phenyl]pyridine-2-carboxylic acid

### Step A. Procedure for Preparation of methyl 5-(3-bromo-2-methyl-phenoxy)pentanoate

A mixture of methyl 5-bromopentanoate (2 g, 10.25 mmol, 1.47 mL, 1 equiv.), 3-bromo-2-methyl-phenol (2.11 g, 11.28 mmol, 1.1 equiv.) and K₂CO₃ (4.25 g, 30.76 mmol, 3 equiv.) in ACN (40 mL) was degassed and purged with N₂ three times, and then the mixture was stirred at 60° C. for 12 hours under N₂ atmosphere. The reaction mixture was filtered and concentrated under reduced pressure to give a residue, and then the reaction mixture was diluted with water (20 mL) and extracted with ethyl acetate 100 mL (20 mL×5). The combined organic layers were washed with brine 20 mL (10 mL×2), dried over sodium sulfate, filtered, and concentrated under reduced pressure to give a residue. The residue was purified by column chromatography (SiO₂, petroleum ether/ethyl acetate=1/0 to 97/3) to give compound methyl 5-(3-bromo-2-methyl-phenoxy)pentanoate (2.9 g, 8.6 mmol, 84.5% yield, 90% purity) as a colorless oil.

¹H NMR (400 MHz, CDCl₃) δ=7.14 (d, J=8.0 Hz, 1H), 6.99 (t, J=8.0 Hz, 1H), 6.75 (d, J=8.4 Hz, 1H), 3.97 (t, J=5.2 Hz, 2H), 3.69 (s, 3H), 2.44-2.40 (m, 2H), 2.32 (s, 3H), 1.91-1.79 (m, 4H).

### Step B. Procedure for Preparation of tert-butyl 6-[8-(1,3-benzothiazol-2-ylcarbamoyl)-3,4-dihydro-1H-isoquinolin-2-yl]-3-[3-(5-methoxy-5-oxo-pentoxy)-2-methyl-phenyl]pyridine-2-carboxylate

To a mixture of tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (350 mg, 571.39 μmol, 1 equiv.) and methyl 5-(3-bromo-2-methyl-phenoxy)pentanoate (229.45 mg, 685.66 μmol, 90% purity, 1.2 equiv.) in dioxane (3 mL) was added KF (1.5 M, 1.14 mL, 3 equiv.) and Ad₂nBuP Pd G₃(cataCXium® A Pd G₃) (41.61 mg, 57.14 μmol, 0.1 equiv.). After the addition, the mixture was degassed and purged with N₂ three times, and then the mixture was stirred at 100° C. for 2 hours under N₂ atmosphere. The reaction mixture was diluted with water (6 mL) and extracted with ethyl acetate 20 mL (5 mL×4). The combined organic layers were washed with brine 10 mL (5 mL×2), dried over sodium sulfate, filtered and concentrated under reduced pressure to give a residue. The residue was purified by column chromatography (SiO₂, petroleum ether/ethyl acetate=1/0 to 76/24) to give compound tert-butyl 6-[8-(1,3-benzothiazol-2-ylcarbamoyl)-3,4-dihydro-1H-isoquinolin-2-yl]-3-[3-(5-methoxy-5-oxo-pentoxy)-2-methyl-phenyl]pyridine-2-carboxylate (250 mg, 325.2 μmol, 56.9% yield, 91.9% purity) as a yellow oil.

MS (ESI) m/z: 707.8 [M+H]⁺.

¹H NMR (400 MHz, CDCl₃) δ=7.87 (d, J=7.6 Hz, 1H), 7.71 (dd, J=7.6, 8.0 Hz, 2H), 7.47 (t, J=8.0 Hz, 1H), 7.41-7.34 (m, 4H), 7.10 (t, J=8.0 Hz, 1H), 6.90 (d, J=8.4 Hz, 1H), 6.81 (d, J=8.0 Hz, 1H), 6.70 (d, J=7.6 Hz, 1H), 5.15-5.00 (m, 2H), 4.11-4.09 (m, 2H), 4.03-3.99 (m, 2H), 3.68 (s, 3H), 3.08 (t, J=5.6 Hz, 2H), 2.44-2.40 (m, 2H), 2.00 (s, 3H), 1.88-1.85 (m, 4H), 1.16 (s, 9H)

### Step C. Procedure for Preparation of 5-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]pentanoic acid

To a solution of tert-butyl 6-[8-(1,3-benzothiazol-2-ylcarbamoyl)-3,4-dihydro-1H-isoquinolin-2-yl]-3-[3-(5-methoxy-5-oxo-pentoxy)-2-methyl-phenyl]pyridine-2-carboxylate (250 mg, 353.68 μmol, 1 equiv.) in THF (2.5 mL) was added LiOH·H₂O (25.41 mg, 1.06 mmol, 3 equiv.) and water (0.6 mL) at 25° C. The mixture was stirred at 25° C. for 12 hours. The reaction mixture was diluted with water 5 mL and extracted with ethyl acetate 15 mL (5 mL×3). The combined organic layers were washed with brine 15 mL (5 mL×3), dried over sodium sulfate, filtered, and concentrated under reduced pressure to give 5-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]pentanoic acid (240 mg, crude) as a yellow oil.

MS (ESI) m/z: 693.4 [M+H]⁺.

### Step D. Procedure for Preparation of tert-butyl 6-[8-(1,3-benzothiazol-2-ylcarbamoyl)-3,4-dihydro-1H-isoquinolin-2-yl]-3-[3-[5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-5-oxo-pentoxy]-2-methyl-phenyl]pyridine-2-carboxylate

To a solution of (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (154.01 mg, 346.41 μmol, 1.2 equiv.) in DMF (2 mL) was added HATU (131.72 mg, 346.41 μmol, 1.2 equiv.), DIPEA (111.92 mg, 866.02 μmol, 150.84 μL, 3 equiv.), and 5-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]pentanoic acid (200 mg, 288.67 μmol, 1 equiv.). The mixture was stirred at 25° C. for 3 hours. The reaction mixture was quenched by addition water (4 mL). The reaction mixture was filtered and the filter cake was washed with water (10 mL). After washing, the filter cake was diluted in DCM (20 mL). The combined organic layers were dried over sodium sulfate, filtered, and concentrated under reduced pressure to give compound tert-butyl 6-[8-(1,3-benzothiazol-2-ylcarbamoyl)-3,4-dihydro-1H-isoquinolin-2-yl]-3-[3-[5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-5-oxo-pentoxy]-2-methyl-phenyl]pyridine-2-carboxylate (350 mg, crude) as a yellow solid.

MS (ESI) m/z: 1120.4 [M+H]⁺.

### Step E. Procedure for Preparation of 6-[8-(1,3-benzothiazol-2-ylcarbamoyl)-3,4-dihydro-1H-isoquinolin-2-yl]-3-[3-[5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-5-oxo-pentoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-5-oxo-pentoxy]-2-methyl-phenyl]pyridine-2-carboxylate (350 mg, 312.67 μmol, 1 equiv.) in DCM (2 mL) was added TFA (3.21 g, 28.14 mmol, 2.08 mL, 90 equiv.). The mixture was stirred at 25° C. for 12 hours. The reaction mixture was concentrated under reduced pressure to give a residue. The crude product was purified by prep-HPLC to give compound 6-[8-(1,3-benzothiazol-2-ylcarbamoyl)-3,4-dihydro-1H-isoquinolin-2-yl]-3-[3-[5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-5-oxo-pentoxy]-2-methyl-phenyl]pyridine-2-carboxylic acid (119.7 mg, 110.3 μmol, 35.2% yield, 97.9% purity) as a yellow solid.

MS (ESI) m/z: 1063.4 [M+H]⁺

¹H NMR (400 MHz, DMSO-d₆) δ=12.88-12.82 (m, 1H), 8.98 (s, 1H), 8.37 (d, J=7.6 Hz, 1H), 8.03 (d, J=8.0 Hz, 1H), 7.85 (d, J=9.6 Hz, 1H), 7.79 (d, J=8.0 Hz, 1H), 7.62 (d, J=7.2 Hz, 1H), 7.50-7.34 (m, 10H), 7.12-7.07 (m, 1H), 6.97 (d, J=8.8 Hz, 1H), 6.88 (d, J=8.0 Hz, 1H), 6.63 (d, J=7.2 Hz, 1H), 5.09 (d, J=3.2 Hz, 1H), 4.98 (s, 2H), 4.92 (t, J=7.2 Hz, 1H), 4.53 (d, J=9.6 Hz, 1H), 4.42 (t, J=8.0 Hz, 1H), 4.27 (s, 1H), 3.97-3.93 (m, 2H), 3.92 (t, J=6.0 Hz, 2H), 3.64-3.58 (m, 2H), 3.32 (s, 3H), 3.03 (t, J=5.6 Hz, 2H), 2.45 (s, 3H), 2.36-2.32 (m, 1H), 2.20 (td, J=6.8, 7.2 Hz, 1H), 2.04-1.97 (m, 1H), 1.89 (s, 3H), 1.71 (d, J=6.8 Hz, 3H), 1.37 (d, J=6.8 Hz, 2H), 0.93 (s, 9H)

### Example 10. Preparation of Compound 110a

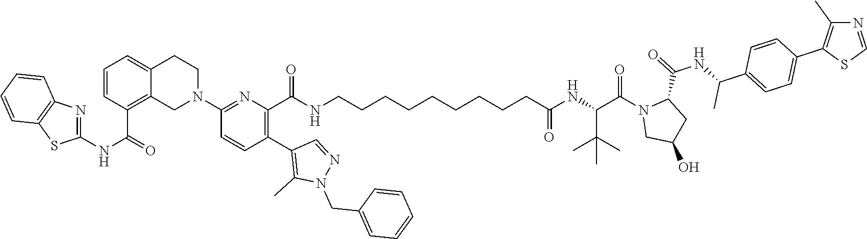 [SMILES: Cc1ncsc1-c1ccc([C@H](C)NC(=O)[C@@H]2C[C@@H](O)CN2C(=O)[C@@H](NC(=O)CCCCCCCCCNC(=O)c2nc(N3CCc4cccc(C(=O)Nc5nc6ccccc6s5)c4C3)ccc2-c2cnn(Cc3ccccc3)c2C)C(C)(C)C)cc1]

### N-(benzo[d]thiazol-2-yl)-2-(5-(1-benzyl-5-methyl-1H-pyrazol-4-yl)-6-((10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecyl)carbamoyl)pyridin-2-yl)-1,2,3,4-tetrahydroisoquinoline-8-carboxamide

### Step A. Procedure for Preparation of methyl 10-(6-(8-(benzo[d]thiazol-2-ylcarbamoyl)-3,4-dihydroisoquinolin-2(1H)-yl)-3-(1-benzyl-5-methyl-1H-pyrazol-4-yl)picolinamido)decanoate

To a solution of 6-(8-(benzo[d]thiazol-2-ylcarbamoyl)-3,4-dihydroisoquinolin-2(1H)-yl)-3-(1-benzyl-5-methyl-1H-pyrazol-4-yl)picolinic acid (100.00 mg, 166.48 μmol, 1.0 equiv.) in DMF (2 mL) was added DIEA (64.55 mg, 499.43 μmol, 86.99 μL, 3.0 equiv.) and HATU (75.96 mg, 199.77 μmol, 1.2 equiv.). The mixture was stirred at 25° C. for 0.5 hours. To the mixture was added methyl 10-aminodecanoate (47.50 mg, 199.77 μmol, 1.2 equiv., HCl). The mixture was stirred at 25° C. for 0.5 hours. The reaction mixture was poured into water (10 mL), then the mixture was filtered to give methyl 10-(6-(8-(benzo[d]thiazol-2-ylcarbamoyl)-3,4-dihydroisoquinolin-2(1H)-yl)-3-(1-benzyl-5-methyl-1H-pyrazol-4-yl)picolinamido)decanoate (150.00 mg, crude) as an off-white solid, which was carried into the next step without further purification.

MS (ESI) m/z: 784.7 [M+H]⁺.

### Step B. Procedure for Preparation of 10-(6-(8-(benzo[d]thiazol-2-ylcarbamoyl)-3,4-dihydroisoquinolin-2(1H)-yl)-3-(1-benzyl-5-methyl-1H-pyrazol-4-yl)picolinamido)decanoic acid

To a solution of methyl 10-(6-(8-(benzo[d]thiazol-2-ylcarbamoyl)-3,4-dihydroisoquinolin-2(1H)-yl)-3-(1-benzyl-5-methyl-1H-pyrazol-4-yl)picolinamido)decanoate (130.00 mg, 165.82 μmol, 1.0 equiv.) in THF (3 mL) was added LiOH·H₂O (0.5 M, 1.30 mL, 3.92 equiv.). The mixture was stirred at 25° C. for 1 hour. The reaction was acidified with aqueous HCl (1.0 M) to pH 3, and then the mixture was filtered to give 10-(6-(8-(benzo[d]thiazol-2-ylcarbamoyl)-3,4-dihydroisoquinolin-2(1H)-yl)-3-(1-benzyl-5-methyl-1H-pyrazol-4-yl)picolinamido)decanoic acid (120.00 mg, crude) as a white solid, which was used in the next step without further purification.

MS (ESI) m/z: 770.7 [M+H]⁺.

### Step C. Procedure for Preparation of N-(benzo[d]thiazol-2-yl)-2-(5-(1-benzyl-5-methyl-1H-pyrazol-4-yl)-6-((10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecyl)carbamoyl)pyridin-2-yl)-1,2,3,4-tetrahydroisoquinoline-8-carboxamide

To a solution of 10-(6-(8-(benzo[d]thiazol-2-ylcarbamoyl)-3,4-dihydroisoquinolin-2(1H)-yl)-3-(1-benzyl-5-methyl-1H-pyrazol-4-yl)picolinamido)decanoic acid (100.00 mg, 129.88 μmol, 1.0 equiv.) in DMF (1.5 mL) was added DIEA (50.36 mg, 389.63 μmol, 67.87 μL, 3.0 equiv.) and HATU (59.26 mg, 155.85 μmol, 1.2 equiv.). The mixture was stirred at 25° C. for 0.5 hours. To the mixture was added (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (69.29 mg, 155.85 μmol, 1.2 equiv.). The mixture was stirred at 25° C. for 0.5 hours. The reaction mixture was concentrated under reduced pressure to give a residue. The residue was purified by prep-HPLC to give N-(benzo[d]thiazol-2-yl)-2-(5-(1-benzyl-5-methyl-iH-pyrazol-4-yl)-6-((10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecyl)carbamoyl)pyridin-2-yl)-1,2,3,4-tetrahydroisoquinoline-8-carboxamide (36.0 mg, 28.0 μmol, 21.5% yield, 92.9% purity) as a green solid.

MS (ESI) m/z: 1219.1 [M+Na]⁺.

¹H NMR (400 MHz, DMSO-d₆) δ=12.86 (s, 1H), 8.98 (s, 1H), 8.36 (d, J=8.0 Hz, 1H), 8.10 (t, J=6.0 Hz, 1H), 8.01 (d, J=8.0 Hz, 1H), 7.81-7.73 (m, 2H), 7.60 (d, J=7.2 Hz, 1H), 7.49-7.43 (m, 3H), 7.43-7.40 (m, 2H), 7.36 (dd, J=6.8, 14.0 Hz, 5H), 7.32-7.29 (m, 2H), 7.26 (d, J=7.2 Hz, 1H), 7.11 (d, J=7.2 Hz, 2H), 6.97 (d, J=8.8 Hz, 1H), 5.29 (s, 2H), 5.01 (s, 2H), 4.95-4.88 (m, 1H), 4.52 (d, J=9.2 Hz, 1H), 4.44-4.40 (m, 1H), 4.27 (s, 1H), 3.89 (t, J=5.6 Hz, 2H), 3.60 (s, 2H), 3.04-2.98 (m, 4H), 2.45 (s, 2H), 2.27-2.20 (m, 1H), 2.12-2.06 (m, 1H), 2.02 (s, 3H), 1.95-1.87 (m, 1H), 1.82-1.75 (m, 1H), 1.73-1.65 (m, 1H), 1.47-1.41 (m, 2H), 1.37 (d, J=6.8 Hz, 3H), 1.29-1.22 (m, 3H), 1.18-1.05 (m, 10H), 0.93 (s, 9H).

### Example 11. Preparation of Compound 111a

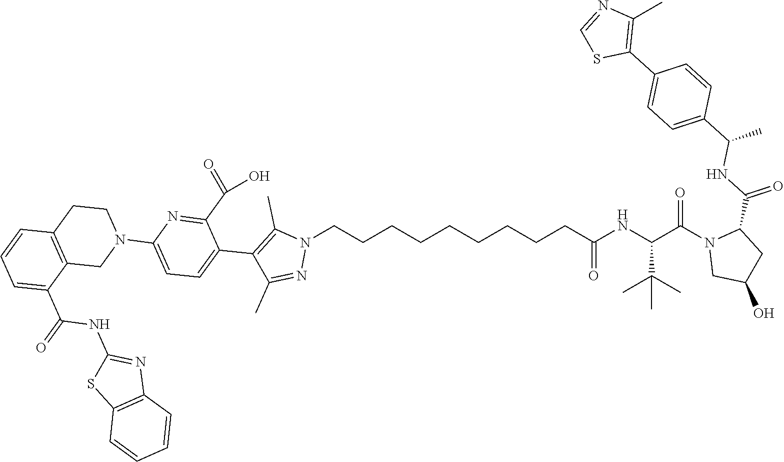 [SMILES: Cc1ncsc1-c1ccc([C@H](C)NC(=O)[C@@H]2C[C@@H](O)CN2C(=O)[C@@H](NC(=O)CCCCCCCCCn2nc(C)c(-c3ccc(N4CCc5cccc(C(=O)Nc6nc7ccccc7s6)c5C4)nc3C(=O)O)c2C)C(C)(C)C)cc1]

### 6-[8-(1,3-benzothiazol-2-ylcarbamoyl)-3,4-dihydro-1H-isoquinolin-2-yl]-3-[1-[10-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-10-oxo-decyl]-3,5-dimethyl-pyrazol-4-yl]pyridine-2-carboxylic acid

### Step A. Procedure for Preparation of methyl 10-bromodecanoate

To a solution of 10-bromodecanoic acid (5 g, 19.91 mmol, 1 equiv.) in MeOH (30 mL) was added SOCl₂ (2.84 g, 23.89 mmol, 1.73 mL, 1.2 equiv.) at 0° C. The mixture was stirred at 65° C. for 12 hours. The reaction mixture was concentrated under reduced pressure to give a residue. The crude product was carried into the next reaction. Methyl 10-bromodecanoate (5.5 g, crude) was obtained as a brown liquid.

¹HNMR (400 MHz, CD₃OD) δ=3.63 (s, 3H), 3.54-3.29 (m, 2H), 2.30 (d, J=6.8 Hz, 2H), 1.82-1.72 (m, 2H), 1.58 (s, 2H), 1.42 (s, 2H), 1.31 (s, 8H).

### Step B. Procedure for Preparation of methyl 10-(4-bromo-3,5-dimethyl-pyrazol-1-yl)decanoate

A mixture of methyl 10-bromodecanoate (2 g, 7.54 mmol, 1 equiv.), 4-bromo-3,5-dimethyl-1H-pyrazole (1.32 g, 7.54 mmol, 1 equiv.), K₂CO₃ (3.13 g, 22.63 mmol, 3 equiv.), and KI (62.60 mg, 377.09 μmol, 0.05 equiv.) in DMF (20 mL) was degassed and purged with N₂ three times, and then the mixture was stirred at 90° C. for 9 hours under N₂ atmosphere. The residue was diluted with water (20 mL) and extracted with ethyl acetate 90 mL (30 mL×3). The combined organic layers were washed with brine 45 mL (15 mL×3), dried over anhydrous sodium sulfate, filtered, and concentrated under reduced pressure to give a residue. The residue was purified by column chromatography (SiO₂, petroleum ether/ethyl acetate=30/1 to 20/1). Methyl 10-(4-bromo-3,5-dimethyl-pyrazol-1-yl)decanoate (1.78 g, 4.9 mmol, 65.6% yield) was obtained as a yellow oil.

MS (ESI) m/z: 360.7 [M+H]⁺.

¹H NMR (400 MHz, CDCl₃) δ=3.90-3.86 (m, 2H), 3.57 (d, J=1.2 Hz, 3H), 2.23-2.19 (m, 2H), 2.14-2.11 (m, 6H), 1.68 (J=6.4 Hz, 2H), 1.52 (J=6.4 Hz, 2H), 1.21 (s, 9H).

### Step C. Procedure for Preparation of tert-butyl 6-[8-(1,3-benzothiazol-2-ylcarbamoyl)-3,4-dihydro-1H-isoquinolin-2-yl]-3-[1-(10-methoxy-10-oxo-decyl)-3,5-dimethyl-pyrazol-4-yl]pyridine-2-carboxylate

A mixture of methyl 10-(4-bromo-3,5-dimethyl-pyrazol-1-yl)decanoate (651.09 mg, 1.81 mmol, 1.5 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (740 mg, 1.21 mmol, 1 equiv.), and K₃PO₄ (1.5 M, 3.06 mL, 3.8 equiv.) in 1,4-dioxane (7 mL) was degassed and purged with N₂ three times, then [2-(2-aminophenyl)phenyl]palladium(1+);bis(1-adamantyl)-butyl-phosphane;methanesulfonate (219.95 mg, 302.02 μmol, 0.25 equiv.) in DCE (1.4 mL) was added. The mixture was stirred at 100° C. for 9 hours under N₂ atmosphere. The reaction mixture was filtered and concentrated under reduced pressure to give a residue. The residue was purified by flash silica gel chromatography (eluent of 0˜45% ethyl acetate/petroleum ether). tert-Butyl 6-[8-(1,3-benzothiazol-2-ylcarbamoyl)-3,4-dihydro-1H-isoquinolin-2-yl]-3-[1-(10-methoxy-10-oxo-decyl)-3,5-dimethyl-pyrazol-4-yl]pyridine-2-carboxylate (478 mg, 624.8 μmol, 51.7% yield) was obtained as a yellow oil.

MS (ESI) m/z: 765.8 [M+H]⁺.

### Step D. Procedure for Preparation of 10-[4-[6-[8-(1,3-benzothiazol-2-ylcarbamoyl)-3,4-dihydro-1H-isoquinolin-2-yl]-2-tert-butoxycarbonyl-3-pyridyl]-3,5-dimethyl-pyrazol-1-yl]decanoic acid

A mixture of tert-butyl 6-[8-(1,3-benzothiazol-2-ylcarbamoyl)-3,4-dihydro-1H-isoquinolin-2-yl]-3-[1-(10-methoxy-10-oxo-decyl)-3,5-dimethyl-pyrazol-4-yl]pyridine-2-carboxylate (200 mg, 261.45 μmol, 1 equiv.) and LiOH·H₂O (18.78 mg, 784.34 μmol, 3 equiv.) in THF (2 mL), MeOH (0.2 mL), and water (0.2 mL) was degassed and purged with N₂ three times, and then the mixture was stirred at 25° C. for 9 hours under N₂ atmosphere. The reaction mixture was washed with toluene 3 mL (1 mL×3), then filtered and concentrated under reduced pressure to give a residue. The residue was purified by prep-TLC (SiO₂, petroleum ether: ethyl acetate=1:2). 10-[4-[6-[8-(1,3-Benzothiazol-2-ylcarbamoyl)-3,4-dihydro-1H-isoquinolin-2-yl]-2-tert-butoxycarbonyl-3-pyridyl]-3,5-dimethyl-pyrazol-1-yl]decanoic acid (86 mg, 114.5 μmol, 43.8% yield) was obtained as a yellow oil.

MS (ESI) m/z: 751.7 [M+H]⁺.

### Step E. Procedure for Preparation of tert-butyl 6-[8-(1,3-benzothiazol-2-ylcarbamoyl)-3,4-dihydro-1H-isoquinolin-2-yl]-3-[1-[10-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-10-oxo-decyl]-3,5-dimethyl-pyrazol-4-yl]pyridine-2-carboxylate

A mixture of 10-[4-[6-[8-(1,3-benzothiazol-2-ylcarbamoyl)-3,4-dihydro-1H-isoquinolin-2-yl]-2-tert-butoxycarbonyl-3-pyridyl]-3,5-dimethyl-pyrazol-1-yl]decanoic acid (86 mg, 114.52 μmol, 1 equiv.), HATU (65.32 mg, 171.78 μmol, 1.5 equiv.), and Et₃N (34.77 mg, 343.57 μmol, 47.82 μL, 3 equiv.) in DMF (0.9 mL) was degassed and purged with N₂ three times and the mixture was stirred at 25° C. for 0.5 hours. (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (50.92 mg, 114.52 μmol, 1 equiv.) was then added, and the mixture was stirred at 25° C. for 2 hours under N₂ atmosphere. The reaction mixture was concentrated under reduced pressure to give a residue. The residue was purified by prep-HPLC. tert-Butyl 6-[8-(1,3-benzothiazol-2-ylcarbamoyl)-3,4-dihydro-1H-isoquinolin-2-yl]-3-[1-[10-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-10-oxo-decyl]-3,5-dimethyl-pyrazol-4-yl]pyridine-2-carboxylate (100 mg, 84.9 μmol, 74.1% yield) was obtained as a white solid.

MS (ESI) m/z: 1177.7 [M+H]⁺.

### Step F. Procedure for Preparation of 6-[8-(1,3-benzothiazol-2-ylcarbamoyl)-3,4-dihydro-1H-isoquinolin-2-yl]-3-[1-[10-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-10-oxo-decyl]-3,5-dimethyl-pyrazol-4-yl]pyridine-2-carboxylic acid

A mixture of tert-butyl 6-[8-(1,3-benzothiazol-2-ylcarbamoyl)-3,4-dihydro-1H-isoquinolin-2-yl]-3-[1-[10-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-10-oxo-decyl]-3,5-dimethyl-pyrazol-4-yl]pyridine-2-carboxylate (90 mg, 76.43 μmol, 1 equiv.) and TFA (43.57 mg, 382.16 μmol, 28.30 μL, 5 equiv.) in CH₂Cl₂ (0.9 mL) was degassed and purged with N₂ three times, and then the mixture was stirred at 25° C. for 2 hours under N₂ atmosphere. The reaction mixture was concentrated under reduced pressure to give a residue. The residue was purified by prep-HPLC. 6-[8-(1,3-Benzothiazol-2-ylcarbamoyl)-3,4-dihydro-1H-isoquinolin-2-yl]-3-[1-[10-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-10-oxo-decyl]-3,5-dimethyl-pyrazol-4-yl]pyridine-2-carboxylic acid (18.2 mg, 16.3 μmol, 21.3% yield) was obtained as a white solid.

MS (ESI) m/z: 1121.2 [M+H]⁺.

¹H NMR (400 MHz, CDCl₃) δ=8.67 (s, 1H), 7.87 (d, J=8.0 Hz, 1H), 7.71-7.65 (m, 1H), 7.59-7.56 (m, 1H), 7.50-7.33 (m, 9H), 7.05-7.02 (m, 1H), 6.66-6.22 (m, 1H), 5.16 (s, 2H), 5.09-5.07 (m, 1H), 4.74-4.70 (m, 1H), 4.58 (d, J=8.8 Hz, 1H), 4.52 (s, 1H), 3.98 (d, J=8.8 Hz, 1H), 3.95-3.92 (m, 4H), 3.59-3.58 (m, 1H), 3.14-3.11 (m, 2H), 2.51 (s, 4H), 2.20-2.19 (m, 2H), 2.05 (d, J=6.0 Hz, 2H), 2.03 (s, 8H), 1.82-1.78 (m, 4H), 1.47 (d, J=6.0 Hz, 3H), 1.29-1.26 (m, 11H), 1.08 (s, 9H).

### Example 12. Preparation of Compound 112a

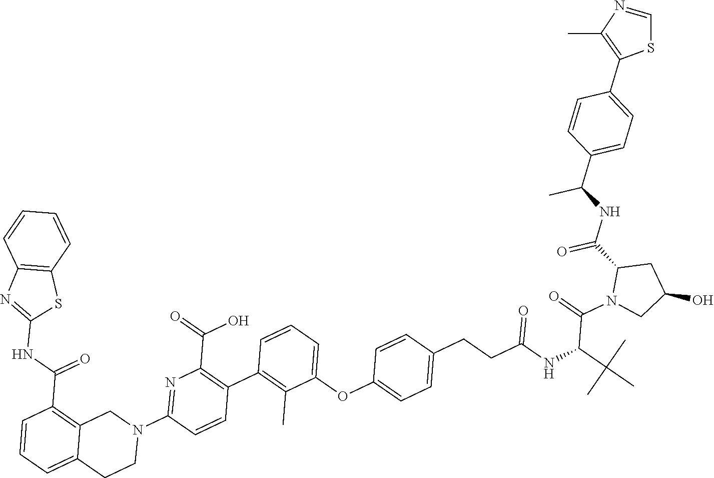 [SMILES: Cc1ncsc1-c1ccc([C@H](C)NC(=O)[C@@H]2C[C@@H](O)CN2C(=O)[C@@H](NC(=O)CCc2ccc(Oc3cccc(-c4ccc(N5CCc6cccc(C(=O)Nc7nc8ccccc8s7)c6C5)nc4C(=O)O)c3C)cc2)C(C)(C)C)cc1]

### 6-[8-(1,3-benzothiazol-2-ylcarbamoyl)-3,4-dihydro-1H-isoquinolin-2-yl]-3-[3-[4-[3-[[(1S)-1-[(2S, 4R)-4-hydroxy-2-[[(1S)-1-[4-(4-methylthiazol-5-yl) phenyl]ethyl]carbamoyl]pyrrolidine-1-carbonyl]-2,2-dimethyl-propyl]amino]-3-oxo-propyl]phenoxy]-2-methyl-phenyl]pyridine-2-carboxylic acid

### Step A. Procedure for Preparation of methyl 3-[4-(3-bromo-2-methyl-phenoxy) phenyl]propanoate

To a solution of methyl 3-(4-hydroxyphenyl)propanoate (1 g, 5.55 mmol, 1 equiv.) in NMP (10 mL) was added 1,3-dibromo-2-methyl-benzene (4.16 g, 16.65 mmol, 3 equiv.), CuI (528.44 mg, 2.77 mmol, 0.5 equiv.), Cs₂CO₃ (2.17 g, 6.66 mmol, 1.2 equiv.), and dipivaloylmethane (255.65 mg, 1.39 mmol, 285.65 μL, 0.25 equiv.) at 25° C. The reaction mixture was stirred at 120° C. for 16 hours under N₂ atmosphere. The reaction mixture was diluted with water (20 mL) and extracted with ethyl acetate 90 mL (30 mL×3). The combined organic layers were washed with brine 60 mL (20 mL×3), dried over sodium sulfate, filtered, and concentrated under reduced pressure to give a residue. The residue was purified by flash silica gel chromatography (eluent of 0-10% ethyl acetate/petroleum ether). Methyl 3-[4-(3-bromo-2-methyl-phenoxy) phenyl]propanoate (1.68 g, 4.33 mmol, 78.02% yield, 90% purity) was obtained as a yellow oil.

MS (ESI) m/z: 350.7 [M+H]⁺

¹H NMR (400 MHz, CD₃OD) δ=7.35 (d, J=7.6 Hz, 1H), 7.21-7.16 (m, 2H), 7.06 (t, J=8.0 Hz, 1H), 6.85-6.77 (m, 3H), 3.67-3.61 (m, 3H), 2.89 (t, J=7.6 Hz, 2H), 2.65-2.59 (m, 2H), 2.30 (s, 3H)

### Step B. Procedure for Preparation of tert-butyl 6-[8-(1,3-benzothiazol-2-ylcarbamoyl)-3,4-dihydro-1H-isoquinolin-2-yl]-3-[3-[4-(3-methoxy-3-oxo-propyl)phenoxy]-2-methyl-phenyl]pyridine-2-carboxylate

To a solution of methyl 3-[4-(3-bromo-2-methyl-phenoxy)phenyl]propanoate (1 g, 2.86 mmol, 1 equiv.) in dioxane (10 mL) was added tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (1.93 g, 3.15 mmol, 1.1 equiv.), Ad₂nBuP Pd G₃ (cataCXium® A Pd G₃) (417.09 mg, 572.71 mol, 0.2 equiv.), and K₃PO₄ (1.5 M, 5.73 mL, 3 equiv.) at 25° C. The reaction mixture was purged with N₂ three times and stirred under N₂ atmosphere at 80° C. for 16 hours. The reaction mixture was diluted with water (10 mL) and extracted with ethyl acetate 60 mL (20 mL×3). The combined organic layers were washed with brine 20 mL, dried over sodium sulfate, filtered, and concentrated under reduced pressure to give a residue. The residue was purified by flash silica gel chromatography (eluent of 0˜50% ethyl acetate/petroleum ether). tert-Butyl 6-[8-(1,3-benzothiazol-2-ylcarbamoyl)-3,4-dihydro-1H-isoquinolin-2-yl]-3-[3-[4-(3-methoxy-3-oxo-propyl)phenoxy]-2-methyl-phenyl]pyridine-2-carboxylate (1 g, 1.30 mmol, 45.34% yield, 98% purity) was obtained as a yellow solid.

MS (ESI) m/z: 755.3 [M+H]⁺.

### Step C. Procedure for Preparation of 3-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]phenyl]propanoic acid

To a solution of tert-butyl 6-[8-(1,3-benzothiazol-2-ylcarbamoyl)-3,4-dihydro-1H-isoquinolin-2-yl]-3-[3-[4-(3-methoxy-3-oxo-propyl)phenoxy]-2-methyl-phenyl]pyridine-2-carboxylate (500 mg, 662.35 mol, 1 equiv.) in THF (5 mL) was added LiOH·H₂O (1 M, 1.99 mL, 3 equiv.) at 25° C. The reaction mixture was stirred at 25° C. for 2 hours. The reaction mixture was acidified with 1N HCl to pH=6 and filtered to give a residue. 3-[4-[3-[6-[8-(1,3-Benzothiazol-2-ylcarbamoyl)-3,4-dihydro-1H-isoquinolin-2-yl]-2-tert-butoxycarbonyl-3-pyridyl]-2-methyl-phenoxy]phenyl]propanoic acid (616 mg, crude) was obtained as a white solid.

MS (ESI) m/z: 741.3 [M+H]⁺.

### Step D. Procedure for Preparation of tert-butyl 6-[8-(1,3-benzothiazol-2-ylcarbamoyl)-3,4-dihydro-1H-isoquinolin-2-yl]-3-[3-[4-[3-[[(1S)-1-[(2S, 4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propyl]phenoxy]-2-methyl-phenyl]pyridine-2-carboxylate

To a solution of 3-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]phenyl]propanoic acid (150 mg, 202.47 mol, 1 equiv.) in DMF (1.5 mL) was added (2S, 4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (99.02 mg, 222.71 mol, 1.1 equiv.), HATU (84.68 mg, 222.71 mol, 1.1 equiv.), and DIPEA (78.50 mg, 607.40 mol, 105.80 L, 3 equiv) at 25° C. The reaction mixture was stirred at 25° C. for 16 hours. The reaction mixture was concentrated under reduced pressure to give tert-butyl 6-[8-(1,3-benzothiazol-2-ylcarbamoyl)-3,4-dihydro-1H-isoquinolin-2-yl]-3-[3-[4-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propyl]phenoxy]-2-methyl-phenyl]pyridine-2-carboxylate (200 mg, crude) as a yellow solid.

MS (ESI) m/z: 584.5 [M+H]⁺.

### Step E. Procedure for Preparation of 6-[8-(1,3-benzothiazol-2-ylcarbamoyl)-3,4-dihydro-1H-isoquinolin-2-yl]-3-[3-[4-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propyl]phenoxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[4-[3-[[(1S)-1-[(2S,4R)-4-hydroxy-2-[[(1S)-1-[4-(4-methylthiazol-5-yl) phenyl]ethyl]carbamoyl]pyrrolidine-1-carbonyl]-2,2-dimethyl-propyl]amino]-3-oxo-propyl]phenoxy]-2-methyl-phenyl]pyridine-2-carboxylate (200 mg, 171.32 mol, 1 equiv.) in DCM (1 mL) and TFA (1 mL) was stirred at 40° C. for 2 hours. The reaction mixture was concentrated under reduced pressure to give a residue. The residue was purified by prep-HPLC. 6-[8-(1,3-Benzothiazol-2-ylcarbamoyl)-3,4-dihydro-1H-isoquinolin-2-yl]-3-[3-[4-[3-[[(1S)-1-[(2S,4R)-4-hydroxy-2-[[(1S)-1-[4-(4-methylthiazol-5-yl) phenyl]ethyl]carbamoyl]pyrrolidine-1-carbonyl]-2,2-dimethyl-propyl]amino]-3-oxo-propyl]phenoxy]-2-methyl-phenyl]pyridine-2-carboxylic acid (50.1 mg, 41.9 mol, 24.4% yield, 92.9% purity) was obtained as a yellow solid.

MS (ESI) m/z: 1111.3 [M+H]⁺.

¹H NMR (400 MHz, CD₃OD) δ=8.87-8.84 (m, 1H), 7.93-7.89 (m, 1H), 7.77 (d, J=8.0 Hz, 1H), 7.63-7.59 (m, 1H), 7.50 (d, J=8.8 Hz, 1H), 7.47-7.42 (m, 3H), 7.41 (d, J=2.4 Hz, 3H), 7.39-7.37 (m, 1H), 7.35-7.31 (m, 1H), 7.17-7.13 (m, 3H), 7.10 (d, J=8.8 Hz, 1H), 6.86 (dd, J=7.6, 14.8 Hz, 2H), 6.82-6.78 (m, 2H), 5.10 (s, 2H), 4.98 (d, J=7.2 Hz, 1H), 4.61-4.58 (m, 1H), 4.54 (t, J=8.2 Hz, 1H), 4.42 (s, 1H), 4.00 (t, J=6.0 Hz, 2H), 3.86 (d, J=11.2 Hz, 1H), 3.72 (dd, J=4.0, 10.8 Hz, 1H), 3.11 (t, J=6.0 Hz, 2H), 2.88 (t, J=7.6 Hz, 2H), 2.62-2.52 (m, 2H), 2.47-2.45 (m, 3H), 2.18 (s, 1H), 1.98-1.93 (m, 1H), 1.91 (s, 3H), 1.48 (d, J=7.2 Hz, 3H), 0.95 (s, 9H)

### Example 13. Preparation of Compound 113a

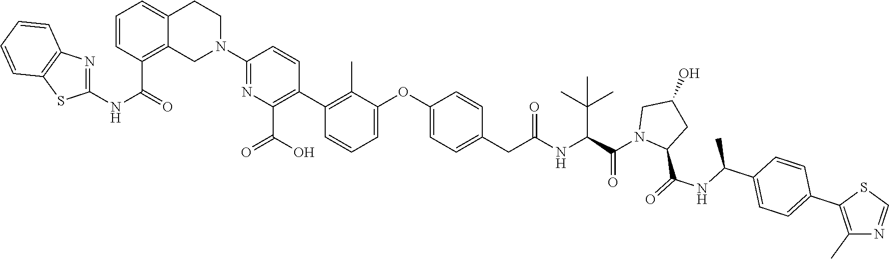 [SMILES: Cc1ncsc1-c1ccc([C@H](C)NC(=O)[C@@H]2C[C@@H](O)CN2C(=O)[C@@H](NC(=O)Cc2ccc(Oc3cccc(-c4ccc(N5CCc6cccc(C(=O)Nc7nc8ccccc8s7)c6C5)nc4C(=O)O)c3C)cc2)C(C)(C)C)cc1]

### 6-[8-(1,3-benzothiazol-2-ylcarbamoyl)-3,4-dihydro-1H-isoquinolin-2-yl]-3-[3-[4-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]phenoxy]-2-methyl-phenyl]pyridine-2-carboxylic acid

### Step A. Procedure for Preparation of methyl 2-[4-(3-bromo-2-methyl-phenoxy)phenyl]acetate

A mixture of methyl 2-(4-hydroxyphenyl)acetate (500 mg, 3.01 mmol, 1 equiv.), 1,3-dibromo-2-methyl-benzene (2.26 g, 9.03 mmol, 3 equiv.), iodocopper (286.52 mg, 1.50 mmol, 0.5 equiv.), dicesium;carbonate (1.18 g, 3.61 mmol, 1.2 equiv.), and 2,2,6,6-tetramethylheptane-3,5-dione (138.62 mg, 752.23 μmol, 154.88 μL, 0.25 equiv.) in NMP (20 mL) was stirred at 120° C. for 16 hours. The reaction mixture was concentrated under reduced pressure to give a residue. The residue was purified by flash silica gel chromatography (eluent of 2-5% ethyl acetate/petroleum ether) to give methyl 2-[4-(3-bromo-2-methyl-phenoxy)phenyl]acetate (3.2 g, 9.5 mmol, 79.3% yield) as a colorless oil.

¹H NMR (400 MHz, DMSO-d₆) δ=7.44 (d, J=7.6 Hz, 1H), 7.27-7.25 (m, 2H), 7.17 (t, J=8.0 Hz, 1H), 6.92-6.87 (m, 3H), 3.66 (s, 2H), 3.62 (s, 3H), 2.26 (s, 3H)

### Step B. Procedure for Preparation of tert-butyl 6-(8-(benzo[d]thiazol-2-ylcarbamoyl)-3,4-dihydroisoquinolin-2(1H)-yl)-3-(3-(4-(2-methoxy-2-oxoethyl)phenoxy)-2-methylphenyl)picolinate

A mixture of methyl 2-[4-(3-bromo-2-methyl-phenoxy)phenyl]acetate (250 mg, 745.84 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (639.61 mg, 1.04 mmol, 1.4 equiv.), K₂CO₃ (1.5 M, 745.84 μL, 1.5 equiv.), and [2-(2-aminophenyl)phenyl]palladium(1+);bis(1-adamantyl)-butyl-phosphane;methanesulfonate (108.63 mg, 149.17 μmol, 0.2 equiv.) in dioxane (3 mL) was degassed and purged with N₂ three times, and then the mixture was stirred at 100° C. for 1 hour under microwave. The reaction mixture was concentrated under reduced pressure to give a residue. The residue was purified by flash silica gel chromatography (eluent of 10-47% ethyl acetate/petroleum ether) to give tert-butyl 6-(8-(benzo[d]thiazol-2-ylcarbamoyl)-3,4-dihydroisoquinolin-2(1H)-yl)-3-(3-(4-(2-methoxy-2-oxoethyl)phenoxy)-2-methylphenyl)picolinate (610 mg) as a yellow oil.

MS (ESI) m/z: 741.7 [M+H]⁺.

### Step C. Procedure for Preparation of 2-(4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)phenyl)acetic acid

A mixture of tert-butyl 6-(8-(benzo[d]thiazol-2-ylcarbamoyl)-3,4-dihydroisoquinolin-2(IH)-yl)-3-(3-(4-(2-methoxy-2-oxoethyl)phenoxy)-2-methylphenyl)picolinate (500 mg, 674.89 μmol, 1 equiv.) and LiOH·H₂O (141.60 mg, 3.37 mmol, 5 equiv.) in THF (3 mL) and water (1 mL) was stirred at 40° C. for 16 hours. The reaction mixture was diluted with water (2 mL) and concentrated as a turbid liquid. Then the pH of the turbid liquid was adjusted to 3 with 1 N HCl (1 mL). A residue was obtained after trituration and filtered. The residue was used in the next step without other purification. 2-(4-(3-(6-(8-(Benzo[d]thiazol-2-ylcarbamoyl)-3,4-dihydroisoquinolin-2(1H)-yl)-2-(tert-butoxycarbonyl)pyridin-3-yl)-2-methylphenoxy)phenyl)acetic acid (395 mg, 543.4 μmol, 80.5% yield) was obtained as a white solid.

MS (ESI) m/z: 727.7 [M+H]⁺.

### Step D. Procedure for Preparation of tert-butyl 6-[8-(1,3-benzothiazol-2-ylcarbamoyl)-3,4-dihydro-1H-isoquinolin-2-yl]-3-[3-[4-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]phenoxy]-2-methyl-phenyl]pyridine-2-carboxylate

A mixture of 2-(4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)phenyl)acetic acid (80.00 mg, 110.07 μmol, 1 equiv.), (2S,4R)-1-[(2S)-2-amino-3,3-dimethyl-butanoyl]-4-hydroxy-N−[(1S)-1-[4-(4-methylthiazol-5-yl)phenyl]ethyl]pyrrolidine-2-carboxamide (73.40 mg, 165.10 μmol, 1.5 equiv.), and EDCI (52.75 mg, 275.16 μmol, 2.5 equiv.) in pyridine (1.8 mL) was stirred at 40° C. for 16 hours. The mixture was diluted with water (2 mL) and 1 N HCl 4 mL (2 mL×5) and extracted with ethyl acetate 3 mL (1 mL×3). The combined organic layers were filtered and concentrated under reduced pressure to give a residue. This residue was purified by flash silica gel chromatography (eluent of 50˜100% ethyl acetate/petroleum ether) to give tert-butyl 6-[8-(1,3-benzothiazol-2-ylcarbamoyl)-3,4-dihydro-1H-isoquinolin-2-yl]-3-[3-[4-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]phenoxy]-2-methyl-phenyl]pyridine-2-carboxylate (30 mg, 26.0 μmol, 23.6% yield) as a yellow oil.

MS (ESI) m/z: 1149.9 [M+H]⁺.

### Step E. Procedure for Preparation of 6-[8-(1,3-benzothiazol-2-ylcarbamoyl)-3,4-dihydro-1H-isoquinolin-2-yl]-3-[3-[4-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]phenoxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[4-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]phenoxy]-2-methyl-phenyl]pyridine-2-carboxylate (30 mg, 26.01 μmol, 1 equiv.) in DCM (0.5 mL) and TFA (0.5 mL) was stirred at 25° C. for 16 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[4-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]phenoxy]-2-methyl-phenyl]pyridine-2-carboxylic acid (8.4 mg, 7.5 μmol, 28.8% yield, 97.0% purity) as a white solid.

MS (ESI) m/z: 1097.8 [M+H]⁺.

¹H NMR (400 MHz, DMSO-d₆) δ=8.99 (s, 1H), 8.41 (d, J=7.6 Hz, 1H), 8.02 (d, J=7.6 Hz, 2H), 7.78 (d, J=8.0 Hz, 1H), 7.64 (d, J=7.6 Hz, 1H), 7.48-7.43 (m, 5H), 7.39-7.34 (m, 4H), 7.23 (d, J=8.4 Hz, 2H), 7.16 (t, J=7.6 Hz, 1H), 6.92 (d, J=7.2 Hz, 2H), 6.83-6.81 (m, 3H), 5.00 (s, 1H), 4.92 (t, J=7.2 Hz, 1H), 4.51-4.42 (m, 2H), 4.27 (s, 1H), 3.93 (t, J=5.2 Hz, 2H), 3.59 (d, J=5.2 Hz, 2H), 3.46-3.42 (m, 4H), 3.03 (t, J=5.2 Hz, 2H), 2.46 (s, 3H), 2.05-1.99 (m, 1H), 1.92 (s, 3H), 1.81-1.75 (m, 1H), 1.38 (d, J=7.2 Hz, 3H), 0.92-0.89 (m, 9H)

### Example 14. Preparation of Compound 114a

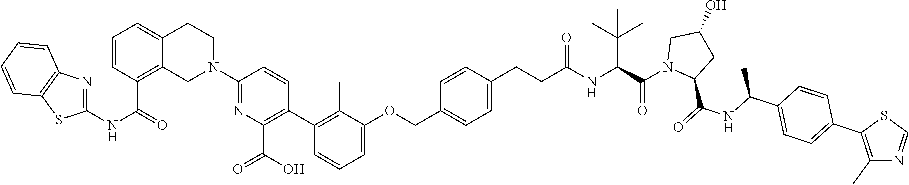 [SMILES: Cc1ncsc1-c1ccc([C@H](C)NC(=O)[C@@H]2C[C@@H](O)CN2C(=O)[C@@H](NC(=O)CCc2ccc(COc3cccc(-c4ccc(N5CCc6cccc(C(=O)Nc7nc8ccccc8s7)c6C5)nc4C(=O)O)c3C)cc2)C(C)(C)C)cc1]

### 6-(8-(benzo[d]thiazol-2-ylcarbamoyl)-3,4-dihydroisoquinolin-2(1H)-yl)-3-(3-((4-(3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropyl)benzyl)oxy)-2-methylphenyl)picolinic acid

### Step A. Procedure for Preparation of (E)-methyl 3-(4-(hydroxymethyl) phenyl) acrylate

To a solution of (4-iodophenyl)methanol (2 g, 8.55 mmol, 1 equiv.) in DMF (20 mL) was added Pd(OAc)₂ (191.86 mg, 854.58 mol, 0.1 equiv.), Et₃N (1.73 g, 17.09 mmol, 2.38 mL, 2 equiv.), tris-o-tolylphosphane (520.22 mg, 1.71 mmol, 0.2 equiv.), and methyl acrylate (820 mg, 9.53 mmol, 857.74 L, 1.11 equiv.) at 25° C. under N₂. The reaction mixture was purged with N₂ three times and stirred at 90° C. for 16 hours. The reaction mixture was cooled to room temperature and then diluted with water (20 mL) and extracted with ethyl acetate 120 mL (40 mL×3). The combined organic layers were washed with brine 90 mL (30 mL×3), dried over sodium sulfate, filtered, and concentrated under reduced pressure to give a residue. The residue was purified by flash silica gel chromatography (eluent of 0-20% ethyl acetate/petroleum ether) to give (E)-methyl 3-(4-(hydroxymethyl) phenyl)acrylate (1.6 g, 8.3 mmol, 97.4% yield) as a black solid.

¹H NMR (400 MHz, CDCl₃) δ=7.69 (d, J=16.0 Hz, 1H), 7.52 (d, J=8.0 Hz, 2H), 7.39 (d, J=8.0 Hz, 2H), 6.44 (d, J=16.0 Hz, 1H), 4.73 (s, 2H), 3.81 (s, 3H)

### Step B. Procedure for Preparation of methyl 3-(4-(hydroxymethyl)phenyl)propanoate

A mixture of (E)-methyl 3-(4-(hydroxymethyl)phenyl)acrylate (1.6 g, 8.32 mmol, 1 equiv.) and chloro rhodium;triphenylphosphane (770.17 mg, 832.42 mol, 0.1 equiv.) in MeOH (20 mL) was degassed and purged with N₂ three times, and then the mixture was stirred at 25° C. for 24 hours under H₂ (50 Psi) atmosphere. The mixture was filtered and concentrated under reduced pressure to give methyl 3-(4-(hydroxymethyl)phenyl)propanoate (1.6 g, crude) as a yellow oil.

¹H NMR (400 MHz, CDCl₃) δ=7.30 (d, J=8.0 Hz, 2H), 7.23-7.19 (m, 2H), 4.67 (s, 2H), 3.68 (s, 3H), 2.98-2.94 (m, 2H), 2.64 (t, J=7.6 Hz, 2H)

### Step C. Procedure for Preparation of methyl 3-(4-((3-bromo-2-methylphenoxy)methyl)phenyl)propanoate

To a solution of methyl 3-(4-(hydroxymethyl)phenyl)propanoate (950 mg, 4.89 mmol, 1 equiv.) in THF (10 mL) was added 3-bromo-2-methyl-phenol (914.82 mg, 4.89 mmol, 1 equiv.), DIAD (1.58 g, 7.83 mmol, 1.52 mL, 1.6 equiv.), and PPh₃ (2.05 g, 7.83 mmol, 1.6 equiv.) at 0° C. The reaction mixture was stirred at 25° C. for 2 hours. The reaction mixture was filtered and concentrated under reduced pressure to give a residue. The residue was purified by flash silica gel chromatography (eluent of 0˜10% ethyl acetate/petroleum ether) to give methyl 3-(4-((3-bromo-2-methylphenoxy)methyl)phenyl)propanoate (1.4 g, 3.9 mmol, 81.6% yield) as a yellow oil.

¹H NMR (400 MHz, CDCl₃) δ=7.36 (d, J=8.0 Hz, 2H), 7.23 (d, J=8.0 Hz, 2H), 7.18 (d, J=8.0 Hz, 1H), 7.00 (t, J=8.0 Hz, 1H), 6.84 (d, J=8.4 Hz, 1H), 5.04 (s, 2H), 3.69 (s, 3H), 2.98 (t, J=7.6 Hz, 2H), 2.66 (t, J=7.6 Hz, 2H), 2.37 (s, 3H)

### Step D. Procedure for Preparation of tert-butyl 6-(8-(benzo[d]thiazol-2-ylcarbamoyl)-3,4-dihydroisoquinolin-2(1H)-yl)-3-(3-((4-(3-methoxy-3-oxopropyl)benzyl)oxy)-2-methylphenyl)picolinate

methyl 3-(4-((3-bromo-2-methylphenoxy)methyl)phenyl)propanoate (500 mg, 1.38 mmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (843.16 mg, 1.38 mmol, 1 equiv.), KF (1.5 M, 2.75 mL, 3 equiv.), and [2-(2-aminophenyl)phenyl]palladium(1+);bis(1-adamantyl)-butyl-phosphane;methanesulfonate (200.49 mg, 275.30 μmol, 0.2 equiv.) were taken up into a microwave tube in 1,4-dioxane (5 mL). The sealed tube was heated at 100° C. for 1 hour under microwave. The reaction mixture was concentrated under reduced pressure to give a residue. The residue was purified by flash silica gel chromatography (eluent of 0˜30% ethyl acetate/petroleum ether) to give tert-butyl 6-(8-(benzo[d]thiazol-2-ylcarbamoyl)-3,4-dihydroisoquinolin-2(1H)-yl)-3-(3-((4-(3-methoxy-3-oxopropyl)benzyl)oxy)-2-methylphenyl)picolinate (640 mg, 626.7 μmol, 45.5% yield, 75.3% purity) was obtained as a yellow solid.

MS (ESI) m/z: 769.8 [M+H]⁺.

### Step E. Procedure for Preparation of 3-[4-[[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]methyl]phenyl]propanoic acid

To a solution of tert-butyl 6-(8-(benzo[d]thiazol-2-ylcarbamoyl)-3,4-dihydroisoquinolin-2(1H)-yl)-3-(3-((4-(3-methoxy-3-oxopropyl)benzyl)oxy)-2-methylphenyl)picolinate (640 mg, 832.34 μmol, 1 equiv.) in THF (6 mL) and water (1 mL) was added LiOH·H₂O (104.78 mg, 2.50 mmol, 3 equiv.) at 25° C. The reaction mixture was stirred at 40° C. for 16 hours. The reaction mixture was concentrated under reduced pressure to give a residue. The residue was treated with water (6 mL). The pH was adjusted to around 3 by progressively adding diluted HCl. The mixture was filtered to give a residue. 3-[4-[[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]methyl]phenyl]propanoic acid (600 mg, 725.6 μmol, 87.1% yield, 91.3% purity) was obtained as a yellow solid.

MS (ESI) m/z: 755.7 [M+H]⁺.

### Step F. Procedure for Preparation of tert-butyl 6-[8-(1,3-benzothiazol-2-ylcarbamoyl)-3,4-dihydro-1H-isoquinolin-2-yl]-3-[3-[[4-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propyl]phenyl]methoxy]-2-methyl-phenyl]pyridine-2-carboxylate

A mixture of 3-[4-[[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]methyl]phenyl]propanoic acid (120 mg, 158.96 μmol, 1 equiv.) and (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (84.81 mg, 190.76 μmol, 1.2 equiv.) in pyridine (1.2 mL) was added EDCI (36.57 mg, 190.76 μmol, 1.2 equiv.). The mixture was stirred at 25° C. for 24 hours. The reaction mixture was added into water (5 mL). The mixture was triturated with water at 25° C. for 0.5 hours and filtered to give compound tert-butyl 6-[8-(1,3-benzothiazol-2-ylcarbamoyl)-3,4-dihydro-1H-isoquinolin-2-yl]-3-[3-[[4-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propyl]phenyl]methoxy]-2-methyl-phenyl]pyridine-2-carboxylate (168 mg, 105.7 μmol, 66.5% yield, 74.4% purity) as a white solid.

MS (ESI) m/z: 1181.9 [M+H]⁺.

### Step G. Procedure for Preparation of 6-(8-(benzo[d]thiazol-2-ylcarbamoyl)-3,4-dihydroisoquinolin-2(1H)-yl)-3-(3-((4-(3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropyl)benzyl)oxy)-2-methylphenyl)picolinic acid

To a solution of tert-butyl 6-[8-(1,3-benzothiazol-2-ylcarbamoyl)-3,4-dihydro-1H-isoquinolin-2-yl]-3-[3-[[4-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propyl]phenyl]methoxy]-2-methyl-phenyl]pyridine-2-carboxylate (138 mg, 116.80 μmol, 1 equiv.) in CH₂Cl₂ (0.7 mL) was added TFA (1.08 g, 9.45 mmol, 0.7 mL, 80.94 equiv.). The mixture was stirred at 25° C. for 16 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-((4-(3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropyl)benzyl)oxy)-2-methylphenyl)picolinic acid (13.3 mg, 11.0 μmol, 9.4% yield, 93.4% purity) was obtained as a yellow solid.

MS (ESI) m/z: 1125.8[M+H]⁺.

¹H NMR (400 MHz, DMSO-d₆) δ=9.00-8.95 (m, 1H), 8.32-8.37 (m, 1H), 7.98-8.06 (m, 1H), 7.87-7.91 (m, 1H), 7.78-7.83 (m, 1H), 7.66-7.59 (m, 1H), 7.47-7.41 (m, 5H), 7.37-7.39 (m, 6H), 7.24 (d, J=8.0 Hz, 2H), 7.11 (t, J=7.6 Hz, 1H), 7.01-6.93 (m, 2H), 6.66 (d, J=8.0 Hz, 1H), 5.14-5.08 (m, 1H), 5.05 (s, 2H), 4.98 (s, 2H), 4.91 (t, J=7.2 Hz, 1H), 4.51 (d, J=9.6 Hz, 1H), 4.42 (t, J=8.0 Hz, 1H), 4.31-4.24 (m, 1H), 3.94-3.88 (m, 2H), 3.60 (s, 2H), 3.02 (t, J=6.0 Hz, 2H), 2.87-2.75 (m, 3H), 2.45 (s, 3H), 2.04-1.97 (m, 1H), 1.92 (s, 3H), 1.84-1.74 (m, 1H), 1.37 (d, J=7.2 Hz, 3H), 1.27-1.20 (m, 1H), 0.88 (s, 9H)

### Example 15. Preparation of Compound 115a

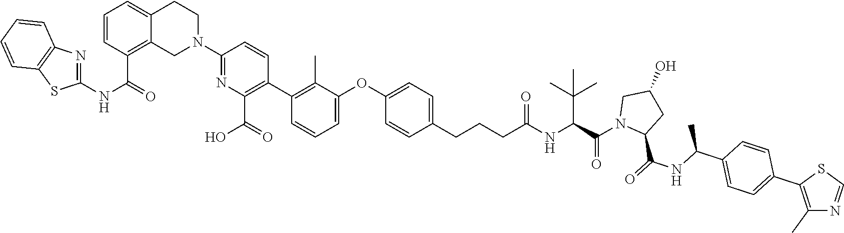 [SMILES: Cc1ncsc1-c1ccc([C@H](C)NC(=O)[C@@H]2C[C@@H](O)CN2C(=O)[C@@H](NC(=O)CCCc2ccc(Oc3cccc(-c4ccc(N5CCc6cccc(C(=O)Nc7nc8ccccc8s7)c6C5)nc4C(=O)O)c3C)cc2)C(C)(C)C)cc1]

### 6-(8-(benzo[d]thiazol-2-ylcarbamoyl)-3,4-dihydroisoquinolin-2 (1H)-yl)-3-(3-(4-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)phenoxy)-2-methylphenyl)picolinic acid

### Step A. Procedure for Preparation of tert-butyl 6-(8-(benzo[d]thiazol-2-ylcarbamoyl)-3,4-dihydroisoquinolin-2 (1H)-yl)-3-(3-(4-(4-methoxy-4-oxobutyl)phenoxy)-2-methylphenyl)picolinate

To a solution of tert-butyl 6-(8-(benzo[d]thiazol-2-ylcarbamoyl)-3,4-dihydroisoquinolin-2 (1H)-yl)-3-(4,4,5,5-tetramethyl-1,3,2-dioxaborolan-2-yl)picolinate (200 mg, 326.51 μmol, 1 equiv.) in dioxane (2 mL) and water (0.5 mL) was added [2-(2-aminophenyl)phenyl]palladium(1+);bis(1-adamantyl)-butyl-phosphane;methane sulfonate (23.78 mg, 32.65 μmol, 0.1 equiv.), KF (56.91 mg, 979.52 μmol, 22.95 μL, 3 equiv.), and methyl 4-(4-bromophenyl)butanoate (118.60 mg, 326.51 μmol, 1 equiv.). The mixture was stirred at 100° C. for 1 hour. The reaction mixture was concentrated under reduced pressure to remove solvent. The residue was purified by flash silica gel chromatography (eluent of 0˜36% ethyl acetate/petroleum ether) to give tert-butyl 6-(8-(benzo[d]thiazol-2-ylcarbamoyl)-3,4-dihydroisoquinolin-2 (1H)-yl)-3-(3-(4-(4-methoxy-4-oxobutyl)phenoxy)-2-methylphenyl)picolinate (151 mg, 196.3 μmol, 60.1% yield) as a yellow solid.

MS (ESI) m/z: 769.8 [M+H]⁺.

### Step B. Procedure for Preparation of 4-(4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)phenyl)butanoic acid

To a solution of tert-butyl 6-(8-(benzo[d]thiazol-2-ylcarbamoyl)-3,4-dihydroisoquinolin-2 (1H)-yl)-3-(3-(4-(4-methoxy-4-oxobutyl)phenoxy)-2-methylphenyl)picolinate (150 mg, 195.08 μmol, 1 equiv.) in THF (1.5 mL) was added LiOH·H₂O (24.56 mg, 585.24 μmol, 3 equiv.) and water (0.4 mL). The mixture was stirred at 25° C. for 12 hours. The reaction mixture was acidified to pH=5-6 with citric acid, filtered, and concentrated under reduced pressure to give a residue. The crude product was used in the next step without further purification. 4-(4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)phenyl)butanoic acid (129 mg, 170.8 μmol, 87.6% yield) was obtained as a white solid.

MS (ESI) m/z: 755.5 [M+H]⁺.

¹H NMR (400 MHz, DMSO) δ=12.86 (s, 1H), 9.66 (t, J=1.6 Hz, 1H), 8.03 (d, J=7.6 Hz, 1H), 7.79 (d, J=8.0 Hz, 1H), 7.61 (d, J=7.6 Hz, 1H), 7.55-7.43 (m, 3H), 7.42-7.32 (m, 2H), 7.17 (d, J=8.4 Hz, 3H), 6.98 (d, J=8.8 Hz, 1H), 6.88-6.79 (m, 4H), 5.00 (s, 2H), 3.94-3.83 (m, 2H), 3.05 (t, J=5.60 Hz, 2H), 2.57-2.53 (m, 2H), 2.43 (dt, J=1.6, 7.2 Hz, 2H), 1.91 (s, 3H), 1.81 (q, J=7.6 Hz, 2H), 1.07 (s, 9H)

### Step C. Procedure for Preparation of tert-butyl 3-(3-(4-(4-(((S)-1-((2S, 4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)phenoxy)-2-methylphenyl)-6-(8-((4-methylthiazol-2-yl)carbamoyl)-3,4-dihydroisoquinolin-2 (1H)-yl)picolinate

To a solution of 4-(4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)phenyl)butanoic acid (100 mg, 132.47 μmol, 1 equiv.) in DMF (2 mL) was added DIEA (51.36 mg, 397.41 μmol, 69.22 μL, 3 equiv.), HATU (65.48 mg, 172.21 μmol, 1.3 equiv.) and (2S, 4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (64.78 mg, 145.72 μmol, 1.1 equiv.). The mixture was stirred at 25° C. for 1 hour. The reaction was diluted with water (20 mL) and extracted with DCM 100 mL (50 mL×2). The combined organic layers were washed with brine 50 mL, dried over sodium sulfate, filtered, and concentrated under reduced pressure to give tert-butyl 3-(3-(4-(4-(((S)-1-((2S, 4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)phenoxy)-2-methylphenyl)-6-(8-((4-methylthiazol-2-yl)carbamoyl)-3,4-dihydroisoquinolin-2 (1H)-yl)picolinate (120 mg, 101.5 μmol, 76.7% yield) as a yellow oil.

MS (ESI) m/z: 1182.4 [M+H]⁺.

### Step D. Procedure for Preparation of 6-(8-(benzo[d]thiazol-2-ylcarbamoyl)-3,4-dihydroisoquinolin-2 (1H)-yl)-3-(3-(4-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)phenoxy)-2-methylphenyl)picolinic acid

To a solution of tert-butyl 3-(3-(4-(4-(((S)-1-((2S, 4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)phenoxy)-2-methylphenyl)-6-(8-((4-methylthiazol-2-yl)carbamoyl)-3,4-dihydroisoquinolin-2 (1H)-yl)picolinate (120 mg, 101.57 μmol, 1 equiv.) in DCM (2 mL) was added TFA (1 mL). The mixture was stirred at 25° C. for 12 hours. The reaction mixture was concentrated under reduced pressure to remove solvent. The residue was purified by prep-HPLC to give 6-(8-(benzo[d]thiazol-2-ylcarbamoyl)-3,4-dihydroisoquinolin-2 (1H)-yl)-3-(3-(4-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)phenoxy)-2-methylphenyl)picolinic acid (27.4 mg, 21.3 μmol, 21.0% yield, 87.5% purity) as a yellow solid.

MS (ESI) m/z: 1125.8 [M+H]⁺.

¹H NMR (400 MHz, DMSO-d₆) δ=12.42 (s, 2H), 9.01-8.94 (m, 1H), 8.38 (d, J=7.6 Hz, 1H), 8.15 (s, 1H), 8.04 (d, J=8.0 Hz, 1H), 7.89-7.77 (m, 2H), 7.63 (d, J=7.6 Hz, 1H), 7.56-7.34 (m, 9H), 7.22-7.11 (m, 3H), 7.00 (d, J=9.2 Hz, 1H), 6.91-6.78 (m, 4H), 5.10 (s, 1H), 5.00 (s, 2H), 4.96-4.86 (m, 1H), 4.54 (d, J=9.2 Hz, 1H), 4.46-4.39 (m, 1H), 4.31-4.25 (m, 1H), 3.99-3.88 (m, 2H), 3.67-3.58 (m, 2H), 3.08-2.99 (m, 2H), 2.55-2.52 (m, 4H), 2.47-2.42 (m, 3H), 2.31-2.23 (m, 1H), 2.20-2.12 (m, 1H), 2.04-1.97 (m, 1H), 1.92 (s, 3H), 1.80-1.73 (m, 2H), 1.37 (d, J=7.2 Hz, 2H), 0.95 (s, 8H)

### Example 16. Preparation of Compound 116a

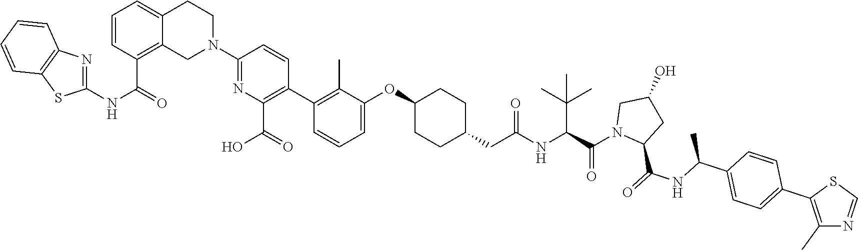 [SMILES: Cc1ncsc1-c1ccc([C@H](C)NC(=O)[C@@H]2C[C@@H](O)CN2C(=O)[C@@H](NC(=O)C[C@H]2CC[C@H](Oc3cccc(-c4ccc(N5CCc6cccc(C(=O)Nc7nc8ccccc8s7)c6C5)nc4C(=O)O)c3C)CC2)C(C)(C)C)cc1]

### 6-(8-(benzo[d]thiazol-2-ylcarbamoyl)-3,4-dihydroisoquinolin-2 (1H)-yl)-3-(3-(((1S,4r)-4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)oxy)-2-methylphenyl)picolinic acid

### Step A. Procedure for Preparation of methyl 2-(4-hydroxycyclohexyl)acetate

To a solution of 2-(4-hydroxycyclohexyl) acetic acid (2 g, 12.64 mmol, 1.0 equiv.) in MeOH (20 mL) was added H₂SO₄ (124.00 mg, 1.26 mmol, 67.39 μL, 0.1 equiv.). The mixture was stirred at 70° C. for 3 hours under N₂ atmosphere. The reaction was diluted with water (20 mL) and extracted with DCM 50 mL (50 mL×2). The combined organic layers were washed with brine 50 mL, dried over sodium sulfate, filtered, and concentrated under reduced pressure to give a residue. The residue was used in the next step without further purification. Methyl 2-(4-hydroxycyclohexyl)acetate (2 g, 11.6 mmol, 91.8% yield) was obtained as a white oil.

### Step B. Procedure for Preparation of methyl 2-(4-(3-bromo-2-methylphenoxy)cyclohexyl)acetate

To a solution of methyl 2-(4-hydroxycyclohexyl)acetate (2 g, 11.61 mmol, 1.0 equiv.) in toluene (20 mL) was added 2-(tributyl-λ5-phosphanylidene)acetonitrile (4.20 g, 17.42 mmol, 1.5 equiv.) and 3-bromo-2-methylphenol (2.61 g, 13.94 mmol, 1.2 equiv.). The mixture was degassed and purged with N₂ and stirred at 120° C. for 2 hours under N₂ atmosphere. The reaction mixture was concentrated under reduced pressure to remove solvent. The residue was purified by flash silica gel chromatography (eluent of 0˜15% ethyl acetate/petroleum ether) to give methyl 2-(4-(3-bromo-2-methylphenoxy)cyclohexyl)acetate (3.8 g, 10.9 mmol, 94.0% yield, 98.0% purity) as a white oil.

¹H NMR (400 MHz, CDCl₃-d) δ=7.08-7.03 (m, 1H), 6.89 (t, J=8.0 Hz, 1H), 6.75-6.66 (m, 1H), 4.47 (s, 1H), 3.61 (s, 3H), 2.28-2.15 (m, 5H), 2.09-1.93 (m, 2H), 1.83-1.77 (m, 1H), 1.51 (d, J=13.2 Hz, 4H), 1.44-1.32 (m, 2H)

### Step C. Procedure for Preparation of methyl 2-((1r,4r)-4-(3-bromo-2-methylphenoxy) cyclohexyl)acetate

The methyl 2-(4-(3-bromo-2-methylphenoxy) cyclohexyl) acetate residue was purified by prep-HPLC to give methyl 2-((1r,4r)-4-(3-bromo-2-methylphenoxy) cyclohexyl) acetate (200 mg, 586.10 μmol, 11.11% yield) as a yellow oil.

¹H NMR (400 MHz, CDCl₃-d) δ=7.06 (d, J=8.0 Hz, 1H), 6.89 (t, J=8.0 Hz, 1H), 6.72 (d, J=8.0 Hz, 1H), 4.02 (tt, J=4.4, 10.6 Hz, 1H), 3.61 (s, 3H), 2.22 (s, 3H), 2.17 (d, J=6.4 Hz, 2H), 2.11-2.01 (m, 2H), 1.82-1.77 (m, 2H), 1.54 (s, 1H), 1.48-1.37 (m, 2H), 1.10-0.99 (m, 2H)

### Step D. Procedure for Preparation of tert-butyl 6-(8-(benzo[d]thiazol-2-ylcarbamoyl)-3,4-dihydroisoquinolin-2 (1H)-yl)-3-(3-(((1r,4r)-4-(2-methoxy-2-oxoethyl)cyclohexyl)oxy)-2-methylphenyl)picolinate

To a solution of tert-butyl 6-(8-(benzo[d]thiazol-2-ylcarbamoyl)-3,4-dihydroisoquinolin-2 (1H)-yl)-3-(4,4,5,5-tetramethyl-1,3,2-dioxaborolan-2-yl) picolinate (195.82 mg, 319.69 μmol, 1.0 equiv.) in dioxane (2 mL) was added Ad₂nBuP Pd G₃ (cataCXium® A Pd G₃) (23.28 mg, 31.97 μmol, 0.1 equiv.), KF (1.5 M, 639.38 μL, 3.0 equiv.), and methyl 2-((1r,4r)-4-(3-bromo-2-methylphenoxy)cyclohexyl)acetate (120 mg, 351.66 μmol, 1.1 equiv.). The mixture was stirred at 100° C. for 1 hour. The reaction mixture was filtered and concentrated under reduced pressure to give a residue. The residue was purified by flash silica gel chromatography (eluent of 0˜30% ethyl acetate/petroleum ether) to give tert-butyl 6-(8-(benzo[d]thiazol-2-ylcarbamoyl)-3,4-dihydroisoquinolin-2 (1H)-yl)-3-(3-(((1r,4r)-4-(2-methoxy-2-oxoethyl)cyclohexyl)oxy)-2-methylphenyl)picolinate (200 mg, 267.7 μmol, 83.7% yield) as a yellow oil.

MS (ESI) m/z: 747.8 [M+H]⁺.

### Step E. Procedure for Preparation of 2-((1r,4r)-4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)cyclohexyl)acetic acid

To a solution of tert-butyl 6-(8-(benzo[d]thiazol-2-ylcarbamoyl)-3,4-dihydroisoquinolin-2 (1H)-yl)-3-(3-(((1r,4r)-4-(2-methoxy-2-oxoethyl)cyclohexyl)oxy)-2-methylphenyl)picolinate (190 mg, 254.38 μmol, 1.0 equiv.) in THE (2 mL) was added LiOH·H₂O (32.02 mg, 763.14 μmol, 3.0 equiv.) and water (0.5 mL). The mixture was stirred at 25° C. for 12 hours. The reaction mixture was acidified to pH=5-6 with citric acid, filtered, and concentrated under reduced pressure to give a residue. The residue was used in the next step without further purification. 2-((1r,4r)-4-(3-(6-(8-(Benzo[d]thiazol-2-ylcarbamoyl)-3,4-dihydroisoquinolin-2 (1H)-yl)-2-(tert-butoxycarbonyl)pyridin-3-yl)-2-methylphenoxy)cyclohexyl)acetic acid (150 mg, 204.6 μmol, 80.4% yield) was obtained as a yellow solid.

MS (ESI) m/z: 733.4 [M+H]⁺.

### Step F. Procedure for Preparation of tert-butyl 6-(8-(benzo[d]thiazol-2-ylcarbamoyl)-3,4-dihydroisoquinolin-2 (1H)-yl)-3-(3-(((1S,4r)-4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)oxy)-2-methylphenyl)picolinate

To a solution of 2-((1r,4r)-4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)cyclohexyl)acetic acid (100 mg, 136.45 μmol, 1.0 equiv.) in pyridine (1 mL) was added EDCI (34.00 mg, 177.38 μmol, 1.3 equiv.) and (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (66.73 mg, 150.09 μmol, 1.1 equiv.). The mixture was stirred at 40° C. for 1 hour. The reaction mixture was concentrated under reduced pressure to remove solvent. The residue was purified by prep-TLC (SiO₂, petroleum ether/ethyl acetate=0/1) to give tert-butyl 6-(8-(benzo[d]thiazol-2-ylcarbamoyl)-3,4-dihydroisoquinolin-2 (1H)-yl)-3-(3-(((1S,4r)-4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)oxy)-2-methylphenyl)picolinate (60 mg, 51.7 μmol, 37.9% yield) as a white solid.

MS (ESI) m/z: 580.6 [M+H/2]⁺.

### Step G. Procedure for Preparation of 6-(8-(benzo[d]thiazol-2-ylcarbamoyl)-3,4-dihydroisoquinolin-2 (1H)-yl)-3-(3-(((1S,4r)-4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2 (1H)-yl)-3-(3-(((1S,4r)-4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)oxy)-2-methylphenyl)picolinate (60 mg, 43.90 μmol, 84.84% purity, 1.1 equiv.) in DCM (1 mL) was added TFA (5.01 mg, 43.90 μmol, 3.25 μL, 1.0 equiv.). The mixture was stirred at 40° C. for 4 hours. The reaction mixture was concentrated under reduced pressure to remove solvent. The residue was purified by prep-HPLC to give 6-(8-(benzo[d]thiazol-2-ylcarbamoyl)-3,4-dihydroisoquinolin-2 (1H)-yl)-3-(3-(((1S,4r)-4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)oxy)-2-methylphenyl)picolinic acid (24.4 mg, 21.6 μmol, 49.2% yield, 97.6% purity) as a yellow solid.

MS (ESI) m/z: 1103.5 [M+H]⁺.

¹H NMR (400 MHz, DMSO-d₆) δ=8.99 (s, 1H), 8.39 (d, J=7.6 Hz, 1H), 7.98 (d, J=8.0 Hz, 1H), 7.82 (d, J=8.8 Hz, 1H), 7.75 (d, J=8.0 Hz, 1H), 7.65 (d, J=7.2 Hz, 1H), 7.47-7.29 (m, 9H), 7.07-6.99 (m, 1H), 6.86-6.86 (m, 1H), 6.88 (d, J=8.0 Hz, 1H), 6.80 (d, J=7.6 Hz, 1H), 6.68 (d, J=7.6 Hz, 1H), 4.98 (s, 2H), 4.95-4.89 (m, 1H), 4.56-4.50 (m, 1H), 4.43 (t, J=7.6 Hz, 1H), 4.28 (s, 1H), 4.20-4.12 (m, 1H), 3.90 (t, J=5.2 Hz, 2H), 3.62 (s, 2H), 3.01 (t, J=5.2 Hz, 2H), 2.46 (s, 3H), 2.23-2.11 (m, 2H), 2.10-1.95 (m, 4H), 1.89 (s, 3H), 1.83-1.70 (m, 4H), 1.38 (d, J=7.2 Hz, 3H), 1.36-1.29 (m, 2H), 1.14-1.06 (m, 1H), 1.06-1.06 (m, 1H), 0.95 (s, 9H)

### Example 17. Preparation of Compound 116b

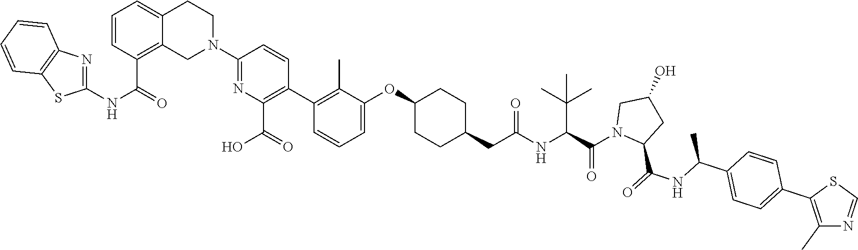 [SMILES: Cc1ncsc1-c1ccc([C@H](C)NC(=O)[C@@H]2C[C@@H](O)CN2C(=O)[C@@H](NC(=O)C[C@H]2CC[C@@H](Oc3cccc(-c4ccc(N5CCc6cccc(C(=O)Nc7nc8ccccc8s7)c6C5)nc4C(=O)O)c3C)CC2)C(C)(C)C)cc1]

### 6-(8-(benzo[d]thiazol-2-ylcarbamoyl)-3,4-dihydroisoquinolin-2 (1H)-yl)-3-(3-(((1R,4s)-4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2 (1H)-yl)-3-(3-(((1s,4s)-4-(2-methoxy-2-oxoethyl)cyclohexyl)oxy)-2-methylphenyl)picolinate

To a solution of methyl 2-((1s,4s)-4-(3-bromo-2-methylphenoxy)cyclohexyl)acetate (600 mg, 1.76 mmol, 1.0 equiv.) in dioxane (6 mL) was added (2-(2-aminophenyl)phenyl)palladium(1+); bis(1-adamantyl)-butyl-phosphane; methanesulfonate (128.05 mg, 175.83 μmol, 0.1 equiv.), tert-butyl 6-(8-(benzo[d]thiazol-2-ylcarbamoyl)-3,4-dihydroisoquinolin-2 (1H)-yl)-3-(4,4,5,5-tetramethyl-1,3,2-dioxaborolan-2-yl)picolinate (1.08 g, 1.76 mmol, 1.0 equiv.), water (2 mL) and KF (306.47 mg, 5.27 mmol, 123.58 μL, 3.0 equiv.). The mixture was stirred at 100° C. for 1 hour. The reaction mixture was concentrated under reduced pressure to remove solvent. The residue was purified by flash silica gel chromatography (eluent of 0˜25% ethyl acetate/petroleum ether) to give tert-butyl 6-(8-(benzo[d]thiazol-2-ylcarbamoyl)-3,4-dihydroisoquinolin-2 (1H)-yl)-3-(3-(((1s,4s)-4-(2-methoxy-2-oxoethyl)cyclohexyl)oxy)-2-methylphenyl)picolinate (900 mg, 1.2 mmol, 68.5% yield) as a yellow oil.

MS (ESI) m/z: 747.4 [M+H]⁺

### Step B. Procedure for Preparation of 2-((1s,4s)-4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)cyclohexyl)acetic acid

To a solution of tert-butyl 6-(8-(benzo[d]thiazol-2-ylcarbamoyl)-3,4-dihydroisoquinolin-2 (1H)-yl)-3-(3-(((1s,4s)-4-(2-methoxy-2-oxoethyl)cyclohexyl)oxy)-2-methylphenyl)picolinate (900 mg, 1.20 mmol, 1.0 equiv.) in THE (9 mL) was added LiOH·H₂O (151.68 mg, 3.61 mmol, 3.0 equiv.) and water (3 mL). The mixture was stirred at 25° C. for 1 hour. The reaction mixture was acidified to pH=5-6 with citric acid, filtered, and concentrated under reduced pressure to give a residue. The crude product was carried into next step without further purification. 2-((1s,4s)-4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)cyclohexyl)acetic acid (800 mg, 1.0 mmol, 90.5% yield) was obtained as a yellow solid.

MS (ESI) m/z: 733.1 [M+H]⁺

### Step C. Procedure for Preparation of tert-butyl 6-(8-(benzo[d]thiazol-2-ylcarbamoyl)-3,4-dihydroisoquinolin-2 (1H)-yl)-3-(3-(((1R,4s)-4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)oxy)-2-methylphenyl)picolinate

To a solution of 2-((1s,4s)-4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)cyclohexyl)acetic acid (100 mg, 136.45 μmol, 1.0 equiv.) in pyridine (1 mL) was added EDCI (34.00 mg, 177.38 μmol, 1.3 equiv.) and (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (66.73 mg, 150.09 μmol, 1.1 equiv.). The mixture was stirred at 40° C. for 1 hour. The reaction mixture was concentrated under reduced pressure to remove solvent. The residue was purified by column chromatography (SiO₂, petroleum ether/ethyl acetate=4/1 to DCM:MeOH=20:1) to give tert-butyl 6-(8-(benzo[d]thiazol-2-ylcarbamoyl)-3,4-dihydroisoquinolin-2 (1H)-yl)-3-(3-(((1R,4s)-4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)oxy)-2-methylphenyl)picolinate (140 mg, 120.7 μmol, 88.4% yield) as a yellow oil.

MS (ESI) m/z: 1160.6 [M+H]⁺

### Step D. Procedure for Preparation of 6-(8-(benzo[d]thiazol-2-ylcarbamoyl)-3,4-dihydroisoquinolin-2 (1H)-yl)-3-(3-(((1R,4s)-4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2 (1H)-yl)-3-(3-(((1R,4s)-4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)oxy)-2-methylphenyl)picolinate (140 mg, 120.75 μmol, 1.0 equiv.) in DCM (2 mL) was added TFA (13.77 mg, 120.75 μmol, 8.94 μL, 1.0 equiv.). The mixture was stirred at 25° C. for 12 hours. The reaction mixture was concentrated under reduced pressure to remove solvent. The residue was purified by prep-HPLC to give 6-(8-(benzo[d]thiazol-2-ylcarbamoyl)-3,4-dihydroisoquinolin-2 (1H)-yl)-3-(3-(((1R,4s)-4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)oxy)-2-methylphenyl)picolinic acid (8.5 mg, 7.2 μmol, 6.0% yield) as a yellow solid.

MS (ESI) m/z: 552.7 [M12+H]⁺

¹H NMR (400 MHz, DMSO-d₆) δ=13.07-12.34 (m, 2H), 8.99 (s, 1H), 8.37 (d, J=8.0 Hz, 1H), 8.04 (d, J=7.6 Hz, 1H), 7.89-7.77 (m, 2H), 7.63 (d, J=7.6 Hz, 1H), 7.48-7.42 (m, 5H), 7.40-7.32 (m, 4H), 7.11-7.05 (m, 1H), 6.98 (d, J=8.8 Hz, 1H), 6.88 (d, J=8.4 Hz, 1H), 6.61 (d, J=7.6 Hz, 1H), 5.08 (d, J=3.6 Hz, 1H), 4.99 (s, 2H), 4.91 (t, J=7.2 Hz, 1H), 4.57 (s, 1H), 4.52 (d, J=9.6 Hz, 1H), 4.42 (t, J=8.0 Hz, 1H), 4.28 (s, 1H), 3.93 (t, J=6.0 Hz, 2H), 3.61 (s, 2H), 3.11 (q, J=7.2 Hz, 3H), 3.04 (t, J=5.6 Hz, 2H), 2.97-2.90 (m, 1H), 2.46 (s, 3H), 2.21 (d, J=7.2 Hz, 1H), 2.12-2.07 (m, 1H), 1.95 (s, 3H), 1.80 (td, J=4.4, 8.5 Hz, 2H), 1.60-1.53 (m, 2H), 1.50-1.45 (m, 2H), 1.36 (s, 2H), 1.24 (s, 2H), 0.94 (s, 9H)

### Example 18. Preparation of Compound 117a

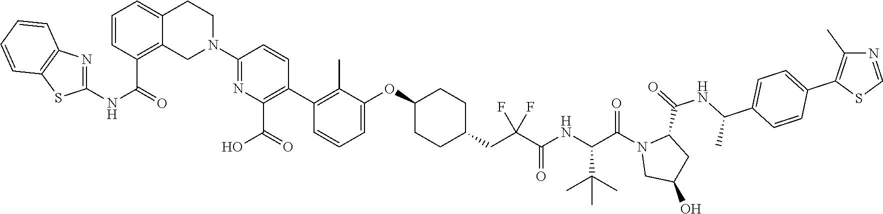 [SMILES: Cc1ncsc1-c1ccc([C@H](C)NC(=O)[C@@H]2C[C@@H](O)CN2C(=O)[C@@H](NC(=O)C(F)(F)C[C@H]2CC[C@H](Oc3cccc(-c4ccc(N5CCc6cccc(C(=O)Nc7nc8ccccc8s7)c6C5)nc4C(=O)O)c3C)CC2)C(C)(C)C)cc1]

### 6-(8-(benzo[d]thiazol-2-ylcarbamoyl)-3,4-dihydroisoquinolin-2 (1H)-yl)-3-(3-(((1S,4r)-4-(2,2-difluoro-3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropyl)cyclohexyl)oxy)-2-methylphenyl)picolinic acid

### Step A. Procedure for Preparation of methyl 4-(3-bromo-2-methyl-phenoxy)cyclohexanecarboxylate

To a solution of 3-bromo-2-methyl-phenol (30 g, 160.40 mmol, 1 equiv.) in toluene (300 mL) was added methyl 4-hydroxycyclohexanecarboxylate (27.91 g, 176.44 mmol, 1.1 equiv.) and 2-(tributyl-λ⁵-phosphanylidene)acetonitrile (46.46 g, 192.48 mmol, 1.2 equiv.) at 25° C. The reaction mixture was degassed and purged with N₂ three times, and then the mixture was stirred at 120° C. for 16 hours under N₂ atmosphere. The reaction mixture was filtered and concentrated under reduced pressure to give a residue. The residue was purified by column chromatography (SiO₂, petroleum ether/ethyl acetate=1/0 to 10/1) to give methyl 4-(3-bromo-2-methyl-phenoxy)cyclohexanecarboxylate (49 g, 80.5 mmol, 50.2% yield, 53.8% purity) as a colorless oil.

### Step B. Procedure for Preparation of [4-(3-bromo-2-methyl-phenoxy) cyclohexyl]methanol

To a solution of methyl 4-(3-bromo-2-methyl-phenoxy)cyclohexanecarboxylate (49 g, 80.57 mmol, 53.8% purity, 1 equiv.) in THE (500 mL) was added LiAlH₄ (3.97 g, 104.73 mmol, 1.3 equiv.) dropwise at 0° C. under N₂ atmosphere. The reaction mixture was stirred under N₂ atmosphere at 0° C. for 2 hours. The reaction mixture was quenched with Na₂SO₄·10 H₂O (2 g) and slowly warmed to 20° C. The mixture was filtered and the filter cake was washed with DCM (500 mL). Then the organic layers were concentrated under reduced pressure to give a residue. The residue was purified by column chromatography (SiO₂, petroleum ether/ethyl acetate=20/1 to 5/1) to give [4-(3-bromo-2-methyl-phenoxy) cyclohexyl]methanol (15.7 g, 52.4 mmol, 65.1% yield) as a yellow oil.

¹H NMR (400 MHz, CDCl₃-d) δ=7.14 (d, J=8.0 Hz, 1H), 6.98 (t, J=8.0 Hz, 1H), 6.82 (d, J=8.4 Hz, 1H), 4.12-4.07 (m, 1H), 3.51 (d, J=6.4 Hz, 2H), 2.31 (s, 3H), 2.23-2.14 (m, 2H), 1.96-1.88 (m, 2H), 1.63-1.54 (m, 1H), 1.50-1.46 (m, 2H), 1.16-1.05 (m, 2H)

### Step C. Procedure for Preparation of 4-(3-bromo-2-methyl-phenoxy)cyclohexanecarbaldehyde

A solution of DMSO (16.40 g, 209.89 mmol, 16.40 mL, 4 equiv.) in DCM (10 mL) was added dropwise to a solution of oxalyl dichloride (13.32 g, 104.95 mmol, 9.19 mL, 2 equiv.) in DCM (130 mL) at −70° C. under N₂ atmosphere. The mixture was stirred at −70° C. for 30 minutes. After which time [4-(3-bromo-2-methyl-phenoxy)cyclohexyl]methanol (15.7 g, 52.47 mmol, 1 equiv.) in DCM (10 mL) was added dropwise at −70° C. The solution was stirred for 30 minutes at −70° C. Then TEA (31.86 g, 314.84 mmol, 43.82 mL, 6 equiv.) was added into the solution. The solution was stirred at 25° C. for 1 hour under N₂ atmosphere. The reaction mixture was diluted with water (20 mL) and extracted with DCM 60 mL (20 mL×3). The combined organic layers were washed with brine 20 mL (10 mL×2), dried over sodium sulfate, filtered, and concentrated under reduced pressure to give 4-(3-bromo-2-methyl-phenoxy)cyclohexanecarbaldehyde (22 g, crude) as a yellow oil.

### Step D. Procedure for Preparation of ethyl 3-[4-(3-bromo-2-methyl-phenoxy)cyclohexyl]-2, 2-difluoro-3-hydroxy-propanoate

To a solution of Zn (2.50 g, 38.28 mmol, 2.5 equiv.) in THE (5 mL) was added 1,2-dibromoethane (287.62 mg, 1.53 mmol, 115.51 μL, 0.1 equiv.) and ethyl 2-bromo-2,2-difluoro-acetate (4.35 g, 21.43 mmol, 2.75 mL, 1.4 equiv.) at 60° C. The reaction mixture was degassed and purged with N₂ three times, and then the mixture was stirred at 60° C. for 0.5 hours under N₂ atmosphere. Then a solution of 4-(3-bromo-2-methyl-phenoxy)cyclohexanecarbaldehyde (1.5 g, 5.05 mmol, 0.33 equiv.) in THF (1 mL) was added dropwise at 60° C. The mixture was stirred at 60° C. for 16 hours. The mixture was cooled to 20° C. and then filtered, the filtrate was quenched by iN HCl (100 mL), and then extracted with ethyl acetate 300 mL (100 mL×3). The combined organic layers were washed with brine saturated NaHCO₃ (300 mL) and brine 100 mL, dried over sodium sulfate, filtered, and concentrated under reduced pressure to give a residue. The residue was purified by flash silica gel chromatography (eluent of 0˜10% ethyl acetate/petroleum ether) to give ethyl 3-[4-(3-bromo-2-methyl-phenoxy)cyclohexyl]-2, 2-difluoro-3-hydroxy-propanoate (670 mg, 1.5 mmol, 10.3% yield) as a colorless oil.

¹H NMR (400 MHz, CDCl₃-d) δ=7.15 (d, J=8.0 Hz, 1H), 6.98 (t, J=8.0 Hz, 1H), 6.81 (d, J=8.0 Hz, 1H), 4.38 (q, J=7.2 Hz, 2H), 4.15-4.07 (m, 1H), 3.95-3.85 (m, 1H), 2.30 (s, 3H), 2.25-2.17 (m, 2H), 2.13-2.05 (m, 2H), 1.94-1.87 (m, 1H), 1.82-1.73 (m, 1H), 1.52-1.43 (m, 3H), 1.39 (t, J=7.2 Hz, 3H)

### Step E. Procedure for Preparation of ethyl 3-[4-(3-bromo-2-methyl-phenoxy)cyclohexyl]-2, 2-difluoro-3-methylsulfanylcarbothioyloxy-propanoate

To a solution of ethyl 3-[4-(3-bromo-2-methyl-phenoxy)cyclohexyl]-2,2-difluoro-3-hydroxy-propanoate (670 mg, 1.59 mmol, 1 equiv.) in DMF (8.5 mL) was added DBU (968.50 mg, 6.36 mmol, 958.91 μL, 4 equiv.) and CS₂ (1.21 g, 15.90 mmol, 961.07 μL, 10 equiv.) at 20° C. The mixture was stirred at 20° C. for 1 hour under N₂ atmosphere. Then Mel (2.26 g, 15.90 mmol, 990.11 μL, 10 equiv.) was added dropwise at 20° C. and the mixture was stirred at 20° C. for 1 hour under N₂ atmosphere. The mixture was diluted with water (10 mL) and extracted with ethyl acetate 60 mL (20 mL×3). The combined organic layers were washed with brine 20 mL (10 mL×2), dried over sodium sulfate, filtered, and concentrated under reduced pressure to give a residue. The residue was purified by flash silica gel chromatography (eluent of 0˜10% ethyl acetate/petroleum ether) to give ethyl 3-[4-(3-bromo-2-methyl-phenoxy)cyclohexyl]-2, 2-difluoro-3-methylsulfanylcarbothioyloxy-propanoate (764 mg, 1.4 mmol, 93.9% yield) as a colorless oil.

¹H NMR (400 MHz, CDCl₃-d) δ=7.15 (d, J=7.6 Hz, 1H), 6.97 (t, J=8.0 Hz, 1H), 6.79 (d, J=8.4 Hz, 1H), 6.31-6.22 (m, 1H), 4.30 (q, J=7.2 Hz, 2H), 4.15-4.04 (m, 1H), 2.63-2.56 (m, 3H), 2.29 (s, 3H), 2.23-2.17 (m, 2H), 2.16-2.08 (m, 1H), 2.04-1.95 (m, 2H), 1.52-1.37 (m, 4H), 1.34 (t, J=7.2 Hz, 3H)

### Step F. Procedure for Preparation of ethyl 3-[4-(3-bromo-2-methyl-phenoxy)cyclohexyl]-2, 2-difluoro-propanoate

A solution of ethyl 3-[4-(3-bromo-2-methyl-phenoxy)cyclohexyl]-2,2-difluoro-3-methylsulfanylcarbothioyloxy-propanoate (764 mg, 1.49 mmol, 1 equiv.), 2-tert-butylperoxy-2-methyl-propane (240.28 mg, 1.64 mmol, 302.62 μL, 1.1 equiv.) and phenylphosphonoylbenzene (664.48 mg, 3.29 mmol, 2.2 equiv.) in dioxane (8 mL) was degassed and purged with N₂ three times, and then the mixture was stirred at 100° C. for 24 hours under N₂ atmosphere. The reaction mixture was filtered and concentrated under reduced pressure to give a residue. The residue was purified by flash silica gel chromatography (eluent of 0˜10% ethyl acetate/petroleum ether) to give ethyl 3-[4-(3-bromo-2-methyl-phenoxy)cyclohexyl]-2,2-difluoro-propanoate (580 mg, 1.4 mmol, 95.8% yield) as a colorless oil.

¹H NMR (400 MHz, CDCl₃-d) δ=7.15 (d, J=7.6 Hz, 1H), 6.97 (t, J=8.0 Hz, 1H), 6.80 (d, J=8.0 Hz, 1H), 4.34 (q, J=7.2 Hz, 2H), 4.14-4.05 (m, 1H), 2.30 (s, 3H), 2.18-2.10 (m, 2H), 2.08-1.98 (m, 2H), 1.95 (d, J=12.8 Hz, 2H), 1.77-1.64 (m, 1H), 1.54-1.45 (m, 2H), 1.37 (t, J=7.2 Hz, 3H), 1.23-1.10 (m, 2H)

### Step G. Procedure for Preparation of tert-butyl 6-(8-(benzo[d]thiazol-2-ylcarbamoyl)-3,4-dihydroisoquinolin-2 (1H)-yl)-3-(3-(((1r,4r)-4-(3-ethoxy-2,2-difluoro-3-oxopropyl)cyclohexyl)oxy)-2-methylphenyl)picolinate

tert-Butyl 6-[8-(1, 3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (392.97 mg, 641.54 mol, 1.2 equiv.), ethyl 3-[4-(3-bromo-2-methyl-phenoxy)cyclohexyl]-2,2-difluoro-propanoate (200 mg, 493.49 mol, 1 equiv.), KF (86.02 mg, 1.48 mmol, 34.68 μL, 3 equiv.), and [2-(2-aminophenyl) phenyl]palladium(1+);bis(1-adamantyl)-butyl-phosphane;methanesulfonate (71.88 mg, 98.70 mol, 0.2 equiv.) were taken up into a microwave tube in dioxane (1 mL) and water (0.1 mL). The sealed tube was heated at 80° C. for 1 hour under microwave. The reaction mixture was filtered and concentrated under reduced pressure to give a residue. The residue was purified by prep-TLC (SiO₂, petroleum ether/ethyl acetate=1:1). Compound tert-butyl 6-(8-(benzo[d]thiazol-2-ylcarbamoyl)-3,4-dihydroisoquinolin-2 (1H)-yl)-3-(3-(((1r,4r)-4-(3-ethoxy-2,2-difluoro-3-oxopropyl)cyclohexyl)oxy)-2-methylphenyl)picolinate (370 mg, 221.2 mol, 44.8% yield) was obtained as a yellow oil.

MS (ESI) m/z: 811.6 [M+H]⁺.

### Step H. Procedure for Preparation of 3-((1r,4r)-4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)cyclohexyl)-2,2-difluoropropanoic acid

To a solution of tert-butyl 6-(8-(benzo[d]thiazol-2-ylcarbamoyl)-3,4-dihydroisoquinolin-2 (1H)-yl)-3-(3-(((1r,4r)-4-(3-ethoxy-2,2-difluoro-3-oxopropyl)cyclohexyl)oxy)-2-methylphenyl)picolinate (370 mg, 221.28 mol, 48.5% purity, 1 equiv.) in THE (2 mL) and water (0.2 mL) was added LiOH·H₂O (27.86 mg, 663.85 mol, 3 equiv.). The mixture was stirred at 25° C. for 16 hours. The reaction mixture was filtered and concentrated under reduced pressure to give a residue. The residue was purified by prep-HPLC. 3-((1r,4r)-4-(3-(6-(8-(Benzo[d]thiazol-2-ylcarbamoyl)-3,4-dihydroisoquinolin-2 (1H)-yl)-2-(tert-butoxycarbonyl)pyridin-3-yl)-2-methylphenoxy)cyclohexyl)-2,2-difluoropropanoic acid (35 mg, 43.5 mol, 19.6% yield) was obtained as a white oil.

MS (ESI) m/z: 783.5 [M+H]⁺.

### Step I. Procedure for Preparation of tert-butyl 6-(8-(benzo[d]thiazol-2-ylcarbamoyl)-3,4-dihydroisoquinolin-2 (1H)-yl)-3-(3-(((1S,4r)-4-(2,2-difluoro-3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropyl)cyclohexyl)oxy)-2-methylphenyl)picolinate

To a solution of 3-((1r,4r)-4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)cyclohexyl)-2,2-difluoropropanoic acid (30 mg, 38.32 mol, 1 equiv.) in DMF (1 mL) was added DIPEA (14.86 mg, 114.96 mol, 20.02 μL, 3 equiv.), HATU (21.86 mg, 57.48 mol, 1.5 equiv.), and (2S, 4R)-1-[(2S)-2-amino-3,3-dimethyl-butanoyl]-4-hydroxy-N-[(1S)-1-[4-(4-methylthiazol-5-yl) phenyl]ethyl]pyrrolidine-2-carboxamide (25.55 mg, 57.48 mol, 1.5 equiv.) at 25° C. The reaction mixture was stirred at 25° C. for 16 hours. The reaction mixture was diluted with water (5 mL). The mixture was triturated with water (5 mL) at 25° C. for 10 min. The mixture was then filtered to give the compound tert-butyl 6-(8-(benzo[d]thiazol-2-ylcarbamoyl)-3,4-dihydroisoquinolin-2 (1H)-yl)-3-(3-(((1S,4r)-4-(2,2-difluoro-3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropyl)cyclohexyl)oxy)-2-methylphenyl)picolinate (40 mg, 30.5 mol, 79.8% yield, 92.5% purity) as a yellow solid.

MS (ESI) m/z: 1209.5 [M+H]⁺.

### Step J. Procedure for Preparation of 6-(8-(benzo[d]thiazol-2-ylcarbamoyl)-3,4-dihydroisoquinolin-2 (1H)-yl)-3-(3-(((1S,4r)-4-(2,2-difluoro-3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropyl)cyclohexyl)oxy)-2-methylphenyl)picolinic acid

A solution of tert-butyl 6-(8-(benzo[d]thiazol-2-ylcarbamoyl)-3,4-dihydroisoquinolin-2 (1H)-yl)-3-(3-(((1S,4r)-4-(2,2-difluoro-3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropyl)cyclohexyl)oxy)-2-methylphenyl)picolinate (40 mg, 33.07 mol, 1 equiv.) in TFA (1 mL) and DCM (1 mL) was stirred at 20° C. for 16 hours. The reaction mixture was filtered and concentrated under reduced pressure to give a residue. The residue was purified by prep-HPLC. 6-(8-(Benzo[d]thiazol-2-ylcarbamoyl)-3,4-dihydroisoquinolin-2 (1H)-yl)-3-(3-(((1S,4r)-4-(2,2-difluoro-3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropyl)cyclohexyl)oxy)-2-methylphenyl)picolinic acid (17.8 mg, 14.6 mol, 44.3% yield) was obtained as a pink solid.

MS (ESI) m/z: 1153.4 [M+H]⁺.

¹H NMR (400 MHz, DMSO-d₆) δ=8.98 (s, 1H), 8.45 (d, J=7.6 Hz, 1H), 8.03 (d, J=7.6 Hz, 1H), 7.90 (d, J=8.8 Hz, 1H), 7.78 (d, J=8.0 Hz, 1H), 7.62 (d, J=7.6 Hz, 1H), 7.49-7.42 (m, 5H), 7.40-7.30 (m, 5H), 7.09-7.04 (m, 1H), 6.93 (dd, J=8.4, 16.0 Hz, 2H), 6.61 (d, J=7.2 Hz, 1H), 5.18-5.07 (m, 1H), 4.97 (s, 2H), 4.91 (t, J=7.2 Hz, 1H), 4.55 (d, J=9.2 Hz, 1H), 4.46 (t, J=8.0 Hz, 1H), 4.28 (s, 1H), 4.21-4.13 (m, 1H), 3.91 (t, J=5.6 Hz, 2H), 3.65-3.54 (m, 2H), 3.02 (t, J=5.6 Hz, 2H), 2.45 (s, 3H), 2.11-2.01 (m, 5H), 1.86 (s, 3H), 1.85-1.73 (m, 4H), 1.63-1.53 (m, 1H), 1.37 (d, J=7.2 Hz, 3H), 1.25-1.17 (m, 2H), 0.99-0.95 (m, 9H)

### Example 19. Preparation of Compound 118a

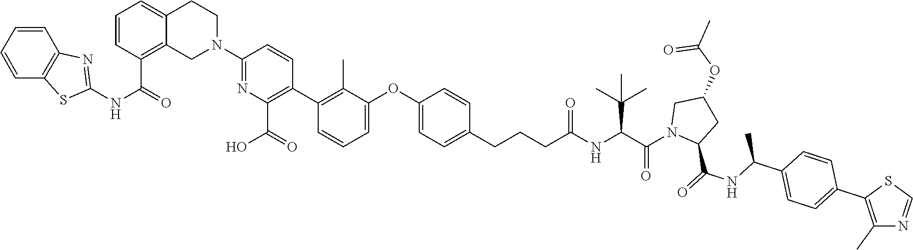 [SMILES: CC(=O)O[C@@H]1C[C@@H](C(=O)N[C@@H](C)c2ccc(-c3scnc3C)cc2)N(C(=O)[C@@H](NC(=O)CCCc2ccc(Oc3cccc(-c4ccc(N5CCc6cccc(C(=O)Nc7nc8ccccc8s7)c6C5)nc4C(=O)O)c3C)cc2)C(C)(C)C)C1]

### 3-[3-[4-[4-[[(1S)-1-[(2S,4R)-4-acetoxy-2-[[(1S)-1-[4-(4-methylthiazol-5-yl)phenyl]ethyl]carbamoyl]pyrrolidine-1-carbonyl]-2,2-dimethyl-propyl]amino]-4-oxo-butyl]phenoxy]-2-methyl-phenyl]-6-[8-(1,3-benzothiazol-2-ylcarbamoyl)-3,4-dihydro-1H-isoquinolin-2-yl]pyridine-2-carboxylic acid

### Step A. Procedure for Preparation of ethyl 4-(4-hydroxyphenyl) butanoate

A mixture of 4-(4-hydroxyphenyl)butanoic acid (600 mg, 3.33 mmol, 1.0 equiv.), SOCl₂ (792.25 mg, 6.66 mmol, 483.08 μL, 2.0 equiv.) in EtOH (10 mL) was degassed and purged with N₂ three times, and then the mixture was stirred at 90° C. for 16 hours under N₂ atmosphere. The reaction mixture was filtered and concentrated under reduced pressure to give a residue. Ethyl 4-(4-hydroxyphenyl) butanoate (1.5 g, crude) was obtained as a colorless oil, which was used in the next step without further purification.

MS (ESI) m/z: 207.1 [M+H]⁺.

### Step B. Procedure for Preparation of ethyl 4-[4-(3-bromo-2-methyl-phenoxy)phenyl]butanoate

A mixture of ethyl 4-(4-hydroxyphenyl)butanoate (700 mg, 3.36 mmol, 1.0 equiv.), 1,3-dibromo-2-methyl-benzene (1.68 g, 6.72 mmol, 2.0 equiv.), Cs₂CO₃ (1.31 g, 4.03 mmol, 1.2 equiv.), CuI (320.08 mg, 1.68 mmol, 0.5 equiv.), and 2,2,6,6-tetramethylheptane-3,5-dione (154.85 mg, 840.32 μmol, 173.02 μL, 0.25 equiv.) in NMP (12 mL) was degassed and purged with N₂ three times, and then the mixture was stirred at 120° C. for 12 hours under N₂ atmosphere. The residue was diluted with ethyl acetate (80 mL) and extracted with water 30 mL (15 mL×2), dried over sodium sulfate, filtered, and concentrated under reduced pressure to give a residue. The residue was purified by flash silica gel chromatography (eluent of 0˜5% ethyl acetate/petroleum ether). Ethyl 4-[4-(3-bromo-2-methyl-phenoxy)phenyl]butanoate (1.5 g, 3.9 mmol, 59.1% yield) was obtained as a yellow oil.

### Step C. Procedure for Preparation of tert-butyl 6-[8-(1, 3-benzothiazol-2-ylcarbamoyl)-3, 4-dihydro-1H-isoquinolin-2-yl]-3-[3-[4-(4-ethoxy-4-oxo-butyl)phenoxy]-2-methyl-phenyl]pyridine-2-carboxylate

To a solution of ethyl 4-[4-(3-bromo-2-methyl-phenoxy)phenyl]butanoate (500 mg, 1.33 mmol, 1.0 equiv.) and tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (974.17 mg, 1.59 mmol, 1.2 equiv.) in dioxane (5 mL) and water (1 mL) was added Ad₂nBuP Pd G₃ (cataCXium® A Pd G₃) (193.04 mg, 265.06 μmol, 0.2 equiv.) and K₂CO₃ (274.75 mg, 1.99 mmol, 1.5 equiv.). The mixture was stirred at 100° C. for 1 hour under microwave. The reaction mixture was filtered and concentrated under reduced pressure to remove solvent. The residue was diluted with ethyl acetate (80 mL) and extracted with water 30 mL (15 mL×2), dried over sodium sulfate, filtered, and concentrated under reduced pressure to give a residue. The residue was purified by flash silica gel chromatography (eluent of 0˜40% ethyl acetate/petroleum ether). tert-Butyl 6-[8-(1, 3-benzothiazol-2-ylcarbamoyl)-3, 4-dihydro-1H-isoquinolin-2-yl]-3-[3-[4-(4-ethoxy-4-oxo-butyl)phenoxy]-2-methyl-phenyl]pyridine-2-carboxylate (1.9 g, crude) was obtained as a brown oil.

¹H NMR (400 MHz, DMSO-d₆) δ=7.42 (d, J=8.0 Hz, 1H), 7.23-7.11 (m, 3H), 6.91-6.81 (m, 3H), 4.09-3.98 (m, 2H), 3.59 (s, 1H), 2.57 (t, J=7.6 Hz, 2H), 2.27 (s, 3H), 1.99 (s, 1H), 1.81 (m, J=7.6 Hz, 2H), 1.18 (t, J=7.2 Hz, 3H)

### Step D. Procedure for Preparation of 4-[4-[3-[6-[8-(1, 3-benzothiazol-2-ylcarbamoyl)-3, 4-dihydro-1H-isoquinolin-2-yl]-2-tert-butoxycarbonyl-3-pyridyl]-2-methyl-phenoxy]phenyl]butanoic acid

To a solution of tert-butyl 6-[8-(1, 3-benzothiazol-2-ylcarbamoyl)-3,4-dihydro-1H-isoquinolin-2-yl]-3-[3-[4-(4-ethoxy-4-oxo-butyl)phenoxy]-2-methyl-phenyl]pyridine-2-carboxylate (950 mg, 1.21 mmol, 1.0 equiv.) in MeOH (6 mL) and water (6 mL) was added LiOH·H₂O (152.75 mg, 3.64 mmol, 3 equiv.). The mixture was stirred at 25° C. for 12 hours and then diluted with water (20 mL). 1M HCl was used to adjust the mixture to pH=3-4. The precipitated solids are filtered and concentrated under reduced pressure to give a brown solid. 4-[4-[3-[6-[8-(1,3-Benzothiazol-2-ylcarbamoyl)-3,4-dihydro-1H-isoquinolin-2-yl]-2-tert-butoxycarbonyl-3-pyridyl]-2-methyl-phenoxy]phenyl]butanoic acid (1.5 g, crude) was obtained as a brown solid, which was used in the next step without further purification.

MS (ESI) m/z: 755.4 [M+H]⁺.

### Step E. Procedure for Preparation of tert-butyl 3-[3-[4-[4-[[(1S)-1-[(2S,4R)-4-acetoxy-2-[[(1S)-1-[4-(4-methylthiazol-5-yl)phenyl]ethyl]carbamoyl]pyrrolidine-1-carbonyl]-2,2-dimethyl-propyl]amino]-4-oxo-butyl]phenoxy]-2-methyl-phenyl]-6-[8-(1,3-benzothiazol-2-ylcarbamoyl)-3,4-dihydro-1H-isoquinolin-2-yl]pyridine-2-carboxylate

To a solution of 4-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]phenyl]butanoic acid (150 mg, 198.70 μmol, 1.0 equiv.) and [(3R,5S)-1-[(2S)-2-amino-3,3-dimethyl-butanoyl]-5-[[(1S)-1-[4-(4-methylthiazol-5-yl)phenyl]ethyl]carbamoyl]pyrrolidin-3-yl]acetate (193.39 mg, 397.41 μmol, 1.5 equiv.) in DMF (2 mL) was added HATU (113.33 mg, 298.06 μmol, 1.2 equiv.) and DIEA (128.41 mg, 993.52 μmol, 173.05 μL, 1.5 equiv.). The mixture was stirred at 25° C. for 16 hours. The reaction mixture was filtered and concentrated under reduced pressure to give a residue. The residue was purified by column chromatography by prep-TLC (SiO₂, DCM:MeOH=15:1). tert-Butyl 3-[3-[4-[4-[[(1S)-1-[(2S,4R)-4-acetoxy-2-[[(1S)-1-[4-(4-methylthiazol-5-yl)phenyl]ethyl]carbamoyl]pyrrolidine-1-carbonyl]-2,2-dimethyl-propyl]amino]-4-oxo-butyl]phenoxy]-2-methyl-phenyl]-6-[8-(1,3-benzothiazol-2-ylcarbamoyl)-3,4-dihydro-1H-isoquinolin-2-yl]pyridine-2-carboxylate (50 mg, 40.8 μmol, 20.5% yield) was obtained as a yellow solid.

MS (ESI) m/z: 612.7 [M12+H]⁺.

### Step F. Procedure for Preparation of 3-[3-[4-[4-[[(1S)-1-[(2S,4R)-4-acetoxy-2-[[(1S)-1-[4-(4-methylthiazol-5-yl)phenyl]ethyl]carbamoyl]pyrrolidine-1-carbonyl]-2,2-dimethyl-propyl]amino]-4-oxo-butyl]phenoxy]-2-methyl-phenyl]-6-[8-(1,3-benzothiazol-2-ylcarbamoyl)-3,4-dihydro-1H-isoquinolin-2-yl]pyridine-2-carboxylic acid

To a solution of tert-butyl 3-[3-[4-[4-[[(1S)-1-[(2S,4R)-4-acetoxy-2-[[(1S)-1-[4-(4-methylthiazol-5-yl)phenyl]ethyl]carbamoyl]pyrrolidine-1-carbonyl]-2,2-dimethyl-propyl]amino]-4-oxo-butyl]phenoxy]-2-methyl-phenyl]-6-[8-(1,3-benzothiazol-2-ylcarbamoyl)-3,4-dihydro-1H-isoquinolin-2-yl]pyridine-2-carboxylate (50 mg, 40.87 μmol, 1 equiv.) in TFA (0.5 mL) and DCM (0.5 mL). The mixture was stirred at 25° C. for 2 hours. The reaction mixture was concentrated under reduced pressure to give a residue. The residue was purified by prep-HPLC. 3-[3-[4-[4-[[(1S)-1-[(2S,4R)-4-Acetoxy-2-[[(1S)-1-[4-(4-methylthiazol-5-yl)phenyl]ethyl]carbamoyl]pyrrolidine-1-carbonyl]-2,2-dimethyl-propyl]amino]-4-oxo-butyl]phenoxy]-2-methyl-phenyl]-6-[8-(1,3-benzothiazol-2-ylcarbamoyl)-3,4-dihydro-1H-isoquinolin-2-yl]pyridine-2-carboxylic acid (17.6 mg, 14.7 μmol, 35.9% yield, 97.4% purity) was obtained as a yellow solid.

MS (ESI) m/z: 1168.6 [M+H]⁺.

¹H NMR (400 MHz, DMSO-d₆) δ=12.86 (s, 1H), 12.75-12.43 (m, 1H), 9.00-8.96 (m, 1H), 8.39 (d, J=8.4 Hz, 1H), 8.03 (d, J=8.0 Hz, 1H), 7.86 (d, J=8.4 Hz, 1H), 7.79 (d, J=8.0 Hz, 1H), 7.62 (d, J=7.2 Hz, 1H), 7.53 (d, J=8.8 Hz, 1H), 7.50-7.41 (m, 4H), 7.40-7.33 (m, 4H), 7.20-7.10 (m, 3H), 7.01 (d, J=8.8 Hz, 1H), 6.90-6.78 (m, 4H), 5.18 (s, 1H), 5.00 (s, 2H), 4.94-4.86 (m, 1H), 4.46 (t, J=8.4 Hz, 1H), 4.36 (d, J=8.8 Hz, 1H), 4.01-3.89 (m, 3H), 3.79-3.65 (m, 1H), 3.03 (t, J=5.6 Hz, 2H), 2.47-2.44 (m, 3H), 2.27-2.15 (m, 4H), 2.03-1.93 (m, 5H), 1.90 (s, 3H), 1.80-1.67 (m, 2H), 1.37 (d, J=7.2 Hz, 3H), 0.98-0.93 (m, 9H).

### Example 20. Preparation of Compound 119a

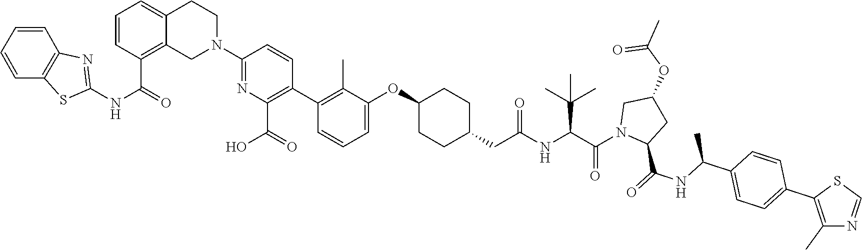 [SMILES: CC(=O)O[C@@H]1C[C@@H](C(=O)N[C@@H](C)c2ccc(-c3scnc3C)cc2)N(C(=O)[C@@H](NC(=O)C[C@H]2CC[C@H](Oc3cccc(-c4ccc(N5CCc6cccc(C(=O)Nc7nc8ccccc8s7)c6C5)nc4C(=O)O)c3C)CC2)C(C)(C)C)C1]

### 3-(3-(((1S,4r)-4-(2-(((S)-1-((2S,4R)-4-acetoxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)oxy)-2-methylphenyl)-6-(8-(benzo[d]thiazol-2-ylcarbamoyl)-3,4-dihydroisoquinolin-2 (1H)-yl)picolinic acid

### Step A. Procedure for Preparation of methyl 2-[4-(3-bromo-2-methyl-phenoxy)cyclohexyl]acetate

To a solution of methyl 2-(4-hydroxycyclohexyl)acetate (2 g, 11.61 mmol, 1 equiv.) in toluene (20 mL) was added 2-(tributyl-5-phosphanylidene)acetonitrile (3.64 g, 15.10 mmol, 1.5 equiv.) and 3-bromo-2-methyl-phenol (2.39 g, 12.77 mmol, 1.1 equiv.). The mixture was stirred at 120° C. for 12 hours. The reaction mixture was concentrated under reduced pressure to give a residue. The residue was purified by prep-HPLC to give methyl 2-[4-(3-bromo-2-methyl-phenoxy)cyclohexyl]acetate (3.8 g, 11.1 mmol, 95.8% yield) as a yellow oil.

### Step B. Procedure for Preparation of methyl 2-((1r,4r)-4-(3-bromo-2-methylphenoxy)cyclohexyl)acetate

The methyl 2-[4-(3-bromo-2-methyl-phenoxy)cyclohexyl]acetate residue was purified by prep-HPLC to give methyl 2-((1r,4r)-4-(3-bromo-2-methylphenoxy)cyclohexyl)acetate (320 mg, 937.7 μmol, 26.6% yield) as a yellow oil.

¹H NMR (400 MHz, CDCl₃) δ=7.16 (d, J=8.0 Hz, 1H), 7.02-6.94 (m, 1H), 6.84-6.78 (m, 1H), 4.15-4.05 (m, 1H), 3.70 (s, 3H), 2.32 (s, 3H), 2.26 (d, J=6.8 Hz, 2H), 2.19-2.12 (m, 2H), 1.91-1.87 (m, 2H), 1.59-1.53 (m, 1H), 1.55-1.46 (m, 2H), 1.19-1.08 (m, 2H)

### Step C. Procedure for Preparation of tert-butyl 6-(8-(benzo[d]thiazol-2-ylcarbamoyl)-3,4-dihydroisoquinolin-2 (1H)-yl)-3-(3-(((1r,4r)-4-(2-methoxy-2-oxoethyl)cyclohexyl)oxy)-2-methylphenyl)picolinate

A mixture of tert-butyl 6-(8-(benzo[d]thiazol-2-ylcarbamoyl)-3,4-dihydroisoquinolin-2 (1H)-yl)-3-(4,4,5,5-tetramethyl-1,3,2-dioxaborolan-2-yl)picolinate (180 mg, 293.86 μmol, 1 equiv.), methyl 2-((1r,4r)-4-(3-bromo-2-methylphenoxy)cyclohexyl)acetate (100.28 mg, 293.86 μmol, 1 equiv.), [2-(2-aminophenyl)phenyl]palladium(1+);bis(1-adamantyl)-butyl-phosphane;methanesulfonate (21.40 mg, 29.39 μmol, 0.1 equiv.), and KF (51.22 mg, 881.57 μmol, 20.65 μL, 3 equiv.) in dioxane (4 mL) and water (1 mL) was degassed and purged with N₂ three times and then the mixture was stirred at 100° C. for 1 hour. The reaction mixture was filtered and concentrated under reduced pressure to give a residue. The residue was purified by column chromatography (SiO₂, petroleum ether/ethyl acetate=20/1 to 1/1) to give tert-butyl 6-(8-(benzo[d]thiazol-2-ylcarbamoyl)-3,4-dihydroisoquinolin-2 (1H)-yl)-3-(3-(((1r,4r)-4-(2-methoxy-2-oxoethyl)cyclohexyl)oxy)-2-methylphenyl)picolinate (150 mg, 200.8 μmol, 68.3% yield) as a yellow solid.

MS (ESI) m/z: 747.3 [M+H]⁺.

¹H NMR (400 MHz, CDCl₃) δ=7.77 (d, J=7.6 Hz, 1H), 7.52 (d, J=7.6 Hz, 1H), 7.41 (d, J=7.6 Hz, 1H), 7.31-7.24 (m, 4H), 7.19-7.15 (m, 1H), 7.02-6.96 (m, 1H), 6.81 (d, J=8.8 Hz, 1H), 6.76 (d, J=8.4 Hz, 1H), 6.60 (d, J=7.6 Hz, 1H), 5.03-4.88 (m, 2H), 4.08-4.00 (m, 3H), 3.61 (s, 3H), 2.98 (t, J=5.6 Hz, 2H), 2.18 (d, J=6.8 Hz, 2H), 2.12-2.06 (m, 2H), 1.89 (s, 3H), 1.80 (d, J=8.4 Hz, 4H), 1.48-1.32 (m, 3H), 1.06 (s, 9H)

### Step D. Procedure for Preparation of 2-((1r,4r)-4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)cyclohexyl)acetic acid

To a solution of tert-butyl 6-(8-(benzo[d]thiazol-2-ylcarbamoyl)-3,4-dihydroisoquinolin-2 (1H)-yl)-3-(3-(((1r,4r)-4-(2-methoxy-2-oxoethyl)cyclohexyl)oxy)-2-methylphenyl)picolinate (150 mg, 200.83 μmol, 1 equiv.) in THE (2 mL) and water (0.5 mL) was added LiOH·H₂O (25.28 mg, 602.48 μmol, 3 equiv.). The mixture was stirred at 25° C. for 12 hours. The reaction mixture was acidified to pH=5 with 1M HCl and extracted with ethyl acetate 30 mL (30 mL×3). The combined organic layers were washed with brine 30 mL, dried over sodium sulfate, filtered, and concentrated under reduced pressure to give 2-((1r,4r)-4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)cyclohexyl)acetic acid (140 mg, 191.0 μmol, 95.2% yield) as a yellow solid.

MS (ESI) m/z: 733.5 [M+H]⁺.

### Step E. Procedure for Preparation of tert-butyl 3-(3-(((1S,4r)-4-(2-(((S)-1-((2S,4R)-4-acetoxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)oxy)-2-methylphenyl)-6-(8-(benzo[d]thiazol-2-ylcarbamoyl)-3,4-dihydroisoquinolin-2 (1H)-yl)picolinate

To a solution of 2-((1r,4r)-4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)cyclohexyl)acetic acid (100 mg, 136.45 μmol, 1 equiv.) in DMF (1 mL) was added DIEA (52.90 mg, 409.34 μmol, 71.30 μL, 3 equiv.), HATU (62.26 mg, 163.74 μmol, 1.2 equiv.), and [(3R,5S)-1-[(2S)-2-amino-3,3-dimethyl-butanoyl]-5-[[(1S)-1-[4-(4-methylthiazol-5-yl)phenyl]ethyl]carbamoyl]pyrrolidin-3-yl]acetate (79.09 mg, 162.53 μmol, 1.0 equiv.). The mixture was stirred at 25° C. for 1 hour. The reaction was diluted with water (20 mL) and extracted with DCM 40 mL (20 mL×2). The combined organic layers were washed with brine (30 mL), dried over sodium sulfate, filtered, and concentrated under reduced pressure to give as a residue. The residue was purified by column chromatography (SiO₂, petroleum ether/ethyl acetate=5/1 to 0/1) to give tert-butyl 3-(3-(((1S,4r)-4-(2-(((S)-1-((2S,4R)-4-acetoxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)oxy)-2-methylphenyl)-6-(8-(benzo[d]thiazol-2-ylcarbamoyl)-3,4-dihydroisoquinolin-2 (1H)-yl)picolinate (50 mg, 41.6 μmol, 30.5% yield) as a yellow oil.

MS (ESI) m/z: 1202.4 [M+H]⁺.

### Step F. Procedure for Preparation of 3-(3-(((1S,4r)-4-(2-(((S)-1-((2S,4R)-4-acetoxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)oxy)-2-methylphenyl)-6-(8-(benzo[d]thiazol-2-ylcarbamoyl)-3,4-dihydroisoquinolin-2 (1H)-yl)picolinic acid

To a solution of tert-butyl 3-(3-(((1S,4r)-4-(2-(((S)-1-((2S,4R)-4-acetoxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)oxy)-2-methylphenyl)-6-(8-(benzo[d]thiazol-2-ylcarbamoyl)-3,4-dihydroisoquinolin-2 (1H)-yl)picolinate (50 mg, 41.61 μmol, 1 equiv.) in DCM (1 mL) was added TFA (1.54 g, 13.51 mmol, 1 mL, 324.56 equiv.). The mixture was stirred at 40° C. for 1 hour. The reaction mixture was concentrated under reduced pressure to give a residue. The residue was purified by prep-HPLC to give 3-(3-(((1S,4r)-4-(2-(((S)-1-((2S,4R)-4-acetoxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)oxy)-2-methylphenyl)-6-(8-(benzo[d]thiazol-2-ylcarbamoyl)-3,4-dihydroisoquinolin-2 (1H)-yl)picolinic acid (11.9 mg, 10.0 μmol, 24.2% yield) as a white solid.

MS (ESI) m/z: 1145.4 [M+H]⁺.

¹H NMR (400 MHz, DMSO-d₆) δ=12.79 (s, 1H), 12.64-12.38 (m, 1H), 8.91 (s, 1H), 8.37-8.31 (m, 1H), 7.96 (d, J=8.0 Hz, 1H), 7.80-7.69 (m, 2H), 7.55 (d, J=7.2 Hz, 1H), 7.40-7.34 (m, 5H), 7.32-7.27 (m, 4H), 7.03-6.97 (m, 1H), 6.91-6.83 (m, 2H), 6.54 (d, J=7.2 Hz, 1H), 5.13-5.08 (m, 1H), 4.90 (s, 2H), 4.84 (t, J=7.2 Hz, 1H), 4.41-4.35 (m, 1H), 4.29 (d, J=8.8 Hz, 1H), 4.14-4.08 (m, 1H), 3.94-3.90 (m, 1H), 3.84 (t, J=5.2 Hz, 2H), 3.67-3.62 (m, 2H), 2.96 (d, J=5.6 Hz, 2H), 2.38 (s, 3H), 2.17-2.08 (m, 2H), 2.04-1.95 (m, 4H), 1.92 (s, 3H), 1.79 (s, 3H), 1.68-1.60 (m, 3H), 1.30 (d, J=6.8 Hz, 4H), 1.06-0.99 (m, 2H), 0.88 (s, 9H)

### Example 21. Preparation of Compound 120a

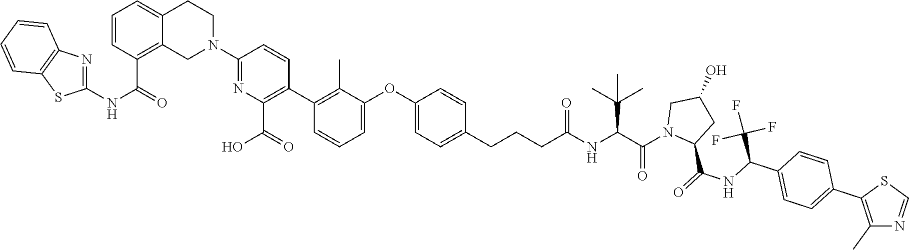 [SMILES: Cc1ncsc1-c1ccc([C@@H](NC(=O)[C@@H]2C[C@@H](O)CN2C(=O)[C@@H](NC(=O)CCCc2ccc(Oc3cccc(-c4ccc(N5CCc6cccc(C(=O)Nc7nc8ccccc8s7)c6C5)nc4C(=O)O)c3C)cc2)C(C)(C)C)C(F)(F)F)cc1]

### 6-[8-(1,3-benzothiazol-2-ylcarbamoyl)-3,4-dihydro-1H-isoquinolin-2-yl]-3-[3-[4-[4-[[(1S)-1-[(2S,4R)-4-hydroxy-2-[[(1R)-2,2,2-trifluoro-1-[4-(4-methylthiazol-5-yl)phenyl]ethyl]carbamoyl]pyrrolidine-1-carbonyl]-2,2-dimethyl-propyl]amino]-4-oxo-butyl]phenoxy]-2-methyl-phenyl]pyridine-2-carboxylic acid

### Step A. Procedure for Preparation of methyl (2S,4R)-1-[(2S)-2-(tert-butoxycarbonylamino)-3,3-dimethyl-butanoyl]-4-hydroxy-pyrrolidine-2-carboxylate

A mixture of (2S)-2-(tert-butoxycarbonylamino)-3,3-dimethyl-butanoic acid (2 g, 8.65 mmol, 1 equiv.), methyl (2S,4R)-4-hydroxypyrrolidine-2-carboxylate (1.26 g, 8.65 mmol, 1 equiv.), HATU (3.62 g, 9.51 mmol, 1.1 equiv.), and DIEA (3.35 g, 25.94 mmol, 4.52 mL, 3 equiv.) in DMF (20 mL) was degassed and purged with N₂ three times, and then the mixture was stirred at 25° C. for 12 hours under N₂ atmosphere. The reaction mixture was diluted with water (20 mL) and extracted with ethyl acetate 45 mL (15 mL×3). The combined organic layers were washed with brine 45 mL (15 mL×3), dried over sodium sulfate, filtered, and concentrated under reduced pressure to give a residue. The residue was purified by flash silica gel chromatography (eluent of 9% methanol/dichloromethane) to give methyl (2S,4R)-1-[(2S)-2-(tert-butoxycarbonylamino)-3,3-dimethyl-butanoyl]-4-hydroxy-pyrrolidine-2-carboxylate (1.3 g, 3.0 mmol, 35.3% yield, 84.1% purity) as a yellow oil.

MS (ESI) m/z: 381.1 [M+Na]⁺.

¹H NMR (400 MHz, DMSO-d₆) δ=6.53 (d, J=9.2 Hz, 1H), 5.76 (s, 1H), 5.22 (d, J=3.6 Hz, 1H), 4.38-4.29 (m, 2H), 3.60 (s, 3H), 1.40-1.35 (m, 9H), 1.25 (d, J=6.4 Hz, 4H), 0.96-0.88 (m, 9H).

### Step B. Procedure for Preparation of (2S,4R)-1-[(2S)-2-(tert-butoxycarbonylamino)-3,3-dimethyl-butanoyl]-4-hydroxy-pyrrolidine-2-carboxylic acid

To a solution of methyl (2S,4R)-1-[(2S)-2-(tert-butoxycarbonylamino)-3,3-dimethyl-butanoyl]-4-hydroxy-pyrrolidine-2-carboxylate (1.3 g, 3.63 mmol, 1.0 equiv.) in THE (15 mL) and water (5 mL) was added LiOH·H₂O (456.60 mg, 10.88 mmol, 3.0 equiv.). The mixture was stirred at 25° C. for 12 hours. The reaction mixture was adjusted pH to 3-4 and then extracted with ethyl acetate 15 mL (5 mL×3). The combined organic layers were dried over sodium sulfate, filtered, and concentrated under reduced pressure to give (2S,4R)-1-[(2S)-2-(tert-butoxycarbonylamino)-3,3-dimethyl-butanoyl]-4-hydroxy-pyrrolidine-2-carboxylic acid (700 mg, 2.0 mmol, 56.0% yield) as a yellow solid.

MS (ESI) m/z: 289.1 [M−56+H]⁺.

¹H NMR (400 MHz, DMSO-d₆) δ=12.84-11.86 (m, 1H), 6.47 (d, J=9.2 Hz, 1H), 5.75 (s, 1H), 5.18 (d, J=3.2 Hz, 1H), 4.32 (s, 1H), 4.26 (t, J=8.4 Hz, 1H), 4.19-4.10 (m, 1H), 3.68-3.55 (m, 2H), 2.15-2.05 (m, 1H), 1.95-1.80 (m, 1H), 1.38 (s, 9H), 0.93 (s, 8H).

### Step C. Procedure for Preparation of tert-butyl N-[(1S)-1-[(2S,4R)-2-[[(1R)-1-(4-bromophenyl)-2,2,2-trifluoro-ethyl]carbamoyl]-4-hydroxy-pyrrolidine-1-carbonyl]-2,2-dimethyl-propyl]carbamate

A mixture of (2S,4R)-1-[(2S)-2-(tert-butoxycarbonylamino)-3,3-dimethyl-butanoyl]-4-hydroxy-pyrrolidine-2-carboxylic acid (700 mg, 2.03 mmol, 1.0 equiv.), (1R)-1-(4-bromophenyl)-2,2,2-trifluoro-ethanamine (516.35 mg, 2.03 mmol, 1.0 equiv.), HATU (850.10 mg, 2.24 mmol, 1.1 equiv.), and DIEA (788.06 mg, 6.10 mmol, 1.06 mL, 3.0 equiv.) in DMF (8 mL) was stirred at 25° C. for 2 hours under N₂ atmosphere. The reaction mixture was diluted with water (5 mL) and extracted with ethyl acetate 15 mL (5 mL×3). The combined organic layers were washed with brine 15 mL (5 mL×3), dried over sodium sulfate, filtered, and concentrated under reduced pressure to give a residue. The residue was purified by flash silica gel chromatography (eluent of 5% methanol/dichloromethane) to give tert-butyl N-[(1S)-1-[(2S,4R)-2-[[(1R)-1-(4-bromophenyl)-2,2,2-trifluoro-ethyl]carbamoyl]-4-hydroxy-pyrrolidine-1-carbonyl]-2,2-dimethyl-propyl]carbamate (925 mg, 1.0 mmol, 53.5% yield, 68.2% purity) as a yellow oil.

MS (ESI) m/z: 602.2 [M+23]⁺.

¹H NMR (400 MHz, DMSO-d₆) δ=9.26 (d, J=9.2 Hz, 1H), 7.63 (d, J=8.4 Hz, 2H), 7.50 (d, J=8.4 Hz, 2H), 6.46 (d, J=9.2 Hz, 1H), 5.80 (t, J=8.8 Hz, 1H), 5.15 (d, J=2.8 Hz, 1H), 4.59-4.52 (m, 1H), 4.27 (s, 1H), 4.13 (d, J=9.2 Hz, 1H), 3.65-3.52 (m, 2H), 2.06-1.95 (m, 1H), 1.68 (d, J=4.8, 8.0, 12.4 Hz, 1H), 1.38 (s, 9H), 0.92 (s, 9H).

### Step D. Procedure for Preparation of tert-butyl N-[(1S)-1-[(2S,4R)-4-hydroxy-2-[[(1R)-2,2,2-trifluoro-1-[4-(4-methylthiazol-5-yl)phenyl]ethyl]carbamoyl]pyrrolidine-1-carbonyl]-2,2-dimethyl-propyl]carbamate

A mixture of tert-butyl N-[(1S)-1-[(2S,4R)-2-[[(1R)-1-(4-bromophenyl)-2,2,2-trifluoro-ethyl]carbamoyl]-4-hydroxy-pyrrolidine-1-carbonyl]-2,2-dimethyl-propyl]carbamate (925 mg, 1.59 mmol, 1.0 equiv.), 4-methylthiazole (316.03 mg, 3.19 mmol, 289.94 μL, 2.0 equiv.), Pd(OAc)₂ (35.78 mg, 159.36 μmol, 0.1 equiv.), and KOAc (312.81 mg, 3.19 mmol, 2.0 equiv.) in DMA (10 mL) was degassed and purged with N₂ three times, and then the mixture was stirred at 130° C. for 3 hours under N₂ atmosphere. The reaction mixture was diluted with water (6 mL) and extracted with DCM 9 mL (3 mL×3). The combined organic layers were washed with brine (5 mL×3), dried over sodium sulfate, filtered, and concentrated under reduced pressure to give a residue. The residue was purified by flash silica gel chromatography (eluent of 10% methanol/dichloromethane) to give tert-butyl N-[(1S)-1-[(2S,4R)-4-hydroxy-2-[[(1R)-2,2,2-trifluoro-1-[4-(4-methylthiazol-5-yl)phenyl]ethyl]carbamoyl]pyrrolidine-1-carbonyl]-2,2-dimethyl-propyl]carbamate (240 mg) as a brown oil.

MS (ESI) m/z: 621.2 [M+Na]⁺.

¹H NMR (400 MHz, CDCl₃) δ=9.02 (s, 1H), 7.67-7.61 (m, 2H), 7.58-7.52 (m, 2H), 5.75 (s, 2H), 5.14 (d, J=3.6 Hz, 1H), 4.60 (t, J=8.0 Hz, 1H), 4.29 (s, 1H), 2.94 (s, 2H), 2.78 (s, 2H), 2.47 (s, 3H), 1.96 (s, 2H), 1.39 (s, 9H), 0.93 (s, 9H).

### Step E. Procedure for Preparation of (2S,4R)-1-[(2S)-2-amino-3,3-dimethyl-butanoyl]-4-hydroxy-N-[(1R)-2,2,2-trifluoro-1-[4-(4-methylthiazol-5-yl)phenyl]ethyl]pyrrolidine-2-carboxamide

A solution of tert-butyl N-[(1S)-1-[(2S,4R)-4-hydroxy-2-[[(1R)-2,2,2-trifluoro-1-[4-(4-methylthiazol-5-yl)phenyl]ethyl]carbamoyl]pyrrolidine-1-carbonyl]-2,2-dimethyl-propyl]carbamate (240 mg, 400.88 μmol, 1.0 equiv.) in HCl/dioxane (1 mL) and DCM (3 mL) was stirred at 25° C. for 12 hours under N₂ atmosphere. The reaction mixture was concentrated under reduced pressure to give (2S,4R)-1-[(2S)-2-amino-3,3-dimethyl-butanoyl]-4-hydroxy-N-[(1R)-2,2,2-trifluoro-1-[4-(4-methylthiazol-5-yl)phenyl]ethyl]pyrrolidine-2-carboxamide (200 mg, crude, HCl) as a yellow solid.

MS (ESI) m/z: 499.2 [M+H]⁺.

### Step F. Procedure for Preparation of tert-butyl 6-[8-(1,3-benzothiazol-2-ylcarbamoyl)-3,4-dihydro-1H-isoquinolin-2-yl]-3-[3-[4-[4-[[(1S)-1-[(2S,4R)-4-hydroxy-2-[[(1R)-2,2,2-trifluoro-1-[4-(4-methylthiazol-5-yl)phenyl]ethyl]carbamoyl]pyrrolidine-1-carbonyl]-2,2-dimethyl-propyl]amino]-4-oxo-butyl]phenoxy]-2-methyl-phenyl]pyridine-2-carboxylate

A mixture of 4-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]phenyl]butanoic acid (58.79 mg, 77.88 μmol, 1.0 equiv.), (2S,4R)-1-[(2S)-2-amino-3,3-dimethyl-butanoyl]-4-hydroxy-N-[(1R)-2,2,2-trifluoro-1-[4-(4-methylthiazol-5-yl)phenyl]ethyl]pyrrolidine-2-carboxamide (50 mg, 93.45 μmol, 1.2 equiv., HCl), HATU (35.53 mg, 93.45 μmol, 1.2 equiv.), and DIEA (30.20 mg, 233.64 μmol, 40.69 μL, 3.0 equiv.) in DMF (2 mL) was stirred at 25° C. for 12 hours. The reaction mixture was diluted with water (5 mL) and extracted with ethyl acetate 9 mL (3 mL×3). The combined organic layers were washed with brine 9 mL (3 mL×3), dried over sodium sulfate, filtered, and concentrated under reduced pressure to give a residue. The residue was purified by prep-TLC (SiO₂, DCM:MeOH=10:1) to give tert-butyl 6-[8-(1,3-benzothiazol-2-ylcarbamoyl)-3,4-dihydro-1H-isoquinolin-2-yl]-3-[3-[4-[4-[[(1S)-1-[(2S,4R)-4-hydroxy-2-[[(1R)-2,2,2-trifluoro-1-[4-(4-methylthiazol-5-yl)phenyl]ethyl]carbamoyl]pyrrolidine-1-carbonyl]-2,2-dimethyl-propyl]amino]-4-oxo-butyl]phenoxy]-2-methyl-phenyl]pyridine-2-carboxylate (40 mg, crude) as a yellow oil.

MS (ESI) m/z: 618.6 [M12+H]⁺.

### Step G. Procedure for Preparation of 6-[8-(1,3-benzothiazol-2-ylcarbamoyl)-3,4-dihydro-1H-isoquinolin-2-yl]-3-[3-[4-[4-[[(1S)-1-[(2S,4R)-4-hydroxy-2-[[(1R)-2,2,2-trifluoro-1-[4-(4-methylthiazol-5-yl)phenyl]ethyl]carbamoyl]pyrrolidine-1-carbonyl]-2,2-dimethyl-propyl]amino]-4-oxo-butyl]phenoxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[4-[4-[[(1S)-1-[(2S,4R)-4-hydroxy-2-[[(1R)-2,2,2-trifluoro-1-[4-(4-methylthiazol-5-yl)phenyl]ethyl]carbamoyl]pyrrolidine-1-carbonyl]-2,2-dimethyl-propyl]amino]-4-oxo-butyl]phenoxy]-2-methyl-phenyl]pyridine-2-carboxylate (40 mg, 32.38 μmol, 1.0 equiv.) in TFA (1 mL) and DCM (1 mL) was stirred at 25° C. for 12 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[4-[4-[[(1S)-1-[(2S,4R)-4-hydroxy-2-[[(1R)-2,2,2-trifluoro-1-[4-(4-methylthiazol-5-yl)phenyl]ethyl]carbamoyl]pyrrolidine-1-carbonyl]-2,2-dimethyl-propyl]amino]-4-oxo-butyl]phenoxy]-2-methyl-phenyl]pyridine-2-carboxylic acid (10.3 mg, 8.4 μmol, 25.9% yield, 96.1% purity) as a white solid.

MS (ESI) m/z: 590.6 [M/2+H]⁺.

¹H NMR (400 MHz, DMSO-d₆) δ=9.29 (d, J=9.6 Hz, 1H), 9.02 (s, 1H), 8.02 (d, J=8.0 Hz, 1H), 7.91 (d, J=9.2 Hz, 1H), 7.78 (d, J=8.0 Hz, 1H), 7.66-7.61 (m, 3H), 7.55 (d, J=8.4 Hz, 2H), 7.52-7.48 (m, 1H), 7.47-7.42 (m, 2H), 7.39-7.32 (m, 2H), 7.18-7.11 (m, 3H), 6.96 (d, J=8.8 Hz, 1H), 6.89 (d, J=7.2 Hz, 1H), 6.85-6.78 (m, 3H), 5.85 (t, J=8.4 Hz, 1H), 5.23-5.07 (m, 1H), 4.99 (s, 2H), 4.60-4.52 (m, 2H), 4.29 (s, 1H), 3.92 (t, J=5.6 Hz, 2H), 3.63 (s, 2H), 3.03 (t, J=5.6 Hz, 2H), 2.88 (q, J=7.6 Hz, 1H), 2.47 (s, 3H), 2.25 (d, J=6.4, 14.4 Hz, 2H), 2.21-2.12 (m, 2H), 1.91 (s, 3H), 1.85-1.64 (m, 2H), 1.23 (s, 1H), 0.94 (s, 9H).

### Example 22. Preparation of Compound 121a

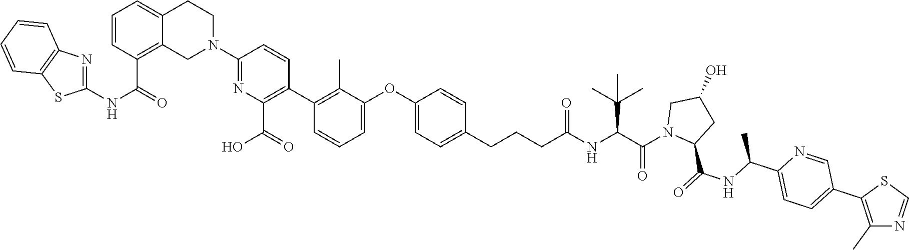 [SMILES: Cc1ncsc1-c1ccc([C@H](C)NC(=O)[C@@H]2C[C@@H](O)CN2C(=O)[C@@H](NC(=O)CCCc2ccc(Oc3cccc(-c4ccc(N5CCc6cccc(C(=O)Nc7nc8ccccc8s7)c6C5)nc4C(=O)O)c3C)cc2)C(C)(C)C)nc1]

### 6-[8-(1,3-benzothiazol-2-ylcarbamoyl)-3,4-dihydro-1H-isoquinolin-2-yl]-3-[3-[4-[4-[[(1S)-1-[(2S,4R)-4-hydroxy-2-[[(1S)-1-[5-(4-methylthiazol-5-yl)-2-pyridyl]ethyl]carbamoyl]pyrrolidine-1-carbonyl]-2,2-dimethyl-propyl]amino]-4-oxo-butyl]phenoxy]-2-methyl-phenyl]pyridine-2-carboxylic acid

### Step A. Procedure for Preparation of tert-butyl N-[(1S)-1-[(2S,4R)-2-[[(1S)-1-(5-bromo-2-pyridyl)ethyl]carbamoyl]-4-hydroxy-pyrrolidine-1-carbonyl]-2,2-dimethyl-propyl]carbamate

A mixture of (1S)-1-(5-bromo-2-pyridyl)ethanamine (500 mg, 2.49 mmol, 1.0 equiv.), (2S,4R)-1-[(2S)-2-(tert-butoxycarbonylamino)-3,3-dimethyl-butanoyl]-4-hydroxy-pyrrolidine-2-carboxylic acid (1.11 g, 3.23 mmol, 1.3 equiv.), HATU (1.13 g, 2.98 mmol, 1.2 equiv.), and DIEA (482.10 mg, 3.73 mmol, 649.73 μL, 1.5 equiv.) in DMF (5 mL) was stirred at 25° C. for 16 hours. The reaction mixture was diluted with water (20 mL) and extracted with ethyl acetate 60 mL (20 mL×3). The combined organic layers were washed with brine 20 mL (10 mL×2), dried over sodium sulfate, filtered, and concentrated under reduced pressure to give a residue. The residue was purified by flash silica gel chromatography (eluent of 81% ethyl acetate/petroleum ether) to give tert-butyl N-[(1S)-1-[(2S,4R)-2-[[(1S)-1-(5-bromo-2-pyridyl)ethyl]carbamoyl]-4-hydroxy-pyrrolidine-1-carbonyl]-2,2-dimethyl-propyl]carbamate (1.2 g, 1.9 mmol, 79.5% yield, 87% purity) as light yellow oil.

MS (ESI) m/z: 527.3 [M+H]⁺.

¹H NMR (400 MHz, DMSO-d₆). δ=8.64-8.58 (m, 1H), 8.39 (d, J=7.6 Hz, 1H), 8.00-7.94 (m, 2H), 7.29 (d, J=8.4 Hz, 1H), 5.12 (s, 1H), 4.84 (q, J=7.2 Hz, 1H), 4.45 (t, J=8.0 Hz, 1H), 4.30-4.23 (m, 1H), 4.03 (q, J=7.2 Hz, 2H), 3.64-3.50 (m, 2H), 2.89 (s, 2H), 1.39-1.35 (m, 12H), 0.92 (s, 9H).

### Step B. Procedure for Preparation of tert-butyl N-[(1S)-1-[(2S,4R)-4-hydroxy-2-[[(1S)-1-[5-(4-methylthiazol-5-yl)-2-pyridyl]ethyl]carbamoyl]pyrrolidine-1-carbonyl]-2,2-dimethyl-propyl]carbamate

A mixture of tert-butyl N-[(1S)-1-[(2S,4R)-2-[[(1S)-1-(5-bromo-2-pyridyl)ethyl]carbamoyl]-4-hydroxy-pyrrolidine-1-carbonyl]-2,2-dimethyl-propyl]carbamate (500 mg, 947.96 μmol, 1.0 equiv.), 4-methylthiazole (187.99 mg, 1.90 mmol, 172.47 μL, 2.0 equiv.), Pd(OAc)₂ (21.28 mg, 94.80 μmol, 0.1 equiv.), and KOAc (186.07 mg, 1.90 mmol, 2.0 equiv.) in DMA (5 mL) was degassed and purged with N₂ three times, and then the mixture was stirred at 130° C. for 4 hours under N₂ atmosphere. The reaction mixture was diluted with water (20 mL) and extracted with ethyl acetate 60 mL (20 mL×3). The combined organic layers were washed with brine 20 mL (10 mL×2), dried over sodium sulfate, filtered, and concentrated under reduced pressure to give a residue. The residue was purified by flash silica gel chromatography (eluent of 10% MeOH/DCM) to give tert-butyl N-[(1S)-1-[(2S,4R)-4-hydroxy-2-[[(1S)-1-[5-(4-methylthiazol-5-yl)-2-pyridyl]ethyl]carbamoyl]pyrrolidine-1-carbonyl]-2,2-dimethyl-propyl]carbamate (200 mg, 250.8 μmol, 26.4% yield, 68.4% purity) as a brown solid.

MS (ESI) m/z: 546.3 [M+H]⁺.

¹H NMR (400 MHz, DMSO-d₆) δ=8.62 (d, J=2.0 Hz, 1H), 8.40 (d, J=7.6 Hz, 1H), 7.88 (dd, J=2.4, 8.0 Hz, 1H), 7.41 (d, J=8.0 Hz, 1H), 6.59-6.27 (m, 1H), 5.13 (d, J=3.2 Hz, 1H), 4.98-4.87 (m, 1H), 4.60-4.39 (m, 1H), 4.34-4.25 (m, 1H), 4.14 (d, J=9.2 Hz, 1H), 4.03 (q, J=7.2 Hz, 1H), 2.46 (s, 2H), 1.99 (s, 1H), 1.40-1.32 (m, 9H), 1.17 (t, J=7.2 Hz, 1H), 0.93 (s, 8H).

### Step C. Procedure for Preparation of (2S,4R)-1-[(2S)-2-amino-3,3-dimethyl-butanoyl]-4-hydroxy-N-[(1S)-1-[5-(4-methylthiazol-5-yl)-2-pyridyl]ethyl]pyrrolidine-2-carboxamide

A mixture of tert-butyl N-[(1S)-1-[(2S,4R)-4-hydroxy-2-[[(1S)-1-[5-(4-methylthiazol-5-yl)-2-pyridyl]ethyl]carbamoyl]pyrrolidine-1-carbonyl]-2,2-dimethyl-propyl]carbamate (200.00 mg, 366.51 μmol, 1.0 equiv.) in HCl/dioxane (2 mL) and DCM (1 mL) was stirred at 25° C. for 16 hours. To the reaction mixture was added DCM (10 mL), then the mixture was filtered to get a filter cake and concentrated under reduced pressure to give (2S,4R)-1-[(2S)-2-amino-3,3-dimethyl-butanoyl]-4-hydroxy-N-[(1S)-1-[5-(4-methylthiazol-5-yl)-2-pyridyl]ethyl]pyrrolidine-2-carboxamide (200 mg, crude) was obtained as brown oil.

MS (ESI) m/z: 446.2 [M+H]⁺.

### Step D. Procedure for Preparation of tert-butyl 6-[8-(1,3-benzothiazol-2-ylcarbamoyl)-3,4-dihydro-1H-isoquinolin-2-yl]-3-[3-[4-[4-[[(1S)-1-[(2S,4R)-4-hydroxy-2-[[(1S)-1-[5-(4-methylthiazol-5-yl)-2-pyridyl]ethyl]carbamoyl]pyrrolidine-1-carbonyl]-2,2-dimethyl-propyl]amino]-4-oxo-butyl]phenoxy]-2-methyl-phenyl]pyridine-2-carboxylate

A mixture of 4-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]phenyl]butanoic acid (80 mg, 105.98 μmol, 1.0 equiv.), (2S,4R)-1-[(2S)-2-amino-3,3-dimethyl-butanoyl]-4-hydroxy-N-[(1S)-1-[5-(4-methylthiazol-5-yl)-2-pyridyl]ethyl]pyrrolidine-2-carboxamide (56.66 mg, 127.17 μmol, 1.2 equiv.), DIEA (41.09 mg, 317.93 μmol, 55.38 μL, 3.0 equiv.), and HATU (60.44 mg, 158.96 μmol, 1.5 equiv.) in DMF (2 mL) was stirred at 25° C. for 16 hours. To the reaction mixture was added water (10 mL), which was then filtered to give a filter cake. The residue was purified by prep-TLC (SiO₂, DCM:MeOH=10:1) to give tert-butyl 6-[8-(1,3-benzothiazol-2-ylcarbamoyl)-3,4-dihydro-1H-isoquinolin-2-yl]-3-[3-[4-[4-[[(1S)-1-[(2S,4R)-4-hydroxy-2-[[(1S)-1-[5-(4-methylthiazol-5-yl)-2-pyridyl]ethyl]carbamoyl]pyrrolidine-1-carbonyl]-2,2-dimethyl-propyl]amino]-4-oxo-butyl]phenoxy]-2-methyl-phenyl]pyridine-2-carboxylate (50 mg, 24.7 μmol, 23.3% yield, 58.4% purity) as a yellow oil.

MS (ESI) m/z: 592.2 [M12+H]⁺.

### Step E. Procedure for Preparation of 6-[8-(1,3-benzothiazol-2-ylcarbamoyl)-3,4-dihydro-1H-isoquinolin-2-yl]-3-[3-[4-[4-[[(1S)-1-[(2S,4R)-4-hydroxy-2-[[(1S)-1-[5-(4-methylthiazol-5-yl)-2-pyridyl]ethyl]carbamoyl]pyrrolidine-1-carbonyl]-2,2-dimethyl-propyl]amino]-4-oxo-butyl]phenoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[4-[4-[[(1S)-1-[(2S,4R)-4-hydroxy-2-[[(1S)-1-[5-(4-methylthiazol-5-yl)-2-pyridyl]ethyl]carbamoyl]pyrrolidine-1-carbonyl]-2,2-dimethyl-propyl]amino]-4-oxo-butyl]phenoxy]-2-methyl-phenyl]pyridine-2-carboxylate (50 mg, 42.28 μmol, 1.0 equiv.) in DCM (0.8 mL) and TFA (0.8 mL) was stirred at 25° C. for 12 hours. The reaction mixture was concentrated under reduced pressure to give a residue. The crude product was purified by reversed-phase HPLC to give 6-[8-(1,3-benzothiazol-2-ylcarbamoyl)-3,4-dihydro-1H-isoquinolin-2-yl]-3-[3-[4-[4-[[(1S)-1-[(2S,4R)-4-hydroxy-2-[[(1S)-1-[5-(4-methylthiazol-5-yl)-2-pyridyl]ethyl]carbamoyl]pyrrolidine-1-carbonyl]-2,2-dimethyl-propyl]amino]-4-oxo-butyl]phenoxy]-2-methyl-phenyl]pyridine-2-carboxylic acid (17.6 mg, 15.0 μmol, 35.5% yield, 96.0% purity) as yellow gum.

MS (ESI) m/z: 564.2 [M12+H]⁺.

¹H NMR (400 MHz, DMSO-d₆) δ=9.09 (s, 1H), 8.64-8.61 (m, 1H), 8.35 (s, 1H), 8.00 (d, J=2.4 Hz, 1H), 7.92-7.82 (m, 2H), 7.81-7.75 (m, 1H), 7.62 (d, J=7.2 Hz, 1H), 7.55-7.49 (m, 1H), 7.49-7.40 (m, 3H), 7.40-7.30 (m, 2H), 7.20-7.11 (m, 3H), 7.02-6.96 (m, 1H), 6.92-6.86 (m, 1H), 6.85-6.77 (m, 3H), 4.99 (s, 2H), 4.96-4.91 (m, 1H), 4.53 (d, J=9.2 Hz, 1H), 4.46 (t, J=8.0 Hz, 1H), 4.31-4.26 (m, 1H), 3.93 (t, J=5.6 Hz, 2H), 3.62 (s, 2H), 3.03 (d, J=10.4 Hz, 2H), 2.46 (s, 4H), 2.25 (d, J=6.8 Hz, 1H), 2.21-2.12 (m, 1H), 2.02 (s, 1H), 1.93-1.88 (m, 3H), 1.86 (s, 1H), 1.76 (d, J=8.0 Hz, 1H), 1.41 (d, J=7.2 Hz, 3H), 0.97-0.90 (m, 9H).

### Example 23. Preparation of Compound 122a

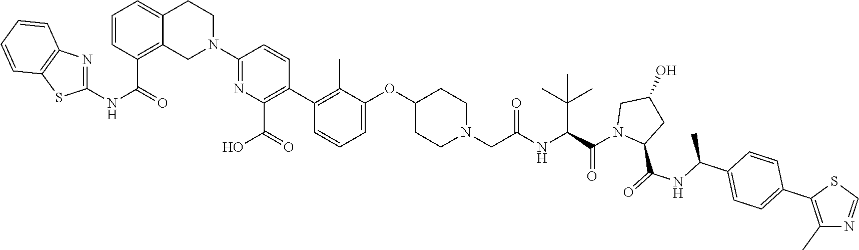 [SMILES: Cc1ncsc1-c1ccc([C@H](C)NC(=O)[C@@H]2C[C@@H](O)CN2C(=O)[C@@H](NC(=O)CN2CCC(Oc3cccc(-c4ccc(N5CCc6cccc(C(=O)Nc7nc8ccccc8s7)c6C5)nc4C(=O)O)c3C)CC2)C(C)(C)C)cc1]

### 6-[8-(1,3-benzothiazol-2-ylcarbamoyl)-3,4-dihydro-1H-isoquinolin-2-yl]-3-[3-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]oxy]-2-methyl-phenyl]pyridine-2-carboxylic acid

### Step A. Procedure for Preparation of tert-butyl 4-(3-bromo-2-methyl-phenoxy)piperidine-1-carboxylate

A mixture of tert-butyl 4-hydroxypiperidine-1-carboxylate (4.2 g, 20.87 mmol, 1.00 equiv.), 3-bromo-2-methyl-phenol (4.29 g, 22.96 mmol, 1.10 equiv.), and 2-(tributyl-λ⁵-phosphanylidene)acetonitrile (6.04 g, 25.04 mmol, 1.20 equiv.) in toluene (50 mL) was degassed and purged with N₂ three times, and then the mixture was stirred at 120° C. for 18 hours under N₂ atmosphere. The residue was purified by flash silica gel chromatography (eluent of 0˜15% ethyl acetate/petroleum ether) to give tert-butyl 4-(3-bromo-2-methyl-phenoxy)piperidine-1-carboxylate (5.2 g, 56.1 μmol, 76.2% yield, 91.1% purity) as a yellow oil.

MS (ESI) m/z: 503.3 [M+H]⁺.

### Step B. Procedure for Preparation of 4-(3-bromo-2-methyl-phenoxy)piperidine

A solution of tert-butyl 4-(3-bromo-2-methyl-phenoxy)piperidine-1-carboxylate (5 g, 13.50 mmol, 1.0 equiv.) in HCl/dioxane (60 mL) was stirred at 25° C. for 2 hours. The reaction mixture was then filtered and concentrated under reduced pressure to give 4-(3-bromo-2-methyl-phenoxy)piperidine (4 g, crude, HCl) as a white solid.

MS (ESI) m/z: 270.1 [M+H]⁺.

### Step C. Procedure for Preparation of ethyl 2-[4-(3-bromo-2-methyl-phenoxy)-1-piperidyl]acetate

To a solution of 4-(3-bromo-2-methyl-phenoxy)piperidine (500 mg, 1.85 mmol, 1.00 equiv.) and ethyl 2-bromoacetate (293.62 mg, 1.76 mmol, 194.45 μL, 0.95 equiv.) in MeCN (5 mL) was added K₂CO₃ (767.34 mg, 5.55 mmol, 3.00 equiv.). The mixture was stirred at 25° C. for 2 hours. The reaction mixture was concentrated under reduced pressure to give ethyl 2-[4-(3-bromo-2-methyl-phenoxy)-1-piperidyl]acetate (550 mg, crude) as a yellow oil.

MS (ESI) m/z: 358.1 [M+H]⁺

### Step D. Procedure for Preparation of tert-butyl 6-[8-(1,3-benzothiazol-2-ylcarbamoyl)-3,4-dihydro-1H-isoquinolin-2-yl]-3-[3-[[1-(2-ethoxy-2-oxo-ethyl)-4-piperidyl]oxy]-2-methyl-phenyl]pyridine-2-carboxylate

Ethyl 2-[4-(3-bromo-2-methyl-phenoxy)-1-piperidyl]acetate (280 mg, 785.96 μmol, 1.00 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (481.43 mg, 785.96 μmol, 1.00 equiv.), Ad₂nBuP Pd G₃ (cataCXium® A Pd G₃) (114.48 mg, 157.19 μmol, 0.20 equiv.) and K₂CO₃ (162.94 mg, 1.18 mmol, 1.50 equiv.) were taken up into a microwave tube in dioxane (5 mL) and water (0.5 mL). The sealed tube was heated at 100° C. for 1 hour in a microwave. The reaction mixture was then concentrated under reduced pressure to give tert-butyl 6-[8-(1,3-benzothiazol-2-ylcarbamoyl)-3,4-dihydro-1H-isoquinolin-2-yl]-3-[3-[[1-(2-ethoxy-2-oxo-ethyl)-4-piperidyl]oxy]-2-methyl-phenyl]pyridine-2-carboxylate (400 mg, crude) as a yellow oil.

MS (ESI) m/z: 762.4 [M+H]⁺

### Step E. Procedure for Preparation of 2-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-1-piperidyl]acetic acid

To a solution of tert-butyl 6-[8-(1,3-benzothiazol-2-ylcarbamoyl)-3,4-dihydro-1H-isoquinolin-2-yl]-3-[3-[[1-(2-ethoxy-2-oxo-ethyl)-4-piperidyl]oxy]-2-methyl-phenyl]pyridine-2-carboxylate (400 mg, 524.99 μmol, 1.00 equiv.) in THE (3 mL) and water (1 mL) was added LiOH·H₂O (33.05 mg, 787.48 μmol, 1.50 equiv.). The mixture was stirred at 25° C. for 18 hours. The reaction mixture was partitioned between DCM (30 mL) and MeOH (3 mL). The organic phase was separated, washed with water (15 mL), dried over sodium sulfate, filtered, and concentrated under reduced pressure to give a residue. The residue was purified by prep-TLC (SiO₂, DCM:MeOH=10:1) to give 2-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-1-piperidyl]acetic acid (340 mg, 423.8 μmol, 80.7% yield, 91.4% purity) as a yellow oil

MS (ESI) m/z: 734.4 [M+H]⁺

### Step F. Procedure for Preparation of tert-butyl 6-[8-(1,3-benzothiazol-2-ylcarbamoyl)-3,4-dihydro-1H-isoquinolin-2-yl]-3-[3-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]oxy]-2-methyl-phenyl]pyridine-2-carboxylate

To a solution of 2-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-1-piperidyl]acetic acid (220 mg, 299.78 μmol, 1.00 equiv.) and (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (199.92 mg, 449.67 μmol, 1.50 equiv.) in pyridine (5 mL) was added EDCI (86.20 mg, 449.67 μmol, 1.50 equiv.). The mixture was stirred at 25° C. for 2 hours. The reaction mixture was concentrated under reduced pressure to give a residue. The residue was purified by prep-TLC (SiO₂, DCM:MeOH=10:1) to give tert-butyl 6-[8-(1,3-benzothiazol-2-ylcarbamoyl)-3,4-dihydro-1H-isoquinolin-2-yl]-3-[3-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]oxy]-2-methyl-phenyl]pyridine-2-carboxylate (250 mg, 201.5 μmol, 67.2% yield, 93.5% purity) as a yellow oil.

MS (ESI) m/z: 734.4 [M+H]

### Step G. Procedure for Preparation of 6-[8-(1,3-benzothiazol-2-ylcarbamoyl)-3,4-dihydro-1H-isoquinolin-2-yl]-3-[3-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]oxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]oxy]-2-methyl-phenyl]pyridine-2-carboxylate (180 mg, 155.11 μmol, 1.00 equiv.) in DCM (2 mL) was added TFA (17.69 mg, 155.11 μmol, 11.48 μL, 1.00 equiv.). The mixture was stirred at 25° C. for 2 hours. The reaction mixture was concentrated under reduced pressure to give a residue which was purified by reversed-phase HPLC to give 6-[8-(1,3-benzothiazol-2-ylcarbamoyl)-3,4-dihydro-1H-isoquinolin-2-yl]-3-[3-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]oxy]-2-methyl-phenyl]pyridine-2-carboxylic acid (28.2 mg, 25.5 μmol, 16.5% yield, 99.7% purity) as a white solid

MS (ESI) m/z: 553.1 [M/2+H]⁺

¹H NMR (400 MHz, DMSO-d₆) δ=8.98 (s, 1H), 8.46-8.42 (m, 1H), 8.17 (s, 1H), 8.05-8.00 (m, 1H), 7.78 (d, J=7.6 Hz, 2H), 7.64-7.60 (m, 1H), 7.49-7.41 (m, 5H), 7.39-7.33 (m, 4H), 7.10-7.05 (m, 1H), 6.96-6.92 (m, 2H), 6.67-6.61 (m, 1H), 4.98 (s, 2H), 4.91-4.86 (m, 1H), 4.53-4.40 (m, 3H), 4.25 (s, 1H), 3.94-3.88 (m, 2H), 3.65-3.53 (m, 4H), 3.03 (d, J=5.2 Hz, 2H), 2.97-2.94 (m, 2H), 2.76-2.69 (m, 2H), 2.45 (s, 3H), 2.06-1.94 (m, 3H), 1.92 (s, 3H), 1.79-1.68 (m, 3H), 1.40-1.33 (m, 3H), 1.25-1.20 (m, 1H), 0.94 (s, 9H)

### Example 24. Preparation of Compound 123a

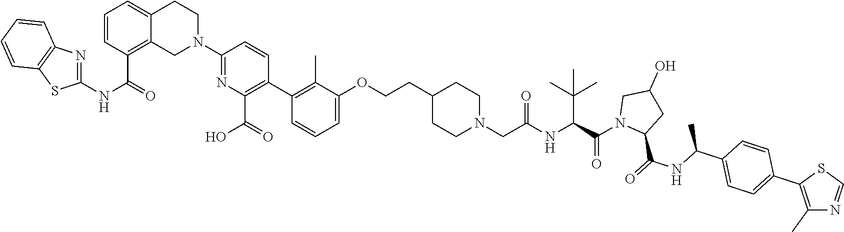 [SMILES: Cc1ncsc1-c1ccc([C@H](C)NC(=O)[C@@H]2CC(O)CN2C(=O)[C@@H](NC(=O)CN2CCC(CCOc3cccc(-c4ccc(N5CCc6cccc(C(=O)Nc7nc8ccccc8s7)c6C5)nc4C(=O)O)c3C)CC2)C(C)(C)C)cc1]

### 6-(8-(benzo[d]thiazol-2-ylcarbamoyl)-3,4-dihydroisoquinolin-2 (1H)-yl)-3-(3-(2-(1-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperidin-4-yl)ethoxy)-2-methylphenyl)picolinic acid

### Step A. Procedure for Preparation of tert-butyl 4-[2-(3-bromo-2-methyl-phenoxy) ethyl]piperidine-1-carboxylate

A mixture of 3-bromo-2-methyl-phenol (2.5 g, 13.37 mmol, 1.0 equiv.), tert-butyl 4-(2-hydroxyethyl)piperidine-1-carboxylate (3.07 g, 13.37 mmol, 1.0 equiv.), and 2-(tributyl-λ⁵-phosphanylidene)acetonitrile (4.84 g, 20.05 mmol, 1.5 equiv.) in toluene (25 mL) was degassed and purged with N₂ three times and stirred at 120° C. for 16 hours under N₂ atmosphere. The reaction mixture was concentrated under reduced pressure to remove solvent and the residue was diluted with water (50 mL), extracted with ethyl acetate 150 mL (50 mL×3), dried over sodium sulfate, filtered, and concentrated under reduced pressure to give a residue. The residue was purified by flash silica gel chromatography (eluent of 0˜10% ethyl acetate/petroleum ether) to give tert-butyl 4-[2-(3-bromo-2-methyl-phenoxy) ethyl]piperidine-1-carboxylate (5.1 g, 12.6 mmol, 94.5% yield, 98.7% purity) was obtained as a yellow oil.

MS (ESI) m/z: 343.9 [M−56+H]⁺

¹H NMR (400 MHz, DMSO-d₆) δ=7.20-7.02 (m, 2H), 7.01 (d, J=8.0 Hz, 1H), 4.06 (t, J=5.6 Hz, 2H), 3.97 (d, J=11.6 Hz, 2H), 2.73 (s, 2H), 2.27 (s, 3H), 1.73-1.70 (m, 5H), 1.43 (s, 9H), 1.08-1.11 (m, 1H), 0.89-0.87 (m, 1H).

### Step B. Procedure for Preparation of 4-[2-(3-bromo-2-methyl-phenoxy)ethyl]piperidine

A solution of tert-butyl 4-[2-(3-bromo-2-methyl-phenoxy) ethyl]piperidine-1-carboxylate (4.8 g, 12.05 mmol, 1.0 equiv.) in dioxane (8 mL) and HCl/dioxane (20 mL) was stirred at 25° C. for 1 hour. The mixture was filtered to give a filter cake which was triturated with DCM (20 mL) at 25° C. for 10 min to give 4-[2-(3-bromo-2-methyl-phenoxy)ethyl]piperidine (3.7 g, 11.0 mmol, 91.7% yield) as a white solid.

MS (ESI) m/z: 300.1 [M+H]⁺

### Step C. Procedure for Preparation of ethyl 2-[4-[2-(3-bromo-2-methyl-phenoxy)ethyl]-1-piperidyl]acetate

A mixture of 4-[2-(3-bromo-2-methyl-phenoxy)ethyl]piperidine (1.0 g, 2.99 mmol, 1.0 equiv., HCl), ethyl 2-bromoacetate (498.99 mg, 2.99 mmol, 330.46 μL, 1.0 equiv.), and K₂CO₃ (1.24 g, 8.96 mmol, 3.0 equiv.) in MeCN (10 mL) was degassed and purged with N₂ three times, and stirred at 60° C. for 2 hours. The mixture solution was then quenched with water (120 mL) and extracted with ethyl acetate 300 mL (100 mL×3). The organic phase washed with brine 200 mL (100 mL×2), dried with sodium sulfate, filtered, and concentrated under reduced pressure. The residue was purified by flash silica gel chromatography (eluent of 0˜50% ethyl acetate/petroleum ether) to give ethyl 2-[4-[2-(3-bromo-2-methyl-phenoxy)ethyl]-1-piperidyl]acetate (0.8 g, 2.0 mmol, 69.7% yield) as yellow oil.

MS (ESI) m/z: 385.9 [M+H]⁺

¹H NMR (400 MHz, DMSO-d₆) δ=7.16-7.07 (m, 2H), 6.98 (d, J=8.0 Hz, 1H), 4.00-4.08 (m, 4H), 3.16 (s, 2H), 2.65 (d, J=11.2 Hz, 2H), 2.22 (s, 3H), 2.13-2.12 (m, 2H), 1.69-1.66 (m, 4H), 1.44-1.31 (m, 1H), 1.20-1.17 (m, 5H).

### Step D. Procedure for Preparation of tert-butyl 6-[8-(1,3-benzothiazol-2-ylcarbamoyl)-3,4-dihydro-1H-isoquinolin-2-yl]-3-[3-[2-[1-(2-ethoxy-2-oxo-ethyl)-4-piperidyl]ethoxy]-2-methyl-phenyl]pyridine-2-carboxylate

A mixture of ethyl 2-[4-[2-(3-bromo-2-methyl-phenoxy)ethyl]-1-piperidyl]acetate (500 mg, 1.30 mmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (796.95 mg, 1.30 mmol, 1.0 equiv.), Ad₂nBuP Pd G₃ (cataCXium® A Pd G₃) (47.38 mg, 65.05 μmol, 0.05 equiv.), and K₂CO₃ (539.44 mg, 3.90 mmol, 3.0 equiv.) in dioxane (10 mL) and water (0.5 mL) was degassed and purged with N₂ three times, and then the mixture was stirred at 100° C. for 16 hours under N₂ atmosphere. The reaction mixture was concentrated under reduced pressure to provide a residue. The residue was diluted with water (50 mL) and extracted with ethyl acetate 150 mL (50 mL×3) and the combined organic layers were dried over sodium sulfate, filtered, and concentrated under reduced pressure to give a residue which was purified by flash silica gel chromatography (eluent of 80˜100% ethyl acetate/petroleum ether) to give tert-butyl 6-[8-(1,3-benzothiazol-2-ylcarbamoyl)-3,4-dihydro-1H-isoquinolin-2-yl]-3-[3-[2-[1-(2-ethoxy-2-oxo-ethyl)-4-piperidyl]ethoxy]-2-methyl-phenyl]pyridine-2-carboxylate (769 mg, 836.7 μmol, 64.3% yield, 85.9% purity) as a yellow solid.

MS (ESI) m/z: 790.4 [M+H]⁺

### Step E. Procedure for Preparation of 2-[4-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethyl]-1-piperidyl]acetic acid

To a solution of tert-butyl 6-[8-(1,3-benzothiazol-2-ylcarbamoyl)-3,4-dihydro-1H-isoquinolin-2-yl]-3-[3-[2-[1-(2-ethoxy-2-oxo-ethyl)-4-piperidyl]ethoxy]-2-methyl-phenyl]pyridine-2-carboxylate (300 mg, 379.76 μmol, 1.0 equiv.) in MeOH (5 mL) and water (3 mL) was added LiOH·H₂O (63.74 mg, 1.52 mmol, 4.0 equiv.). The mixture was stirred at 25° C. for 2 hours. The reaction mixture was concentrated under reduced pressure to remove solvent. Then water (30 mL) was added to the mixture and the pH adjusted to 3-4 by 1N HCl. The mixture was filtered to give 2-[4-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethyl]-1-piperidyl]acetic acid (185.5 mg, 193.7 μmol, 51.0% yield) as a yellow solid.

¹H NMR (400 MHz, DMSO-d₆) δ=12.8-12.9 (m, 1H), 8.02 (d, J=7.6 Hz, 1H), 7.78 (d, J=8.0 Hz, 1H), 7.61 (d, J=7.2 Hz, 1H), 7.49-7.30 (m, 5H), 7.14-7.07 (m, 1H), 6.99-6.88 (m, 2H), 6.59 (d, J=7.6 Hz, 1H), 4.98 (s, 2H), 3.89-3.82 (m, 7H), 3.05-3.02 (m, 5H), 1.99 (s, 2H), 1.98 (s, 3H), 1.92-1.67 (m, 2H), 1.24-1.18 (m, 2H), 1.02 (s, 9H).

### Step F. Procedure for Preparation of tert-butyl 6-[8-(1,3-benzothiazol-2-ylcarbamoyl)-3,4-dihydro-1H-isoquinolin-2-yl]-3-[3-[2-[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]ethoxy]-2-methyl-phenyl]pyridine-2-carboxylate

To a solution of 2-[4-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethyl]-1-piperidyl]acetic acid (45 mg, 59.06 μmol, 1.0 equiv.) in DMF (3 mL) was added HATU (33.69 mg, 88.59 μmol, 1.5 equiv.) and DIEA (22.90 mg, 177.18 μmol, 30.86 μL, 3.0 equiv.). Then (2S, 4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (31.51 mg, 70.87 μmol, 1.2 equiv.) was added. The mixture was stirred at 25° C. for 16 hours. The residue was diluted with water (50 mL) and extracted with ethyl acetate 150 mL (50 mL×3). The combined organic layers dried over sodium sulfate, filtered, and concentrated under reduced pressure to give a residue. The residue was purified by prep-HPLC (TFA condition) to give tert-butyl 6-[8-(1,3-benzothiazol-2-ylcarbamoyl)-3,4-dihydro-1H-isoquinolin-2-yl]-3-[3-[2-[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]ethoxy]-2-methyl-phenyl]pyridine-2-carboxylate (60.0 mg, 37.0 μmol, 62.6% yield, 73.2% purity) as a yellow gum.

MS (ESI) m/z: 595.2 [M/2+1]

### Step G. Procedure for Preparation of 6-(8-(benzo[d]thiazol-2-ylcarbamoyl)-3,4-dihydroisoquinolin-2 (1H)-yl)-3-(3-(2-(1-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperidin-4-yl)ethoxy)-2-methylphenyl)picolinic acid

A solution of tert-butyl 6-[8-(1,3-benzothiazol-2-ylcarbamoyl)-3,4-dihydro-1H-isoquinolin-2-yl]-3-[3-[2-[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]ethoxy]-2-methyl-phenyl]pyridine-2-carboxylate (60 mg, 50.48 μmol, 1.0 equiv.) and TFA (2 mL) in DCM (2 mL) was stirred at 25° C. for 16 hours. The reaction mixture was concentrated under reduced pressure to remove solvent. The residue was purified by prep-HPLC to give 6-(8-(benzo[d]thiazol-2-ylcarbamoyl)-3,4-dihydroisoquinolin-2 (1H)-yl)-3-(3-(2-(1-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperidin-4-yl)ethoxy)-2-methylphenyl)picolinic acid (35.9 mg, 29.0 μmol, 57.6% yield) as a yellow solid.

MS (ESI) m/z: 1132.7 [M+H]⁺

¹H NMR (400 MHz, DMSO-d₆) δ=8.98 (s, 1H), 8.44 (d, J=7.6 Hz, 1H), 8.03 (d, J=7.6 Hz, 1H), 7.79 (d, J=8.4 Hz, 2H), 7.80-7.77 (m, 1H), 7.47-7.47 (m, 9H), 7.09 (t, J=7.6 Hz, 1H), 6.92-6.81 (m, 2H), 6.64 (d, J=7.6 Hz, 1H), 4.98 (s, 2H), 4.90-4.88 (m, 1H), 4.44-4.29 (m, 1H), 4.28 (s, 1H), 3.92-3.59 (m, 4H), 3.58-3.57 (m, 3H), 3.08-3.00 (m, 4H), 2.99-2.82 (m, 4H), 2.45 (s, 3H), 1.90 (s, 3H), 1.76-1.72 (m, 6H), 1.38-1.37 (m, 3H), 1.26-1.24 (m, 4H), 0.94 (s, 9H).

### Example 25. Preparation of Compound 124a

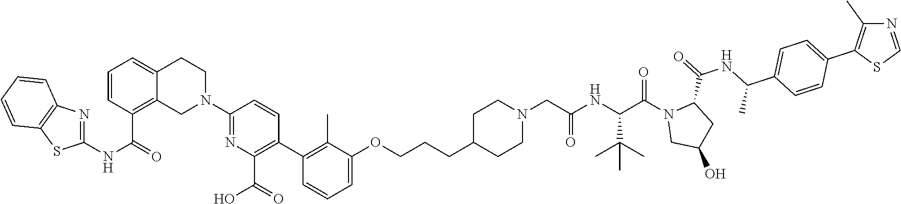 [SMILES: Cc1ncsc1-c1ccc([C@H](C)NC(=O)[C@@H]2C[C@@H](O)CN2C(=O)[C@@H](NC(=O)CN2CCC(CCCOc3cccc(-c4ccc(N5CCc6cccc(C(=O)Nc7nc8ccccc8s7)c6C5)nc4C(=O)O)c3C)CC2)C(C)(C)C)cc1]

### 6-[8-(1,3-benzothiazol-2-ylcarbamoyl)-3,4-dihydro-1H-isoquinolin-2-yl]-3-[3-[3-[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]propoxy]-2-methyl-phenyl]pyridine-2-carboxylic acid

### Step A. Procedure for Preparation of tert-butyl 4-[3-(3-bromo-2-methyl-phenoxy)propyl]piperidine-1-carboxylate

A mixture of 3-bromo-2-methyl-phenol (500 mg, 2.67 mmol, 1.0 equiv.), tert-butyl 4-(3-hydroxypropyl) piperidine-1-carboxylate (715.58 mg, 2.94 mmol, 1.1 equiv.), and 2-(tributyl-λ5-phosphanylidene)acetonitrile (967.82 mg, 4.01 mmol, 1.5 equiv.) in toluene (5 mL) was degassed and purged with N₂ three times. The mixture was then stirred at 120° C. for 2 hours under N₂ atmosphere. The reaction mixture was concentrated, and the residue was purified by flash silica gel chromatography (eluent of 9% ethyl acetate/petroleum ether) to give tert-butyl 4-[3-(3-bromo-2-methyl-phenoxy)propyl]piperidine-1-carboxylate (1.0 g, 2.5 mmol, 95.2% yield) as a brown oil.

¹H NMR (400 MHz, DMSO-d₆) δ=7.16-7.12 (m, 1H), 7.11-7.05 (m, 1H), 6.95 (d, J=7.6 Hz, 1H), 3.98-3.88 (m, 4H), 2.67 (s, 2H), 2.22 (s, 3H), 1.78-1.70 (m, 2H), 1.64 (d, J=11.6 Hz, 2H), 1.41-1.32 (m, 12H), 1.02-0.91 (m, 2H).

### Step B. Procedure for Preparation of 4-[3-(3-bromo-2-methyl-phenoxy)propyl]piperidine

A solution of tert-butyl 4-[3-(3-bromo-2-methyl-phenoxy)propyl]piperidine-1-carboxylate (1 g, 2.43 mmol, 1.0 equiv.) in HCl/dioxane (10 mL) was stirred at 25° C. for 2 hours. The mixture was then concentrated to give 4-[3-(3-bromo-2-methyl-phenoxy)propyl]piperidine (820 mg, crude) as a white solid.

¹H NMR (400 MHz, DMSO-d₆) δ=7.17-7.13 (m, 1H), 7.12-7.06 (m, 1H), 6.96 (d, J=8.0 Hz, 1H), 3.97 (t, J=6.4 Hz, 2H), 3.21 (d, J=12.0 Hz, 2H), 2.80 (t, J=11.6 Hz, 2H), 2.23 (s, 3H), 1.84-1.70 (m, 5H), 1.63-1.48 (m, 1H), 1.41-1.28 (m, 4H).

### Step C. Procedure for Preparation of ethyl 2-[4-[3-(3-bromo-2-methyl-phenoxy)propyl]-1-piperidyl]acetate

A mixture of 4-[3-(3-bromo-2-methyl-phenoxy)propyl]piperidine (400 mg, 1.15 mmol, 1 eq, HCl), ethyl 2-bromoacetate (181.99 mg, 1.09 mmol, 120.52 μL, 0.95 equiv.), and K₂CO₃ (475.61 mg, 3.44 mmol, 3 equiv.) in MeCN (4 mL) was degassed and purged with N₂ three times. The mixture was then stirred at 60° C. for 1 hour under N₂ atmosphere. The residue was concentrated to give ethyl 2-[4-[3-(3-bromo-2-methyl-phenoxy)propyl]-1-piperidyl]acetate (430 mg, 1.0 mmol, 94.1% yield) as a white solid.

MS (ESI) m/z: 398.1 [M+H]⁺.

¹H NMR (400 MHz, DMSO-d₆) δ=7.16-7.12 (m, 1H), 7.11-7.06 (m, 1H), 6.95 (d, J=8.0 Hz, 1H), 4.07 (q, J=7.2 Hz, 2H), 3.96 (t, J=6.4 Hz, 2H), 3.15 (s, 2H), 2.79 (d, J=11.2 Hz, 2H), 2.23 (s, 3H), 2.14-2.07 (m, 2H), 1.77-1.70 (m, 2H), 1.63 (d, J=11.6 Hz, 2H), 1.38-1.32 (m, 2H), 1.29-1.19 (m, 1H), 1.18 (t, J=7.2 Hz, 4H), 1.13-1.08 (m, 1H).

### Step D. Procedure for Preparation of tert-butyl 6-[8-(1,3-benzothiazol-2-ylcarbamoyl)-3,4-dihydro-1H-isoquinolin-2-yl]-3-[3-[3-[1-(2-ethoxy-2-oxoethyl)-4-piperidyl]propoxy]-2-methyl-phenyl]pyridine-2-carboxylate

Ethyl 2-[4-[3-(3-bromo-2-methyl-phenoxy)propyl]-1-piperidyl]acetate (200 mg, 502.09 μmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (307.55 mg, 502.09 μmol, 1.0 equiv.), Ad₂nBuP Pd G₃ (cataCXium® A Pd G₃) (73.13 mg, 100.42 μmol, 0.2 equiv.), and K₂CO₃ (104.09 mg, 753.14 μmol, 1.5 equiv.) were taken up into a microwave tube in dioxane (2 mL) and water (0.2 mL). The sealed tube was heated at 100° C. for 1 hour under microwave. The reaction was filtered and concentrated under reduced pressure to give a residue which was purified by flash silica gel chromatography (eluent of 45% ethyl acetate/petroleum ether) to give tert-butyl 6-[8-(1,3-benzothiazol-2-ylcarbamoyl)-3,4-dihydro-1H-isoquinolin-2-yl]-3-[3-[3-[1-(2-ethoxy-2-oxoethyl)-4-piperidyl]propoxy]-2-methyl-phenyl]pyridine-2-carboxylate (230 mg, 286.0 μmol, 56.9% yield) as a white oil.

MS (ESI) m/z: 402.8 [M12+H]⁺.

¹H NMR (400 MHz, DMSO-d₆) δ=12.91-12.78 (m, 1H), 8.02 (d, J=7.6 Hz, 1H), 7.77 (d, J=8.0 Hz, 1H), 7.60 (d, J=7.2 Hz, 1H), 7.48-7.42 (m, 3H), 7.39-7.32 (m, 2H), 7.12-7.07 (m, 1H), 6.92 (dd, J=8.8, 14.8 Hz, 2H), 6.57 (d, J=7.6 Hz, 1H), 4.97 (s, 2H), 4.09-4.00 (m, 4H), 3.96 (t, J=6.0 Hz, 2H), 3.87 (t, J=6.0 Hz, 2H), 3.15 (s, 2H), 3.03 (t, J=6.0 Hz, 2H), 2.79 (d, J=10.4 Hz, 2H), 1.99 (s, 2H), 1.87 (s, 2H), 1.75-1.70 (m, 2H), 1.61 (d, J=11.6 Hz, 2H), 1.37-1.32 (m, 2H), 1.20-1.09 (m, 6H), 1.00 (s, 9H).

### Step E. Procedure for Preparation of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-1-piperidyl]acetic acid

A solution of tert-butyl 6-[8-(1,3-benzothiazol-2-ylcarbamoyl)-3,4-dihydro-1H-isoquinolin-2-yl]-3-[3-[3-[1-(2-ethoxy-2-oxoethyl)-4-piperidyl]propoxy]-2-methyl-phenyl]pyridine-2-carboxylate (230 mg, 286.07 μmol, 1.0 equiv.) and LiOH·H₂O (36.01 mg, 858.20 μmol, 3.0 equiv.) in THE (3 mL) and water (1 mL) was stirred at 25° C. for 2 hours. The reaction mixture was concentrated, and the organic phase was acidified with HCl (lM) to a pH of 2-3. The resulting mixture was filtered and concentrated to give 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-1-piperidyl]acetic acid (210 mg, 270.6 μmol, 94.6% yield) as a yellow solid.

MS (ESI) m/z: 388.7 [M12+H]⁺.

¹H NMR (400 MHz, DMSO-d₆) δ=8.02 (d, J=8.0 Hz, 1H), 7.78 (d, J=8.0 Hz, 1H), 7.60 (d, J=7.6 Hz, 1H), 7.48-7.42 (m, 3H), 7.39-7.32 (m, 2H), 7.12-7.07 (m, 1H), 6.93 (dd, J=8.4, 13.6 Hz, 2H), 6.58 (d, J=7.6 Hz, 1H), 5.75 (s, 1H), 4.98 (s, 1H), 4.05 (s, 2H), 3.97 (t, J=6.0 Hz, 2H), 3.87 (t, J=6.0 Hz, 2H), 3.06-2.97 (m, 5H), 1.90-1.80 (m, 6H), 1.78-1.70 (m, 3H), 1.55-1.47 (m, 3H), 1.44-1.34 (m, 3H), 1.01 (s, 9H)

### Step F. Procedure for Preparation of tert-butyl 6-[8-(1,3-benzothiazol-2-ylcarbamoyl)-3,4-dihydro-1H-isoquinolin-2-yl]-3-[3-[3-[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]propoxy]-2-methyl-phenyl]pyridine-2-carboxylate

To a solution of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-1-piperidyl]acetic acid (100 mg, 128.87 μmol, 1.0 equiv.) and (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (85.94 mg, 193.31 μmol, 1.5 equiv.) in DMF (1 mL) was added HATU (98.00 mg, 257.75 μmol, 2.0 equiv.) and DIEA (49.97 mg, 386.62 μmol, 67.34 μL, 3.0 equiv.). The mixture was stirred at 25° C. for 2 hours. The reaction mixture was treated with water (10 ml), and then the reaction mixture was concentrated under reduced pressure to remove solvent. The organic phase was spin-dried to give tert-butyl 6-[8-(1,3-benzothiazol-2-ylcarbamoyl)-3,4-dihydro-1H-isoquinolin-2-yl]-3-[3-[3-[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]propoxy]-2-methyl-phenyl]pyridine-2-carboxylate (120 mg, 99.7 μmol, 77.4% yield) as a yellow solid.

MS (ESI) m/z: 602.1 [M/2+H]⁺.

¹H NMR (400 MHz, DMSO-d₆) δ=12.86 (s, 1H), 9.00-8.95 (m, 1H), 8.39 (d, J=8.0 Hz, 1H), 8.02 (d, J=8.0 Hz, 1H), 7.78 (d, J=8.0 Hz, 1H), 7.60 (d, J=7.6 Hz, 1H), 7.46-7.42 (m, 5H), 7.40-7.34 (m, 4H), 7.13-7.08 (m, 1H), 6.96-6.90 (m, 2H), 6.60-6.56 (m, 1H), 5.12 (d, J=3.2 Hz, 1H), 4.56 (d, J=9.2 Hz, 1H), 4.45-4.40 (m, 1H), 4.30 (s, 1H), 4.01-3.95 (m, 3H), 3.87 (t, J=5.6 Hz, 3H), 3.69-3.51 (m, 3H), 3.03 (t, J=5.6 Hz, 3H), 2.89 (s, 2H), 2.73 (s, 1H), 2.46-2.42 (m, 3H), 2.06-2.00 (m, 1H), 1.89-1.82 (m, 6H), 1.80-1.70 (m, 4H), 1.47 (d, J=6.8 Hz, 1H), 1.38 (d, J=6.8 Hz, 6H), 1.27-1.23 (m, 1H), 1.01 (s, 9H), 0.98-0.93 (m, 10H)

### Step G. Procedure for Preparation of 6-[8-(1,3-benzothiazol-2-ylcarbamoyl)-3,4-dihydro-1H-isoquinolin-2-yl]-3-[3-[3-[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]propoxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[3-[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]propoxy]-2-methyl-phenyl]pyridine-2-carboxylate (120 mg, 99.79 μmol, 1.0 equiv.) in TFA (1 mL) and DCM (1 mL) was stirred at 25° C. for 2 hours. The residue was concentrated, and the crude product was purified by prep-HPLC to give 6-[8-(1,3-benzothiazol-2-ylcarbamoyl)-3,4-dihydro-1H-isoquinolin-2-yl]-3-[3-[3-[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]propoxy]-2-methyl-phenyl]pyridine-2-carboxylic acid (26.1 mg, 96.0% purity) as an off-white solid.

MS (ESI) m/z: 574.0 [M/2+H]⁺.

¹H NMR (400 MHz, DMSO-d₆) δ=13.05-12.68 (m, 1H), 8.98 (s, 1H), 8.42 (d, J=7.6 Hz, 1H), 8.13 (s, 1H), 8.03 (d, J=7.6 Hz, 1H), 7.78 (d, J=8.0 Hz, 1H), 7.62 (d, J=7.6 Hz, 1H), 7.50-7.31 (m, 9H), 7.14-7.04 (m, 1H), 6.97 (d, J=8.8 Hz, 1H), 6.87 (d, J=8.4 Hz, 1H), 6.63 (d, J=7.6 Hz, 1H), 5.11 (d, J=1.6 Hz, 1H), 4.98 (s, 2H), 4.92-4.84 (m, 1H), 4.50 (d, J=10.0 Hz, 1H), 4.43 (t, J=8.0 Hz, 1H), 4.28 (s, 1H), 3.93 (td, J=6.0, 15.6 Hz, 4H), 3.63-3.51 (m, 2H), 3.47-3.38 (m, 2H), 3.02 (t, J=5.6 Hz, 2H), 2.94-2.78 (m, 2H), 2.62-2.52 (m, 2H), 2.45 (s, 3H), 2.19-2.00 (m, 2H), 1.89 (s, 2H), 1.79-1.64 (m, 5H), 1.50-1.32 (m, 6H), 1.27-1.13 (m, 2H), 0.93 (s, 9H)

### Example 26. Preparation of Compound 125a

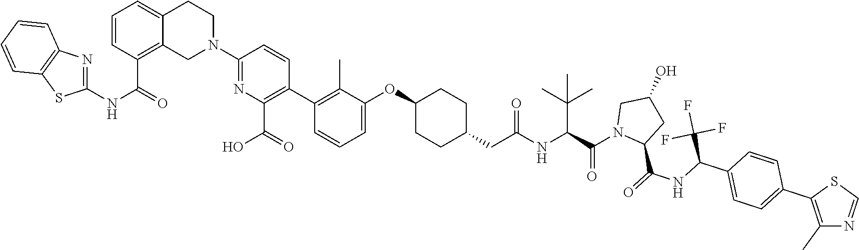 [SMILES: Cc1ncsc1-c1ccc([C@@H](NC(=O)[C@@H]2C[C@@H](O)CN2C(=O)[C@@H](NC(=O)C[C@H]2CC[C@H](Oc3cccc(-c4ccc(N5CCc6cccc(C(=O)Nc7nc8ccccc8s7)c6C5)nc4C(=O)O)c3C)CC2)C(C)(C)C)C(F)(F)F)cc1]

### 6-(8-(benzo[d]thiazol-2-ylcarbamoyl)-3,4-dihydroisoquinolin-2 (1H)-yl)-3-(3-(((1S,4r)-4-(2-(((S)-1-((2S,4R)-4-hydroxy-2-(((R)-2,2,2-trifluoro-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2 (1H)-yl)-3-(3-(((1S,4r)-4-(2-(((S)-1-((2S,4R)-4-hydroxy-2-(((R)-2,2,2-trifluoro-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)oxy)-2-methylphenyl)picolinate

To a solution of 2-((1r,4r)-4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)cyclohexyl)acetic acid (80 mg, 109.16 μmol, 1.0 equiv.) in pyridine (1 mL) was added EDCI (27.20 mg, 141.90 μmol, 1.3 equiv.) and (2S,4R)-1-((S)-2-amino-3,3-dimethylbutanoyl)-4-hydroxy-N—((R)-2,2,2-trifluoro-1-(4-(4-methylthiazol-5-yl)phenyl)ethyl)pyrrolidine-2-carboxamide (54.42 mg, 109.16 μmol, 1.0 equiv.). The mixture was stirred at 40° C. for 1 hour. The reaction mixture was concentrated under reduced pressure to remove solvent. The residue was purified by column chromatography (SiO₂, petroleum ether/ethyl acetate=4/1 to DCM:MeOH=20:1) to give tert-butyl 6-(8-(benzo[d]thiazol-2-ylcarbamoyl)-3,4-dihydroisoquinolin-2 (1H)-yl)-3-(3-(((1S,4r)-4-(2-(((S)-1-((2S,4R)-4-hydroxy-2-(((R)-2,2,2-trifluoro-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)oxy)-2-methylphenyl)picolinate (100 mg, 82.4 μmol, 75.5% yield) as a yellow oil.

MS (ESI) m/z: 1214.2 [M+H]⁺.

### Step B. Procedure for Preparation of 6-(8-(benzo[d]thiazol-2-ylcarbamoyl)-3,4-dihydroisoquinolin-2 (1H)-yl)-3-(3-(((1S,4r)-4-(2-(((S)-1-((2S,4R)-4-hydroxy-2-(((R)-2,2,2-trifluoro-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2 (1H)-yl)-3-(3-(((1S,4r)-4-(2-(((S)-1-((2S,4R)-4-hydroxy-2-(((R)-2,2,2-trifluoro-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)oxy)-2-methylphenyl)picolinate (100 mg, 82.41 μmol, 1.0 equiv.) in DCM (1 mL) was added TFA (9.40 mg, 82.41 μmol, 6.10 μL, 1.0 equiv.). The mixture was stirred at 40° C. for 12 hours. The reaction mixture was concentrated under reduced pressure to remove solvent. The residue was purified by prep-HPLC to give 6-(8-(benzo[d]thiazol-2-ylcarbamoyl)-3,4-dihydroisoquinolin-2 (1H)-yl)-3-(3-(((1S,4r)-4-(2-(((S)-1-((2S,4R)-4-hydroxy-2-(((R)-2,2,2-trifluoro-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)oxy)-2-methylphenyl)picolinic acid (7.1 mg, 5.8 μmol, 7.1% yield) as a yellow solid.

MS (ESI) m/z: 1157.3 [M+H]⁺.

¹H NMR (400 MHz, DMSO-d6) δ=9.30 (d, J=10.0 Hz, 1H), 9.03 (s, 1H), 7.97 (d, J=8.4 Hz, 1H), 7.87 (d, J=9.6 Hz, 1H), 7.75 (d, J=8.4 Hz, 1H), 7.65 (d, J=8.0 Hz, 3H), 7.55 (d, J=8.4 Hz, 2H), 7.45-7.38 (m, 2H), 7.36-7.27 (m, 3H), 7.06-7.00 (m, 1H), 6.88 (d, J=8.4 Hz, 1H), 6.80-6.74 (m, 1H), 6.70-6.66 (m, 1H), 5.89-5.82 (m, 1H), 5.01-4.96 (m, 2H), 4.60-4.52 (m, 2H), 4.29 (d, J=2.4 Hz, 1H), 4.19-4.12 (m, 1H), 3.90 (t, J=5.6 Hz, 2H), 3.64 (s, 2H), 3.00 (d, J=6.0 Hz, 2H), 2.48 (s, 3H), 2.10-2.02 (m, 4H), 1.89 (s, 3H), 1.74-1.71 (m, 2H), 1.32 (d, J=14.4 Hz, 2H), 1.26-1.23 (m, 4H), 1.14-1.09 (m, 2H), 0.95 (s, 9H)

### Example 27. Preparation of Compound 126a

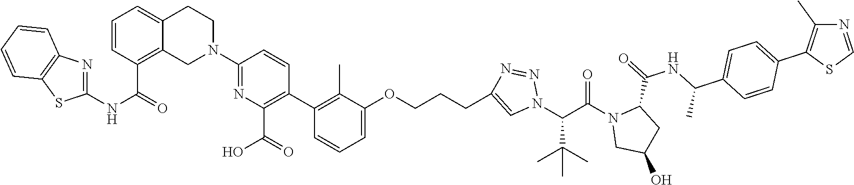 [SMILES: Cc1ncsc1-c1ccc([C@H](C)NC(=O)[C@@H]2C[C@@H](O)CN2C(=O)[C@@H](n2cc(CCCOc3cccc(-c4ccc(N5CCc6cccc(C(=O)Nc7nc8ccccc8s7)c6C5)nc4C(=O)O)c3C)nn2)C(C)(C)C)cc1]

### 6-[8-(1,3-benzothiazol-2-ylcarbamoyl)-3,4-dihydro-1H-isoquinolin-2-yl]-3-[3-[5-[1-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]triazol-4-yl]pentoxy]-2-methyl-phenyl]pyridine-2-carboxylic acid

### Step A. Procedure for Preparation of 1-bromo-3-hept-6-ynoxy-2-methyl-benzene

A mixture of 3-bromo-2-methyl-phenol (1 g, 5.35 mmol, 1 equiv.), 7-bromohept-1-yne (936.01 mg, 5.35 mmol, 1 equiv.), and K₂CO₃ (2.22 g, 16.04 mmol, 3 equiv.) in MeCN (10 mL) was degassed and purged with N₂ three times. The mixture was then stirred at 60° C. for 16 hours under N₂ atmosphere. The reaction mixture was filtered and concentrated under reduced pressure to give 1-bromo-3-hept-6-ynoxy-2-methyl-benzene (1.5 g, crude) as a brown oil.

MS (ESI) m/z: 280.2 [M+H]⁺

### Step B. Procedure for Preparation of (2S,4R)-1-[(2S)-2-azido-3,3-dimethyl-butanoyl]-4-hydroxy-N-[(1S)-1-[4-(4-methylthiazol-5-yl)phenyl]ethyl]pyrrolidine-2-carboxamide

To a solution of (2S,4R)-1-[(2S)-2-amino-3,3-dimethyl-butanoyl]-4-hydroxy-N—[(S)-1-[4-(4-methylthiazol-5-yl)phenyl]ethyl]pyrrolidine-2-carboxamide (1.5 g, 3.37 mmol, 1 equiv.) and N-diazoimidazole-1-sulfonamide (876.30 mg, 5.06 mmol, 1.5 equiv.) in MeOH (15 mL) was added K₂CO₃ (1.63 g, 11.81 mmol, 3.5 equiv.) and CuSO₄ (807.76 mg, 5.06 mmol, 776.69 μL, 1.5 equiv.). The mixture was stirred at 40° C. for 24 hours. (2S,4R)-1-[(2S)-2-azido-3,3-dimethyl-butanoyl]-4-hydroxy-N-[(1S)-1-[4-(4-methylthiazol-5-yl)phenyl]ethyl]pyrrolidine-2-carboxamide (1.5 g, crude) was obtained as a blue liquid, which was used in next step directly.

MS (ESI) m/z: 471.2 [M+H]⁺

### Step C. Procedure for Preparation of (2S,4R)-1-[(2S)-2-[4-[5-(3-bromo-2-methyl-phenoxy)pentyl]triazol-1-yl]-3,3-dimethyl-butanoyl]-4-hydroxy-N-[(1S)-1-[4-(4-methylthiazol-5-yl)phenyl]ethyl]pyrrolidine-2-carboxamide

To a solution of 1-bromo-3-hept-6-ynoxy-2-methyl-benzene (500 mg, 1.78 mmol, 1 equiv.) and (2S,4R)-1-[(2S)-2-azido-3,3-dimethyl-butanoyl]-4-hydroxy-N-[(1S)-1-[4-(4-methylthiazol-5-yl)phenyl]ethyl]pyrrolidine-2-carboxamide (920.46 mg, 1.96 mmol, 1.1 equiv.) in t-BuOH (5 mL) and water (5 mL) was added CuSO₄ (425.72 mg, 2.67 mmol, 409.34 μL, 1.5 equiv.) and sodium;(2R)-2-[(1S)-1,2-dihydroxyethyl]-4-hydroxy-5-oxo-2H-furan-3-olate (1.41 g, 7.11 mmol, 4 equiv.). The mixture was stirred at 25° C. for 18 hours. The reaction mixture was filtered, and the filtrate was concentrated under reduced pressure to give a residue which was purified by reverse-phase HPLC (0.1% NH₃H₂O) to give (2S,4R)-1-[(2S)-2-[4-[5-(3-bromo-2-methyl-phenoxy)pentyl]triazol-1-yl]-3,3-dimethyl-butanoyl]-4-hydroxy-N-[(1S)-1-[4-(4-methylthiazol-5-yl)phenyl]ethyl]pyrrolidine-2-carboxamide (130 mg, 172.9 μmol, 9.7% yield) as a yellow oil.

MS (ESI) m/z: 753.3 [M+H]⁺

### Step D. Procedure for Preparation of tert-butyl 6-[8-(1,3-benzothiazol-2-ylcarbamoyl)-3,4-dihydro-1H-isoquinolin-2-yl]-3-[3-[5-[1-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]triazol-4-yl]pentoxy]-2-methyl-phenyl]pyridine-2-carboxylate

(2S,4R)-1-[(2S)-2-[4-[5-(3-bromo-2-methyl-phenoxy)pentyl]triazol-1-yl]-3,3-dimethyl-butanoyl]-4-hydroxy-N-[(1S)-1-[4-(4-methylthiazol-5-yl)phenyl]ethyl]pyrrolidine-2-carboxamide (130 mg, 172.92 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (127.11 mg, 207.51 μmol, 1.2 equiv.), Ad₂nBuP Pd G₃ (cataCXium® A Pd G₃) (12.59 mg, 17.29 μmol, 0.1 equiv.), and KF (1.5 M, 345.85 μL, 3 equiv.) were taken up into a microwave tube in dioxane (1 mL). The sealed tube was heated at 100° C. for 60 min under microwave. The reaction mixture was then concentrated under reduced pressure to give a residue. The residue was purified by prep-TLC (SiO₂, dichloromethane:methanol=10:1) to give tert-butyl 6-[8-(1,3-benzothiazol-2-ylcarbamoyl)-3,4-dihydro-1H-isoquinolin-2-yl]-3-[3-[5-[1-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]triazol-4-yl]pentoxy]-2-methyl-phenyl]pyridine-2-carboxylate (140 mg, 120.9 μmol, 69.9% yield) as a white solid.

MS (ESI) m/z: 1157.3 [M+H]⁺

### Step E. Procedure for Preparation of 6-[8-(1,3-benzothiazol-2-ylcarbamoyl)-3,4-dihydro-1H-isoquinolin-2-yl]-3-[3-[5-[1-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]triazol-4-yl]pentoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[5-[1-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]triazol-4-yl]pentoxy]-2-methyl-phenyl]pyridine-2-carboxylate (70 mg, 60.48 μmol, 1 equiv.) in DCM (0.5 mL) was added TFA (1.08 g, 9.45 mmol, 699.98 μL, 156.33 equiv.). The mixture was stirred at 25° C. for 12 hours. The reaction mixture was filtered and concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[5-[1-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]triazol-4-yl]pentoxy]-2-methyl-phenyl]pyridine-2-carboxylic acid (42.0 mg, 36.4 μmol, 60.2% yield) as a yellow solid.

MS (ESI) m/z: 1101.5 [M+H]⁺

¹H NMR (400 MHz, DMSO-d₆) δ=13.09-12.27 (m, 2H), 8.98 (s, 1H), 8.51 (d, J=7.6 Hz, 1H), 8.06-8.00 (m, 2H), 7.78 (d, J=8.0 Hz, 1H), 7.62 (d, J=7.2 Hz, 1H), 7.49-7.41 (m, 5H), 7.39-7.30 (m, 4H), 7.12-7.05 (m, 1H), 6.96 (d, J=8.8 Hz, 1H), 6.87 (d, J=8.4 Hz, 1H), 6.62 (d, J=7.6 Hz, 1H), 5.42 (s, 1H), 4.98 (s, 2H), 4.94-4.88 (m, 1H), 4.46-4.37 (m, 1H), 4.28 (s, 1H), 3.99-3.88 (m, 4H), 3.71-3.64 (m, 1H), 3.59 (d, J=10.4 Hz, 1H), 3.26 (s, 2H), 3.02 (t, J=5.6 Hz, 2H), 2.69-2.63 (m, 2H), 2.45 (s, 3H), 2.21 (s, 1H), 2.10-2.02 (m, 1H), 1.88 (s, 3H), 1.79-1.75 (m, 2H), 1.71-1.66 (m, 2H), 1.52-1.47 (m, 2H), 1.38 (d, J=7.2 Hz, 2H), 0.95 (s, 9H).

### Example 28. Preparation of Compound 127a

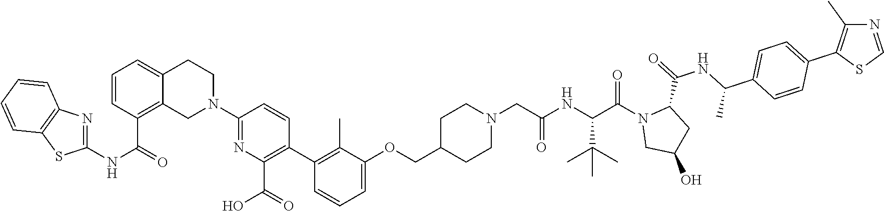 [SMILES: Cc1ncsc1-c1ccc([C@H](C)NC(=O)[C@@H]2C[C@@H](O)CN2C(=O)[C@@H](NC(=O)CN2CCC(COc3cccc(-c4ccc(N5CCc6cccc(C(=O)Nc7nc8ccccc8s7)c6C5)nc4C(=O)O)c3C)CC2)C(C)(C)C)cc1]

### 6-[8-(1,3-benzothiazol-2-ylcarbamoyl)-3,4-dihydro-1H-isoquinolin-2-yl]-3-[3-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]methoxy]-2-methyl-phenyl]pyridine-2-carboxylic acid

### Step A. Procedure for Preparation of tert-butyl 4-[(3-bromo-2-methyl-phenoxy)methyl]piperidine-1-carboxylate

A mixture of tert-butyl 4-(hydroxymethyl)piperidine-1-carboxylate (2.7 g, 12.54 mmol, 1.0 equiv.), 3-bromo-2-methyl-phenol (2.35 g, 12.54 mmol, 1.0 equiv.), and 2-(tributyl-λ5-phosphanylidene)acetonitrile (4.54 g, 18.81 mmol, 1.5 equiv.) in toluene (30 mL) was degassed and purged with N₂ three times. The mixture was then stirred at 120° C. for 16 hours under N₂ atmosphere. The reaction mixture was concentrated under reduced pressure to remove solvent. The reaction mixture was mixed with EtOAc (50 mL×3), dried over sodium sulfate, filtered, and concentrated under reduced pressure to give a residue. The residue was purified by flash silica gel chromatography (eluent of 0˜15% ethyl acetate/petroleum ether) to give tert-butyl 4-[(3-bromo-2-methyl-phenoxy)methyl]piperidine-1-carboxylate (3.7 g, 5.3 mmol, 42.6% yield, 55.5% purity) as a white solid.

MS (ESI) m/z: 330.1 [M−56+H]⁺ (⁸⁰Br)

### Step B. Procedure for Preparation of 4-[(3-bromo-2-methyl-phenoxy)methyl]piperidine

To a solution of tert-butyl 4-[(3-bromo-2-methyl-phenoxy)methyl]piperidine-1-carboxylate (3 g, 7.81 mmol, 1.0 equiv.) in HCl/dioxane (15 mL) and dioxane (10 mL). The mixture was stirred at 25° C. for 4 hours. The mixture was filtered to give a filter cake. The crude product was triturated with DCM (20 mL) at 25° C. for 10 min to give 4-[(3-bromo-2-methyl-phenoxy)methyl]piperidine (1.5 g, 5.2 mmol, 66.9% yield) as a white solid.

MS (ESI) m/z: 284.1 [M+H]+

### Step C. Procedure for Preparation of ethyl 2-[4-[(3-bromo-2-methyl-phenoxy) methyl]-1-piperidyl]acetate

To a solution of 4-[(3-bromo-2-methyl-phenoxy)methyl]piperidine (1.5 g, 4.68 mmol, 1.0 equiv., HCl) in MeCN (15 mL) was added K₂CO₃ (1.94 g, 14.03 mmol, 3.0 equiv.) and ethyl 2-bromoacetate (781.22 mg, 4.68 mmol, 517.37 μL, 1.0 equiv.). The mixture was stirred at 60° C. for 2 hours. The reaction mixture was concentrated under reduced pressure to remove MeCN (15 mL). The residue was diluted with water (50 mL) and extracted with ethyl acetate (50 mL×3). The combined organic layers were dried over sodium sulfate, filtered, and concentrated under reduced pressure to give ethyl 2-[4-[(3-bromo-2-methyl-phenoxy) methyl]-1-piperidyl]acetate (0.9 g, 2.4 mmol) as a yellow oil, which was carried into the next step without further purification.

### Step D. Procedure for Preparation of tert-butyl 6-(8-(benzo[d]thiazol-2-ylcarbamoyl)-3,4-dihydroisoquinolin-2 (1H)-yl)-3-(3-((1-(2-ethoxy-2-oxoethyl)piperidin-4-yl)methoxy)-2-methylphenyl)picolinate

Ethyl 2-[4-[(3-bromo-2-methyl-phenoxy) methyl]-1-piperidyl]acetate (300 mg, 810.20 μmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (595.54 mg, 972.24 μmol, 1.2 equiv.), Ad₂nBuPPd G₃ (29.50 mg, 40.51 μmol, 0.05 equiv.), and KF (141.21 mg, 2.43 mmol, 56.94 μL, 3.0 equiv.) were taken up into a microwave tube in dioxane (5 mL) and water (1 mL). The sealed tube was heated at 100° C. for 2 hours under microwave. The aqueous layer was extracted with ethyl acetate (50 mL×3). The combined organic layers were washed with brine (50 mL), dried over sodium sulfate, concentrated in vacuo to give the crude product as yellow solid. The residue was purified by flash silica gel chromatography (eluent of 0˜50% ethyl acetate/petroleum ether) to give tert-butyl 6-(8-(benzo[d]thiazol-2-ylcarbamoyl)-3,4-dihydroisoquinolin-2 (1H)-yl)-3-(3-((1-(2-ethoxy-2-oxoethyl)piperidin-4-yl)methoxy)-2-methylphenyl)picolinate (340 mg, 438.1 μmol, 54.0% yield) as a yellow solid.

### Step E. Procedure for Preparation of 2-[4-[[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]methyl]-1-piperidyl]acetic acid

To a solution of tert-butyl 6-(8-(benzo[d]thiazol-2-ylcarbamoyl)-3,4-dihydroisoquinolin-2 (1H)-yl)-3-(3-((1-(2-ethoxy-2-oxoethyl)piperidin-4-yl)methoxy)-2-methylphenyl)picolinate (200 mg, 257.75 μmol, 1.0 equiv.) in MeOH (5 mL) was added LiOH·H₂O (43.26 mg, 1.03 mmol, 4.0 equiv.) at 25° C. for 2 hours. The reaction mixture was concentrated under reduced pressure to remove solvent. The pH of the residue was adjusted to 4-5 by HCl (1 N), and then the mixture was filtered to give 2-[4-[[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]methyl]-1-piperidyl]acetic acid (135 mg, 158.8 μmol, 61.6% yield, 88% purity) as a yellow solid.

### Step F. Procedure for Preparation of tert-butyl 6-[8-(1,3-benzothiazol-2-ylcarbamoyl)-3,4-dihydro-1H-isoquinolin-2-yl]-3-[3-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]methoxy]-2-methyl-phenyl]pyridine-2-carboxylate

To a solution of 2-[4-[[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]methyl]-1-piperidyl]acetic acid (100 mg, 133.71 μmol, 1.0 equiv.) and (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (59.44 mg, 133.71 μmol, 1.2 equiv.) in DMF (5 mL) was added DIEA (51.84 mg, 401.12 μmol, 69.87 μL, 3.0 equiv.) and HATU (61.01 mg, 160.45 μmol, 1.2 equiv.). The mixture was stirred at 40° C. for 1 hour. The residue was diluted with water (50 mL) and extracted with EtOAc (50 mL×3). The combined organic layers were dried over sodium sulfate, filtered, and concentrated under reduced pressure to give a residue. The residue was purified by prep-HPLC to give tert-butyl 6-[8-(1,3-benzothiazol-2-ylcarbamoyl)-3,4-dihydro-1H-isoquinolin-2-yl]-3-[3-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]methoxy]-2-methyl-phenyl]pyridine-2-carboxylate (106 mg, 72.2 μmol, 54.0% yield, 80.0% purity) as a yellow oil.

MS (ESI) m/z: 588.2 [M/2+1]⁺

### Step G. Procedure for Preparation of 6-[8-(1,3-benzothiazol-2-ylcarbamoyl)-3,4-dihydro-1H-isoquinolin-2-yl]-3-[3-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]methoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]methoxy]-2-methyl-phenyl]pyridine-2-carboxylate (101 mg, 86.00 μmol, 1.0 equiv.) in TFA (1 mL) and DCM (1 mL) was stirred at 25° C. for 16 hours. The reaction mixture was concentrated under reduced pressure to remove solvent. The residue was purified by prep-HPLC to give 6-[8-(1,3-benzothiazol-2-ylcarbamoyl)-3,4-dihydro-1H-isoquinolin-2-yl]-3-[3-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]methoxy]-2-methyl-phenyl]pyridine-2-carboxylic acid (39.9 mg, 35.3 μmol, 41.1% yield, 99.0% purity) as a yellow solid.

MS (ESI) m/z: 560.1 [M/2+H]⁺

¹HNMR (400 MHz, DMSO-d₆) δ=8.98 (s, 1H), 8.42 (d, J=7.6 Hz, 1H), 8.14 (s, 1H), 8.03 (d, J=8.0 Hz, 1H), 7.79 (d, J=8.4 Hz, 2H), 7.64-7.62 (m, 1H), 7.49-7.45 (m, 6H), 7.37-7.34 (m, 3H), 7.10 (t, J=7.6 Hz, 1H), 6.97 (d, J=8.8 Hz, 1H), 6.88 (d, J=8.4 Hz, 1H), 6.64 (d, J=7.6 Hz, 1H), 5.11 (d, J=3.2 Hz, 1H), 4.98 (s, 2H), 4.86-4.91 (m, 1H), 4.54-4.39 (m, 2H), 4.33-4.24 (m, 1H), 3.94-3.90 (m, 2H), 3.87 (d, J=4.8 Hz, 2H), 3.60-3.57 (m, 2H), 3.03 (d, J=5.6 Hz, 2H), 2.91-2.87 (m, 2H), 2.45 (s, 3H), 2.27-2.14 (m, 3H), 1.92 (s, 3H), 1.87-1.74 (m, 5H), 1.50-1.44 (m, 2H), 1.35 (d, J=7.2 Hz, 3H), 0.94 (s, 9H)

### Example 29. Preparation of Compound 128a

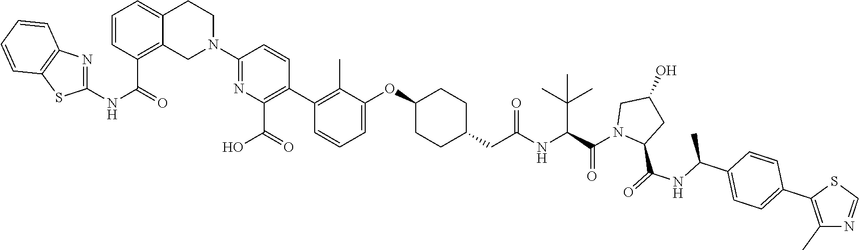 [SMILES: Cc1ncsc1-c1ccc([C@H](C)NC(=O)[C@@H]2C[C@@H](O)CN2C(=O)[C@@H](NC(=O)C[C@H]2CC[C@H](Oc3cccc(-c4ccc(N5CCc6cccc(C(=O)Nc7nc8ccccc8s7)c6C5)nc4C(=O)O)c3C)CC2)C(C)(C)C)cc1]

### 6-(8-(benzo[d]thiazol-2-ylcarbamoyl)-3,4-dihydroisoquinolin-2 (1H)-yl)-3-(3-(((1S,4r)-4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethylbutan-2-yl)amino)-2-oxoethyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2 (1H)-yl)-3-(3-(((1r,4r)-4-(2-methoxy-2-oxoethyl)cyclohexyl)oxy)-2-methylphenyl)picolinate

A mixture of tert-butyl 6-(8-(benzo[d]thiazol-2-ylcarbamoyl)-3,4-dihydroisoquinolin-2 (1H)-yl)-3-(4,4,5,5-tetramethyl-1,3,2-dioxaborolan-2-yl)picolinate (394.91 mg, 644.71 μmol, 1.1 equiv.), methyl 2-((1r,4r)-4-(3-bromo-2-methylphenoxy)cyclohexyl)acetate (200 mg, 586.10 μmol, 1 equiv.), KF (1.5 M, 1.17 mL, 3 equiv.), and [2-(2-aminophenyl)phenyl]palladium(1+);bis(1-adamantyl)-butyl-phosphane;methanesulfonate (42.68 mg, 58.61 μmol, 0.1 equiv.) in dioxane (4 mL) was degassed and purged with N₂ three times. The mixture was then stirred at 100° C. for 1 hour under N₂ atmosphere. The reaction mixture was concentrated under reduced pressure to give a residue. The residue was purified by flash silica gel chromatography (eluent of 0˜25% ethyl acetate/petroleum ether) to give tert-butyl 6-(8-(benzo[d]thiazol-2-ylcarbamoyl)-3,4-dihydroisoquinolin-2 (1H)-yl)-3-(3-(((1r,4r)-4-(2-methoxy-2-oxoethyl)cyclohexyl)oxy)-2-methylphenyl)picolinate (330 mg, 441.8 μmol, 75.3% yield) as a yellow solid.

MS (ESI) m/z: 747.2 [½M+H]⁺

### Step B. Procedure for Preparation of 2-((1r,4r)-4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)cyclohexyl)acetic acid

To a solution of tert-butyl 6-(8-(benzo[d]thiazol-2-ylcarbamoyl)-3,4-dihydroisoquinolin-2 (1H)-yl)-3-(3-(((1r,4r)-4-(2-methoxy-2-oxoethyl)cyclohexyl)oxy)-2-methylphenyl)picolinate (320 mg, 428.43 μmol, 1 equiv.) in THF (3 mL) was added LiOH·H₂O (53.93 mg, 1.29 mmol, 3 equiv.) and water (1 mL). The mixture was stirred at 40° C. for 3 hours. The reaction mixture was concentrated under reduced pressure to remove solvent. To the residue was added water (5 mL), and the pH of mixture was adjusted to 4 with 1M HCl. The resulting mixture was filtered and concentrated under reduced pressure to give 2-((1r,4r)-4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)cyclohexyl)acetic acid (290 mg, 395.7 μmol, 92.3% yield) as a yellow solid.

MS (ESI) m/z: 733.6 [½M+H]⁺

### Step C. Procedure for Preparation of tert-butyl 6-(8-(benzo[d]thiazol-2-ylcarbamoyl)-3,4-dihydroisoquinolin-2 (1H)-yl)-3-(3-(((1S,4r)-4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethylbutan-2-yl)amino)-2-oxoethyl)cyclohexyl)oxy)-2-methylphenyl)picolinate

To a solution of 2-((1r,4r)-4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)cyclohexyl)acetic acid (100 mg, 136.45 μmol, 1 equiv.) in DMF (1 mL) was added HATU (67.45 mg, 177.38 μmol, 1.3 equiv.), DIEA (52.90 mg, 409.34 μmol, 71.30 μL, 3 equiv.), and (2S,4R)-1-((S)-2-amino-3,3-dimethylbutyl)-4-hydroxy-N—((S)-1-(4-(4-methylthiazol-5-yl)phenyl)ethyl)pyrrolidine-2-carboxamide (76.48 mg, 163.74 μmol, 1.2 equiv.). The mixture was stirred at 25° C. for 16 hours. The reaction mixture was treated with water (5 mL), filtered, and concentrated under reduced pressure to give tert-butyl 6-(8-(benzo[d]thiazol-2-ylcarbamoyl)-3,4-dihydroisoquinolin-2 (1H)-yl)-3-(3-(((1S,4r)-4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethylbutan-2-yl)amino)-2-oxoethyl)cyclohexyl)oxy)-2-methylphenyl)picolinate (120 mg, 104.7 μmol, 76.8% yield) as a yellow solid.

MS (ESI) m/z: 573.7 [M/2+H]⁺

### Step D. Procedure for Preparation of 6-(8-(benzo[d]thiazol-2-ylcarbamoyl)-3,4-dihydroisoquinolin-2 (1H)-yl)-3-(3-(((1S,4r)-4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethylbutan-2-yl)amino)-2-oxoethyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2 (1H)-yl)-3-(3-(((1S,4r)-4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethylbutan-2-yl)amino)-2-oxoethyl)cyclohexyl)oxy)-2-methylphenyl)picolinate (120 mg, 104.76 μmol, 1 equiv.) in DCM (1 mL) was added TFA (1.54 g, 13.51 mmol, 1 mL, 128.92 equiv.). The mixture was stirred at 25° C. for 16 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2 (1H)-yl)-3-(3-(((1S,4r)-4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethylbutan-2-yl)amino)-2-oxoethyl)cyclohexyl)oxy)-2-methylphenyl)picolinic acid (30.05 mg, 27.2 μmol, 26.0% yield, 98.7% purity) as an off-white solid.

MS (ESI) m/z: 545.7 [½M+H]⁺

¹H NMR (400 MHz, DMSO-d₆) δ=8.93 (s, 1H), 8.00 (d, J=8.0 Hz, 1H), 7.78 (d, J=8.0 Hz, 1H), 7.63 (d, J=7.6 Hz, 1H), 7.50-7.31 (m, 11H), 7.09-7.04 (m, 1H), 6.98-6.89 (m, 2H), 6.64 (d, J=7.2 Hz, 1H), 5.00 (s, 3H), 4.31-4.13 (m, 2H), 3.93 (t, J=6.0 Hz, 3H), 3.04-3.03 (m, 4H), 2.54-2.52 (m, 4H), 2.45 (s, 3H), 2.16-2.06 (m, 5H), 1.89 (s, 3H), 1.85-1.81 (m, 2H), 1.49 (d, J=7.2 Hz, 4H), 1.45-1.36 (m, 2H), 1.28-1.11 (m, 3H), 0.83 (s, 9H)

### Example 30. Preparation of Compound 129a

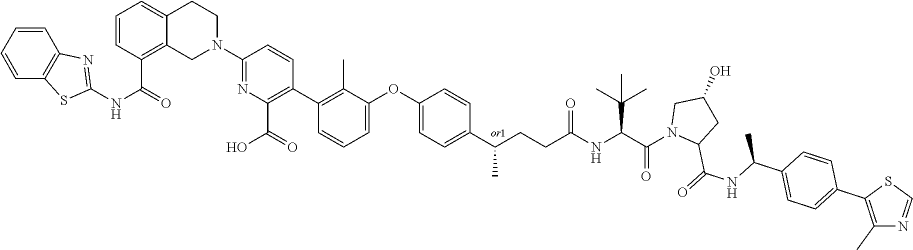 [SMILES: Cc1ncsc1-c1ccc([C@H](C)NC(=O)C2C[C@@H](O)CN2C(=O)[C@@H](NC(=O)CC[C@H](C)c2ccc(Oc3cccc(-c4ccc(N5CCc6cccc(C(=O)Nc7nc8ccccc8s7)c6C5)nc4C(=O)O)c3C)cc2)C(C)(C)C)cc1]

### 6-(8-(benzo[d]thiazol-2-ylcarbamoyl)-3,4-dihydroisoquinolin-2 (1H)-yl)-3-(3-(4-((S)-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)-2-methylphenyl)picolinic acid [6-(8-(benzo[d]thiazol-2-ylcarbamoyl)-3,4-dihydroisoquinolin-2 (1H)-yl)-3-(3-(4-((S*)-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)-2-methylphenyl)picolinic acid]

### Step A. Procedure for Preparation of 1-(4-(3-bromo-2-methylphenoxy)phenyl)ethan-1-one

A mixture of 1,3-dibromo-2-methylbenzene (18.36 g, 73.45 mmol, 2.0 equiv.), 1-(4-hydroxyphenyl)ethan-1-one (5 g, 36.72 mmol, 1.0 equiv.), Cs₂CO₃ (14.36 g, 44.07 mmol, 1.2 equiv.), and 2,2,6,6-tetramethylheptane-3,5-dione (1.69 g, 9.18 mmol, 1.89 mL, 0.25 equiv.) in NMP (50 mL) was degassed and purged with N₂ three times. CuI (3.50 g, 18.36 mmol, 0.5 equiv.) was added to the mixture, which was stirred at 120° C. for 12 hours under N₂ atmosphere. The reaction mixture was diluted with water (100 mL) and extracted with EtOAc (100 mL×3). The combined organic layers were washed with NaHCO₃ (150 mL×3), dried over anhydrous sodium sulfate, filtered, and concentrated under reduced pressure to give a residue. The residue was purified by flash silica gel chromatography (eluent of 0˜3% ethyl acetate/petroleum ether) to give 1-(4-(3-bromo-2-methylphenoxy)phenyl)ethan-1-one (8 g, 26.2 mmol, 71.3% yield) as black-green solid.

¹H NMR (400 MHz, DMSO-d₆) δ=7.96 (d, J=8.8 Hz, 2H), 7.53 (d, J=8.0 Hz, 1H), 7.23 (t, J=8.0 Hz, 1H), 7.07 (d, J=8.0 Hz, 1H), 6.96 (d, J=8.4 Hz, 2H), 2.52 (s, 3H), 2.20 (s, 3H)

### Step B. Procedure for Preparation of (E)-1-(4-(5-(benzyloxy) pent-2-en-2-yl)phenoxy)-3-bromo-2-methylbenzene

To a mixture of (3-(benzyloxy)propyl)triphenylphosphonium (9.66 g, 19.66 mmol, 1.5 equiv.) was added NaH (1.31 g, 32.77 mmol, 60% purity, 2.5 equiv.) in THF (30 mL) at −10° C. under N₂ atmosphere for 3 hours. Then 1-(4-(3-bromo-2-methylphenoxy)phenyl)ethan-1-one (4 g, 13.11 mmol, 1.0 equiv.) was added to the mixture at 0° C. under N₂ atmosphere. The mixture was stirred at 40° C. for 16 hours under N₂ atmosphere. The reaction mixture was quenched by addition water (50 mL) at 25° C. and extracted with EtOAc (60 mL×3). The combined organic layers were washed with brine (60 mL×3), dried over anhydrous sodium sulfate filtered, and concentrated under reduced pressure to give a residue. The residue was purified by flash silica gel chromatography to give (E)-1-(4-(5-(benzyloxy) pent-2-en-2-yl)phenoxy)-3-bromo-2-methylbenzene (4.9 g, 11.2 mmol, 85.4% yield) as yellow oil.

¹H NMR (400 MHz, DMSO-d₆) δ=7.47-7.42 (m, 1H), 7.40-7.30 (m, 3H), 7.30-7.26 (m, 2H), 7.24-7.19 (m, 2H), 7.19-7.10 (m, 1H), 6.95 (d, J=8.0 Hz, 1H), 6.92-6.85 (m, 2H), 5.49 (td, J=7.2, 1.2 Hz, 1H), 4.42 (s, 2H), 3.43 (t, J=6.8 Hz, 2H), 2.32-2.22 (m, 5H), 1.99 (s, 3H)

### Step C. Procedure for Preparation of 4-[4-(3-bromo-2-methyl-phenoxy)phenyl]pentan-1-ol

A mixture of (E)-1-(4-(5-(benzyloxy) pent-2-en-2-yl)phenoxy)-3-bromo-2-methylbenzene (4 g, 9.15 mmol, 1 equiv.) and PtO₂ (207.68 mg, 914.56 μmol, 0.1 equiv.) in MeOH (40 mL) was degassed and purged with H₂ three times. The mixture was then stirred at 40° C. for 36 hours under H₂ atmosphere. The reaction mixture was filtered and concentrated under reduced pressure to give a residue. The residue was purified by reverse-phase HPLC (with 0.1% HCOOH) to give 4-[4-(3-bromo-2-methyl-phenoxy)phenyl]pentan-1-ol (400 mg, 1.1 mmol, 12.4% yield, 99.8% purity) as colorless oil.

MS (ESI) m/z: 349.2 [M+H]⁺

¹H NMR (400 MHz, DMSO-d₆) δ=7.41 (d, J=8.0 Hz, 1H), 7.22-7.18 (m, 2H), 7.17-7.14 (m, 1H), 6.89-6.84 (m, 3H), 4.32 (t, J=5.1 Hz, 1H), 3.36-3.33 (m, 2H), 2.68-2.62 (m, 1H), 2.27 (s, 3H), 1.55-1.49 (m, 2H), 1.33-1.23 (m, 2H), 1.17 (d, J=6.8 Hz, 3H)

### Step D. Procedure for Preparation of (S)-4-(4-(3-bromo-2-methylphenoxy)phenyl)pentan-1-ol

The stereoisomers of 4-[4-(3-bromo-2-methyl-phenoxy)phenyl]pentan-1-ol were separated by SFC to give (S)-4-(4-(3-bromo-2-methylphenoxy)phenyl)pentan-1-ol (100 mg, 286.3 μmol, 25.0% yield) as a colorless oil and (R)-4-(4-(3-bromo-2-methylphenoxy)phenyl)pentan-1-ol (100 mg, 286.3 μmol, 25.0% yield) as a yellow oil.

### Step E. Procedure for Preparation of (S)-4-(4-(3-bromo-2-methylphenoxy)phenyl)pentanal

A mixture of (R)-4-(4-(3-bromo-2-methylphenoxy)phenyl)pentan-1-ol (100 mg, 286.32 μmol, 1.0 equiv.) and (1,1-diacetoxy-3-oxo-1λ⁵,2-benziodoxol-1-yl) acetate (242.88 mg, 572.64 μmol, 177.28 μL, 2.0 equiv.) in DCM (2 mL) was degassed and purged with N₂ three times, and then the mixture was stirred at 25° C. for 3 hours under N₂ atmosphere. The reaction mixture was diluted with water (20 mL) and extracted with DCM (25 mL×3). The combined organic layers were washed with brine (15 mL×3), dried over anhydrous sodium sulfate, filtered, and concentrated under reduced pressure to give (S)-4-(4-(3-bromo-2-methylphenoxy)phenyl)pentanal (100 mg, crude) as yellow oil.

¹H NMR (400 MHz, DMSO-d₆) δ=9.60-9.58 (m, 1H), 7.41 (dd, J=8.0, 0.8 Hz, 1H), 7.22-7.18 (m, 2H), 7.17-7.13 (m, 1H), 6.89-6.84 (m, 3H), 2.72-2.64 (m, 1H), 2.41-2.29 (m, 2H), 2.27 (s, 3H), 1.83-1.74 (m, 2H), 1.19 (d, J=6.8 Hz, 3H)

### Step F. Procedure for Preparation of (S)-4-(4-(3-bromo-2-methylphenoxy)phenyl)pentanoic acid

A mixture of (S)-4-(4-(3-bromo-2-methylphenoxy)phenyl)pentanal (100 mg, 287.98 μmol, 1.0 equiv.), sodium chlorite (78.14 mg, 863.94 μmol, 3.0 equiv.), sodium dihydrogen phosphate (103.65 mg, 863.94 μmol, 3.0 equiv.), and 2-methylbut-2-ene (121.18 mg, 1.73 mmol, 183.05 μL, 6.0 equiv.) in t-BuOH (4 mL), THE (2 mL), and water (1 mL) was stirred at 25° C. for 16 hours under O₂ atmosphere. The reaction mixture was diluted with water (20 mL) and extracted with EtOAc (25 mL×3). The combined organic layers were washed with brine (15 mL×3), dried over anhydrous sodium sulfate, filtered, and concentrated under reduced pressure to give a residue. The residue was purified by prep-TLC (SiO₂, PE:EA=3:1) to give (S)-4-(4-(3-bromo-2-methylphenoxy)phenyl)pentanoic acid (80 mg, 220.2 μmol, 76.4% yield) as yellow oil.

¹H NMR (400 MHz, DMSO-d₆) δ=12.50-11.08 (m, 1H), 7.41 (d, J=8.0 Hz, 1H), 7.20 (d, J=8.4 Hz, 2H), 7.15 (t, J=8.0 Hz, 1H), 6.90-6.84 (m, 3H), 2.72-2.65 (m, 1H), 2.27 (s, 3H), 2.09-2.00 (m, 2H), 1.78-1.71 (m, 2H), 1.18 (d, J=6.8 Hz, 3H)

### Step G. Procedure for Preparation of (S)-4-(4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)phenyl)pentanoic acid

(S)-4-(4-(3-bromo-2-methylphenoxy)phenyl)pentanoic acid, tert-butyl 6-(8-(benzo[d]thiazol-2-ylcarbamoyl)-3,4-dihydroisoquinolin-2 (1H)-yl)-3-(4,4,5,5-tetramethyl-1,3,2-dioxaborolan-2-yl)picolinate (134.91 mg, 220.24 μmol, 1.0 equiv.), Ad₂nBuP Pd G₃ (cataCXium® A Pd G₃) (16.04 mg, 22.02 μmol, 0.1 equiv.), and KF (1.5 M, 440.47 μL, 3.0 equiv.) were taken up into a microwave tube in dioxane (2 mL). The sealed tube was heated at 100° C. for 1 hour under microwave. The reaction mixture was filtered and concentrated under reduced pressure to give a residue. The residue was purified by prep-TLC (SiO₂, petroleum ether:ethyl acetate=1:1) to give (S)-4-(4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)phenyl)pentanoic acid (50 mg, 65.0 μmol, 29.5% yield) as yellow oil.

MS (ESI) m/z: 769.3 [M+H]⁺

### Step H. Procedure for Preparation of tert-butyl 6-(8-(benzo[d]thiazol-2-ylcarbamoyl)-3,4-dihydroisoquinolin-2 (1H)-yl)-3-(3-(4-((S)-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)-2-methylphenyl)picolinate

A mixture of (S)-4-(4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)phenyl)pentanoic acid (50 mg, 65.03 μmol, 1.0 equiv.), (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (34.69 mg, 78.03 μmol, 1.2 equiv.), and DIEA (25.21 mg, 195.08 μmol, 33.98 μL, 3.0 equiv.) in DMF (1 mL) was degassed and purged with N₂ three times. The mixture was stirred at 25° C. for 5 minutes. After 5 minutes, HATU (29.67 mg, 78.03 μmol, 1.2 equiv.) was added, and the mixture was stirred at 25° C. for 1 hour under N₂ atmosphere. The reaction mixture was slowly added dropwise in water (5 mL), and a yellow solid was precipitated. The yellow solid was tert-butyl 6-(8-(benzo[d]thiazol-2-ylcarbamoyl)-3,4-dihydroisoquinolin-2 (1H)-yl)-3-(3-(4-((S)-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)-2-methylphenyl)picolinate (50 mg, crude) as yellow oil.

MS (ESI) m/z: 1195.4 [M+H]⁺

### Step I. Procedure for Preparation of 6-(8-(benzo[d]thiazol-2-ylcarbamoyl)-3,4-dihydroisoquinolin-2 (1H)-yl)-3-(3-(4-((S)-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)-2-methylphenyl)picolinic acid [6-(8-(benzo[d]thiazol-2-ylcarbamoyl)-3,4-dihydroisoquinolin-2 (1H)-yl)-3-(3-(4-((S*)-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)-2-methylphenyl)picolinic acid]

A mixture of tert-butyl 6-(8-(benzo[d]thiazol-2-ylcarbamoyl)-3,4-dihydroisoquinolin-2 (1i)-yl)-3-(3-(4-((S)-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)-2-methylphenyl)picolinate (50 mg, 41.82 μmol, 1.0 equiv.) in TFA (1 mL) and DCM (5 mL) was degassed and purged with N₂ three times, and then the mixture was stirred at 25° C. for 3 hours under N₂ atmosphere. The reaction mixture was concentrated under reduced pressure to give a residue. The residue was purified by prep-HPLC to give 6-(8-(benzo[d]thiazol-2-ylcarbamoyl)-3,4-dihydroisoquinolin-2 (1H)-yl)-3-(3-(4-((S)-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)-2-methylphenyl)picolinic acid [6-(8-(benzo[d]thiazol-2-ylcarbamoyl)-3,4-dihydroisoquinolin-2 (1H)-yl)-3-(3-(4-((S*)-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)-2-methylphenyl)picolinic acid](17.9 mg, 15.4 μmol, 36.9% yield) as yellow solid.

MS (ESI) m/z: 1139.4 [M+H]⁺

¹H NMR (400 MHz, DMSO-d₆) δ=13.21-11.95 (m, 1H), 8.98 (s, 1H), 8.37 (d, J=8.0 Hz, 1H), 8.03 (d, J=8.0 Hz, 1H), 7.84-7.75 (m, 2H), 7.63 (d, J=7.2 Hz, 1H), 7.52 (d, J=8.4 Hz, 1H), 7.48-7.41 (m, 4H), 7.40-7.33 (m, 4H), 7.19-7.12 (m, 3H), 6.98 (d, J=8.4 Hz, 1H), 6.89 (d, J=7.6 Hz, 1H), 6.86-6.79 (m, 3H), 4.99 (s, 2H), 4.94-4.86 (m, 1H), 4.53-4.46 (m, 1H), 4.42 (t, J=8.0 Hz, 1H), 4.27 (s, 1H), 3.93 (t, J=5.2 Hz, 2H), 3.60 (s, 2H), 3.07-3.01 (m, 2H), 2.45 (s, 3H), 2.15-2.07 (m, 2H), 2.01 (d, J=4.0 Hz, 1H), 1.92 (s, 3H), 1.88-1.59 (m, 4H), 1.37 (d, J=6.8 Hz, 3H), 1.23-1.12 (m, 4H), 0.92 (s, 9H)

### Example 31. Preparation of Compound 129b

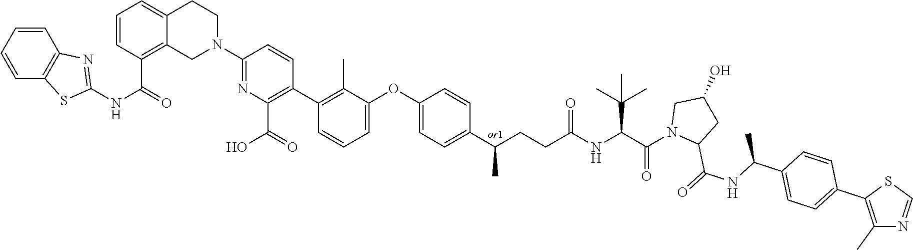 [SMILES: Cc1ncsc1-c1ccc([C@H](C)NC(=O)C2C[C@@H](O)CN2C(=O)[C@@H](NC(=O)CC[C@@H](C)c2ccc(Oc3cccc(-c4ccc(N5CCc6cccc(C(=O)Nc7nc8ccccc8s7)c6C5)nc4C(=O)O)c3C)cc2)C(C)(C)C)cc1]

### 6-(8-(benzo[d]thiazol-2-ylcarbamoyl)-3,4-dihydroisoquinolin-2 (1H)-yl)-3-(3-(4-((R)-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)-2-methylphenyl)picolinic acid [6-(8-(benzo[d]thiazol-2-ylcarbamoyl)-3,4-dihydroisoquinolin-2 (1H)-yl)-3-(3-(4-((R*)-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)-2-methylphenyl)picolinic acid]

### Step A. Procedure for Preparation of (R)-4-(4-(3-bromo-2-methylphenoxy)phenyl)pentanal

A mixture of (R)-4-(4-(3-bromo-2-methylphenoxy)phenyl)pentan-1-ol (100 mg, 286.32 μmol, 1 equiv.) and (1,1-diacetoxy-3-oxo-1λ⁵,2-benziodoxol-1-yl)acetate (242.88 mg, 572.64 μmol, 177.28 μL, 2 equiv.) in DCM (2 mL) was degassed and purged with N₂ three times. The mixture was then stirred at 25° C. for 3 hours under N₂ atmosphere. The reaction mixture was diluted with water (20 mL) and extracted with DCM (25 mL×3). The combined organic layers were washed with brine (15 mL×3), dried over anhydrous sodium sulfate, filtered, and concentrated under reduced pressure to give (R)-4-(4-(3-bromo-2-methylphenoxy)phenyl)pentanal (100 mg, crude) was obtained as a yellow oil.

### Step B. Procedure for Preparation of (R)-4-(4-(3-bromo-2-methylphenoxy)phenyl)pentanoic acid

A mixture of (R)-4-(4-(3-bromo-2-methylphenoxy)phenyl)pentanal (100 mg, 287.98 μmol, 1 equiv.), sodium chlorite (78.14 mg, 863.94 μmol, 3 equiv.), sodium dihydrogen phosphate (103.65 mg, 863.94 μmol, 3 equiv.), and 2-methylbut-2-ene (121.18 mg, 1.73 mmol, 183.05 μL, 6 equiv.) in t-BuOH (4 mL), THE (2 mL) and water (1 mL). The mixture was stirred at 25° C. for 12 hours under O₂ atmosphere. The reaction mixture was diluted with water (20 mL) and extracted with EtOAc (25 mL×3). The combined organic layers were washed with brine (15 mL×3), dried over anhydrous sodium sulfate, filtered, and concentrated under reduced pressure to give a residue. The residue was purified by prep-TLC (SiO₂, PE:EA=3:1) to give (R)-4-(4-(3-bromo-2-methylphenoxy)phenyl)pentanoic acid (90 mg, 247.7 μmol, 86.0% yield) as yellow oil.

### Step C. Procedure for Preparation of (R)-4-(4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)phenyl)pentanoic acid

(R)-4-(4-(3-bromo-2-methylphenoxy)phenyl)pentanoic acid (90 mg, 247.77 μmol, 1 equiv.), tert-butyl 6-(8-(benzo[d]thiazol-2-ylcarbamoyl)-3,4-dihydroisoquinolin-2 (1H)-yl)-3-(4,4,5,5-tetramethyl-1,3,2-dioxaborolan-2-yl)picolinate (182.12 mg, 297.32 μmol, 1.2 equiv.), Ad₂nBuP Pd G3 (cataCXium® A Pd G₃) (18.04 mg, 24.78 μmol, 0.1 equiv.), and KF (1.5 M, 495.53 μL, 3 equiv.) were taken up into a microwave tube in dioxane (2 mL). The sealed tube was heated at 100° C. for 1 hour under microwave. The reaction mixture was filtered and concentrated under reduced pressure to give a residue. The residue was purified by prep-TLC (SiO₂, PE:EA=1:1) to give (R)-4-(4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)phenyl)pentanoic acid (75 mg, 97.5 μmol, 39.3% yield) as yellow oil.

MS (ESI) m/z: 769.1 [M+H]⁺

### Step D. Procedure for Preparation of tert-butyl 6-(8-(benzo[d]thiazol-2-ylcarbamoyl)-3,4-dihydroisoquinolin-2 (1H)-yl)-3-(3-(4-((R)-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)-2-methylphenyl)picolinate

A mixture of (R)-4-(4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)phenyl)pentanoic acid (75 mg, 97.54 μmol, 1 equiv.), (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (52.04 mg, 117.05 μmol, 1.2 equiv.), and DIEA (37.82 mg, 292.62 μmol, 50.97 μL, 3 equiv.) in DMF (1 mL) was degassed and purged with N₂ three times. The mixture was then stirred at 25° C. for 5 minutes. After 5 minutes, HATU (44.51 mg, 117.05 μmol, 1.2 equiv.) was added, and the mixture was stirred at 25° C. for 1 hour under N₂ atmosphere. The reaction mixture was slowly added dropwise to water (5 mL), and a yellow solid was precipitated. The yellow solid was tert-butyl 6-(8-(benzo[d]thiazol-2-ylcarbamoyl)-3,4-dihydroisoquinolin-2 (1H)-yl)-3-(3-(4-((R)-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)-2-methylphenyl)picolinate (70 mg, crude), which was used to next step without purification.

MS (ESI) m/z: 598.5 [M/2+H]⁺

### Step E. Procedure for Preparation of 6-(8-(benzo[d]thiazol-2-ylcarbamoyl)-3,4-dihydroisoquinolin-2 (1H)-yl)-3-(3-(4-((R)-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)-2-methylphenyl)picolinic acid [6-(8-(benzo[d]thiazol-2-ylcarbamoyl)-3,4-dihydroisoquinolin-2 (1H)-yl)-3-(3-(4-((R*)-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)-2-methylphenyl)picolinic acid]

A mixture of tert-butyl 6-(8-(benzo[d]thiazol-2-ylcarbamoyl)-3,4-dihydroisoquinolin-2 (1i)-yl)-3-(3-(4-((R)-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)-2-methylphenyl)picolinate (70 mg, 58.55 μmol, 1 equiv.) in TFA (1 mL) and DCM (1 mL) was degassed and purged with N₂ three times. The mixture was stirred at 25° C. for 3 hours under N₂ atmosphere. The reaction mixture was concentrated under reduced pressure to give a residue. The residue was purified by prep-HPLC to give 6-(8-(benzo[d]thiazol-2-ylcarbamoyl)-3,4-dihydroisoquinolin-2 (1H)-yl)-3-(3-(4-((R)-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)-2-methylphenyl)picolinic acid [6-(8-(benzo[d]thiazol-2-ylcarbamoyl)-3,4-dihydroisoquinolin-2 (1H)-yl)-3-(3-(4-((R*)-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)-2-methylphenyl)picolinic acid](12.7 mg, 10.8 μmol, 18.4% yield, 96.4% purity) as white solid.

MS (ESI) m/z: 1139.1 [M+H]⁺

¹H NMR (400 MHz, DMSO-d₆) δ=8.99-8.95 (m, 1H), 8.35 (d, J=7.4 Hz, 1H), 8.06-7.96 (m, 1H), 7.78 (d, J=8.0 Hz, 2H), 7.66-7.59 (m, 1H), 7.43 (d, J=8.4 Hz, 5H), 7.39-7.31 (m, 4H), 7.21-7.09 (m, 3H), 6.98-6.87 (m, 2H), 6.83 (d, J=8.4 Hz, 3H), 4.99 (s, 2H), 4.92-4.86 (m, 1H), 4.49 (d, J=9.2 Hz, 1H), 4.40 (t, J=8.0 Hz, 1H), 4.27 (s, 1H), 3.92 (s, 2H), 3.59 (s, 2H), 3.02 (s, 2H), 2.70-2.56 (m, 2H), 2.45 (s, 3H), 2.18-2.10 (m, 1H), 2.05-1.97 (m, 2H), 1.92 (s, 3H), 1.82-1.59 (m, 4H), 1.36 (d, J=6.8 Hz, 3H), 1.17 (d, J=6.8 Hz, 4H), 0.92 (s, 9H)

### Example 32. Preparation of Compound 130a

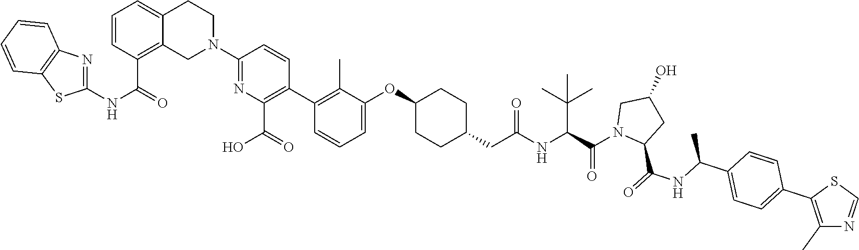 [SMILES: Cc1ncsc1-c1ccc([C@H](C)NC(=O)[C@@H]2C[C@@H](O)CN2C(=O)[C@@H](NC(=O)C[C@H]2CC[C@H](Oc3cccc(-c4ccc(N5CCc6cccc(C(=O)Nc7nc8ccccc8s7)c6C5)nc4C(=O)O)c3C)CC2)C(C)(C)C)cc1]

### 6-(8-(benzo[d]thiazol-2-ylcarbamoyl)-3,4-dihydroisoquinolin-2 (1H)-yl)-3-(3-(((1S,4r)-4-(2-(((S)-1-((2S,4R)-4-hydroxy-2-(((S)-1-(5-(4-methylthiazol-5-yl)pyridin-2-yl)ethyl)carbamoyl)pyrrolidin-1-yl)-3,3-dimethyl-1-oxobutan-2-yl)amino)-2-oxoethyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2 (1H)-yl)-3-(3-(((1S,4r)-4-(2-(((S)-1-((2S,4R)-4-hydroxy-2-(((S)-1-(5-(4-methylthiazol-5-yl)pyridin-2-yl)ethyl)carbamoyl)pyrrolidin-1-yl)-3,3-dimethyl-1-oxobutan-2-yl)amino)-2-oxoethyl)cyclohexyl)oxy)-2-methylphenyl)picolinate

To a solution of 2-((1r,4r)-4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)cyclohexyl)acetic acid (100 mg, 136.45 μmol, 1 equiv.) in DMF (1 mL) was added HATU (67.45 mg, 177.38 μmol, 1.3 equiv.), DIEA (52.90 mg, 409.34 μmol, 71.30 μL, 3 equiv.), and (2S,4R)-1-((S)-2-amino-3,3-dimethylbutanoyl)-4-hydroxy-N—((S)-1-(5-(4-methylthiazol-5-yl)pyridin-2-yl)ethyl)pyrrolidine-2-carboxamide (78.93 mg, 163.74 μmol, 1.2 equiv., HCl). The mixture was stirred at 25° C. for 1 hour. The reaction mixture was treated with water (5 mL), filtered, and concentrated under reduced pressure to give tert-butyl 6-(8-(benzo[d]thiazol-2-ylcarbamoyl)-3,4-dihydroisoquinolin-2 (1H)-yl)-3-(3-(((1S,4r)-4-(2-(((S)-1-((2S,4R)-4-hydroxy-2-(((S)-1-(5-(4-methylthiazol-5-yl)pyridin-2-yl)ethyl)carbamoyl)pyrrolidin-1-yl)-3,3-dimethyl-1-oxobutan-2-yl)amino)-2-oxoethyl)cyclohexyl)oxy)-2-methylphenyl)picolinate (100 mg, 86.1 μmol, 63.1% yield) as a yellow solid.

MS (ESI) m/z: 581.2 [M12+H]⁺

### Step B. Procedure for Preparation of 6-(8-(benzo[d]thiazol-2-ylcarbamoyl)-3,4-dihydroisoquinolin-2 (1H)-yl)-3-(3-(((1S,4r)-4-(2-(((S)-1-((2S,4R)-4-hydroxy-2-(((S)-1-(5-(4-methylthiazol-5-yl)pyridin-2-yl)ethyl)carbamoyl)pyrrolidin-1-yl)-3,3-dimethyl-1-oxobutan-2-yl)amino)-2-oxoethyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2 (1H)-yl)-3-(3-(((1S,4r)-4-(2-(((S)-1-((2S,4R)-4-hydroxy-2-(((S)-1-(5-(4-methylthiazol-5-yl)pyridin-2-yl)ethyl)carbamoyl)pyrrolidin-1-yl)-3,3-dimethyl-1-oxobutan-2-yl)amino)-2-oxoethyl)cyclohexyl)oxy)-2-methylphenyl)picolinate (100 mg, 86.17 μmol, 1 equiv.) in DCM (1 mL) was added TFA (1.27 g, 11.11 mmol, 822.56 μL, 128.92 equiv.). The mixture was stirred at 25° C. for 16 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2 (1H)-yl)-3-(3-(((1S,4r)-4-(2-(((S)-1-((2S,4R)-4-hydroxy-2-(((S)-1-(5-(4-methylthiazol-5-yl)pyridin-2-yl)ethyl)carbamoyl)pyrrolidin-1-yl)-3,3-dimethyl-1-oxobutan-2-yl)amino)-2-oxoethyl)cyclohexyl)oxy)-2-methylphenyl)picolinic acid (14.1 mg, 11.9 μmol, 13.8% yield) as a yellow solid.

MS (ESI) m/z: 553.1 [½M+H]⁺

¹H NMR (400 MHz, DMSO-d₆) δ=12.86 (s, 1H), 12.79-12.34 (m, 1H), 9.07 (s, 1H), 8.63 (d, J=2.0 Hz, 1H), 8.38 (d, J=7.6 Hz, 1H), 8.03 (d, J=8.0 Hz, 1H), 7.92-7.86 (m, 1H), 7.85-7.76 (m, 2H), 7.62 (d, J=8.0 Hz, 1H), 7.49-7.41 (m, 4H), 7.39-7.32 (m, 2H), 7.09-7.04 (m, 1H), 6.99-6.91 (m, 2H), 6.63-6.58 (m, 1H), 4.97 (s, 2H), 4.96-4.91 (m, 1H), 4.52 (d, J=9.6 Hz, 1H), 4.45 (t, J=8.0 Hz, 1H), 4.28 (s, 1H), 4.23-4.13 (m, 1H), 3.91 (t, J=5.6 Hz, 2H), 3.62 (s, 2H), 3.02 (t, J=5.6 Hz, 2H), 2.46 (s, 3H), 2.22-2.16 (m, 1H), 2.09-1.99 (m, 4H), 1.86 (s, 3H), 1.80-1.62 (m, 4H), 1.41 (d, J=7.2 Hz, 3H), 1.37-1.28 (m, 2H), 1.26-1.21 (m, 1H), 1.13-1.04 (m, 2H), 0.96-0.91 (m, 9H)

### Example 33. Preparation of Compound 131a

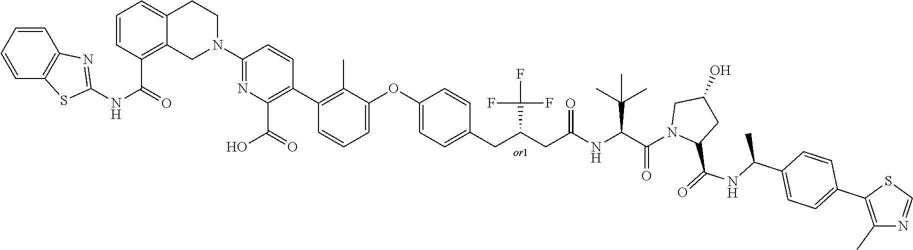

### 6-[8-(1,3-benzothiazol-2-ylcarbamoyl)-3,4-dihydro-1H-isoquinolin-2-yl]-3-[3-[4-[(2S)-4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-4-oxo-2-(trifluoromethyl)butyl]phenoxy]-2-methyl-phenyl]pyridine-2-carboxylic acid [6-(8-(benzo[d]thiazol-2-ylcarbamoyl)-3,4-dihydroisoquinolin-2 (1H)-yl)-3-(3-(4-((S*)-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxo-2-(trifluoromethyl)butyl)phenoxy)-2-methylphenyl)picolinic acid]

### Step A. Procedure for Preparation of 1,1,1-trifluoro-3-(4-methoxyphenyl)propan-2-one

A mixture of methyl 2-(4-methoxyphenyl)acetate (10 g, 55.49 mmol, 8.77 mL, 1 equiv.), trimethyl(trifluoromethyl)silane (11.84 g, 83.24 mmol, 1.5 equiv.), and tetrabutylammonium acetate (836.59 mg, 2.77 mmol, 845.04 μL, 0.05 equiv.) in toluene (100 mL) was degassed and purged with N₂ three times. The mixture was then stirred at 0° C. for 1 hour under N₂ atmosphere. The mixture was filtered and concentrated into a residue. The residue was purified by flash silica gel chromatography (eluent of 0˜20% ethyl acetate/petroleum ether). 1,1,1-Trifluoro-3-(4-methoxyphenyl)propan-2-one (0.7 g, 3.2 mmol, 5.8% yield) was obtained as a colorless oil.

¹H NMR (400 MHz, DMSO-d₆) δ=7.14 (d, J=8.4 Hz, 2H), 6.93-6.89 (m, 2H), 3.96 (s, 2H), 3.82 (s, 3H)

### Step B. Procedure for Preparation of ethyl (Z)-4,4,4-trifluoro-3-[(4-methoxyphenyl)methyl]but-2-enoate

To a solution of ethyl 2-diethoxyphosphorylacetate (863.17 mg, 3.85 mmol, 763.87 μL, 1.2 equiv.) in THF (8 mL) was added LiHMDS (1 M, 3.85 mL, 1.2 equiv.) under −78° C. and N₂ atmosphere. Then the mixture was stirred at −78° C. for 0.5 hour, and a solution of 1,1,1-trifluoro-3-(4-methoxyphenyl)propan-2-one (700 mg, 3.21 mmol, 1 equiv.) in THE (2 mL) was added. The mixture was stirred at 25° C. for 2 hours. The reaction mixture was quenched by the addition of saturated NH₄Cl (10 mL) at 0° C., diluted with EtOAc (10 mL), and extracted with ethyl acetate (10 mL×3). The combined organic layers were washed with water (10 mL), dried over sodium sulfate, filtered, and concentrated under reduced pressure to give a residue. The residue was purified by flash silica gel chromatography (eluent of 0˜10% ethyl acetate/petroleum ether). Ethyl (Z)-4,4,4-trifluoro-3-[(4-methoxyphenyl)methyl]but-2-enoate (350 mg, 1.2 mmol, 37.8% yield) was obtained as a colorless oil.

¹H NMR (400 MHz, DMSO-d₆) δ=7.12 (d, J=8.4 Hz, 2H), 6.89 (d, J=8.4 Hz, 2H), 5.75 (s, 1H), 4.21 (q, J=7.2 Hz, 2H), 3.82 (s, 3H), 3.53 (s, 2H), 1.29 (t, J=7.2 Hz, 3H).

### Step C. Procedure for Preparation of ethyl 4,4,4-trifluoro-3-[(4-methoxyphenyl)methyl]butanoate

Ethyl (Z)-4,4,4-trifluoro-3-[(4-methoxyphenyl)methyl]but-2-enoate (350 mg, 1.21 mmol, 1 equiv.), Pd/C (100 mg, 10% purity) in EtOH (5 mL) was degassed and purged with H₂ three times, and then the mixture was stirred at 25° C. for 2 hours under H₂ (15 Psi). The reaction mixture was filtered and concentrated under reduced pressure to give a residue. The residue was used in the next step without other purification. Ethyl 4,4,4-trifluoro-3-[(4-methoxyphenyl)methyl]butanoate (550 mg, crude) was obtained as a colorless oil.

¹H NMR (400 MHz, DMSO-d₆) δ=7.13 (d, J=8.4 Hz, 2H), 6.85 (d, J=8.4 Hz, 2H), 4.03 (q, J=7.2 Hz, 2H), 3.80 (s, 3H), 3.10-2.97 (m, 2H), 2.66-2.49 (m, 2H), 2.42-2.30 (m, 1H), 1.20 (t, J=7.2 Hz, 3H).

### Step D. Procedure for Preparation of ethyl 4,4,4-trifluoro-3-[(4-hydroxyphenyl)methyl]butanoate

To a solution of ethyl 4,4,4-trifluoro-3-[(4-methoxyphenyl)methyl]butanoate (550 mg, 1.89 mmol, 1 equiv.) in DCM (10 mL) was added BBr₃ (2.37 g, 9.47 mmol, 912.83 μL, 5 equiv.) under −78° C. The mixture was stirred at 20° C. for 2 hours. After cooling to 0° C., the reaction was slowly quenched with EtOH (40 mL). Then filtered and concentrated under reduced pressure to obtain a residue. The residue was purified by flash silica gel chromatography (eluent of 0˜10% ethyl acetate/petroleum ether). Ethyl 4,4,4-trifluoro-3-[(4-hydroxyphenyl)methyl]butanoate (470 mg, 1.7 mmol, 89.7% yield) was obtained as a colorless oil.

¹H NMR (400 MHz, DMSO-d₆) δ=7.08 (d, J=8.4 Hz, 2H), 6.80-6.75 (m, 2H), 4.09-3.98 (m, 2H), 3.08-2.96 (m, 2H), 2.64-2.49 (m, 2H), 2.36 (dd, J=6.4, 16.4 Hz, 1H), 1.21 (t, J=7.2 Hz, 3H).

### Step E. Procedure for Preparation of ethyl 3-[[4-(3-bromo-2-methyl-phenoxy)phenyl]methyl]-4,4,4-trifluoro-butanoate

A mixture of ethyl 4,4,4-trifluoro-3-[(4-hydroxyphenyl)methyl]butanoate (470 mg, 1.70 mmol, 1 equiv.), 1,3-dibromo-2-methyl-benzene (1.28 g, 5.10 mmol, 3 equiv.), Cs₂CO₃ (665.20 mg, 2.04 mmol, 1.2 equiv.), CuI (162.01 mg, 850.68 μmol, 0.5 equiv.), and 2,2,6,6-tetramethylheptane-3,5-dione (78.38 mg, 425.34 μmol, 87.57 μL, 0.25 equiv.) in NMIP (5 mL) was degassed and purged with N₂ three times. The mixture was then stirred at 120° C. for 12 hours under N₂ atmosphere. The reaction mixture was quenched by the addition of saturated NH₄Cl (10 mL) at 0° C., diluted with EtOAc (10 mL), and extracted with EtOAc (10 mL×3). The combined organic layers were filtered and concentrated under reduced pressure to give a residue. The residue was purified by flash silica gel chromatography (eluent of 0˜20% ethyl acetate/petroleum ether). Ethyl 3-[[4-(3-bromo-2-methyl-phenoxy)phenyl]methyl]-4,4,4-trifluoro-butanoate (530 mg, 1.1 mmol, 69.9% yield) was obtained as a yellow oil.

¹H NMR (400 MHz, DMSO-d₆) δ=7.37 (dd, J=0.8, 8.0 Hz, 1H), 7.17-7.14 (m, 2H), 7.03 (t, J=8.0 Hz, 1H), 6.86-6.83 (m, 3H), 4.09-4.03 (m, 2H), 3.08-3.02 (m, 2H), 2.65-2.54 (m, 2H), 2.40-2.36 (m, 1H), 2.33 (s, 3H), 1.22 (t, J=7.2 Hz, 3H)

### Step F. Procedure for Preparation of ethyl (3S)-3-[[4-(3-bromo-2-methyl-phenoxy)phenyl]methyl]-4,4,4-trifluoro-butanoate

The stereoisomers of ethyl 3-[[4-(3-bromo-2-methyl-phenoxy)phenyl]methyl]-4,4,4-trifluoro-butanoate were separated by SFC to provide ethyl (3S)-3-[[4-(3-bromo-2-methyl-phenoxy)phenyl]methyl]-4,4,4-trifluoro-butanoate (158 mg, 354.84 μmol, 29.81% yield) as a yellow oil, and ethyl (R)-3-(4-(3-bromo-2-methylphenoxy)benzyl)-4,4,4-trifluorobutanoate (66 mg, 96.3 μmol, 14.3% yield) as a yellow oil.

### Step G. Procedure for Preparation of tert-butyl 6-[8-(1,3-benzothiazol-2-ylcarbamoyl)-3,4-dihydro-1H-isoquinolin-2-yl]-3-[3-[4-[(2S)-4-ethoxy-4-oxo-2-(trifluoromethyl)butyl]phenoxy]-2-methyl-phenyl]pyridine-2-carboxylate

A mixture of ethyl (3S)-3-[[4-(3-bromo-2-methyl-phenoxy)phenyl]methyl]-4,4,4-trifluoro-butanoate (138 mg, 309.92 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (227.81 mg, 371.91 μmol, 1.2 equiv.), [2-(2-aminophenyl)phenyl]palladium(1+);bis(1-adamantyl)-butyl-phosphane;methanesulfonate (45.14 mg, 61.98 μmol, 0.2 equiv.), and K₂CO₃ (1.5 M, 309.92 μL, 1.5 equiv.) in dioxane (1.5 mL) was degassed and purged with N₂ three times. The mixture was then stirred at 100° C. for 1 hour under N₂ atmosphere. The combined organic layers were filtered and concentrated under reduced pressure to give a residue. The residue was purified by flash silica gel chromatography (eluent of 0˜8% ethyl acetate/petroleum ether). Tert-butyl 6-[8-(1,3-benzothiazol-2-ylcarbamoyl)-3,4-dihydro-1H-isoquinolin-2-yl]-3-[3-[4-[(2S)-4-ethoxy-4-oxo-2-(trifluoromethyl)butyl]phenoxy]-2-methyl-phenyl]pyridine-2-carboxylate (220 mg, 214.5 μmol, 69.2% yield, 83% purity) was obtained as a yellow oil.

MS (ESI) m/z: 851.4 [M+H]⁺.

### Step H. Procedure for Preparation of (3S)-3-[[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]phenyl]methyl]-4,4,4-trifluoro-butanoic acid

A mixture of tert-butyl 6-[8-(1,3-benzothiazol-2-ylcarbamoyl)-3,4-dihydro-1H-isoquinolin-2-yl]-3-[3-[4-[(2S)-4-ethoxy-4-oxo-2-(trifluoromethyl)butyl]phenoxy]-2-methyl-phenyl]pyridine-2-carboxylate (220 mg, 258.54 μmol, 1 equiv.), and lithium hydroxide hydrate (54.25 mg, 1.29 mmol, 5 equiv.) in THE (3 mL) and water (1 mL) was stirred at 25° C. for 16 hours. The reaction mixture was diluted with water (5 mL) and concentrated into a turbid liquid. Then the pH of the turbid liquid was adjusted to 3 with 1 N HCl (5 mL), and the residue was extracted with DCM/MeOH (20:1). The combined organic layers were filtered and concentrated. The residue was used in the next step without further purification. (3S)-3-[[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]phenyl]methyl]-4,4,4-trifluoro-butanoic acid (150 mg, 182.2 μmol, 70.5% yield) was obtained as a yellow oil.

MS (ESI) m/z: 823.5 [M+H]⁺.

### Step I. Procedure for Preparation of tert-butyl 6-[8-(1,3-benzothiazol-2-ylcarbamoyl)-3,4-dihydro-1H-isoquinolin-2-yl]-3-[3-[4-[(2S)-4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-4-oxo-2-(trifluoromethyl)butyl]phenoxy]-2-methyl-phenyl]pyridine-2-carboxylate

A mixture of (3S)-3-[[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]phenyl]methyl]-4,4,4-trifluoro-butanoic acid (100 mg, 121.52 μmol, 1 equiv.), (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (64.83 mg, 145.83 μmol, 1.2 equiv.), HATU (69.31 mg, 182.28 μmol, 1.5 equiv.), and DIEA (47.12 mg, 364.57 μmol, 63.50 μL, 3 equiv.) in DMF (2 mL) was stirred at 25° C. for 0.5 hour. Then 3-(6-amino-1-methyl-indazol-3-yl)piperidine-2,6-dione (7.87 mg, 30.46 μmol, 1.2 equiv.) was added to the mixture, and the mixture was stirred at 25° C. for 1.5 hours. The reaction mixture was washed with saturated NaCl (5 mL×2) and concentrated into a turbid liquid. Then the residue was extracted with ethyl acetate (10 mL×3). The combined organic layers were filtered and concentrated. The residue was used in the next step without further purification. Tert-butyl 6-[8-(1,3-benzothiazol-2-ylcarbamoyl)-3,4-dihydro-1H-isoquinolin-2-yl]-3-[3-[4-[(2S)-4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-4-oxo-2-(trifluoromethyl)butyl]phenoxy]-2-methyl-phenyl]pyridine-2-carboxylate (110 mg, 88.0 μmol, 72.4% yield) was obtained as a yellow oil.

MS (ESI) m/z: 1249.5 [M+H]⁺.

### Step J. Procedure for Preparation of 6-[8-(1,3-benzothiazol-2-ylcarbamoyl)-3,4-dihydro-1H-isoquinolin-2-yl]-3-[3-[4-[(2S)-4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-4-oxo-2-(trifluoromethyl)butyl]phenoxy]-2-methyl-phenyl]pyridine-2-carboxylic acid [6-(8-(benzo[d]thiazol-2-ylcarbamoyl)-3,4-dihydroisoquinolin-2 (1H)-yl)-3-(3-(4-((S*)-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxo-2-(trifluoromethyl)butyl)phenoxy)-2-methylphenyl)picolinic acid]

A solution of tert-butyl 6-[8-(1,3-benzothiazol-2-ylcarbamoyl)-3,4-dihydro-1H-isoquinolin-2-yl]-3-[3-[4-[(2S)-4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-4-oxo-2-(trifluoromethyl)butyl]phenoxy]-2-methyl-phenyl]pyridine-2-carboxylate (70 mg, 56.02 μmol, 1 equiv.) in TFA (0.8 mL) and DCM (0.8 mL) was stirred at 25° C. for 2 hours. The reaction mixture was concentrated under reduced pressure to give a residue. The residue was purified by prep-HPLC. 6-[8-(1,3-Benzothiazol-2-ylcarbamoyl)-3,4-dihydro-1H-isoquinolin-2-yl]-3-[3-[4-[(2S)-4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-4-oxo-2-(trifluoromethyl)butyl]phenoxy]-2-methyl-phenyl]pyridine-2-carboxylic acid [6-(8-(benzo[d]thiazol-2-ylcarbamoyl)-3,4-dihydroisoquinolin-2 (1H)-yl)-3-(3-(4-((S*)-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxo-2-(trifluoromethyl)butyl)phenoxy)-2-methylphenyl)picolinic acid](28.2 mg, 22.5 mol, 40.2% yield, 95.3% purity) was obtained as a yellow solid.

MS (ESI) m/z: 1193.4 [M+H]⁺.

¹H NMR (400 MHz, DMSO-d₆) δ=13.08-12.56 (m, 2H), 8.98 (s, 1H), 8.37 (d, J=8.4 Hz, 1H), 8.17-8.11 (m, 1H), 8.05-8.01 (m, 1H), 7.79 (d, J=8.4 Hz, 1H), 7.66-7.60 (m, 1H), 7.56-7.51 (m, 1H), 7.50-7.25 (m, 9H), 7.22-7.18 (m, 2H), 6.99 (d, J=8.0 Hz, 1H), 6.94-6.76 (m, 4H), 4.99 (s, 2H), 4.90 (t, J=7.2 Hz, 1H), 4.53-4.47 (m, 1H), 4.39 (t, J=7.6 Hz, 1H), 4.30-4.22 (m, 1H), 3.98-3.87 (m, 2H), 3.62-3.54 (m, 2H), 3.09-2.99 (m, 3H), 2.91-2.81 (m, 1H), 2.69-2.59 (m, 2H), 2.44 (s, 3H), 2.23-2.15 (m, 1H), 2.10-1.94 (m, 2H), 1.90 (s, 3H), 1.80-1.71 (m, 1H), 1.35 (d, J=7.2 Hz, 3H), 0.93 (s, 9H)

19F NMR (400 MHz, DMSO-d₆) δ=−69.26

### Example 34. Preparation of Compound 131b

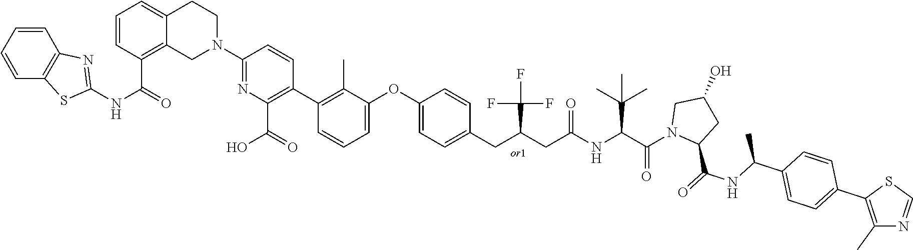 [SMILES: Cc1ncsc1-c1ccc([C@H](C)NC(=O)[C@@H]2C[C@@H](O)CN2C(=O)[C@@H](NC(=O)C[C@@H](Cc2ccc(Oc3cccc(-c4ccc(N5CCc6cccc(C(=O)Nc7nc8ccccc8s7)c6C5)nc4C(=O)O)c3C)cc2)C(F)(F)F)C(C)(C)C)cc1]

### 6-(8-(benzo[d]thiazol-2-ylcarbamoyl)-3,4-dihydroisoquinolin-2 (1H)-yl)-3-(3-(4-((R)-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxo-2-(trifluoromethyl)butyl)phenoxy)-2-methylphenyl)picolinic acid [6-(8-(benzo[d]thiazol-2-ylcarbamoyl)-3,4-dihydroisoquinolin-2 (1H)-yl)-3-(3-(4-((R*)-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxo-2-(trifluoromethyl)butyl)phenoxy)-2-methylphenyl)picolinic acid]

### Step A. Procedure for Preparation of tert-butyl (R)-6-(8-(benzo[d]thiazol-2-ylcarbamoyl)-3,4-dihydroisoquinolin-2 (1H)-yl)-3-(3-(4-(4-ethoxy-4-oxo-2-(trifluoromethyl)butyl)phenoxy)-2-methylphenyl)picolinate

A mixture of ethyl (R)-3-(4-(3-bromo-2-methylphenoxy)benzyl)-4,4,4-trifluorobutanoate (66 mg, 96.35 μmol, 65% purity, 1 equiv.), tert-butyl 6-(8-(benzo[d]thiazol-2-ylcarbamoyl)-3,4-dihydroisoquinolin-2 (1H)-yl)-3-(4,4,5,5-tetramethyl-1,3,2-dioxaborolan-2-yl)picolinate (70.82 mg, 115.62 μmol, 1.2 equiv.), [2-(2-aminophenyl)phenyl]palladium(1+);bis(1-adamantyl)-butyl-phosphane;methanesulfonate (14.03 mg, 19.27 μmol, 0.2 equiv.), and K₂CO₃ (1.5 M, 96.35 μL, 1.5 equiv.) in dioxane (1 mL) was degassed and purged with N₂ three times. The mixture was then stirred at 100° C. for 1 hour under N₂ atmosphere. The reaction was diluted with water (20 mL) and extracted with DCM (20 mL×2). The combined organic layers were washed with brine (30 mL), dried over sodium sulfate, filtered, and concentrated under reduced pressure to give a residue. The residue was purified by flash silica gel chromatography (eluent of 0˜8% ethyl acetate/petroleum ether). Tert-butyl (R)-6-(8-(benzo[d]thiazol-2-ylcarbamoyl)-3,4-dihydroisoquinolin-2 (1H)-yl)-3-(3-(4-(4-ethoxy-4-oxo-2-(trifluoromethyl)butyl)phenoxy)-2-methylphenyl)picolinate (80 mg, 78.0 μmol, 80.9% yield, 83% purity) was obtained as a yellow oil.

MS (ESI) m/z: 851.5 [M+H]⁺

### Step B. Procedure for Preparation of (R)-3-(4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)benzyl)-4,4,4-trifluorobutanoic acid

A mixture of tert-butyl (R)-6-(8-(benzo[d]thiazol-2-ylcarbamoyl)-3,4-dihydroisoquinolin-2 (1H)-yl)-3-(3-(4-(4-ethoxy-4-oxo-2-(trifluoromethyl)butyl)phenoxy)-2-methylphenyl)picolinate (80 mg, 94.01 μmol, 1 equiv.), lithium hydroxide hydrate (19.73 mg, 470.07 μmol, 5 equiv.), THE (3 mL), and water (1 mL) was stirred at 25° C. for 16 hours. The reaction mixture was diluted with water (5 mL) and concentrated to provide a turbid liquid. Then the pH of the turbid liquid was adjusted to 3 with 1 N HCl (5 mL), and the residue was extracted with DCM/MeOH (20:1). The combined organic layers were filtered and concentrated. The residue was used in the next step without further purification. (R)-3-(4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)benzyl)-4,4,4-trifluorobutanoic acid (80 mg, crude) was obtained as a yellow oil.

MS (ESI) m/z: 823.7 [M+H]⁺

### Step C. Procedure for Preparation of tert-butyl 6-(8-(benzo[d]thiazol-2-ylcarbamoyl)-3,4-dihydroisoquinolin-2 (1H)-yl)-3-(3-(4-((R)-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxo-2-(trifluoromethyl)butyl)phenoxy)-2-methylphenyl)picolinate

A mixture of (R)-3-(4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)benzyl)-4,4,4-trifluorobutanoic acid (80 mg, 97.22 μmol, 1 equiv.), (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (51.87 mg, 116.66 μmol, 1.2 equiv.), HATU (55.45 mg, 145.83 μmol, 1.5 equiv.), and DIEA (37.69 mg, 291.66 μmol, 50.80 μL, 3 equiv.) in DMF (2 mL) was degassed and purged with N₂ three times. The mixture was then stirred at 25° C. for 2 hours under N₂ atmosphere. The reaction was diluted with water (20 mL) and extracted with DCM (20 mL×2). The combined organic layers were washed with brine (30 mL), dried over sodium sulfate, filtered, and concentrated under reduced pressure to give a residue. The residue was used in the next step without further purification. Tert-butyl 6-(8-(benzo[d]thiazol-2-ylcarbamoyl)-3,4-dihydroisoquinolin-2 (1H)-yl)-3-(3-(4-((R)-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxo-2-(trifluoromethyl)butyl)phenoxy)-2-methylphenyl)picolinate (80 mg, 64.0 μmol, 65.8% yield) was obtained as a yellow oil.

MS (ESI) m/z: 1249.4[M+H]⁺

### Step D. Procedure for Preparation of 6-(8-(benzo[d]thiazol-2-ylcarbamoyl)-3,4-dihydroisoquinolin-2 (1H)-yl)-3-(3-(4-((R)-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxo-2-(trifluoromethyl)butyl)phenoxy)-2-methylphenyl)picolinic acid [6-(8-(benzo[d]thiazol-2-ylcarbamoyl)-3,4-dihydroisoquinolin-2 (1H)-yl)-3-(3-(4-((R*)-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxo-2-(trifluoromethyl)butyl)phenoxy)-2-methylphenyl)picolinic acid]

To a solution of tert-butyl 6-(8-(benzo[d]thiazol-2-ylcarbamoyl)-3,4-dihydroisoquinolin-2 (1H)-yl)-3-(3-(4-((R)-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxo-2-(trifluoromethyl)butyl)phenoxy)-2-methylphenyl)picolinate (80 mg, 64.03 μmol, 1 equiv.) in TFA (0.8 mL), DCM (0.8 mL) was added. The mixture was stirred at 25° C. for 2 hours. The reaction was diluted with water (20 mL) and extracted with DCM 20 mL (20 mL×2). The combined organic layers were washed with brine (30 mL), dried over sodium sulfate, filtered, and concentrated under reduced pressure to give as a residue. The residue was purified by prep-HPLC. 6-(8-(Benzo[d]thiazol-2-ylcarbamoyl)-3,4-dihydroisoquinolin-2 (1H)-yl)-3-(3-(4-((R)-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxo-2-(trifluoromethyl)butyl)phenoxy)-2-methylphenyl)picolinic acid [6-(8-(benzo[d]thiazol-2-ylcarbamoyl)-3,4-dihydroisoquinolin-2 (1H)-yl)-3-(3-(4-((R*)-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxo-2-(trifluoromethyl)butyl)phenoxy)-2-methylphenyl)picolinic acid](17.56 mg, 13.8 mol, 21.6% yield, 94.3% purity) was obtained as a yellow solid.

MS (ESI) m/z: 1193.4 [M+H]⁺.

¹H NMR (400 MHz, DMSO-d₆) δ=12.96-12.40 (m, 1H), 9.04-8.92 (m, 1H), 8.45-8.33 (m, 1H), 8.20-8.09 (m, 1H), 8.03 (d, J=8.0 Hz, 1H), 7.79 (d, J=8.0 Hz, 1H), 7.62 (d, J=7.2 Hz, 1H), 7.55-7.33 (m, 9H), 7.24-7.12 (m, 3H), 6.99 (d, J=8.8 Hz, 1H), 6.90 (d, J=7.6 Hz, 1H), 6.88-6.75 (m, 3H), 4.99 (s, 2H), 4.94-4.86 (m, 1H), 4.50 (d, J=9.2 Hz, 1H), 4.45-4.27 (m, 2H), 3.96-3.88 (m, 2H), 3.63-3.57 (m, 2H), 3.03 (t, J=5.2 Hz, 3H), 2.94-2.73 (m, 2H), 2.71-2.55 (m, 2H), 2.46-2.43 (m, 3H), 2.32-2.10 (m, 1H), 2.03-1.96 (m, 1H), 1.92-1.85 (m, 3H), 1.81-1.73 (m, 1H), 1.43-1.31 (m, 3H), 0.92 (d, J=11.2 Hz, 9H).

### Example 35. Preparation of Compound 132a

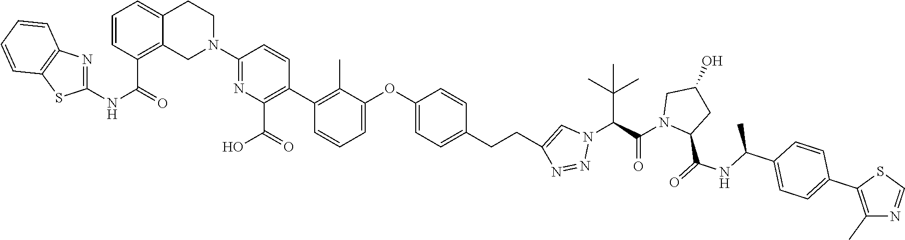 [SMILES: Cc1ncsc1-c1ccc([C@H](C)NC(=O)[C@@H]2C[C@@H](O)CN2C(=O)[C@@H](n2cc(CCc3ccc(Oc4cccc(-c5ccc(N6CCc7cccc(C(=O)Nc8nc9ccccc9s8)c7C6)nc5C(=O)O)c4C)cc3)nn2)C(C)(C)C)cc1]

### 6-(8-(benzo[d]thiazol-2-ylcarbamoyl)-3,4-dihydroisoquinolin-2 (1H)-yl)-3-(3-(4-(2-(1-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-1H-1,2,3-triazol-4-yl)ethyl)phenoxy)-2-methylphenyl)picolinic acid

### Step A. Procedure for Preparation of ethyl (E)-3-(4-hydroxyphenyl)acrylate

A mixture of NaH (3.44 g, 85.98 mmol, 60% purity, 2.1 equiv.) in THE (100 mL) was purged with N₂ three times. To the mixture was slowly added ethyl 2-(diethoxyphosphoryl)acetate (18.36 g, 81.89 mmol, 16.25 mL, 2.0 equiv.) at 0° C., and then the mixture was stirred for 1.5 hours under N₂ atmosphere. To the reaction mixture was added 4-hydroxybenzaldehyde (5 g, 40.94 mmol, 1.0 equiv.) at 0° C. and warm to 25° C., and the resulting mixture was stirred for 1.5 hours under N₂ atmosphere. The reaction mixture was quenched by addition saturated NH₄Cl (150 mL) under 0° C. and extracted with DCM (100 mL×3). The combined organic layers were washed with brine (100 mL×2), dried over sodium sulfate, filtered, and concentrated under reduced pressure to give a residue. The residue was purified by flash silica gel chromatography (eluent of 0˜8% ethyl acetate/petroleum ether) to give ethyl (E)-3-(4-hydroxyphenyl)acrylate (2.4 g, 12.4 mmol, 30.5% yield) as a white solid.

¹H NMR (400 MHz, DMSO-d₆) δ=10.23-9.80 (m, 1H), 7.55 (d, J=8.4 Hz, 3H), 6.79 (d, J=7.6 Hz, 2H), 6.39 (d, J=16.0 Hz, 1H), 4.16 (q, J=6.8 Hz, 2H), 1.35-1.14 (m, 3H)

### Step B. Procedure for Preparation of ethyl 3-(4-hydroxyphenyl)propanoate

To a solution of ethyl (E)-3-(4-hydroxyphenyl)acrylate (2.40 g, 12.49 mmol, 1.0 equiv.) in THF (30 mL) was added PtO₂ (567.07 mg, 2.50 mmol, 0.2 equiv.) under H₂ atmosphere (15 psi). The mixture was stirred at 25° C. for 16 hours. The reaction mixture was filtered and concentrated under reduced pressure to give a residue. The residue was purified by flash silica gel chromatography (eluent of 0˜8% ethyl acetate/petroleum ether) to give ethyl 3-(4-hydroxyphenyl)propanoate (2.1 g, 10.3 mmol, 82.8% yield, 95.6% purity) as a colorless oil.

¹H NMR (400 MHz, CDCl₃) δ=7.17-7.01 (m, 2H), 6.85-6.68 (m, 2H), 4.17-4.12 (m, 2H), 2.90 (t, J=7.8 Hz, 2H), 2.63-2.56 (m, 2H), 1.29-1.25 (m, 3H)

### Step C. Procedure for Preparation of ethyl 3-(4-(3-bromo-2-methylphenoxy)phenyl)propanoate

A mixture of 1,3-dibromo-2-methylbenzene (5.40 g, 21.62 mmol, 2.0 equiv.), ethyl 3-(4-hydroxyphenyl)propanoate (2.1 g, 10.81 mmol, 1.0 equiv.), CuI (1.03 g, 5.41 mmol, 0.5 equiv.), Cs₂CO₃ (4.23 g, 12.97 mmol, 1.2 equiv.), and 2,2,6,6-tetramethylheptane-3,5-dione (498.11 mg, 2.70 mmol, 556.55 μL, 0.25 equiv.) in NMP (20 mL) was degassed and purged with N₂ three times. Then the mixture was stirred at 120° C. for 12 hours under N₂ atmosphere. The reaction mixture was concentrated under reduced pressure to remove solvent. The residue was purified by flash silica gel chromatography (eluent of 0˜3% ethyl acetate/petroleum ether) to give ethyl 3-(4-(3-bromo-2-methylphenoxy)phenyl)propanoate (3.33 g, 4.5 mmol, 42.0% yield, 49.5% purity) as a yellow oil.

¹H NMR (400 MHz, DMSO-d₆) δ=7.43 (dd, J=0.8, 8.0 Hz, 1H), 7.24-7.20 (m, 2H), 7.16 (t, J=8.0 Hz, 1H), 6.88-6.81 (m, 3H), 4.04 (q, J=7.2 Hz, 2H), 2.85-2.80 (m, 2H), 2.62-2.58 (m, 2H), 2.26 (s, 3H), 1.15 (t, J=7.2 Hz, 3H)

### Step D. Procedure for Preparation of 3-(4-(3-bromo-2-methylphenoxy)phenyl)propan-1-ol

A mixture of ethyl 3-(4-(3-bromo-2-methylphenoxy)phenyl)propanoate (3.33 g, 9.17 mmol, 1.0 equiv.), LiAlH₄ (347.90 mg, 9.17 mmol, 1.0 equiv.) in THE (30 mL) was degassed and purged with N₂ for 3 times, and then the mixture was stirred at 0° C. for 1 hour under N₂ atmosphere. The reaction mixture was quenched by addition water 0.4 m1 and 15% NaOH 0.4 m1 and water 1.2 m1. The combined mixture was mixed with sodium sulfate, then filtered and concentrated under reduced pressure to give 3-(4-(3-bromo-2-methylphenoxy)phenyl)propan-1-ol (2 g, 6.2 mmol, 67.9% yield) as a yellow oil.

¹H NMR (400 MHz, CDCl₃) δ=7.26 (d, J=8.0 Hz, 1H), 7.19 (s, 1H), 7.09-7.04 (m, 2H), 7.00-6.90 (m, 1H), 6.77-6.74 (m, 2H), 3.61 (t, J=6.4 Hz, 2H), 2.64-2.59 (m, 2H), 2.29-2.17 (m, 3H), 1.85-1.77 (m, 2H)

### Step E. Procedure for Preparation of 3-(4-(3-bromo-2-methylphenoxy)phenyl)propanal

To a solution of (COCl)₂ (790.30 mg, 6.23 mmol, 545.04 μL, 2.0 equiv.) in DCM (5 mL) was added dropwise to a solution of DMSO (972.99 mg, 12.45 mmol, 972.99 μL, 4.0 equiv.) in DCM (5 mL) at −70° C. under N₂ atmosphere. The mixture was stirred at −70° C. for 1 hour. After which time 3-(4-(3-bromo-2-methylphenoxy)phenyl)propan-1-ol (1 g, 3.11 mmol, 1.0 equiv.) in DCM (15 mL) was added dropwise at −70° C. The solution was stirred for 1 hour at −70° C. Then TEA (1.89 g, 18.68 mmol, 2.60 mL, 6.0 equiv.) was added into solution. The solution was stirred at −70° C. for 1 hour under N₂ atmosphere. The reaction was diluted with water (20 mL) and extracted with DCM 50 mL (50 mL×2). The combined organic layers were washed with brine 50 mL (50 mL×1), dried over sodium sulfate, filtered and concentrated under reduced pressure to give 3-(4-(3-bromo-2-methylphenoxy)phenyl)propanal (900 mg, 2.82 mmol, 90.57% yield) as a yellow oil.

¹H NMR (400 MHz, CDCl₃ δ=9.79-9.71 (m, 1H), 7.27 (d, J=8.0 Hz, 1H), 7.19 (s, 1H), 7.08-7.03 (m, 2H), 6.99-6.91 (m, 1H), 6.77-6.74 (m, 2H), 2.89-2.83 (m, 2H), 2.73-2.67 (m, 2H), 2.28-2.22 (m, 3H)

### Step F. Procedure for Preparation of 1-bromo-3-(4-but-3-ynylphenoxy)-2-methyl-benzene

To a solution of 3-(4-(3-bromo-2-methylphenoxy)phenyl)propanal (900 mg, 2.82 mmol, 1 equiv.) in MeOH (10 mL) was added K₂CO₃ (1.17 g, 8.46 mmol, 3.0 equiv.) and dimethyl (1-diazo-2-oxopropyl)phosphonate (541.67 mg, 2.82 mmol, 1.0 equiv.). The mixture was stirred at 25° C. for 1 hour. The reaction mixture was concentrated under reduced pressure to give a residue. The residue was purified by flash silica gel chromatography (eluent of 0˜2% ethyl acetate/petroleum ether) to give 1-bromo-3-(4-but-3-ynylphenoxy)-2-methyl-benzene (360 mg, 935.1 μmol, 33.1% yield, 81.8% purity) as a yellow oil.

¹H NMR (400 MHz, DMSO-d₆) δ=7.43 (d, J=8.0 Hz, 1H), 7.26 (d, J=8.4 Hz, 2H), 7.16 (t, J=8.0 Hz, 1H), 6.90-6.85 (m, 3H), 2.79 (t, J=2.4 Hz, 1H), 2.74 (t, J=7.2 Hz, 2H), 2.46-2.42 (m, 2H), 2.27 (s, 3H)

### Step G. Procedure for Preparation of (2S,4R)-1-((S)-2-(4-(4-(3-bromo-2-methylphenoxy)phenethyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

A mixture of 1-bromo-3-(4-but-3-ynylphenoxy)-2-methyl-benzene (360 mg, 1.14 mmol, 1.0 equiv.), (2S,4R)-1-((S)-2-azido-3,3-dimethylbutanoyl)-4-hydroxy-N—((S)-1-(4-(4-methylthiazol-5-yl)phenyl)ethyl)pyrrolidine-2-carboxamide (537.47 mg, 1.14 mmol, 1.0 equiv.), sodium-L-aspartate (354.26 mg, 2.28 mmol, 2.0 equiv.), CuSO₄ (364.59 mg, 2.28 mmol, 350.56 μL, 2.0 equiv.) and MeOH (4 mL) in water (4 mL) was degassed and purged with N₂ three times, and then the mixture was stirred at 25° C. for 12 hours under N₂ atmosphere. The reaction mixture was concentrated under reduced pressure to remove solvent. The residue was purified by prep-TLC (SiO₂, petroleum ether/ethyl acetate=0/1) to give (2S,4R)-1-((S)-2-(4-(4-(3-bromo-2-methylphenoxy)phenethyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide (280 mg, 356.3 μmol, 31.2% yield) as a white solid.

MS (ESI) m/z: 785.2 [M+H]⁺.

### Step H. Procedure for Preparation of tert-butyl 6-(8-(benzo[d]thiazol-2-ylcarbamoyl)-3,4-dihydroisoquinolin-2 (1H)-yl)-3-(3-(4-(2-(1-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-1H-1,2,3-triazol-4-yl)ethyl)phenoxy)-2-methylphenyl)picolinate

A mixture of (2S,4R)-1-((S)-2-(4-(4-(3-bromo-2-methylphenoxy)phenethyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide (80 mg, 101.81 μmol, 1.2 equiv.), tert-butyl 6-(8-(benzo[d]thiazol-2-ylcarbamoyl)-3,4-dihydroisoquinolin-2 (1H)-yl)-3-(4,4,5,5-tetramethyl-1,3,2-dioxaborolan-2-yl)picolinate (51.97 mg, 84.84 μmol, 1.0 equiv.), Ad₂nBup-Pd-G₃ (6.18 mg, 8.48 μmol, 0.1 equiv.), KF (14.79 mg, 254.52 μmol, 5.96 μL, 3.0 equiv.), and water (0.5 mL) in dioxane (1 mL) was degassed and purged with N₂ three times, and then the mixture was stirred at 100° C. for 1 hour under N₂ atmosphere. The reaction mixture was concentrated under reduced pressure to remove solvent. The residue was purified by prep-TLC (SiO₂, DCM:MeOH=10:1) to give tert-butyl 6-(8-(benzo[d]thiazol-2-ylcarbamoyl)-3,4-dihydroisoquinolin-2 (1H)-yl)-3-(3-(4-(2-(1-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-1H-1,2,3-triazol-4-yl)ethyl)phenoxy)-2-methylphenyl)picolinate (80 mg, 67.1 μmol, 79.1% yield) as a yellow solid.

MS (ESI) m/z: 1191.4 [M+H]⁺.

### Step I. Procedure for Preparation of 6-(8-(benzo[d]thiazol-2-ylcarbamoyl)-3,4-dihydroisoquinolin-2 (1H)-yl)-3-(3-(4-(2-(1-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-1H-1,2,3-triazol-4-yl)ethyl)phenoxy)-2-methylphenyl)picolinic acid

To a solution of tert-butyl 6-(8-(benzo[d]thiazol-2-ylcarbamoyl)-3,4-dihydroisoquinolin-2 (1H)-yl)-3-(3-(4-(2-(1-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-1H-1,2,3-triazol-4-yl)ethyl)phenoxy)-2-methylphenyl)picolinate (80 mg, 67.14 μmol, 1.0 equiv.) in DCM (1 mL) was added TFA (7.66 mg, 67.14 μmol, 4.97 μL, 1.0 equiv.). The mixture was stirred at 25° C. for 12 hours. The reaction mixture was concentrated under reduced pressure to remove solvent. The residue was purified by prep-HPLC to give 6-(8-(benzo[d]thiazol-2-ylcarbamoyl)-3,4-dihydroisoquinolin-2 (1H)-yl)-3-(3-(4-(2-(1-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-1H-1,2,3-triazol-4-yl)ethyl)phenoxy)-2-methylphenyl)picolinic acid (21.7 mg, 18.5 mol, 27.5% yield, 96.5% purity) as a yellow solid.

MS (ESI) m/z: 1135.4 [M+H]⁺.

¹H NMR (400 MHz, DMSO-d₆) δ=δ=12.87 (s, 1H), 12.76-12.56 (m, 1H), 8.99 (s, 1H), 8.49 (d, J=7.6 Hz, 1H), 8.04 (d, J=7.6 Hz, 1H), 7.92-7.84 (m, 1H), 7.80 (d, J=7.6 Hz, 1H), 7.63 (d, J=6.4 Hz, 1H), 7.54 (d, J=8.8 Hz, 1H), 7.50-7.42 (m, 4H), 7.41-7.33 (m, 4H), 7.21-7.14 (m, 3H), 7.02 (d, J=8.8 Hz, 1H), 6.89 (d, J=7.6 Hz, 1H), 6.86-6.77 (m, 3H), 5.41 (s, 1H), 5.00 (s, 2H), 4.96-4.87 (m, 1H), 4.42 (t, J=8.4 Hz, 1H), 4.29 (s, 1H), 3.94 (t, J=5.6 Hz, 2H), 3.72-3.53 (m, 2H), 3.04 (t, J=5.6 Hz, 2H), 2.99-2.82 (m, 4H), 2.46 (s, 3H), 2.07 (dd, J=8.68, 11.7 Hz, 1H), 1.91 (s, 3H), 1.82-1.73 (m, 1H), 1.38 (d, J=7.2 Hz, 3H), 1.28-1.16 (m, 1H), 0.93 (s, 9H)

### Example 36. Preparation of Compound 133a

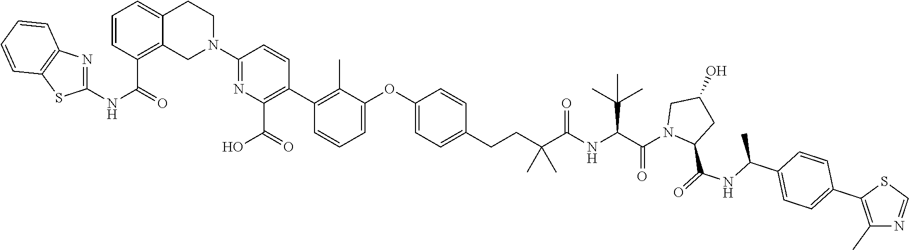 [SMILES: Cc1ncsc1-c1ccc([C@H](C)NC(=O)[C@@H]2C[C@@H](O)CN2C(=O)[C@@H](NC(=O)C(C)(C)CCc2ccc(Oc3cccc(-c4ccc(N5CCc6cccc(C(=O)Nc7nc8ccccc8s7)c6C5)nc4C(=O)O)c3C)cc2)C(C)(C)C)cc1]

### 6-[8-(1,3-benzothiazol-2-ylcarbamoyl)-3,4-dihydro-1H-isoquinolin-2-yl]-3-[3-[4-[4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3,3-dimethyl-4-oxo-butyl]phenoxy]-2-methyl-phenyl]pyridine-2-carboxylic acid

### Step A. Procedure for Preparation of methyl 4-bromo-2,2-dimethyl-butanoate

To a solution of 3,3-dimethyltetrahydrofuran-2-one (4.5 g, 39.42 mmol, 1.0 equiv.) in DCM (50 mL) was added BBr₃ (19.75 g, 78.85 mmol, 7.60 mL, 2.0 equiv.). The mixture was stirred at −78° C. for 12 hours. After cooling to 0° C., the reaction was slowly quenched with MeOH (40 mL). The mixture was stirred at −78° C. for 0.5 hour. The reaction mixture was diluted with water (100 mL) and extracted with ethyl acetate (100 mL). The combined organic layers were washed with NaHCO₃ (30 mL×4), dried over sodium sulfate, filtered, and concentrated under reduced pressure to give a residue. The residue was purified by flash silica gel chromatography (eluent of 0˜1% ethyl acetate/petroleum ether) to give methyl 4-bromo-2,2-dimethyl-butanoate (3.4 g, 14.6 mmol, 37.1% yield, 90% purity) as a yellow oil.

¹H NMR (400 MHz, DMSO-d₆) δ=3.61 (s, 3H), 3.49-3.37 (m, 2H), 2.13-2.01 (m, 2H), 1.14 (s, 6H)

### Step B. Procedure for Preparation of methyl 4-(4-hydroxyphenyl)-2,2-dimethyl-butanoate

To a 15 mL vial equipped with a stirrer bar was added 4-bromophenol (2 g, 11.56 mmol, 1.0 equiv.), methyl 4-bromo-2,2-dimethyl-butanoate (3.14 g, 15.03 mmol, 1.3 equiv.), Ir[dF(CF₃)ppy]₂(dtbpy)(PF₆) (129.70 mg, 115.60 μmol, 0.01 equiv.), NiCl₂.dtbbpy (69.01 mg, 173.40 μmol, 0.015 equiv.), TTMSS (2.87 g, 11.56 mmol, 3.57 mL, 1.0 equiv.), and Na₂CO₃ (2.45 g, 23.12 mmol, 2.0 equiv.) in DME (20 mL). The vial was sealed and placed under nitrogen was added. The reaction was stirred and irradiated with a 10 W blue LED lamp (3 cm away), with cooling water to keep the reaction temperature at 25° C. for 14 hours. The mixture was filtered to give a filtrate, which was concentrated under reduced pressure to give a residue. The residue was purified by flash silica gel chromatography (eluent of 0˜5% ethyl acetate/petroleum ether) to give methyl 4-(4-hydroxyphenyl)-2,2-dimethyl-butanoate (1.69 g, 6.6 mmol, 57.3% yield, 87.1% purity) as a yellow oil.

MS (ESI) m/z: 221.0 [M−H]⁺

¹H NMR (400 MHz, DMSO-d₆) δ=9.11 (s, 1H), 6.94 (d, J=8.4 Hz, 2H), 6.68-6.61 (m, 2H), 3.60 (s, 3H), 2.38-2.31 (m, 2H), 1.73-1.66 (m, 2H), 1.16 (s, 6H)

### Step C. Procedure for Preparation of methyl 4-[4-(3-bromo-2-methyl-phenoxy)phenyl]-2,2-dimethyl-butanoate

A mixture of methyl 4-(4-hydroxyphenyl)-2,2-dimethyl-butanoate (1.69 g, 7.60 mmol, 1.0 equiv.), 1,3-dibromo-2-methyl-benzene (3.80 g, 15.21 mmol, 2.0 equiv.), CuI (724.00 mg, 3.80 mmol, 0.5 equiv.), Cs₂CO₃ (2.97 g, 9.12 mmol, 1.2 equiv.), and 2,2,6,6-tetramethylheptane-3,5-dione (350.26 mg, 1.90 mmol, 391.35 μL, 0.25 equiv.) in NMP (20 mL) was degassed and purged with N₂ three times, and then the mixture was stirred at 120° C. for 16 hours under N₂ atmosphere. The reaction mixture was filtered and concentrated under reduced pressure to give a residue. The residue was purified by flash silica gel chromatography (eluent of 0˜5% ethyl acetate/petroleum ether) to give methyl 4-[4-(3-bromo-2-methyl-phenoxy)phenyl]-2,2-dimethyl-butanoate (2 g, 4.6 mmol, 60.5% yield, 90.0% purity) as a green oil.

¹H NMR (400 MHz, DMSO-d₆) δ=7.41 (d, J=8.0 Hz, 1H), 7.20-7.12 (m, 3H), 6.90-6.79 (m, 3H), 3.60 (s, 3H), 2.48-2.42 (m, 2H), 2.31-2.22 (m, 3H), 1.78-1.71 (m, 2H), 1.18 (s, 6H)

### Step D. Procedure for Preparation of tert-butyl 6-[8-(1,3-benzothiazol-2-ylcarbamoyl)-3,4-dihydro-1H-isoquinolin-2-yl]-3-[3-[4-(4-methoxy-3,3-dimethyl-4-oxo-butyl)phenoxy]-2-methyl-phenyl]pyridine-2-carboxylate

Methyl 4-[4-(3-bromo-2-methyl-phenoxy)phenyl]-2,2-dimethyl-butanoate (100 mg, 255.56 μmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (140.89 mg, 230.00 μmol, 0.9 equiv.) K₂CO₃ (52.98 mg, 383.34 μmol, 1.5 equiv.), and Ad₂nBuP Pd G₃ (18.61 mg, 25.56 μmol, 0.1 equiv.) were taken up into a microwave tube in dioxane (1.5 mL) and water (0.2 mL). The sealed tube was heated at 100° C. for 2 hours under microwave. The reaction mixture was concentrated under reduced pressure to give a residue. The residue was purified by prep-TLC (SiO₂, Petroleum ether:Ethyl acetate=3:1) to give tert-butyl 6-[8-(1,3-benzothiazol-2-ylcarbamoyl)-3,4-dihydro-1H-isoquinolin-2-yl]-3-[3-[4-(4-methoxy-3,3-dimethyl-4-oxo-butyl)phenoxy]-2-methyl-phenyl]pyridine-2-carboxylate (120 mg, 130.4 μmol, 51.0% yield, 86.6% purity) as a yellow solid.

MS (ESI) m/z: 797.5 [M+H]⁺

¹H NMR (400 MHz, DMSO-d₆) δ=12.85 (s, 1H), 8.02 (d, J=8.0 Hz, 1H), 7.78 (d, J=8.0 Hz, 1H), 7.60 (d, J=7.6 Hz, 1H), 7.51 (d, J=8.8 Hz, 1H), 7.48-7.43 (m, 2H), 7.39-7.32 (m, 2H), 7.20-7.12 (m, 3H), 6.97 (d, J=8.8 Hz, 1H), 6.87-6.79 (m, 4H), 4.99 (s, 2H), 3.88 (t, J=6.0 Hz, 2H), 3.59 (s, 3H), 3.04 (t, J=5.6 Hz, 2H), 2.46-2.41 (m, 2H), 1.90 (s, 3H), 1.77-1.70 (m, 2H), 1.17 (s, 6H), 1.06 (s, 9H)

### Step E. Procedure for Preparation of 4-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]phenyl]-2,2-dimethyl-butanoic acid

To a solution of tert-butyl 6-[8-(1,3-benzothiazol-2-ylcarbamoyl)-3,4-dihydro-1H-isoquinolin-2-yl]-3-[3-[4-(4-methoxy-3,3-dimethyl-4-oxo-butyl)phenoxy]-2-methyl-phenyl]pyridine-2-carboxylate (120 mg, 150.57 μmol, 1.0 equiv.) in THE (1 mL), water (1 mL), and MeOH (1 mL) was added LiOH·H₂O (63.18 mg, 1.51 mmol, 10.0 equiv.). The mixture was stirred at 40° C. for 2 hours. The mixture was concentrated under reduced pressure to remove THF, diluted with water, and treated with HCl (1 M) until pH=5. The resulting mixture was extracted with EtOAc (30 mL×3). The combined organic layers were washed with brine (50 mL×1), dried over sodium sulfate, filtered, and concentrated under reduced pressure to give 4-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]phenyl]-2,2-dimethyl-butanoic acid (100 mg, crude) as a yellow solid.

MS (ESI) m/z: 783.3 [M+H]⁺

### Step F. Procedure for Preparation of tert-butyl 6-[8-(1,3-benzothiazol-2-ylcarbamoyl)-3,4-dihydro-1H-isoquinolin-2-yl]-3-[3-[4-[4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3,3-dimethyl-4-oxo-butyl]phenoxy]-2-methyl-phenyl]pyridine-2-carboxylate

To a solution of 4-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]phenyl]-2,2-dimethyl-butanoic acid (100 mg, 127.72 μmol, 1.0 equiv.) in DMF (1 mL) was added HATU (58.28 mg, 153.27 μmol, 1.2 equiv.), DIEA (49.52 mg, 383.17 μmol, 66.74 μL, 3.0 equiv.), and (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (73.82 mg, 166.04 μmol, 1.3 equiv.). The mixture was stirred at 20° C. for 1 hour. The mixture was added to water (10 mL), filtered to give a filter cake, which was concentrated under reduced pressure to give tert-butyl 6-[8-(1,3-benzothiazol-2-ylcarbamoyl)-3,4-dihydro-1H-isoquinolin-2-yl]-3-[3-[4-[4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3,3-dimethyl-4-oxo-butyl]phenoxy]-2-methyl-phenyl]pyridine-2-carboxylate (80 mg, crude) as an off-white solid.

MS (ESI) m/z: 605.7 [M12+H]⁺

### Step G. Procedure for Preparation of 6-[8-(1,3-benzothiazol-2-ylcarbamoyl)-3,4-dihydro-1H-isoquinolin-2-yl]-3-[3-[4-[4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3,3-dimethyl-4-oxo-butyl]phenoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[4-[4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3,3-dimethyl-4-oxo-butyl]phenoxy]-2-methyl-phenyl]pyridine-2-carboxylate (100 mg, 82.68 μmol, 1.0 equiv.) in DCM (1.5 mL) was added TFA (770.00 mg, 6.75 mmol, 0.5 mL, 81.68 equiv.). The mixture was stirred at 20° C. for 16 hours. The mixture was concentrated under reduced pressure to remove THF and diluted with DMF (2 mL). The mixture was purified by prep-HPLC to give 6-[8-(1,3-benzothiazol-2-ylcarbamoyl)-3,4-dihydro-1H-isoquinolin-2-yl]-3-[3-[4-[4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3,3-dimethyl-4-oxo-butyl]phenoxy]-2-methyl-phenyl]pyridine-2-carboxylic acid (15.7 mg, 13.1 mol, 15.9% yield, 96.8% purity) as a yellow solid.

MS (ESI) m/z: 1053.4[M+H]⁺

¹H NMR (400 MHz, DMSO-d₆) δ=13.11-12.22 (m, 1H), 8.99-8.94 (m, 1H), 8.43 (d, J=7.6 Hz, 1H), 8.02 (d, J=8.0 Hz, 1H), 7.78 (d, J=8.0 Hz, 1H), 7.62 (d, J=7.2 Hz, 1H), 7.52-7.41 (m, 5H), 7.40-7.33 (m, 4H), 7.19-7.11 (m, 3H), 6.99-6.94 (m, 1H), 6.88 (d, J=7.6 Hz, 1H), 6.85-6.77 (m, 4H), 4.99 (s, 2H), 4.94-4.87 (m, 1H), 4.57 (d, J=8.8 Hz, 1H), 4.46 (t, J=8.0 Hz, 1H), 4.31-4.26 (m, 1H), 3.95-3.89 (s, 2H), 3.62-3.58 (m, 2H), 3.05-2.99 (m, 2H), 2.45 (s, 3H), 2.42-2.37 (m, 2H), 2.06-2.00 (m, 1H), 1.90 (s, 3H), 1.81-1.70 (m, 3H), 1.36 (d, J=6.8 Hz, 3H), 1.23 (s, 1H), 1.16 (d, J=3.2 Hz, 6H), 0.98-0.93 (m, 9H)

### Example 37. Preparation of Compound 134a

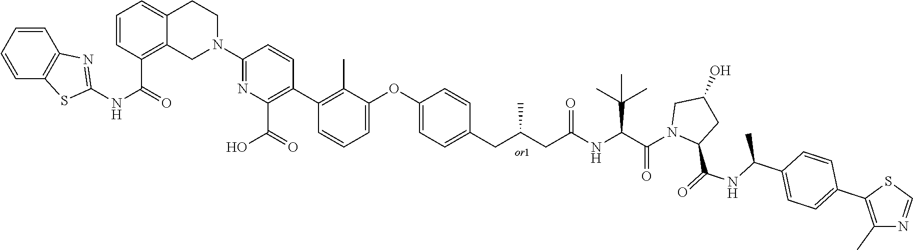 [SMILES: Cc1ncsc1-c1ccc([C@H](C)NC(=O)[C@@H]2C[C@@H](O)CN2C(=O)[C@@H](NC(=O)C[C@@H](C)Cc2ccc(Oc3cccc(-c4ccc(N5CCc6cccc(C(=O)Nc7nc8ccccc8s7)c6C5)nc4C(=O)O)c3C)cc2)C(C)(C)C)cc1]

### 6-[8-(1,3-benzothiazol-2-ylcarbamoyl)-3,4-dihydro-1H-isoquinolin-2-yl]-3-[3-[4-[(2S)-4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-methyl-4-oxo-butyl]phenoxy]-2-methyl-phenyl]pyridine-2-carboxylic acid [6-(8-(benzo[d]thiazol-2-ylcarbamoyl)-3,4-dihydroisoquinolin-2 (1H)-yl)-3-(3-(4-((S*)-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-methyl-4-oxobutyl)phenoxy)-2-methylphenyl)picolinic acid]

### Step A. Procedure for Preparation of ethyl (E)-4-(4-methoxyphenyl)-3-methyl-but-2-enoate

A mixture of ethyl 2-diethoxyphosphorylacetate (13.65 g, 60.90 mmol, 12.08 mL, 2.0 equiv.) in THE (100 mL) was degassed and purged with N₂ three times, and then the mixture was treated with LiHMDS (1 M, 91.35 mL, 3.0 equiv.) dropwise at 0° C. under N₂ atmosphere. After stirring at 0° C. for 1 hour, 1-(4-methoxyphenyl)propan-2-one (5.0 g, 30.45 mmol, 1.0 equiv.) in THE (20 mL) was added dropwise at 0° C. to the mixture, following by stirring at 25° C. for 15 hours. After completion, the reaction mixture was cooled to 0° C., and then added into a saturated NH₄Cl solution (100 mL) slowly. The solution was extracted with ethyl acetate (100 mL×3). The combined organic layers were washed with brine (200 mL), dried over anhydrous sodium sulfate, filtered, and concentrated under reduced pressure to give a residue. The residue was purified by flash silica gel chromatography (eluent of 0˜40% ethyl acetate/petroleum ether) to give ethyl (E)-4-(4-methoxyphenyl)-3-methyl-but-2-enoate (7 g, 29.8 mmol, 98.1% yield) as a colorless oil.

¹H NMR (400 MHz, DMSO-d₆) δ=7.03 (d, J=8.4 Hz, 2H), 6.80 (d, J=8.4 Hz, 2H), 5.53 (s, 1H), 3.96 (q, J=7.2 Hz, 2H), 3.65 (s, 3H), 3.30 (s, 2H), 1.95 (s, 3H), 1.09 (t, J=7.2 Hz, 3H)

### Step B. Procedure for Preparation of ethyl 4-(4-methoxyphenyl)-3-methyl-butanoate

A mixture of ethyl (E)-4-(4-methoxyphenyl)-3-methyl-but-2-enoate (3.5 g, 14.94 mmol, 1.0 equiv.), Pd/C (2 g, 1.49 mmol, 10% purity, 0.1 equiv.), and Pd(OH)₂ (2.10 g, 1.49 mmol, 10% purity, 0.1 equiv.) in MeOH (30 mL) was degassed and purged with N₂ three times, and then the mixture was stirred at 25° C. for 16 hour under H₂ (15 Psi) atmosphere. The reaction mixture was filtered and concentrated under reduced pressure to give ethyl 4-(4-methoxyphenyl)-3-methyl-butanoate (6 g, 25.3 mmol, 84.9% yield) as a gray oil.

¹H NMR (400 MHz, DMSO-d₆) δ=7.10-7.04 (m, 2H), 6.87-6.81 (m, 2H), 4.03 (q, J=7.2 Hz, 2H), 3.73-3.70 (m, 3H), 2.54-2.46 (m, 2H), 2.43-2.34 (m, 1H), 2.28-2.19 (m, 1H), 2.13-2.03 (m, 2H), 1.16 (t, J=7.2 Hz, 3H), 0.84 (d, J=6.4 Hz, 3H)

### Step C. Procedure for Preparation of ethyl 4-(4-hydroxyphenyl)-3-methyl-butanoate

To a solution of ethyl 4-(4-methoxyphenyl)-3-methyl-butanoate (1 g, 4.23 mmol, 1.0 equiv.) in DCM (10 mL) was added BBr₃ (5.30 g, 21.16 mmol, 2.04 mL, 5.0 equiv.) at −78° C. The mixture was stirred at 25° C. for 3 hours. After cooling to 0° C., the reaction was slowly quenched with MeOH (40 mL), filtered, and concentrated under reduced pressure to provide a residue. The residue was purified by column chromatography (SiO₂, petroleum ether/ethyl acetate=10/1 to 2/1) to give ethyl 4-(4-hydroxyphenyl)-3-methyl-butanoate (1 g) as a black oil.

¹H NMR (400 MHz, DMSO-d₆) δ=9.46-8.80 (m, 1H), 6.94 (d, J=8.4 Hz, 2H), 6.66 (d, J=8.4 Hz, 2H), 4.03 (q, J=7.2 Hz, 2H), 2.48-2.40 (m, 1H), 2.37-2.30 (m, 1H), 2.28-2.18 (m, 1H), 2.11-2.00 (m, 2H), 1.19-1.13 (m, 3H), 0.83 (d, J=6.0 Hz, 3H)

### Step D. Procedure for Preparation of ethyl 4-[4-(3-bromo-2-methyl-phenoxy)phenyl]-3-methyl-butanoate

A mixture of ethyl 4-(4-hydroxyphenyl)-3-methyl-butanoate (1 g, 4.50 mmol, 1.0 equiv.), 1,3-dibromo-2-methyl-benzene (1.69 g, 6.75 mmol, 1.5 equiv.), CuI (214.20 mg, 1.12 mmol, 0.25 equiv.), Cs₂CO₃ (2.20 g, 6.75 mmol, 1.2 equiv.), and 2,2,6,6-tetramethylheptane-3,5-dione (414.51 mg, 2.25 mmol, 463.14 μL, 0.25 equiv.) in NMP (10 mL) was degassed and purged with N₂ three times, and then the mixture was stirred at 120° C. for 16 hours under N₂ atmosphere. After completion, the reaction mixture was poured into water (5 mL), and then extracted with ethyl acetate (5 mL×3). The combined organic layers were washed with saturated NaHCO₃ solution (5 mL), dried over anhydrous sodium sulfate, filtered, and concentrated under reduced pressure to give a residue. The residue was purified by reverse-phase HPLC(with 0.1% HCOOH) to give ethyl 4-[4-(3-bromo-2-methyl-phenoxy)phenyl]-3-methyl-butanoate (1.5 g, 3.8 mmol, 85.2% yield) as a colorless oil.

MS (ESI) m/z 797.6 [M+H]⁺

### Step E. Procedure for Preparation of ethyl (3S)-4-[4-(3-bromo-2-methyl-phenoxy)phenyl]-3-methyl-butanoate

The stereoisomers of ethyl 4-[4-(3-bromo-2-methyl-phenoxy)phenyl]-3-methyl-butanoate were separated by SFC to give ethyl (3S)-4-[4-(3-bromo-2-methyl-phenoxy)phenyl]-3-methyl-butanoate (600 mg, 1.53 mmol, 40.00% yield) as a yellow oil and ethyl (3R)-4-[4-(3-bromo-2-methyl-phenoxy)phenyl]-3-methyl-butanoate (600 mg, 1.53 mmol, 40.00% yield) as a yellow oil.

### Step F. Procedure for Preparation of tert-butyl 6-[8-(1,3-benzothiazol-2-ylcarbamoyl)-3,4-dihydro-1H-isoquinolin-2-yl]-3-[3-[4-[(2S)-4-ethoxy-2-methyl-4-oxo-butyl]phenoxy]-2-methyl-phenyl]pyridine-2-carboxylate

A mixture of ethyl (3S)-4-[4-(3-bromo-2-methyl-phenoxy)phenyl]-3-methyl-butanoate (0.3 g, 766.68 μmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (469.63 mg, 766.68 μmol, 1.0 equiv.), K₃PO₄ (488.23 mg, 2.30 mmol, 3.0 equiv.), Ad₂nBuP Pd G₃ (111.67 mg, 153.34 mol, 0.2 equiv.), and water (0.2 mL) in dioxane (2 mL) was degassed and purged with N₂ three times, and then the mixture was stirred at 60° C. for 2 hours under N₂ atmosphere. The reaction mixture was concentrated under reduced pressure to give a residue. The crude product was purified by reverse-phase HPLC (with 0.1% HCOOH) to give tert-butyl 6-[8-(1,3-benzothiazol-2-ylcarbamoyl)-3,4-dihydro-1H-isoquinolin-2-yl]-3-[3-[4-[(2S)-4-ethoxy-2-methyl-4-oxo-butyl]phenoxy]-2-methyl-phenyl]pyridine-2-carboxylate (130 mg, 163.1 mol, 21.2% yield) as a yellow solid.

MS (ESI) m/z 797.6 [M+H]⁺

### Step G. Procedure for Preparation of (3S)-4-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]phenyl]-3-methyl-butanoic acid

To a solution of tert-butyl 6-[8-(1,3-benzothiazol-2-ylcarbamoyl)-3,4-dihydro-1H-isoquinolin-2-yl]-3-[3-[4-[(2S)-4-ethoxy-2-methyl-4-oxo-butyl]phenoxy]-2-methyl-phenyl]pyridine-2-carboxylate (130 mg, 163.12 mol, 1.0 equiv.) in THE (1 mL) was added LiOH·H₂O (1.5 M, 543.73 μL, 5.0 equiv.). The mixture was stirred at 25° C. for 16 hours. The reaction mixture was concentrated under reduced pressure to remove THE and give a water solution. The pH of above solution was adjusted to 3 with 3M HCl solution. Then the solution was filtered to give (3S)-4-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]phenyl]-3-methyl-butanoic acid (120 mg, 156.0 mol, 95.6% yield) as a white solid.

MS (ESI) m/z 769.5 [M+H]⁺

### Step H. Procedure for Preparation of tert-butyl 6-[8-(1,3-benzothiazol-2-ylcarbamoyl)-3,4-dihydro-1H-isoquinolin-2-yl]-3-[3-[4-[(2S)-4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-methyl-4-oxo-butyl]phenoxy]-2-methyl-phenyl]pyridine-2-carboxylate

To a solution of (3S)-4-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]phenyl]-3-methyl-butanoic acid (120 mg, 156.06 mol, 1.0 equiv.) in Py (1.5 mL) was added EDCI (149.59 mg, 780.32 mol, 5.0 equiv.), (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (76.32 mg, 171.67 mol, 1.1 equiv.). The mixture was stirred at 40° C. for 16 hours. The mixture was poured into water (5 mL), then filtered to give tert-butyl 6-[8-(1,3-benzothiazol-2-ylcarbamoyl)-3,4-dihydro-1H-isoquinolin-2-yl]-3-[3-[4-[(2S)-4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-methyl-4-oxo-butyl]phenoxy]-2-methyl-phenyl]pyridine-2-carboxylate (100 mg, 83.6 mol, 53.6% yield) as a yellow solid.

### Step I. Procedure for Preparation of 6-[8-(1,3-benzothiazol-2-ylcarbamoyl)-3,4-dihydro-1H-isoquinolin-2-yl]-3-[3-[4-[(2S)-4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-methyl-4-oxo-butyl]phenoxy]-2-methyl-phenyl]pyridine-2-carboxylic acid [6-(8-(benzo[d]thiazol-2-ylcarbamoyl)-3,4-dihydroisoquinolin-2 (1H)-yl)-3-(3-(4-((S*)-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-methyl-4-oxobutyl)phenoxy)-2-methylphenyl)picolinic acid]

To a solution of tert-butyl 6-[8-(1,3-benzothiazol-2-ylcarbamoyl)-3,4-dihydro-1H-isoquinolin-2-yl]-3-[3-[4-[(2S)-4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-methyl-4-oxo-butyl]phenoxy]-2-methyl-phenyl]pyridine-2-carboxylate (100 mg, 83.65 mol, 1 equiv.) in DCM (1 mL) was added TFA (461.99 mg, 4.05 mmol, 299.99 μL, 48.44 equiv.). The mixture was stirred at 40° C. for 16 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[4-[(2S)-4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-methyl-4-oxo-butyl]phenoxy]-2-methyl-phenyl]pyridine-2-carboxylic acid [6-(8-(benzo[d]thiazol-2-ylcarbamoyl)-3,4-dihydroisoquinolin-2 (1H)-yl)-3-(3-(4-((S*)-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-methyl-4-oxobutyl)phenoxy)-2-methylphenyl)picolinic acid](16.0 mg, 13.4 mol, 16.1% yield, 95.6% purity) as an off-white solid.

MS (ESI) m/z 1139.9 [M+H]⁺

¹H NMR (400 MHz, DMSO-d₆) δ=8.98 (s, 1H), 8.40-8.32 (m, 1H), 8.03-7.99 (m, 1H), 7.86-7.82 (m, 1H), 7.78 (d, J=7.2 Hz, 1H), 7.69-7.56 (m, 1H), 7.46-7.31 (m, 9H), 7.17-7.08 (m, 3H), 6.92-6.77 (m, 5H), 5.16-4.96 (m, 2H), 4.93-4.87 (m, 1H), 4.57-4.50 (m, 1H), 4.40 (t, J=8.4 Hz, 1H), 4.27 (s, 1H), 3.99-3.76 (m, 2H), 3.61 (s, 2H), 3.10-2.95 (m, 4H), 2.45 (s, 3H), 2.13-1.98 (m, 5H), 1.91 (s, 3H), 1.80-1.74 (m, 1H), 1.36 (d, J=7.2 Hz, 3H), 0.95 (s, 9H), 0.80 (d, J=2.0 Hz, 3H)

### Example 38. Preparation of Compound 134b

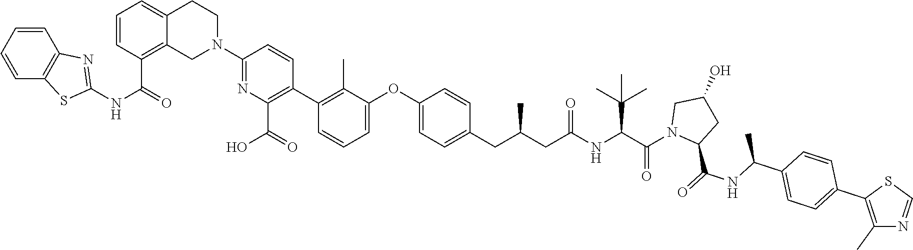 [SMILES: Cc1ncsc1-c1ccc([C@H](C)NC(=O)[C@@H]2C[C@@H](O)CN2C(=O)[C@@H](NC(=O)C[C@H](C)Cc2ccc(Oc3cccc(-c4ccc(N5CCc6cccc(C(=O)Nc7nc8ccccc8s7)c6C5)nc4C(=O)O)c3C)cc2)C(C)(C)C)cc1]

### 6-[8-(1,3-benzothiazol-2-ylcarbamoyl)-3,4-dihydro-1H-isoquinolin-2-yl]-3-[3-[4-[(2R)-4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-methyl-4-oxo-butyl]phenoxy]-2-methyl-phenyl]pyridine-2-carboxylic acid [6-(8-(benzo[d]thiazol-2-ylcarbamoyl)-3,4-dihydroisoquinolin-2 (1H)-yl)-3-(3-(4-((R*)-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-methyl-4-oxobutyl)phenoxy)-2-methylphenyl)picolinic acid]

### Step A. Procedure for Preparation of tert-butyl 6-[8-(1,3-benzothiazol-2-ylcarbamoyl)-3,4-dihydro-1H-isoquinolin-2-yl]-3-[3-[4-[(2R)-4-ethoxy-2-methyl-4-oxo-butyl]phenoxy]-2-methyl-phenyl]pyridine-2-carboxylate

A mixture of ethyl (3R)-4-[4-(3-bromo-2-methyl-phenoxy)phenyl]-3-methyl-butanoate (0.3 g, 766.68 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (469.63 mg, 766.68 μmol, 1 equiv.), K₃PO₄ (488.23 mg, 2.30 mmol, 3 equiv.), Ad₂nBuP Pd G₃ (111.67 mg, 153.34 mol, 0.2 equiv.), and water (0.2 mL) in dioxane (2 mL) was degassed and purged with N₂ three times. The mixture was then stirred at 60° C. for 16 hours under N₂ atmosphere. The reaction mixture was concentrated under reduced pressure to give a residue. The crude product was purified by reverse-phase HPLC (with 0.1% formic acid) to give tert-butyl 6-[8-(1,3-benzothiazol-2-ylcarbamoyl)-3,4-dihydro-1H-isoquinolin-2-yl]-3-[3-[4-[(2R)-4-ethoxy-2-methyl-4-oxo-butyl]phenoxy]-2-methyl-phenyl]pyridine-2-carboxylate (250 mg, 313.6 mol, 40.9% yield) as a yellow solid.

MS (ESI) m/z 797.6 [M+H]⁺

### Step B. Procedure for Preparation of (3R)-4-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]phenyl]-3-methyl-butanoic acid

To a solution of tert-butyl 6-[8-(1,3-benzothiazol-2-ylcarbamoyl)-3,4-dihydro-1H-isoquinolin-2-yl]-3-[3-[4-[(2R)-4-ethoxy-2-methyl-4-oxo-butyl]phenoxy]-2-methyl-phenyl]pyridine-2-carboxylate (160 mg, 200.76 mol, 1 equiv.) in THE (1 mL) was added LiOH·H₂O (1.5 M, 669.20 μL, 5 equiv.). The mixture was stirred at 25° C. for 36 hours. The reaction mixture was concentrated under reduced pressure to give a residue. The residue was diluted with water to give a solution. The pH of the solution was adjusted to 3 with 3M HCl solution. Then the resulting mixture was filtered to give (3R)-4-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]phenyl]-3-methyl-butanoic acid (180 mg, crude) as a yellow solid.

MS (ESI) m/z 769.5 [M+H]⁺

### Step C. Procedure for Preparation of tert-butyl 6-[8-(1,3-benzothiazol-2-ylcarbamoyl)-3,4-dihydro-1H-isoquinolin-2-yl]-3-[3-[4-[(2R)-4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-methyl-4-oxo-butyl]phenoxy]-2-methyl-phenyl]pyridine-2-carboxylate

To a solution of (3R)-4-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]phenyl]-3-methyl-butanoic acid (180 mg, 234.10 mol, 1.0 equiv.) in pyridine (2 mL) was added EDCI (89.75 mg, 468.19 mol, 2.0 equiv.) and (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (208.15 mg, 468.19 mol, 2.0 equiv.). The mixture was stirred at 25° C. for 1 hour. The mixture was poured into water (20 mL) and filtered to give tert-butyl 6-[8-(1,3-benzothiazol-2-ylcarbamoyl)-3,4-dihydro-1H-isoquinolin-2-yl]-3-[3-[4-[(2R)-4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-methyl-4-oxo-butyl]phenoxy]-2-methyl-phenyl]pyridine-2-carboxylate (150 mg, 125.4 mol, 53.6% yield) as a yellow solid.

### Step D. Procedure for Preparation of 6-[8-(1,3-benzothiazol-2-ylcarbamoyl)-3,4-dihydro-1H-isoquinolin-2-yl]-3-[3-[4-[(2R)-4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-methyl-4-oxo-butyl]phenoxy]-2-methyl-phenyl]pyridine-2-carboxylic acid [6-(8-(benzo[d]thiazol-2-ylcarbamoyl)-3,4-dihydroisoquinolin-2 (1H)-yl)-3-(3-(4-((R*)-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-methyl-4-oxobutyl)phenoxy)-2-methylphenyl)picolinic acid]

To a solution of tert-butyl 6-[8-(1,3-benzothiazol-2-ylcarbamoyl)-3,4-dihydro-1H-isoquinolin-2-yl]-3-[3-[4-[(2R)-4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-methyl-4-oxo-butyl]phenoxy]-2-methyl-phenyl]pyridine-2-carboxylate (150 mg, 125.47 mol, 1.0 equiv.) in DCM (0.5 mL) was added TFA (1.15 g, 10.13 mmol, 749.98 μL, 80.73 equiv.). The mixture was stirred at 30° C. for 16 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[4-[(2R)-4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-methyl-4-oxo-butyl]phenoxy]-2-methyl-phenyl]pyridine-2-carboxylic acid [6-(8-(benzo[d]thiazol-2-ylcarbamoyl)-3,4-dihydroisoquinolin-2 (1H)-yl)-3-(3-(4-((R*)-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-methyl-4-oxobutyl)phenoxy)-2-methylphenyl)picolinic acid](20.0 mg, 15.5 mol, 12.4% yield, 88.4% purity) was obtained as a yellow solid.

MS (ESI) m/z 1139.3 [M+H]⁺

¹H NMR (400 MHz, DMSO-d₆) δ=9.01-8.95 (m, 1H), 8.42-8.33 (m, 1H), 8.03 (d, J=7.6 Hz, 1H), 7.85 (d, J=9.2 Hz, 1H), 7.79 (d, J=8.0 Hz, 1H), 7.63 (d, J=7.6 Hz, 1H), 7.53-7.34 (m, 9H), 7.20-7.10 (m, 3H), 7.00-6.87 (m, 2H), 6.87-6.77 (m, 3H), 5.03-4.97 (m, 2H), 4.95-4.88 (m, 1H), 4.54 (d, J=9.2 Hz, 1H), 4.43 (t, J=8.0 Hz, 1H), 4.28 (s, 1H), 3.93 (t, J=5.6 Hz, 2H), 3.62 (s, 2H), 3.03 (t, J=5.6 Hz, 2H), 2.65-2.53 (m, 2H), 2.46 (s, 3H), 2.34-2.20 (m, 2H), 2.12-2.00 (m, 3H), 1.92 (s, 3H), 1.84-1.75 (m, 1H), 1.38 (d, J=6.8 Hz, 3H), 0.95 (s, 9H), 0.78 (d, J=6.0 Hz, 3H)

### Example 39. Preparation of Compound 135a

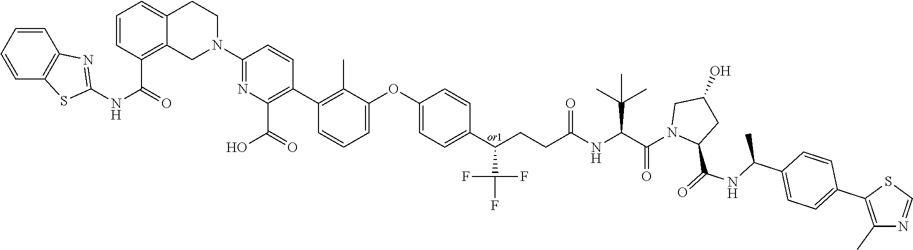 [SMILES: Cc1ncsc1-c1ccc([C@H](C)NC(=O)[C@@H]2C[C@@H](O)CN2C(=O)[C@@H](NC(=O)CC[C@@H](c2ccc(Oc3cccc(-c4ccc(N5CCc6cccc(C(=O)Nc7nc8ccccc8s7)c6C5)nc4C(=O)O)c3C)cc2)C(F)(F)F)C(C)(C)C)cc1]

### 6-(8-(benzo[d]thiazol-2-ylcarbamoyl)-3,4-dihydroisoquinolin-2 (1H)-yl)-3-(2-methyl-3-(4-((S)-1,1,1-trifluoro-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)phenyl)picolinic acid [6-(8-(benzo[d]thiazol-2-ylcarbamoyl)-3,4-dihydroisoquinolin-2 (1H)-yl)-3-(2-methyl-3-(4-((S*)-1,1,1-trifluoro-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)phenyl)picolinic acid]

### Step A. Procedure for Preparation of (Z)-1-(5-(benzyloxy)-1,1,1-trifluoropent-2-en-2-yl)-4-methoxybenzene

To a solution of (3-(benzyloxy)propyl)triphenyl-14-phosphane bromine (10.88 g, 26.45 mmol, 1.2 equiv.) in THF (50 mL) was added dropwise LiHMDS (1 M, 33.06 mL, 1.5 equiv.) at −70° C. over 15 min. After addition, the mixture was stirred at this temperature for 1 hour, and then 2,2,2-trifluoro-1-(4-methoxyphenyl)ethan-1-one (4.5 g, 22.04 mmol, 1 equiv.) in THF (2 mL) was added dropwise at −70° C. over 15 min. The resulting mixture was stirred at 20° C. for 16 hours. The reaction mixture was quenched by addition saturated NH₄Cl (20 mL) and then extracted with EtOAc (10 mL×2). The combined organic layers were washed with brine (5 mL×2), dried over sodium sulfate, filtered, and concentrated under reduced pressure to give a residue. The residue was purified by flash silica gel chromatography (eluent of 0˜10% ethyl acetate/petroleum ether) to give (Z)-1-(5-(benzyloxy)-1,1,1-trifluoropent-2-en-2-yl)-4-methoxybenzene (5.5 g) as a colorless oil.

¹H NMR (400 MHz, DMSO-d₆) δ=7.34 (s, 1H), 7.29 (s, 1H), 7.31-7.28 (m, 2H), 7.21-7.15 (m, 2H), 7.03-6.97 (m, 2H), 6.53 (t, J=7.2 Hz, 1H), 4.43 (s, 2H), 3.78 (s, 3H), 3.54-3.46 (m, 2H), 2.27 (d, J=5.2 Hz, 2H)

### Step B. Procedure for Preparation of 5,5,5-trifluoro-4-(4-methoxyphenyl)pentan-1-ol

To a solution of (Z)-1-(5-(benzyloxy)-1,1,1-trifluoropent-2-en-2-yl)-4-methoxybenzene (5.5 g, 16.35 mmol, 1 equiv.) in CH₃COOH (2 mL) and EtOH (60 mL) was added Pd/C (2 g, 32.70 mmol, 10% purity, 2 equiv.) and Pd(OH)₂ (4.59 g, 32.70 mmol, 2 equiv.). The mixture was stirred at 60° C. for 24 hours under H₂ atmosphere at 50 Psi. The reaction mixture was filtered and concentrated under reduced pressure to give a residue. The residue was purified by flash silica gel chromatography (eluent of 0˜20% ethyl acetate/petroleum ether). 5,5,5-Trifluoro-4-(4-methoxyphenyl)pentan-1-ol (3.99 g) was obtained as a colorless oil.

¹H NMR (400 MHz, DMSO-d₆) δ=7.27 (d, J=8.6 Hz, 2H), 6.98-6.91 (m, 2H), 3.75 (s, 3H), 3.59-3.45 (m, 1H), 1.99-1.90 (m, 1H), 1.87-1.76 (m, 1H), 1.34-1.15 (m, 2H)

### Step C. Procedure for Preparation of 4-(1,1,1-trifluoro-5-hydroxypentan-2-yl)phenol

To a solution of 5,5,5-trifluoro-4-(4-methoxyphenyl)pentan-1-ol (1.8 g, 7.25 mmol, 1 equiv.) in DCM (10 mL) was added BBr₃ (9.08 g, 36.26 mmol, 3.49 mL, 5 equiv.) at −78° C. The mixture was stirred at 20° C. for 2 hours. After cooling to 0° C., the reaction was slowly quenched with MeOH (40 mL), filtered, and concentrated under reduced pressure to obtain a residue. The residue was purified by flash silica gel chromatography (eluent of 0˜30% ethyl acetate/petroleum ether) to give 4-(1,1,1-trifluoro-5-hydroxypentan-2-yl)phenol (966 mg) as a colorless oil

¹H NMR (400 MHz, DMSO-d₆) δ=9.4 (s, 1H), 7.2 (d, J=8.4 Hz, 2H), 6.75 (d, J=8.4 Hz, 2H), 4.47-4.36 (m, 1H), 3.52-3.35 (m, 2H), 1.97-1.86 (m, 1H), 1.85-1.72 (m, 1H), 1.34-1.19 (m, 2H)

### Step D. Procedure for Preparation of 4-(4-(3-bromo-2-methylphenoxy)phenyl)-5,5,5-trifluoropentan-1-ol

A mixture of 1,3-dibromo-2-methyl-benzene (640.26 mg, 2.56 mmol, 1.2 equiv.), 4-(1,1,1-trifluoro-5-hydroxypentan-2-yl)phenol (500 mg, 2.13 mmol, 1 equiv.), Cs₂CO₃ (834.67 mg, 2.56 mmol, 1.2 equiv.), and 2,2,6,6-tetramethylheptane-3,5-dione (98.35 mg, 533.70 μmol, 109.89 μL, 0.25 equiv.) in NMP (5 mL) was degassed and purged with N₂ three times. CuI (203.29 mg, 1.07 mmol, 0.5 equiv.) was added to the mixture, which was stirred at 120° C. for 16 hour under N₂ atmosphere. The reaction mixture was quenched by the addition of water (3 mL) at 25° C. and extracted with ethyl acetate (5 mL×2). The combined organic layers were concentrated under reduced pressure to give a residue. The residue was purified by flash silica gel chromatography (eluent of 0˜30% ethyl acetate/petroleum ether) to give 4-(4-(3-bromo-2-methylphenoxy)phenyl)-5,5,5-trifluoropentan-1-ol (530 mg, 1.2 mmol, 58.4% yield, 95% purity) as a white solid

¹H NMR (400 MHz, CDCl₃) δ=7.44-7.27 (m, 1H), 7.18-7.05 (m, 2H), 7.01-6.91 (m, 1H), 6.80 (d, J=8.6 Hz, 3H), 3.60-3.53 (m, 2H), 3.26-3.08 (m, 1H), 2.30-2.22 (m, 3H), 2.08-1.99 (m, 1H), 1.92-1.77 (m, 1H), 1.41-1.36 (m, 2H).

### Step E. Procedure for Preparation of 4-(4-(3-bromo-2-methylphenoxy)phenyl)-5,5,5-trifluoropentanal

A mixture of DMSO (399.94 mg, 5.12 mmol, 399.94 μL, 4 equiv.) in DCM (3 mL) was degassed and purged with N₂ three times, and then the oxalyl dichloride (324.84 mg, 2.56 mmol, 224.03 μL, 2 equiv.) was added, and the resulting mixture was stirred at −65° C. for 1 hour under N₂ atmosphere. A mixture of 4-(4-(3-bromo-2-methylphenoxy)phenyl)-5,5,5-trifluoropentan-1-ol (516 mg, 1.28 mmol, 1 equiv.) in DCM (8 mL) was added to the mixture, which was stirred at −65° C. for 1.5 hours under N₂ atmosphere. TEA (776.92 mg, 7.68 mmol, 1.07 mL, 6 equiv.) was added to the mixture, and the mixture was stirred at −65° C. for 0.5 hour under N₂ atmosphere. The reaction mixture was then diluted with water (20 mL) and extracted with DCM (10 mL×2). The combined organic layers were concentrated under reduced pressure to give a residue. The residue was purified by flash silica gel chromatography (eluent of 0-30% ethyl acetate/petroleum ether). 4-(4-(3-Bromo-2-methylphenoxy)phenyl)-5,5,5-trifluoropentanal (370 mg, 922.1 μmol, 72.0% yield) was obtained as a colorless oil.

### Step F. Procedure for Preparation of (S)-4-(4-(3-bromo-2-methylphenoxy)phenyl)-5,5,5-trifluoropentanoic acid

To a solution of 4-(4-(3-bromo-2-methylphenoxy)phenyl)-5,5,5-trifluoropentanal (350 mg, 872.35 μmol, 1 equiv.) in t-BuOH (3.2 mL) and THE (1.6 mL) and water (0.8 mL) was added sodium chlorite (236.69 mg, 2.62 mmol, 3 equiv.), 2-methylbut-2-ene (367.08 mg, 5.23 mmol, 554.50 μL, 6 equiv.) and sodium dihydrogen phosphate (313.98 mg, 2.62 mmol, 3 equiv.). The mixture was stirred at 25° C. for 12 hours. The reaction mixture was concentrated under reduced pressure to give a residue. The residue was purified by flash silica gel chromatography. The stereoisomers of the purified product were separated by SFC. (S)-4-(4-(3-Bromo-2-methylphenoxy)phenyl)-5,5,5-trifluoropentanoic acid (130 mg) and (4R)-4-[4-(3-bromo-2-methyl-phenoxy)phenyl]-5,5,5-trifluoro-pentanoic acid (65 mg) were obtained as light yellow oils.

¹H NMR (400 MHz, CDCl₃) δ=7.43-7.36 (m, 1H), 7.22 (d, J=8.4 Hz, 2H), 7.09-7.00 (m, 1H), 6.91-6.87 (m, 3H), 3.43-3.27 (m, 1H), 2.45-2.35 (m, 3H), 2.33 (s, 3H), 2.30-2.27 (m, 1H), 2.19-2.09 (m, 2H)

### Step G. Procedure for Preparation of ethyl (S)-4-(4-(3-bromo-2-methylphenoxy)phenyl)-5,5,5-trifluoropentanoate

To a solution of (S)-4-(4-(3-bromo-2-methylphenoxy)phenyl)-5,5,5-trifluoropentanoic acid (130.00 mg, 311.59 μmol, 1 equiv.) in EtOH (0.1 mL) was added SOCl₂ (66.73 mg, 560.86 μmol, 40.69 μL, 1.8 equiv.) at 0° C. The mixture was stirred at 70° C. for 2.5 hours. The reaction mixture was concentrated under reduced pressure to give ethyl (S)-4-(4-(3-bromo-2-methylphenoxy)phenyl)-5,5,5-trifluoropentanoate (135 mg, crude) as a colorless oil, which was used in the next step without further purification.

### Step H. Procedure for Preparation of tert-butyl (S)-6-(8-(benzo[d]thiazol-2-ylcarbamoyl)-3,4-dihydroisoquinolin-2 (1H)-yl)-3-(3-(4-(5-ethoxy-1,1,1-trifluoro-5-oxopentan-2-yl)phenoxy)-2-methylphenyl)picolinate

Tert-butyl 6-(8-(benzo[d]thiazol-2-ylcarbamoyl)-3,4-dihydroisoquinolin-2 (1H)-yl)-3-(4,4,5,5-tetramethyl-1,3,2-dioxaborolan-2-yl)picolinate (33.02 mg, 53.90 μmol, 1.2 equiv.), K₂CO₃ (1.5 M, 29.94 μL, 1 equiv.), and ethyl (S)-4-(4-(3-bromo-2-methylphenoxy)phenyl)-5,5,5-trifluoropentanoate (20.00 mg, 44.92 μmol, 1 equiv.) were taken up into a microwave tube in dioxane (0.8 mL). [2-(2-aminophenyl)phenyl]palladium(1+);bis(1-adamantyl)-butyl-phosphane;methanesulfonate (6.54 mg, 8.98 μmol, 0.2 equiv.) was added to the above mixture. The sealed tube was heated at 100° C. for 1 hour under microwave. The reaction mixture was concentrated under reduced pressure to give a residue. The residue was purified by flash silica gel chromatography (eluent of 0˜50% ethyl acetate/petroleum ether) to give tert-butyl (S)-6-(8-(benzo[d]thiazol-2-ylcarbamoyl)-3,4-dihydroisoquinolin-2 (1H)-yl)-3-(3-(4-(5-ethoxy-1,1,1-trifluoro-5-oxopentan-2-yl)phenoxy)-2-methylphenyl)picolinate (188 mg) as a white solid.

MS (ESI) m/z: 851.4[M+H]⁺.

### Step I. Procedure for Preparation of (S)-4-(4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)phenyl)-5,5,5-trifluoropentanoic acid

To a solution of tert-butyl (S)-6-(8-(benzo[d]thiazol-2-ylcarbamoyl)-3,4-dihydroisoquinolin-2 (1H)-yl)-3-(3-(4-(5-ethoxy-1,1,1-trifluoro-5-oxopentan-2-yl)phenoxy)-2-methylphenyl)picolinate (188 mg, 220.93 μmol, 1 equiv.) in THE (3 mL) and water (1.5 mL) was added LiOH·H₂O (27.81 mg, 662.80 μmol, 3 equiv.). The mixture was stirred at 25° C. for 5 hours. The pH of the reaction mixture was adjusted to 5-7 by 1M HCl, and then the mixture was diluted with water (5 mL) and extracted with DCM (5 mL×2). The combined organic layers was concentrated under reduced pressure to give (S)-4-(4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)phenyl)-5,5,5-trifluoropentanoic acid (170 mg, crude) as a white solid, which was carried in the next step without further purification.

MS (ESI) m/z: 823.4[M+H]⁺.

### Step J. Procedure for Preparation of tert-butyl 6-(8-(benzo[d]thiazol-2-ylcarbamoyl)-3,4-dihydroisoquinolin-2 (1H)-yl)-3-(2-methyl-3-(4-((S)-1,1,1-trifluoro-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)phenyl)picolinate

To a solution of (S)-4-(4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)phenyl)-5,5,5-trifluoropentanoic acid (50.00 mg, 60.76 mol, 1 equiv.) in DMF (1 mL) was added HATU (69.31 mg, 182.28 mol, 3 equiv.) and DIEA (47.12 mg, 364.57 mol, 63.50 μL, 3 equiv.). The mixture was stirred at 25° C. for 0.5 hour. Then (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (32.42 mg, 72.91 mol, 1.2 equiv.) was added to the mixture, which was stirred for 16 hours. The reaction mixture was quenched by addition brine (5 mL) at 25° C. and then diluted and extracted with EtOAc (10 mL). The combined organic layers were washed with brine (5 mL) and concentrated under reduced pressure to give a residue. The residue was purified by prep-TLC (SiO₂, DCM:MeOH=10:1) to give tert-butyl 6-(8-(benzo[d]thiazol-2-ylcarbamoyl)-3,4-dihydroisoquinolin-2 (1H)-yl)-3-(2-methyl-3-(4-((S)-1,1,1-trifluoro-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)phenyl)picolinate (40 mg) as a white solid.

MS (ESI) m/z: 625.9[M/2+H]⁺.

### Step K. Procedure for Preparation of 6-(8-(benzo[d]thiazol-2-ylcarbamoyl)-3,4-dihydroisoquinolin-2 (1H)-yl)-3-(2-methyl-3-(4-((S)-1,1,1-trifluoro-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)phenyl)picolinic acid [6-(8-(benzo[d]thiazol-2-ylcarbamoyl)-3,4-dihydroisoquinolin-2 (1H)-yl)-3-(2-methyl-3-(4-((S*)-1,1,1-trifluoro-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)phenyl)picolinic acid]

To a solution of tert-butyl 6-(8-(benzo[d]thiazol-2-ylcarbamoyl)-3,4-dihydroisoquinolin-2 (1H)-yl)-3-(2-methyl-3-(4-((S)-1,1,1-trifluoro-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)phenyl)picolinate (40 mg, 32.01 mol, 1 equiv.) in DCM (1 mL) was added TFA (3.65 mg, 32.01 mol, 2.38 μL, 1 equiv.). The mixture was stirred at 25° C. for 1 hour. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2 (1H)-yl)-3-(2-methyl-3-(4-((S)-1,1,1-trifluoro-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)phenyl)picolinic acid [6-(8-(benzo[d]thiazol-2-ylcarbamoyl)-3,4-dihydroisoquinolin-2 (1H)-yl)-3-(2-methyl-3-(4-((S*)-1,1,1-trifluoro-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)phenyl)picolinic acid](18.1 mg, 14.0 mol, 43.7% yield, 92.2% purity) as a yellow solid

MS (ESI) m/z: 597.4 [M/2+H]

¹H NMR (400 MHz, DMSO-d₆) δ=12.63 (s, 1H), 9.02-8.93 (m, 1H), 8.36 (d, J=8.0 Hz, 1H), 8.03 (d, J=8.0 Hz, 1H), 7.93-7.86 (m, 1H), 7.83-7.74 (m, 1H), 7.63 (d, J=7.6 Hz, 1H), 7.53 (d, J=8.6 Hz, 1H), 7.50-7.47 (m, 1H), 7.46-7.41 (m, 3H), 7.41-7.34 (m, 4H), 7.32 (d, J=8.4 Hz, 2H), 7.23-7.18 (m, 1H), 6.99 (d, J=8.0 Hz, 1H), 6.96-6.85 (m, 4H), 5.00 (s, 2H), 4.91 (t, J=7.2 Hz, 1H), 4.49 (d, J=9.2 Hz, 1H), 4.45-4.38 (m, 1H), 4.28 (s, 1H), 3.93 (t, J=5.6 Hz, 2H), 3.60 (s, 2H), 3.04 (t, J=5.6 Hz, 2H), 2.49-2.42 (m, 5H), 2.15 (d, J=9.6 Hz, 2H), 2.03 (dd, J=4.0, 12.2 Hz, 3H), 1.92 (s, 3H), 1.84-1.74 (m, 1H), 1.37 (d, J=7.0 Hz, 2H), 0.95-0.85 (m, 9H)

### Example 40. Preparation of Compound 135b

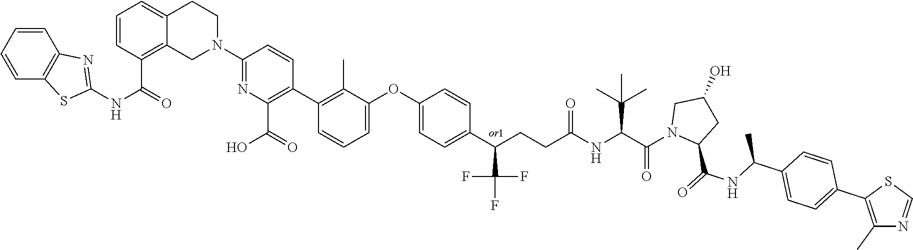 [SMILES: Cc1ncsc1-c1ccc([C@H](C)NC(=O)[C@@H]2C[C@@H](O)CN2C(=O)[C@@H](NC(=O)CC[C@H](c2ccc(Oc3cccc(-c4ccc(N5CCc6cccc(C(=O)Nc7nc8ccccc8s7)c6C5)nc4C(=O)O)c3C)cc2)C(F)(F)F)C(C)(C)C)cc1]

### 6-(8-(benzo[d]thiazol-2-ylcarbamoyl)-3,4-dihydroisoquinolin-2 (1H)-yl)-3-(2-methyl-3-(4-((R)-1,1,1-trifluoro-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)phenyl)picolinic acid [6-(8-(benzo[d]thiazol-2-ylcarbamoyl)-3,4-dihydroisoquinolin-2 (1H)-yl)-3-(2-methyl-3-(4-((R*)-1,1,1-trifluoro-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)phenyl)picolinic acid]

### Step A. Procedure for Preparation of ethyl (R)-4-(4-(3-bromo-2-methylphenoxy)phenyl)-5,5,5-trifluoropentanoate

To a solution of (R)-4-(4-(3-bromo-2-methylphenoxy)phenyl)-5,5,5-trifluoropentanoic acid (65.00 mg, 155.79 μmol, 1 equiv.) in EtOH (0.1 mL) was added SOCl₂ (33.36 mg, 280.43 μmol, 20.34 μL, 1.8 equiv.) at 0° C. The mixture was stirred at 70° C. for 2.5 hours. The reaction mixture was then concentrated under reduced pressure to give ethyl (R)-4-(4-(3-bromo-2-methylphenoxy)phenyl)-5,5,5-trifluoropentanoate (68 mg, crude) as a colorless oil, which was carried into the next step without further purification.

### Step B. Procedure for Preparation of tert-butyl (R)-6-(8-(benzo[d]thiazol-2-ylcarbamoyl)-3,4-dihydroisoquinolin-2 (1H)-yl)-3-(3-(4-(5-ethoxy-1,1,1-trifluoro-5-oxopentan-2-yl)phenoxy)-2-methylphenyl)picolinate

Tert-butyl 6-(8-(benzo[d]thiazol-2-ylcarbamoyl)-3,4-dihydroisoquinolin-2 (1H)-yl)-3-(4,4,5,5-tetramethyl-1,3,2-dioxaborolan-2-yl)picolinate (107.30 mg, 175.17 μmol, 1.2 equiv.), ethyl (R)-4-(4-(3-bromo-2-methylphenoxy)phenyl)-5,5,5-trifluoropentanoate (65.00 mg, 145.98 μmol, 1 equiv.), and K₂CO₃ (1.5 M, 97.32 μL, 1 equiv.) were taken up into a microwave tube in dioxane (2 mL). [2-(2-Aminophenyl)phenyl]palladium(1′); bis(1-adamantyl)-butyl-phosphane;methanesulfonate (21.26 mg, 29.20 μmol, 0.2 equiv.) was added to the above mixture. The sealed tube was heated at 100° C. for 1 hour under microwave. The reaction mixture was concentrated under reduced pressure to give a residue. The residue was purified by flash silica gel chromatography (eluent of 0˜50% ethyl acetate/petroleum ether) to give tert-butyl (R)-6-(8-(benzo[d]thiazol-2-ylcarbamoyl)-3,4-dihydroisoquinolin-2 (1H)-yl)-3-(3-(4-(5-ethoxy-1,1,1-trifluoro-5-oxopentan-2-yl)phenoxy)-2-methylphenyl)picolinate (102 mg) as a white oil.

MS (ESI) m/z: 851.4[M+H]⁺.

### Step C. Procedure for Preparation of (R)-4-(4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)phenyl)-5,5,5-trifluoropentanoic acid

To a solution of tert-butyl (R)-6-(8-(benzo[d]thiazol-2-ylcarbamoyl)-3,4-dihydroisoquinolin-2 (1H)-yl)-3-(3-(4-(5-ethoxy-1,1,1-trifluoro-5-oxopentan-2-yl)phenoxy)-2-methylphenyl)picolinate (100 mg, 117.52 μmol, 1 equiv.) in THE (3 mL) and water (1.5 mL) was added LiOH·H₂O (4.93 mg, 117.52 μmol, 1 equiv.). The mixture was stirred at 25° C. for 5 hours. The pH of the reaction mixture was adjusted to 5-7 by 1M HCl, Extracted with DCM 10 mL (5 mL×2). The combined organic layers concentrated under reduced pressure to give the crude product (R)-4-(4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)phenyl)-5,5,5-trifluoropentanoic acid (90 mg, crude) as a white solid, which was carried into the next step without further purification.

MS (ESI) m/z: 823.4[M+H]⁺.

### Step D. Procedure for Preparation of tert-butyl 6-(8-(benzo[d]thiazol-2-ylcarbamoyl)-3,4-dihydroisoquinolin-2 (1H)-yl)-3-(2-methyl-3-(4-((R)-1,1,1-trifluoro-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)phenyl)picolinate

To a solution of (R)-4-(4-(3-(6-(8-(benzo[d]thiazol-2-ylcarbamoyl)-3,4-dihydroisoquinolin-2 (1H)-yl)-2-(tert-butoxycarbonyl)pyridin-3-yl)-2-methylphenoxy)phenyl)-5,5,5-trifluoropentanoic acid (65 mg, 78.99 mol, 1 equiv.) in DMF (1 mL) was added HATU (90.10 mg, 236.97 mol, 3 equiv.) and DIEA (61.25 mg, 473.94 mol, 82.55 μL, 6 equiv.). The mixture was stirred at 25° C. for 0.5 hour. Then (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (42.14 mg, 94.79 mol, 1.2 equiv.) was added to the mixture, which was stirred for 16 hours. The reaction mixture was quenched by the addition of brine (5 mL) at 25° C., and then diluted and extracted with EtOAc (10 mL). The combined organic layers were washed with brine (5 mL), diluted and extracted with EtOAc (10 mL), and concentrated under reduced pressure to give a residue. The residue was purified by prep-TLC (SiO₂, DCM:MeOH=10:1) to give tert-butyl 6-(8-(benzo[d]thiazol-2-ylcarbamoyl)-3,4-dihydroisoquinolin-2 (1H)-yl)-3-(2-methyl-3-(4-((R)-1,1,1-trifluoro-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)phenyl)picolinate (50 mg) as a white solid.

MS (ESI) m/z: 625.8[M/2+H]⁺.

### Step E. Procedure for Preparation of 6-(8-(benzo[d]thiazol-2-ylcarbamoyl)-3,4-dihydroisoquinolin-2 (1H)-yl)-3-(2-methyl-3-(4-((R)-1,1,1-trifluoro-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)phenyl)picolinic acid [6-(8-(benzo[d]thiazol-2-ylcarbamoyl)-3,4-dihydroisoquinolin-2 (1H)-yl)-3-(2-methyl-3-(4-((R*)-1,1,1-trifluoro-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)phenyl)picolinic acid]

To a solution of tert-butyl 6-(8-(benzo[d]thiazol-2-ylcarbamoyl)-3,4-dihydroisoquinolin-2 (1H)-yl)-3-(2-methyl-3-(4-((R)-1,1,1-trifluoro-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)phenyl)picolinate (40 mg, 32.01 mol, 1 equiv.) in DCM (1 mL) was added TFA (3.65 mg, 32.01 μ mol, 2.38 μL, 1 equiv.). The mixture was stirred at 25° C. for 1 hour. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2 (1H)-yl)-3-(2-methyl-3-(4-((R)-1,1,1-trifluoro-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)phenyl)picolinic acid [6-(8-(benzo[d]thiazol-2-ylcarbamoyl)-3,4-dihydroisoquinolin-2 (1H)-yl)-3-(2-methyl-3-(4-((R*)-1,1,1-trifluoro-5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentan-2-yl)phenoxy)phenyl)picolinic acid](18.1 mg, 14.0 mol, 43.7% yield, 92.2% purity) as a white solid

MS (ESI) m/z: 597.4 [M12+H]⁺.

¹H NMR (400 MHz, DMSO-d₆) δ=12.94-12.80 (m, 1H), 12.65 (d, J=9.3 Hz, 1H), 9.00-8.96 (m, 1H), 8.35 (d, J=7.9 Hz, 1H), 8.04 (d, J=7.6 Hz, 1H), 7.90 (d, J=9.2 Hz, 1H), 7.79 (d, J=7.2 Hz, 1H), 7.66-7.61 (m, 1H), 7.55 (d, J=8.8 Hz, 1H), 7.50-7.42 (m, 4H), 7.41-7.35 (m, 4H), 7.34-7.27 (m, 3H), 7.24-7.19 (m, 1H), 7.02 (d, J=8.8 Hz, 1H), 6.92 (dd, J=8.4, 11.6 Hz, 4H), 5.10 (d, J=3.6 Hz, 1H), 5.00 (s, 2H), 4.90 (t, J=7.6 Hz, 1H), 4.49 (d, J=9.2 Hz, 1H), 4.40 (t, J=8.0 Hz, 1H), 4.27 (s, 1H), 3.94 (t, J=6.0 Hz, 2H), 3.60 (s, 2H), 3.04 (t, J=5.6 Hz, 2H), 2.45 (s, 3H), 2.22-2.07 (m, 3H), 2.04-1.94 (m, 3H), 1.91 (s, 3H), 1.83-1.73 (m, 1H), 1.36 (d, J=7.0 Hz, 3H), 0.91 (s, 9H)

### Example 41. Preparation of Compound 136a

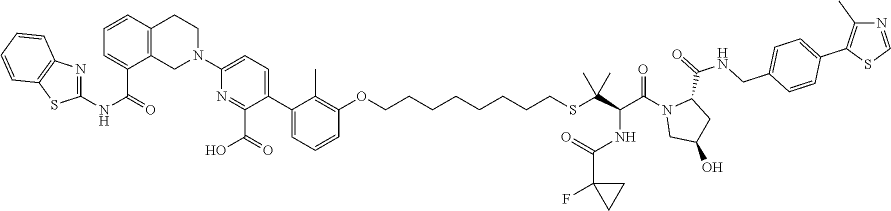 [SMILES: Cc1ncsc1-c1ccc(CNC(=O)[C@@H]2C[C@@H](O)CN2C(=O)[C@@H](NC(=O)C2(F)CC2)C(C)(C)SCCCCCCCCOc2cccc(-c3ccc(N4CCc5cccc(C(=O)Nc6nc7ccccc7s6)c5C4)nc3C(=O)O)c2C)cc1]

### 6-(8-(benzo[d]thiazol-2-ylcarbamoyl)-3,4-dihydroisoquinolin-2 (1H)-yl)-3-(3-((8-(((R)-3-(1-fluorocyclopropane-1-carboxamido)-4-((2S,4R)-4-hydroxy-2-((4-(4-methylthiazol-5-yl)benzyl)carbamoyl)pyrrolidin-1-yl)-2-methyl-4-oxobutan-2-yl)thio)octyl)oxy)-2-methylphenyl)picolinic acid

### Step A. Procedure for Preparation of tert-butyl 6-[8-[1,3-benzothiazol-2-yl(2-trimethylsilylethoxymethyl)carbamoyl]-3,4-dihydro-1H-isoquinolin-2-yl]-3-bromo-pyridine-2-carboxylate

A mixture of tert-butyl 6-[8-(1,3-benzothiazol-2-ylcarbamoyl)-3,4-dihydro-1H-isoquinolin-2-yl]-3-bromo-pyridine-2-carboxylate (2 g, 3.54 mmol, 1.0 equiv.) and TEA (1.07 g, 10.61 mmol, 1.48 mL, 3.0 equiv.) in THE (20 mL) was degassed and purged with N₂ three times. To the mixture was then added SEM-Cl (884.49 mg, 5.31 mmol, 938.95 μL, 1.5 equiv.) at 0° C. The mixture was stirred at 25° C. for 14 hours under N₂ atmosphere. The reaction mixture was concentrated under reduced pressure to give a residue. The residue was purified by flash silica gel chromatography (eluent of 16% ethyl acetate/petroleum ether) to give tert-butyl 6-[8-[1,3-benzothiazol-2-yl(2-trimethylsilylethoxymethyl)carbamoyl]-3,4-dihydro-1H-isoquinolin-2-yl]-3-bromo-pyridine-2-carboxylate (2.3 g, 3.3 mmol, 93.4% yield) as a yellow solid.

MS (ESI) m/z: 695.2 [M+H]⁺.

¹H NMR (400 MHz, DMSO-d₆) δ=8.14 (d, J=7.6 Hz, 1H), 7.95 (d, J=7.6 Hz, 1H), 7.85-7.68 (m, 3H), 7.59-7.53 (m, 1H), 7.41-7.39 (m, 2H), 6.88-6.84 (m, 1H), 5.98 (s, 2H), 5.45 (s, 1H), 5.20 (s, 2H), 4.82-4.49 (m, 1H), 3.71-3.64 (m, 2H), 3.00 (t, J=5.6 Hz, 2H), 1.45 (s, 9H), 1.32 (s, 2H), −0.19-−0.19 (m, 9H).

### Step B. Procedure for Preparation of tert-butyl 6-[8-[1,3-benzothiazol-2-yl(2-trimethylsilylethoxymethyl)carbamoyl]-3,4-dihydro-1H-isoquinolin-2-yl]-3-(4,4,5,5-tetramethyl-1,3,2-dioxaborolan-2-yl)pyridine-2-carboxylate

Tert-butyl 6-[8-[1,3-benzothiazol-2-yl(2-trimethylsilylethoxymethyl)carbamoyl]-3,4-dihydro-1H-isoquinolin-2-yl]-3-bromo-pyridine-2-carboxylate (1.15 g, 1.65 mmol, 1.0 equiv.), 4,4,5,5-tetramethyl-1,3,2-dioxaborolane (951.91 mg, 7.44 mmol, 1.08 mL, 4.5 equiv.), TEA (501.78 mg, 4.96 mmol, 690.20 μL, 3.0 equiv.), and cyclopentyl(diphenyl)phosphane;dichloropalladium;iron (120.94 mg, 165.29 mol, 0.1 equiv.) were taken up into a microwave tube in THE (10 mL) and MeCN (3.33 mL). The sealed tube was heated at 100° C. for 1 hour. The reaction mixture was filtered and concentrated under reduced pressure to give a residue. The residue was purified by flash silica gel chromatography (eluent of 16% ethyl acetate/petroleum ether) to give tert-butyl 6-[8-[1,3-benzothiazol-2-yl(2-trimethylsilylethoxymethyl)carbamoyl]-3,4-dihydro-1H-isoquinolin-2-yl]-3-(4,4,5,5-tetramethyl-1,3,2-dioxaborolan-2-yl)pyridine-2-carboxylate (1.8 g, 2.5 mmol, 76.1% yield) as a yellow solid.

MS (ESI) m/z: 743.4 [M+H]⁺.

¹H NMR (400 MHz, DMSO-d₆) δ=8.12 (d, J=6.8 Hz, 1H), 7.94 (d, J=7.6 Hz, 1H), 7.74-7.66 (m, 2H), 7.59-7.46 (m, 2H), 7.43-7.37 (m, 3H), 5.98 (s, 2H), 5.26 (s, 2H), 3.86 (t, J=6.0 Hz, 2H), 3.80-3.74 (m, 1H), 3.71-3.64 (m, 2H), 3.46-3.39 (m, 1H), 3.01-2.97 (m, 2H), 1.42 (s, 9H), 1.28 (s, 3H), 1.23 (s, 9H), −0.19 (s, 9H).

### Step C. Procedure for Preparation of tert-butyl 6-[8-[1,3-benzothiazol-2-yl(2-trimethylsilylethoxymethyl)carbamoyl]-3,4-dihydro-1H-isoquinolin-2-yl]-3-(3-hydroxy-2-methyl-phenyl)pyridine-2-carboxylate

To a solution of tert-butyl 6-[8-[1,3-benzothiazol-2-yl(2-trimethylsilylethoxymethyl)carbamoyl]-3,4-dihydro-1H-isoquinolin-2-yl]-3-(4,4,5,5-tetramethyl-1,3,2-dioxaborolan-2-yl)pyridine-2-carboxylate (900 mg, 1.21 mmol, 1.0 equiv.) and 3-bromo-2-methyl-phenol (226.61 mg, 1.21 mmol, 1 equiv.) in dioxane (10 mL) was added [2-(2-aminophenyl)phenyl]palladium(1+);bis(1-adamantyl)-butyl-phosphane;methanesulfonate (88.24 mg, 121.16 mol, 0.1 equiv.) and KF (1.5 M, 2.42 mL, 3.0 equiv.). The mixture was stirred at 80° C. for 1 hour, filtered, and concentrated under reduced pressure to give a residue. The residue was purified by flash silica gel chromatography (eluent of 18% ethyl acetate/petroleum ether) to give tert-butyl 6-[8-[1,3-benzothiazol-2-yl(2-trimethylsilylethoxymethyl)carbamoyl]-3,4-dihydro-1H-isoquinolin-2-yl]-3-(3-hydroxy-2-methyl-phenyl)pyridine-2-carboxylate (550 mg, 760.7 mol, 62.7% yield) as a yellow solid.

MS (ESI) m/z: 723.4 [M+H]⁺.

### Step D. Procedure for Preparation of tert-butyl 6-(8-(benzo[d]thiazol-2-yl((2-(trimethylsilyl)ethoxy)methyl)carbamoyl)-3,4-dihydroisoquinolin-2 (1H)-yl)-3-(3-((8-bromooctyl)oxy)-2-methylphenyl)picolinate

A mixture of tert-butyl 6-[8-[1,3-benzothiazol-2-yl(2-trimethylsilylethoxymethyl)carbamoyl]-3,4-dihydro-1H-isoquinolin-2-yl]-3-(3-hydroxy-2-methyl-phenyl)pyridine-2-carboxylate (130 mg, 179.81 mol, 1 equiv.), 8-bromooctan-1-ol (376.03 mg, 1.80 mmol, 308.23 μL, 10 equiv.), PPh₃ (235.81 mg, 899.07 mol, 5 equiv.) in THE (5 mL) was degassed and purged with N₂ three times, and then the DIAD (181.80 mg, 899.07 mol, 174.30 μL, 5 equiv.) was added dropwise into the mixture at 0° C. The mixture was stirred at 40° C. for 16 hours under N₂ atmosphere. The mixture was then concentrated to give a residue, which was purified by flash silica gel chromatography (eluent of 0˜30% ethyl acetate/petroleum ether). to give the compound tert-butyl 6-(8-(benzo[d]thiazol-2-yl((2-(trimethylsilyl)ethoxy)methyl)carbamoyl)-3,4-dihydroisoquinolin-2 (1H)-yl)-3-(3-((8-bromooctyl)oxy)-2-methylphenyl)picolinate (120 mg, 131.2 mol, 73.0% yield) as a white oil.

MS (ESI) m/z: 913.5 [M+H]⁺.

### Step E. Procedure for Preparation of tert-butyl 6-(8-(benzo[d]thiazol-2-yl((2-(trimethylsilyl)ethoxy)methyl)carbamoyl)-3,4-dihydroisoquinolin-2 (1H)-yl)-3-(3-((8-(((R)-3-(1-fluorocyclopropane-1-carboxamido)-4-((2S,4R)-4-hydroxy-2-((4-(4-methylthiazol-5-yl)benzyl)carbamoyl)pyrrolidin-1-yl)-2-methyl-4-oxobutan-2-yl)thio)octyl)oxy)-2-methylphenyl)picolinate

A mixture of tert-butyl 6-(8-(benzo[d]thiazol-2-yl((2-(trimethylsilyl)ethoxy)methyl)carbamoyl)-3,4-dihydroisoquinolin-2 (1H)-yl)-3-(3-((8-bromooctyl)oxy)-2-methylphenyl)picolinate (110 mg, 120.34 mol, 1 equiv.), (2S,4R)-1-((R)-2-(1-fluorocyclopropane-1-carboxamido)-3-mercapto-3-methylbutanoyl)-4-hydroxy-N-(4-(4-methylthiazol-5-yl)benzyl)pyrrolidine-2-carboxamide (90.08 mg, 168.48 mol, 1.4 equiv.), and DBU (109.92 mg, 722.04 mol, 108.83 μL, 6 equiv.) in THE (2 mL) was stirred at 25° C. for 12 hours. The reaction mixture was diluted with saturated NH₄Cl solution (5 mL) and concentrated into a turbid liquid. Then, the residue was extracted with DCM/MeOH(10:1), and the combined organic layers were filtered and concentrated to give tert-butyl 6-(8-(benzo[d]thiazol-2-yl((2-(trimethylsilyl)ethoxy)methyl)carbamoyl)-3,4-dihydroisoquinolin-2 (1H)-yl)-3-(3-((8-(((R)-3-(1-fluorocyclopropane-1-carboxamido)-4-((2S,4R)-4-hydroxy-2-((4-(4-methylthiazol-5-yl)benzyl)carbamoyl)pyrrolidin-1-yl)-2-methyl-4-oxobutan-2-yl)thio)octyl)oxy)-2-methylphenyl)picolinate (80 mg, crude) as a yellow oil.

MS (ESI) m/z: 1367.8 [M+H]⁺.

### Step F. Procedure for Preparation of 6-(8-(benzo[d]thiazol-2-ylcarbamoyl)-3,4-dihydroisoquinolin-2 (1H)-yl)-3-(3-((8-(((R)-3-(1-fluorocyclopropane-1-carboxamido)-4-((2S,4R)-4-hydroxy-2-((4-(4-methylthiazol-5-yl)benzyl)carbamoyl)pyrrolidin-1-yl)-2-methyl-4-oxobutan-2-yl)thio)octyl)oxy)-2-methylphenyl)picolinic acid

A mixture of tert-butyl 6-(8-(benzo[d]thiazol-2-yl((2-(trimethylsilyl)ethoxy)methyl)carbamoyl)-3,4-dihydroisoquinolin-2 (1H)-yl)-3-(3-((8-(((R)-3-(1-fluorocyclopropane-1-carboxamido)-4-((2S,4R)-4-hydroxy-2-((4-(4-methylthiazol-5-yl)benzyl)carbamoyl)pyrrolidin-1-yl)-2-methyl-4-oxobutan-2-yl)thio)octyl)oxy)-2-methylphenyl)picolinate (80 mg, 58.49 mol, 1 equiv.) in TFA (0.8 mL) and DCM (0.8 m1) was stirred at 35° C. for 16 hours. The mixture was filtered and concentrated under reduced pressure to give a residue. The residue was purified by prep-HPLC to give 6-(8-(benzo[d]thiazol-2-ylcarbamoyl)-3,4-dihydroisoquinolin-2 (1H)-yl)-3-(3-((8-(((R)-3-(1-fluorocyclopropane-1-carboxamido)-4-((2S,4R)-4-hydroxy-2-((4-(4-methylthiazol-5-yl)benzyl)carbamoyl)pyrrolidin-1-yl)-2-methyl-4-oxobutan-2-yl)thio)octyl)oxy)-2-methylphenyl)picolinic acid (20.2 mg, 14.8 mol, 25.3% yield, 86.5% purity) as a yellow solid.

MS (ESI) m/z: 1181.4 [M+H]⁺.

¹H NMR (400 MHz, DMSO-d₆) δ=12.93-12.46 (m, 2H), 8.96 (s, 1H), 8.56 (t, J=5.6 Hz, 1H), 8.03 (d, J=7.2 Hz, 1H), 7.79 (d, J=7.6 Hz, 1H), 7.64-7.59 (m, 1H), 7.47-7.34 (m, 10H), 7.08 (t, J=8.0 Hz, 1H), 6.96 (d, J=8.8 Hz, 1H), 6.85 (d, J=8.4 Hz, 1H), 6.62 (d, J=7.6 Hz, 1H), 4.98 (s, 2H), 4.77 (d, J=9.6 Hz, 1H), 4.48-4.35 (m, 3H), 4.23 (dd, J=5.6, 15.6 Hz, 1H), 3.95-3.89 (m, 4H), 3.74-3.62 (m, 2H), 3.02 (t, J=5.6 Hz, 2H), 2.44 (s, 3H), 2.11-2.04 (m, 1H), 1.95-1.90 (m, 1H), 1.88 (s, 3H), 1.73-1.67 (m, 2H), 1.43-1.22 (m, 23H)

### Example 42. Preparation of Compound 137a

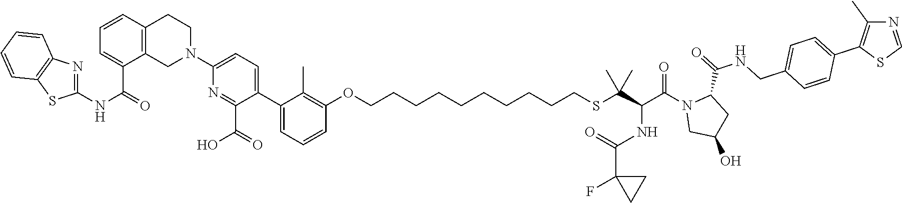 [SMILES: Cc1ncsc1-c1ccc(CNC(=O)[C@@H]2C[C@@H](O)CN2C(=O)[C@@H](NC(=O)C2(F)CC2)C(C)(C)SCCCCCCCCCCOc2cccc(-c3ccc(N4CCc5cccc(C(=O)Nc6nc7ccccc7s6)c5C4)nc3C(=O)O)c2C)cc1]

### 6-(8-(benzo[d]thiazol-2-ylcarbamoyl)-3,4-dihydroisoquinolin-2 (1H)-yl)-3-(3-((10-(((R)-3-(1-fluorocyclopropane-1-carboxamido)-4-((2S,4R)-4-hydroxy-2-((4-(4-methylthiazol-5-yl)benzyl)carbamoyl)pyrrolidin-1-yl)-2-methyl-4-oxobutan-2-yl)thio)decyl)oxy)-2-methylphenyl)picolinic acid

### Step A. Procedure for Preparation of tert-butyl 6-[8-[1,3-benzothiazol-2-yl(2-trimethylsilylethoxymethyl)carbamoyl]-3,4-dihydro-1H-isoquinolin-2-yl]-3-[3-(10-bromodecoxy)-2-methyl-phenyl]pyridine-2-carboxylate

A mixture of tert-butyl 6-[8-[1,3-benzothiazol-2-yl(2-trimethylsilylethoxymethyl)carbamoyl]-3,4-dihydro-1H-isoquinolin-2-yl]-3-(3-hydroxy-2-methyl-phenyl)pyridine-2-carboxylate (130 mg, 179.81 mol, 1 equiv.), 10-bromodecan-1-ol (426.48 mg, 1.80 mmol, 10 equiv.), DIAD (181.80 mg, 899.07 mol, 174.30 μL, 5 equiv.), and PPh₃ (235.81 mg, 899.07 mol, 5 equiv.) in THE (3 mL) was degassed and purged with N₂ three times. The mixture was then stirred at 40° C. for 12 hours under N₂ atmosphere. The reaction mixture was filtered and concentrated under reduced pressure to give a residue. The residue was purified by flash silica gel chromatography (eluent of 0˜13% ethyl acetate/petroleum ether) to give tert-butyl 6-[8-[1,3-benzothiazol-2-yl(2-trimethylsilylethoxymethyl)carbamoyl]-3,4-dihydro-1H-isoquinolin-2-yl]-3-[3-(10-bromodecoxy)-2-methyl-phenyl]pyridine-2-carboxylate (290 mg) as a yellow oil.

MS (ESI) m/z: 943.3 [M+H]⁺

### Step B. Procedure for Preparation of tert-butyl 6-(8-(benzo[d]thiazol-2-yl((2-(trimethylsilyl)ethoxy)methyl)carbamoyl)-3,4-dihydroisoquinolin-2 (1H)-yl)-3-(3-((10-(((R)-3-(1-fluorocyclopropane-1-carboxamido)-4-((2S,4R)-4-hydroxy-2-((4-(4-methylthiazol-5-yl)benzyl)carbamoyl)pyrrolidin-1-yl)-2-methyl-4-oxobutan-2-yl)thio)decyl)oxy)-2-methylphenyl)picolinate

A mixture of tert-butyl 6-[8-[1,3-benzothiazol-2-yl(2-trimethylsilylethoxymethyl)carbamoyl]-3,4-dihydro-1H-isoquinolin-2-yl]-3-[3-(10-bromodecoxy)-2-methyl-phenyl]pyridine-2-carboxylate (169 mg, 179.38 mol, 1 equiv.), (2S,4R)-1-[(2R)-2-[(1-fluorocyclopropanecarbonyl)amino]-3-methyl-3-sulfanyl-butanoyl]-4-hydroxy-N-[[4-(4-methylthiazol-5-yl)phenyl]methyl]pyrrolidine-2-carboxamide (134.27 mg, 251.13 mol, 1.4 equiv.), and DBU (163.85 mg, 1.08 mmol, 162.23 μL, 6 equiv.) in THE (5 mL) was stirred at 25° C. for 12 hours. The reaction mixture was quenched by addition sat. NH₄CL (1 mL) at 25° C., then diluted with water (3 mL), and extracted with DCM (2 mL×3). The combined organic layers were filtered and concentrated under reduced pressure to give a residue. The residue was purified by prep-TLC (SiO₂, DCM:MeOH=10:1) to give tert-butyl 6-(8-(benzo[d]thiazol-2-yl((2-(trimethylsilyl)ethoxy)methyl)carbamoyl)-3,4-dihydroisoquinolin-2 (1H)-yl)-3-(3-((10-(((R)-3-(1-fluorocyclopropane-1-carboxamido)-4-((2S,4R)-4-hydroxy-2-((4-(4-methylthiazol-5-yl)benzyl)carbamoyl)pyrrolidin-1-yl)-2-methyl-4-oxobutan-2-yl)thio)decyl)oxy)-2-methylphenyl)picolinate (30 mg, 19.3 mol, 10.7% yield, 90.1% purity) as a yellow oil

MS (ESI) m/z: 1395.5 [M+H]⁺

### Step C. Procedure for Preparation of 6-(8-(benzo[d]thiazol-2-ylcarbamoyl)-3,4-dihydroisoquinolin-2 (1H)-yl)-3-(3-((10-(((R)-3-(1-fluorocyclopropane-1-carboxamido)-4-((2S,4R)-4-hydroxy-2-((4-(4-methylthiazol-5-yl)benzyl)carbamoyl)pyrrolidin-1-yl)-2-methyl-4-oxobutan-2-yl)thio)decyl)oxy)-2-methylphenyl)picolinic acid

A mixture of tert-butyl 6-(8-(benzo[d]thiazol-2-yl((2-(trimethylsilyl)ethoxy)methyl)carbamoyl)-3,4-dihydroisoquinolin-2 (1H)-yl)-3-(3-((10-(((R)-3-(1-fluorocyclopropane-1-carboxamido)-4-((2S,4R)-4-hydroxy-2-((4-(4-methylthiazol-5-yl)benzyl)carbamoyl)pyrrolidin-1-yl)-2-methyl-4-oxobutan-2-yl)thio)decyl)oxy)-2-methylphenyl)picolinate (27 mg, 19.34 mol, 1 equiv.) and TFA (767.50 mg, 6.73 mmol, 0.5 mL, 348.00 equiv.) in DCM (1 mL) was stirred at 25° C. for 12 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2 (1H)-yl)-3-(3-((10-(((R)-3-(1-fluorocyclopropane-1-carboxamido)-4-((2S,4R)-4-hydroxy-2-((4-(4-methylthiazol-5-yl)benzyl)carbamoyl)pyrrolidin-1-yl)-2-methyl-4-oxobutan-2-yl)thio)decyl)oxy)-2-methylphenyl)picolinic acid (12.9 mg, 9.9 mol, 51.3% yield, 92.7% purity) as a yellow solid.

MS (ESI) m/z: 1209.6 [M+H]⁺

¹H NMR (400 MHz, DMSO-d₆) δ=12.95-12.34 (m, 1H), 8.97 (s, 1H), 8.56 (t, J=6.0 Hz, 1H), 8.02 (d, J=8.0 Hz, 1H), 7.78 (d, J=8.0 Hz, 1H), 7.62 (d, J=7.2 Hz, 1H), 7.49-7.32 (m, 10H), 7.11-7.03 (m, 1H), 6.98-6.91 (m, 1H), 6.85 (d, J=8.4 Hz, 1H), 6.62 (d, J=7.6 Hz, 1H), 4.98 (s, 2H), 4.77 (d, J=9.2 Hz, 1H), 4.49-4.38 (m, 2H), 4.35 (s, 1H), 4.27-4.19 (m, 1H), 3.98-3.86 (m, 4H), 3.75-3.59 (m, 2H), 3.02 (t, J=5.6 Hz, 2H), 2.44 (s, 3H), 2.11-2.04 (m, 1H), 1.96-1.84 (m, 4H), 1.75-1.66 (m, 2H), 1.42-1.31 (m, 12H), 1.27-1.15 (m, 15H).

### Example P1

### Resolution of Stereoisomeric Mixtures

Mixtures of stereoisomers were resolved using methods described in Table P1:

TABLE P1

| | | Retention | |
|---|---|---|---|
| Starting Material | Separation Method | Time | Final Product |
| 4-[4-(3-bromo-2- | Column: Chiralpak AD-3 (50 × 4.6 mm, | R_{T} = 1.051 | 129a |
| methyl- | internal diameter: 3 μm); mobile phase: | min | (Example 30) |
| phenoxy)phenyl]pentan- | Phase A (CO₂), and Phase B (MeOH with | R_{T} = 1.122 | 129b |
| 1-ol | 0.05% diethylamine); gradient elution: | min | (Example 31) |
| | MeOH (0.05% diethylamine) in CO₂ from | | |
| | 5% to 40%; flow rate: 3 mL/min; detector: | | |
| | PDA; wavelength: 220; column | | |
| | temperature: 35° C.; back pressure: 100 bar | | |
| ethyl 3-[[4-(3-bromo-2- | Column: Chiralpak AD-3 (50 × 4.6 mm, | R_{T} = 0.500 | 131a |
| methyl- | internal diameter: 3 μm); mobile phase: | min | (Example 33) |
| phenoxy)phenyl]methyl]- | Phase A (CO₂), and Phase B (MeOH with | R_{T} = 0.583 | 131b |
| 4,4,4-trifluoro- | 0.05% diethylamine); gradient elution: | min | (Example 34) |
| butanoate | MeOH (0.05% diethylamine) in CO₂ from | | |
| | 5% to 20%; flow rate: 3 mL/min; detector: | | |
| | PDA; wavelength: 220; column | | |
| | temperature: 35° C.; back pressure: 100 bar | | |
| ethyl 4-[4-(3-bromo-2- | Column: Daicel ChiralPak AY-3 (50 × | R_{T} = 0.686 | 134a |
| methyl- | 4.6 mm, internal diameter: 3 μm); mobile | min | (Example 37) |
| phenoxy)phenyl]-3- | phase: Phase A (CO₂), and Phase B (EtOH | R_{T} = 0.810 | 134b |
| methyl-butanoate | with 0.05% diethylamine); gradient | min | (Example 38) |
| | elution: MeOH (0.05% diethylamine) in | | |
| | CO₂ from 5% to 40%; flow rate: 3 mL/min; | | |
| | detector: PDA; wavelength: 220; column | | |
| | temperature: 35° C.; back pressure: 100 bar | | |
| 4-(4-(3-bromo-2- | Column: Daicel ChiralPak AS-3 (50 × | R_{T} = 0.858 | 135a |
| methylphenoxy)phenyl)- | 4.6 mm, internal diameter: 3 μm); mobile | min | (Example 39) |
| 5,5,5-trifluoropentanal | phase: Phase A (CO₂), and Phase B (EtOH | R_{T} = 1.042 | 135b |
| | with 0.05% diethylamine); gradient | min | (Example 40) |
| | elution: MeOH (0.05% diethylamine) in | | |
| | CO₂ from 5% to 40%; flow rate: 3 mL/min; | | |
| | detector: PDA; column temperature: 35° C.; | | |
| | back pressure: 100 bar | | |
| 4-(4-(3-bromo-2- | Column: Chiralcel OD-3 (50 × 4.6 mm, | R_{T} = 1.196 | 143a |
| methylphenoxy)phenyl)- | internal diameter: 3 μm); mobile phase: | min | (Example 49) |
| 2-methylbutanoic acid | Phase A (CO₂), and Phase B (MeOH with | R_{T} = 1.288 | 143b |
| | 0.05% diethylamine); gradient elution: B | min | (Example 50) |
| | in A from 10% to 30%; flow rate: | | |
| | 3 mL/min; detector: PDA; wavelength: | | |
| | 220; column temperature: 35° C.; back | | |
| | pressure: 100 bar | | |

### Example B1

### HiBiT Based Degradation Assay

Human BCL-X_{L} protein coding open reading frame fused with N-terminal HiBiT coding sequence was synthesized from Integrated DNA Technologies (IDT). Next, N-HiBiT-BCL-X_{L} sequence was cloned into pLV-UBC-PGK-Puro, a lentivirus plasmid purchased from Vectorbuilder, to generate pLV-UBC-N-HiBiT-BCL-X_{L}-PGK-puro. Lentiviral particles were generated from Lenti-X™ 293T cells (Clontech) by co-transfection of pLV-UBC-N-HiBiT-BCL-X_{L}-PGK-puro plasmids and lentiviral packaging plasmid mix (Cellecta). HT-1080 [HT1080](ATCC CCL-121) cells were infected with the lentivirus, and HT1080 cells stably integrated with the lentiviral vectors were established by incubation with 1 μg/mL puromycin (Thermofisher).

HT1080 cells expressing N-terminal HiBiT tagged BCL-X_{L} were dispensed into a 384-well plate pre-spotted with compounds at varying concentrations. Three thousand cells were seeded into each well in 40 μL of RPMI 1640 media. After 6 hours of incubation at 37° C. with 500 CO₂, 30 μL of the NANO-GLO® HiBiT Lytic Detection System working solution (Promega) was added to each well and incubated at room temperature for 30 min. After incubation, luminescence was read on a PHERAstar FSX Plate Reader (BMG). BCL-X_{L} degradation at each indicated concentration was normalized with the DMSO control. The BCL-X_{L} degradation curves were plotted using a four-parameter logistic model. For compounds displaying hook effects, the curve fitting was performed using the bottom point of the hook curves (i.e., the point of Yₘᵢₙ) as the bottom plateau to calculate EC₅₀. Yₘᵢₙ and EC₅₀ values for certain compounds are shown in Table B1.

TABLE B1

| Compound No. | Yₘᵢₙ (24 h) | EC₅₀ (24 h) | Yₘᵢₙ (6 h) | EC₅₀ (6 h) |
|---|---|---|---|---|
| 101a | | | A | + |
| 102a | | | A | ++ |
| 103a | | | A | ++ |
| 104a | | | B | + |
| 105a | A | ++ | A | ++ |
| 106a | A | ++ | A | ++ |
| 107a | | | B | ++ |
| 108a | A | ++ | A | ++ |
| 110a | | | A | +++ |
| 111a | | | B | + |
| 112a | B | ++ | B | ++ |
| 113a | A | +++ | A | ++ |
| 114a | A | ++ | B | + |
| 115a | A | +++ | A | ++ |
| 116a | A | +++ | | |
| 116b | B | ++ | | |
| 117a | B | ++ | | |
| 118a | B | + | | |
| 119a | B | + | | |
| 120a | A | ++ | | |
| 121a | A | ++ | | |
| 122a | B | + | | |
| 123a | A | +++ | | |
| 124a | A | ++ | | |
| 125a | A | + | | |
| 126a | A | ++ | | |
| 127a | A | +++ | | |
| 128a | C | ++ | | |
| 129a | A | ++ | | |
| 129b | A | +++ | | |
| 130a | A | ++ | | |
| 131a | A | + | | |
| 131b | B | + | | |
| 132a | B | + | | |
| 133a | B | ++ | | |
| 134a | A | ++ | | |
| 134b | A | +++ | | |
| 135a | A | ++ | | |
| 135b | A | ++ | | |
| 136a | A | + | | |
| 137a | B | + | | |
| 138a | A | + | | |
| 138b | C | | | |
| 139a | A | +++ | | |
| 140a | B | ++ | | |
| 141a | A | ++ | | |
| 142a | B | + | | |
| 143a | A | ++ | | |
| 143b | A | ++ | | |
| 144a | A | +++ | | |
| 144b | C | ++ | | |

Notes:
(1) “A”: Yₘᵢₙ < 50%; “B”: 50% ≤ Yₘᵢₙ < 70%; “C”: Yₘᵢₙ ≥ 70%
(2) “+++”: EC₅₀ < 100 nM; “++”: 100 nM ≤ EC₅₀ < 1000 nM; “+”: 1000 nM ≤ EC₅₀ < 10,000 nM

### Example B2

### Western Blotting Based Degradation Assay

BCL-X_{L} degradation in human cancer cell lines is measured using traditional Western blotting methods as well as with the automated “Jess” instrument from ProteinSimple. Cells are seeded at 0.5-1 million cells per well in up to 2 mL of growth media (RPMI+1000 FBS) in 6-well plates. The plates are treated with compounds at varying concentrations for up to 5 days. Cells are harvested and lysed using RIPA Buffer (#9806S, Cell Signaling Technology) with Halt Protease & Phosphatase Inhibitor Cocktail (#78441, Thermo Fisher). Protein concentrations of each sample are quantified using Pierce™ BCA Protein Assay Kit (#23225, Thermo Fisher). For the Jess instrument the standard protocol provided by the manufacturer using the Jess Separation Module SM-W004 is followed. For traditional Western blotting, samples resolved on a 4-20% Criterion TGX gel (#5671094, BioRad) and transferred to a PVDF membrane using the Trans-Blot Turbo Transfer system (#1704157, BioRad). Chemiluminescent detection of BCL-X_{L} is performed using a BCL-X_{L} antibody (#2764, Cell Signaling Technology) according to the manufacturer's instructions.

### Example B3

### MOLT-4 Proliferation Assay

MOLT-4 (ATCC CRL-1582) cells are seeded at 4,000 cells per well in 50 μL RPMI 1640 media in black 384-well plates. The plates are pre-spotted with compounds at varying concentrations. After 3 days of incubation at 37° C. with 5% CO₂, cells are assessed using CELLTITERGLO® Cell Viability Assay kit according to manufacturer's instructions (Promega). Relative cell proliferation at each concentration is normalized against the DMSO control.

### Example B4

### Cell Line Proliferation Assay

Human cancer cell lines are seeded at 1,000-4,000 cells per well in 50 μL growth media (following culture conditions recommended by cell line sources) in 384-well plates. The plates are treated with compounds at varying concentrations using a D300e Digital Dispenser (Tecan) and then incubated at 37° C. with 5% CO₂ for 3-7 days. Relative cell viability is assessed using CELLTITERGLO® Cell Viability Assay (#G9243, Promega) according to the manufacturer's instructions or Incucyte SX5 Live-Cell Analysis Instrument (Sartorius) with images recorded at 4-12 hour intervals.

### Example B5

### Apoptosis Assay

Human cancer cell lines are seeded at 1,000-4,000 cells per well in 50 μL growth media (following culture conditions recommended by cell line sources) in 384-well plates. The plates are treated with compounds at varying concentrations using a D300e Digital Dispenser (Tecan) and then incubated at 37° C. with 5% CO₂ for 3-7 days. Relative caspase- 3/7 activity is assessed using Caspase-Glo 3/7 Assay System (#G8093, Promega) according to the manufacturer's instructions or Incucyte SX5 Live-Cell Analysis Instrument (Sartorius) with Green Incucyte Caspase-3/7 Dye (#4440, Sartorius) added to the media at between 1:500 and 1:1000 dilution at the time of seeding and images recorded at 4-12 hour intervals. Relative annexin V positivity is assessed using Incucyte SX5 Live-Cell Analysis Instrument (Sartorius) with Orange Incucyte Annexin V Dye (#4759, Sartorius) added to the media at a 1:200 dilution at the time of seeding and images recorded at 4-12 hour intervals.

### Example B6

### Platelet Viability Assay

Human platelet-rich plasma (PRP, SER-PRP-SDS) is obtained from Zenbio. Sodium citrate is used as anticoagulant. To remove any contaminating blood cells, the samples are spun at 200×g at room temperature for 20 minutes in 50 mL falcon tubes, and the supernatant is transferred to a new tube. One hundred microliter PRP are added to each well of a 96-well plate spotted with compounds at varying concentrations. Plates are incubated at 37° C. with 5% CO₂ for 24 hours, and platelet viability are read with CELLTITERGLO® Cell Viability Assay kit according to manufacturer's instructions (Promega). Relative viability at each concentration is normalized against the DMSO control.

### Exemplary Embodiments

### P01 Embodiments

Embodiment 1. A compound of Formula (I) or (II):

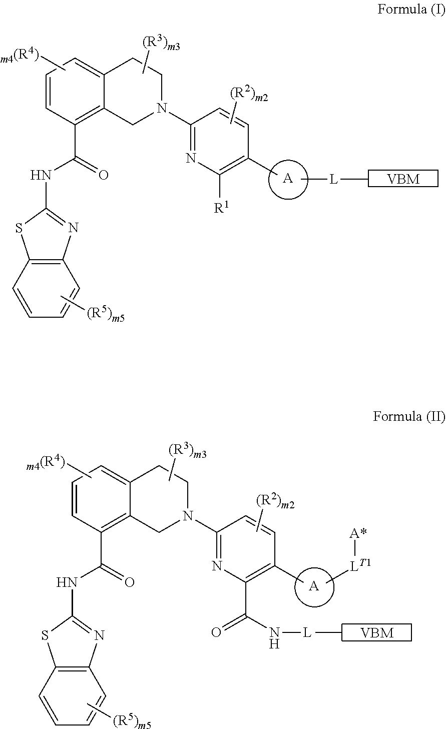

or a pharmaceutically acceptable salt thereof, wherein:
Ring A is selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) phenylene or 5-6 membered heteroarylene, each of which is optionally substituted with 1-3 substituents independently selected from the group consisting of: R^{a} and R^{b};
L^{T1} is a bond or C₁-3 alkylene optionally substituted with 1-3 substituents independently selected from the group consisting of: oxo and R^{c}, wherein one CH₂ unit of the C₁₋₃ alkylene is optionally replaced with —O— or —N(R^{d})—;
A* is selected from the group consisting of:
(a) C₃₋₁₅ cycloalkyl or 3-15 membered heterocyclyl, each of which is optionally substituted with 1-6 substituents independently selected from the group consisting of: R^{a} and R^{b};
(b) C₆₋₁₅ aryl or 5-15 membered heteroaryl, each of which is optionally substituted with 1-6 substituents independently selected from the group consisting of: R^{a} and R^{b}; and
(c) H;
R¹ is selected from the group consisting of:
(a) C(O)OH,
(b) C(O)NR^{d}R^{e}, and
(c) C(O)OC₁₋₆ alkyl, wherein the C₁₋₆ alkyl is optionally substituted with 1-3 R^{c};
each R², R³, R⁴, and R⁵ is independently selected from the group consisting of: halo, CN, C₁₋₃ alkyl, C₁₋₃ haloalkyl, C₁₋₃ alkoxy, C₁₋₃ haloalkoxy, OH, and NR^{d}R^{e};
m2 is 0, 1, or 2;
m3 and m4 are independently 0, 1, 2, or 3;
m5 is 0, 1, 2, 3, or 4;
L is -(L^{A})ₙ₁-, wherein L^{A} and n1 are defined according to (AA) or (BB):
   (AA)
n1 is an integer from 3 to 12; and
each L^{A} is independently selected from the group consisting of: L^{A1}, L^{A3}, and L^{A4} provided that 1-3 occurrences of L^{A} is L^{A4};
   (BB)
n1 is an integer from 0 to 20; and
each L^{A} is independently selected from the group consisting of: L^{A1} and L^{A3};
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any N—O, N—N, N—S(O)₀, or O—S(O)₀₋₂ bonds;
wherein each R^{L} is independently selected from the group consisting of: halo; cyano; —OH; —C₁₋₆ alkoxy; —C₁₋₆ haloalkoxy; —NR^{d}R^{c}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{c};
VBM is selected from the group consisting of: (V1), (V2), (V3), and (V4)

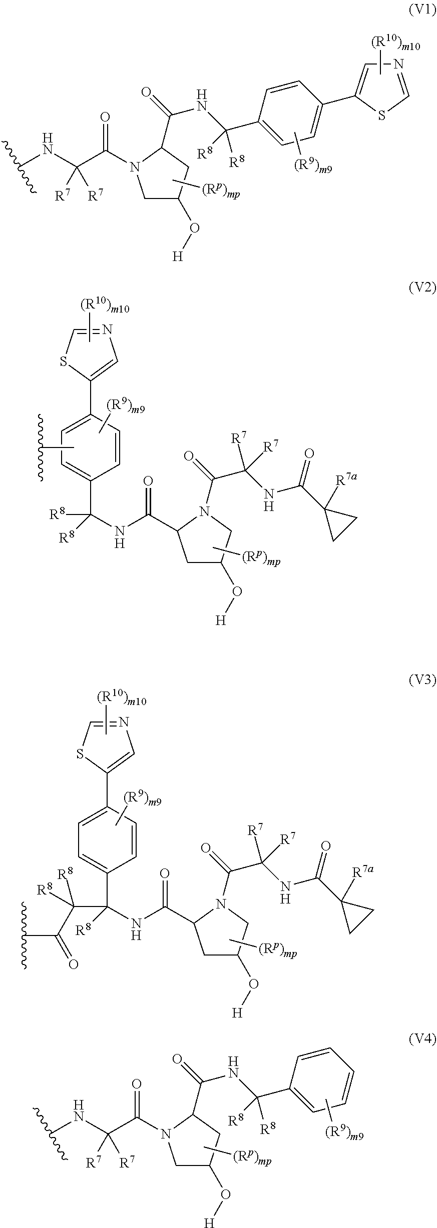

wherein:
each R⁷ and R¹ is independently selected from the group consisting of: H, C₁₋₆ alkyl, C₁₋₆ haloalkyl, and C₃₋₆ cycloalkyl;
R^{7a} is selected from the group consisting of: halo and CN;
mp is 0, 1, or 2;
each R^{p} is independently selected from the group consisting of: —F, C₁₋₆ alkyl, C₁₋₆ haloalkyl, and C₃₋₆ cycloalkyl;
m9 is 0, 1, 2, or 3;
m10 is 0, 1, or 2; and
each R⁹ and R⁰ is independently selected from the group consisting of: halo, CN, C₁₋₆ alkyl, C₁₋₆ haloalkyl, C₃₋₆ cycloalkyl, C₁₋₆ alkoxy, and C₁₋₆ haloalkoxy;
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1, 2, or 3;
each -L^{h} is independently selected from the group consisting of: —O—; —N(H)—; —N(C₁₋₃ alkyl)-; —S(O)₀₋₂—; C(═O); and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{c}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(R^{f})₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂;
provided that when VBM is (V1), (V2), or (V3), then:
(i) in Formula (I), L does not contain an adamantylene group; and
(ii) in Formula (II), A* is other than adamantyl.

Embodiment 2. The compound of Embodiment 1, wherein the compound is a compound of Formula (I), or a pharmaceutically acceptable salt thereof.

Embodiment 3. The compound of Embodiment 1, wherein the compound is a compound of Formula (II), or a pharmaceutically acceptable salt thereof.

Embodiment 4. The compound of any one of Embodiments 1-3, wherein Ring A is a phenylene optionally substituted with 1-3 R^{a}.

Embodiment 5. The compound of any one of Embodiments 1-4, wherein Ring A is

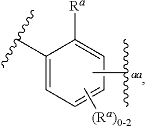

wherein aa represents the point of attachment to L or L^{T1}.

Embodiment 6. The compound of any one of Embodiments 1-5, wherein Ring A is

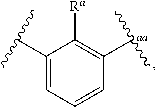

wherein aa represents the point of attachment to L or L^{T1}.

Embodiment 7. The compound of any one of Embodiments 1-5, wherein Ring A is

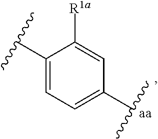

wherein aa represents the point of attachment to L or L^{T1}.

Embodiment 8. The compound of any one of Embodiments 1-3, wherein Ring A is 5-6 membered heteroarylene optionally substituted with 1-3 R^{a}.

Embodiment 9. The compound of any one of Embodiments 1-3 or 8, wherein Ring A is 5-6 membered heteroarylene optionally substituted with 1-2 R^{a}.

Embodiment 10. The compound of any one of Embodiments 1-3 or 8-9, wherein Ring A is 5-membered heteroarylene optionally substituted with 1-2 R^{a}.

Embodiment 11. The compound of any one of Embodiments 1-3 or 8-10, wherein Ring A is pyrazolylene optionally substituted with 1-2 R^{a}.

Embodiment 12. The compound of any one of Embodiments 1-3 or 8-11, wherein Ring A is selected from the group consisting of:

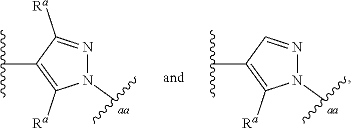

wherein aa represents the point of attachment to L or L^{T1}.

Embodiment 13. The compound of any one of Embodiments 1-3, wherein Ring A is C₃₋₁₀ cycloalkylene optionally substituted with 1-6 R^{a}.

Embodiment 14. The compound of any one of Embodiments 1-13, wherein one R^{a} present on Ring A is C₁₋₃ alkyl optionally substituted with 1-3 F.

Embodiment 15. The compound of any one of Embodiments 1-14, wherein one R^{a} present on Ring A is methyl or CF₃.

Embodiment 16. The compound of any one of Embodiments 1-2 or 4-15, wherein R¹ is C(O)OH.

Embodiment 17. The compound of any one of Embodiments 1-2 or 4-15, wherein R¹ is C(O)NHR^{e}.

Embodiment 18. The compound of any one of Embodiments 1 or 3-15, wherein L^{T1} is C₁₋₃ alkylene.

Embodiment 19. The compound of any one of Embodiments 1, 3-15, or 18, wherein L^{T1} is —CH₂—.

Embodiment 20. The compound of any one of Embodiments 1, 3-15, or 18-19, wherein A* is H.

Embodiment 21. The compound of any one of Embodiments 1, 3-15, or 18-19, wherein A* is C₃₋₁₅ cycloalkyl or 3-15 membered heterocyclyl, each of which is optionally substituted with 1-6 substituents independently selected from the group consisting of: R^{a} and R^{b}.

Embodiment 22. The compound of any one of Embodiments 1, 3-15, or 18-19, wherein A* is C₆₋₁₅ aryl or 5-15 membered heteroaryl, each of which is optionally substituted with 1-6 substituents independently selected from the group consisting of: R^{a} and R^{b}.

Embodiment 23. The compound of any one of Embodiments 1, 3-15, 18-19, or 22, wherein A* is phenyl optionally substituted with 1-3 R^{a}.

Embodiment 24. The compound of any one of Embodiments 1-23, wherein m2 is 0.

Embodiment 25. The compound of any one of Embodiments 1-24, wherein m3 is 0.

Embodiment 26. The compound of any one of Embodiments 1-25, wherein m4 is 0.

Embodiment 27. The compound of any one of Embodiments 1-26, wherein m5 is 0.

Embodiment 28. The compound of any one of Embodiments 1-23, wherein m2 is 0; m3 is 0; m4 is 0; and m5 is 0.

Embodiment 29. The compound of any one of Embodiments 1-28, wherein VBM is (V1):

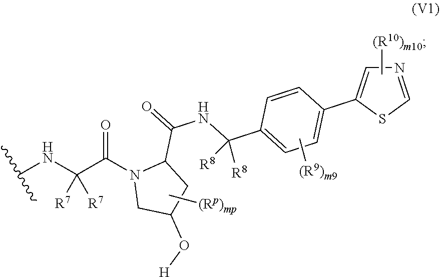

Embodiment 30. The compound of Embodiment 29, wherein VBM is (V1a):

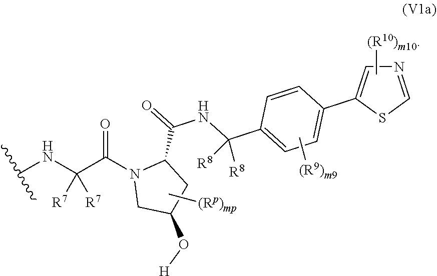

Embodiment 31. The compound of any one of Embodiments 1-28, wherein VBM is (V2):

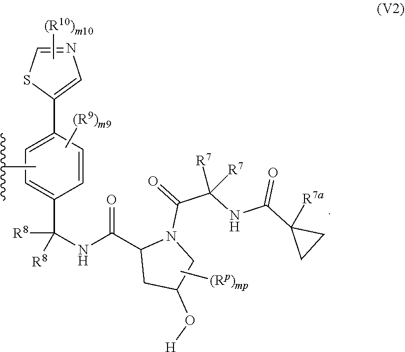

Embodiment 32. The compound of Embodiment 31, wherein VBM is (V2a):

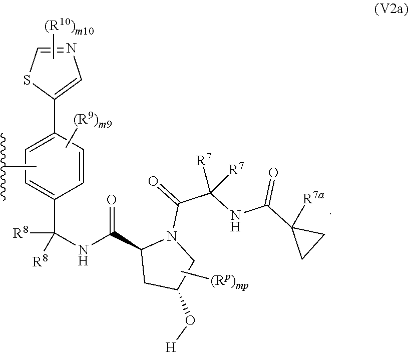

Embodiment 33. The compound of any one of Embodiments 1-28, wherein VBM is (V3):

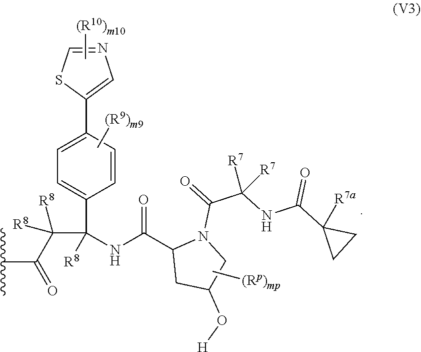

Embodiment 34. The compound of Embodiment 33, wherein VBM is (V3a):

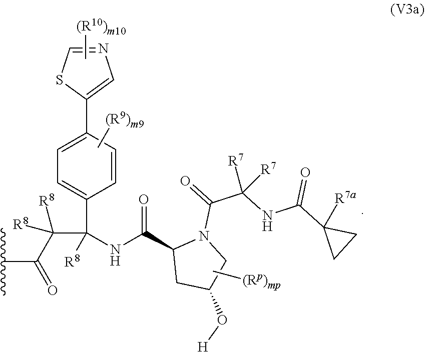

Embodiment 35. The compound of any one of Embodiments 1-28, wherein VBM is (V4):

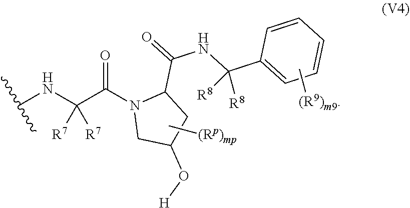

Embodiment 36. The compound of Embodiment 35, wherein VBM is (V4a):

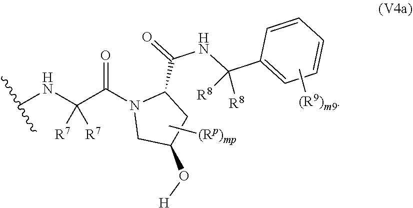

Embodiment 37. The compound of any one of Embodiments 29-36, wherein one R⁷ is H; and the other R⁷ is C₁₋₆ alkyl.

Embodiment 38. The compound of any one of Embodiments 29-37, wherein one R⁷ is H; and the other R⁷ is tert-butyl.

Embodiment 39. The compound of any one of Embodiments 29-38, wherein the carbon atom to which each R⁷ is attached has (S)-stereogenic configuration.

Embodiment 40. The compound of any one of Embodiments 29-32 or 35-39, wherein one R⁸ is H; and the other R⁸ is C₁₋₆ alkyl (e.g., methyl).

Embodiment 41. The compound of any one of Embodiments 29-39, wherein each R⁸ is H.

Embodiment 42. The compound of any one of Embodiments 29-39, wherein the carbon atom to which each R⁸ is attached has (S)-stereogenic configuration.

Embodiment 43. The compound of any one of Embodiments 29-42, wherein m9 is 0.

Embodiment 44. The compound of any one of Embodiments 29-43, wherein m10 is 1.

Embodiment 45. The compound of any one of Embodiments 29-44, wherein R¹⁰ is C₁₋₆ alkyl.

Embodiment 46. The compound of any one of Embodiments 29-45, wherein R¹⁰ is methyl.

Embodiment 47. The compound of any one of Embodiments 29-46, wherein mp is 0.

Embodiment 48. The compound of any one of Embodiments 1-30, wherein VBM is:

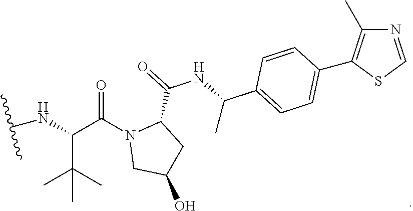 [SMILES: *N[C@H](C(=O)N1C[C@H](O)C[C@H]1C(=O)N[C@@H](C)c1ccc(-c2scnc2C)cc1)C(C)(C)C]

Embodiment 49. The compound of any one of Embodiments 1-28 or 31-32, wherein VBM is:

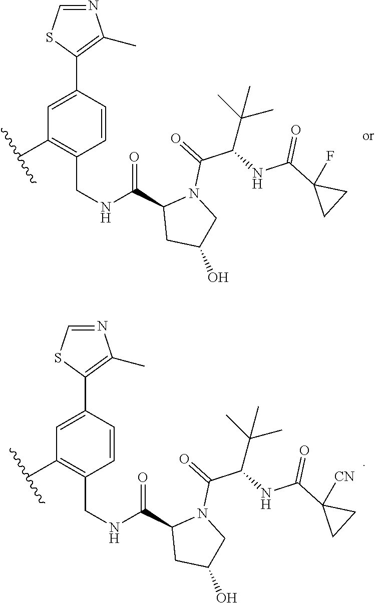 [SMILES: *c1cc(-c2scnc2C)ccc1CNC(=O)[C@@H]1C[C@@H](O)CN1C(=O)[C@@H](NC(=O)C1(F)CC1)C(C)(C)C ; *c1cc(-c2scnc2C)ccc1CNC(=O)[C@@H]1C[C@@H](O)CN1C(=O)[C@@H](NC(=O)C1(C#N)CC1)C(C)(C)C]

Embodiment 50. The compound of any one of Embodiments 1-28 or 33-34, wherein VBM is:

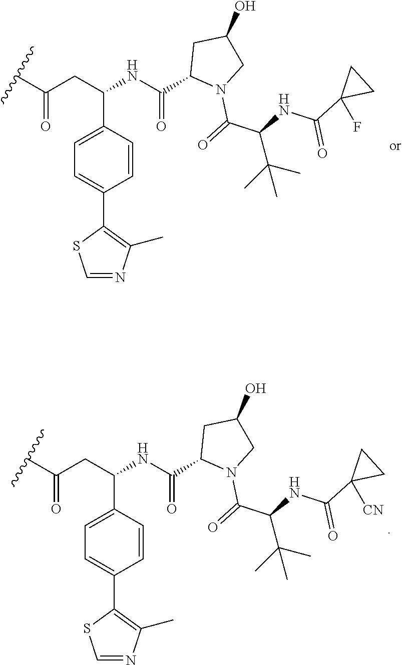 [SMILES: *C(=O)C[C@H](NC(=O)[C@@H]1C[C@@H](O)CN1C(=O)[C@@H](NC(=O)C1(F)CC1)C(C)(C)C)c1ccc(-c2scnc2C)cc1 ; *C(=O)C[C@H](NC(=O)[C@@H]1C[C@@H](O)CN1C(=O)[C@@H](NC(=O)C1(C#N)CC1)C(C)(C)C)c1ccc(-c2scnc2C)cc1]

Embodiment 51. The compound of any one of Embodiments 1-28 or 35-36, wherein VBM is:

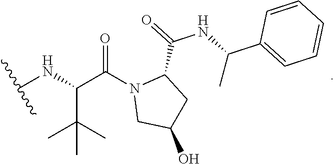 [SMILES: *N[C@H](C(=O)N1C[C@H](O)C[C@H]1C(=O)N[C@@H](C)c1ccccc1)C(C)(C)C]

Embodiment 52. The compound of any one of Embodiments 1-51, wherein L is -(L^{A})ₙ₁-; and L^{A} and n1 are defined according to (BB).

Embodiment 53. The compound of Embodiment 52, wherein n1 is an integer from 3 to 5.

Embodiment 54. The compound of Embodiment 52, wherein n1 is an integer from 5 to 9.

Embodiment 55. The compound of Embodiment 52, wherein n1 is an integer from 9 to 15.

Embodiment 56. The compound of any one of Embodiments 52-55, wherein 1-4 occurrences of L^{A} is L^{A3}.

Embodiment 57. The compound of any one of Embodiments 52-56, wherein 1-3 occurrences of L^{A} is L^{A3}.

Embodiment 58. The compound of any one of Embodiments 52-57, wherein 1-2 occurrences of L^{A} is L^{A3}.

Embodiment 59. The compound of any one of Embodiments 52-58, wherein 0-2 occurrences of L^{A1} is —CHR^{L}— or —C(R^{L})₂—; and each remaining occurrence of L^{A1} is —CH₂—.

Embodiment 60. The compound of any one of Embodiments 52-59, wherein each occurrence of L^{A1} is —CH₂—.

Embodiment 61. The compound of any one of Embodiments 52-59, wherein one occurrence of L^{A1} is —CHR^{L}— or —C(R^{L})₂—; and each remaining occurrence of L^{A1} is —CH₂—.

Embodiment 62. The compound of any one of Embodiments 1-61, wherein each R^{L} is independently selected from the group consisting of: —F and —C₁₋₃ alkyl optionally substituted with 1-3 F.

Embodiment 63. The compound of any one of Embodiments 1-52, wherein L is:
(L^{A3})₀₋₂-(L^{A1})₁₋₁₅-(L^{A3})_{0-1-bb},
wherein bb represents the point of attachment to VBM.

Embodiment 64. The compound of Embodiment 63, wherein 0-2 occurrences of L^{A1} is —CHR^{L}— or —C(R^{L})₂—; and each remaining occurrence of L^{A1} is —CH₂—.

Embodiment 65. The compound of Embodiments 63 or 64, wherein each occurrence of L^{A1} is —CH₂—.

Embodiment 66. The compound of Embodiments 63 or 64, wherein one occurrence of L^{A1} is —CHR^{L}— or —C(R^{L})₂—; and each remaining occurrence of L^{A1} is —CH₂—.

Embodiment 67. The compound of any one of Embodiments 63-66, wherein each R^{L} is independently selected from the group consisting of: —F and —C₁₋₃ alkyl optionally substituted with 1-3 F.

Embodiment 68. The compound of Embodiment 63, wherein L is a divalent group of Formula (L-1):

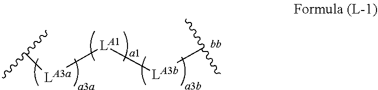

wherein:
a3a is 0 or 1;
L^{A3a} is selected from the group consisting of: —O—, —N(H)—, and —N(C₁₋₃ alkyl)-;
a1 is an integer from 1 to 15;
a3b is 0 or 1;
L^{A3b} is independently selected from the group consisting of: C(═O), —O—, N(H)—, and —N(C₁₋₃ alkyl)-; and
bb represents the point of attachment to VBM.

Embodiment 69. The compound of Embodiment 68, wherein a3b is 1.

Embodiment 70. The compound of Embodiments 68 or 69, wherein L^{A3b} is C(═O).

Embodiment 71. The compound of Embodiments 68 or 69, wherein L^{A3b} is —N(H)—.

Embodiment 72. The compound of Embodiments 68 or 69, wherein L^{A3b} is —N(C₁₋₃ alkyl)-.

Embodiment 73. The compound of Embodiments 68 or 69, wherein L^{A3b} is —O—.

Embodiment 74. The compound of Embodiment 68, wherein a3b is 0.

Embodiment 75. The compound of any one of Embodiments 68-74, wherein a3a is 1.

Embodiment 76. The compound of any one of Embodiments 68-75, wherein L^{A3}a is O.

Embodiment 77. The compound of any one of Embodiments 68-74, wherein a3a is 0.

Embodiment 78. The compound of any one of Embodiments 68-77, wherein at is 1 or 2.

Embodiment 79. The compound of any one of Embodiments 68-77, wherein at is an integer from 3 to 5.

Embodiment 80. The compound of any one of Embodiments 68-77, wherein at is an integer from 5 to 9.

Embodiment 81. The compound of any one of Embodiments 68-77, wherein at is an integer from 9 to 15.

Embodiment 82. The compound of any one of Embodiments 68-81, wherein 0-2 occurrences of L^{A1} is —CHR^{L}— or —C(R^{L})₂—; and each remaining occurrence of L^{A1} is —CH₂—.

Embodiment 83. The compound of any one of Embodiments 68-82, wherein each occurrence of L^{A1} is —CH₂—.

Embodiment 84. The compound of any one of Embodiments 68-82, wherein one occurrence of L^{A1} is —CHR^{L}— or —C(R^{L})₂—; and each remaining occurrence of L^{A1} is —CH₂—.

Embodiment 85. The compound of any one of Embodiments 82-84, wherein each R^{L} is independently selected from the group consisting of: —F and —C₁₋₃ alkyl optionally substituted with 1-3 F.

Embodiment 86. The compound of any one of Embodiments 1-51, wherein L is -(L^{A})ₙ₁-; and L^{A} and n1 are defined according to (AA).

Embodiment 87. The compound of Embodiment 86, wherein n1 is an integer from 3 to 5.

Embodiment 88. The compound of Embodiment 86, wherein n1 is an integer from 5 to 9.

Embodiment 89. The compound of Embodiments 86 or 88, wherein n1 is 6, 7, or 8.

Embodiment 90. The compound of Embodiment 86, wherein n1 is an integer from 9 to 12.

Embodiment 91. The compound of any one of Embodiments 86-90, wherein 1-2 occurrences of L^{A} is L^{A4}

Embodiment 92. The compound of any one of Embodiments 86-91, wherein one occurrence of L^{A} is L^{A4}.

Embodiment 93. The compound of any one of Embodiments 86-92, wherein each L^{A4} is independently selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

Embodiment 94. The compound of any one of Embodiments 86-93, wherein 1-4 occurrences of L^{A} is L^{A3}

Embodiment 95. The compound of any one of Embodiments 86-94, wherein 1-2 occurrences of L^{A} is L^{A3}.

Embodiment 96. The compound of any one of Embodiments 86-95, wherein 2-7 occurrences of L^{A} are L^{A1}

Embodiment 97. The compound of any one of Embodiments 86-96, wherein 2-5 occurrences of L^{A} are L^{A1}

Embodiment 98. The compound of any one of Embodiments 86-97, wherein 0-2 occurrences of L^{A1} is —CHR^{L}— or —C(R^{L})₂—; and each remaining occurrence of L^{A1} is —CH₂—.

Embodiment 99. The compound of any one of Embodiments 86-98, wherein each occurrence of L^{A1} is —CH₂—.

Embodiment 100. The compound of any one of Embodiments 86-98, wherein one occurrence of L^{A1} is —CHR^{L}— or —C(R^{L})₂—; and each remaining occurrence of L^{A1} is —CH₂—.

Embodiment 101. The compound of any one of Embodiments 98-100, wherein each R^{L} is independently selected from the group consisting of: —F and —C₁₋₃ alkyl optionally substituted with 1-3 F.

Embodiment 102. The compound of any one of Embodiments 1-51 or 86, wherein L is -(L^{A})ₙ₁-, and L^{A} and n1 are defined according to (AA), wherein:
n1 is an integer from 5 to 9;
1-2 occurrences of L^{A} is L^{A4};
2-7 occurrences of L^{A} are L^{A1}; and
1-3 occurrences of L^{A} is L^{A3}

Embodiment 103. The compound of any one of Embodiments 1-51 or 86, wherein L is selected from the group consisting of:
(i) -(L^{A3})₀₋₂-(L^{A1})₀₋₅-L^{A4}-(L^{A1})₀₋₅-L^{A3}-_{bb};
(ii) -(L^{A3})₀₋₂-(L^{A1})₀₋₅-L^{A4}-(L^{A1})_{0-5-bb}; and
provided that L contains 2-7 L^{A1}; and wherein bb represents the point of attachment to VBM.

Embodiment 104. The compound of Embodiments 102 or 103, wherein each L^{A4} is independently selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

Embodiment 105. The compound of any one of Embodiments 102-104, wherein 0-1 occurrence of L^{A1} is —CHR^{L}— or —C(R^{L})₂—; and each remaining occurrence of L^{A1} is —CH₂—.

Embodiment 106. The compound of any one of Embodiments 1-51, 86, or 103, wherein L is:
(L^{A3})₀₋₂-(L^{A1})₀₋₅-L^{A4}_A1)₀₋₅-L^{A3}-_{bb};
provided that L contains 2-7 L^{A1}; and wherein bb represents the point of attachment to VBM.

Embodiment 107. The compound of any one of Embodiments 1-51, 86, 103, or 106, wherein L is:
(L^{A3})₀₋₁-(L^{A1})₀₋₅-L^{A4}_A1)₀₋₅-L^{A3}-_{bb};
provided that L contains 2-5 L^{A1}; and wherein bb represents the point of attachment to VBM.

Embodiment 108. The compound of Embodiments 106 or 107, wherein L is a divalent group of Formula (L-2):

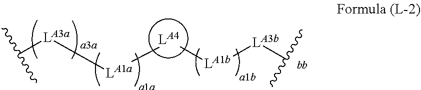

wherein:
a3a is 0 or 1;
L^{A3a} and L^{A3b} are independently selected from the group consisting of: —O—, —N(H)—, —N(C₁₋₃ alkyl)-, and C(═O);
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
bb represents the point of attachment to VBM.

Embodiment 109. The compound of Embodiment 108, wherein a3a is 1.

Embodiment 110. The compound of Embodiments 108 or 109, wherein L^{A3a} is -0-.

Embodiment 111. The compound of Embodiment 108, wherein a3a is 0.

Embodiment 112. The compound of any one of Embodiments 108-111, wherein L^{A3b} is —N(H)— or —N(C₁₋₃ alkyl)-.

Embodiment 113. The compound of any one of Embodiments 108-111, wherein L^{A3b} is —O—.

Embodiment 114. The compound of any one of Embodiments 108-111, wherein L^{A3b} is C(═O).

Embodiment 115. The compound of Embodiment 108, wherein a3a is 1; L^{A3a} is —O—; and L^{A3b} is C(═O).

Embodiment 116. The compound of Embodiment 108, wherein a3a is 1; L^{A3a} is —O—; and L^{A3b} is —O—, —N(H)—, or —N(C₁₋₃ alkyl)-.

Embodiment 117. The compound of Embodiment 108, wherein a3a is 0; and L^{A3b} is C(═O).

Embodiment 118. The compound of Embodiment 108, wherein a3a is 0; and L^{A3b} is —O—, —N(H)—, or —N(C₁₋₃ alkyl)-.

Embodiment 119. The compound of any one of Embodiments 1-51, 86, or 103, wherein L is:
-(L^{A3})₀₋₂-(L^{A1})₀₋₅-L^{A4}-(L^{A1})_{0-5-bb};
provided that L contains 2-7 L^{A1}; and wherein bb represents the point of attachment to VBM.

Embodiment 120. The compound of any one of Embodiments 1-51, 86, 103, or 119, wherein L is:
-(L^{A3})₀₋₁-(L^{A1})₀₋₅-L^{A4}-(L^{A1})_{0-5-bb};
provided that L contains 2-5 L^{A1}; and wherein bb represents the point of attachment to VBM.

Embodiment 121. The compound of Embodiment 120, wherein L is a divalent group of Formula (L-3):

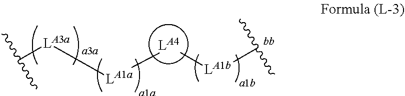

wherein:
a3a is 0 or 1;
L^{A3a} is selected from the group consisting of: —O—, —N(H)—, and —N(C₁₋₃ alkyl)-; a1a and a1b are independently integers from 0 to 5, provided that a1a+a1b is from 2 to 5;
L^{A1a} and L^{A1b} are independently selected from the group consisting of: —CH₂—, —CHR^{L}— and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
bb represents the point of attachment to VBM.

Embodiment 122. The compound of Embodiment 121, wherein a3a is 1.

Embodiment 123. The compound of Embodiments 121 or 122, wherein L^{A3a} is —O—.

Embodiment 124. The compound of Embodiment 121, wherein a3a is 0.

Embodiment 125. The compound of any one of Embodiments 108-118 or 121-124, wherein a1a+a1b is 2, 3, or 4.

Embodiment 126. The compound of any one of Embodiments 108-118 or 121-124, wherein a1a+a1b is 5.

Embodiment 127. The compound of any one of Embodiments 108-118, 121-124, or 125-126, wherein each occurrence of L^{A1a} and L^{A1b} is —CH₂—.

Embodiment 128. The compound of any one of Embodiments 108-118, 121-124, or 125-126, wherein one occurrence of L^{A1a} is —CHR^{L}— or —C(R^{L})₂—; each remaining occurrence of L^{A1a} is —CH₂—; and each occurrence of L^{A1b} is —CH₂—.

Embodiment 129. The compound of any one of Embodiments 108-118, 121-124, or 125-126, wherein one occurrence of L^{A1b} is —CHR^{L}— or —C(R^{L})₂—; each remaining occurrence of L^{A1b} is —CH₂—; and each occurrence of L^{A1a} is —CH₂—.

Embodiment 130. The compound of Embodiments 128 or 129, wherein each R^{L} is independently selected from the group consisting of: —F and —C₁₋₃ alkyl optionally substituted with 1-3 F.

Embodiment 131. The compound of any one of Embodiments 108-118 or 121-124, wherein a1b is 2, 3, or 4; one occurrence of L^{A1b} is —CHR^{L}— or —C(R^{L})₂—; each remaining occurrence of L^{A1b} is —CH₂—; and each occurrence of L^{A1a} is —CH₂—.

Embodiment 132. The compound of Embodiment 131, wherein one occurrence of L^{A1b} is CF₂, CMe₂, C(H)F, C(H)Me, or C(H)CF₃.

Embodiment 133. The compound of any one of Embodiments 106-132, wherein L^{A4} is 4-10 membered heterocyclylene optionally substituted with 1-3 R^{a}.

Embodiment 134. The compound of any one of Embodiments 106-133, wherein L^{A4} is selected from the group consisting of:

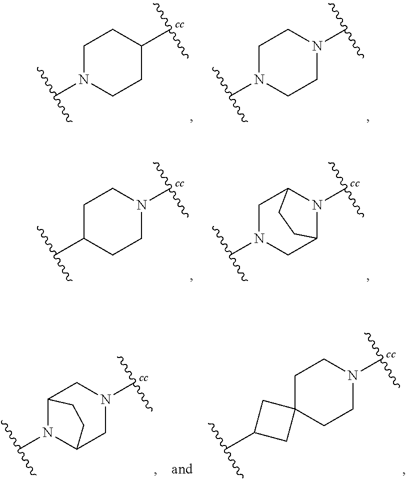 [SMILES: *C1CCN(*)CC1 ; *N1CCN(*)CC1 ; *C1CCN(*)CC1 ; *N1CC2CCC(C1)N2* ; *N1CC2CCC(C1)N2* ; *C1CC2(CCN(*)CC2)C1]

each optionally substituted with 1-3 R^{a} at one or more ring carbon atoms, wherein cc represents the point of attachment to L^{A1b}.

Embodiment 135. The compound of Embodiments 133 or 134, wherein each R^{a} present on L^{A4} is independently F or C₁₋₃ alkyl optionally substituted with 1-3 F.

Embodiment 136. The compound of any one of Embodiments 106-132, wherein L^{A4} is C₃₋₁₀ cycloalkylene optionally substituted with 1-3 R^{a}.

Embodiment 137. The compound of any one of Embodiments 106-132 or 136, wherein L^{A4} is 1,4-cyclohexylene optionally substituted with 1-3 R^{a}.

Embodiment 138. The compound of any one of Embodiments 106-132, wherein L^{A4} is phenylene or 5-6 membered heteroarylene, each of which is optionally substituted with 1-3 R^{a}.

Embodiment 139. The compound of any one of Embodiments 106-132 or 138, wherein L^{A4} is phenylene optionally substituted with 1-3 R^{a}.

Embodiment 140. The compound of any one of Embodiments 106-132 or 138-139, wherein L^{A4} is 1,2-phenylene optionally substituted with 1-3 R^{a}.

Embodiment 141. The compound of any one of Embodiments 106-132 or 138-139, wherein L^{A4} is 1,4-phenylene optionally substituted with 1-3 R^{a}.

Embodiment 142. The compound of any one of Embodiments 1-141, wherein the compound is selected from the group consisting of 101, 101a, 102, 102a, 103, 103a, 104, 104a, 105, 105a, 106, 106a, 107, 107a, 108, 108a, 109, 109a, 110, 110a, as depicted in Table C₁, or a pharmaceutically acceptable salt thereof.

Embodiment 143. A compound selected from the group consisting of 1001, 10001a, 109, and 109a as depicted in Table C2, or a pharmaceutically acceptable salt thereof.

Embodiment 144. A pharmaceutical composition comprising a compound of any one of Embodiments 1-143, or a pharmaceutically acceptable salt thereof, and a pharmaceutically acceptable excipient.

Embodiment 145. A Bcl-xL protein non-covalently bound with a compound of any one of Embodiments 1-143, or a pharmaceutically acceptable salt thereof.

Embodiment 146. A ternary complex comprising a Bcl-xL protein, a compound of any one of Embodiments 1-143, or a pharmaceutically acceptable salt thereof, and a VHL protein.

Embodiment 147. A method for treating a cancer in a subject in need thereof, the method comprising administering to the subject a therapeutically effective amount of a compound of any one of Embodiments 1-143, or a pharmaceutically acceptable salt thereof, or a pharmaceutical composition according to Embodiment 144.

Embodiment 148. A method for inducing degradation of a Bcl-xL protein in a mammalian cell, the method comprising contacting the mammalian cell with an effective amount of a compound of any one of Embodiments 1-143, or a pharmaceutically acceptable salt thereof.

Embodiment 149. The method of Embodiment 148, wherein the contacting occurs in vivo.

Embodiment 150. The method of Embodiment 148, wherein the contacting occurs in vitro.

Embodiment 151. The method of any one of Embodiments 148-150, wherein the mammalian cell is a mammalian cancer cell.

### P05 Embodiments

Embodiment 1. A compound of Formula (I):

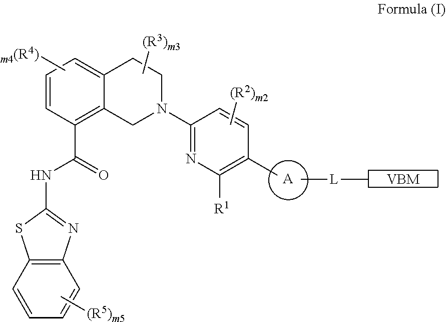

or a pharmaceutically acceptable salt thereof, wherein:
Ring A is phenylene optionally substituted with 1-3R^{a};
R¹ is selected from the group consisting of:
(a) C(O)OH,
(b) C(O)NR^{d}R^{e}, and
(c) C(O)OC₁₋₆ alkyl, wherein the C₁₋₆ alkyl is optionally substituted with 1-3 R^{c};
each R², R³, R⁴, and R⁵ is independently selected from the group consisting of: halo, CN, C₁₋₃ alkyl, C₁₋₃ haloalkyl, C₁₋₃ alkoxy, C₁₋₃ haloalkoxy, OH, and NR^{d}R^{e};
m2 is 0, 1, or 2;
m3 and m4 are independently 0, 1, 2, or 3;
m5 is 0, 1, 2, 3, or 4;
L is -(L^{A})ₙ₁-, wherein L^{A} and n1 are defined according to (AA) or (BB):
   (AA)
n1 is an integer from 3 to 12; and
each L^{A} is independently selected from the group consisting of: L^{A1}, L^{A3}, and L^{A4} provided that 1-3 occurrences of L^{A} is L^{A4};
   (BB)
n1 is an integer from 0 to 20; and
each L^{A} is independently selected from the group consisting of: L^{A1} and L^{A3};
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any O—O, N—O, N—N, N—S(O)₀, or O—S(O)₀₋₂ bonds;
wherein each R^{L} is independently selected from the group consisting of: halo; cyano; —OH; —C₁₋₆ alkoxy; —C₁₋₆ haloalkoxy; —NR^{d}R^{e}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{c};
VBM is selected from the group consisting of: (V1), (V2), (V3), (V4), (V5), (V6), (V7), (V8), and (V1P):

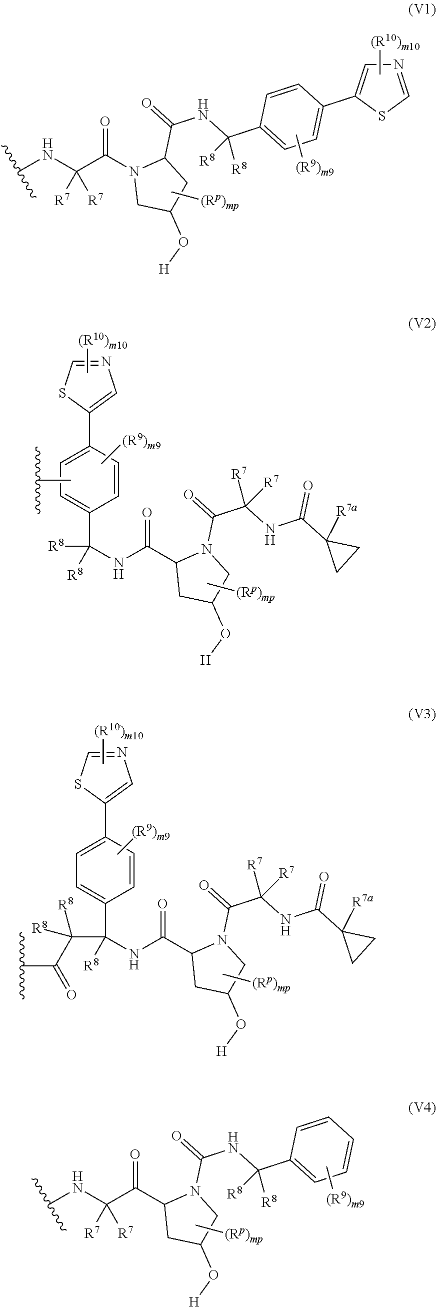

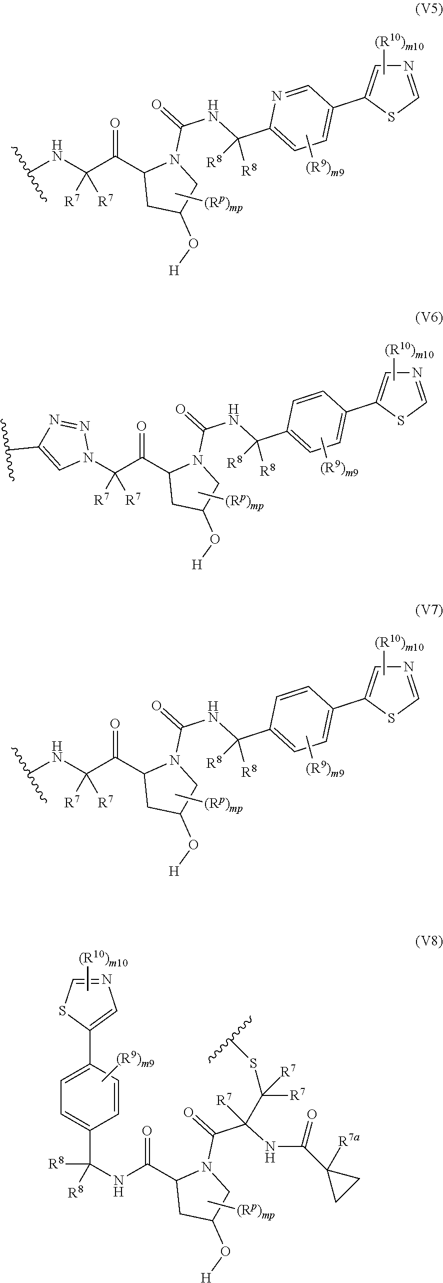

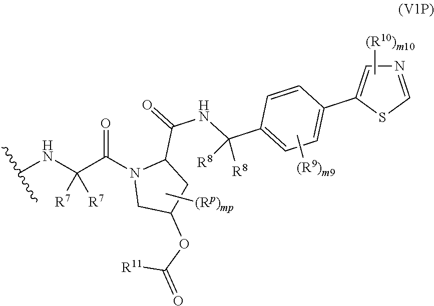

wherein:
each R⁷ and R⁸ is independently selected from the group consisting of: H, C₁₋₆ alkyl, C₁₋₆ haloalkyl, and C₃₋₆ cycloalkyl;
R^{7a} is selected from the group consisting of: halo and CN;
mp is 0, 1, or 2;
each R^{p} is independently selected from the group consisting of: —F, C₁₋₆ alkyl, C₁₋₆ haloalkyl, and C₃₋₆ cycloalkyl;
m9 is 0, 1, 2, or 3;
m10 is 0, 1, or 2; and
each R⁹ and R⁰ is independently selected from the group consisting of: halo, CN, C₁₋₆ alkyl, C₁₋₆ haloalkyl, C₃₋₆ cycloalkyl, C₁₋₆ alkoxy, and C₁₋₆ haloalkoxy;
R¹¹ is C₁₋₁₀ alkyl optionally substituted with 1-6 R^{c};
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1, 2, or 3;
each -L^{h} is independently selected from the group consisting of: —O—; —N(H)—; —N(C₁₋₃ alkyl)-; —S(O)₀₋₂—; C(═O); and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{c}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(R^{f})₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h},
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂;
provided that when VBM is (V1), (V2), or (V3), then L does not contain an adamantylene group.

Embodiment 2. The compound of Embodiment 1, wherein Ring A is

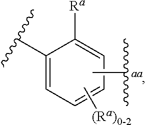

wherein aa represents the point of attachment to L.

Embodiment 3. The compound of Embodiment 1or 2, wherein Ring A is

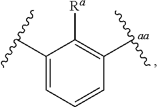

wherein aa represents the point of attachment to L.

Embodiment 4. The compound of Embodiments 1 or 2, wherein Ring A is

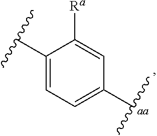

wherein aa represents the point of attachment to L.

Embodiment 5. The compound of any one of Embodiments 1-4, wherein one R^{a} present on Ring A is C₁₋₃ alkyl optionally substituted with 1-3 F.

Embodiment 6. The compound of any one of Embodiments 1-5, wherein one R^{a} present on Ring A is methyl or CF₃.

Embodiment 7. The compound of any one of Embodiments 1-6, wherein R¹ is C(O)OH.

Embodiment 8. The compound of any one of Embodiments 1-6, wherein R¹ is C(O)NHR^{e}.

Embodiment 9. The compound of any one of Embodiments 1-8, wherein m2 is 0.

Embodiment 10. The compound of any one of Embodiments 1-9, wherein m3 is 0.

Embodiment 11. The compound of any one of Embodiments 1-10, wherein m4 is 0.

Embodiment 12. The compound of any one of Embodiments 1-11, wherein m5 is 0.

Embodiment 13. The compound of any one of Embodiments 1-8, wherein m2 is 0; m3 is 0; m4 is 0; and m5 is 0.

Embodiment 14. The compound of any one of Embodiments 1-13, wherein VBM is (V1):

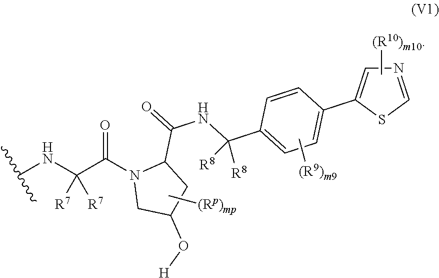

Embodiment 15. The compound of Embodiment 14, wherein VBM is (V1a):

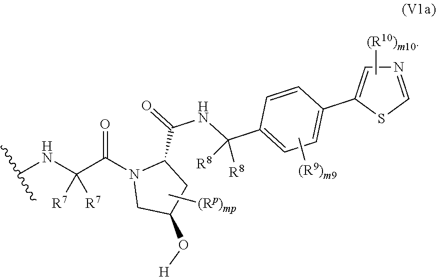

Embodiment 16. The compound of any one of Embodiments 1-13, wherein VBM is (V2):

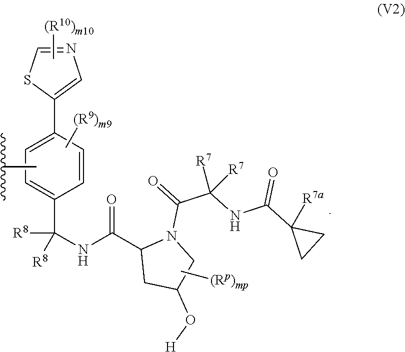

Embodiment 17. The compound of Embodiment 16, wherein VBM is (V2a):

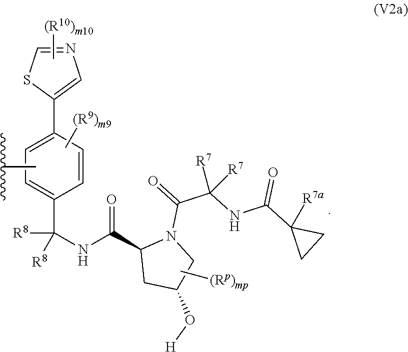

Embodiment 18. The compound of any one of Embodiments 1-13, wherein VBM is (V3):

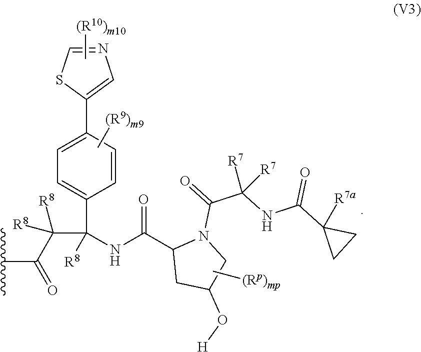

Embodiment 19. The compound of Embodiment 18, wherein VBM is (V3a):

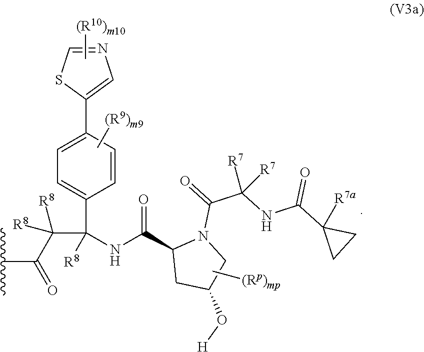

Embodiment 20. The compound of any one of Embodiments 1-13, wherein VBM is (V4):

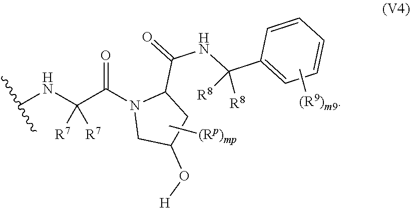

Embodiment 21. The compound of Embodiment 20, wherein VBM is (V4a):

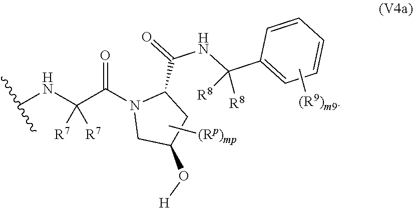

Embodiment 22. The compound of any one of Embodiments 14-21, wherein one R⁷ is H; and the other R⁷ is C₁₋₆ alkyl.

Embodiment 23. The compound of any one of Embodiments 14-22, wherein one R⁷ is H; and the other R⁷ is tert-butyl.

Embodiment 24. The compound of any one of Embodiments 14-23, wherein the carbon atom to which each R⁷ is attached has (S)-stereogenic configuration.

Embodiment 25. The compound of any one of Embodiments 14-17 or 20-24, wherein one R⁸ is H; and the other R⁸ is C₁₋₆ alkyl (e.g., methyl).

Embodiment 26. The compound of any one of Embodiments 14-24, wherein each R⁸ is H.

Embodiment 27. The compound of any one of Embodiments 14-24, wherein the carbon atom to which each R⁸ is attached has (S)-stereogenic configuration.

Embodiment 28. The compound of any one of Embodiments 14-27, wherein m9 is 0.

Embodiment 29. The compound of any one of Embodiments 14-28, wherein m10 is 1.

Embodiment 30. The compound of any one of Embodiments 14-29, wherein R¹⁰ is C₁₋₆ alkyl.

Embodiment 31. The compound of any one of Embodiments 14-30, wherein R¹⁰ is methyl.

Embodiment 32. The compound of any one of Embodiments 14-31, wherein mp is 0.

Embodiment 33. The compound of any one of Embodiments 1-15, wherein VBM is:

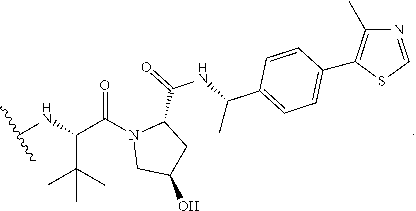 [SMILES: *N[C@H](C(=O)N1C[C@H](O)C[C@H]1C(=O)N[C@@H](C)c1ccc(-c2scnc2C)cc1)C(C)(C)C]

Embodiment 34. The compound of any one of Embodiments 1-13 or 16-17, wherein VBM is:

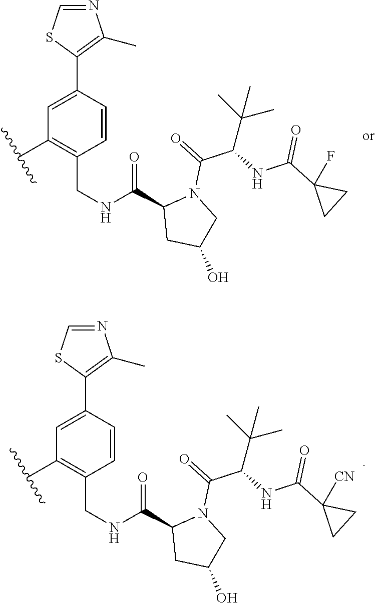 [SMILES: *c1cc(-c2scnc2C)ccc1CNC(=O)[C@@H]1C[C@@H](O)CN1C(=O)[C@@H](NC(=O)C1(F)CC1)C(C)(C)C ; *c1cc(-c2scnc2C)ccc1CNC(=O)[C@@H]1C[C@@H](O)CN1C(=O)[C@@H](NC(=O)C1(C#N)CC1)C(C)(C)C]

Embodiment 35. The compound of any one of Embodiments 1-13 or 18-19, wherein VBM is:

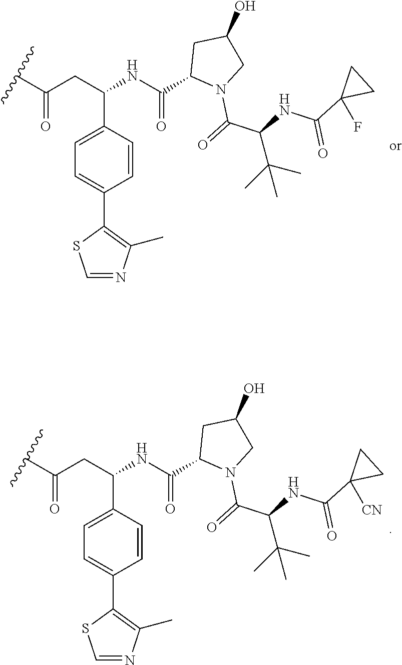 [SMILES: *C(=O)C[C@H](NC(=O)[C@@H]1C[C@@H](O)CN1C(=O)[C@@H](NC(=O)C1(F)CC1)C(C)(C)C)c1ccc(-c2scnc2C)cc1 ; *C(=O)C[C@H](NC(=O)[C@@H]1C[C@@H](O)CN1C(=O)[C@@H](NC(=O)C1(C#N)CC1)C(C)(C)C)c1ccc(-c2scnc2C)cc1]

Embodiment 36. The compound of any one of Embodiments 1-13 or 20-21, wherein VBM is:

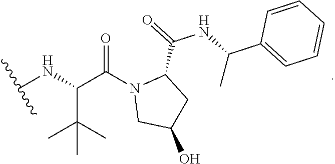 [SMILES: *N[C@H](C(=O)N1C[C@H](O)C[C@H]1C(=O)N[C@@H](C)c1ccccc1)C(C)(C)C]

Embodiment 37. The compound of any one of Embodiments 1-36, wherein L is -(L^{A})ₙ₁i-; and L^{A} and n1 are defined according to (BB).

Embodiment 38. The compound of Embodiment 37, wherein n1 is an integer from 3 to 5.

Embodiment 39. The compound of Embodiment 37, wherein n1 is an integer from 5 to 9.

Embodiment 40. The compound of Embodiment 37, wherein n1 is an integer from 9 to 15.

Embodiment 41. The compound of any one of Embodiments 37-40, wherein 1-4 occurrences of L^{A} is L^{A3}

Embodiment 42. The compound of any one of Embodiments 37-41, wherein 1-3 occurrences of L^{A} is L^{A3}

Embodiment 43. The compound of any one of Embodiments 37-42, wherein 1-2 occurrences of L^{A} is L^{A3}

Embodiment 44. The compound of any one of Embodiments 37-43, wherein 0-2 occurrences of L^{A1} is —CHR^{L}— or —C(R^{L})₂—; and each remaining occurrence of L^{A1} is —CH₂—.

Embodiment 45. The compound of any one of Embodiments 37-44, wherein each occurrence of L^{A1} is —CH₂—.

Embodiment 46. The compound of any one of Embodiments 37-44, wherein one occurrence of L^{A1} is —CHR^{L}— or —C(R^{L})₂—; and each remaining occurrence of L^{A1} is —CH₂—.

Embodiment 47. The compound of any one of Embodiments 1-46, wherein each R^{L} is independently selected from the group consisting of: —F and —C₁₋₃ alkyl optionally substituted with 1-3 F.

Embodiment 48. The compound of any one of Embodiments 1-37, wherein L is:
-(L^{A3})₀₋₂-(L^{A1})₁₋₁₅-(L^{A3})_{0-1-bb},
wherein bb represents the point of attachment to VBM.

Embodiment 49. The compound of Embodiment 48, wherein 0-2 occurrences of L^{A1} is —CHR^{L}— or —C(R^{L})₂—; and each remaining occurrence of L^{A1} is —CH₂—.

Embodiment 50. The compound of Embodiments 48 or 49, wherein each occurrence of L^{A1} is —CH₂—.

Embodiment 51. The compound of Embodiments 48 or 49, wherein one occurrence of L^{A1} is —CHR^{L}— or —C(R^{L})₂—; and each remaining occurrence of L^{A1} is —CH₂—.

Embodiment 52. The compound of any one of Embodiments 48-51, wherein each R^{L} is independently selected from the group consisting of: —F and —C₁₋₃ alkyl optionally substituted with 1-3 F.

Embodiment 53. The compound of Embodiment 48, wherein L is a divalent group of Formula (L-1):

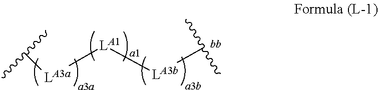

wherein:
a3a is 0 or 1;
L^{A3a} is selected from the group consisting of: —O—, —N(H)—, and —N(C₁₋₃ alkyl)-;
a1 is an integer from 1 to 15;
a3b is 0 or 1;
L^{A3b} is independently selected from the group consisting of: C(═O), —O—, N(H)—, and —N(C₁₋₃ alkyl)-; and
bb represents the point of attachment to VBM.

Embodiment 54. The compound of Embodiment 53, wherein a3b is 1.

Embodiment 55. The compound of Embodiments 53 or 54, wherein L^{A3b} is C(═O).

Embodiment 56. The compound of Embodiments 53 or 54, wherein L^{A3b} is —N(H)—.

Embodiment 57. The compound of Embodiments 53 or 54, wherein L^{A3b} is —N(C₁₋₃ alkyl)-.

Embodiment 58. The compound of Embodiments 53 or 54, wherein L^{A3b} is —O—.

Embodiment 59. The compound of Embodiment 53, wherein a3b is 0.

Embodiment 60. The compound of any one of Embodiments 53-59, wherein a3a is 1.

Embodiment 61. The compound of any one of Embodiments 53-60, wherein L^{A3}a is O.

Embodiment 62. The compound of any one of Embodiments 53-61, wherein a3a is 0.

Embodiment 63. The compound of any one of Embodiments 53-62, wherein at is 1 or 2.

Embodiment 64. The compound of any one of Embodiments 53-62, wherein at is an integer from 3 to 5.

Embodiment 65. The compound of any one of Embodiments 53-62, wherein at is an integer from 5 to 9.

Embodiment 66. The compound of any one of Embodiments 53-62, wherein at is an integer from 9 to 15.

Embodiment 67. The compound of any one of Embodiments 53-66, wherein 0-2 occurrences of L^{A1} is —CHR^{L}— or —C(R^{L})₂—; and each remaining occurrence of L^{A1} is —CH₂—.

Embodiment 68. The compound of any one of Embodiments 53-67, wherein each occurrence of L^{A1} is —CH₂—.

Embodiment 69. The compound of any one of Embodiments 53-67, wherein one occurrence of L^{A1} is —CHR^{L}— or —C(R^{L})₂—; and each remaining occurrence of L^{A1} is —CH₂—.

Embodiment 70. The compound of any one of Embodiments 67-69, wherein each R^{L} is independently selected from the group consisting of: —F and —C₁₋₃ alkyl optionally substituted with 1-3 F.

Embodiment 71. The compound of any one of Embodiments 1-36, wherein L is -(L^{A})ₙ₁-; and L^{A} and n1 are defined according to (AA).

Embodiment 72. The compound of Embodiment 71, wherein n1 is an integer from 3 to 5.

Embodiment 73. The compound of Embodiment 71, wherein n1 is an integer from 5 to 9.

Embodiment 74. The compound of Embodiments 71 or 73, wherein n1 is 6, 7, or 8.

Embodiment 75. The compound of Embodiment 71, wherein n1 is an integer from 9 to 12.

Embodiment 76. The compound of any one of Embodiments 71-75, wherein 1-2 occurrences of L^{A} is L^{A4}

Embodiment 77. The compound of any one of Embodiments 71-76, wherein one occurrence of L^{A} is L^{A4}

Embodiment 78. The compound of any one of Embodiments 71-77, wherein each L^{A4} is independently selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

Embodiment 79. The compound of any one of Embodiments 71-78, wherein 1-4 occurrences of L^{A} is L^{A3}

Embodiment 80. The compound of any one of Embodiments 71-79, wherein 1-2 occurrences of L^{A} is L^{A3}

Embodiment 81. The compound of any one of Embodiments 71-80, wherein 2-7 occurrences of L^{A} are L^{A1}

Embodiment 82. The compound of any one of Embodiments 71-81, wherein 2-5 occurrences of L^{A} are L^{A1}.

Embodiment 83. The compound of any one of Embodiments 71-82, wherein 0-2 occurrences of L^{A1} is —CHR^{L}— or —C(R^{L})₂—; and each remaining occurrence of L^{A1} is —CH₂—.

Embodiment 84. The compound of any one of Embodiments 71-83, wherein each occurrence of L^{A1} is —CH₂—.

Embodiment 85. The compound of any one of Embodiments 71-83, wherein one occurrence of L^{A1} is —CHR^{L}— or —C(R^{L})₂—; and each remaining occurrence of L^{A1} is —CH₂—.

Embodiment 86. The compound of any one of Embodiments 83-85, wherein each R^{L} is independently selected from the group consisting of: —F and —C₁₋₃ alkyl optionally substituted with 1-3 F.

Embodiment 87. The compound of any one of Embodiments 1-36 or 71, wherein L is -(L^{A})ₙ₁-, and L^{A} and n1 are defined according to (AA), wherein:
n1 is an integer from 5 to 9;
1-2 occurrences of L^{A} is L^{A4};
2-7 occurrences of L^{A} are L^{A1}; and
1-3 occurrences of L^{A} is L^{A3}

Embodiment 88. The compound of any one of Embodiments 1-36 or 71, wherein L is selected from the group consisting of:
(i) -(L^{A3})₀₋₂-(L^{A1})₀₋₅-L^{A4}-(L^{A1})₀₋₅-L^{A3}-_{bb};
(ii) -(L^{A3})₀₋₂-(L^{A1})₀₋₅-L^{A4}-(L^{A1})_{0-5-bb}; and
provided that L contains 2-7 L^{A1}; and wherein bb represents the point of attachment to VBM.

Embodiment 89. The compound of Embodiments 87 or 88, wherein each L^{A4} is independently selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

Embodiment 90. The compound of any one of Embodiments 87-89, wherein 0-1 occurrence of L^{A1} is —CHR^{L}— or —C(R^{L})₂—; and each remaining occurrence of L^{A1} is —CH₂—.

Embodiment 91. The compound of any one of Embodiments 1-36 or 71, wherein L is:
-(L^{A3})₀₋₂-(L^{A1})₀₋₅-L^{A4}-(L^{A1})₀₋₅-L^{A3}-_{bb};
provided that L contains 2-7 L^{A1}; and wherein bb represents the point of attachment to VBM.

Embodiment 92. The compound of any one of Embodiments 1-36, 71, or 91, wherein L is:
-(L^{A3})₀₋₁-(L^{A1})₀₋₅-L^{A4}-(L^{A1})₀₋₅-L^{A3}-_{bb};
provided that L contains 2-5 L^{A1}; and wherein bb represents the point of attachment to VBM.

Embodiment 93. The compound of Embodiments 91 or 92, wherein L is a divalent group of Formula (L-2):

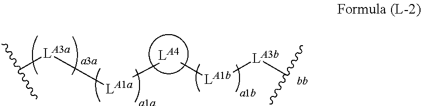

wherein:
a3a is 0 or 1;
L^{A3a} and L^{A3b} are independently selected from the group consisting of: —O—, —N(H)—, —N(C₁₋₃ alkyl)-, and C(═O);
a1a and a1b are independently integers from 0 to 5, provided that a1a+a1b is from 2 to 5;
L^{A1a} and L^{A1b} are independently selected from the group consisting of: —CH₂—, —CHR^{L}— and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
bb represents the point of attachment to VBM.

Embodiment 94. The compound of Embodiment 93, wherein a3a is 1.

Embodiment 95. The compound of Embodiments 93 or 94, wherein L^{A3a} is —O—.

Embodiment 96. The compound of Embodiment 93, wherein a3a is 0.

Embodiment 97. The compound of any one of Embodiments 93-96, wherein L^{A3b} is —N(H)— or —N(C₁₋₃ alkyl)-.

Embodiment 98. The compound of any one of Embodiments 93-96, wherein L^{A3b} is —O—.

Embodiment 99. The compound of any one of Embodiments 93-96, wherein L^{A3b} is C(═O).

Embodiment 100. The compound of Embodiment 93, wherein a3a is 1; L^{A3a} is —O—; and L^{A3b} is C(═O).

Embodiment 101. The compound of Embodiment 93, wherein a3a is 1; L^{A3a} is —O—; and L^{A3b} is —O—, —N(H)—, or —N(C₁₋₃ alkyl)-.

Embodiment 102. The compound of Embodiment 93, wherein a3a is 0; and L^{A3b} is C(═O).

Embodiment 103. The compound of Embodiment 93, wherein a3a is 0; and L^{A3b} is —O—, —N(H)—, or —N(C₁₋₃ alkyl)-.

Embodiment 104. The compound of any one of Embodiments 1-36 or 71, wherein L is:
-(L^{A3})₀₋₂-(L^{A1})₀₋₅-L^{A4}_A1)_{0-5-bb};
provided that L contains 2-7 L^{A1}; and wherein bb represents the point of attachment to VBM.

Embodiment 105. The compound of any one of Embodiments 1-36, 71, or 104, wherein L is:
-(L^{A3})₀₋₁-(L^{A1})₀₋₅-L^{A4}-(L^{A1})_{0-5-bb};
provided that L contains 2-5 L^{A1}; and wherein bb represents the point of attachment to VBM.

Embodiment 106. The compound of Embodiments 104 or 105, wherein L is a divalent group of Formula (L-3):

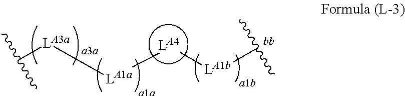

wherein:
a3a is 0 or 1;
L^{A3a} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}— and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
bb represents the point of attachment to VBM.

Embodiment 107. The compound of Embodiment 106, wherein a3a is 1.

Embodiment 108. The compound of Embodiments 106 or 107, wherein L^{A3a} is —O—.

Embodiment 109. The compound of Embodiment 106, wherein a3a is 0.

Embodiment 110. The compound of any one of Embodiments 93-103 or 106-109, wherein a1a+a1b is 2, 3, or 4.

Embodiment 111. The compound of any one of Embodiments 93-103 or 106-109, wherein a1a+a1b is 5.

Embodiment 112. The compound of any one of Embodiments 93-103, 106-109, or 110-111, wherein each occurrence of L^{A1}a and L^{A1b} is —CH₂—.

Embodiment 113. The compound of any one of Embodiments 93-103, 106-109, or 110-111, wherein one occurrence of L^{A1a} is —CHR^{L}— or —C(R^{L})₂—; each remaining occurrence of L^{A1a} is —CH₂—; and each occurrence of L^{A1b} is —CH₂—.

Embodiment 114. The compound of any one of Embodiments 93-103, 106-109, or 110-111, wherein one occurrence of L^{A1b} is —CHR^{L}— or —C(R^{L})₂—; each remaining occurrence of L^{Ab} is —CH₂—; and each occurrence of L^{A1a} is —CH₂—.

Embodiment 115. The compound of Embodiments 113 or 114, wherein each R^{L} is independently selected from the group consisting of: —F and —C₁₋₃ alkyl optionally substituted with 1-3 F.

Embodiment 116. The compound of any one of Embodiments 93-103 or 106-109, wherein a1b is 2, 3, or 4; one occurrence of L^{A1b} is —CHR^{L}— or —C(R^{L})₂—; each remaining occurrence of L^{A1b} is —CH₂—; and each occurrence of L^{A1a} is —CH₂—.

Embodiment 117. The compound of Embodiment 116, wherein one occurrence of L^{A1b} is CF₂, CMe₂, C(H)F, C(H)Me, or C(H)CF₃.

Embodiment 118. The compound of any one of Embodiments 91-117, wherein L^{A4} is 4-10 membered heterocyclylene optionally substituted with 1-3 R^{a}.

Embodiment 119. The compound of any one of Embodiments 91-118, wherein L^{A4} is selected from the group consisting of:

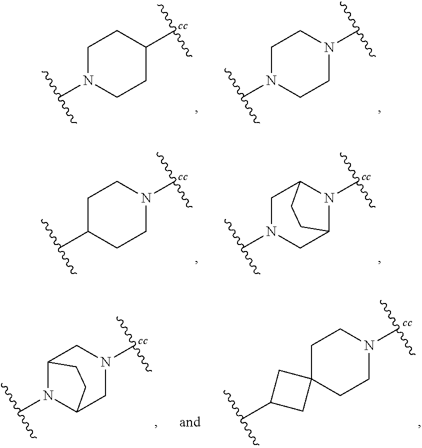 [SMILES: *C1CCN(*)CC1 ; *N1CCN(*)CC1 ; *C1CCN(*)CC1 ; *N1CC2CCC(C1)N2* ; *N1CC2CCC(C1)N2* ; *C1CC2(CCN(*)CC2)C1]

each optionally substituted with 1-3 R^{a} at one or more ring carbon atoms, wherein cc represents the point of attachment to L^{A1}b

Embodiment 120. The compound of Embodiments 118 or 119, wherein each R^{a} present on L^{A4} is independently F or C₁₋₃ alkyl optionally substituted with 1-3 F.

Embodiment 121. The compound of any one of Embodiments 91-117, wherein L^{A4} is C₃₋₁₀ cycloalkylene optionally substituted with 1-3 R^{a}.

Embodiment 122. The compound of any one of Embodiments 91-117 or 121, wherein L^{A4} is 1,4-cyclohexylene optionally substituted with 1-3 R^{a}.

Embodiment 123. The compound of any one of Embodiments 91-117, wherein L^{A4} is phenylene or 5-6 membered heteroarylene, each of which is optionally substituted with 1-3 R^{a}.

Embodiment 124. The compound of any one of Embodiments 91-117 or 123, wherein L^{A4} is phenylene optionally substituted with 1-3 R^{a}.

Embodiment 125. The compound of any one of Embodiments 91-117 or 123-124, wherein L^{A4} is 1,2-phenylene optionally substituted with 1-3 R^{a}

Embodiment 126. The compound of any one of Embodiments 91-117 or 123-124, wherein L^{A4} is 1,4-phenylene optionally substituted with 1-3 R^{a}

Embodiment 127. A compound selected from the group consisting of 101, 101a, 102, 102a, 103,103a, 104,104a, 105,105a, 106,106a, 107,107a, 108,108a, 110, 110a, 111, 111a, 112,112a, 113,113a, 114,114a, 115,115a, 116,116a, 116b, 117,117a, 118,118a, 119,119a, 120, 120a, 121, 121a, 122, 122a, 123, 123a, 124, 124a, 125, 125a, 126, 126a, 127, 127a, 128, 128a, 129, 129a, 129b, 130, 130a, 131, 131a, 131b, 132, 132a, 133, 133a, 134, 134a, 134b, 135, 135a, 135b, 136, 136a, 137, 137a, 138, 138a, 139, 139a, 140, 140a, 141, 141a, 142, 142a, 143, 143a, 143b, 144, and 144a as depicted in Table C1, or a pharmaceutically acceptable salt thereof, provided that the compound does not exhibit Yₘᵢₙ (6 h)≥70% and/or Yₘᵢₙ (24 h)≥70% under conditions described in Example B1.

Embodiment 128. A pharmaceutical composition comprising a compound of any one of Embodiments 1-127, or a pharmaceutically acceptable salt thereof, and a pharmaceutically acceptable excipient.

Embodiment 129. A BCL-X_{L} protein non-covalently bound with a compound of any one of Embodiments 1-127, or a pharmaceutically acceptable salt thereof.

Embodiment 130. A ternary complex comprising a BCL-X_{L} protein, a compound of any one of Embodiments 1-127, or a pharmaceutically acceptable salt thereof, and a VHL protein.

Embodiment 131. A method for treating a cancer in a subject in need thereof, the method comprising administering to the subject a therapeutically effective amount of a compound of any one of Embodiments 1-127, or a pharmaceutically acceptable salt thereof, or a pharmaceutical composition according to Embodiment 128.

Embodiment 132. The method of Embodiment 131, further comprising administering an additional therapy or therapeutic agent.

Embodiment 133. The method of Embodiment 132, wherein the additional therapy or therapeutic agent is an ALK inhibitor, a BCL-2 inhibitor, a BCR-Abl inhibitor, a BRaf inhibitor, a CDK2 inhibitor, CDK 4/6 inhibitor, a CDK7 inhibitor, a CDK9 inhibitor, an EGFR inhibitor, an anti-EGFR antibody, an ERK inhibitor, a FGFR1 inhibitor, a FGFR2 inhibitor, a FGFR3 inhibitor, a FGFR4 inhibitor, a HER2 inhibitor, a JAK2 inhibitor, a KRas inhibitor, a MEK inhibitor, a MET inhibitor, a PARP inhibitor, an LSD1 inhibitor, a BET inhibitor, a telomerase inhibitor, a TORC1/2 inhibitor, chemotherapy, radiotherapy, or a combination thereof.

Embodiment 134. The method of any one of Embodiments 131-133, wherein the cancer is breast cancer, colorectal cancer, bile duct cancer, colorectal cancer, gastrointestinal stromal tumor, pancreatic cancer, bladder cancer, kidney cancer, cervical cancer, ovarian cancer, uterine cancer, head and neck cancer, hematological cancer, lung cancer, skin cancer, or a combination thereof.

Embodiment 135. The method of Embodiment 134, wherein the hematological cancer is acute lymphocytic leukemia (ALL), acute myeloid leukemia (AML), chronic lymphocytic leukemia (CLL), follicular lymphoma (FL), small lymphocytic lymphoma (SLL), essential thrombocythemia, polycythemia vera, myelofibrosis, or a combination thereof.

Embodiment 136. A method for inducing degradation of a BCL-X_{L} protein in a mammalian cell, the method comprising contacting the mammalian cell with an effective amount of a compound of any one of Embodiments 1-127, or a pharmaceutically acceptable salt thereof.

Embodiment 137. The method of Embodiment 136, wherein the contacting occurs in vivo.

Embodiment 138. The method of Embodiment 136, wherein the contacting occurs in vitro.

Embodiment 139. The method of any one of Embodiments 136-138, wherein the mammalian cell is a mammalian cancer cell.

What is claimed is:

1. A compound of Formula (I): 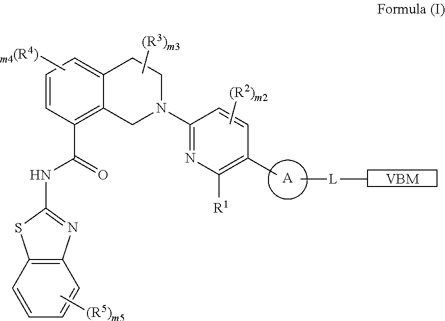
or a pharmaceutically acceptable salt thereof, wherein:
Ring A is phenylene optionally substituted with 1-3R^{a};
R¹ is selected from the group consisting of:
(a) C(O)OH,
(b) C(O)NR^{d}R^{c}, and
(c) C(O)OC₁₋₆ alkyl, wherein the C₁₋₆ alkyl is optionally substituted with 1-3 R^{c};
each R², R³, R⁴, and R⁵ is independently selected from the group consisting of: halo, CN, C₁₋₃ alkyl, C₁₋₃ haloalkyl, C₁₋₃ alkoxy, C₁₋₃ haloalkoxy, OH, and NR^{d}R^{e};
m2 is 0, 1, or 2;
m3 and m4 are independently 0, 1, 2, or 3;
m5 is 0, 1, 2, 3, or 4;
L is -(L^{A})ₙ₁-, wherein L^{A} and n1 are defined according to (AA) or (BB):
(AA)
n1 is an integer from 3 to 12; and
each L^{A} is independently selected from the group consisting of: L^{A1}, L^{A3}, and L^{A4}, provided that 1-3 occurrences of L^{A} is L^{A4};
(BB)
n1 is an integer from 0 to 20; and
each L^{A} is independently selected from the group consisting of: L^{A1} and L^{A3};
each L^{A1} is independently selected from the group consisting of: —CH₂—, —CHR^{L}—, and —C(R^{L})²—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any O—O, N—O, N—N, N—S(O)₀, or O—S(O)₀₋₂ bonds;
wherein each R^{L} is independently selected from the group consisting of: halo; cyano; —OH; —C₁₋₆ alkoxy; —C₁₋₆ haloalkoxy; —NR^{d}R^{e}; C(═O)N(R′)₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{e};
VBM is selected from the group consisting of: (V1), (V2), (V3), (V4), (V5), (V6), (V7), (V8), and (V1P): 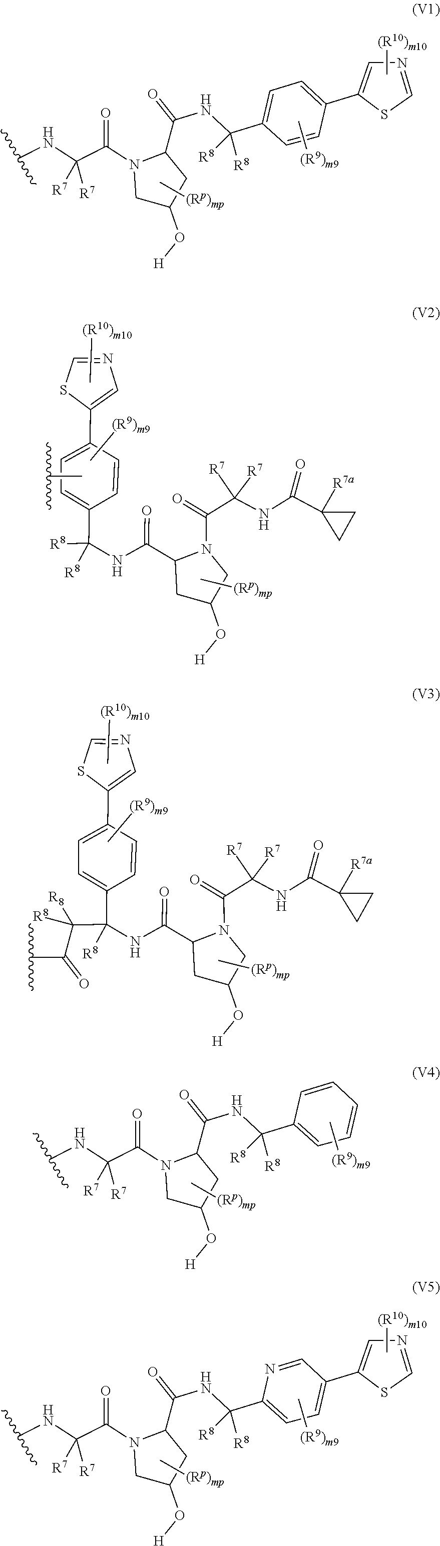 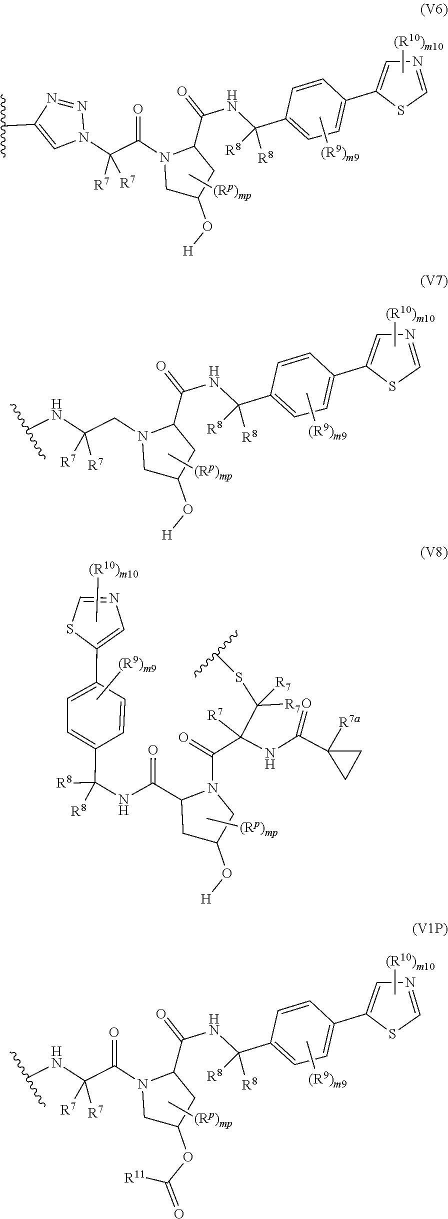
wherein:
each R⁷ and R⁸ is independently selected from the group consisting of: H, C₁₋₆ alkyl, C₁₋₆ haloalkyl, and C₃₋₆ cycloalkyl;
R^{7a} is selected from the group consisting of: halo and CN;
mp is 0, 1, or 2;
each R^{p} is independently selected from the group consisting of: —F, C₁₋₆ alkyl, C₁₋₆ haloalkyl, and C₃₋₆ cycloalkyl;
m9 is 0, 1, 2, or 3;
m10 is 0, 1, or 2; and
each R⁹ and R⁰ is independently selected from the group consisting of: halo, CN, C₁₋₆ alkyl, C₁₋₆ haloalkyl, C₃₋₆ cycloalkyl, C₁₋₆ alkoxy, and C₁₋₆ haloalkoxy;
R¹¹ is C₁₋₁₀ alkyl optionally substituted with 1-6 R^{c};
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c},
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(R^{f})₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h},
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂;
provided that when VBM is (V1), (V2), or (V3), then L does not contain an adamantylene group.

2. The compound of claim 1, wherein Ring A is 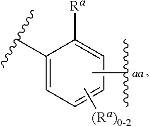
wherein aa represents the point of attachment to L; or
Ring A is 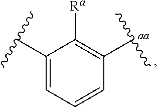
wherein aa represents the point of attachment to L; or
Ring A is 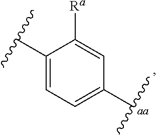
wherein aa represents the point of attachment to L.

3. The compound of claim 1 or 2, wherein one R^{a} present on Ring A is C₁₋₃ alkyl optionally substituted with 1-3 F; or
one R^{a} present on Ring A is methyl or CF₃.

4. The compound of any one of claims 1-3, wherein R¹ is C(O)OH.

5. The compound of any one of claims 1-3, wherein R¹ is C(O)NHR^{e}.

6. The compound of any one of claims 1-5, wherein m2 is 0.

7. The compound of any one of claims 1-6, wherein m3 is 0.

8. The compound of any one of claims 1-7, wherein m4 is 0.

9. The compound of any one of claims 1-8, wherein m5 is 0.

10. The compound of any one of claims 1-5, wherein m2 is 0; m3 is 0; m4 is 0; and
m5 is 0.

11. The compound of any one of claims 1-10, wherein VBM is (V1): 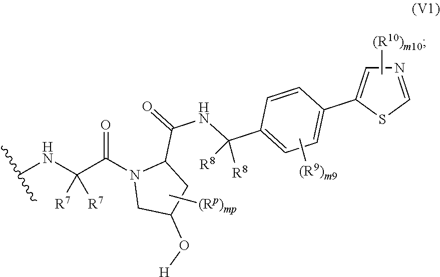
or
VBM is (V1a): 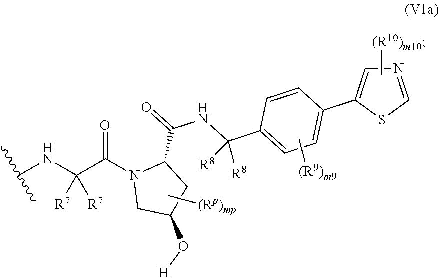
or
VBM is (V2): 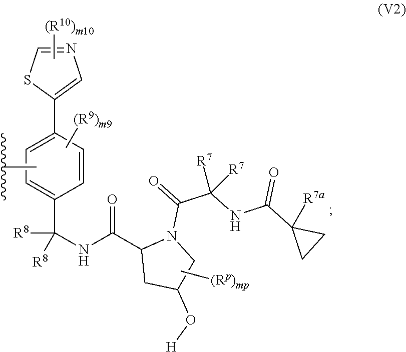
or
VBM is (V2a): 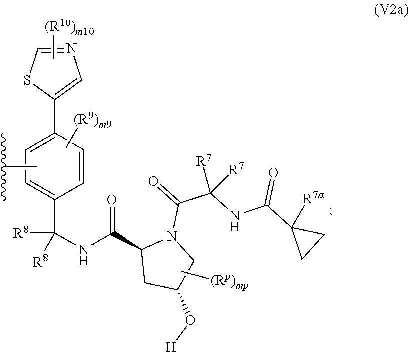
or
VBM is (V3): 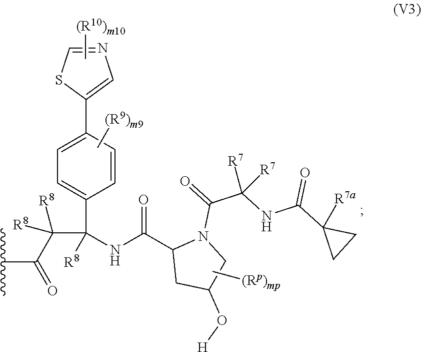
or
VBM is (V3a): 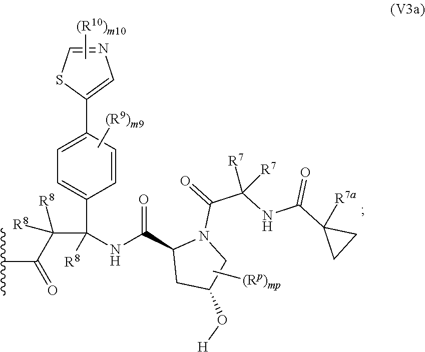
or
VBM is (V4): 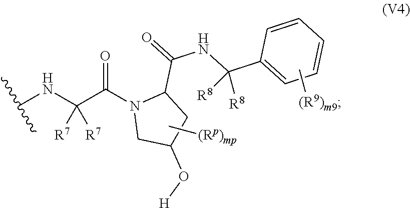
or
VBM is (V4a):

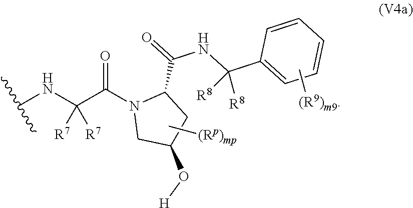

12. The compound of claim 11, wherein one R⁷ is H; and the other R⁷ is C₁₋₆ alkyl; or
one R⁷ is H; and the other R⁷ is tert-butyl.

13. The compound of any one of claim 11 or 12, wherein the carbon atom to which each R⁷ is attached has (S)-stereogenic configuration.

14. The compound of any one of claims 11-13, wherein one R⁸ is H; and the other R⁸ is C₁₋₆ alkyl (e.g., methyl).

15. The compound of any one of claims 11-13, wherein each R⁸ is H.

16. The compound of any one of claims 11-13, wherein the carbon atom to which each R⁸ is attached has (S)-stereogenic configuration.

17. The compound of any one of claims 11-16, wherein m9 is 0.

18. The compound of any one of claims 11-17, wherein m10 is 1.

19. The compound of any one of claims 11-18, wherein R⁰ is C₁₋₆ alkyl; or R¹⁰ is methyl.

20. The compound of any one of claims 11-19, wherein mp is 0.

21. The compound of any one of claims 1-11, wherein VBM is: 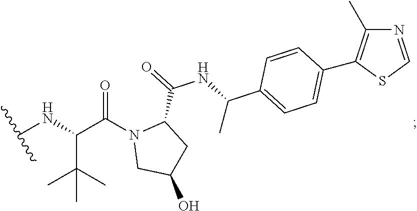 [SMILES: *N[C@H](C(=O)N1C[C@H](O)C[C@H]1C(=O)N[C@@H](C)c1ccc(-c2scnc2C)cc1)C(C)(C)C]
VBM is: 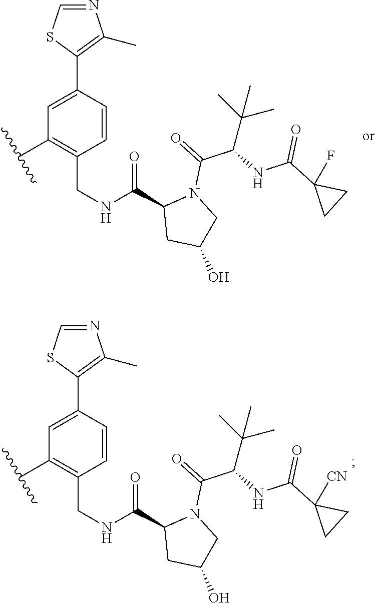 [SMILES: *c1cc(-c2scnc2C)ccc1CNC(=O)[C@@H]1C[C@@H](O)CN1C(=O)[C@@H](NC(=O)C1(F)CC1)C(C)(C)C ; *c1cc(-c2scnc2C)ccc1CNC(=O)[C@@H]1C[C@@H](O)CN1C(=O)[C@@H](NC(=O)C1(C#N)CC1)C(C)(C)C]
VBM is: 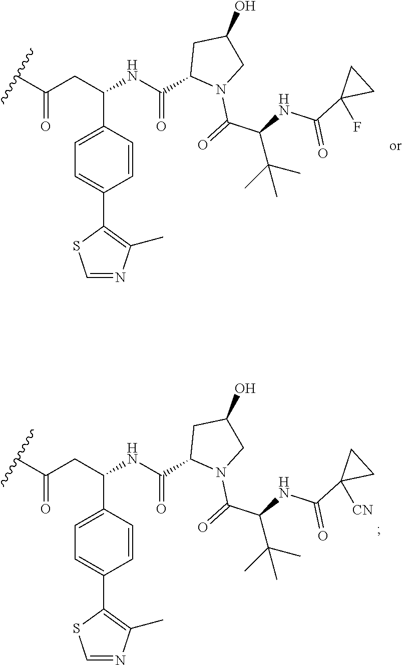 [SMILES: *C(=O)C[C@H](NC(=O)[C@@H]1C[C@@H](O)CN1C(=O)[C@@H](NC(=O)C1(F)CC1)C(C)(C)C)c1ccc(-c2scnc2C)cc1 ; *C(=O)C[C@H](NC(=O)[C@@H]1C[C@@H](O)CN1C(=O)[C@@H](NC(=O)C1(C#N)CC1)C(C)(C)C)c1ccc(-c2scnc2C)cc1]
VBM is:

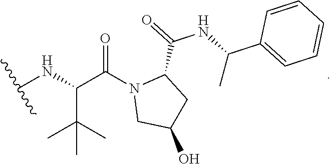 [SMILES: *N[C@H](C(=O)N1C[C@H](O)C[C@H]1C(=O)N[C@@H](C)c1ccccc1)C(C)(C)C]

22. The compound of any one of claims 1-21, wherein L is -(L^{A})ₙ₁-; and L^{A} and n1 are defined according to (BB).

23. The compound of claim 22, wherein n1 is an integer from 3 to 5; or
n1 is an integer from 5 to 9; or n1 is an integer from 9 to 15.

24. The compound of any one of claim 22 or 23, wherein 1-4 occurrences of L^{A} is L^{A3}.

25. The compound of any one of claims 22-24, wherein 1-3 occurrences of L^{A} is L^{A3}.

26. The compound of any one of claims 22-25, wherein 1-2 occurrences of L^{A} is L^{A3}.

27. The compound of any one of claims 22-26, wherein 0-2 occurrences of L^{A1} is —CHR^{L}— or —C(R^{L})₂—; and each remaining occurrence of L^{A1} is —CH₂—.

28. The compound of any one of claims 22-27, wherein each occurrence of L^{A1} is —CH₂—.

29. The compound of any one of claims 22-27, wherein one occurrence of L^{A1} is —CHR^{L}— or —C(R^{L})₂—; and each remaining occurrence of L^{A1} is —CH₂—.

30. The compound of any one of claims 1-29, wherein each R^{L} is independently selected from the group consisting of: —F and —C₁₋₃ alkyl optionally substituted with 1-3 F.

31. The compound of any one of claims 1-22, wherein L is:
-(L^{A3})₀₋₂-(L^{A1})₁₋₁₅-(L^{A3})_{0-1-bb},
wherein bb represents the point of attachment to VBM.

32. The compound of claim 31, wherein 0-2 occurrences of L^{A1} is —CHR^{L}— or —C(R^{L})₂—; and each remaining occurrence of L^{A1} is —CH₂—; or
each occurrence of L^{A1} is —CH₂—; or
one occurrence of L^{A1} is —CHR^{L}— or —C(R^{L})₂—; and each remaining occurrence of L^{A1} is —CH₂—.

33. The compound of any one of claim 31 or 32, wherein each R^{L} is independently selected from the group consisting of: —F and —C₁₋₃ alkyl optionally substituted with 1-3 F.

34. The compound of claim 31, wherein L is a divalent group of Formula (L-1): 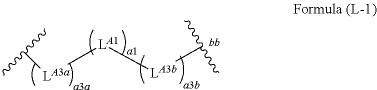
wherein:
a3a is 0 or 1;
L^{A3a} is selected from the group consisting of: —O—, —N(H)—, and —N(C₁₋₃ alkyl)-;
a1 is an integer from 1 to 15;
a3b is 0 or 1;
L^{A3b} is independently selected from the group consisting of: C(═O), —O—, N(H)—, and —N(C₁₋₃ alkyl)-; and
bb represents the point of attachment to VBM.

35. The compound of claim 36, wherein a3b is 1.

36. The compound of claim 36 or 37, wherein L^{A3b} is C(═O).

37. The compound of claim 36 or 37, wherein L^{A3b} is —N(H)—; or
L^{A3b} is —N(C₁₋₃ alkyl)-.

38. The compound of claim 36 or 37, wherein L^{A3b} is —O—.

39. The compound of claim 36, wherein a3b is 0.

40. The compound of any one of claims 36-39, wherein a3a is 1.

41. The compound of any one of claims 36-40, wherein L^{A3a} is 0.

42. The compound of any one of claims 36-41, wherein a3a is 0.

43. The compound of any one of claims 36-42, wherein a1 is 1 or 2; or
a1 is an integer from 3 to 5; or
a1 is an integer from 5 to 9; or
a1 is an integer from 9 to 15.

44. The compound of any one of claims 36-43, wherein 0-2 occurrences of L^{A1} is —CHR^{L}— or —C(R^{L})₂—; and each remaining occurrence of L^{A1} is —CH₂—; or
each occurrence of L^{A1} is —CH₂—; or
one occurrence of L^{A1} is —CHR^{L}— or —C(R^{L})₂—; and each remaining occurrence of L^{A1} is —CH₂—.

45. The compound of claim 44, wherein each R^{L} is independently selected from the group consisting of: —F and —C₁₋₃ alkyl optionally substituted with 1-3 F.

46. The compound of any one of claims 1-21, wherein L is -(L^{A})ₙ₁-; and L^{A} and n1 are defined according to (AA).

47. The compound of claim 46, wherein n1 is an integer from 3 to 5; or
n1 is an integer from 5 to 9; or.
n1 is 6, 7, or 8; or.
n1 is an integer from 9 to 12.

48. The compound of any one of claim 46 or 47, wherein 1-2 occurrences of L^{A} is L^{A4}; or
wherein one occurrence of L^{A} is L^{A4}.

49. The compound of any one of claims 46-48, wherein each L^{A4} is independently selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

50. The compound of any one of claims 46-49, wherein 1-4 occurrences of L^{A} is L^{A3}; or
1-2 occurrences of L^{A} is L^{A3}; or
2-7 occurrences of L^{A} are L^{A1}; or
2-5 occurrences of L^{A} are L^{A1}.

51. The compound of any one of claims 46-50, wherein 0-2 occurrences of L^{A1} is —CHR^{L}— or —C(R^{L})₂—; and each remaining occurrence of L^{A1} is —CH₂—; or
each occurrence of L^{A1} is —CH₂—; or
one occurrence of L^{A1} is —CHR^{L}— or —C(R^{L})₂—; and each remaining occurrence of L^{A1} is —CH₂—.

52. The compound of claim 51, wherein each R^{L} is independently selected from the group consisting of: —F and —C₁₋₃ alkyl optionally substituted with 1-3 F.

53. The compound of any one of claims 1-21 or 46, wherein L is -(L^{A})ₙ₁-, and L^{A} and n1 are defined according to (AA), wherein:
n1 is an integer from 5 to 9;
1-2 occurrences of L^{A} is L^{A4};
2-7 occurrences of L^{A} are L^{A1}; and
1-3 occurrences of L^{A} is L^{A3}.

54. The compound of any one of claims 1-21 or 46, wherein L is selected from the group consisting of:
(i) -(L^{A3})₀₋₂-(L^{A1})₀₋₅-L^{A4}-(L^{A1})₀₋₅-L^{A3}-_{bb};
(ii) -(L^{A3})₀₋₂-(L^{A1})₀₋₅-L^{A4}-(L^{A1})_{0-5-bb}; and
provided that L contains 2-7 L^{A1}; and wherein bb represents the point of attachment to VBM.

55. The compound of claim 53 or 54, wherein each L^{A4} is independently selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

56. The compound of any one of claims 53-55, wherein 0-1 occurrence of L^{A1} is —CHR^{L}— or —C(R^{L})₂—; and each remaining occurrence of L^{A1} is —CH₂—.

57. The compound of any one of claims 1-36 or 71, wherein L is:
-(L^{A3})₀₋₂-(L^{A1})₀₋₅-L^{A4}_A1)₀₋₅-L^{A3}-_{bb};
provided that L contains 2-7 L^{A1}; and wherein bb represents the point of attachment to VBM.

58. The compound of any one of claims 1-21, 46, or 57, wherein L is:
-(L^{A3})₀₋₁-(L^{A1})₀₋₅-L^{A4}_A1)₀₋₅-L^{A3}-_{bb};
provided that L contains 2-5 L^{A1}; and wherein bb represents the point of attachment to VBM.

59. The compound of claim 57 or 58, wherein L is a divalent group of Formula (L-2): 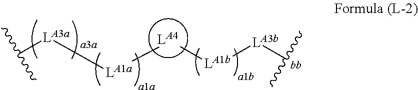
wherein:
a3a is 0 or 1;
L^{A3a} and L^{A3b} are independently selected from the group consisting of: —O—, —N(H)—, —N(C₁₋₃ alkyl)-, and C(═O);
a1a and a1b are independently integers from 0 to 5, provided that a1a+a1b is from 2 to 5;
L^{A1a} and L^{A1}b are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
bb represents the point of attachment to VBM.

60. The compound of claim 59, wherein a3a is 1.

61. The compound of claim 59 or 60, wherein L^{A3a} is —O—.

62. The compound of claim 59, wherein a3a is 0.

63. The compound of any one of claims 59-62, wherein L^{A3b} is —N(H)— or —N(C₁₋₃ alkyl)-; or
L^{A3b} is —O—; or
L^{A3b} is C(═O).

64. The compound of claim 59, wherein a3a is 1; L^{A3a} is —O—; and L^{A3b} is C(═O).

65. The compound of claim 59, wherein a3a is 1; L^{A3a} is —O—; and L^{A3b} is —O—, —N(H)—, or —N(C₁₋₃ alkyl)-.

66. The compound of claim 59, wherein a3a is 0; and L^{A3b} is C(═O).

67. The compound of claim 59, wherein a3a is 0; and L^{A3b} is —O—, —N(H)—, or —N(C₁₋₃ alkyl)-.

68. The compound of any one of claims 1-21 or 46, wherein L is:
-(L^{A3})₀₋₂-(L^{A1})₀₋₅-L^{A4}_A1)_{0-5-bb};
provided that L contains 2-7 L^{A1}; and wherein bb represents the point of attachment to VBM.

69. The compound of any one of claims 1-21, 46, or 68, wherein L is:
-(L^{A3})₀₋₁-(L^{A1})₀₋₅-L^{A4}_A1)_{0-5-bb};
provided that L contains 2-5 L^{A1}; and wherein bb represents the point of attachment to VBM.

70. The compound of claim 68 or 69, wherein L is a divalent group of Formula (L-3): 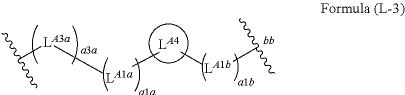
wherein:
a3a is 0 or 1;
L^{A3a} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
bb represents the point of attachment to VBM.

71. The compound of claim 70, wherein a3a is 1.

72. The compound of claim 70 or 71, wherein L^{A3a} is —O—.

73. The compound of claim 70, wherein a3a is 0.

74. The compound of any one of claims 59-67 or 70-73, wherein a1a+a1b is 2, 3, or 4; or
a1a+a1b is 5.

75. The compound of any one of claims 59-67, 70-74, wherein each occurrence of L^{A1a} and L^{A1b} is —CH₂—; or
L^{A1a} is —CHR^{L}— or —C(R^{L})₂—; each remaining occurrence of L^{A1a} is —CH₂—; and each occurrence of L^{A1b} is —CH₂—; or
one occurrence of L^{A1b} is —CHR^{L}— or —C(R^{L})₂—; each remaining occurrence of L^{A1b} is —CH₂—; and each occurrence of L^{A1a} is —CH₂—.

76. The compound of claim 75, wherein each R^{L} is independently selected from the group consisting of: —F and —C₁₋₃ alkyl optionally substituted with 1-3 F.

77. The compound of any one of claims 59-67 or 70-74, wherein a1b is 2, 3, or 4; one occurrence of L^{A1b} is —CHR^{L}— or —C(R^{L})₂—; each remaining occurrence of L^{A1b} is —CH₂—; and each occurrence of L^{A1a} is —CH₂—.

78. The compound of claim 77, wherein one occurrence of L^{A1b} is CF₂, CMe₂, C(H)F, C(H)Me, or C(H)CF₃.

79. The compound of any one of claims 57-78, wherein L^{A4} is 4-10 membered heterocyclylene optionally substituted with 1-3 R^{a}.

80. The compound of any one of claims 57-79, wherein L^{A4} is selected from the group consisting of: 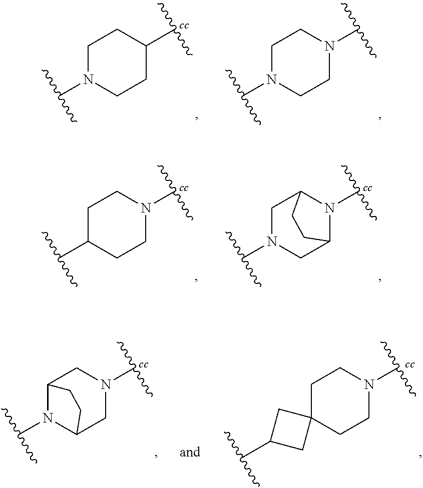 [SMILES: *C1CCN(*)CC1 ; *N1CCN(*)CC1 ; *C1CCN(*)CC1 ; *N1CC2CCC(C1)N2* ; *N1CC2CCC(C1)N2* ; *C1CC2(CCN(*)CC2)C1]
each optionally substituted with 1-3 R^{a} at one or more ring carbon atoms, wherein cc represents the point of attachment to L^{A1b}.

81. The compound of claim 79 or 80, wherein each R^{a} present on L^{A4} is independently F or C₁₋₃ alkyl optionally substituted with 1-3 F.

82. The compound of any one of claims 57-78, wherein L^{A4} is C₃₋₁₀ cycloalkylene optionally substituted with 1-3 R^{a}.

83. The compound of any one of claims 57-78 or 82, wherein L^{A4} is 1,4-cyclohexylene optionally substituted with 1-3 R^{a}.

84. The compound of any one of claims 57-78, wherein L^{A4} is phenylene or 5-6 membered heteroarylene, each of which is optionally substituted with 1-3 R^{a}.

85. The compound of any one of claims 57-78 or 84, wherein L^{A4} is phenylene optionally substituted with 1-3 R^{a}; or
L^{A4} is 1,2-phenylene optionally substituted with 1-3 R^{a}; or
L^{A4} is 1,4-phenylene optionally substituted with 1-3 R^{a}.

86. A compound selected from the group consisting of the compounds in Table C1, or a pharmaceutically acceptable salt thereof.

87. The compound of claim 86, wherein the compound does not exhibit Yₘᵢₙ (6 h)≥70% and/or Yₘᵢₙ (24 h)≥70% under conditions described in Example B1.

88. A pharmaceutical composition comprising a compound as claimed in any one of claims 1-87, or a pharmaceutically acceptable salt thereof, and a pharmaceutically acceptable excipient.

89. A method for treating a cancer in a subject in need thereof, the method comprising administering to the subject a therapeutically effective amount of a compound of any one of claims 1-87, or a pharmaceutically acceptable salt thereof, or a pharmaceutical composition according to claim 88.

90. The method of claim 89, wherein the method comprises administering an additional therapy or therapeutic agent.

91. The method of claim 90, wherein the additional therapy or therapeutic agent is an ALK inhibitor, a BCL-2 inhibitor, a BCR-Abl inhibitor, a BRaf inhibitor, a CDK2 inhibitor, CDK 4/6 inhibitor, a CDK7 inhibitor, a CDK9 inhibitor, an EGFR inhibitor, an anti-EGFR antibody or an anti-EGFR antibody-drug conjugate, an ERK inhibitor, a FGFR1 inhibitor, a FGFR2 inhibitor, a FGFR3 inhibitor, a FGFR4 inhibitor, a HER2 inhibitor, an anti-HER2 antibody or an anti-HER2 antibody-drug conjugate, a JAK inhibitor, a KRas inhibitor, a MEK inhibitor, a MET inhibitor, a PARP inhibitor, an LSD1 inhibitor, a BET inhibitor, a telomerase inhibitor, a TORC1/2 inhibitor, chemotherapy, radiotherapy, or a combination thereof.

92. The method of claim 91, wherein the additional therapy or therapeutic agent is a JAK inhibitor.

93. The method of claim 92, wherein the JAK inhibitor is fedratinib (e.g., fedratinib dihydrochloride monohydrate), momelotinib (e.g., momelotinib dihydrochloride), pacritinib (e.g., pacritinib citrate), ruxolitinib (e.g., ruxolitinib phosphate), or a combination thereof.

94. The method of any one of claims 89-93, wherein the cancer is breast cancer, colorectal cancer, bile duct cancer, colorectal cancer, gastrointestinal stromal tumor, pancreatic cancer, bladder cancer, kidney cancer, cervical cancer, ovarian cancer, uterine cancer, head and neck cancer, hematological cancer, lung cancer, skin cancer, or a combination thereof.

95. The method of claim 94, wherein the hematological cancer is acute lymphocytic leukemia (ALL), acute myeloid leukemia (AML), chronic lymphocytic leukemia (CLL), follicular lymphoma (FL), small lymphocytic lymphoma (SLL), essential thrombocythemia, polycythemia vera, myelofibrosis, or a combination thereof.

96. The method of claim 95, wherein the hematological cancer is essential thrombocythemia, polycythemia vera, myelofibrosis, or a combination thereof.

97. The method of any one of claims 95-96, wherein the hematological cancer has a JAK2 mutation (e.g., JAK2 V617F).

98. Any of the compounds, methods, or uses as provided herein.
